SEQUENCE LISTING (2261 sequences)

SEQ ID NO: 1 - DNA, 22 bp, Artificial Sequence
  primer
gcgggtatggcaatactgaagt

SEQ ID NO: 2 - DNA, 28 bp, Artificial Sequence
  primer
gttttcgtcaaatggaaactcattagga

SEQ ID NO: 3 - DNA, 22 bp, Artificial Sequence
  primer
gcgggtatggcaatactgaagt

SEQ ID NO: 4 - DNA, 26 bp, Artificial Sequence
  primer
tgacacacatttaaacgttggcaaag

SEQ ID NO: 5 - DNA, 22 bp, Artificial Sequence
  primer
gcgggtatggcaatactgaagt

SEQ ID NO: 6 - DNA, 19 bp, Artificial Sequence
  primer
aaggcgacggctttggtat

SEQ ID NO: 7 - DNA, 25 bp, Artificial Sequence
  primer
cacagagctgcaaacaactatacat

SEQ ID NO: 8 - DNA, 28 bp, Artificial Sequence
  primer
gttttcgtcaaatggaaactcattagga

SEQ ID NO: 9 - DNA, 25 bp, Artificial Sequence
  primer
cacagagctgcaaacaactatacat

SEQ ID NO: 10 - DNA, 26 bp, Artificial Sequence
  primer
tgacacacatttaaacgttggcaaag

SEQ ID NO: 11 - DNA, 25 bp, Artificial Sequence
  primer
cacagagctgcaaacaactatacat

SEQ ID NO: 12 - DNA, 19 bp, Artificial Sequence
  primer
aaggcgacggctttggtat

SEQ ID NO: 13 - DNA, 25 bp, Artificial Sequence
  primer
cacagagctgcaaacaactatacat

SEQ ID NO: 14 - DNA, 26 bp, Artificial Sequence
  primer
tgtccagatgtctttgcttttcttca

SEQ ID NO: 15 - DNA, 25 bp, Artificial Sequence
  primer
cacagagctgcaaacaactatacat

SEQ ID NO: 16 - DNA, 24 bp, Artificial Sequence
  primer
tcagttgtctctggttgcaaatct

SEQ ID NO: 17 - DNA, 25 bp, Artificial Sequence
  primer
cacagagctgcaaacaactatacat

SEQ ID NO: 18 - DNA, 26 bp, Artificial Sequence
  primer
ccattaacaggtcttccaaagtacga

SEQ ID NO: 19 - DNA, 23 bp, Artificial Sequence
  primer
gctcacacaaaggacggattaac

SEQ ID NO: 20 - DNA, 26 bp, Artificial Sequence
  primer
atccgtgcttacaaccttagatactg

SEQ ID NO: 21 - DNA, 21 bp, Artificial Sequence
  primer
ggaattgtgtgccccatctgt

SEQ ID NO: 22 - DNA, 28 bp, Artificial Sequence
  primer
gttttcgtcaaatggaaactcattagga

SEQ ID NO: 23 - DNA, 21 bp, Artificial Sequence
  primer
ggaattgtgtgccccatctgt

SEQ ID NO: 24 - DNA, 26 bp, Artificial Sequence
  primer
tgacacacatttaaacgttggcaaag

SEQ ID NO: 25 - DNA, 21 bp, Artificial Sequence
  primer
ggaattgtgtgccccatctgt

SEQ ID NO: 26 - DNA, 19 bp, Artificial Sequence
  primer
aaggcgacggctttggtat

SEQ ID NO: 27 - DNA, 21 bp, Artificial Sequence
  primer
ggaattgtgtgccccatctgt

SEQ ID NO: 28 - DNA, 26 bp, Artificial Sequence
  primer
atccgtgcttacaaccttagatactg

SEQ ID NO: 29 - DNA, 24 bp, Artificial Sequence
  primer
tcagatagtggctatggctgttct

SEQ ID NO: 30 - DNA, 28 bp, Artificial Sequence
  primer
gtcatttatttcatatactggattgcca

SEQ ID NO: 31 - DNA, 24 bp, Artificial Sequence
  primer
tcagatagtggctatggctgttct

SEQ ID NO: 32 - DNA, 21 bp, Artificial Sequence
  primer
ggtttccttcggtgtctgcat

SEQ ID NO: 33 - DNA, 24 bp, Artificial Sequence
  primer
tcagatagtggctatggctgttct

SEQ ID NO: 34 - DNA, 18 bp, Artificial Sequence
  primer
acgtctggccgtaggtct

SEQ ID NO: 35 - DNA, 28 bp, Artificial Sequence
  primer
ttcactgcaagacatagaaataacctgt

SEQ ID NO: 36 - DNA, 28 bp, Artificial Sequence
  primer
gtcatttatttcatatactggattgcca

SEQ ID NO: 37 - DNA, 28 bp, Artificial Sequence
  primer
ttcactgcaagacatagaaataacctgt

SEQ ID NO: 38 - DNA, 21 bp, Artificial Sequence
  primer
ggtttccttcggtgtctgcat

SEQ ID NO: 39 - DNA, 28 bp, Artificial Sequence
  primer
ttcactgcaagacatagaaataacctgt

SEQ ID NO: 40 - DNA, 18 bp, Artificial Sequence
  primer
acgtctggccgtaggtct

SEQ ID NO: 41 - DNA, 28 bp, Artificial Sequence
  primer
ttcactgcaagacatagaaataacctgt

SEQ ID NO: 42 - DNA, 22 bp, Artificial Sequence
  primer
cccagctatgttgtgaaatcgt

SEQ ID NO: 43 - DNA, 28 bp, Artificial Sequence
  primer
ttcactgcaagacatagaaataacctgt

SEQ ID NO: 44 - DNA, 21 bp, Artificial Sequence
  primer
agaaacagctgctggaatgct

SEQ ID NO: 45 - DNA, 22 bp, Artificial Sequence
  primer
ggattggacactgcaagacaca

SEQ ID NO: 46 - DNA, 18 bp, Artificial Sequence
  primer
acgtctggccgtaggtct

SEQ ID NO: 47 - DNA, 22 bp, Artificial Sequence
  primer
cagctacacctacaggcaacaa

SEQ ID NO: 48 - DNA, 27 bp, Artificial Sequence
  primer
gtatttacaactcttgccacagaagga

SEQ ID NO: 49 - DNA, 22 bp, Artificial Sequence
  primer
cagctacacctacaggcaacaa

SEQ ID NO: 50 - DNA, 28 bp, Artificial Sequence
  primer
tcaggtaactgcaccctaaatactctat

SEQ ID NO: 51 - DNA, 27 bp, Artificial Sequence
  primer
cgaaaacatagcgaccactatagagat

SEQ ID NO: 52 - DNA, 27 bp, Artificial Sequence
  primer
gtatttacaactcttgccacagaagga

SEQ ID NO: 53 - DNA, 27 bp, Artificial Sequence
  primer
cgaaaacatagcgaccactatagagat

SEQ ID NO: 54 - DNA, 28 bp, Artificial Sequence
  primer
tcaggtaactgcaccctaaatactctat

SEQ ID NO: 55 - DNA, 20 bp, Artificial Sequence
  primer
tgcatcccagcagtaagcaa

SEQ ID NO: 56 - DNA, 28 bp, Artificial Sequence
  primer
gtcatttatttcatatactggattgcca

SEQ ID NO: 57 - DNA, 20 bp, Artificial Sequence
  primer
tgcatcccagcagtaagcaa

SEQ ID NO: 58 - DNA, 21 bp, Artificial Sequence
  primer
ggtttccttcggtgtctgcat

SEQ ID NO: 59 - DNA, 20 bp, Artificial Sequence
  primer
tgcatcccagcagtaagcaa

SEQ ID NO: 60 - DNA, 18 bp, Artificial Sequence
  primer
acgtctggccgtaggtct

SEQ ID NO: 61 - DNA, 20 bp, Artificial Sequence
  primer
tgcatcccagcagtaagcaa

SEQ ID NO: 62 - DNA, 27 bp, Artificial Sequence
  primer
gtatttacaactcttgccacagaagga

SEQ ID NO: 63 - DNA, 20 bp, Artificial Sequence
  primer
tgcatcccagcagtaagcaa

SEQ ID NO: 64 - DNA, 28 bp, Artificial Sequence
  primer
tcaggtaactgcaccctaaatactctat

SEQ ID NO: 65 - DNA, 22 bp, Artificial Sequence
  primer
gcgggtatggcaatactgaagt

SEQ ID NO: 66 - DNA, 28 bp, Artificial Sequence
  primer
aatgtaaaaaccaccagtctgctatgta

SEQ ID NO: 67 - DNA, 22 bp, Artificial Sequence
  primer
gcgggtatggcaatactgaagt

SEQ ID NO: 68 - DNA, 26 bp, Artificial Sequence
  primer
cgttgagaaagagtctccatcgtttt

SEQ ID NO: 69 - DNA, 22 bp, Artificial Sequence
  primer
gcgggtatggcaatactgaagt

SEQ ID NO: 70 - DNA, 24 bp, Artificial Sequence
  primer
gaattcgatgtggtggtgttgttg

SEQ ID NO: 71 - DNA, 23 bp, Artificial Sequence
  primer
cggcattggaaataccctacgat

SEQ ID NO: 72 - DNA, 28 bp, Artificial Sequence
  primer
aatgtaaaaaccaccagtctgctatgta

SEQ ID NO: 73 - DNA, 23 bp, Artificial Sequence
  primer
cggcattggaaataccctacgat

SEQ ID NO: 74 - DNA, 26 bp, Artificial Sequence
  primer
cgttgagaaagagtctccatcgtttt

SEQ ID NO: 75 - DNA, 23 bp, Artificial Sequence
  primer
cggcattggaaataccctacgat

SEQ ID NO: 76 - DNA, 24 bp, Artificial Sequence
  primer
gaattcgatgtggtggtgttgttg

SEQ ID NO: 77 - DNA, 23 bp, Artificial Sequence
  primer
cggcattggaaataccctacgat

SEQ ID NO: 78 - DNA, 24 bp, Artificial Sequence
  primer
ttttcttctggacacaacggtctt

SEQ ID NO: 79 - DNA, 23 bp, Artificial Sequence
  primer
cggcattggaaataccctacgat

SEQ ID NO: 80 - DNA, 25 bp, Artificial Sequence
  primer
acatagtcttgcaacgtaggtgttt

SEQ ID NO: 81 - DNA, 23 bp, Artificial Sequence
  primer
cggcattggaaataccctacgat

SEQ ID NO: 82 - DNA, 28 bp, Artificial Sequence
  primer
cattaacagctcttgcaatatgcgaata

SEQ ID NO: 83 - DNA, 19 bp, Artificial Sequence
  primer
cagctgcatgcacaaacca

SEQ ID NO: 84 - DNA, 23 bp, Artificial Sequence
  primer
tttagacactgggacaggtggta

SEQ ID NO: 85 - DNA, 19 bp, Artificial Sequence
  primer
aatcgtgtgccccaactgt

SEQ ID NO: 86 - DNA, 28 bp, Artificial Sequence
  primer
aatgtaaaaaccaccagtctgctatgta

SEQ ID NO: 87 - DNA, 19 bp, Artificial Sequence
  primer
aatcgtgtgccccaactgt

SEQ ID NO: 88 - DNA, 26 bp, Artificial Sequence
  primer
cgttgagaaagagtctccatcgtttt

SEQ ID NO: 89 - DNA, 19 bp, Artificial Sequence
  primer
aatcgtgtgccccaactgt

SEQ ID NO: 90 - DNA, 24 bp, Artificial Sequence
  primer
gaattcgatgtggtggtgttgttg

SEQ ID NO: 91 - DNA, 19 bp, Artificial Sequence
  primer
aatcgtgtgccccaactgt

SEQ ID NO: 92 - DNA, 23 bp, Artificial Sequence
  primer
tttagacactgggacaggtggta

SEQ ID NO: 93 - DNA, 25 bp, Artificial Sequence
  primer
gatgagctagaagacagcggatatg

SEQ ID NO: 94 - DNA, 28 bp, Artificial Sequence
  primer
catacactgggttaccattttcatcaaa

SEQ ID NO: 95 - DNA, 25 bp, Artificial Sequence
  primer
gatgagctagaagacagcggatatg

SEQ ID NO: 96 - DNA, 28 bp, Artificial Sequence
  primer
tgatatttcctccatggttttccttgtc

SEQ ID NO: 97 - DNA, 25 bp, Artificial Sequence
  primer
gatgagctagaagacagcggatatg

SEQ ID NO: 98 - DNA, 21 bp, Artificial Sequence
  primer
gtggtggtcggttatcgttgt

SEQ ID NO: 99 - DNA, 28 bp, Artificial Sequence
  primer
agcattggagacaactatacacaacatt

SEQ ID NO: 100 - DNA, 28 bp, Artificial Sequence
  primer
catacactgggttaccattttcatcaaa

SEQ ID NO: 101 - DNA, 28 bp, Artificial Sequence
  primer
agcattggagacaactatacacaacatt

SEQ ID NO: 102 - DNA, 28 bp, Artificial Sequence
  primer
tgatatttcctccatggttttccttgtc

SEQ ID NO: 103 - DNA, 28 bp, Artificial Sequence
  primer
agcattggagacaactatacacaacatt

SEQ ID NO: 104 - DNA, 21 bp, Artificial Sequence
  primer
gtggtggtcggttatcgttgt

SEQ ID NO: 105 - DNA, 28 bp, Artificial Sequence
  primer
agcattggagacaactatacacaacatt

SEQ ID NO: 106 - DNA, 28 bp, Artificial Sequence
  primer
tcgtttgtttaaatccacatgtcgtttt

SEQ ID NO: 107 - DNA, 28 bp, Artificial Sequence
  primer
agcattggagacaactatacacaacatt

SEQ ID NO: 108 - DNA, 26 bp, Artificial Sequence
  primer
catattcctttaacgttggcttgtgt

SEQ ID NO: 109 - DNA, 23 bp, Artificial Sequence
  primer
acgtactgcaactaactgcacaa

SEQ ID NO: 110 - DNA, 27 bp, Artificial Sequence
  primer
atcagtgctgacaactttagatacagg

SEQ ID NO: 111 - DNA, 19 bp, Artificial Sequence
  primer
gtgccctacctgtgcacaa

SEQ ID NO: 112 - DNA, 28 bp, Artificial Sequence
  primer
catacactgggttaccattttcatcaaa

SEQ ID NO: 113 - DNA, 19 bp, Artificial Sequence
  primer
gtgccctacctgtgcacaa

SEQ ID NO: 114 - DNA, 28 bp, Artificial Sequence
  primer
tgatatttcctccatggttttccttgtc

SEQ ID NO: 115 - DNA, 19 bp, Artificial Sequence
  primer
gtgccctacctgtgcacaa

SEQ ID NO: 116 - DNA, 21 bp, Artificial Sequence
  primer
gtggtggtcggttatcgttgt

SEQ ID NO: 117 - DNA, 19 bp, Artificial Sequence
  primer
gtgccctacctgtgcacaa

SEQ ID NO: 118 - DNA, 27 bp, Artificial Sequence
  primer
atcagtgctgacaactttagatacagg

SEQ ID NO: 119 - DNA, 26 bp, Artificial Sequence
  primer
attatttgaactaccagacagcggtt

SEQ ID NO: 120 - DNA, 28 bp, Artificial Sequence
  primer
tcattgtgaaatgtaaagaccactaccc

SEQ ID NO: 121 - DNA, 26 bp, Artificial Sequence
  primer
attatttgaactaccagacagcggtt

SEQ ID NO: 122 - DNA, 26 bp, Artificial Sequence
  primer
ggaaagcgtctccatcattttctttg

SEQ ID NO: 123 - DNA, 26 bp, Artificial Sequence
  primer
attatttgaactaccagacagcggtt

SEQ ID NO: 124 - DNA, 20 bp, Artificial Sequence
  primer
gctttggtatgggtctcggt

SEQ ID NO: 125 - DNA, 27 bp, Artificial Sequence
  primer
cgaggtagaagaaagcatccatgaaat

SEQ ID NO: 126 - DNA, 28 bp, Artificial Sequence
  primer
tcattgtgaaatgtaaagaccactaccc

SEQ ID NO: 127 - DNA, 27 bp, Artificial Sequence
  primer
cgaggtagaagaaagcatccatgaaat

SEQ ID NO: 128 - DNA, 26 bp, Artificial Sequence
  primer
ggaaagcgtctccatcattttctttg

SEQ ID NO: 129 - DNA, 27 bp, Artificial Sequence
  primer
cgaggtagaagaaagcatccatgaaat

SEQ ID NO: 130 - DNA, 20 bp, Artificial Sequence
  primer
gctttggtatgggtctcggt

SEQ ID NO: 131 - DNA, 27 bp, Artificial Sequence
  primer
cgaggtagaagaaagcatccatgaaat

SEQ ID NO: 132 - DNA, 25 bp, Artificial Sequence
  primer
tccaccgatgttatggaatcgtttt

SEQ ID NO: 133 - DNA, 26 bp, Artificial Sequence
  primer
tctacatctgactgcacaaacaaaga

SEQ ID NO: 134 - DNA, 26 bp, Artificial Sequence
  primer
catcagtgctaacaaccttagacact

SEQ ID NO: 135 - DNA, 26 bp, Artificial Sequence
  primer
tctacatctgactgcacaaacaaaga

SEQ ID NO: 136 - DNA, 27 bp, Artificial Sequence
  primer
actctgtattgcaaaccagataccttg

SEQ ID NO: 137 - DNA, 23 bp, Artificial Sequence
  primer
cggctgttcacagagagcataat

SEQ ID NO: 138 - DNA, 28 bp, Artificial Sequence
  primer
tcattgtgaaatgtaaagaccactaccc

SEQ ID NO: 139 - DNA, 23 bp, Artificial Sequence
  primer
cggctgttcacagagagcataat

SEQ ID NO: 140 - DNA, 26 bp, Artificial Sequence
  primer
ggaaagcgtctccatcattttctttg

SEQ ID NO: 141 - DNA, 23 bp, Artificial Sequence
  primer
cggctgttcacagagagcataat

SEQ ID NO: 142 - DNA, 20 bp, Artificial Sequence
  primer
gctttggtatgggtctcggt

SEQ ID NO: 143 - DNA, 23 bp, Artificial Sequence
  primer
cggctgttcacagagagcataat

SEQ ID NO: 144 - DNA, 26 bp, Artificial Sequence
  primer
catcagtgctaacaaccttagacact

SEQ ID NO: 145 - DNA, 23 bp, Artificial Sequence
  primer
cggctgttcacagagagcataat

SEQ ID NO: 146 - DNA, 27 bp, Artificial Sequence
  primer
actctgtattgcaaaccagataccttg

SEQ ID NO: 147 - DNA, 20 bp, Artificial Sequence
  primer
ggtgtattccgtgccagaca

SEQ ID NO: 148 - DNA, 28 bp, Artificial Sequence
  primer
ctgttttggtcaaatggaaatgcattag

SEQ ID NO: 149 - DNA, 20 bp, Artificial Sequence
  primer
ggtgtattccgtgccagaca

SEQ ID NO: 150 - DNA, 21 bp, Artificial Sequence
  primer
ggtcgcggtggtgtttgataa

SEQ ID NO: 151 - DNA, 25 bp, Artificial Sequence
  primer
caccaccttgcaggacattacaata

SEQ ID NO: 152 - DNA, 28 bp, Artificial Sequence
  primer
ctgttttggtcaaatggaaatgcattag

SEQ ID NO: 153 - DNA, 25 bp, Artificial Sequence
  primer
caccaccttgcaggacattacaata

SEQ ID NO: 154 - DNA, 21 bp, Artificial Sequence
  primer
ggtcgcggtggtgtttgataa

SEQ ID NO: 155 - DNA, 25 bp, Artificial Sequence
  primer
caccaccttgcaggacattacaata

SEQ ID NO: 156 - DNA, 27 bp, Artificial Sequence
  primer
ctgtcctgtatagcttcctgctatttt

SEQ ID NO: 157 - DNA, 25 bp, Artificial Sequence
  primer
caccaccttgcaggacattacaata

SEQ ID NO: 158 - DNA, 23 bp, Artificial Sequence
  primer
tgctgtagttgtcgcagagtatc

SEQ ID NO: 159 - DNA, 22 bp, Artificial Sequence
  primer
cacagtaacagtacaggccaca

SEQ ID NO: 160 - DNA, 22 bp, Artificial Sequence
  primer
agtattgacaaccttcgccaca

SEQ ID NO: 161 - DNA, 18 bp, Artificial Sequence
  primer
cgtggtgtgcaactgcaa

SEQ ID NO: 162 - DNA, 28 bp, Artificial Sequence
  primer
ctgttttggtcaaatggaaatgcattag

SEQ ID NO: 163 - DNA, 18 bp, Artificial Sequence
  primer
cgtggtgtgcaactgcaa

SEQ ID NO: 164 - DNA, 21 bp, Artificial Sequence
  primer
ggtcgcggtggtgtttgataa

SEQ ID NO: 165 - DNA, 18 bp, Artificial Sequence
  primer
cgtggtgtgcaactgcaa

SEQ ID NO: 166 - DNA, 22 bp, Artificial Sequence
  primer
agtattgacaaccttcgccaca

SEQ ID NO: 167 - DNA, 24 bp, Artificial Sequence
  primer
tcagatagtggctatggctgttct

SEQ ID NO: 168 - DNA, 28 bp, Artificial Sequence
  primer
gaaatgcatgtggaaatgtaaataccgt

SEQ ID NO: 169 - DNA, 24 bp, Artificial Sequence
  primer
tcagatagtggctatggctgttct

SEQ ID NO: 170 - DNA, 21 bp, Artificial Sequence
  primer
ggattccttcggtgtctgcat

SEQ ID NO: 171 - DNA, 24 bp, Artificial Sequence
  primer
tcagatagtggctatggctgttct

SEQ ID NO: 172 - DNA, 18 bp, Artificial Sequence
  primer
cccacggatgcggttttg

SEQ ID NO: 173 - DNA, 26 bp, Artificial Sequence
  primer
ctacaagacgtatctattgcctgtgt

SEQ ID NO: 174 - DNA, 28 bp, Artificial Sequence
  primer
gaaatgcatgtggaaatgtaaataccgt

SEQ ID NO: 175 - DNA, 26 bp, Artificial Sequence
  primer
ctacaagacgtatctattgcctgtgt

SEQ ID NO: 176 - DNA, 21 bp, Artificial Sequence
  primer
ggattccttcggtgtctgcat

SEQ ID NO: 177 - DNA, 26 bp, Artificial Sequence
  primer
ctacaagacgtatctattgcctgtgt

SEQ ID NO: 178 - DNA, 18 bp, Artificial Sequence
  primer
cccacggatgcggttttg

SEQ ID NO: 179 - DNA, 26 bp, Artificial Sequence
  primer
ctacaagacgtatctattgcctgtgt

SEQ ID NO: 180 - DNA, 27 bp, Artificial Sequence
  primer
cgtttgtccttaaggtgtctacgtttt

SEQ ID NO: 181 - DNA, 26 bp, Artificial Sequence
  primer
ctacaagacgtatctattgcctgtgt

SEQ ID NO: 182 - DNA, 25 bp, Artificial Sequence
  primer
tcaaaaacagctgctgtagtgttct

SEQ ID NO: 183 - DNA, 28 bp, Artificial Sequence
  primer
tcctgtgttcaagtacaagtaacaacaa

SEQ ID NO: 184 - DNA, 19 bp, Artificial Sequence
  primer
gctgacaactctggccaca

SEQ ID NO: 185 - DNA, 27 bp, Artificial Sequence
  primer
cgcaaatatgcagaccattactcagaa

SEQ ID NO: 186 - DNA, 19 bp, Artificial Sequence
  primer
gctgacaactctggccaca

SEQ ID NO: 187 - DNA, 21 bp, Artificial Sequence
  primer
agcaccttgtcctttgtgtgt

SEQ ID NO: 188 - DNA, 28 bp, Artificial Sequence
  primer
gaaatgcatgtggaaatgtaaataccgt

SEQ ID NO: 189 - DNA, 21 bp, Artificial Sequence
  primer
agcaccttgtcctttgtgtgt

SEQ ID NO: 190 - DNA, 21 bp, Artificial Sequence
  primer
ggattccttcggtgtctgcat

SEQ ID NO: 191 - DNA, 21 bp, Artificial Sequence
  primer
agcaccttgtcctttgtgtgt

SEQ ID NO: 192 - DNA, 18 bp, Artificial Sequence
  primer
cccacggatgcggttttg

SEQ ID NO: 193 - DNA, 21 bp, Artificial Sequence
  primer
agcaccttgtcctttgtgtgt

SEQ ID NO: 194 - DNA, 19 bp, Artificial Sequence
  primer
gctgacaactctggccaca

SEQ ID NO: 195 - DNA, 21 bp, Artificial Sequence
  primer
cggacagcggatatggcaata

SEQ ID NO: 196 - DNA, 28 bp, Artificial Sequence
  primer
tcattcaatgtatacacagcattcccat

SEQ ID NO: 197 - DNA, 21 bp, Artificial Sequence
  primer
cggacagcggatatggcaata

SEQ ID NO: 198 - DNA, 18 bp, Artificial Sequence
  primer
ccacgcaggtggtaaggg

SEQ ID NO: 199 - DNA, 27 bp, Artificial Sequence
  primer
ctgcatgaattatgtgaagctttgaac

SEQ ID NO: 200 - DNA, 28 bp, Artificial Sequence
  primer
tcattcaatgtatacacagcattcccat

SEQ ID NO: 201 - DNA, 27 bp, Artificial Sequence
  primer
ctgcatgaattatgtgaagctttgaac

SEQ ID NO: 202 - DNA, 18 bp, Artificial Sequence
  primer
ccacgcaggtggtaaggg

SEQ ID NO: 203 - DNA, 27 bp, Artificial Sequence
  primer
ctgcatgaattatgtgaagctttgaac

SEQ ID NO: 204 - DNA, 24 bp, Artificial Sequence
  primer
tcccgctatttcatggaacctttt

SEQ ID NO: 205 - DNA, 27 bp, Artificial Sequence
  primer
ctgcatgaattatgtgaagctttgaac

SEQ ID NO: 206 - DNA, 21 bp, Artificial Sequence
  primer
catctgctgtacaacgcgaag

SEQ ID NO: 207 - DNA, 21 bp, Artificial Sequence
  primer
ctaacactggagggcaccaaa

SEQ ID NO: 208 - DNA, 21 bp, Artificial Sequence
  primer
caattcgagacacaggtgcag

SEQ ID NO: 209 - DNA, 20 bp, Artificial Sequence
  primer
gggcgaactaagcctggttt

SEQ ID NO: 210 - DNA, 28 bp, Artificial Sequence
  primer
tcattcaatgtatacacagcattcccat

SEQ ID NO: 211 - DNA, 20 bp, Artificial Sequence
  primer
gggcgaactaagcctggttt

SEQ ID NO: 212 - DNA, 18 bp, Artificial Sequence
  primer
ccacgcaggtggtaaggg

SEQ ID NO: 213 - DNA, 20 bp, Artificial Sequence
  primer
gggcgaactaagcctggttt

SEQ ID NO: 214 - DNA, 21 bp, Artificial Sequence
  primer
caattcgagacacaggtgcag

SEQ ID NO: 215 - DNA, 24 bp, Artificial Sequence
  primer
caaaccatgtcacgtagaagacag

SEQ ID NO: 216 - DNA, 28 bp, Artificial Sequence
  primer
gggtttttgaaatgaaacacaaccaatc

SEQ ID NO: 217 - DNA, 24 bp, Artificial Sequence
  primer
caaaccatgtcacgtagaagacag

SEQ ID NO: 218 - DNA, 23 bp, Artificial Sequence
  primer
cggtatcgactccatcgttttcc

SEQ ID NO: 219 - DNA, 24 bp, Artificial Sequence
  primer
caaaccatgtcacgtagaagacag

SEQ ID NO: 220 - DNA, 19 bp, Artificial Sequence
  primer
gcggaggtcttggaggttt

SEQ ID NO: 221 - DNA, 24 bp, Artificial Sequence
  primer
agaatcggtgcatgaaataaggct

SEQ ID NO: 222 - DNA, 28 bp, Artificial Sequence
  primer
gggtttttgaaatgaaacacaaccaatc

SEQ ID NO: 223 - DNA, 24 bp, Artificial Sequence
  primer
agaatcggtgcatgaaataaggct

SEQ ID NO: 224 - DNA, 23 bp, Artificial Sequence
  primer
cggtatcgactccatcgttttcc

SEQ ID NO: 225 - DNA, 24 bp, Artificial Sequence
  primer
agaatcggtgcatgaaataaggct

SEQ ID NO: 226 - DNA, 19 bp, Artificial Sequence
  primer
gcggaggtcttggaggttt

SEQ ID NO: 227 - DNA, 24 bp, Artificial Sequence
  primer
agaatcggtgcatgaaataaggct

SEQ ID NO: 228 - DNA, 27 bp, Artificial Sequence
  primer
cgcttgtttgcattaacatgtctttct

SEQ ID NO: 229 - DNA, 24 bp, Artificial Sequence
  primer
agaatcggtgcatgaaataaggct

SEQ ID NO: 230 - DNA, 28 bp, Artificial Sequence
  primer
tcagttgtttcaggttgcagatctaata

SEQ ID NO: 231 - DNA, 24 bp, Artificial Sequence
  primer
agaatcggtgcatgaaataaggct

SEQ ID NO: 232 - DNA, 24 bp, Artificial Sequence
  primer
gcatttgctgtagagtacgaaggt

SEQ ID NO: 233 - DNA, 21 bp, Artificial Sequence
  primer
tcactgcaactgagtgcacaa

SEQ ID NO: 234 - DNA, 26 bp, Artificial Sequence
  primer
tgcttacaaccttagagacaggtaca

SEQ ID NO: 235 - DNA, 21 bp, Artificial Sequence
  primer
tcactgcaactgagtgcacaa

SEQ ID NO: 236 - DNA, 20 bp, Artificial Sequence
  primer
cctgtattgcaggccagaca

SEQ ID NO: 237 - DNA, 22 bp, Artificial Sequence
  primer
gctgttgggcacattacaagtt

SEQ ID NO: 238 - DNA, 28 bp, Artificial Sequence
  primer
gggtttttgaaatgaaacacaaccaatc

SEQ ID NO: 239 - DNA, 22 bp, Artificial Sequence
  primer
gctgttgggcacattacaagtt

SEQ ID NO: 240 - DNA, 23 bp, Artificial Sequence
  primer
cggtatcgactccatcgttttcc

SEQ ID NO: 241 - DNA, 22 bp, Artificial Sequence
  primer
gctgttgggcacattacaagtt

SEQ ID NO: 242 - DNA, 19 bp, Artificial Sequence
  primer
gcggaggtcttggaggttt

SEQ ID NO: 243 - DNA, 22 bp, Artificial Sequence
  primer
gctgttgggcacattacaagtt

SEQ ID NO: 244 - DNA, 26 bp, Artificial Sequence
  primer
tgcttacaaccttagagacaggtaca

SEQ ID NO: 245 - DNA, 22 bp, Artificial Sequence
  primer
gctgttgggcacattacaagtt

SEQ ID NO: 246 - DNA, 20 bp, Artificial Sequence
  primer
cctgtattgcaggccagaca

SEQ ID NO: 247 - DNA, 22 bp, Artificial Sequence
  primer
caagacagcgggtatggcaata

SEQ ID NO: 248 - DNA, 30 bp, Artificial Sequence
  primer
tgaaactgaaacactaacattctactgtgt

SEQ ID NO: 249 - DNA, 22 bp, Artificial Sequence
  primer
caagacagcgggtatggcaata

SEQ ID NO: 250 - DNA, 26 bp, Artificial Sequence
  primer
ttttctttgtcctcgtcgttatccaa

SEQ ID NO: 251 - DNA, 22 bp, Artificial Sequence
  primer
caagacagcgggtatggcaata

SEQ ID NO: 252 - DNA, 18 bp, Artificial Sequence
  primer
ggtggtggtggtggtctt

SEQ ID NO: 253 - DNA, 25 bp, Artificial Sequence
  primer
gcaccacttgagtgaggtattagaa

SEQ ID NO: 254 - DNA, 30 bp, Artificial Sequence; identical to SEQ ID NO: 248
  primer

SEQ ID NO: 255 - DNA, 25 bp, Artificial Sequence
  primer
gcaccacttgagtgaggtattagaa

SEQ ID NO: 256 - DNA, 26 bp, Artificial Sequence
  primer
ttttctttgtcctcgtcgttatccaa

SEQ ID NO: 257 - DNA, 25 bp, Artificial Sequence
  primer
gcaccacttgagtgaggtattagaa

SEQ ID NO: 258 - DNA, 18 bp, Artificial Sequence
  primer
ggtggtggtggtggtctt

SEQ ID NO: 259 - DNA, 25 bp, Artificial Sequence
  primer
gcaccacttgagtgaggtattagaa

SEQ ID NO: 260 - DNA, 24 bp, Artificial Sequence
  primer
caattgcttttcctccggagttaa

SEQ ID NO: 261 - DNA, 25 bp, Artificial Sequence
  primer
gcaccacttgagtgaggtattagaa

SEQ ID NO: 262 - DNA, 19 bp, Artificial Sequence
  primer
acgtcttgcagcgttggta

SEQ ID NO: 263 - DNA, 25 bp, Artificial Sequence
  primer
gcaccacttgagtgaggtattagaa

SEQ ID NO: 264 - DNA, 20 bp, Artificial Sequence
  primer
tgtacaacacgcaggtcctc

SEQ ID NO: 265 - DNA, 23 bp, Artificial Sequence
  primer
acaacaaccaccctggtgataag

SEQ ID NO: 266 - DNA, 24 bp, Artificial Sequence
  primer
acaacctttgaaacaggtgttgga

SEQ ID NO: 267 - DNA, 23 bp, Artificial Sequence
  primer
acaacaaccaccctggtgataag

SEQ ID NO: 268 - DNA, 28 bp, Artificial Sequence
  primer
caaccgtaccctaaataccctatattga

SEQ ID NO: 269 - DNA, 23 bp, Artificial Sequence
  primer
gttaacagtaacgtgcccactct

SEQ ID NO: 270 - DNA, 30 bp, Artificial Sequence; identical to SEQ ID NO: 248
  primer

SEQ ID NO: 271 - DNA, 23 bp, Artificial Sequence
  primer
gttaacagtaacgtgcccactct

SEQ ID NO: 272 - DNA, 26 bp, Artificial Sequence
  primer
ttttctttgtcctcgtcgttatccaa

SEQ ID NO: 273 - DNA, 23 bp, Artificial Sequence
  primer
gttaacagtaacgtgcccactct

SEQ ID NO: 274 - DNA, 18 bp, Artificial Sequence
  primer
ggtggtggtggtggtctt

SEQ ID NO: 275 - DNA, 23 bp, Artificial Sequence
  primer
gttaacagtaacgtgcccactct

SEQ ID NO: 276 - DNA, 24 bp, Artificial Sequence
  primer
acaacctttgaaacaggtgttgga

SEQ ID NO: 277 - DNA, 23 bp, Artificial Sequence
  primer
gttaacagtaacgtgcccactct

SEQ ID NO: 278 - DNA, 28 bp, Artificial Sequence
  primer
caaccgtaccctaaataccctatattga

SEQ ID NO: 279 - DNA, 28 bp, Artificial Sequence
  primer
aaaattattgagctagaagacagcggat

SEQ ID NO: 280 - DNA, 31 bp, Artificial Sequence
  primer
tgcatcaaatggaaatggattgttaaattca

SEQ ID NO: 281 - DNA, 28 bp, Artificial Sequence
  primer
aaaattattgagctagaagacagcggat

SEQ ID NO: 282 - DNA, 28 bp, Artificial Sequence
  primer
tgatatttcctccatcgttttccttgtc

SEQ ID NO: 283 - DNA, 28 bp, Artificial Sequence
  primer
aaaattattgagctagaagacagcggat

SEQ ID NO: 284 - DNA, 23 bp, Artificial Sequence
  primer
ccctgtgtactttcgttgttggt

SEQ ID NO: 285 - DNA, 20 bp, Artificial Sequence
  primer
gtcaggcgttggagacatct

SEQ ID NO: 286 - DNA, 31 bp, Artificial Sequence; identical to SEQ ID NO: 280
  primer

SEQ ID NO: 287 - DNA, 20 bp, Artificial Sequence
  primer
gtcaggcgttggagacatct

SEQ ID NO: 288 - DNA, 28 bp, Artificial Sequence
  primer
tgatatttcctccatcgttttccttgtc

SEQ ID NO: 289 - DNA, 20 bp, Artificial Sequence
  primer
gtcaggcgttggagacatct

SEQ ID NO: 290 - DNA, 23 bp, Artificial Sequence
  primer
ccctgtgtactttcgttgttggt

SEQ ID NO: 291 - DNA, 20 bp, Artificial Sequence
  primer
gtcaggcgttggagacatct

SEQ ID NO: 292 - DNA, 28 bp, Artificial Sequence
  primer
cgacccgaaatattatgaaaccttttgt

SEQ ID NO: 293 - DNA, 20 bp, Artificial Sequence
  primer
gtcaggcgttggagacatct

SEQ ID NO: 294 - DNA, 21 bp, Artificial Sequence
  primer
gcgttgggttgtttcctctca

SEQ ID NO: 295 - DNA, 27 bp, Artificial Sequence
  primer
gaggaggactacacagtacaactaact

SEQ ID NO: 296 - DNA, 23 bp, Artificial Sequence
  primer
gcttacaaccttagacacaggca

SEQ ID NO: 297 - DNA, 23 bp, Artificial Sequence
  primer
tgcttatgggcacatgtaccatt

SEQ ID NO: 298 - DNA, 31 bp, Artificial Sequence; identical to SEQ ID NO: 280
  primer

SEQ ID NO: 299 - DNA, 23 bp, Artificial Sequence
  primer
tgcttatgggcacatgtaccatt

SEQ ID NO: 300 - DNA, 28 bp, Artificial Sequence
  primer
tgatatttcctccatcgttttccttgtc

SEQ ID NO: 301 - DNA, 23 bp, Artificial Sequence
  primer
tgcttatgggcacatgtaccatt

SEQ ID NO: 302 - DNA, 23 bp, Artificial Sequence
  primer
ccctgtgtactttcgttgttggt

SEQ ID NO: 303 - DNA, 23 bp, Artificial Sequence
  primer
tgcttatgggcacatgtaccatt

SEQ ID NO: 304 - DNA, 23 bp, Artificial Sequence
  primer
gcttacaaccttagacacaggca

SEQ ID NO: 305 - DNA, 27 bp, Artificial Sequence
  primer
aaagaaggttaataacagtgccagaca

SEQ ID NO: 306 - DNA, 31 bp, Artificial Sequence
  primer
tctatttttgtcaaatggcaatttgtttgga

SEQ ID NO: 307 - DNA, 27 bp, Artificial Sequence
  primer
aaagaaggttaataacagtgccagaca

SEQ ID NO: 308 - DNA, 21 bp, Artificial Sequence
  primer
ggtgtccatcactgtctgcat

SEQ ID NO: 309 - DNA, 27 bp, Artificial Sequence
  primer
aaagaaggttaataacagtgccagaca

SEQ ID NO: 310 - DNA, 19 bp, Artificial Sequence
  primer
cccaagtacgtggcttcgg

SEQ ID NO: 311 - DNA, 25 bp, Artificial Sequence
  primer
gcatcaattgtgtgttttgcaaagg

SEQ ID NO: 312 - DNA, 31 bp, Artificial Sequence; identical to SEQ ID NO: 306
  primer

SEQ ID NO: 313 - DNA, 25 bp, Artificial Sequence
  primer
gcatcaattgtgtgttttgcaaagg

SEQ ID NO: 314 - DNA, 21 bp, Artificial Sequence
  primer
ggtgtccatcactgtctgcat

SEQ ID NO: 315 - DNA, 25 bp, Artificial Sequence
  primer
gcatcaattgtgtgttttgcaaagg

SEQ ID NO: 316 - DNA, 19 bp, Artificial Sequence
  primer
cccaagtacgtggcttcgg

SEQ ID NO: 317 - DNA, 25 bp, Artificial Sequence
  primer
gcatcaattgtgtgttttgcaaagg

SEQ ID NO: 318 - DNA, 19 bp, Artificial Sequence
  primer
tgtaaggctcgcaatccgt

SEQ ID NO: 319 - DNA, 19 bp, Artificial Sequence
  primer
tccgtttgcatccaggcaa

SEQ ID NO: 320 - DNA, 25 bp, Artificial Sequence
  primer
tgacatactcatcagtgctgacaac

SEQ ID NO: 321 - DNA, 19 bp, Artificial Sequence
  primer
tccgtttgcatccaggcaa

SEQ ID NO: 322 - DNA, 27 bp, Artificial Sequence
  primer
gccaaatttattgggatcaggtaactt

SEQ ID NO: 323 - DNA, 25 bp, Artificial Sequence
  primer
actatcctttgtgtgtcctttgtgt

SEQ ID NO: 324 - DNA, 31 bp, Artificial Sequence; identical to SEQ ID NO: 306
  primer

SEQ ID NO: 325 - DNA, 25 bp, Artificial Sequence
  primer
actatcctttgtgtgtcctttgtgt

SEQ ID NO: 326 - DNA, 21 bp, Artificial Sequence
  primer
ggtgtccatcactgtctgcat

SEQ ID NO: 327 - DNA, 25 bp, Artificial Sequence
  primer
actatcctttgtgtgtcctttgtgt

SEQ ID NO: 328 - DNA, 19 bp, Artificial Sequence
  primer
cccaagtacgtggcttcgg

SEQ ID NO: 329 - DNA, 25 bp, Artificial Sequence
  primer
actatcctttgtgtgtcctttgtgt

SEQ ID NO: 330 - DNA, 25 bp, Artificial Sequence
  primer
tgacatactcatcagtgctgacaac

SEQ ID NO: 331 - DNA, 25 bp, Artificial Sequence
  primer
actatcctttgtgtgtcctttgtgt

SEQ ID NO: 332 - DNA, 27 bp, Artificial Sequence
  primer
gccaaatttattgggatcaggtaactt

SEQ ID NO: 333 - DNA, 22 bp, Artificial Sequence
  primer
gaagacagcgggtatggcaata

SEQ ID NO: 334 - DNA, 31 bp, Artificial Sequence
  primer
cattacttaattcatacacaggattaccatt

SEQ ID NO: 335 - DNA, 22 bp, Artificial Sequence
  primer
gaagacagcgggtatggcaata

SEQ ID NO: 336 - DNA, 26 bp, Artificial Sequence
  primer
ttttctttgtcctcgtcgttatccaa

SEQ ID NO: 337 - DNA, 26 bp, Artificial Sequence
  primer
gacagggagacagctcaacaattatt

SEQ ID NO: 338 - DNA, 25 bp, Artificial Sequence
  primer
ctctcggtacacagtttgctgatta

SEQ ID NO: 339 - DNA, 22 bp, Artificial Sequence
  primer
gaagacagcgggtatggcaata

SEQ ID NO: 340 - DNA, 18 bp, Artificial Sequence
  primer
ggtggtggtggtcctgtg

SEQ ID NO: 341 - DNA, 23 bp, Artificial Sequence
  primer
caccatctgagcgaggtattaca

SEQ ID NO: 342 - DNA, 31 bp, Artificial Sequence; identical to SEQ ID NO: 334
  primer

SEQ ID NO: 343 - DNA, 23 bp, Artificial Sequence
  primer
caccatctgagcgaggtattaca

SEQ ID NO: 344 - DNA, 26 bp, Artificial Sequence
  primer
ttttctttgtcctcgtcgttatccaa

SEQ ID NO: 345 - DNA, 23 bp, Artificial Sequence
  primer
caccatctgagcgaggtattaca

SEQ ID NO: 346 - DNA, 18 bp, Artificial Sequence
  primer
ggtggtggtggtcctgtg

SEQ ID NO: 347 - DNA, 23 bp, Artificial Sequence
  primer
caccatctgagcgaggtattaca

SEQ ID NO: 348 - DNA, 28 bp, Artificial Sequence
  primer
gaaatcgtcttttatgttcacagtgcaa

SEQ ID NO: 349 - DNA, 23 bp, Artificial Sequence
  primer
caccatctgagcgaggtattaca

SEQ ID NO: 350 - DNA, 22 bp, Artificial Sequence
  primer
aacctcttgcaacgttggtact

SEQ ID NO: 351 - DNA, 23 bp, Artificial Sequence
  primer
caccatctgagcgaggtattaca

SEQ ID NO: 352 - DNA, 21 bp, Artificial Sequence
  primer
tgtaccacacgtagctcctct

SEQ ID NO: 353 - DNA, 23 bp, Artificial Sequence
  primer
gtatcaacacacaaagccactgt

SEQ ID NO: 354 - DNA, 24 bp, Artificial Sequence
  primer
acaacctttgaaacaggtgttgga

SEQ ID NO: 355 - DNA, 23 bp, Artificial Sequence
  primer
gttaacagtaacgtgcccactct

SEQ ID NO: 356 - DNA, 31 bp, Artificial Sequence; identical to SEQ ID NO: 334
  primer

SEQ ID NO: 357 - DNA, 23 bp, Artificial Sequence
  primer
gttaacagtaacgtgcccactct

SEQ ID NO: 358 - DNA, 26 bp, Artificial Sequence
  primer
ttttctttgtcctcgtcgttatccaa

SEQ ID NO: 359 - DNA, 23 bp, Artificial Sequence
  primer
gttaacagtaacgtgcccactct

SEQ ID NO: 360 - DNA, 18 bp, Artificial Sequence
  primer
ggtggtggtggtcctgtg

SEQ ID NO: 361 - DNA, 23 bp, Artificial Sequence
  primer
gttaacagtaacgtgcccactct

SEQ ID NO: 362 - DNA, 24 bp, Artificial Sequence
  primer
acaacctttgaaacaggtgttgga

SEQ ID NO: 363 - DNA, 21 bp, Artificial Sequence
  primer
agacaaccggcgtatacagtg

SEQ ID NO: 364 - DNA, 28 bp, Artificial Sequence
  primer
ctgttttggtcaaatggaaatgcattag

SEQ ID NO: 365 - DNA, 21 bp, Artificial Sequence
  primer
agacaaccggcgtatacagtg

SEQ ID NO: 366 - DNA, 19 bp, Artificial Sequence
  primer
tcgcggtggtgttctgtag

SEQ ID NO: 367 - DNA, 25 bp, Artificial Sequence
  primer
gacattggacactacattgcatgac

SEQ ID NO: 368 - DNA, 28 bp, Artificial Sequence
  primer
ctgttttggtcaaatggaaatgcattag

SEQ ID NO: 369 - DNA, 25 bp, Artificial Sequence
  primer
gacattggacactacattgcatgac

SEQ ID NO: 370 - DNA, 26 bp, Artificial Sequence
  primer
cttcgttttgttgttaggtgccttag

SEQ ID NO: 371 - DNA, 25 bp, Artificial Sequence
  primer
gacattggacactacattgcatgac

SEQ ID NO: 372 - DNA, 19 bp, Artificial Sequence
  primer
tcgcggtggtgttctgtag

SEQ ID NO: 373 - DNA, 25 bp, Artificial Sequence
  primer
gacattggacactacattgcatgac

SEQ ID NO: 374 - DNA, 21 bp, Artificial Sequence
  primer
ctgttgtagtgtccgcaggtt

SEQ ID NO: 375 - DNA, 23 bp, Artificial Sequence
  primer
agtagaagtgcaggccaaaacaa

SEQ ID NO: 376 - DNA, 21 bp, Artificial Sequence
  primer
attgacaaccttcgccactga

SEQ ID NO: 377 - DNA, 19 bp, Artificial Sequence
  primer
tccgtggtgtgcaactgaa

SEQ ID NO: 378 - DNA, 28 bp, Artificial Sequence
  primer
ctgttttggtcaaatggaaatgcattag

SEQ ID NO: 379 - DNA, 19 bp, Artificial Sequence
  primer
tccgtggtgtgcaactgaa

SEQ ID NO: 380 - DNA, 19 bp, Artificial Sequence
  primer
tcgcggtggtgttctgtag

SEQ ID NO: 381 - DNA, 19 bp, Artificial Sequence
  primer
tccgtggtgtgcaactgaa

SEQ ID NO: 382 - DNA, 21 bp, Artificial Sequence
  primer
attgacaaccttcgccactga

SEQ ID NO: 383 - DNA, 23 bp, Artificial Sequence
  primer
aaacgaagactgtttgaggagca

SEQ ID NO: 384 - DNA, 24 bp, Artificial Sequence
  primer
gggttcccattactgtcaaatgga

SEQ ID NO: 385 - DNA, 23 bp, Artificial Sequence
  primer
aaacgaagactgtttgaggagca

SEQ ID NO: 386 - DNA, 19 bp, Artificial Sequence
  primer
tggtgttggtggttgtggt

SEQ ID NO: 387 - DNA, 28 bp, Artificial Sequence
  primer
agcgttatgtgacgaagtgaatatttct

SEQ ID NO: 388 - DNA, 24 bp, Artificial Sequence
  primer
gggttcccattactgtcaaatgga

SEQ ID NO: 389 - DNA, 28 bp, Artificial Sequence
  primer
agcgttatgtgacgaagtgaatatttct

SEQ ID NO: 390 - DNA, 19 bp, Artificial Sequence
  primer
tggtgttggtggttgtggt

SEQ ID NO: 391 - DNA, 28 bp, Artificial Sequence
  primer
agcgttatgtgacgaagtgaatatttct

SEQ ID NO: 392 - DNA, 28 bp, Artificial Sequence
  primer
ctgttctgctatttgatgaaaccgtttt

SEQ ID NO: 393 - DNA, 28 bp, Artificial Sequence
  primer
agcgttatgtgacgaagtgaatatttct

SEQ ID NO: 394 - DNA, 24 bp, Artificial Sequence
  primer
ttcggttgttggtttcaggtctaa

SEQ ID NO: 395 - DNA, 28 bp, Artificial Sequence
  primer
agcgttatgtgacgaagtgaatatttct

SEQ ID NO: 396 - DNA, 28 bp, Artificial Sequence
  primer
cctagtgtacccataagcaactcttcta

SEQ ID NO: 397 - DNA, 21 bp, Artificial Sequence
  primer
acctacatcccaccacagagt

SEQ ID NO: 398 - DNA, 25 bp, Artificial Sequence
  primer
gcttacaaccttagacacagacaca

SEQ ID NO: 399 - DNA, 21 bp, Artificial Sequence
  primer
acctacatcccaccacagagt

SEQ ID NO: 400 - DNA, 24 bp, Artificial Sequence
  primer
acgaagcctaaacaccctgtattg

SEQ ID NO: 401 - DNA, 27 bp, Artificial Sequence
  primer
tgcttatgggtacactaggtattgtgt

SEQ ID NO: 402 - DNA, 24 bp, Artificial Sequence
  primer
gggttcccattactgtcaaatgga

SEQ ID NO: 403 - DNA, 27 bp, Artificial Sequence
  primer
tgcttatgggtacactaggtattgtgt

SEQ ID NO: 404 - DNA, 19 bp, Artificial Sequence
  primer
tggtgttggtggttgtggt

SEQ ID NO: 405 - DNA, 27 bp, Artificial Sequence
  primer
tgcttatgggtacactaggtattgtgt

SEQ ID NO: 406 - DNA, 25 bp, Artificial Sequence
  primer
gcttacaaccttagacacagacaca

SEQ ID NO: 407 - DNA, 27 bp, Artificial Sequence
  primer
tgcttatgggtacactaggtattgtgt

SEQ ID NO: 408 - DNA, 24 bp, Artificial Sequence
  primer
acgaagcctaaacaccctgtattg

SEQ ID NO: 409 - DNA, 22 bp, Artificial Sequence
  primer
ccggacagtggatatggcaata

SEQ ID NO: 410 - DNA, 28 bp, Artificial Sequence
  primer
catcatttagtgcatatacaggattccc

SEQ ID NO: 411 - DNA, 22 bp, Artificial Sequence
  primer
ccggacagtggatatggcaata

SEQ ID NO: 412 - DNA, 21 bp, Artificial Sequence
  primer
gggtgttcgatagctgttcaa

SEQ ID NO: 413 - DNA, 23 bp, Artificial Sequence
  primer
cctgcaatacgtctatgcacaat

SEQ ID NO: 414 - DNA, 28 bp, Artificial Sequence
  primer
catcatttagtgcatatacaggattccc

SEQ ID NO: 415 - DNA, 23 bp, Artificial Sequence
  primer
cctgcaatacgtctatgcacaat

SEQ ID NO: 416 - DNA, 21 bp, Artificial Sequence
  primer
gggtgttcgatagctgttcaa

SEQ ID NO: 417 - DNA, 23 bp, Artificial Sequence
  primer
cctgcaatacgtctatgcacaat

SEQ ID NO: 418 - DNA, 24 bp, Artificial Sequence
  primer
ttttttgtcgtccaccaccttttg

SEQ ID NO: 419 - DNA, 23 bp, Artificial Sequence
  primer
cctgcaatacgtctatgcacaat

SEQ ID NO: 420 - DNA, 27 bp, Artificial Sequence
  primer
tccaacactatgtcctttaattgtggt

SEQ ID NO: 421 - DNA, 23 bp, Artificial Sequence
  primer
cctgcaatacgtctatgcacaat

SEQ ID NO: 422 - DNA, 27 bp, Artificial Sequence
  primer
ccagtaacatttgctgaaatatgcgaa

SEQ ID NO: 423 - DNA, 21 bp, Artificial Sequence
  primer
tgcgaccaccaaatacactgt

SEQ ID NO: 424 - DNA, 21 bp, Artificial Sequence
  primer
gtgttgacaatgcgtgacact

SEQ ID NO: 425 - DNA, 20 bp, Artificial Sequence
  primer
cgtggtgtgcgaccaactaa

SEQ ID NO: 426 - DNA, 28 bp, Artificial Sequence
  primer
catcatttagtgcatatacaggattccc

SEQ ID NO: 427 - DNA, 20 bp, Artificial Sequence
  primer
cgtggtgtgcgaccaactaa

SEQ ID NO: 428 - DNA, 21 bp, Artificial Sequence
  primer
gggtgttcgatagctgttcaa

SEQ ID NO: 429 - DNA, 20 bp, Artificial Sequence
  primer
cgtggtgtgcgaccaactaa

SEQ ID NO: 430 - DNA, 21 bp, Artificial Sequence
  primer
gtgttgacaatgcgtgacact

SEQ ID NO: 431 - DNA, 22 bp, Artificial Sequence
  primer
gcgggtatggcaatactgaagt

SEQ ID NO: 432 - DNA, 26 bp, Artificial Sequence
  primer
tggtgtttggcatatagtgtgtcttt

SEQ ID NO: 433 - DNA, 25 bp, Artificial Sequence
  primer
cacagagctgcaaacaactatacat

SEQ ID NO: 434 - DNA, 27 bp, Artificial Sequence
  primer
cacatacagcatatggattcccatctc

SEQ ID NO: 435 - DNA, 27 bp, Artificial Sequence
  primer
ggatgtaaagcatagaccattggtaca

SEQ ID NO: 436 - DNA, 28 bp, Artificial Sequence
  primer
gttttcgtcaaatggaaactcattagga

SEQ ID NO: 437 - DNA, 28 bp, Artificial Sequence
  primer
cggaaatccagtgtatgagcttaatgat

SEQ ID NO: 438 - DNA, 26 bp, Artificial Sequence
  primer
tgacacacatttaaacgttggcaaag

SEQ ID NO: 439 - DNA, 19 bp, Artificial Sequence
  primer
catgcgggtggtcaggtaa

SEQ ID NO: 440 - DNA, 19 bp, Artificial Sequence
  primer
aaggcgacggctttggtat

SEQ ID NO: 441 - DNA, 23 bp, Artificial Sequence
  primer
gctcacacaaaggacggattaac

SEQ ID NO: 442 - DNA, 22 bp, Artificial Sequence
  primer
ccaatgccatgtagacgacact

SEQ ID NO: 443 - DNA, 28 bp, Artificial Sequence
  primer
ggaacaacattagaacagcaatacaaca

SEQ ID NO: 444 - DNA, 26 bp, Artificial Sequence
  primer
tgtccagatgtctttgcttttcttca

SEQ ID NO: 445 - DNA, 17 bp, Artificial Sequence
  primer
cggtggaccggtcgatg

SEQ ID NO: 446 - DNA, 24 bp, Artificial Sequence
  primer
tcagttgtctctggttgcaaatct

SEQ ID NO: 447 - DNA, 24 bp, Artificial Sequence
  primer
cctatagttccagggtctccacaa

SEQ ID NO: 448 - DNA, 26 bp, Artificial Sequence
  primer
atccgtgcttacaaccttagatactg

SEQ ID NO: 449 - DNA, 26 bp, Artificial Sequence
  primer
ctcagaggaggaggatgaaatagatg

SEQ ID NO: 450 - DNA, 26 bp, Artificial Sequence
  primer
ccattaacaggtcttccaaagtacga

SEQ ID NO: 451 - DNA, 21 bp, Artificial Sequence
  primer
ggaattgtgtgccccatctgt

SEQ ID NO: 452 - DNA, 22 bp, Artificial Sequence
  primer
catccattacatcccgtaccct

SEQ ID NO: 453 - DNA, 24 bp, Artificial Sequence
  primer
tcagatagtggctatggctgttct

SEQ ID NO: 454 - DNA, 20 bp, Artificial Sequence
  primer
ccgttgtctatagcctccgt

SEQ ID NO: 455 - DNA, 28 bp, Artificial Sequence
  primer
ttcactgcaagacatagaaataacctgt

SEQ ID NO: 456 - DNA, 26 bp, Artificial Sequence
  primer
ctatacatttatggcatgcagcatgg

SEQ ID NO: 457 - DNA, 31 bp, Artificial Sequence
  primer
ctaaaatgtcctccaatactactaaccacaa

SEQ ID NO: 458 - DNA, 28 bp, Artificial Sequence
  primer
gtcatttatttcatatactggattgcca

SEQ ID NO: 459 - DNA, 22 bp, Artificial Sequence
  primer
ggattggacactgcaagacaca

SEQ ID NO: 460 - DNA, 28 bp, Artificial Sequence
  primer
cccatgctacataggtcatacaattgtc

SEQ ID NO: 461 - DNA, 20 bp, Artificial Sequence
  primer
tgacgacacggtatccgcta

SEQ ID NO: 462 - DNA, 18 bp, Artificial Sequence
  primer
acgtctggccgtaggtct

SEQ ID NO: 463 - DNA, 22 bp, Artificial Sequence
  primer
cagctacacctacaggcaacaa

SEQ ID NO: 464 - DNA, 24 bp, Artificial Sequence
  primer
gtcgctatgttttcgcaatctgta

SEQ ID NO: 465 - DNA, 27 bp, Artificial Sequence
  primer
cgaaaacatagcgaccactatagagat

SEQ ID NO: 466 - DNA, 27 bp, Artificial Sequence
  primer
ttgtacactatctggaattgcaacagt

SEQ ID NO: 467 - DNA, 25 bp, Artificial Sequence
  primer
tcagactctgtgtatggagacacat

SEQ ID NO: 468 - DNA, 22 bp, Artificial Sequence
  primer
cccagctatgttgtgaaatcgt

SEQ ID NO: 469 - DNA, 25 bp, Artificial Sequence
  primer
tcctaagaaacgtaaacgtgttccc

SEQ ID NO: 470 - DNA, 27 bp, Artificial Sequence
  primer
gtatttacaactcttgccacagaagga

SEQ ID NO: 471 - DNA, 27 bp, Artificial Sequence
  primer
gcatattttatcatgctggcagctcta

SEQ ID NO: 472 - DNA, 28 bp, Artificial Sequence
  primer
tcaggtaactgcaccctaaatactctat

SEQ ID NO: 473 - DNA, 28 bp, Artificial Sequence
  primer
cagaggaagaaaacgatgaaatagatgg

SEQ ID NO: 474 - DNA, 21 bp, Artificial Sequence
  primer
agaaacagctgctggaatgct

SEQ ID NO: 475 - DNA, 20 bp, Artificial Sequence
  primer
tgcatcccagcagtaagcaa

SEQ ID NO: 476 - DNA, 28 bp, Artificial Sequence
  primer
ctcgtcatctgatattacatctcctgtt

SEQ ID NO: 477 - DNA, 22 bp, Artificial Sequence
  primer
gcgggtatggcaatactgaagt

SEQ ID NO: 478 - DNA, 27 bp, Artificial Sequence
  primer
tggagtttcattctctcgttcactatg

SEQ ID NO: 479 - DNA, 23 bp, Artificial Sequence
  primer
cggcattggaaataccctacgat

SEQ ID NO: 480 - DNA, 28 bp, Artificial Sequence
  primer
tcttaaacattttgtacacactccgtgt

SEQ ID NO: 481 - DNA, 23 bp, Artificial Sequence
  primer
cactagatggcaaccctgtatct

SEQ ID NO: 482 - DNA, 28 bp, Artificial Sequence
  primer
aatgtaaaaaccaccagtctgctatgta

SEQ ID NO: 483 - DNA, 28 bp, Artificial Sequence
  primer
ctggtggtttttacatttccaaatccat

SEQ ID NO: 484 - DNA, 26 bp, Artificial Sequence
  primer
cgttgagaaagagtctccatcgtttt

SEQ ID NO: 485 - DNA, 19 bp, Artificial Sequence
  primer
catgcgggtggtcaggtaa

SEQ ID NO: 486 - DNA, 24 bp, Artificial Sequence
  primer
gaattcgatgtggtggtgttgttg

SEQ ID NO: 487 - DNA, 19 bp, Artificial Sequence
  primer
cagctgcatgcacaaacca

SEQ ID NO: 488 - DNA, 28 bp, Artificial Sequence
  primer
gccatgtagatgacacttgttcatacaa

SEQ ID NO: 489 - DNA, 32 bp, Artificial Sequence
  primer
ggaacaacattagaaaaattgacaaacaaagg

SEQ ID NO: 490 - DNA, 24 bp, Artificial Sequence
  primer
ttttcttctggacacaacggtctt

SEQ ID NO: 491 - DNA, 25 bp, Artificial Sequence
  primer
gaaacgattccacaacataggagga

SEQ ID NO: 492 - DNA, 25 bp, Artificial Sequence
  primer
acatagtcttgcaacgtaggtgttt

SEQ ID NO: 493 - DNA, 26 bp, Artificial Sequence
  primer
gccacaagtgtctatttttgttgatg

SEQ ID NO: 494 - DNA, 23 bp, Artificial Sequence
  primer
tttagacactgggacaggtggta

SEQ ID NO: 495 - DNA, 26 bp, Artificial Sequence
  primer
cagatgaggaggatgtcatagacagt

SEQ ID NO: 496 - DNA, 28 bp, Artificial Sequence
  primer
cattaacagctcttgcaatatgcgaata

SEQ ID NO: 497 - DNA, 19 bp, Artificial Sequence
  primer
aatcgtgtgccccaactgt

SEQ ID NO: 498 - DNA, 24 bp, Artificial Sequence
  primer
cccctgtctgtctgtcaattactg

SEQ ID NO: 499 - DNA, 25 bp, Artificial Sequence
  primer
gatgagctagaagacagcggatatg

SEQ ID NO: 500 - DNA, 20 bp, Artificial Sequence
  primer
catcccccaccccactagat

SEQ ID NO: 501 - DNA, 28 bp, Artificial Sequence
  primer
agcattggagacaactatacacaacatt

SEQ ID NO: 502 - DNA, 28 bp, Artificial Sequence
  primer
cgcaaacacagtttacatattccaaatg

SEQ ID NO: 503 - DNA, 28 bp, Artificial Sequence
  primer
tgtgaaacatagggcattagtgcaatta

SEQ ID NO: 504 - DNA, 28 bp, Artificial Sequence
  primer
catacactgggttaccattttcatcaaa

SEQ ID NO: 505 - DNA, 28 bp, Artificial Sequence
  primer
ggatgctgcaaagtattctaaaacacaa

SEQ ID NO: 506 - DNA, 21 bp, Artificial Sequence
  primer
gtggtggtcggttatcgttgt

SEQ ID NO: 507 - DNA, 23 bp, Artificial Sequence
  primer
acgtactgcaactaactgcacaa

SEQ ID NO: 508 - DNA, 27 bp, Artificial Sequence
  primer
gccaggtggatgacatagaactataca

SEQ ID NO: 509 - DNA, 33 bp, Artificial Sequence
  primer
attctgtatatggaaatacattagaacaaacag

SEQ ID NO: 510 - DNA, 28 bp, Artificial Sequence
  primer
tcgtttgtttaaatccacatgtcgtttt

SEQ ID NO: 511 - DNA, 27 bp, Artificial Sequence
  primer
cgatttcataatatttcgggtcgttgg

SEQ ID NO: 512 - DNA, 26 bp, Artificial Sequence
  primer
catattcctttaacgttggcttgtgt

SEQ ID NO: 513 - DNA, 23 bp, Artificial Sequence
  primer
ttgttgtagacggtgctgacttt

SEQ ID NO: 514 - DNA, 27 bp, Artificial Sequence
  primer
atcagtgctgacaactttagatacagg

SEQ ID NO: 515 - DNA, 19 bp, Artificial Sequence
  primer
gtgccctacctgtgcacaa

SEQ ID NO: 516 - DNA, 27 bp, Artificial Sequence
  primer
ttcttctctctatgactgcttctacct

SEQ ID NO: 517 - DNA, 26 bp, Artificial Sequence
  primer
attatttgaactaccagacagcggtt

SEQ ID NO: 518 - DNA, 27 bp, Artificial Sequence
  primer
gctactagaggttatactatccccact

SEQ ID NO: 519 - DNA, 27 bp, Artificial Sequence
  primer
cgaggtagaagaaagcatccatgaaat

SEQ ID NO: 520 - DNA, 23 bp, Artificial Sequence
  primer
catactccatatggctggccttc

SEQ ID NO: 521 - DNA, 27 bp, Artificial Sequence
  primer
cattagtgcaattaaaatgcccacctt

SEQ ID NO: 522 - DNA, 28 bp, Artificial Sequence
  primer
tcattgtgaaatgtaaagaccactaccc

SEQ ID NO: 523 - DNA, 24 bp, Artificial Sequence
  primer
ggaaacccagtgtatgggcttaat

SEQ ID NO: 524 - DNA, 26 bp, Artificial Sequence
  primer
ggaaagcgtctccatcattttctttg

SEQ ID NO: 525 - DNA, 26 bp, Artificial Sequence
  primer
aaaatatatgggaagtgcatgtgggt

SEQ ID NO: 526 - DNA, 20 bp, Artificial Sequence
  primer
gctttggtatgggtctcggt

SEQ ID NO: 527 - DNA, 26 bp, Artificial Sequence
  primer
tctacatctgactgcacaaacaaaga

SEQ ID NO: 528 - DNA, 27 bp, Artificial Sequence
  primer
ccatctccatgtagatgaagcatcttg

SEQ ID NO: 529 - DNA, 28 bp, Artificial Sequence
  primer
ggagaaacgttagaaaaacaatgcaaca

SEQ ID NO: 530 - DNA, 25 bp, Artificial Sequence
  primer
tccaccgatgttatggaatcgtttt

SEQ ID NO: 531 - DNA, 26 bp, Artificial Sequence
  primer
gggtgacttttatttacaccctagtt

SEQ ID NO: 532 - DNA, 26 bp, Artificial Sequence
  primer
catcagtgctaacaaccttagacact

SEQ ID NO: 533 - DNA, 25 bp, Artificial Sequence
  primer
catctactatcatgcaggcagttct

SEQ ID NO: 534 - DNA, 27 bp, Artificial Sequence
  primer
actctgtattgcaaaccagataccttg

SEQ ID NO: 535 - DNA, 23 bp, Artificial Sequence
  primer
cggctgttcacagagagcataat

SEQ ID NO: 536 - DNA, 25 bp, Artificial Sequence
  primer
cccgtacgtctactaactactgctt

SEQ ID NO: 537 - DNA, 20 bp, Artificial Sequence
  primer
ggtgtattccgtgccagaca

SEQ ID NO: 538 - DNA, 19 bp, Artificial Sequence
  primer
gtacactgccgccatgttc

SEQ ID NO: 539 - DNA, 25 bp, Artificial Sequence
  primer
caccaccttgcaggacattacaata

SEQ ID NO: 540 - DNA, 20 bp, Artificial Sequence
  primer
gattggcatgcagctagtgg

SEQ ID NO: 541 - DNA, 31 bp, Artificial Sequence
  primer
attagatgggtatgcaataagtttagatagg

SEQ ID NO: 542 - DNA, 28 bp, Artificial Sequence
  primer
ctgttttggtcaaatggaaatgcattag

SEQ ID NO: 543 - DNA, 22 bp, Artificial Sequence
  primer
cacagtaacagtacaggccaca

SEQ ID NO: 544 - DNA, 25 bp, Artificial Sequence
  primer
cgtatccaatgccaggtacatgaaa

SEQ ID NO: 545 - DNA, 20 bp, Artificial Sequence
  primer
ctcggactcggtgtatgcaa

SEQ ID NO: 546 - DNA, 27 bp, Artificial Sequence
  primer
ctgtcctgtatagcttcctgctatttt

SEQ ID NO: 547 - DNA, 25 bp, Artificial Sequence
  primer
gcaataaccattcagggttccaatt

SEQ ID NO: 548 - DNA, 22 bp, Artificial Sequence
  primer
agtattgacaaccttcgccaca

SEQ ID NO: 549 - DNA, 26 bp, Artificial Sequence
  primer
catgcagttaatcaccaacatcaact

SEQ ID NO: 550 - DNA, 23 bp, Artificial Sequence
  primer
tgctgtagttgtcgcagagtatc

SEQ ID NO: 551 - DNA, 18 bp, Artificial Sequence
  primer
cgtggtgtgcaactgcaa

SEQ ID NO: 552 - DNA, 23 bp, Artificial Sequence
  primer
cactgtgtcgcctgtttgtttat

SEQ ID NO: 553 - DNA, 24 bp, Artificial Sequence
  primer
tcagatagtggctatggctgttct

SEQ ID NO: 554 - DNA, 25 bp, Artificial Sequence
  primer
actatccccaccactactttgtgta

SEQ ID NO: 555 - DNA, 26 bp, Artificial Sequence
  primer
ctacaagacgtatctattgcctgtgt

SEQ ID NO: 556 - DNA, 29 bp, Artificial Sequence
  primer
aagtctatacatttatggcatgcagcata

SEQ ID NO: 557 - DNA, 30 bp, Artificial Sequence
  primer
cattattacagctaaaatgtcctccaatcc

SEQ ID NO: 558 - DNA, 28 bp, Artificial Sequence
  primer
gaaatgcatgtggaaatgtaaataccgt

SEQ ID NO: 559 - DNA, 20 bp, Artificial Sequence
  primer
tgacgacacggtatccgcta

SEQ ID NO: 560 - DNA, 18 bp, Artificial Sequence
  primer
cccacggatgcggttttg

SEQ ID NO: 561 - DNA, 28 bp, Artificial Sequence
  primer
tcctgtgttcaagtacaagtaacaacaa

SEQ ID NO: 562 - DNA, 25 bp, Artificial Sequence
  primer
ggtctgcatatttgcgtagcctata

SEQ ID NO: 563 - DNA, 27 bp, Artificial Sequence
  primer
cgcaaatatgcagaccattactcagaa

SEQ ID NO: 564 - DNA, 24 bp, Artificial Sequence
  primer
cccaccgagatttgtacactgtta

SEQ ID NO: 565 - DNA, 27 bp, Artificial Sequence
  primer
aaactctgtatatggagagacactgga

SEQ ID NO: 566 - DNA, 27 bp, Artificial Sequence
  primer
cgtttgtccttaaggtgtctacgtttt

SEQ ID NO: 567 - DNA, 28 bp, Artificial Sequence
  primer
gcacacaatattatttatggccatggta

SEQ ID NO: 568 - DNA, 19 bp, Artificial Sequence
  primer
gctgacaactctggccaca

SEQ ID NO: 569 - DNA, 24 bp, Artificial Sequence
  primer
ggagttagtcatgcacaactacca

SEQ ID NO: 570 - DNA, 25 bp, Artificial Sequence
  primer
tcaaaaacagctgctgtagtgttct

SEQ ID NO: 571 - DNA, 21 bp, Artificial Sequence
  primer
agcaccttgtcctttgtgtgt

SEQ ID NO: 572 - DNA, 28 bp, Artificial Sequence
  primer
caattgtttctacaaagaaccagccatt

SEQ ID NO: 573 - DNA, 21 bp, Artificial Sequence
  primer
cggacagcggatatggcaata

SEQ ID NO: 574 - DNA, 26 bp, Artificial Sequence
  primer
tctgttgtttccacatccataacact

SEQ ID NO: 575 - DNA, 27 bp, Artificial Sequence
  primer
ctgcatgaattatgtgaagctttgaac

SEQ ID NO: 576 - DNA, 29 bp, Artificial Sequence
  primer
gtaaacattgtttgcatactgcatatgga

SEQ ID NO: 577 - DNA, 31 bp, Artificial Sequence
  primer
agtatgtccaccattactaataacgtcaaac

SEQ ID NO: 578 - DNA, 28 bp, Artificial Sequence
  primer
tcattcaatgtatacacagcattcccat

SEQ ID NO: 579 - DNA, 24 bp, Artificial Sequence
  primer
gcacaacagtgggaggtctatatg

SEQ ID NO: 580 - DNA, 18 bp, Artificial Sequence
  primer
ccacgcaggtggtaaggg

SEQ ID NO: 581 - DNA, 21 bp, Artificial Sequence
  primer
ctaacactggagggcaccaaa

SEQ ID NO: 582 - DNA, 26 bp, Artificial Sequence
  primer
atgccaggttgaggatacgtttttat

SEQ ID NO: 583 - DNA, 28 bp, Artificial Sequence
  primer
gagagtatagacgttatagcaggtctgt

SEQ ID NO: 584 - DNA, 24 bp, Artificial Sequence
  primer
tcccgctatttcatggaacctttt

SEQ ID NO: 585 - DNA, 26 bp, Artificial Sequence
  primer
ggccctatacacatttactacgcaaa

SEQ ID NO: 586 - DNA, 21 bp, Artificial Sequence
  primer
caattcgagacacaggtgcag

SEQ ID NO: 587 - DNA, 20 bp, Artificial Sequence
  primer
gcgtgaccagctaccagaaa

SEQ ID NO: 588 - DNA, 21 bp, Artificial Sequence
  primer
catctgctgtacaacgcgaag

SEQ ID NO: 589 - DNA, 20 bp, Artificial Sequence
  primer
gggcgaactaagcctggttt

SEQ ID NO: 590 - DNA, 27 bp, Artificial Sequence
  primer
ctcatcatccgaaacattatctcctgt

SEQ ID NO: 591 - DNA, 24 bp, Artificial Sequence
  primer
caaaccatgtcacgtagaagacag

SEQ ID NO: 592 - DNA, 19 bp, Artificial Sequence
  primer
ccccaccccacttgattga

SEQ ID NO: 593 - DNA, 24 bp, Artificial Sequence
  primer
agaatcggtgcatgaaataaggct

SEQ ID NO: 594 - DNA, 27 bp, Artificial Sequence
  primer
cacacgccatatggattattgtctcta

SEQ ID NO: 595 - DNA, 27 bp, Artificial Sequence
  primer
ccttagtacaaataaaatgcccaccat

SEQ ID NO: 596 - DNA, 28 bp, Artificial Sequence
  primer
gggtttttgaaatgaaacacaaccaatc

SEQ ID NO: 597 - DNA, 27 bp, Artificial Sequence
  primer
gtaacaggagtatgggaagtacatgtg

SEQ ID NO: 598 - DNA, 19 bp, Artificial Sequence
  primer
gcggaggtcttggaggttt

SEQ ID NO: 599 - DNA, 21 bp, Artificial Sequence
  primer
tcactgcaactgagtgcacaa

SEQ ID NO: 600 - DNA, 28 bp, Artificial Sequence
  primer
tgccaggtagatgaaatttgaacataca

SEQ ID NO: 601 - DNA, 27 bp, Artificial Sequence
  primer
gtatgggaaaacattagaagagagggt

SEQ ID NO: 602 - DNA, 27 bp, Artificial Sequence
  primer
cgcttgtttgcattaacatgtctttct

SEQ ID NO: 603 - DNA, 27 bp, Artificial Sequence
  primer
gacatgttaatgcaaacaagcgatttc

SEQ ID NO: 604 - DNA, 28 bp, Artificial Sequence
  primer
tcagttgtttcaggttgcagatctaata

SEQ ID NO: 605 - DNA, 22 bp, Artificial Sequence
  primer
ttttactacgtcgcaggcgtaa

SEQ ID NO: 606 - DNA, 26 bp, Artificial Sequence
  primer
tgcttacaaccttagagacaggtaca

SEQ ID NO: 607 - DNA, 28 bp, Artificial Sequence
  primer
aagcatctattattatgcaggcagttct

SEQ ID NO: 608 - DNA, 20 bp, Artificial Sequence
  primer
cctgtattgcaggccagaca

SEQ ID NO: 609 - DNA, 24 bp, Artificial Sequence
  primer
gatgaggaggatacagatggtgtg

SEQ ID NO: 610 - DNA, 24 bp, Artificial Sequence
  primer
gcatttgctgtagagtacgaaggt

SEQ ID NO: 611 - DNA, 22 bp, Artificial Sequence
  primer
gctgttgggcacattacaagtt

SEQ ID NO: 612 - DNA, 28 bp, Artificial Sequence
  primer
tcctctgaaatgttatctcctgtttgtt

SEQ ID NO: 613 - DNA, 22 bp, Artificial Sequence
  primer
caagacagcgggtatggcaata

SEQ ID NO: 614 - DNA, 25 bp, Artificial Sequence
  primer
ggtactgttttgtgagcctccattt

SEQ ID NO: 615 - DNA, 25 bp, Artificial Sequence
  primer
gcaccacttgagtgaggtattagaa

SEQ ID NO: 616 - DNA, 28 bp, Artificial Sequence
  primer
acaataaacatactctgcacactgcata

SEQ ID NO: 617 - DNA, 29 bp, Artificial Sequence
  primer
agaatgttagtgtttcagtttcaaaatcc

SEQ ID NO: 618 - DNA, 26 bp, Artificial Sequence
  primer
ttttctttgtcctcgtcgttatccaa

SEQ ID NO: 619 - DNA, 23 bp, Artificial Sequence
  primer
acaacaaccaccctggtgataag

SEQ ID NO: 620 - DNA, 28 bp, Artificial Sequence
  primer
tattgtctgtacttgtccaatgatatgt

SEQ ID NO: 621 - DNA, 27 bp, Artificial Sequence
  primer
tcagtgtatggagctacactagaaagt

SEQ ID NO: 622 - DNA, 24 bp, Artificial Sequence
  primer
caattgcttttcctccggagttaa

SEQ ID NO: 623 - DNA, 27 bp, Artificial Sequence
  primer
tgcattgtgacagaaaaagacgatttc

SEQ ID NO: 624 - DNA, 19 bp, Artificial Sequence
  primer
acgtcttgcagcgttggta

SEQ ID NO: 625 - DNA, 23 bp, Artificial Sequence
  primer
agggatcctcctttgcattatgg

SEQ ID NO: 626 - DNA, 24 bp, Artificial Sequence
  primer
acaacctttgaaacaggtgttgga

SEQ ID NO: 627 - DNA, 20 bp, Artificial Sequence
  primer
atcatgcaggcagttcacga

SEQ ID NO: 628 - DNA, 28 bp, Artificial Sequence
  primer
caaccgtaccctaaataccctatattga

SEQ ID NO: 629 - DNA, 25 bp, Artificial Sequence
  primer
acagcaagctagacaagctaaacaa

SEQ ID NO: 630 - DNA, 20 bp, Artificial Sequence
  primer
tgtacaacacgcaggtcctc

SEQ ID NO: 631 - DNA, 23 bp, Artificial Sequence
  primer
gttaacagtaacgtgcccactct

SEQ ID NO: 632 - DNA, 28 bp, Artificial Sequence
  primer
ttctacaattgcctctacttcaaaccat

SEQ ID NO: 633 - DNA, 28 bp, Artificial Sequence
  primer
aaaattattgagctagaagacagcggat

SEQ ID NO: 634 - DNA, 25 bp, Artificial Sequence
  primer
ccccactagactccgagtcatttaa

SEQ ID NO: 635 - DNA, 20 bp, Artificial Sequence
  primer
gtcaggcgttggagacatct

SEQ ID NO: 636 - DNA, 27 bp, Artificial Sequence
  primer
tcgtaagcacactttacatactgcaaa

SEQ ID NO: 637 - DNA, 30 bp, Artificial Sequence
  primer
attagatggtaacgacatttcaatagatgt

SEQ ID NO: 638 - DNA, 31 bp, Artificial Sequence; identical to SEQ ID NO: 280
  primer

SEQ ID NO: 639 - DNA, 26 bp, Artificial Sequence
  primer
acaattatgggaggtacatgtgggta

SEQ ID NO: 640 - DNA, 23 bp, Artificial Sequence
  primer
ccctgtgtactttcgttgttggt

SEQ ID NO: 641 - DNA, 27 bp, Artificial Sequence
  primer
gaggaggactacacagtacaactaact

SEQ ID NO: 642 - DNA, 27 bp, Artificial Sequence
  primer
ccaatgccatgtggatgacatattaca

SEQ ID NO: 643 - DNA, 29 bp, Artificial Sequence
  primer
cgctatatggagacacattagaacaaaca

SEQ ID NO: 644 - DNA, 28 bp, Artificial Sequence
  primer
cgacccgaaatattatgaaaccttttgt

SEQ ID NO: 645 - DNA, 28 bp, Artificial Sequence
  primer
ctgattttatgttgcaccctagctattt

SEQ ID NO: 646 - DNA, 23 bp, Artificial Sequence
  primer
gcttacaaccttagacacaggca

SEQ ID NO: 647 - DNA, 23 bp, Artificial Sequence
  primer
tgcttatgggcacatgtaccatt

SEQ ID NO: 648 - DNA, 27 bp, Artificial Sequence
  primer
ctgttcttcgttctattaccgcttcta

SEQ ID NO: 649 - DNA, 27 bp, Artificial Sequence
  primer
aaagaaggttaataacagtgccagaca

SEQ ID NO: 650 - DNA, 26 bp, Artificial Sequence
  primer
gtctatttgactgtcgctacaaacac

SEQ ID NO: 651 - DNA, 25 bp, Artificial Sequence
  primer
gcatcaattgtgtgttttgcaaagg

SEQ ID NO: 652 - DNA, 22 bp, Artificial Sequence
  primer
gcatttcagacacgctgcatac

SEQ ID NO: 653 - DNA, 28 bp, Artificial Sequence
  primer
agatagaaagcataggcacctagtacaa

SEQ ID NO: 654 - DNA, 31 bp, Artificial Sequence; identical to SEQ ID NO: 306
  primer

SEQ ID NO: 655 - DNA, 19 bp, Artificial Sequence
  primer
tccgtttgcatccaggcaa

SEQ ID NO: 656 - DNA, 28 bp, Artificial Sequence
  primer
ccaatgccaggtagaggaaatattttca

SEQ ID NO: 657 - DNA, 23 bp, Artificial Sequence
  primer
cctcgtaaacgtaaacgtgttcc

SEQ ID NO: 658 - DNA, 25 bp, Artificial Sequence
  primer
tgacatactcatcagtgctgacaac

SEQ ID NO: 659 - DNA, 28 bp, Artificial Sequence
  primer
gtatgtcacccgtaccagtattttctac

SEQ ID NO: 660 - DNA, 27 bp, Artificial Sequence
  primer
gccaaatttattgggatcaggtaactt

SEQ ID NO: 661 - DNA, 28 bp, Artificial Sequence
  primer
cagatggagttaatcatcctttgctact

SEQ ID NO: 662 - DNA, 19 bp, Artificial Sequence
  primer
tgtaaggctcgcaatccgt

SEQ ID NO: 663 - DNA, 25 bp, Artificial Sequence
  primer
actatcctttgtgtgtcctttgtgt

SEQ ID NO: 664 - DNA, 28 bp, Artificial Sequence
  primer
cgtcatctgaaattttgtcacctgtttt

SEQ ID NO: 665 - DNA, 22 bp, Artificial Sequence
  primer
gaagacagcgggtatggcaata

SEQ ID NO: 666 - DNA, 27 bp, Artificial Sequence
  primer
gataccgagtgctcactacaattactg

SEQ ID NO: 667 - DNA, 23 bp, Artificial Sequence
  primer
caccatctgagcgaggtattaca

SEQ ID NO: 668 - DNA, 31 bp, Artificial Sequence
  primer
acaataaacataccctacatactgcatatgg

SEQ ID NO: 669 - DNA, 23 bp, Artificial Sequence
  primer
gtgggtggtgtaaagtgtcatca

SEQ ID NO: 670 - DNA, 26 bp, Artificial Sequence
  primer
ggacagtaaatactctcggtttccat

SEQ ID NO: 671 - DNA, 23 bp, Artificial Sequence
  primer
gtatcaacacacaaagccactgt

SEQ ID NO: 672 - DNA, 29 bp, Artificial Sequence
  primer
tctgtacttgtccaatgatatgttgttgt

SEQ ID NO: 673 - DNA, 31 bp, Artificial Sequence
  primer
gggcaacattagaaagtataactaaaaaaca

SEQ ID NO: 674 - DNA, 28 bp, Artificial Sequence
  primer
gaaatcgtcttttatgttcacagtgcaa

SEQ ID NO: 675 - DNA, 24 bp, Artificial Sequence
  primer
gctacatttgcactatggcctgta

SEQ ID NO: 676 - DNA, 24 bp, Artificial Sequence
  primer
acaacctttgaaacaggtgttgga

SEQ ID NO: 677 - DNA, 22 bp, Artificial Sequence
  primer
acagcaagctagacaagctgaa

SEQ ID NO: 678 - DNA, 21 bp, Artificial Sequence
  primer
tgtaccacacgtagctcctct

SEQ ID NO: 679 - DNA, 23 bp, Artificial Sequence
  primer
gttaacagtaacgtgcccactct

SEQ ID NO: 680 - DNA, 28 bp, Artificial Sequence
  primer
ttctacaattgcttctacctgaaaccat

SEQ ID NO: 681 - DNA, 21 bp, Artificial Sequence
  primer
agacaaccggcgtatacagtg

SEQ ID NO: 682 - DNA, 24 bp, Artificial Sequence
  primer
cacactactacagtcctcccgtat

SEQ ID NO: 683 - DNA, 25 bp, Artificial Sequence
  primer
gacattggacactacattgcatgac

SEQ ID NO: 684 - DNA, 22 bp, Artificial Sequence
  primer
gattggcatgcagcaaatggta

SEQ ID NO: 685 - DNA, 30 bp, Artificial Sequence
  primer
cctaatacaaataaagtgtccaccaatgct

SEQ ID NO: 686 - DNA, 28 bp, Artificial Sequence
  primer
ctgttttggtcaaatggaaatgcattag

SEQ ID NO: 687 - DNA, 28 bp, Artificial Sequence
  primer
ggaatcggtgtatgcaactacattagaa

SEQ ID NO: 688 - DNA, 26 bp, Artificial Sequence
  primer
cttcgttttgttgttaggtgccttag

SEQ ID NO: 689 - DNA, 27 bp, Artificial Sequence
  primer
ctagtggaaaatgggacgtgcattata

SEQ ID NO: 690 - DNA, 19 bp, Artificial Sequence
  primer
tcgcggtggtgttctgtag

SEQ ID NO: 691 - DNA, 23 bp, Artificial Sequence
  primer
agtagaagtgcaggccaaaacaa

SEQ ID NO: 692 - DNA, 27 bp, Artificial Sequence
  primer
aagcgttatgtttttgcaacctatacc

SEQ ID NO: 693 - DNA, 28 bp, Artificial Sequence
  primer
tacaacctttgccataactatatatggt

SEQ ID NO: 694 - DNA, 21 bp, Artificial Sequence
  primer
attgacaaccttcgccactga

SEQ ID NO: 695 - DNA, 25 bp, Artificial Sequence
  primer
ccaccaacatctactactagccaga

SEQ ID NO: 696 - DNA, 21 bp, Artificial Sequence
  primer
ctgttgtagtgtccgcaggtt

SEQ ID NO: 697 - DNA, 19 bp, Artificial Sequence
  primer
tccgtggtgtgcaactgaa

SEQ ID NO: 698 - DNA, 28 bp, Artificial Sequence
  primer
gactgtgtcacctgtttgtttatctact

SEQ ID NO: 699 - DNA, 23 bp, Artificial Sequence
  primer
aaacgaagactgtttgaggagca

SEQ ID NO: 700 - DNA, 28 bp, Artificial Sequence
  primer
gacacaatttggttgccttcttcattaa

SEQ ID NO: 701 - DNA, 28 bp, Artificial Sequence
  primer
agcgttatgtgacgaagtgaatatttct

SEQ ID NO: 702 - DNA, 27 bp, Artificial Sequence
  primer
aaaattttaaacacggttgacatacac

SEQ ID NO: 703 - DNA, 29 bp, Artificial Sequence
  primer
caagttaaatgccctccattactgataac

SEQ ID NO: 704 - DNA, 24 bp, Artificial Sequence
  primer
gggttcccattactgtcaaatgga

SEQ ID NO: 705 - DNA, 26 bp, Artificial Sequence
  primer
gggtaaaaggcatatgggaagtacat

SEQ ID NO: 706 - DNA, 19 bp, Artificial Sequence
  primer
tggtgttggtggttgtggt

SEQ ID NO: 707 - DNA, 21 bp, Artificial Sequence
  primer
acctacatcccaccacagagt

SEQ ID NO: 708 - DNA, 24 bp, Artificial Sequence
  primer
gtccaatgccatgttgttgttaca

SEQ ID NO: 709 - DNA, 29 bp, Artificial Sequence
  primer
agacaatcagtatatggcactacgttaga

SEQ ID NO: 710 - DNA, 28 bp, Artificial Sequence
  primer
ctgttctgctatttgatgaaaccgtttt

SEQ ID NO: 711 - DNA, 28 bp, Artificial Sequence
  primer
tgggtcaggttttatattacaccctagt

SEQ ID NO: 712 - DNA, 25 bp, Artificial Sequence
  primer
gcttacaaccttagacacagacaca

SEQ ID NO: 713 - DNA, 20 bp, Artificial Sequence
  primer
tgcaggtagcacacgtttgt

SEQ ID NO: 714 - DNA, 24 bp, Artificial Sequence
  primer
acgaagcctaaacaccctgtattg

SEQ ID NO: 715 - DNA, 25 bp, Artificial Sequence
  primer
actcagaggatgaggatgaaacaga

SEQ ID NO: 716 - DNA, 28 bp, Artificial Sequence
  primer
cctagtgtacccataagcaactcttcta

SEQ ID NO: 717 - DNA, 27 bp, Artificial Sequence
  primer
tgcttatgggtacactaggtattgtgt

SEQ ID NO: 718 - DNA, 26 bp, Artificial Sequence
  primer
tggaattggatcccctgtttttcttt

SEQ ID NO: 719 - DNA, 22 bp, Artificial Sequence
  primer
ccggacagtggatatggcaata

SEQ ID NO: 720 - DNA, 25 bp, Artificial Sequence
  primer
ggtctatctctgtacttctgtcgct

SEQ ID NO: 721 - DNA, 23 bp, Artificial Sequence
  primer
cctgcaatacgtctatgcacaat

SEQ ID NO: 722 - DNA, 23 bp, Artificial Sequence
  primer
catgctgcatatggcgtattgtc

SEQ ID NO: 723 - DNA, 20 bp, Artificial Sequence
  primer
acacagaagcctgctgcaaa

SEQ ID NO: 724 - DNA, 28 bp, Artificial Sequence
  primer
catcatttagtgcatatacaggattccc

SEQ ID NO: 725 - DNA, 20 bp, Artificial Sequence
  primer
gggcacaacaatgggaggta

SEQ ID NO: 726 - DNA, 21 bp, Artificial Sequence
  primer
gggtgttcgatagctgttcaa

SEQ ID NO: 727 - DNA, 21 bp, Artificial Sequence
  primer
tgcgaccaccaaatacactgt

SEQ ID NO: 728 - DNA, 28 bp, Artificial Sequence
  primer
caatgccaggtagatgacacttctttaa

SEQ ID NO: 729 - DNA, 25 bp, Artificial Sequence
  primer
gtaggtctgtgtatggtgctacatt

SEQ ID NO: 730 - DNA, 24 bp, Artificial Sequence
  primer
ttttttgtcgtccaccaccttttg

SEQ ID NO: 731 - DNA, 25 bp, Artificial Sequence
  primer
gggattactactttgtggccgtata

SEQ ID NO: 732 - DNA, 21 bp, Artificial Sequence
  primer
gtgttgacaatgcgtgacact

SEQ ID NO: 733 - DNA, 28 bp, Artificial Sequence
  primer
ggaggatgaagtagataatatgcgtgac

SEQ ID NO: 734 - DNA, 27 bp, Artificial Sequence
  primer
ccagtaacatttgctgaaatatgcgaa

SEQ ID NO: 735 - DNA, 20 bp, Artificial Sequence
  primer
cgtggtgtgcgaccaactaa

SEQ ID NO: 736 - DNA, 26 bp, Artificial Sequence
  primer
ttgtcaactactgcctccacataaaa

SEQ ID NO: 737 - DNA, 23 bp, Artificial Sequence
  primer
aacgtgttgcgattggtgtattg

SEQ ID NO: 738 - DNA, 27 bp, Artificial Sequence
  primer
cattccccatgaacatgctaaactttg

SEQ ID NO: 739 - DNA, 22 bp, Artificial Sequence
  primer
cgtgctttttgctttgctttgt

SEQ ID NO: 740 - DNA, 27 bp, Artificial Sequence
  primer
gaggctgctgttatccacaatagtaat

SEQ ID NO: 741 - DNA, 23 bp, Artificial Sequence
  primer
cctgtgtaggtgttgaggtaggt

SEQ ID NO: 742 - DNA, 25 bp, Artificial Sequence
  primer
tctattatccacacctgcatttgct

SEQ ID NO: 743 - DNA, 20 bp, Artificial Sequence
  primer
ccaggcccattttgtagctt

SEQ ID NO: 744 - DNA, 23 bp, Artificial Sequence
  primer
aggtcaggaaaacagggatttgg

SEQ ID NO: 745 - DNA, 26 bp, Artificial Sequence
  primer
tggagtaaacccaacaatagcagaag

SEQ ID NO: 746 - DNA, 24 bp, Artificial Sequence
  primer
catttgtaacgcaacagggctaat

SEQ ID NO: 747 - DNA, 26 bp, Artificial Sequence
  primer
cgtatgcatgggtattggtatttgtg

SEQ ID NO: 748 - DNA, 28 bp, Artificial Sequence
  primer
catgtatatgcaatagtaacatgggcaa

SEQ ID NO: 749 - DNA, 22 bp, Artificial Sequence
  primer
gaggacgttagggacaatgtgt

SEQ ID NO: 750 - DNA, 23 bp, Artificial Sequence
  primer
ccctgtgataaaggacgcgattt

SEQ ID NO: 751 - DNA, 26 bp, Artificial Sequence
  primer
cgccctagtgagtaacaactgtattt

SEQ ID NO: 752 - DNA, 27 bp, Artificial Sequence
  primer
ggaggattgtaggataaaatggatgct

SEQ ID NO: 753 - DNA, 27 bp, Artificial Sequence
  primer
gtgaaacaccagaatggatagaaagac

SEQ ID NO: 754 - DNA, 25 bp, Artificial Sequence
  primer
tgcacatgcattactatcactgtca

SEQ ID NO: 755 - DNA, 25 bp, Artificial Sequence
  primer
gcattgtgctatgctttttgctttg

SEQ ID NO: 756 - DNA, 25 bp, Artificial Sequence
  primer
acaacgtaatggagaggttgcaata

SEQ ID NO: 757 - DNA, 20 bp, Artificial Sequence
  primer
gcttagtttgggcctgtgtt

SEQ ID NO: 758 - DNA, 27 bp, Artificial Sequence
  primer
accaccggcatatctattagagttttc

SEQ ID NO: 759 - DNA, 25 bp, Artificial Sequence
  primer
tgtgtgtgttgtgtatgttgtcctt

SEQ ID NO: 760 - DNA, 26 bp, Artificial Sequence
  primer
caacttttactatggcgtgacaccta

SEQ ID NO: 761 - DNA, 21 bp, Artificial Sequence
  primer
cggagccaaacatgtgcattg

SEQ ID NO: 762 - DNA, 26 bp, Artificial Sequence
  primer
cgttatcatatgcccactgtaccatt

SEQ ID NO: 763 - DNA, 25 bp, Artificial Sequence
  primer
ccatttctacctatgcttggttgct

SEQ ID NO: 764 - DNA, 25 bp, Artificial Sequence
  primer
gttgtgtcatatgctgtgcatgaaa

SEQ ID NO: 765 - DNA, 27 bp, Artificial Sequence
  primer
catgtgtaggccttgaaataggtagag

SEQ ID NO: 766 - DNA, 22 bp, Artificial Sequence
  primer
cctattatcagcacccggttgt

SEQ ID NO: 767 - DNA, 19 bp, Artificial Sequence
  primer
cttgccctaccctgcattg

SEQ ID NO: 768 - DNA, 27 bp, Artificial Sequence
  primer
cggttaggcatacaaaatggaggaaat

SEQ ID NO: 769 - DNA, 27 bp, Artificial Sequence
  primer
gctatgtatttcagctgcaagtatgct

SEQ ID NO: 770 - DNA, 25 bp, Artificial Sequence
  primer
cattctggtgtttctccatcaacct

SEQ ID NO: 771 - DNA, 27 bp, Artificial Sequence
  primer
cgttcgctattgctatctgtgtcatta

SEQ ID NO: 772 - DNA, 27 bp, Artificial Sequence
  primer
gccaaatattgtgcatgagcgttaatc

SEQ ID NO: 773 - DNA, 27 bp, Artificial Sequence
  primer
ggtacagataacagggaatgcatttct

SEQ ID NO: 774 - DNA, 26 bp, Artificial Sequence
  primer
gacattctcctgcttttacctggtta

SEQ ID NO: 775 - DNA, 24 bp, Artificial Sequence
  primer
aacattcctacctcagcagaacac

SEQ ID NO: 776 - DNA, 23 bp, Artificial Sequence
  primer
tgggtggaccacaagtatgaaaa

SEQ ID NO: 777 - DNA, 27 bp, Artificial Sequence
  primer
agggttactgtaggaaagggattaagt

SEQ ID NO: 778 - DNA, 28 bp, Artificial Sequence
  primer
cgtatcccctgttaccacactaatattg

SEQ ID NO: 779 - DNA, 25 bp, Artificial Sequence
  primer
ttggtgtggtttggtgtgtgtatat

SEQ ID NO: 780 - DNA, 26 bp, Artificial Sequence
  primer
ctccaatggtgtggtacgtataagaa

SEQ ID NO: 781 - DNA, 20 bp, Artificial Sequence
  primer
ccagccattgggtgttggta

SEQ ID NO: 782 - DNA, 26 bp, Artificial Sequence
  primer
gcctataatgcacaactgtgtctgtt

SEQ ID NO: 783 - DNA, 20 bp, Artificial Sequence
  primer
cattttgtggcgaccgaagt

SEQ ID NO: 784 - DNA, 26 bp, Artificial Sequence
  primer
cctggacaggatgatgagtaataagg

SEQ ID NO: 785 - DNA, 25 bp, Artificial Sequence
  primer
gcaacgttatacgcccatatccaat

SEQ ID NO: 786 - DNA, 23 bp, Artificial Sequence
  primer
ggtacgtgcaacaatgtgcttaa

SEQ ID NO: 787 - DNA, 21 bp, Artificial Sequence
  primer
tgcttttgcttggttgttggt

SEQ ID NO: 788 - DNA, 27 bp, Artificial Sequence
  primer
catcacaggtatgttacactgtactgt

SEQ ID NO: 789 - DNA, 25 bp, Artificial Sequence
  primer
ggcatgtgtaggtatggaaattggt

SEQ ID NO: 790 - DNA, 25 bp, Artificial Sequence
  primer
acatcctgcgtaataacagctgtag

SEQ ID NO: 791 - DNA, 23 bp, Artificial Sequence
  primer
atttcggttgcctgtggcttata

SEQ ID NO: 792 - DNA, 25 bp, Artificial Sequence
  primer
cagttgtgcaagccattgttttagt

SEQ ID NO: 793 - DNA, 24 bp, Artificial Sequence
  primer
gatggaggcaactggagagaaatt

SEQ ID NO: 794 - DNA, 26 bp, Artificial Sequence
  primer
gtgtttggtgggccatatatgactat

SEQ ID NO: 795 - DNA, 25 bp, Artificial Sequence
  primer
aagccaatatgtgctgctaattgta

SEQ ID NO: 796 - DNA, 23 bp, Artificial Sequence
  primer
aacacgtattgggacagcagtag

SEQ ID NO: 797 - DNA, 19 bp, Artificial Sequence
  primer
acacccctccacaggctaa

SEQ ID NO: 798 - DNA, 20 bp, Artificial Sequence
  primer
tgtacgccaacctgcaacaa

SEQ ID NO: 799 - DNA, 27 bp, Artificial Sequence
  primer
gggtattacattatccccgtaggtcaa

SEQ ID NO: 800 - DNA, 23 bp, Artificial Sequence
  primer
gctgcagctgtaacaaaatggaa

SEQ ID NO: 801 - DNA, 27 bp, Artificial Sequence
  primer
caccatcagttgcagaaggattaaaag

SEQ ID NO: 802 - DNA, 25 bp, Artificial Sequence
  primer
ctgtgacattagtttggacactgtt

SEQ ID NO: 803 - DNA, 24 bp, Artificial Sequence
  primer
caacacaagccaatattgctgcta

SEQ ID NO: 804 - DNA, 23 bp, Artificial Sequence
  primer
cctgcgcatacaccgatatagat

SEQ ID NO: 805 - DNA, 27 bp, Artificial Sequence
  primer
ggactatatgttttgggaggtggattt

SEQ ID NO: 806 - DNA, 22 bp, Artificial Sequence
  primer
gatgcagggcgttttagtttgg

SEQ ID NO: 807 - DNA, 19 bp, Artificial Sequence
  primer
tcggttggtcttggcacaa

SEQ ID NO: 808 - DNA, 21 bp, Artificial Sequence
  primer
tttaggcgggacaacaagtgt

SEQ ID NO: 809 - DNA, 25 bp, Artificial Sequence
  primer
cagatgatagccaaattgcgtttca

SEQ ID NO: 810 - DNA, 27 bp, Artificial Sequence
  primer
gctgttgtgcccttttataatgtctac

SEQ ID NO: 811 - DNA, 28 bp, Artificial Sequence
  primer
tgctacgcatatatattgcaaccattga

SEQ ID NO: 812 - DNA, 28 bp, Artificial Sequence
  primer
ggatgtggctataacaaaccaaaacaat

SEQ ID NO: 813 - DNA, 21 bp, Artificial Sequence
  primer
tgtactcccgctatgggtgaa

SEQ ID NO: 814 - DNA, 25 bp, Artificial Sequence
  primer
gtgtctatcatgtccccatcctcta

SEQ ID NO: 815 - DNA, 21 bp, Artificial Sequence
  primer
aattcggttgcatggcctagt

SEQ ID NO: 816 - DNA, 26 bp, Artificial Sequence
  primer
gggtgcggtactgtacataattcaag

SEQ ID NO: 817 - DNA, 27 bp, Artificial Sequence
  primer
caatgggacaatggatacaaagtaggt

SEQ ID NO: 818 - DNA, 23 bp, Artificial Sequence
  primer
gggccacacagtaacatacaact

SEQ ID NO: 819 - DNA, 27 bp, Artificial Sequence
  primer
tctatatatgcttggttgctggtgttg

SEQ ID NO: 820 - DNA, 27 bp, Artificial Sequence
  primer
catgtgcagaaccagtatacagttagt

SEQ ID NO: 821 - DNA, 21 bp, Artificial Sequence
  primer
cgtttggtctgggcatgtgta

SEQ ID NO: 822 - DNA, 23 bp, Artificial Sequence
  primer
gctgtgcgggatatctgttactg

SEQ ID NO: 823 - DNA, 27 bp, Artificial Sequence
  primer
tctatgagtaaggtgctgtccctaaat

SEQ ID NO: 824 - DNA, 26 bp, Artificial Sequence
  primer
ggaggtaaagtaaaatggaggcagta

SEQ ID NO: 825 - DNA, 25 bp, Artificial Sequence
  primer
gtgcatgttaattgaaccacccaaa

SEQ ID NO: 826 - DNA, 25 bp, Artificial Sequence
  primer
tcaaacacgctatcatcaactccat

SEQ ID NO: 827 - DNA, 20 bp, Artificial Sequence
  primer
gttgcaatgtcccgcttctg

SEQ ID NO: 828 - DNA, 27 bp, Artificial Sequence
  primer
catgggcatatagtagtaacagtggaa

SEQ ID NO: 829 - DNA, 20 bp, Artificial Sequence
  primer
gctgtgtacctgccattgga

SEQ ID NO: 830 - DNA, 26 bp, Artificial Sequence
  primer
ctgtgtctaccatatcaccatcttca

SEQ ID NO: 831 - DNA, 23 bp, Artificial Sequence
  primer
ggttgcacccaatgagtaaggta

SEQ ID NO: 832 - DNA, 25 bp, Artificial Sequence
  primer
gcaaaactggacattcaggacaaaa

SEQ ID NO: 833 - DNA, 25 bp, Artificial Sequence
  primer
agacatagatagcaatgcacaagca

SEQ ID NO: 834 - DNA, 27 bp, Artificial Sequence
  primer
atcacccccttcatctactttactaca

SEQ ID NO: 835 - DNA, 22 bp, Artificial Sequence
  primer
gtttgtctgtgtgtgtgccatt

SEQ ID NO: 836 - DNA, 27 bp, Artificial Sequence
  primer
gcatggcaatatatacacagtgtaggt

SEQ ID NO: 837 - DNA, 21 bp, Artificial Sequence
  primer
gtaggccgaggtcaaccttta

SEQ ID NO: 838 - DNA, 25 bp, Artificial Sequence
  primer
gtgcacatcccacaatacataactg

SEQ ID NO: 839 - DNA, 26 bp, Artificial Sequence
  primer
ggttaggtggtgttccttactgttta

SEQ ID NO: 840 - DNA, 22 bp, Artificial Sequence
  primer
caaaaggctaggcaaccgaatt

SEQ ID NO: 841 - DNA, 20 bp, Artificial Sequence
  primer
cgacacgccggaatggataa

SEQ ID NO: 842 - DNA, 26 bp, Artificial Sequence
  primer
cgctgcagcattactattacaatctg

SEQ ID NO: 843 - DNA, 23 bp, Artificial Sequence
  primer
ggtgtggttttgtgtatgcatgt

SEQ ID NO: 844 - DNA, 26 bp, Artificial Sequence
  primer
ggtatacagcaaacacctcaaatggt

SEQ ID NO: 845 - DNA, 25 bp, Artificial Sequence
  primer
gcctgtgttggtgttgaaataggta

SEQ ID NO: 846 - DNA, 26 bp, Artificial Sequence
  primer
tgcaacattgtccctactgtctttag

SEQ ID NO: 847 - DNA, 25 bp, Artificial Sequence
  primer
ccctgtgactaacatatgtccttgt

SEQ ID NO: 848 - DNA, 24 bp, Artificial Sequence
  primer
ccacacggtatagtttgcaaccat

SEQ ID NO: 849 - DNA, 25 bp, Artificial Sequence
  primer
gaacgcatgttaattgaacctccaa

SEQ ID NO: 850 - DNA, 27 bp, Artificial Sequence
  primer
gctgcactaacgtttgtcttttaatcc

SEQ ID NO: 851 - DNA, 25 bp, Artificial Sequence
  primer
tcgcttgcagtgtctgtgtatattt

SEQ ID NO: 852 - DNA, 26 bp, Artificial Sequence
  primer
catggtaatgtacaagtgccatagga

SEQ ID NO: 853 - DNA, 27 bp, Artificial Sequence
  primer
tgtattttaggttgtaggcctccctta

SEQ ID NO: 854 - DNA, 27 bp, Artificial Sequence
  primer
ctccaaagccaacatctatcatatcac

SEQ ID NO: 855 - DNA, 25 bp, Artificial Sequence
  primer
gtcgccattttacatgcattaaggt

SEQ ID NO: 856 - DNA, 22 bp, Artificial Sequence
  primer
aggaaacaaaccctgccaagtt

SEQ ID NO: 857 - DNA, 21 bp, Artificial Sequence
  primer
cgtagtacagccgttgcattg

SEQ ID NO: 858 - DNA, 24 bp, Artificial Sequence
  primer
cccattgtaccatttgcgatagtt

SEQ ID NO: 859 - DNA, 27 bp, Artificial Sequence
  primer
gctgctaagtgtatatagttactcgca

SEQ ID NO: 860 - DNA, 24 bp, Artificial Sequence
  primer
ctgctgcaaacacatattgggatt

SEQ ID NO: 861 - DNA, 25 bp, Artificial Sequence
  primer
ggatgtgttggtgttgaagtaggta

SEQ ID NO: 862 - DNA, 20 bp, Artificial Sequence
  primer
tcctgttggtcgttgccatt

SEQ ID NO: 863 - DNA, 22 bp, Artificial Sequence
  primer
cctgtaggttaagggtggtgtt

SEQ ID NO: 864 - DNA, 21 bp, Artificial Sequence
  primer
aaatcggtcgccacaaaatgg

SEQ ID NO: 865 - DNA, 27 bp, Artificial Sequence
  primer
agtacagacctacgtgaccatatagac

SEQ ID NO: 866 - DNA, 19 bp, Artificial Sequence
  primer
ctgagaagccctgcccttc

SEQ ID NO: 867 - DNA, 27 bp, Artificial Sequence
  primer
agtacagacctacgtgaccatatagac

SEQ ID NO: 868 - DNA, 19 bp, Artificial Sequence
  primer
aaatacggctgcaccgagt

SEQ ID NO: 869 - DNA, 27 bp, Artificial Sequence
  primer
agtacagacctacgtgaccatatagac

SEQ ID NO: 870 - DNA, 24 bp, Artificial Sequence
  primer
ggtgatccagactctgaccttttg

SEQ ID NO: 871 - DNA, 27 bp, Artificial Sequence
  primer
agtacagacctacgtgaccatatagac

SEQ ID NO: 872 - DNA, 22 bp, Artificial Sequence
  primer
atcatgatggctgtatgtgcca

SEQ ID NO: 873 - DNA, 27 bp, Artificial Sequence
  primer
agtacagacctacgtgaccatatagac

SEQ ID NO: 874 - DNA, 21 bp, Artificial Sequence
  primer
catggttccaccagcgttatt

SEQ ID NO: 875 - DNA, 27 bp, Artificial Sequence
  primer
agtacagacctacgtgaccatatagac

SEQ ID NO: 876 - DNA, 18 bp, Artificial Sequence
  primer
tctttgctcgcagctcgt

SEQ ID NO: 877 - DNA, 23 bp, Artificial Sequence
  primer
gctcacacaaaggacggattaac

SEQ ID NO: 878 - DNA, 19 bp, Artificial Sequence
  primer
ctgagaagccctgcccttc

SEQ ID NO: 879 - DNA, 23 bp, Artificial Sequence
  primer
gctcacacaaaggacggattaac

SEQ ID NO: 880 - DNA, 19 bp, Artificial Sequence
  primer
aaatacggctgcaccgagt

SEQ ID NO: 881 - DNA, 23 bp, Artificial Sequence
  primer
gctcacacaaaggacggattaac

SEQ ID NO: 882 - DNA, 24 bp, Artificial Sequence
  primer
ggtgatccagactctgaccttttg

SEQ ID NO: 883 - DNA, 23 bp, Artificial Sequence
  primer
gctcacacaaaggacggattaac

SEQ ID NO: 884 - DNA, 22 bp, Artificial Sequence
  primer
atcatgatggctgtatgtgcca

SEQ ID NO: 885 - DNA, 23 bp, Artificial Sequence
  primer
gctcacacaaaggacggattaac

SEQ ID NO: 886 - DNA, 21 bp, Artificial Sequence
  primer
catggttccaccagcgttatt

SEQ ID NO: 887 - DNA, 23 bp, Artificial Sequence
  primer
gctcacacaaaggacggattaac

SEQ ID NO: 888 - DNA, 18 bp, Artificial Sequence
  primer
tctttgctcgcagctcgt

SEQ ID NO: 889 - DNA, 21 bp, Artificial Sequence
  primer
ggaattgtgtgccccatctgt

SEQ ID NO: 890 - DNA, 19 bp, Artificial Sequence
  primer
ctgagaagccctgcccttc

SEQ ID NO: 891 - DNA, 21 bp, Artificial Sequence
  primer
ggaattgtgtgccccatctgt

SEQ ID NO: 892 - DNA, 19 bp, Artificial Sequence
  primer
aaatacggctgcaccgagt

SEQ ID NO: 893 - DNA, 21 bp, Artificial Sequence
  primer
ggaattgtgtgccccatctgt

SEQ ID NO: 894 - DNA, 24 bp, Artificial Sequence
  primer
ggtgatccagactctgaccttttg

SEQ ID NO: 895 - DNA, 21 bp, Artificial Sequence
  primer
ggaattgtgtgccccatctgt

SEQ ID NO: 896 - DNA, 22 bp, Artificial Sequence
  primer
atcatgatggctgtatgtgcca

SEQ ID NO: 897 - DNA, 21 bp, Artificial Sequence
  primer
ggaattgtgtgccccatctgt

SEQ ID NO: 898 - DNA, 21 bp, Artificial Sequence
  primer
catggttccaccagcgttatt

SEQ ID NO: 899 - DNA, 21 bp, Artificial Sequence
  primer
ggaattgtgtgccccatctgt

SEQ ID NO: 900 - DNA, 18 bp, Artificial Sequence
  primer
tctttgctcgcagctcgt

SEQ ID NO: 901 - DNA, 28 bp, Artificial Sequence
  primer
aatgacagtaaagacatagacagccaaa

SEQ ID NO: 902 - DNA, 19 bp, Artificial Sequence
  primer
ctgagaagccctgcccttc

SEQ ID NO: 903 - DNA, 28 bp, Artificial Sequence
  primer
aatgacagtaaagacatagacagccaaa

SEQ ID NO: 904 - DNA, 19 bp, Artificial Sequence
  primer
aaatacggctgcaccgagt

SEQ ID NO: 905 - DNA, 28 bp, Artificial Sequence
  primer
aatgacagtaaagacatagacagccaaa

SEQ ID NO: 906 - DNA, 24 bp, Artificial Sequence
  primer
ggtgatccagactctgaccttttg

SEQ ID NO: 907 - DNA, 28 bp, Artificial Sequence
  primer
aatgacagtaaagacatagacagccaaa

SEQ ID NO: 908 - DNA, 22 bp, Artificial Sequence
  primer
atcatgatggctgtatgtgcca

SEQ ID NO: 909 - DNA, 28 bp, Artificial Sequence
  primer
aatgacagtaaagacatagacagccaaa

SEQ ID NO: 910 - DNA, 21 bp, Artificial Sequence
  primer
catggttccaccagcgttatt

SEQ ID NO: 911 - DNA, 28 bp, Artificial Sequence
  primer
aatgacagtaaagacatagacagccaaa

SEQ ID NO: 912 - DNA, 18 bp, Artificial Sequence
  primer
tctttgctcgcagctcgt

SEQ ID NO: 913 - DNA, 22 bp, Artificial Sequence
  primer
cagctacacctacaggcaacaa

SEQ ID NO: 914 - DNA, 19 bp, Artificial Sequence
  primer
ctgagaagccctgcccttc

SEQ ID NO: 915 - DNA, 22 bp, Artificial Sequence
  primer
cagctacacctacaggcaacaa

SEQ ID NO: 916 - DNA, 19 bp, Artificial Sequence
  primer
aaatacggctgcaccgagt

SEQ ID NO: 917 - DNA, 22 bp, Artificial Sequence
  primer
cagctacacctacaggcaacaa

SEQ ID NO: 918 - DNA, 24 bp, Artificial Sequence
  primer
ggtgatccagactctgaccttttg

SEQ ID NO: 919 - DNA, 22 bp, Artificial Sequence
  primer
cagctacacctacaggcaacaa

SEQ ID NO: 920 - DNA, 22 bp, Artificial Sequence
  primer
atcatgatggctgtatgtgcca

SEQ ID NO: 921 - DNA, 22 bp, Artificial Sequence
  primer
cagctacacctacaggcaacaa

SEQ ID NO: 922 - DNA, 21 bp, Artificial Sequence
  primer
catggttccaccagcgttatt

SEQ ID NO: 923 - DNA, 22 bp, Artificial Sequence
  primer
cagctacacctacaggcaacaa

SEQ ID NO: 924 - DNA, 18 bp, Artificial Sequence
  primer
tctttgctcgcagctcgt

SEQ ID NO: 925 - DNA, 20 bp, Artificial Sequence
  primer
tgcatcccagcagtaagcaa

SEQ ID NO: 926 - DNA, 19 bp, Artificial Sequence
  primer
ctgagaagccctgcccttc

SEQ ID NO: 927 - DNA, 20 bp, Artificial Sequence
  primer
tgcatcccagcagtaagcaa

SEQ ID NO: 928 - DNA, 19 bp, Artificial Sequence
  primer
aaatacggctgcaccgagt

SEQ ID NO: 929 - DNA, 20 bp, Artificial Sequence
  primer
tgcatcccagcagtaagcaa

SEQ ID NO: 930 - DNA, 24 bp, Artificial Sequence
  primer
ggtgatccagactctgaccttttg

SEQ ID NO: 931 - DNA, 20 bp, Artificial Sequence
  primer
tgcatcccagcagtaagcaa

SEQ ID NO: 932 - DNA, 22 bp, Artificial Sequence
  primer
atcatgatggctgtatgtgcca

SEQ ID NO: 933 - DNA, 20 bp, Artificial Sequence
  primer
tgcatcccagcagtaagcaa

SEQ ID NO: 934 - DNA, 21 bp, Artificial Sequence
  primer
catggttccaccagcgttatt

SEQ ID NO: 935 - DNA, 20 bp, Artificial Sequence
  primer
tgcatcccagcagtaagcaa

SEQ ID NO: 936 - DNA, 18 bp, Artificial Sequence
  primer
tctttgctcgcagctcgt

SEQ ID NO: 937 - DNA, 27 bp, Artificial Sequence
  primer
caacgtttaaatgtgtgtcaggacaaa

SEQ ID NO: 938 - DNA, 19 bp, Artificial Sequence
  primer
ctgagaagccctgcccttc

SEQ ID NO: 939 - DNA, 27 bp, Artificial Sequence
  primer
caacgtttaaatgtgtgtcaggacaaa

SEQ ID NO: 940 - DNA, 19 bp, Artificial Sequence
  primer
aaatacggctgcaccgagt

SEQ ID NO: 941 - DNA, 27 bp, Artificial Sequence
  primer
caacgtttaaatgtgtgtcaggacaaa

SEQ ID NO: 942 - DNA, 24 bp, Artificial Sequence
  primer
ggtgatccagactctgaccttttg

SEQ ID NO: 943 - DNA, 27 bp, Artificial Sequence
  primer
caacgtttaaatgtgtgtcaggacaaa

SEQ ID NO: 944 - DNA, 22 bp, Artificial Sequence
  primer
atcatgatggctgtatgtgcca

SEQ ID NO: 945 - DNA, 27 bp, Artificial Sequence
  primer
caacgtttaaatgtgtgtcaggacaaa

SEQ ID NO: 946 - DNA, 21 bp, Artificial Sequence
  primer
catggttccaccagcgttatt

SEQ ID NO: 947 - DNA, 27 bp, Artificial Sequence
  primer
caacgtttaaatgtgtgtcaggacaaa

SEQ ID NO: 948 - DNA, 18 bp, Artificial Sequence
  primer
tctttgctcgcagctcgt

SEQ ID NO: 949 - DNA, 19 bp, Artificial Sequence
  primer
cagctgcatgcacaaacca

SEQ ID NO: 950 - DNA, 19 bp, Artificial Sequence
  primer
ctgagaagccctgcccttc

SEQ ID NO: 951 - DNA, 19 bp, Artificial Sequence
  primer
cagctgcatgcacaaacca

SEQ ID NO: 952 - DNA, 19 bp, Artificial Sequence
  primer
aaatacggctgcaccgagt

SEQ ID NO: 953 - DNA, 19 bp, Artificial Sequence
  primer
cagctgcatgcacaaacca

SEQ ID NO: 954 - DNA, 24 bp, Artificial Sequence
  primer
ggtgatccagactctgaccttttg

SEQ ID NO: 955 - DNA, 19 bp, Artificial Sequence
  primer
cagctgcatgcacaaacca

SEQ ID NO: 956 - DNA, 22 bp, Artificial Sequence
  primer
atcatgatggctgtatgtgcca

SEQ ID NO: 957 - DNA, 19 bp, Artificial Sequence
  primer
cagctgcatgcacaaacca

SEQ ID NO: 958 - DNA, 21 bp, Artificial Sequence
  primer
catggttccaccagcgttatt

SEQ ID NO: 959 - DNA, 19 bp, Artificial Sequence
  primer
cagctgcatgcacaaacca

SEQ ID NO: 960 - DNA, 18 bp, Artificial Sequence
  primer
tctttgctcgcagctcgt

SEQ ID NO: 961 - DNA, 19 bp, Artificial Sequence
  primer
aatcgtgtgccccaactgt

SEQ ID NO: 962 - DNA, 19 bp, Artificial Sequence
  primer
ctgagaagccctgcccttc

SEQ ID NO: 963 - DNA, 19 bp, Artificial Sequence
  primer
aatcgtgtgccccaactgt

SEQ ID NO: 964 - DNA, 19 bp, Artificial Sequence
  primer
aaatacggctgcaccgagt

SEQ ID NO: 965 - DNA, 19 bp, Artificial Sequence
  primer
aatcgtgtgccccaactgt

SEQ ID NO: 966 - DNA, 24 bp, Artificial Sequence
  primer
ggtgatccagactctgaccttttg

SEQ ID NO: 967 - DNA, 19 bp, Artificial Sequence
  primer
aatcgtgtgccccaactgt

SEQ ID NO: 968 - DNA, 22 bp, Artificial Sequence
  primer
atcatgatggctgtatgtgcca

SEQ ID NO: 969 - DNA, 19 bp, Artificial Sequence
  primer
aatcgtgtgccccaactgt

SEQ ID NO: 970 - DNA, 21 bp, Artificial Sequence
  primer
catggttccaccagcgttatt

SEQ ID NO: 971 - DNA, 19 bp, Artificial Sequence
  primer
aatcgtgtgccccaactgt

SEQ ID NO: 972 - DNA, 18 bp, Artificial Sequence
  primer
tctttgctcgcagctcgt

SEQ ID NO: 973 - DNA, 29 bp, Artificial Sequence
  primer
gtgcaggagaaaatactagatctttacga

SEQ ID NO: 974 - DNA, 19 bp, Artificial Sequence
  primer
ctgagaagccctgcccttc

SEQ ID NO: 975 - DNA, 29 bp, Artificial Sequence
  primer
gtgcaggagaaaatactagatctttacga

SEQ ID NO: 976 - DNA, 19 bp, Artificial Sequence
  primer
aaatacggctgcaccgagt

SEQ ID NO: 977 - DNA, 29 bp, Artificial Sequence
  primer
gtgcaggagaaaatactagatctttacga

SEQ ID NO: 978 - DNA, 24 bp, Artificial Sequence
  primer
ggtgatccagactctgaccttttg

SEQ ID NO: 979 - DNA, 29 bp, Artificial Sequence
  primer
gtgcaggagaaaatactagatctttacga

SEQ ID NO: 980 - DNA, 22 bp, Artificial Sequence
  primer
atcatgatggctgtatgtgcca

SEQ ID NO: 981 - DNA, 29 bp, Artificial Sequence
  primer
gtgcaggagaaaatactagatctttacga

SEQ ID NO: 982 - DNA, 21 bp, Artificial Sequence
  primer
catggttccaccagcgttatt

SEQ ID NO: 983 - DNA, 29 bp, Artificial Sequence
  primer
gtgcaggagaaaatactagatctttacga

SEQ ID NO: 984 - DNA, 18 bp, Artificial Sequence
  primer
tctttgctcgcagctcgt

SEQ ID NO: 985 - DNA, 23 bp, Artificial Sequence
  primer
acgtactgcaactaactgcacaa

SEQ ID NO: 986 - DNA, 19 bp, Artificial Sequence
  primer
ctgagaagccctgcccttc

SEQ ID NO: 987 - DNA, 23 bp, Artificial Sequence
  primer
acgtactgcaactaactgcacaa

SEQ ID NO: 988 - DNA, 19 bp, Artificial Sequence
  primer
aaatacggctgcaccgagt

SEQ ID NO: 989 - DNA, 23 bp, Artificial Sequence
  primer
acgtactgcaactaactgcacaa

SEQ ID NO: 990 - DNA, 24 bp, Artificial Sequence
  primer
ggtgatccagactctgaccttttg

SEQ ID NO: 991 - DNA, 23 bp, Artificial Sequence
  primer
acgtactgcaactaactgcacaa

SEQ ID NO: 992 - DNA, 22 bp, Artificial Sequence
  primer
atcatgatggctgtatgtgcca

SEQ ID NO: 993 - DNA, 23 bp, Artificial Sequence
  primer
acgtactgcaactaactgcacaa

SEQ ID NO: 994 - DNA, 21 bp, Artificial Sequence
  primer
catggttccaccagcgttatt

SEQ ID NO: 995 - DNA, 23 bp, Artificial Sequence
  primer
acgtactgcaactaactgcacaa

SEQ ID NO: 996 - DNA, 18 bp, Artificial Sequence
  primer
tctttgctcgcagctcgt

SEQ ID NO: 997 - DNA, 19 bp, Artificial Sequence
  primer
gtgccctacctgtgcacaa

SEQ ID NO: 998 - DNA, 19 bp, Artificial Sequence
  primer
ctgagaagccctgcccttc

SEQ ID NO: 999 - DNA, 19 bp, Artificial Sequence
  primer
gtgccctacctgtgcacaa

SEQ ID NO: 1000 - DNA, 19 bp, Artificial Sequence
  primer
aaatacggctgcaccgagt

SEQ ID NO: 1001 - DNA, 19 bp, Artificial Sequence
  primer
gtgccctacctgtgcacaa

SEQ ID NO: 1002 - DNA, 24 bp, Artificial Sequence
  primer
ggtgatccagactctgaccttttg

SEQ ID NO: 1003 - DNA, 19 bp, Artificial Sequence
  primer
gtgccctacctgtgcacaa

SEQ ID NO: 1004 - DNA, 22 bp, Artificial Sequence
  primer
atcatgatggctgtatgtgcca

SEQ ID NO: 1005 - DNA, 19 bp, Artificial Sequence
  primer
gtgccctacctgtgcacaa

SEQ ID NO: 1006 - DNA, 21 bp, Artificial Sequence
  primer
catggttccaccagcgttatt

SEQ ID NO: 1007 - DNA, 19 bp, Artificial Sequence
  primer
gtgccctacctgtgcacaa

SEQ ID NO: 1008 - DNA, 18 bp, Artificial Sequence
  primer
tctttgctcgcagctcgt

SEQ ID NO: 1009 - DNA, 27 bp, Artificial Sequence
  primer
attacgagactgatagcacatgtttgt

SEQ ID NO: 1010 - DNA, 19 bp, Artificial Sequence
  primer
ctgagaagccctgcccttc

SEQ ID NO: 1011 - DNA, 27 bp, Artificial Sequence
  primer
attacgagactgatagcacatgtttgt

SEQ ID NO: 1012 - DNA, 19 bp, Artificial Sequence
  primer
aaatacggctgcaccgagt

SEQ ID NO: 1013 - DNA, 27 bp, Artificial Sequence
  primer
attacgagactgatagcacatgtttgt

SEQ ID NO: 1014 - DNA, 24 bp, Artificial Sequence
  primer
ggtgatccagactctgaccttttg

SEQ ID NO: 1015 - DNA, 27 bp, Artificial Sequence
  primer
attacgagactgatagcacatgtttgt

SEQ ID NO: 1016 - DNA, 22 bp, Artificial Sequence
  primer
atcatgatggctgtatgtgcca

SEQ ID NO: 1017 - DNA, 27 bp, Artificial Sequence
  primer
attacgagactgatagcacatgtttgt

SEQ ID NO: 1018 - DNA, 21 bp, Artificial Sequence
  primer
catggttccaccagcgttatt

SEQ ID NO: 1019 - DNA, 27 bp, Artificial Sequence
  primer
attacgagactgatagcacatgtttgt

SEQ ID NO: 1020 - DNA, 18 bp, Artificial Sequence
  primer
tctttgctcgcagctcgt

SEQ ID NO: 1021 - DNA, 26 bp, Artificial Sequence
  primer
tctacatctgactgcacaaacaaaga

SEQ ID NO: 1022 - DNA, 19 bp, Artificial Sequence
  primer
ctgagaagccctgcccttc

SEQ ID NO: 1023 - DNA, 26 bp, Artificial Sequence
  primer
tctacatctgactgcacaaacaaaga

SEQ ID NO: 1024 - DNA, 19 bp, Artificial Sequence
  primer
aaatacggctgcaccgagt

SEQ ID NO: 1025 - DNA, 26 bp, Artificial Sequence
  primer
tctacatctgactgcacaaacaaaga

SEQ ID NO: 1026 - DNA, 24 bp, Artificial Sequence
  primer
ggtgatccagactctgaccttttg

SEQ ID NO: 1027 - DNA, 26 bp, Artificial Sequence
  primer
tctacatctgactgcacaaacaaaga

SEQ ID NO: 1028 - DNA, 22 bp, Artificial Sequence
  primer
atcatgatggctgtatgtgcca

SEQ ID NO: 1029 - DNA, 26 bp, Artificial Sequence
  primer
tctacatctgactgcacaaacaaaga

SEQ ID NO: 1030 - DNA, 21 bp, Artificial Sequence
  primer
catggttccaccagcgttatt

SEQ ID NO: 1031 - DNA, 26 bp, Artificial Sequence
  primer
tctacatctgactgcacaaacaaaga

SEQ ID NO: 1032 - DNA, 18 bp, Artificial Sequence
  primer
tctttgctcgcagctcgt

SEQ ID NO: 1033 - DNA, 23 bp, Artificial Sequence
  primer
cggctgttcacagagagcataat

SEQ ID NO: 1034 - DNA, 19 bp, Artificial Sequence
  primer
ctgagaagccctgcccttc

SEQ ID NO: 1035 - DNA, 23 bp, Artificial Sequence
  primer
cggctgttcacagagagcataat

SEQ ID NO: 1036 - DNA, 19 bp, Artificial Sequence
  primer
aaatacggctgcaccgagt

SEQ ID NO: 1037 - DNA, 23 bp, Artificial Sequence
  primer
cggctgttcacagagagcataat

SEQ ID NO: 1038 - DNA, 24 bp, Artificial Sequence
  primer
ggtgatccagactctgaccttttg

SEQ ID NO: 1039 - DNA, 23 bp, Artificial Sequence
  primer
cggctgttcacagagagcataat

SEQ ID NO: 1040 - DNA, 22 bp, Artificial Sequence
  primer
atcatgatggctgtatgtgcca

SEQ ID NO: 1041 - DNA, 23 bp, Artificial Sequence
  primer
cggctgttcacagagagcataat

SEQ ID NO: 1042 - DNA, 21 bp, Artificial Sequence
  primer
catggttccaccagcgttatt

SEQ ID NO: 1043 - DNA, 23 bp, Artificial Sequence
  primer
cggctgttcacagagagcataat

SEQ ID NO: 1044 - DNA, 18 bp, Artificial Sequence
  primer
tctttgctcgcagctcgt

SEQ ID NO: 1045 - DNA, 26 bp, Artificial Sequence
  primer
acaacgtttaaatgtgttacaggaca

SEQ ID NO: 1046 - DNA, 19 bp, Artificial Sequence
  primer
ctgagaagccctgcccttc

SEQ ID NO: 1047 - DNA, 26 bp, Artificial Sequence
  primer
acaacgtttaaatgtgttacaggaca

SEQ ID NO: 1048 - DNA, 19 bp, Artificial Sequence
  primer
aaatacggctgcaccgagt

SEQ ID NO: 1049 - DNA, 26 bp, Artificial Sequence
  primer
acaacgtttaaatgtgttacaggaca

SEQ ID NO: 1050 - DNA, 24 bp, Artificial Sequence
  primer
ggtgatccagactctgaccttttg

SEQ ID NO: 1051 - DNA, 26 bp, Artificial Sequence
  primer
acaacgtttaaatgtgttacaggaca

SEQ ID NO: 1052 - DNA, 22 bp, Artificial Sequence
  primer
atcatgatggctgtatgtgcca

SEQ ID NO: 1053 - DNA, 26 bp, Artificial Sequence
  primer
acaacgtttaaatgtgttacaggaca

SEQ ID NO: 1054 - DNA, 21 bp, Artificial Sequence
  primer
catggttccaccagcgttatt

SEQ ID NO: 1055 - DNA, 26 bp, Artificial Sequence
  primer
acaacgtttaaatgtgttacaggaca

SEQ ID NO: 1056 - DNA, 18 bp, Artificial Sequence
  primer
tctttgctcgcagctcgt

SEQ ID NO: 1057 - DNA, 22 bp, Artificial Sequence
  primer
cacagtaacagtacaggccaca

SEQ ID NO: 1058 - DNA, 19 bp, Artificial Sequence
  primer
ctgagaagccctgcccttc

SEQ ID NO: 1059 - DNA, 22 bp, Artificial Sequence
  primer
cacagtaacagtacaggccaca

SEQ ID NO: 1060 - DNA, 19 bp, Artificial Sequence
  primer
aaatacggctgcaccgagt

SEQ ID NO: 1061 - DNA, 22 bp, Artificial Sequence
  primer
cacagtaacagtacaggccaca

SEQ ID NO: 1062 - DNA, 24 bp, Artificial Sequence
  primer
ggtgatccagactctgaccttttg

SEQ ID NO: 1063 - DNA, 22 bp, Artificial Sequence
  primer
cacagtaacagtacaggccaca

SEQ ID NO: 1064 - DNA, 22 bp, Artificial Sequence
  primer
atcatgatggctgtatgtgcca

SEQ ID NO: 1065 - DNA, 22 bp, Artificial Sequence
  primer
cacagtaacagtacaggccaca

SEQ ID NO: 1066 - DNA, 21 bp, Artificial Sequence
  primer
catggttccaccagcgttatt

SEQ ID NO: 1067 - DNA, 22 bp, Artificial Sequence
  primer
cacagtaacagtacaggccaca

SEQ ID NO: 1068 - DNA, 18 bp, Artificial Sequence
  primer
tctttgctcgcagctcgt

SEQ ID NO: 1069 - DNA, 18 bp, Artificial Sequence
  primer
cgtggtgtgcaactgcaa

SEQ ID NO: 1070 - DNA, 19 bp, Artificial Sequence
  primer
ctgagaagccctgcccttc

SEQ ID NO: 1071 - DNA, 18 bp, Artificial Sequence
  primer
cgtggtgtgcaactgcaa

SEQ ID NO: 1072 - DNA, 19 bp, Artificial Sequence
  primer
aaatacggctgcaccgagt

SEQ ID NO: 1073 - DNA, 18 bp, Artificial Sequence
  primer
cgtggtgtgcaactgcaa

SEQ ID NO: 1074 - DNA, 24 bp, Artificial Sequence
  primer
ggtgatccagactctgaccttttg

SEQ ID NO: 1075 - DNA, 18 bp, Artificial Sequence
  primer
cgtggtgtgcaactgcaa

SEQ ID NO: 1076 - DNA, 22 bp, Artificial Sequence
  primer
atcatgatggctgtatgtgcca

SEQ ID NO: 1077 - DNA, 18 bp, Artificial Sequence
  primer
cgtggtgtgcaactgcaa

SEQ ID NO: 1078 - DNA, 21 bp, Artificial Sequence
  primer
catggttccaccagcgttatt

SEQ ID NO: 1079 - DNA, 18 bp, Artificial Sequence
  primer
cgtggtgtgcaactgcaa

SEQ ID NO: 1080 - DNA, 18 bp, Artificial Sequence
  primer
tctttgctcgcagctcgt

SEQ ID NO: 1081 - DNA, 28 bp, Artificial Sequence
  primer
cgttacaggacaaaatactagaccacta

SEQ ID NO: 1082 - DNA, 19 bp, Artificial Sequence
  primer
ctgagaagccctgcccttc

SEQ ID NO: 1083 - DNA, 28 bp, Artificial Sequence
  primer
cgttacaggacaaaatactagaccacta

SEQ ID NO: 1084 - DNA, 19 bp, Artificial Sequence
  primer
aaatacggctgcaccgagt

SEQ ID NO: 1085 - DNA, 28 bp, Artificial Sequence
  primer
cgttacaggacaaaatactagaccacta

SEQ ID NO: 1086 - DNA, 24 bp, Artificial Sequence
  primer
ggtgatccagactctgaccttttg

SEQ ID NO: 1087 - DNA, 28 bp, Artificial Sequence
  primer
cgttacaggacaaaatactagaccacta

SEQ ID NO: 1088 - DNA, 22 bp, Artificial Sequence
  primer
atcatgatggctgtatgtgcca

SEQ ID NO: 1089 - DNA, 28 bp, Artificial Sequence
  primer
cgttacaggacaaaatactagaccacta

SEQ ID NO: 1090 - DNA, 21 bp, Artificial Sequence
  primer
catggttccaccagcgttatt

SEQ ID NO: 1091 - DNA, 28 bp, Artificial Sequence
  primer
cgttacaggacaaaatactagaccacta

SEQ ID NO: 1092 - DNA, 18 bp, Artificial Sequence
  primer
tctttgctcgcagctcgt

SEQ ID NO: 1093 - DNA, 28 bp, Artificial Sequence
  primer
tcctgtgttcaagtacaagtaacaacaa

SEQ ID NO: 1094 - DNA, 19 bp, Artificial Sequence
  primer
ctgagaagccctgcccttc

SEQ ID NO: 1095 - DNA, 28 bp, Artificial Sequence
  primer
tcctgtgttcaagtacaagtaacaacaa

SEQ ID NO: 1096 - DNA, 19 bp, Artificial Sequence
  primer
aaatacggctgcaccgagt

SEQ ID NO: 1097 - DNA, 28 bp, Artificial Sequence
  primer
tcctgtgttcaagtacaagtaacaacaa

SEQ ID NO: 1098 - DNA, 24 bp, Artificial Sequence
  primer
ggtgatccagactctgaccttttg

SEQ ID NO: 1099 - DNA, 28 bp, Artificial Sequence
  primer
tcctgtgttcaagtacaagtaacaacaa

SEQ ID NO: 1100 - DNA, 22 bp, Artificial Sequence
  primer
atcatgatggctgtatgtgcca

SEQ ID NO: 1101 - DNA, 28 bp, Artificial Sequence
  primer
tcctgtgttcaagtacaagtaacaacaa

SEQ ID NO: 1102 - DNA, 21 bp, Artificial Sequence
  primer
catggttccaccagcgttatt

SEQ ID NO: 1103 - DNA, 28 bp, Artificial Sequence
  primer
tcctgtgttcaagtacaagtaacaacaa

SEQ ID NO: 1104 - DNA, 18 bp, Artificial Sequence
  primer
tctttgctcgcagctcgt

SEQ ID NO: 1105 - DNA, 21 bp, Artificial Sequence
  primer
agcaccttgtcctttgtgtgt

SEQ ID NO: 1106 - DNA, 19 bp, Artificial Sequence
  primer
ctgagaagccctgcccttc

SEQ ID NO: 1107 - DNA, 21 bp, Artificial Sequence
  primer
agcaccttgtcctttgtgtgt

SEQ ID NO: 1108 - DNA, 19 bp, Artificial Sequence
  primer
aaatacggctgcaccgagt

SEQ ID NO: 1109 - DNA, 21 bp, Artificial Sequence
  primer
agcaccttgtcctttgtgtgt

SEQ ID NO: 1110 - DNA, 24 bp, Artificial Sequence
  primer
ggtgatccagactctgaccttttg

SEQ ID NO: 1111 - DNA, 21 bp, Artificial Sequence
  primer
agcaccttgtcctttgtgtgt

SEQ ID NO: 1112 - DNA, 22 bp, Artificial Sequence
  primer
atcatgatggctgtatgtgcca

SEQ ID NO: 1113 - DNA, 21 bp, Artificial Sequence
  primer
agcaccttgtcctttgtgtgt

SEQ ID NO: 1114 - DNA, 21 bp, Artificial Sequence
  primer
catggttccaccagcgttatt

SEQ ID NO: 1115 - DNA, 21 bp, Artificial Sequence
  primer
agcaccttgtcctttgtgtgt

SEQ ID NO: 1116 - DNA, 18 bp, Artificial Sequence
  primer
tctttgctcgcagctcgt

SEQ ID NO: 1117 - DNA, 28 bp, Artificial Sequence
  primer
gtgccaggagaaaatactagactgttat

SEQ ID NO: 1118 - DNA, 19 bp, Artificial Sequence
  primer
ctgagaagccctgcccttc

SEQ ID NO: 1119 - DNA, 28 bp, Artificial Sequence
  primer
gtgccaggagaaaatactagactgttat

SEQ ID NO: 1120 - DNA, 19 bp, Artificial Sequence
  primer
aaatacggctgcaccgagt

SEQ ID NO: 1121 - DNA, 28 bp, Artificial Sequence
  primer
gtgccaggagaaaatactagactgttat

SEQ ID NO: 1122 - DNA, 24 bp, Artificial Sequence
  primer
ggtgatccagactctgaccttttg

SEQ ID NO: 1123 - DNA, 28 bp, Artificial Sequence
  primer
gtgccaggagaaaatactagactgttat

SEQ ID NO: 1124 - DNA, 22 bp, Artificial Sequence
  primer
atcatgatggctgtatgtgcca

SEQ ID NO: 1125 - DNA, 28 bp, Artificial Sequence
  primer
gtgccaggagaaaatactagactgttat

SEQ ID NO: 1126 - DNA, 21 bp, Artificial Sequence
  primer
catggttccaccagcgttatt

SEQ ID NO: 1127 - DNA, 28 bp, Artificial Sequence
  primer
gtgccaggagaaaatactagactgttat

SEQ ID NO: 1128 - DNA, 18 bp, Artificial Sequence
  primer
tctttgctcgcagctcgt

SEQ ID NO: 1129 - DNA, 21 bp, Artificial Sequence
  primer
ctaacactggagggcaccaaa

SEQ ID NO: 1130 - DNA, 19 bp, Artificial Sequence
  primer
ctgagaagccctgcccttc

SEQ ID NO: 1131 - DNA, 21 bp, Artificial Sequence
  primer
ctaacactggagggcaccaaa

SEQ ID NO: 1132 - DNA, 19 bp, Artificial Sequence
  primer
aaatacggctgcaccgagt

SEQ ID NO: 1133 - DNA, 21 bp, Artificial Sequence
  primer
ctaacactggagggcaccaaa

SEQ ID NO: 1134 - DNA, 24 bp, Artificial Sequence
  primer
ggtgatccagactctgaccttttg

SEQ ID NO: 1135 - DNA, 21 bp, Artificial Sequence
  primer
ctaacactggagggcaccaaa

SEQ ID NO: 1136 - DNA, 22 bp, Artificial Sequence
  primer
atcatgatggctgtatgtgcca

SEQ ID NO: 1137 - DNA, 21 bp, Artificial Sequence
  primer
ctaacactggagggcaccaaa

SEQ ID NO: 1138 - DNA, 21 bp, Artificial Sequence
  primer
catggttccaccagcgttatt

SEQ ID NO: 1139 - DNA, 21 bp, Artificial Sequence
  primer
ctaacactggagggcaccaaa

SEQ ID NO: 1140 - DNA, 18 bp, Artificial Sequence
  primer
tctttgctcgcagctcgt

SEQ ID NO: 1141 - DNA, 20 bp, Artificial Sequence
  primer
gggcgaactaagcctggttt

SEQ ID NO: 1142 - DNA, 19 bp, Artificial Sequence
  primer
ctgagaagccctgcccttc

SEQ ID NO: 1143 - DNA, 20 bp, Artificial Sequence
  primer
gggcgaactaagcctggttt

SEQ ID NO: 1144 - DNA, 19 bp, Artificial Sequence
  primer
aaatacggctgcaccgagt

SEQ ID NO: 1145 - DNA, 20 bp, Artificial Sequence
  primer
gggcgaactaagcctggttt

SEQ ID NO: 1146 - DNA, 24 bp, Artificial Sequence
  primer
ggtgatccagactctgaccttttg

SEQ ID NO: 1147 - DNA, 20 bp, Artificial Sequence
  primer
gggcgaactaagcctggttt

SEQ ID NO: 1148 - DNA, 22 bp, Artificial Sequence
  primer
atcatgatggctgtatgtgcca

SEQ ID NO: 1149 - DNA, 20 bp, Artificial Sequence
  primer
gggcgaactaagcctggttt

SEQ ID NO: 1150 - DNA, 21 bp, Artificial Sequence
  primer
catggttccaccagcgttatt

SEQ ID NO: 1151 - DNA, 20 bp, Artificial Sequence
  primer
gggcgaactaagcctggttt

SEQ ID NO: 1152 - DNA, 18 bp, Artificial Sequence
  primer
tctttgctcgcagctcgt

SEQ ID NO: 1153 - DNA, 28 bp, Artificial Sequence
  primer
gctgatagtaatgacctaaacgcacaaa

SEQ ID NO: 1154 - DNA, 19 bp, Artificial Sequence
  primer
ctgagaagccctgcccttc

SEQ ID NO: 1155 - DNA, 28 bp, Artificial Sequence
  primer
gctgatagtaatgacctaaacgcacaaa

SEQ ID NO: 1156 - DNA, 19 bp, Artificial Sequence
  primer
aaatacggctgcaccgagt

SEQ ID NO: 1157 - DNA, 28 bp, Artificial Sequence
  primer
gctgatagtaatgacctaaacgcacaaa

SEQ ID NO: 1158 - DNA, 24 bp, Artificial Sequence
  primer
ggtgatccagactctgaccttttg

SEQ ID NO: 1159 - DNA, 28 bp, Artificial Sequence
  primer
gctgatagtaatgacctaaacgcacaaa

SEQ ID NO: 1160 - DNA, 22 bp, Artificial Sequence
  primer
atcatgatggctgtatgtgcca

SEQ ID NO: 1161 - DNA, 28 bp, Artificial Sequence
  primer
gctgatagtaatgacctaaacgcacaaa

SEQ ID NO: 1162 - DNA, 21 bp, Artificial Sequence
  primer
catggttccaccagcgttatt

SEQ ID NO: 1163 - DNA, 28 bp, Artificial Sequence
  primer
gctgatagtaatgacctaaacgcacaaa

SEQ ID NO: 1164 - DNA, 18 bp, Artificial Sequence
  primer
tctttgctcgcagctcgt

SEQ ID NO: 1165 - DNA, 21 bp, Artificial Sequence
  primer
tcactgcaactgagtgcacaa

SEQ ID NO: 1166 - DNA, 19 bp, Artificial Sequence
  primer
ctgagaagccctgcccttc

SEQ ID NO: 1167 - DNA, 21 bp, Artificial Sequence
  primer
tcactgcaactgagtgcacaa

SEQ ID NO: 1168 - DNA, 19 bp, Artificial Sequence
  primer
aaatacggctgcaccgagt

SEQ ID NO: 1169 - DNA, 21 bp, Artificial Sequence
  primer
tcactgcaactgagtgcacaa

SEQ ID NO: 1170 - DNA, 24 bp, Artificial Sequence
  primer
ggtgatccagactctgaccttttg

SEQ ID NO: 1171 - DNA, 21 bp, Artificial Sequence
  primer
tcactgcaactgagtgcacaa

SEQ ID NO: 1172 - DNA, 22 bp, Artificial Sequence
  primer
atcatgatggctgtatgtgcca

SEQ ID NO: 1173 - DNA, 21 bp, Artificial Sequence
  primer
tcactgcaactgagtgcacaa

SEQ ID NO: 1174 - DNA, 21 bp, Artificial Sequence
  primer
catggttccaccagcgttatt

SEQ ID NO: 1175 - DNA, 21 bp, Artificial Sequence
  primer
tcactgcaactgagtgcacaa

SEQ ID NO: 1176 - DNA, 18 bp, Artificial Sequence
  primer
tctttgctcgcagctcgt

SEQ ID NO: 1177 - DNA, 22 bp, Artificial Sequence
  primer
gctgttgggcacattacaagtt

SEQ ID NO: 1178 - DNA, 19 bp, Artificial Sequence
  primer
ctgagaagccctgcccttc

SEQ ID NO: 1179 - DNA, 22 bp, Artificial Sequence
  primer
gctgttgggcacattacaagtt

SEQ ID NO: 1180 - DNA, 19 bp, Artificial Sequence
  primer
aaatacggctgcaccgagt

SEQ ID NO: 1181 - DNA, 22 bp, Artificial Sequence
  primer
gctgttgggcacattacaagtt

SEQ ID NO: 1182 - DNA, 24 bp, Artificial Sequence
  primer
ggtgatccagactctgaccttttg

SEQ ID NO: 1183 - DNA, 22 bp, Artificial Sequence
  primer
gctgttgggcacattacaagtt

SEQ ID NO: 1184 - DNA, 22 bp, Artificial Sequence
  primer
atcatgatggctgtatgtgcca

SEQ ID NO: 1185 - DNA, 22 bp, Artificial Sequence
  primer
gctgttgggcacattacaagtt

SEQ ID NO: 1186 - DNA, 21 bp, Artificial Sequence
  primer
catggttccaccagcgttatt

SEQ ID NO: 1187 - DNA, 22 bp, Artificial Sequence
  primer
gctgttgggcacattacaagtt

SEQ ID NO: 1188 - DNA, 18 bp, Artificial Sequence
  primer
tctttgctcgcagctcgt

SEQ ID NO: 1189 - DNA, 27 bp, Artificial Sequence
  primer
gtgccagaacaaaatactagactgttt

SEQ ID NO: 1190 - DNA, 19 bp, Artificial Sequence
  primer
ctgagaagccctgcccttc

SEQ ID NO: 1191 - DNA, 27 bp, Artificial Sequence
  primer
gtgccagaacaaaatactagactgttt

SEQ ID NO: 1192 - DNA, 19 bp, Artificial Sequence
  primer
aaatacggctgcaccgagt

SEQ ID NO: 1193 - DNA, 27 bp, Artificial Sequence
  primer
gtgccagaacaaaatactagactgttt

SEQ ID NO: 1194 - DNA, 24 bp, Artificial Sequence
  primer
ggtgatccagactctgaccttttg

SEQ ID NO: 1195 - DNA, 27 bp, Artificial Sequence
  primer
gtgccagaacaaaatactagactgttt

SEQ ID NO: 1196 - DNA, 22 bp, Artificial Sequence
  primer
atcatgatggctgtatgtgcca

SEQ ID NO: 1197 - DNA, 27 bp, Artificial Sequence
  primer
gtgccagaacaaaatactagactgttt

SEQ ID NO: 1198 - DNA, 21 bp, Artificial Sequence
  primer
catggttccaccagcgttatt

SEQ ID NO: 1199 - DNA, 27 bp, Artificial Sequence
  primer
gtgccagaacaaaatactagactgttt

SEQ ID NO: 1200 - DNA, 18 bp, Artificial Sequence
  primer
tctttgctcgcagctcgt

SEQ ID NO: 1201 - DNA, 23 bp, Artificial Sequence
  primer
acaacaaccaccctggtgataag

SEQ ID NO: 1202 - DNA, 19 bp, Artificial Sequence
  primer
ctgagaagccctgcccttc

SEQ ID NO: 1203 - DNA, 23 bp, Artificial Sequence
  primer
acaacaaccaccctggtgataag

SEQ ID NO: 1204 - DNA, 19 bp, Artificial Sequence
  primer
aaatacggctgcaccgagt

SEQ ID NO: 1205 - DNA, 23 bp, Artificial Sequence
  primer
acaacaaccaccctggtgataag

SEQ ID NO: 1206 - DNA, 24 bp, Artificial Sequence
  primer
ggtgatccagactctgaccttttg

SEQ ID NO: 1207 - DNA, 23 bp, Artificial Sequence
  primer
acaacaaccaccctggtgataag

SEQ ID NO: 1208 - DNA, 22 bp, Artificial Sequence
  primer
atcatgatggctgtatgtgcca

SEQ ID NO: 1209 - DNA, 23 bp, Artificial Sequence
  primer
acaacaaccaccctggtgataag

SEQ ID NO: 1210 - DNA, 21 bp, Artificial Sequence
  primer
catggttccaccagcgttatt

SEQ ID NO: 1211 - DNA, 23 bp, Artificial Sequence
  primer
acaacaaccaccctggtgataag

SEQ ID NO: 1212 - DNA, 18 bp, Artificial Sequence
  primer
tctttgctcgcagctcgt

SEQ ID NO: 1213 - DNA, 23 bp, Artificial Sequence
  primer
gttaacagtaacgtgcccactct

SEQ ID NO: 1214 - DNA, 19 bp, Artificial Sequence
  primer
ctgagaagccctgcccttc

SEQ ID NO: 1215 - DNA, 23 bp, Artificial Sequence
  primer
gttaacagtaacgtgcccactct

SEQ ID NO: 1216 - DNA, 19 bp, Artificial Sequence
  primer
aaatacggctgcaccgagt

SEQ ID NO: 1217 - DNA, 23 bp, Artificial Sequence
  primer
gttaacagtaacgtgcccactct

SEQ ID NO: 1218 - DNA, 24 bp, Artificial Sequence
  primer
ggtgatccagactctgaccttttg

SEQ ID NO: 1219 - DNA, 23 bp, Artificial Sequence
  primer
gttaacagtaacgtgcccactct

SEQ ID NO: 1220 - DNA, 22 bp, Artificial Sequence
  primer
atcatgatggctgtatgtgcca

SEQ ID NO: 1221 - DNA, 23 bp, Artificial Sequence
  primer
gttaacagtaacgtgcccactct

SEQ ID NO: 1222 - DNA, 21 bp, Artificial Sequence
  primer
catggttccaccagcgttatt

SEQ ID NO: 1223 - DNA, 23 bp, Artificial Sequence
  primer
gttaacagtaacgtgcccactct

SEQ ID NO: 1224 - DNA, 18 bp, Artificial Sequence
  primer
tctttgctcgcagctcgt

SEQ ID NO: 1225 - DNA, 28 bp, Artificial Sequence
  primer
gcaggacaaaatcctagacatatacgaa

SEQ ID NO: 1226 - DNA, 19 bp, Artificial Sequence
  primer
ctgagaagccctgcccttc

SEQ ID NO: 1227 - DNA, 28 bp, Artificial Sequence
  primer
gcaggacaaaatcctagacatatacgaa

SEQ ID NO: 1228 - DNA, 19 bp, Artificial Sequence
  primer
aaatacggctgcaccgagt

SEQ ID NO: 1229 - DNA, 28 bp, Artificial Sequence
  primer
gcaggacaaaatcctagacatatacgaa

SEQ ID NO: 1230 - DNA, 24 bp, Artificial Sequence
  primer
ggtgatccagactctgaccttttg

SEQ ID NO: 1231 - DNA, 28 bp, Artificial Sequence
  primer
gcaggacaaaatcctagacatatacgaa

SEQ ID NO: 1232 - DNA, 22 bp, Artificial Sequence
  primer
atcatgatggctgtatgtgcca

SEQ ID NO: 1233 - DNA, 28 bp, Artificial Sequence
  primer
gcaggacaaaatcctagacatatacgaa

SEQ ID NO: 1234 - DNA, 21 bp, Artificial Sequence
  primer
catggttccaccagcgttatt

SEQ ID NO: 1235 - DNA, 28 bp, Artificial Sequence
  primer
gcaggacaaaatcctagacatatacgaa

SEQ ID NO: 1236 - DNA, 18 bp, Artificial Sequence
  primer
tctttgctcgcagctcgt

SEQ ID NO: 1237 - DNA, 27 bp, Artificial Sequence
  primer
gaggaggactacacagtacaactaact

SEQ ID NO: 1238 - DNA, 19 bp, Artificial Sequence
  primer
ctgagaagccctgcccttc

SEQ ID NO: 1239 - DNA, 27 bp, Artificial Sequence
  primer
gaggaggactacacagtacaactaact

SEQ ID NO: 1240 - DNA, 19 bp, Artificial Sequence
  primer
aaatacggctgcaccgagt

SEQ ID NO: 1241 - DNA, 27 bp, Artificial Sequence
  primer
gaggaggactacacagtacaactaact

SEQ ID NO: 1242 - DNA, 24 bp, Artificial Sequence
  primer
ggtgatccagactctgaccttttg

SEQ ID NO: 1243 - DNA, 27 bp, Artificial Sequence
  primer
gaggaggactacacagtacaactaact

SEQ ID NO: 1244 - DNA, 22 bp, Artificial Sequence
  primer
atcatgatggctgtatgtgcca

SEQ ID NO: 1245 - DNA, 27 bp, Artificial Sequence
  primer
gaggaggactacacagtacaactaact

SEQ ID NO: 1246 - DNA, 21 bp, Artificial Sequence
  primer
catggttccaccagcgttatt

SEQ ID NO: 1247 - DNA, 27 bp, Artificial Sequence
  primer
gaggaggactacacagtacaactaact

SEQ ID NO: 1248 - DNA, 18 bp, Artificial Sequence
  primer
tctttgctcgcagctcgt

SEQ ID NO: 1249 - DNA, 23 bp, Artificial Sequence
  primer
tgcttatgggcacatgtaccatt

SEQ ID NO: 1250 - DNA, 19 bp, Artificial Sequence
  primer
ctgagaagccctgcccttc

SEQ ID NO: 1251 - DNA, 23 bp, Artificial Sequence
  primer
tgcttatgggcacatgtaccatt

SEQ ID NO: 1252 - DNA, 19 bp, Artificial Sequence
  primer
aaatacggctgcaccgagt

SEQ ID NO: 1253 - DNA, 23 bp, Artificial Sequence
  primer
tgcttatgggcacatgtaccatt

SEQ ID NO: 1254 - DNA, 24 bp, Artificial Sequence
  primer
ggtgatccagactctgaccttttg

SEQ ID NO: 1255 - DNA, 23 bp, Artificial Sequence
  primer
tgcttatgggcacatgtaccatt

SEQ ID NO: 1256 - DNA, 22 bp, Artificial Sequence
  primer
atcatgatggctgtatgtgcca

SEQ ID NO: 1257 - DNA, 23 bp, Artificial Sequence
  primer
tgcttatgggcacatgtaccatt

SEQ ID NO: 1258 - DNA, 21 bp, Artificial Sequence
  primer
catggttccaccagcgttatt

SEQ ID NO: 1259 - DNA, 23 bp, Artificial Sequence
  primer
tgcttatgggcacatgtaccatt

SEQ ID NO: 1260 - DNA, 18 bp, Artificial Sequence
  primer
tctttgctcgcagctcgt

SEQ ID NO: 1261 - DNA, 27 bp, Artificial Sequence
  primer
gcgtttaagtgtgttacaggatcaaat

SEQ ID NO: 1262 - DNA, 19 bp, Artificial Sequence
  primer
ctgagaagccctgcccttc

SEQ ID NO: 1263 - DNA, 27 bp, Artificial Sequence
  primer
gcgtttaagtgtgttacaggatcaaat

SEQ ID NO: 1264 - DNA, 19 bp, Artificial Sequence
  primer
aaatacggctgcaccgagt

SEQ ID NO: 1265 - DNA, 27 bp, Artificial Sequence
  primer
gcgtttaagtgtgttacaggatcaaat

SEQ ID NO: 1266 - DNA, 24 bp, Artificial Sequence
  primer
ggtgatccagactctgaccttttg

SEQ ID NO: 1267 - DNA, 27 bp, Artificial Sequence
  primer
gcgtttaagtgtgttacaggatcaaat

SEQ ID NO: 1268 - DNA, 22 bp, Artificial Sequence
  primer
atcatgatggctgtatgtgcca

SEQ ID NO: 1269 - DNA, 27 bp, Artificial Sequence
  primer
gcgtttaagtgtgttacaggatcaaat

SEQ ID NO: 1270 - DNA, 21 bp, Artificial Sequence
  primer
catggttccaccagcgttatt

SEQ ID NO: 1271 - DNA, 27 bp, Artificial Sequence
  primer
gcgtttaagtgtgttacaggatcaaat

SEQ ID NO: 1272 - DNA, 18 bp, Artificial Sequence
  primer
tctttgctcgcagctcgt

SEQ ID NO: 1273 - DNA, 19 bp, Artificial Sequence
  primer
tccgtttgcatccaggcaa

SEQ ID NO: 1274 - DNA, 19 bp, Artificial Sequence
  primer
ctgagaagccctgcccttc

SEQ ID NO: 1275 - DNA, 19 bp, Artificial Sequence
  primer
tccgtttgcatccaggcaa

SEQ ID NO: 1276 - DNA, 19 bp, Artificial Sequence
  primer
aaatacggctgcaccgagt

SEQ ID NO: 1277 - DNA, 19 bp, Artificial Sequence
  primer
tccgtttgcatccaggcaa

SEQ ID NO: 1278 - DNA, 24 bp, Artificial Sequence
  primer
ggtgatccagactctgaccttttg

SEQ ID NO: 1279 - DNA, 19 bp, Artificial Sequence
  primer
tccgtttgcatccaggcaa

SEQ ID NO: 1280 - DNA, 22 bp, Artificial Sequence
  primer
atcatgatggctgtatgtgcca

SEQ ID NO: 1281 - DNA, 19 bp, Artificial Sequence
  primer
tccgtttgcatccaggcaa

SEQ ID NO: 1282 - DNA, 21 bp, Artificial Sequence
  primer
catggttccaccagcgttatt

SEQ ID NO: 1283 - DNA, 19 bp, Artificial Sequence
  primer
tccgtttgcatccaggcaa

SEQ ID NO: 1284 - DNA, 18 bp, Artificial Sequence
  primer
tctttgctcgcagctcgt

SEQ ID NO: 1285 - DNA, 25 bp, Artificial Sequence
  primer
actatcctttgtgtgtcctttgtgt

SEQ ID NO: 1286 - DNA, 19 bp, Artificial Sequence
  primer
ctgagaagccctgcccttc

SEQ ID NO: 1287 - DNA, 25 bp, Artificial Sequence
  primer
actatcctttgtgtgtcctttgtgt

SEQ ID NO: 1288 - DNA, 19 bp, Artificial Sequence
  primer
aaatacggctgcaccgagt

SEQ ID NO: 1289 - DNA, 25 bp, Artificial Sequence
  primer
actatcctttgtgtgtcctttgtgt

SEQ ID NO: 1290 - DNA, 24 bp, Artificial Sequence
  primer
ggtgatccagactctgaccttttg

SEQ ID NO: 1291 - DNA, 25 bp, Artificial Sequence
  primer
actatcctttgtgtgtcctttgtgt

SEQ ID NO: 1292 - DNA, 22 bp, Artificial Sequence
  primer
atcatgatggctgtatgtgcca

SEQ ID NO: 1293 - DNA, 25 bp, Artificial Sequence
  primer
actatcctttgtgtgtcctttgtgt

SEQ ID NO: 1294 - DNA, 21 bp, Artificial Sequence
  primer
catggttccaccagcgttatt

SEQ ID NO: 1295 - DNA, 25 bp, Artificial Sequence
  primer
actatcctttgtgtgtcctttgtgt

SEQ ID NO: 1296 - DNA, 18 bp, Artificial Sequence
  primer
tctttgctcgcagctcgt

SEQ ID NO: 1297 - DNA, 26 bp, Artificial Sequence
  primer
cgtgccagaacaaaatactagactgt

SEQ ID NO: 1298 - DNA, 19 bp, Artificial Sequence
  primer
ctgagaagccctgcccttc

SEQ ID NO: 1299 - DNA, 26 bp, Artificial Sequence
  primer
cgtgccagaacaaaatactagactgt

SEQ ID NO: 1300 - DNA, 19 bp, Artificial Sequence
  primer
aaatacggctgcaccgagt

SEQ ID NO: 1301 - DNA, 26 bp, Artificial Sequence
  primer
cgtgccagaacaaaatactagactgt

SEQ ID NO: 1302 - DNA, 24 bp, Artificial Sequence
  primer
ggtgatccagactctgaccttttg

SEQ ID NO: 1303 - DNA, 26 bp, Artificial Sequence
  primer
cgtgccagaacaaaatactagactgt

SEQ ID NO: 1304 - DNA, 22 bp, Artificial Sequence
  primer
atcatgatggctgtatgtgcca

SEQ ID NO: 1305 - DNA, 26 bp, Artificial Sequence
  primer
cgtgccagaacaaaatactagactgt

SEQ ID NO: 1306 - DNA, 21 bp, Artificial Sequence
  primer
catggttccaccagcgttatt

SEQ ID NO: 1307 - DNA, 26 bp, Artificial Sequence
  primer
cgtgccagaacaaaatactagactgt

SEQ ID NO: 1308 - DNA, 18 bp, Artificial Sequence
  primer
tctttgctcgcagctcgt

SEQ ID NO: 1309 - DNA, 23 bp, Artificial Sequence
  primer
gtatcaacacacaaagccactgt

SEQ ID NO: 1310 - DNA, 19 bp, Artificial Sequence
  primer
ctgagaagccctgcccttc

SEQ ID NO: 1311 - DNA, 23 bp, Artificial Sequence
  primer
gtatcaacacacaaagccactgt

SEQ ID NO: 1312 - DNA, 19 bp, Artificial Sequence
  primer
aaatacggctgcaccgagt

SEQ ID NO: 1313 - DNA, 23 bp, Artificial Sequence
  primer
gtatcaacacacaaagccactgt

SEQ ID NO: 1314 - DNA, 24 bp, Artificial Sequence
  primer
ggtgatccagactctgaccttttg

SEQ ID NO: 1315 - DNA, 23 bp, Artificial Sequence
  primer
gtatcaacacacaaagccactgt

SEQ ID NO: 1316 - DNA, 22 bp, Artificial Sequence
  primer
atcatgatggctgtatgtgcca

SEQ ID NO: 1317 - DNA, 23 bp, Artificial Sequence
  primer
gtatcaacacacaaagccactgt

SEQ ID NO: 1318 - DNA, 21 bp, Artificial Sequence
  primer
catggttccaccagcgttatt

SEQ ID NO: 1319 - DNA, 23 bp, Artificial Sequence
  primer
gtatcaacacacaaagccactgt

SEQ ID NO: 1320 - DNA, 18 bp, Artificial Sequence
  primer
tctttgctcgcagctcgt

SEQ ID NO: 1321 - DNA, 23 bp, Artificial Sequence
  primer
gttaacagtaacgtgcccactct

SEQ ID NO: 1322 - DNA, 19 bp, Artificial Sequence
  primer
ctgagaagccctgcccttc

SEQ ID NO: 1323 - DNA, 23 bp, Artificial Sequence
  primer
gttaacagtaacgtgcccactct

SEQ ID NO: 1324 - DNA, 19 bp, Artificial Sequence
  primer
aaatacggctgcaccgagt

SEQ ID NO: 1325 - DNA, 23 bp, Artificial Sequence
  primer
gttaacagtaacgtgcccactct

SEQ ID NO: 1326 - DNA, 24 bp, Artificial Sequence
  primer
ggtgatccagactctgaccttttg

SEQ ID NO: 1327 - DNA, 23 bp, Artificial Sequence
  primer
gttaacagtaacgtgcccactct

SEQ ID NO: 1328 - DNA, 22 bp, Artificial Sequence
  primer
atcatgatggctgtatgtgcca

SEQ ID NO: 1329 - DNA, 23 bp, Artificial Sequence
  primer
gttaacagtaacgtgcccactct

SEQ ID NO: 1330 - DNA, 21 bp, Artificial Sequence
  primer
catggttccaccagcgttatt

SEQ ID NO: 1331 - DNA, 23 bp, Artificial Sequence
  primer
gttaacagtaacgtgcccactct

SEQ ID NO: 1332 - DNA, 18 bp, Artificial Sequence
  primer
tctttgctcgcagctcgt

SEQ ID NO: 1333 - DNA, 28 bp, Artificial Sequence
  primer
acaggacagtaaatgtatacaggaccat

SEQ ID NO: 1334 - DNA, 19 bp, Artificial Sequence
  primer
ctgagaagccctgcccttc

SEQ ID NO: 1335 - DNA, 28 bp, Artificial Sequence
  primer
acaggacagtaaatgtatacaggaccat

SEQ ID NO: 1336 - DNA, 19 bp, Artificial Sequence
  primer
aaatacggctgcaccgagt

SEQ ID NO: 1337 - DNA, 28 bp, Artificial Sequence
  primer
acaggacagtaaatgtatacaggaccat

SEQ ID NO: 1338 - DNA, 24 bp, Artificial Sequence
  primer
ggtgatccagactctgaccttttg

SEQ ID NO: 1339 - DNA, 28 bp, Artificial Sequence
  primer
acaggacagtaaatgtatacaggaccat

SEQ ID NO: 1340 - DNA, 22 bp, Artificial Sequence
  primer
atcatgatggctgtatgtgcca

SEQ ID NO: 1341 - DNA, 28 bp, Artificial Sequence
  primer
acaggacagtaaatgtatacaggaccat

SEQ ID NO: 1342 - DNA, 21 bp, Artificial Sequence
  primer
catggttccaccagcgttatt

SEQ ID NO: 1343 - DNA, 28 bp, Artificial Sequence
  primer
acaggacagtaaatgtatacaggaccat

SEQ ID NO: 1344 - DNA, 18 bp, Artificial Sequence
  primer
tctttgctcgcagctcgt

SEQ ID NO: 1345 - DNA, 23 bp, Artificial Sequence
  primer
agtagaagtgcaggccaaaacaa

SEQ ID NO: 1346 - DNA, 19 bp, Artificial Sequence
  primer
ctgagaagccctgcccttc

SEQ ID NO: 1347 - DNA, 23 bp, Artificial Sequence
  primer
agtagaagtgcaggccaaaacaa

SEQ ID NO: 1348 - DNA, 19 bp, Artificial Sequence
  primer
aaatacggctgcaccgagt

SEQ ID NO: 1349 - DNA, 23 bp, Artificial Sequence
  primer
agtagaagtgcaggccaaaacaa

SEQ ID NO: 1350 - DNA, 24 bp, Artificial Sequence
  primer
ggtgatccagactctgaccttttg

SEQ ID NO: 1351 - DNA, 23 bp, Artificial Sequence
  primer
agtagaagtgcaggccaaaacaa

SEQ ID NO: 1352 - DNA, 22 bp, Artificial Sequence
  primer
atcatgatggctgtatgtgcca

SEQ ID NO: 1353 - DNA, 23 bp, Artificial Sequence
  primer
agtagaagtgcaggccaaaacaa

SEQ ID NO: 1354 - DNA, 21 bp, Artificial Sequence
  primer
catggttccaccagcgttatt

SEQ ID NO: 1355 - DNA, 23 bp, Artificial Sequence
  primer
agtagaagtgcaggccaaaacaa

SEQ ID NO: 1356 - DNA, 18 bp, Artificial Sequence
  primer
tctttgctcgcagctcgt

SEQ ID NO: 1357 - DNA, 19 bp, Artificial Sequence
  primer
tccgtggtgtgcaactgaa

SEQ ID NO: 1358 - DNA, 19 bp, Artificial Sequence
  primer
ctgagaagccctgcccttc

SEQ ID NO: 1359 - DNA, 19 bp, Artificial Sequence
  primer
tccgtggtgtgcaactgaa

SEQ ID NO: 1360 - DNA, 19 bp, Artificial Sequence
  primer
aaatacggctgcaccgagt

SEQ ID NO: 1361 - DNA, 19 bp, Artificial Sequence
  primer
tccgtggtgtgcaactgaa

SEQ ID NO: 1362 - DNA, 24 bp, Artificial Sequence
  primer
ggtgatccagactctgaccttttg

SEQ ID NO: 1363 - DNA, 19 bp, Artificial Sequence
  primer
tccgtggtgtgcaactgaa

SEQ ID NO: 1364 - DNA, 22 bp, Artificial Sequence
  primer
atcatgatggctgtatgtgcca

SEQ ID NO: 1365 - DNA, 19 bp, Artificial Sequence
  primer
tccgtggtgtgcaactgaa

SEQ ID NO: 1366 - DNA, 21 bp, Artificial Sequence
  primer
catggttccaccagcgttatt

SEQ ID NO: 1367 - DNA, 19 bp, Artificial Sequence
  primer
tccgtggtgtgcaactgaa

SEQ ID NO: 1368 - DNA, 18 bp, Artificial Sequence
  primer
tctttgctcgcagctcgt

SEQ ID NO: 1369 - DNA, 26 bp, Artificial Sequence
  primer
gtatgaacgtgacagtgtacacctaa

SEQ ID NO: 1370 - DNA, 19 bp, Artificial Sequence
  primer
ctgagaagccctgcccttc

SEQ ID NO: 1371 - DNA, 26 bp, Artificial Sequence
  primer
gtatgaacgtgacagtgtacacctaa

SEQ ID NO: 1372 - DNA, 19 bp, Artificial Sequence
  primer
aaatacggctgcaccgagt

SEQ ID NO: 1373 - DNA, 26 bp, Artificial Sequence
  primer
gtatgaacgtgacagtgtacacctaa

SEQ ID NO: 1374 - DNA, 24 bp, Artificial Sequence
  primer
ggtgatccagactctgaccttttg

SEQ ID NO: 1375 - DNA, 26 bp, Artificial Sequence
  primer
gtatgaacgtgacagtgtacacctaa

SEQ ID NO: 1376 - DNA, 22 bp, Artificial Sequence
  primer
atcatgatggctgtatgtgcca

SEQ ID NO: 1377 - DNA, 26 bp, Artificial Sequence
  primer
gtatgaacgtgacagtgtacacctaa

SEQ ID NO: 1378 - DNA, 21 bp, Artificial Sequence
  primer
catggttccaccagcgttatt

SEQ ID NO: 1379 - DNA, 26 bp, Artificial Sequence
  primer
gtatgaacgtgacagtgtacacctaa

SEQ ID NO: 1380 - DNA, 18 bp, Artificial Sequence
  primer
tctttgctcgcagctcgt

SEQ ID NO: 1381 - DNA, 21 bp, Artificial Sequence
  primer
acctacatcccaccacagagt

SEQ ID NO: 1382 - DNA, 19 bp, Artificial Sequence
  primer
ctgagaagccctgcccttc

SEQ ID NO: 1383 - DNA, 21 bp, Artificial Sequence
  primer
acctacatcccaccacagagt

SEQ ID NO: 1384 - DNA, 19 bp, Artificial Sequence
  primer
aaatacggctgcaccgagt

SEQ ID NO: 1385 - DNA, 21 bp, Artificial Sequence
  primer
acctacatcccaccacagagt

SEQ ID NO: 1386 - DNA, 24 bp, Artificial Sequence
  primer
ggtgatccagactctgaccttttg

SEQ ID NO: 1387 - DNA, 21 bp, Artificial Sequence
  primer
acctacatcccaccacagagt

SEQ ID NO: 1388 - DNA, 22 bp, Artificial Sequence
  primer
atcatgatggctgtatgtgcca

SEQ ID NO: 1389 - DNA, 21 bp, Artificial Sequence
  primer
acctacatcccaccacagagt

SEQ ID NO: 1390 - DNA, 21 bp, Artificial Sequence
  primer
catggttccaccagcgttatt

SEQ ID NO: 1391 - DNA, 21 bp, Artificial Sequence
  primer
acctacatcccaccacagagt

SEQ ID NO: 1392 - DNA, 18 bp, Artificial Sequence
  primer
tctttgctcgcagctcgt

SEQ ID NO: 1393 - DNA, 27 bp, Artificial Sequence
  primer
tgcttatgggtacactaggtattgtgt

SEQ ID NO: 1394 - DNA, 19 bp, Artificial Sequence
  primer
ctgagaagccctgcccttc

SEQ ID NO: 1395 - DNA, 27 bp, Artificial Sequence
  primer
tgcttatgggtacactaggtattgtgt

SEQ ID NO: 1396 - DNA, 19 bp, Artificial Sequence
  primer
aaatacggctgcaccgagt

SEQ ID NO: 1397 - DNA, 27 bp, Artificial Sequence
  primer
tgcttatgggtacactaggtattgtgt

SEQ ID NO: 1398 - DNA, 24 bp, Artificial Sequence
  primer
ggtgatccagactctgaccttttg

SEQ ID NO: 1399 - DNA, 27 bp, Artificial Sequence
  primer
tgcttatgggtacactaggtattgtgt

SEQ ID NO: 1400 - DNA, 22 bp, Artificial Sequence
  primer
atcatgatggctgtatgtgcca

SEQ ID NO: 1401 - DNA, 27 bp, Artificial Sequence
  primer
tgcttatgggtacactaggtattgtgt

SEQ ID NO: 1402 - DNA, 21 bp, Artificial Sequence
  primer
catggttccaccagcgttatt

SEQ ID NO: 1403 - DNA, 27 bp, Artificial Sequence
  primer
tgcttatgggtacactaggtattgtgt

SEQ ID NO: 1404 - DNA, 18 bp, Artificial Sequence
  primer
tctttgctcgcagctcgt

SEQ ID NO: 1405 - DNA, 28 bp, Artificial Sequence
  primer
gtgccaggagaaaatactagactgttat

SEQ ID NO: 1406 - DNA, 19 bp, Artificial Sequence
  primer
ctgagaagccctgcccttc

SEQ ID NO: 1407 - DNA, 28 bp, Artificial Sequence
  primer
gtgccaggagaaaatactagactgttat

SEQ ID NO: 1408 - DNA, 19 bp, Artificial Sequence
  primer
aaatacggctgcaccgagt

SEQ ID NO: 1409 - DNA, 28 bp, Artificial Sequence
  primer
gtgccaggagaaaatactagactgttat

SEQ ID NO: 1410 - DNA, 24 bp, Artificial Sequence
  primer
ggtgatccagactctgaccttttg

SEQ ID NO: 1411 - DNA, 28 bp, Artificial Sequence
  primer
gtgccaggagaaaatactagactgttat

SEQ ID NO: 1412 - DNA, 22 bp, Artificial Sequence
  primer
atcatgatggctgtatgtgcca

SEQ ID NO: 1413 - DNA, 28 bp, Artificial Sequence
  primer
gtgccaggagaaaatactagactgttat

SEQ ID NO: 1414 - DNA, 21 bp, Artificial Sequence
  primer
catggttccaccagcgttatt

SEQ ID NO: 1415 - DNA, 28 bp, Artificial Sequence
  primer
gtgccaggagaaaatactagactgttat

SEQ ID NO: 1416 - DNA, 18 bp, Artificial Sequence
  primer
tctttgctcgcagctcgt

SEQ ID NO: 1417 - DNA, 21 bp, Artificial Sequence
  primer
tgcgaccaccaaatacactgt

SEQ ID NO: 1418 - DNA, 19 bp, Artificial Sequence
  primer
ctgagaagccctgcccttc

SEQ ID NO: 1419 - DNA, 21 bp, Artificial Sequence
  primer
tgcgaccaccaaatacactgt

SEQ ID NO: 1420 - DNA, 19 bp, Artificial Sequence
  primer
aaatacggctgcaccgagt

SEQ ID NO: 1421 - DNA, 21 bp, Artificial Sequence
  primer
tgcgaccaccaaatacactgt

SEQ ID NO: 1422 - DNA, 24 bp, Artificial Sequence
  primer
ggtgatccagactctgaccttttg

SEQ ID NO: 1423 - DNA, 21 bp, Artificial Sequence
  primer
tgcgaccaccaaatacactgt

SEQ ID NO: 1424 - DNA, 22 bp, Artificial Sequence
  primer
atcatgatggctgtatgtgcca

SEQ ID NO: 1425 - DNA, 21 bp, Artificial Sequence
  primer
tgcgaccaccaaatacactgt

SEQ ID NO: 1426 - DNA, 21 bp, Artificial Sequence
  primer
catggttccaccagcgttatt

SEQ ID NO: 1427 - DNA, 21 bp, Artificial Sequence
  primer
tgcgaccaccaaatacactgt

SEQ ID NO: 1428 - DNA, 18 bp, Artificial Sequence
  primer
tctttgctcgcagctcgt

SEQ ID NO: 1429 - DNA, 20 bp, Artificial Sequence
  primer
cgtggtgtgcgaccaactaa

SEQ ID NO: 1430 - DNA, 19 bp, Artificial Sequence
  primer
ctgagaagccctgcccttc

SEQ ID NO: 1431 - DNA, 20 bp, Artificial Sequence
  primer
cgtggtgtgcgaccaactaa

SEQ ID NO: 1432 - DNA, 19 bp, Artificial Sequence
  primer
aaatacggctgcaccgagt

SEQ ID NO: 1433 - DNA, 20 bp, Artificial Sequence
  primer
cgtggtgtgcgaccaactaa

SEQ ID NO: 1434 - DNA, 24 bp, Artificial Sequence
  primer
ggtgatccagactctgaccttttg

SEQ ID NO: 1435 - DNA, 20 bp, Artificial Sequence
  primer
cgtggtgtgcgaccaactaa

SEQ ID NO: 1436 - DNA, 22 bp, Artificial Sequence
  primer
atcatgatggctgtatgtgcca

SEQ ID NO: 1437 - DNA, 20 bp, Artificial Sequence
  primer
cgtggtgtgcgaccaactaa

SEQ ID NO: 1438 - DNA, 21 bp, Artificial Sequence
  primer
catggttccaccagcgttatt

SEQ ID NO: 1439 - DNA, 20 bp, Artificial Sequence
  primer
cgtggtgtgcgaccaactaa

SEQ ID NO: 1440 - DNA, 18 bp, Artificial Sequence
  primer
tctttgctcgcagctcgt

SEQ ID NO: 1441 - DNA, 22 bp, Artificial Sequence
  primer
ccaggtcatcaccattggcaat

SEQ ID NO: 1442 - DNA, 21 bp, Artificial Sequence
  primer
cgtacaggtctttgcggatgt

SEQ ID NO: 1443 - DNA, 20 bp, Artificial Sequence
  primer
ccatgagcgacgtggctatt

SEQ ID NO: 1444 - DNA, 20 bp, Artificial Sequence
  primer
ctcacgttggtccacatcct

SEQ ID NO: 1445 - DNA, 18 bp, Artificial Sequence
  primer
ctgtgctcgcgctactct

SEQ ID NO: 1446 - DNA, 26 bp, Artificial Sequence
  primer
caacttcaatgtcggatggatgaaac

SEQ ID NO: 1447 - DNA, 23 bp, Artificial Sequence
  primer
gtggatgactgagtacctgaacc

SEQ ID NO: 1448 - DNA, 21 bp, Artificial Sequence
  primer
ggccaaactgagcagagtctt

SEQ ID NO: 1449 - DNA, 21 bp, Artificial Sequence
  primer
cgggactcgagtgatgattgg

SEQ ID NO: 1450 - DNA, 20 bp, Artificial Sequence
  primer
ctgaggtgtaggtgctgtca

SEQ ID NO: 1451 - DNA, 24 bp, Artificial Sequence
  primer
ctcctgaaaagagagtggaagtgt

SEQ ID NO: 1452 - DNA, 22 bp, Artificial Sequence
  primer
ccggattaatctccagccagtt

SEQ ID NO: 1453 - DNA, 21 bp, Artificial Sequence
  primer
aacgcaccgaatagttacggt

SEQ ID NO: 1454 - DNA, 17 bp, Artificial Sequence
  primer
acgggtcgggtgagagt

SEQ ID NO: 1455 - DNA, 19 bp, Artificial Sequence
  primer
cggatcccaacggagtcaa

SEQ ID NO: 1456 - DNA, 17 bp, Artificial Sequence
  primer
accggtcgggtgagagt

SEQ ID NO: 1457 - DNA, 24 bp, Artificial Sequence
  primer
tcttccagaacctgcaagtaatcc

SEQ ID NO: 1458 - DNA, 21 bp, Artificial Sequence
  primer
ggtgggtgttatggtggatga

SEQ ID NO: 1459 - DNA, 25 bp, Artificial Sequence
  primer
aggagaatccgaagggaaaggaata

SEQ ID NO: 1460 - DNA, 20 bp, Artificial Sequence
  primer
tccttcagcaggttggcaat

SEQ ID NO: 1461 - DNA, 19 bp, Artificial Sequence
  primer
agtccactggcgtcttcac

SEQ ID NO: 1462 - DNA, 26 bp, Artificial Sequence
  primer
tgatcttgaggctgttgtcatacttc

SEQ ID NO: 1463 - DNA, 21 bp, Artificial Sequence
  primer
gcgagtatggagcagaaacga

SEQ ID NO: 1464 - DNA, 24 bp, Artificial Sequence
  primer
aattccaaatgagctctccaacca

SEQ ID NO: 1465 - DNA, 22 bp, Artificial Sequence
  primer
cggaatataagctggtggtggt

SEQ ID NO: 1466 - DNA, 20 bp, Artificial Sequence
  primer
gcacgtctccccatcaatga

SEQ ID NO: 1467 - DNA, 21 bp, Artificial Sequence
  primer
gtgcaatgagggaccagtaca

SEQ ID NO: 1468 - DNA, 28 bp, Artificial Sequence
  primer
ctactaggaccataggtacatcttcaga

SEQ ID NO: 1469 - DNA, 20 bp, Artificial Sequence
  primer
gatgagctgaccctgaccaa

SEQ ID NO: 1470 - DNA, 24 bp, Artificial Sequence
  primer
ggcagcattcatttccacattcac

SEQ ID NO: 1471 - DNA, 19 bp, Artificial Sequence
  primer
aggagctggcctacctgaa

SEQ ID NO: 1472 - DNA, 23 bp, Artificial Sequence
  primer
cttctcatactggtcacgcatct

SEQ ID NO: 1473 - DNA, 21 bp, Artificial Sequence
  primer
aacactgagctggaggtgaag

SEQ ID NO: 1474 - DNA, 22 bp, Artificial Sequence
  primer
ctgtagcaggatgttggcattg

SEQ ID NO: 1475 - DNA, 28 bp, Artificial Sequence
  primer
tgtgtgcattccctatcaaatatgtcaa

SEQ ID NO: 1476 - DNA, 19 bp, Artificial Sequence
  primer
gcgcttcacagcctgatga

SEQ ID NO: 1477 - DNA, 23 bp, Artificial Sequence
  primer
cgtcgtgtctcaagatctagctt

SEQ ID NO: 1478 - DNA, 22 bp, Artificial Sequence
  primer
tgagtcatctgcggtactgtct

SEQ ID NO: 1479 - DNA, 22 bp, Artificial Sequence
  primer
gcttctctgaaaggctctcctt

SEQ ID NO: 1480 - DNA, 19 bp, Artificial Sequence
  primer
aaatacggctgcaccgagt

SEQ ID NO: 1481 - DNA, 19 bp, Artificial Sequence
  primer
ccgacgcacaaggtgtctt

SEQ ID NO: 1482 - DNA, 20 bp, Artificial Sequence
  primer
gtcggcgtgtgagttgatga

SEQ ID NO: 1483 - DNA, 24 bp, Artificial Sequence
  primer
gacggagtgaaattttctgcaagt

SEQ ID NO: 1484 - DNA, 24 bp, Artificial Sequence
  primer
gaagttcaggtacctcagtgcaaa

SEQ ID NO: 1485 - DNA, 23 bp, Artificial Sequence
  primer
agcgtgcagataatgacaaggaa

SEQ ID NO: 1486 - DNA, 22 bp, Artificial Sequence
  primer
gatttgacggctcctctactgt

SEQ ID NO: 1487 - DNA, 21 bp, Artificial Sequence
  primer
cggtcttcatgcagagactga

SEQ ID NO: 1488 - DNA, 28 bp, Artificial Sequence
  primer
gtgaaatatagatgttccctccaggaat

SEQ ID NO: 1489 - DNA, 22 bp, Artificial Sequence
  primer
gacggattacaccttcccactt

SEQ ID NO: 1490 - DNA, 24 bp, Artificial Sequence
  primer
gactcttccttggcttcaacctta

SEQ ID NO: 1491 - DNA, 20 bp, Artificial Sequence
  primer
cgatgggctcagctttcaga

SEQ ID NO: 1492 - DNA, 21 bp, Artificial Sequence
  primer
acaaaacctcgtccacggaat

SEQ ID NO: 1493 - DNA, 20 bp, Artificial Sequence
  primer
tcctgcgtttggtggatgat

SEQ ID NO: 1494 - DNA, 24 bp, Artificial Sequence
  primer
cctcgtcttctacagggaagttca

SEQ ID NO: 1495 - DNA, 20 bp, Artificial Sequence
  primer
tgggtggtcctgcaaaatcc

SEQ ID NO: 1496 - DNA, 27 bp, Artificial Sequence
  primer
acatattgatttggagccagttcttca

SEQ ID NO: 1497 - DNA, 20 bp, Artificial Sequence
  primer
ctggcccctgtcatcttctg

SEQ ID NO: 1498 - DNA, 21 bp, Artificial Sequence
  primer
cttggccagttggcaaaacat

SEQ ID NO: 1499 - DNA, 19 bp, Artificial Sequence
  primer
ctggaactgtcccactgct

SEQ ID NO: 1500 - DNA, 23 bp, Artificial Sequence
  primer
caggattcgatggaaccttctga

SEQ ID NO: 1501 - DNA, 68 bp, Artificial Sequence
  amplicon
ggaaactcagcagatgttacagattctaggtggccttatttacataatagattggtggtgtttacatt

SEQ ID NO: 1502 - DNA, 78 bp, Artificial Sequence
  amplicon
ggaaactcagcagatgttacaggacgtggtccagattaagtttgcacgaggacgaggacaaggaaaacgatggagact

SEQ ID NO: 1503 - DNA, 87 bp, Artificial Sequence
  amplicon
ggaaactcagcagatgttacagcagcaacgaagtatcctctcctgaaattattaggcagcacttggccaaccaccccgccgcgaccc

SEQ ID NO: 1504 - DNA, 97 bp, Artificial Sequence
  amplicon
gatataatattagaatgtgtgtactgcaagcaacagttactgcgacgtgagattctaggtggccttatttacataatagattggtggtgtttacatt

SEQ ID NO: 1505 - DNA, 107 bp, Artificial Sequence
  amplicon
gatataatattagaatgtgtgtactgcaagcaacagttactgcgacgtgaggacgtggtccagattaagtttgcacgaggacgaggacaaggaaaacgatggagact

SEQ ID NO: 1506 - DNA, 116 bp, Artificial Sequence
  amplicon
gatataatattagaatgtgtgtactgcaagcaacagttactgcgacgtgagcagcaacgaagtatcctctcctgaaattattaggcagcacttggccaaccaccccgccgcgaccc

SEQ ID NO: 1507 - DNA, 81 bp, Artificial Sequence
  amplicon
gatataatattagaatgtgtgtactgcaagcaacagttactgcgacgtgaggtgtattaactgtcaaaagccactgtgtcc

SEQ ID NO: 1508 - DNA, 125 bp, Artificial Sequence
  amplicon
gatataatattagaatgtgtgtactgcaagcaacagttactgcgacgtgagatcatcaagaacacgtagagaaacccagctgtaatcatgcatggagatacacctacattgcatgaatatatgtt

SEQ ID NO: 1509 - DNA, 98 bp, Artificial Sequence
  amplicon
gatataatattagaatgtgtgtactgcaagcaacagttactgcgacgtgagtgtgactctacgcttcggttgtgcgtacaaagcacacacgtagacat

SEQ ID NO: 1510 - DNA, 86 bp, Artificial Sequence
  amplicon
tgtaatagtaacactacacccatagtacatttaaaagatgtctctttggctgcctagtgaggccactgtctacttgcctcctgtcc

SEQ ID NO: 1511 - DNA, 83 bp, Artificial Sequence
  amplicon
tctcagaaaccataatctaccatggctgatcctgcagattctaggtggccttatttacataatagattggtggtgtttacatt

SEQ ID NO: 1512 - DNA, 93 bp, Artificial Sequence
  amplicon
tctcagaaaccataatctaccatggctgatcctgcaggacgtggtccagattaagtttgcacgaggacgaggacaaggaaaacgatggagact

SEQ ID NO: 1513 - DNA, 102 bp, Artificial Sequence
  amplicon
tctcagaaaccataatctaccatggctgatcctgcagcagcaacgaagtatcctctcctgaaattattaggcagcacttggccaaccaccccgccgcgaccc

SEQ ID NO: 1514 - DNA, 86 bp, Artificial Sequence
  amplicon
tctcagaaaccataatctaccatggctgatcctgcagatgtctctttggctgcctagtgaggccactgtctacttgcctcctgtcc

SEQ ID NO: 1515 - DNA, 98 bp, Artificial Sequence
  amplicon
gaagtggaagcaacacagattcaggataatagatggccatatttagaaagtagaataacagtatttgaatttccaaatgcatttccatttgataaaaa

SEQ ID NO: 1516 - DNA, 62 bp, Artificial Sequence
  amplicon
gaagtggaagcaacacagattcaggacatggtccagattagatttgcacgaggaagaggaag

SEQ ID NO: 1517 - DNA, 88 bp, Artificial Sequence
  amplicon
gaagtggaagcaacacagattcagcttgttaaacagctacagcacaccccctcaccgtattccagcaccgtgtccgtgggcaccgcaa

SEQ ID NO: 1518 - DNA, 107 bp, Artificial Sequence
  amplicon
gtatattgcaagacagtattggaacttacagaggataatagatggccatatttagaaagtagaataacagtatttgaatttccaaatgcatttccatttgataaaaa

SEQ ID NO: 1519 - DNA, 71 bp, Artificial Sequence
  amplicon
gtatattgcaagacagtattggaacttacagaggacatggtccagattagatttgcacgaggaagaggaag

SEQ ID NO: 1520 - DNA, 97 bp, Artificial Sequence
  amplicon
gtatattgcaagacagtattggaacttacagagcttgttaaacagctacagcacaccccctcaccgtattccagcaccgtgtccgtgggcaccgcaa

SEQ ID NO: 1521 - DNA, 96 bp, Artificial Sequence
  amplicon
gtatattgcaagacagtattggaacttacagaggtgcctgcggtgccagaaaccgttgaatccagcagaaaaacttagacaccttaatgaaaaacg

SEQ ID NO: 1522 - DNA, 83 bp, Artificial Sequence
  amplicon
gtatattgcaagacagtattggaacttacagagtgtgaagccagaattgagctagtagtagaaagctcagcagacgaccttcg

SEQ ID NO: 1523 - DNA, 110 bp, Artificial Sequence
  amplicon
tgcgaggaactatggaatacagaacctactcactgctttaaaaaagcttgttaaacagctacagcacaccccctcaccgtattccagcaccgtgtccgtgggcaccgcaa

SEQ ID NO: 1524 - DNA, 97 bp, Artificial Sequence
  amplicon
caaaagacggaaactctgtagtggtaacactacgcctataatacatttaaaagatggctttgtggcggcctagtgacaataccgtatatcttccacc

SEQ ID NO: 1525 - DNA, 98 bp, Artificial Sequence
  amplicon
caaaagacggaaactctgtagtggtaacactacgcctataatacatttaaaaggtggtggcaataagcaggatattcctaaggtttctgcataccaat

SEQ ID NO: 1526 - DNA, 69 bp, Artificial Sequence
  amplicon
atatcatccacctggcattggacagatggctttgtggcggcctagtgacaataccgtatatcttccacc

SEQ ID NO: 1527 - DNA, 70 bp, Artificial Sequence
  amplicon
atatcatccacctggcattggacaggtggtggcaataagcaggatattcctaaggtttctgcataccaat

SEQ ID NO: 1528 - DNA, 92 bp, Artificial Sequence
  amplicon
caatggctgatccagaaggataatagatggccatatttagaaagtagaataacagtatttgaatttccaaatgcatttccatttgataaaaa

SEQ ID NO: 1529 - DNA, 56 bp, Artificial Sequence
  amplicon
caatggctgatccagaaggacatggtccagattagatttgcacgaggaagaggaag

SEQ ID NO: 1530 - DNA, 82 bp, Artificial Sequence
  amplicon
caatggctgatccagaagcttgttaaacagctacagcacaccccctcaccgtattccagcaccgtgtccgtgggcaccgcaa

SEQ ID NO: 1531 - DNA, 62 bp, Artificial Sequence
  amplicon
caatggctgatccagaagatggctttgtggcggcctagtgacaataccgtatatcttccacc

SEQ ID NO: 1532 - DNA, 63 bp, Artificial Sequence
  amplicon
caatggctgatccagaaggtggtggcaataagcaggatattcctaaggtttctgcataccaat

SEQ ID NO: 1533 - DNA, 41 bp, Artificial Sequence
  amplicon
ggaaacgcagcagatggtacaggatgacagatggccatacc

SEQ ID NO: 1534 - DNA, 63 bp, Artificial Sequence
  amplicon
ggaaacgcagcagatggtacaggacgtggtgcagattaaatttgcacgaggaagaggacaaag

SEQ ID NO: 1535 - DNA, 70 bp, Artificial Sequence
  amplicon
ggaaacgcagcagatggtacagcagtgacgaaatatcctttgctgggattgttacaaagctaccaacagc

SEQ ID NO: 1536 - DNA, 70 bp, Artificial Sequence
  amplicon
gaactaagattgaattgtgtctactgcaaaggtcagttaacagaaacagaggatgacagatggccatacc

SEQ ID NO: 1537 - DNA, 92 bp, Artificial Sequence
  amplicon
gaactaagattgaattgtgtctactgcaaaggtcagttaacagaaacagaggacgtggtgcagattaaatttgcacgaggaagaggacaaag

SEQ ID NO: 1538 - DNA, 99 bp, Artificial Sequence
  amplicon
gaactaagattgaattgtgtctactgcaaaggtcagttaacagaaacagagcagtgacgaaatatcctttgctgggattgttacaaagctaccaacagc

SEQ ID NO: 1539 - DNA, 66 bp, Artificial Sequence
  amplicon
gaactaagattgaattgtgtctactgcaaaggtcagttaacagaaacagaggtgtataacgtgtca

SEQ ID NO: 1540 - DNA, 91 bp, Artificial Sequence
  amplicon
gaactaagattgaattgtgtctactgcaaaggtcagttaacagaaacagagaagacctcgtactgaaacccaagtgtaaacatgcgtggag

SEQ ID NO: 1541 - DNA, 95 bp, Artificial Sequence
  amplicon
gaactaagattgaattgtgtctactgcaaaggtcagttaacagaaacagagtgtaagtctacacttcgtttgtgtgtacagagcacacaagtaga

SEQ ID NO: 1542 - DNA, 87 bp, Artificial Sequence
  amplicon
aacaagggctgtcagttgtcctgcaactacacctataatacacttaaaagatgtctctgtggcggcctagcgaggctactgtctact

SEQ ID NO: 1543 - DNA, 55 bp, Artificial Sequence
  amplicon
tctactagactgtaactacaatggctgatccagcaggatgacagatggccatacc

SEQ ID NO: 1544 - DNA, 77 bp, Artificial Sequence
  amplicon
tctactagactgtaactacaatggctgatccagcaggacgtggtgcagattaaatttgcacgaggaagaggacaaag

SEQ ID NO: 1545 - DNA, 84 bp, Artificial Sequence
  amplicon
tctactagactgtaactacaatggctgatccagcagcagtgacgaaatatcctttgctgggattgttacaaagctaccaacagc

SEQ ID NO: 1546 - DNA, 73 bp, Artificial Sequence
  amplicon
tctactagactgtaactacaatggctgatccagcagatgtctctgtggcggcctagcgaggctactgtctact

SEQ ID NO: 1547 - DNA, 100 bp, Artificial Sequence
  amplicon
gcaatactgaagtggaaactcagcagatggtacaacagactctagatggccatatttacatagtagattaacagtatttgaatttaaaaatccattccca

SEQ ID NO: 1548 - DNA, 72 bp, Artificial Sequence
  amplicon
gcaatactgaagtggaaactcagcagatggtacaacaggacgtggtgcaaattagatttaatagaggaagag

SEQ ID NO: 1549 - DNA, 79 bp, Artificial Sequence
  amplicon
gcaatactgaagtggaaactcagcagatggtacaacagcaaccaaatatccactactgaaactgctgacatacagacag

SEQ ID NO: 1550 - DNA, 107 bp, Artificial Sequence
  amplicon
gaactacagtgcgtggaatgcaaaaaacctttgcaacgatctgagactctagatggccatatttacatagtagattaacagtatttgaatttaaaaatccattccca

SEQ ID NO: 1551 - DNA, 79 bp, Artificial Sequence
  amplicon
gaactacagtgcgtggaatgcaaaaaacctttgcaacgatctgaggacgtggtgcaaattagatttaatagaggaagag

SEQ ID NO: 1552 - DNA, 86 bp, Artificial Sequence
  amplicon
gaactacagtgcgtggaatgcaaaaaacctttgcaacgatctgagcaaccaaatatccactactgaaactgctgacatacagacag

SEQ ID NO: 1553 - DNA, 84 bp, Artificial Sequence
  amplicon
gaactacagtgcgtggaatgcaaaaaacctttgcaacgatctgaggtgtattatatgtcaaagacctttgtgtcctcaagaaaa

SEQ ID NO: 1554 - DNA, 95 bp, Artificial Sequence
  amplicon
gaactacagtgcgtggaatgcaaaaaacctttgcaacgatctgaggtcccgacgtagagaaactgcactgtgacgtgtaaaaacgccatgagagg

SEQ ID NO: 1555 - DNA, 102 bp, Artificial Sequence
  amplicon
acaagcagcggactgtgtgtagttctaacgttgcacctatagtgcatttaaaagatgtccgtgtggcggcctagtgaggccacagtgtacctgcctcctgta

SEQ ID NO: 1556 - DNA, 96 bp, Artificial Sequence
  amplicon
caataaacatcatctacaatggccgatcctgaagactctagatggccatatttacatagtagattaacagtatttgaatttaaaaatccattccca

SEQ ID NO: 1557 - DNA, 68 bp, Artificial Sequence
  amplicon
caataaacatcatctacaatggccgatcctgaaggacgtggtgcaaattagatttaatagaggaagag

SEQ ID NO: 1558 - DNA, 75 bp, Artificial Sequence
  amplicon
caataaacatcatctacaatggccgatcctgaagcaaccaaatatccactactgaaactgctgacatacagacag

SEQ ID NO: 1559 - DNA, 82 bp, Artificial Sequence
  amplicon
caataaacatcatctacaatggccgatcctgaagatgtccgtgtggcggcctagtgaggccacagtgtacctgcctcctgta

SEQ ID NO: 1560 - DNA, 65 bp, Artificial Sequence
  amplicon
atggcaattctgaagtggaaatacagcagatacaacagatgacaggtggccatacttacatagca

SEQ ID NO: 1561 - DNA, 74 bp, Artificial Sequence
  amplicon
atggcaattctgaagtggaaatacagcagatacaacaggacgtggtgcagattaaatttgcacgaggaagagga

SEQ ID NO: 1562 - DNA, 93 bp, Artificial Sequence
  amplicon
atggcaattctgaagtggaaatacagcagatacaacagcagcacagaactatccactgctgaaattgctacacagctacacgcctacaacacc

SEQ ID NO: 1563 - DNA, 73 bp, Artificial Sequence
  amplicon
ttgtttgaattgtgtatactgcaaacaagaattacagcggagtgagatgacaggtggccatacttacatagca

SEQ ID NO: 1564 - DNA, 82 bp, Artificial Sequence
  amplicon
ttgtttgaattgtgtatactgcaaacaagaattacagcggagtgaggacgtggtgcagattaaatttgcacgaggaagagga

SEQ ID NO: 1565 - DNA, 101 bp, Artificial Sequence
  amplicon
ttgtttgaattgtgtatactgcaaacaagaattacagcggagtgagcagcacagaactatccactgctgaaattgctacacagctacacgcctacaacacc

SEQ ID NO: 1566 - DNA, 106 bp, Artificial Sequence
  amplicon
ttgtttgaattgtgtatactgcaaacaagaattacagcggagtgaggtgtattacatgtcaaaaaccgctgtgtccagttgaaaagcaaagacatttagaagaaaa

SEQ ID NO: 1567 - DNA, 97 bp, Artificial Sequence
  amplicon
ccggtgtggtagttgtagtacaactacacctatagtacatttaaaagatgtctctgtggcggtctaacgaagccactgtctacctgcctccagtgtc

SEQ ID NO: 1568 - DNA, 69 bp, Artificial Sequence
  amplicon
ccggtgtggtagttgtagtacaactacacctatagtacatttaaaagattctaataaaatagcagtacc

SEQ ID NO: 1569 - DNA, 48 bp, Artificial Sequence
  amplicon
ctacaatggctgatcctgcagatgacaggtggccatacttacatagca

SEQ ID NO: 1570 - DNA, 57 bp, Artificial Sequence
  amplicon
ctacaatggctgatcctgcaggacgtggtgcagattaaatttgcacgaggaagagga

SEQ ID NO: 1571 - DNA, 76 bp, Artificial Sequence
  amplicon
ctacaatggctgatcctgcagcagcacagaactatccactgctgaaattgctacacagctacacgcctacaacacc

SEQ ID NO: 1572 - DNA, 71 bp, Artificial Sequence
  amplicon
ctacaatggctgatcctgcagatgtctctgtggcggtctaacgaagccactgtctacctgcctccagtgtc

SEQ ID NO: 1573 - DNA, 43 bp, Artificial Sequence
  amplicon
ctacaatggctgatcctgcagattctaataaaatagcagtacc

SEQ ID NO: 1574 - DNA, 92 bp, Artificial Sequence
  amplicon
gcggatatggcaatatggaagtggaaacagctgaagtggaggagacgataggtggccatatttacgtagtaggctaacagtgtttaaatttc

SEQ ID NO: 1575 - DNA, 81 bp, Artificial Sequence
  amplicon
gcggatatggcaatatggaagtggaaacagctgaagtggaggagtgacggatcggtacccactactgaacttactaccgaa

SEQ ID NO: 1576 - DNA, 87 bp, Artificial Sequence
  amplicon
gcctgtgtctattgcagacgaccactacagcaaaccgagacgataggtggccatatttacgtagtaggctaacagtgtttaaatttc

SEQ ID NO: 1577 - DNA, 76 bp, Artificial Sequence
  amplicon
gcctgtgtctattgcagacgaccactacagcaaaccgagtgacggatcggtacccactactgaacttactaccgaa

SEQ ID NO: 1578 - DNA, 112 bp, Artificial Sequence
  amplicon
gcctgtgtctattgcagacgaccactacagcaaaccgaggtgcatgtgttgtctgaaaccgctgtgtccagcagaaaaattaagacacctaaatagcaaacgaagatttcat

SEQ ID NO: 1579 - DNA, 78 bp, Artificial Sequence
  amplicon
gcctgtgtctattgcagacgaccactacagcaaaccgagtgtaacaacacactgcagctggtagtagaagcctcacgg

SEQ ID NO: 1580 - DNA, 104 bp, Artificial Sequence
  amplicon
acacaagacggtacctcagttgtggtaacactacgcctataatacatttaaaagatggctatgtggcggtctagtgacagcatggtgtatttgcctccaccttc

SEQ ID NO: 1581 - DNA, 78 bp, Artificial Sequence
  amplicon
accagtaacctgctatggccaatcgtgaagacgataggtggccatatttacgtagtaggctaacagtgtttaaatttc

SEQ ID NO: 1582 - DNA, 67 bp, Artificial Sequence
  amplicon
accagtaacctgctatggccaatcgtgaagtgacggatcggtacccactactgaacttactaccgaa

SEQ ID NO: 1583 - DNA, 80 bp, Artificial Sequence
  amplicon
accagtaacctgctatggccaatcgtgaagatggctatgtggcggtctagtgacagcatggtgtatttgcctccaccttc

SEQ ID NO: 1584 - DNA, 56 bp, Artificial Sequence
  amplicon
gaagtggaagctgcagagactcagataataaatggccatatttagaaagtagggtg

SEQ ID NO: 1585 - DNA, 62 bp, Artificial Sequence
  amplicon
gaagtggaagctgcagagactcaggacatggtccagattagatttgcacgaggacgatgaag

SEQ ID NO: 1586 - DNA, 62 bp, Artificial Sequence
  amplicon
gaagtggaagctgcagagactcagattgttagacagctacaacacgcctccacgtcgacccc

SEQ ID NO: 1587 - DNA, 63 bp, Artificial Sequence
  amplicon
atattgcaaagcaacattggaacgcacagagataataaatggccatatttagaaagtagggtg

SEQ ID NO: 1588 - DNA, 69 bp, Artificial Sequence
  amplicon
atattgcaaagcaacattggaacgcacagaggacatggtccagattagatttgcacgaggacgatgaag

SEQ ID NO: 1589 - DNA, 69 bp, Artificial Sequence
  amplicon
atattgcaaagcaacattggaacgcacagagattgttagacagctacaacacgcctccacgtcgacccc

SEQ ID NO: 1590 - DNA, 67 bp, Artificial Sequence
  amplicon
atattgcaaagcaacattggaacgcacagaggtgcctgcggtgccagaaaccattgaacccagcaga

SEQ ID NO: 1591 - DNA, 79 bp, Artificial Sequence
  amplicon
atattgcaaagcaacattggaacgcacagagtgtgacggcagaattgagcttacagtagagagctcggcagaggacctt

SEQ ID NO: 1592 - DNA, 100 bp, Artificial Sequence
  amplicon
aagaaggaaagtgtgtagtggtaacactacgcctataatacacttaaaagatggctttgtggcggcctagtgacagtacggtatatcttccaccaccttc

SEQ ID NO: 1593 - DNA, 75 bp, Artificial Sequence
  amplicon
atatcctccacctggcattggacagatggctttgtggcggcctagtgacagtacggtatatcttccaccaccttc

SEQ ID NO: 1594 - DNA, 78 bp, Artificial Sequence
  amplicon
ccgtggtgtgcaactaaccaataatctacaatggcggatccagaagataataaatggccatatttagaaagtagggtg

SEQ ID NO: 1595 - DNA, 84 bp, Artificial Sequence
  amplicon
ccgtggtgtgcaactaaccaataatctacaatggcggatccagaaggacatggtccagattagatttgcacgaggacgatgaag

SEQ ID NO: 1596 - DNA, 84 bp, Artificial Sequence
  amplicon
ccgtggtgtgcaactaaccaataatctacaatggcggatccagaagattgttagacagctacaacacgcctccacgtcgacccc

SEQ ID NO: 1597 - DNA, 96 bp, Artificial Sequence
  amplicon
ccgtggtgtgcaactaaccaataatctacaatggcggatccagaagatggctttgtggcggcctagtgacagtacggtatatcttccaccaccttc

SEQ ID NO: 1598 - DNA, 105 bp, Artificial Sequence
  amplicon
cacaagtggaaactgtggaagcaacgttgcaggatgcaaacctaatgtatttacatacaagggtaacagtattaaagtttttaaatacatttccatttgataaca

SEQ ID NO: 1599 - DNA, 100 bp, Artificial Sequence
  amplicon
cacaagtggaaactgtggaagcaacgttgcagtacctgcagcgacgcgttatccactactacaactgttgaacaactatcaaacaccccaacgaccaatc

SEQ ID NO: 1600 - DNA, 134 bp, Artificial Sequence
  amplicon
gtttctatgcacaatatacaggtagtgtgtgtgtattgtaaaaaggaattatgtagagcaggatgcaaacctaatgtatttacatacaagggtaacagtattaaagtttttaaatacatttccatttgataaca

SEQ ID NO: 1601 - DNA, 129 bp, Artificial Sequence
  amplicon
gtttctatgcacaatatacaggtagtgtgtgtgtattgtaaaaaggaattatgtagagcagtacctgcagcgacgcgttatccactactacaactgttgaacaactatcaaacaccccaacgaccaatc

SEQ ID NO: 1602 - DNA, 122 bp, Artificial Sequence
  amplicon
gtttctatgcacaatatacaggtagtgtgtgtgtattgtaaaaaggaattatgtagagcaggtgtcatagatgtcaaagaccacttgggcctgaagaaaagcaaaaattggtggacgaaaaa

SEQ ID NO: 1603 - DNA, 107 bp, Artificial Sequence
  amplicon
gtttctatgcacaatatacaggtagtgtgtgtgtattgtaaaaaggaattatgtagagcaggtgttcaagtgtagtacaactggcagtggaaagcagtggagacacc

SEQ ID NO: 1604 - DNA, 79 bp, Artificial Sequence
  amplicon
gtgcaactcagactgcgtttatagtgcatttaaaagatggcattgtggcgcactaatgacagcaaggtgtatttgccac

SEQ ID NO: 1605 - DNA, 117 bp, Artificial Sequence
  amplicon
gcccgtgttgtgcgaacaactagcaacggcgatggactgtgaaggatgcaaacctaatgtatttacatacaagggtaacagtattaaagtttttaaatacatttccatttgataaca

SEQ ID NO: 1606 - DNA, 112 bp, Artificial Sequence
  amplicon
gcccgtgttgtgcgaacaactagcaacggcgatggactgtgaagtacctgcagcgacgcgttatccactactacaactgttgaacaactatcaaacaccccaacgaccaatc

SEQ ID NO: 1607 - DNA, 87 bp, Artificial Sequence
  amplicon
gcccgtgttgtgcgaacaactagcaacggcgatggactgtgaagatggcattgtggcgcactaatgacagcaaggtgtatttgccac

SEQ ID NO: 1608 - DNA, 73 bp, Artificial Sequence
  amplicon
cggctatggcaatagtgaagtggaagcgcagcagatggcagaccagatcctaggtggccatatttacatagta

SEQ ID NO: 1609 - DNA, 85 bp, Artificial Sequence
  amplicon
cggctatggcaatagtgaagtggaagcgcagcagatggcagaccaggacgtggtgcaaattagatttaatacaggaagaggacaa

SEQ ID NO: 1610 - DNA, 90 bp, Artificial Sequence
  amplicon
cggctatggcaatagtgaagtggaagcgcagcagatggcagaccagtaacgaagtatccactactgaaactgctgtccacctatgcaccg

SEQ ID NO: 1611 - DNA, 67 bp, Artificial Sequence
  amplicon
gcagtgtgtgcagtgcaaaaaagagctacaacgaagagagatcctaggtggccatatttacatagta

SEQ ID NO: 1612 - DNA, 79 bp, Artificial Sequence
  amplicon
gcagtgtgtgcagtgcaaaaaagagctacaacgaagagaggacgtggtgcaaattagatttaatacaggaagaggacaa

SEQ ID NO: 1613 - DNA, 84 bp, Artificial Sequence
  amplicon
gcagtgtgtgcagtgcaaaaaagagctacaacgaagagagtaacgaagtatccactactgaaactgctgtccacctatgcaccg

SEQ ID NO: 1614 - DNA, 79 bp, Artificial Sequence
  amplicon
gcagtgtgtgcagtgcaaaaaagagctacaacgaagagagatgtataatttgtcaaacgccattatgtcctgaagaaaa

SEQ ID NO: 1615 - DNA, 103 bp, Artificial Sequence
  amplicon
gcagtgtgtgcagtgcaaaaaagagctacaacgaagagagaccccgacctgtgacccaagtgtaacgtcatgcgtggagacaaagcaactataaaagattata

SEQ ID NO: 1616 - DNA, 84 bp, Artificial Sequence
  amplicon
gcagtgtgtgcagtgcaaaaaagagctacaacgaagagagttgtgatagcacactacggctatgcattcatagcactgcgacgg

SEQ ID NO: 1617 - DNA, 98 bp, Artificial Sequence
  amplicon
acaaaggacgggttgcacatacaacttgtactgcacctataatacacctaaaagatgtccgtgtggcggcctagtgaggccactgtgtacctgcctcc

SEQ ID NO: 1618 - DNA, 89 bp, Artificial Sequence
  amplicon
acaaaggacgggttgcacatacaacttgtactgcacctataatacacctaaaagtagtggtaatggtaaaaaagttttagttcccaagg

SEQ ID NO: 1619 - DNA, 81 bp, Artificial Sequence
  amplicon
gtgtgccccggctgtgcacggctataaacaaccctgcaatggaggaccctgaagatcctaggtggccatatttacatagta

SEQ ID NO: 1620 - DNA, 93 bp, Artificial Sequence
  amplicon
gtgtgccccggctgtgcacggctataaacaaccctgcaatggaggaccctgaaggacgtggtgcaaattagatttaatacaggaagaggacaa

SEQ ID NO: 1621 - DNA, 98 bp, Artificial Sequence
  amplicon
gtgtgccccggctgtgcacggctataaacaaccctgcaatggaggaccctgaagtaacgaagtatccactactgaaactgctgtccacctatgcaccg

SEQ ID NO: 1622 - DNA, 98 bp, Artificial Sequence
  amplicon
gtgtgccccggctgtgcacggctataaacaaccctgcaatggaggaccctgaagatgtccgtgtggcggcctagtgaggccactgtgtacctgcctcc

SEQ ID NO: 1623 - DNA, 89 bp, Artificial Sequence
  amplicon
gtgtgccccggctgtgcacggctataaacaaccctgcaatggaggaccctgaagtagtggtaatggtaaaaaagttttagttcccaagg

SEQ ID NO: 1624 - DNA, 48 bp, Artificial Sequence
  amplicon
cattggaaactctggaaacaccagaacagatgctaaattacgatattt

SEQ ID NO: 1625 - DNA, 48 bp, Artificial Sequence
  amplicon
cattggaaactctggaaacaccagaacaggacgtggtccagattaaat

SEQ ID NO: 1626 - DNA, 84 bp, Artificial Sequence
  amplicon
cattggaaactctggaaacaccagaacagtacctgtagatacaacgtatcccctgttgaaactgttaacgaatacaacacccac

SEQ ID NO: 1627 - DNA, 82 bp, Artificial Sequence
  amplicon
atacctttaattgatcttagattatcatgtgtatattgcaaaaaagaactaacacgtgctgagatgctaaattacgatattt

SEQ ID NO: 1628 - DNA, 82 bp, Artificial Sequence
  amplicon
atacctttaattgatcttagattatcatgtgtatattgcaaaaaagaactaacacgtgctgaggacgtggtccagattaaat

SEQ ID NO: 1629 - DNA, 118 bp, Artificial Sequence
  amplicon
atacctttaattgatcttagattatcatgtgtatattgcaaaaaagaactaacacgtgctgagtacctgtagatacaacgtatcccctgttgaaactgttaacgaatacaacacccac

SEQ ID NO: 1630 - DNA, 85 bp, Artificial Sequence
  amplicon
atacctttaattgatcttagattatcatgtgtatattgcaaaaaagaactaacacgtgctgaggtgctacagatgtcaaagtccg

SEQ ID NO: 1631 - DNA, 115 bp, Artificial Sequence
  amplicon
atacctttaattgatcttagattatcatgtgtatattgcaaaaaagaactaacacgtgctgagacaaacatctagagaacctagagaatctacagtataatcatgcatggtaaag

SEQ ID NO: 1632 - DNA, 102 bp, Artificial Sequence
  amplicon
atacctttaattgatcttagattatcatgtgtatattgcaaaaaagaactaacacgtgctgagtgtaagtttgtggtgcagttggacattcagagtaccaaa

SEQ ID NO: 1633 - DNA, 66 bp, Artificial Sequence
  amplicon
actacgcctgtagtacatttaaaagatggcgacgtggcggcctagtgaaaataaggtgtatctacc

SEQ ID NO: 1634 - DNA, 63 bp, Artificial Sequence
  amplicon
actacgcctgtagtacatttaaaaggacaataccaaaacaaacattcccaaagttagtgcata

SEQ ID NO: 1635 - DNA, 57 bp, Artificial Sequence
  amplicon
gcgcatcaagtaactaactgcaatggcgtcacctgaagatgctaaattacgatattt

SEQ ID NO: 1636 - DNA, 57 bp, Artificial Sequence
  amplicon
gcgcatcaagtaactaactgcaatggcgtcacctgaaggacgtggtccagattaaat

SEQ ID NO: 1637 - DNA, 93 bp, Artificial Sequence
  amplicon
gcgcatcaagtaactaactgcaatggcgtcacctgaagtacctgtagatacaacgtatcccctgttgaaactgttaacgaatacaacacccac

SEQ ID NO: 1638 - DNA, 79 bp, Artificial Sequence
  amplicon
gcgcatcaagtaactaactgcaatggcgtcacctgaagatggcgacgtggcggcctagtgaaaataaggtgtatctacc

SEQ ID NO: 1639 - DNA, 76 bp, Artificial Sequence
  amplicon
gcgcatcaagtaactaactgcaatggcgtcacctgaaggacaataccaaaacaaacattcccaaagttagtgcata

SEQ ID NO: 1640 - DNA, 81 bp, Artificial Sequence
  amplicon
atggcaatactgaagtggaaactgagcagatggcacaccagattcacgatggccatatttgcacagtagactaacagtatt

SEQ ID NO: 1641 - DNA, 75 bp, Artificial Sequence
  amplicon
atggcaatactgaagtggaaactgagcagatggcacaccaggacgtggtgcaaattaggcttaatagaggaagag

SEQ ID NO: 1642 - DNA, 90 bp, Artificial Sequence
  amplicon
atggcaatactgaagtggaaactgagcagatggcacaccagtgatcaaatatccactactgaaactgctgacccaaagaccaccgaggcc

SEQ ID NO: 1643 - DNA, 97 bp, Artificial Sequence
  amplicon
gtgcatgaaatcgaattgaaatgcgttgaatgcaaaaagactttgcagcgatctgagattcacgatggccatatttgcacagtagactaacagtatt

SEQ ID NO: 1644 - DNA, 91 bp, Artificial Sequence
  amplicon
gtgcatgaaatcgaattgaaatgcgttgaatgcaaaaagactttgcagcgatctgaggacgtggtgcaaattaggcttaatagaggaagag

SEQ ID NO: 1645 - DNA, 106 bp, Artificial Sequence
  amplicon
gtgcatgaaatcgaattgaaatgcgttgaatgcaaaaagactttgcagcgatctgagtgatcaaatatccactactgaaactgctgacccaaagaccaccgaggcc

SEQ ID NO: 1646 - DNA, 116 bp, Artificial Sequence
  amplicon
gtgcatgaaatcgaattgaaatgcgttgaatgcaaaaagactttgcagcgatctgagatgtattatttgtcaaagaccattgtgtccacaagaaaaaaaaaggcatgtggatttaa

SEQ ID NO: 1647 - DNA, 100 bp, Artificial Sequence
  amplicon
gtgcatgaaatcgaattgaaatgcgttgaatgcaaaaagactttgcagcgatctgagaccccgacgtagacaaacacaagtgtaacctgtaacaacgcca

SEQ ID NO: 1648 - DNA, 106 bp, Artificial Sequence
  amplicon
gtacatacaaagggcggaacgtgtgtagttctaaagtttcacctatcgtgcatttaaaagatgtccgtgtggcggcctagtgaggccactgtgtacctgcctcctg

SEQ ID NO: 1649 - DNA, 95 bp, Artificial Sequence
  amplicon
gtgtgccctagctgtgcacagcaataaacaccatctgcaatggatgaccctgaagattcacgatggccatatttgcacagtagactaacagtatt

SEQ ID NO: 1650 - DNA, 89 bp, Artificial Sequence
  amplicon
gtgtgccctagctgtgcacagcaataaacaccatctgcaatggatgaccctgaaggacgtggtgcaaattaggcttaatagaggaagag

SEQ ID NO: 1651 - DNA, 104 bp, Artificial Sequence
  amplicon
gtgtgccctagctgtgcacagcaataaacaccatctgcaatggatgaccctgaagtgatcaaatatccactactgaaactgctgacccaaagaccaccgaggcc

SEQ ID NO: 1652 - DNA, 101 bp, Artificial Sequence
  amplicon
gtgtgccctagctgtgcacagcaataaacaccatctgcaatggatgaccctgaagatgtccgtgtggcggcctagtgaggccactgtgtacctgcctcctg

SEQ ID NO: 1653 - DNA, 87 bp, Artificial Sequence
  amplicon
gcggctatggctattctgaagtggaaatgctcgagactcagataacaggtggccatatttaaatagcagattaatggtatttaaatt

SEQ ID NO: 1654 - DNA, 79 bp, Artificial Sequence
  amplicon
gcggctatggctattctgaagtggaaatgctcgagactcaggacgtggtgcagattagatttgaacgaggaagaggaag

SEQ ID NO: 1655 - DNA, 103 bp, Artificial Sequence
  amplicon
gcggctatggctattctgaagtggaaatgctcgagactcagtgacgagcaagtatccactgctggatcttctgagcaactatcatacccctccgcaacgcccc

SEQ ID NO: 1656 - DNA, 65 bp, Artificial Sequence
  amplicon
ggaactgcaagaaagagagataacaggtggccatatttaaatagcagattaatggtatttaaatt

SEQ ID NO: 1657 - DNA, 57 bp, Artificial Sequence
  amplicon
ggaactgcaagaaagagaggacgtggtgcagattagatttgaacgaggaagaggaag

SEQ ID NO: 1658 - DNA, 81 bp, Artificial Sequence
  amplicon
ggaactgcaagaaagagagtgacgagcaagtatccactgctggatcttctgagcaactatcatacccctccgcaacgcccc

SEQ ID NO: 1659 - DNA, 59 bp, Artificial Sequence
  amplicon
ggaactgcaagaaagagagtgtaataatcaacttcagctagtagtagaaacctcgcaag

SEQ ID NO: 1660 - DNA, 116 bp, Artificial Sequence
  amplicon
caacccgcgacggcacatcccttgcagtaacactacgcctataatacacttaaaagatggctctatggcgttctagtgacaacaaggtgtatctacctccaccttcggtagctaag

SEQ ID NO: 1661 - DNA, 118 bp, Artificial Sequence
  amplicon
caacccgcgacggcacatcccttgcagtaacactacgcctataatacacttaaaaggtggtaatggtagacaggatgttcctaaggtgtctgcatatcaatacagagtatttagggtt

SEQ ID NO: 1662 - DNA, 83 bp, Artificial Sequence
  amplicon
gcagcaaaccagtaacctgcaatggccgattcggaagataacaggtggccatatttaaatagcagattaatggtatttaaatt

SEQ ID NO: 1663 - DNA, 75 bp, Artificial Sequence
  amplicon
gcagcaaaccagtaacctgcaatggccgattcggaaggacgtggtgcagattagatttgaacgaggaagaggaag

SEQ ID NO: 1664 - DNA, 99 bp, Artificial Sequence
  amplicon
gcagcaaaccagtaacctgcaatggccgattcggaagtgacgagcaagtatccactgctggatcttctgagcaactatcatacccctccgcaacgcccc

SEQ ID NO: 1665 - DNA, 97 bp, Artificial Sequence
  amplicon
gcagcaaaccagtaacctgcaatggccgattcggaagatggctctatggcgttctagtgacaacaaggtgtatctacctccaccttcggtagctaag

SEQ ID NO: 1666 - DNA, 99 bp, Artificial Sequence
  amplicon
gcagcaaaccagtaacctgcaatggccgattcggaaggtggtaatggtagacaggatgttcctaaggtgtctgcatatcaatacagagtatttagggtt

SEQ ID NO: 1667 - DNA, 100 bp, Artificial Sequence
  amplicon
cattggaaacattggaaacatcacaacagatgcaaaattaagatatttacacagtagaatttcagtgtttaagtttgaaaatccatttccattagataac

SEQ ID NO: 1668 - DNA, 48 bp, Artificial Sequence
  amplicon
cattggaaacattggaaacatcacaacaggacatggtccagattaaat

SEQ ID NO: 1669 - DNA, 84 bp, Artificial Sequence
  amplicon
gcaagtacaaacagcacatgcagatgcacagacgttgcaaaaactaaaacgaaagtatataggtagtcccttaagtgatattag

SEQ ID NO: 1670 - DNA, 50 bp, Artificial Sequence
  amplicon
cattggaaacattggaaacatcacaacagactgttaacgaatacaacaac

SEQ ID NO: 1671 - DNA, 135 bp, Artificial Sequence
  amplicon
aatacctttacttgatcttagattatcatgtgtatactgcaaaaaggaacttacaagtttagagatgcaaaattaagatatttacacagtagaatttcagtgtttaagtttgaaaatccatttccattagataac

SEQ ID NO: 1672 - DNA, 83 bp, Artificial Sequence
  amplicon
aatacctttacttgatcttagattatcatgtgtatactgcaaaaaggaacttacaagtttagaggacatggtccagattaaat

SEQ ID NO: 1673 - DNA, 85 bp, Artificial Sequence
  amplicon
aatacctttacttgatcttagattatcatgtgtatactgcaaaaaggaacttacaagtttagagactgttaacgaatacaacaac

SEQ ID NO: 1674 - DNA, 107 bp, Artificial Sequence
  amplicon
aatacctttacttgatcttagattatcatgtgtatactgcaaaaaggaacttacaagtttagaggtgctaccgatgtcaatgtccgttaacaccggaggaaaaacaa

SEQ ID NO: 1675 - DNA, 114 bp, Artificial Sequence
  amplicon
aatacctttacttgatcttagattatcatgtgtatactgcaaaaaggaacttacaagtttagagacatacgagtagacaagctacagaatctacagtataaccatgcatggtaa

SEQ ID NO: 1676 - DNA, 102 bp, Artificial Sequence
  amplicon
aatacctttacttgatcttagattatcatgtgtatactgcaaaaaggaacttacaagtttagagtgtgagttggtggtgcagttggacattcagagtaccaa

SEQ ID NO: 1677 - DNA, 75 bp, Artificial Sequence
  amplicon
ggtgataaaactacgcctgtaatccatttaaaagatggcgatgtggcggcctagtgacaataaggtgtacctacc

SEQ ID NO: 1678 - DNA, 109 bp, Artificial Sequence
  amplicon
gcgcatcatctaaataactgcaatggcatcacctgaagatgcaaaattaagatatttacacagtagaatttcagtgtttaagtttgaaaatccatttccattagataac

SEQ ID NO: 1679 - DNA, 57 bp, Artificial Sequence
  amplicon
gcgcatcatctaaataactgcaatggcatcacctgaaggacatggtccagattaaat

SEQ ID NO: 1680 - DNA, 59 bp, Artificial Sequence
  amplicon
gcgcatcatctaaataactgcaatggcatcacctgaagactgttaacgaatacaacaac

SEQ ID NO: 1681 - DNA, 79 bp, Artificial Sequence
  amplicon
gcgcatcatctaaataactgcaatggcatcacctgaagatggcgatgtggcggcctagtgacaataaggtgtacctacc

SEQ ID NO: 1682 - DNA, 93 bp, Artificial Sequence
  amplicon
ccggacagcggctatggcaatatggaagtggaaactaactcggagacaataggtggccgtatttacatagtagactaaccgtgtttaaatttc

SEQ ID NO: 1683 - DNA, 88 bp, Artificial Sequence
  amplicon
ccggacagcggctatggcaatatggaagtggaaactaactcggagtaccactgacggaaaagtatccactactgaatctgttgccgac

SEQ ID NO: 1684 - DNA, 96 bp, Artificial Sequence
  amplicon
gttacaatagactgtgtctattgcagaaggcaactacaacggacagagacaataggtggccgtatttacatagtagactaaccgtgtttaaatttc

SEQ ID NO: 1685 - DNA, 88 bp, Artificial Sequence
  amplicon
gttacaatagactgtgtctattgcagaaggcaactacaacggacagaggtgcatgagttgcctgaaaccattgtgtccagcagaaaaa

SEQ ID NO: 1686 - DNA, 91 bp, Artificial Sequence
  amplicon
gttacaatagactgtgtctattgcagaaggcaactacaacggacagagtaccactgacggaaaagtatccactactgaatctgttgccgac

SEQ ID NO: 1687 - DNA, 90 bp, Artificial Sequence
  amplicon
gttacaatagactgtgtctattgcagaaggcaactacaacggacagagtgtaacaaggcactgcaactagtagtagaagcgtcgcgggac

SEQ ID NO: 1688 - DNA, 98 bp, Artificial Sequence
  amplicon
aagacggagcctttgttgtggtgacactacacctatagtgcatttaaaagatggcattgtggcgagctagcgacaacatggtgtatttgcctcccccc

SEQ ID NO: 1689 - DNA, 79 bp, Artificial Sequence
  amplicon
acccagtaatctgcaatggccaattgtgaagacaataggtggccgtatttacatagtagactaaccgtgtttaaatttc

SEQ ID NO: 1690 - DNA, 74 bp, Artificial Sequence
  amplicon
acccagtaatctgcaatggccaattgtgaagtaccactgacggaaaagtatccactactgaatctgttgccgac

SEQ ID NO: 1691 - DNA, 79 bp, Artificial Sequence
  amplicon
acccagtaatctgcaatggccaattgtgaagatggcattgtggcgagctagcgacaacatggtgtatttgcctcccccc

SEQ ID NO: 1692 - DNA, 104 bp, Artificial Sequence
  amplicon
ggacagtggatatggcaatactgaagtggaaacttacgagacagagatgatacttggaaatatttacatagtagaattaaggtgtttacttttttaaatccatt

SEQ ID NO: 1693 - DNA, 89 bp, Artificial Sequence
  amplicon
ggacagtggatatggcaatactgaagtggaaacttacgagacagagcgcctgtgaagtatccattcctgaaattgttaacccactgcac

SEQ ID NO: 1694 - DNA, 115 bp, Artificial Sequence
  amplicon
atacatgatataaacctggactgtgtgttttgccaacgtggactgtacagatctgagatgatacttggaaatatttacatagtagaattaaggtgtttacttttttaaatccatt

SEQ ID NO: 1695 - DNA, 100 bp, Artificial Sequence
  amplicon
atacatgatataaacctggactgtgtgttttgccaacgtggactgtacagatctgagcgcctgtgaagtatccattcctgaaattgttaacccactgcac

SEQ ID NO: 1696 - DNA, 117 bp, Artificial Sequence
  amplicon
atacatgatataaacctggactgtgtgttttgccaacgtggactgtacagatctgaggtgcggaaaatgccaaaaaccattatgtccactggaaaagcaaaagcatgtagatgaaaa

SEQ ID NO: 1697 - DNA, 116 bp, Artificial Sequence
  amplicon
atacatgatataaacctggactgtgtgttttgccaacgtggactgtacagatctgagaccatctgcaactgtggtgtaagatgcatggaaaaaaaacaaccttgcaggacattact

SEQ ID NO: 1698 - DNA, 112 bp, Artificial Sequence
  amplicon
atacatgatataaacctggactgtgtgttttgccaacgtggactgtacagatctgagtgtcagtgcacagtatgccttgccattgaaagcaacaaagctgatttaagagtga

SEQ ID NO: 1699 - DNA, 89 bp, Artificial Sequence
  amplicon
cctgtacccagtgtactacacataatgttgcgccaatagtgcatttaaaagatgtggcgacctactgatgcaaaggtatacctgccccc

SEQ ID NO: 1700 - DNA, 96 bp, Artificial Sequence
  amplicon
cctgtacccagtgtactacacataatgttgcgccaatagtgcatttaaaaggattctcaaaaacgtaaaaccatagttcctaaagtttcaggtttg

SEQ ID NO: 1701 - DNA, 103 bp, Artificial Sequence
  amplicon
gccccaactgttccagaaacctataaaagaagatggctgattcagatgatacttggaaatatttacatagtagaattaaggtgtttacttttttaaatccatt

SEQ ID NO: 1702 - DNA, 88 bp, Artificial Sequence
  amplicon
gccccaactgttccagaaacctataaaagaagatggctgattcagcgcctgtgaagtatccattcctgaaattgttaacccactgcac

SEQ ID NO: 1703 - DNA, 83 bp, Artificial Sequence
  amplicon
gccccaactgttccagaaacctataaaagaagatggctgattcagatgtggcgacctactgatgcaaaggtatacctgccccc

SEQ ID NO: 1704 - DNA, 90 bp, Artificial Sequence
  amplicon
gccccaactgttccagaaacctataaaagaagatggctgattcaggattctcaaaaacgtaaaaccatagttcctaaagtttcaggtttg

SEQ ID NO: 1705 - DNA, 106 bp, Artificial Sequence
  amplicon
cacaagtggagactgtggaaggacccttacagatccaaatttaatgtatttacatagtagagtgacagtatttcaatttttaaatgcatttccatttgacccccat

SEQ ID NO: 1706 - DNA, 67 bp, Artificial Sequence
  amplicon
cacaagtggagactgtggaaggacccttacagtacctacagcaccccgtcaccctctactacaactg

SEQ ID NO: 1707 - DNA, 120 bp, Artificial Sequence
  amplicon
attcaggtattgtgtgtatattgtaaaaaggagttgtgtagagcagatccaaatttaatgtatttacatagtagagtgacagtatttcaatttttaaatgcatttccatttgacccccat

SEQ ID NO: 1708 - DNA, 81 bp, Artificial Sequence
  amplicon
attcaggtattgtgtgtatattgtaaaaaggagttgtgtagagcagtacctacagcaccccgtcaccctctactacaactg

SEQ ID NO: 1709 - DNA, 86 bp, Artificial Sequence
  amplicon
attcaggtattgtgtgtatattgtaaaaaggagttgtgtagagcaggtgtcatagatgtcagagaccacttgggcctgaagaaaag

SEQ ID NO: 1710 - DNA, 104 bp, Artificial Sequence
  amplicon
attcaggtattgtgtgtatattgtaaaaaggagttgtgtagagcagaaaaccaccaagacaacgtagtgaaacccaggtgtaataacgccatgcgtggtaatgt

SEQ ID NO: 1711 - DNA, 93 bp, Artificial Sequence
  amplicon
attcaggtattgtgtgtatattgtaaaaaggagttgtgtagagcaggtgttcgagtgttgtacagctcgcagtggaaagcagtggagacagcc

SEQ ID NO: 1712 - DNA, 120 bp, Artificial Sequence
  amplicon
ggaactgcaggcccaaacaccggagggcacctcagtgcaactaaaactgcgtttatagttcatttaaaagatggctttgtggcgtactaatgacagcaaagtgtatttaccacctgcacc

SEQ ID NO: 1713 - DNA, 95 bp, Artificial Sequence
  amplicon
catcggcaatggacagtgaagatccaaatttaatgtatttacatagtagagtgacagtatttcaatttttaaatgcatttccatttgacccccat

SEQ ID NO: 1714 - DNA, 56 bp, Artificial Sequence
  amplicon
catcggcaatggacagtgaagtacctacagcaccccgtcaccctctactacaactg

SEQ ID NO: 1715 - DNA, 71 bp, Artificial Sequence
  amplicon
catcggcaatggacagtgaagatggctttgtggcgtactaatgacagcaaagtgtatttaccacctgcacc

SEQ ID NO: 1716 - DNA, 128 bp, Artificial Sequence
  amplicon
ggaaactcagcagatgttacaggtagaagggcgccatgagactgaaacaccatgtagtcagtatagtggtggaagtgggggtggttgcagtcagtacagtagtggaagtgggggagagggtgttagtg

SEQ ID NO: 1717 - DNA, 91 bp, Artificial Sequence
  amplicon
gatataatattagaatgtgtgtactgcaagcaacagttactgcgacgtgaggtatatgactttgcttttcgggatttatgcatagtatata

SEQ ID NO: 1718 - DNA, 96 bp, Artificial Sequence
  amplicon
actaaaatgccctccattattaattacatctaacattaatgctggtacagattctaggtggccttatttacataatagattggtggtgtttacatt

SEQ ID NO: 1719 - DNA, 82 bp, Artificial Sequence
  amplicon
aagaactggaaatcctttttctcaaggacgtggtccagattaagtttgcacgaggacgaggacaaggaaaacgatggagact

SEQ ID NO: 1720 - DNA, 90 bp, Artificial Sequence
  amplicon
tattatgtcctacatctgtgtttagcagcaacgaagtatcctctcctgaaattattaggcagcacttggccaaccaccccgccgcgaccc

SEQ ID NO: 1721 - DNA, 101 bp, Artificial Sequence
  amplicon
tgtaatagtaacactacacccatagtacatttaaaaggtgatgctaatactttaaaatgtttaagatatagatttaaaaagcattgtacattgtatactgc

SEQ ID NO: 1722 - DNA, 55 bp, Artificial Sequence
  amplicon
aaccgttgtgtgatttgttaattaggtgtattaactgtcaaaagccactgtgtcc

SEQ ID NO: 1723 - DNA, 89 bp, Artificial Sequence
  amplicon
tatgtcttgttgcagatcatcaagaacacgtagagaaacccagctgtaatcatgcatggagatacacctacattgcatgaatatatgtt

SEQ ID NO: 1724 - DNA, 143 bp, Artificial Sequence
  amplicon
tatacaattattgctgatgcaggtgacttttatttacatcctagttattacatgttacgaaaacgacgtaaacgtttaccatattttttttcagatgtctctttggctgcctagtgaggccactgtctacttgcctcctgtcc

SEQ ID NO: 1725 - DNA, 109 bp, Artificial Sequence
  amplicon
gtccagctggacaagcagaaccggacagagcccattacaatattgtaaccttttgttgcaagtgtgactctacgcttcggttgtgcgtacaaagcacacacgtagacat

SEQ ID NO: 1726 - DNA, 52 bp, Artificial Sequence
  amplicon
tctcagaaaccataatctaccatggctgatcctgcaggtaccaatggggaag

SEQ ID NO: 1727 - DNA, 72 bp, Artificial Sequence
  amplicon
gaagtggaagcaacacagattcaggtaactacaaatggcgaacatggcggcaatgtatgtagtggcggcagt

SEQ ID NO: 1728 - DNA, 86 bp, Artificial Sequence
  amplicon
gtatattgcaagacagtattggaacttacagaggtatttgaatttgcatttaaagatttatttgtggtgtatagagacagtatacc

SEQ ID NO: 1729 - DNA, 91 bp, Artificial Sequence
  amplicon
atatacatccagcaaaggataatagatggccatatttagaaagtagaataacagtatttgaatttccaaatgcatttccatttgataaaaa

SEQ ID NO: 1730 - DNA, 84 bp, Artificial Sequence
  amplicon
tgcgaggaactatggaatacagaacctactcactgctttaaaaaaggtggccaaacagtacaagtatattttgatggcaacaaa

SEQ ID NO: 1731 - DNA, 69 bp, Artificial Sequence
  amplicon
ctcagcttgttaaacagctacagcacaccccctcaccgtattccagcaccgtgtccgtgggcaccgcaa

SEQ ID NO: 1732 - DNA, 82 bp, Artificial Sequence
  amplicon
caaaagacggaaactctgtagtggtaacactacgcctataatacatttaaaaggtgacagaaacagtttaaaatgtttacgg

SEQ ID NO: 1733 - DNA, 99 bp, Artificial Sequence
  amplicon
atatcatccacctggcattggacaggtgcaggcaatgaaaaaacaggaatactgactgtaacataccatagtgaaacacaaagaacaaaatttttaaat

SEQ ID NO: 1734 - DNA, 106 bp, Artificial Sequence
  amplicon
tggaaaaactaactaacactgggttatacaatttattaataaggtgcctgcggtgccagaaaccgttgaatccagcagaaaaacttagacaccttaatgaaaaacg

SEQ ID NO: 1735 - DNA, 57 bp, Artificial Sequence
  amplicon
tatttttttgcagatggctttgtggcggcctagtgacaataccgtatatcttccacc

SEQ ID NO: 1736 - DNA, 87 bp, Artificial Sequence
  amplicon
gattattaactgttggtaatccatattttagggttcctgcaggtggtggcaataagcaggatattcctaaggtttctgcataccaat

SEQ ID NO: 1737 - DNA, 123 bp, Artificial Sequence
  amplicon
agttaatcatcaacatttaccagcccgacgagccgaaccacaacgtcacacaatgttgtgtatgtgttgtaagtgtgaagccagaattgagctagtagtagaaagctcagcagacgaccttcg

SEQ ID NO: 1738 - DNA, 82 bp, Artificial Sequence
  amplicon
caatggctgatccagaaggtacagacggggagggcacgggttgtaacggctggttttatgtacaagctattgtagacaaaaa

SEQ ID NO: 1739 - DNA, 70 bp, Artificial Sequence
  amplicon
ggaaacgcagcagatggtacaggtagaggagcaacaaacaacattaagttgtaatggtagtgacgggaca

SEQ ID NO: 1740 - DNA, 104 bp, Artificial Sequence
  amplicon
gaactaagattgaattgtgtctactgcaaaggtcagttaacagaaacagaggtattagattttgcatttacagatttaacaatagtatatagggacgacacacc

SEQ ID NO: 1741 - DNA, 97 bp, Artificial Sequence
  amplicon
atagatgtaaagcataaagctttaatgcagttaaaatgtcctcctttattgattacatctaatataaatgcaggtaaggatgacagatggccatacc

SEQ ID NO: 1742 - DNA, 111 bp, Artificial Sequence
  amplicon
ttccatttgacaaaaacggaaatccagtatatgaattaagtgataaaaactggaaatcctttttctcaaggacgtggtgcagattaaatttgcacgaggaagaggacaaag

SEQ ID NO: 1743 - DNA, 73 bp, Artificial Sequence
  amplicon
ttgtttttcctgaatctgtatttagcagtgacgaaatatcctttgctgggattgttacaaagctaccaacagc

SEQ ID NO: 1744 - DNA, 103 bp, Artificial Sequence
  amplicon
aacaagggctgtcagttgtcctgcaactacacctataatacacttaaaaggtgatgcaaatatattaaaatgtttaagatataggctgtcaaaatataaacaa

SEQ ID NO: 1745 - DNA, 36 bp, Artificial Sequence
  amplicon
tatatgtgatttgttaattaggtgtataacgtgtca

SEQ ID NO: 1746 - DNA, 72 bp, Artificial Sequence
  amplicon
aggtggacaggacgttgcatagcatgttggagaagacctcgtactgaaacccaagtgtaaacatgcgtggag

SEQ ID NO: 1747 - DNA, 112 bp, Artificial Sequence
  amplicon
ggggtgatttttatttgcaccctagttattatatgttaaaacgtcgacgtaaacgtgtatcatatttttttacagatgtctctgtggcggcctagcgaggctactgtctact

SEQ ID NO: 1748 - DNA, 104 bp, Artificial Sequence
  amplicon
ccagctggacaagcagaaccggacacatccaattacaatatcgttaccttttgttgtcagtgtaagtctacacttcgtttgtgtgtacagagcacacaagtaga

SEQ ID NO: 1749 - DNA, 84 bp, Artificial Sequence
  amplicon
tctactagactgtaactacaatggctgatccagcaggtacagatggggaggggacgggatgcaatggttggttttatgtagaag

SEQ ID NO: 1750 - DNA, 79 bp, Artificial Sequence
  amplicon
gcaatactgaagtggaaactcagcagatggtacaacaggtagaaagtcaaaatggcgacacaaacttaaatgacttaga

SEQ ID NO: 1751 - DNA, 97 bp, Artificial Sequence
  amplicon
gaactacagtgcgtggaatgcaaaaaacctttgcaacgatctgaggtatatgattttgcatttgcagatttaacagttgtatatagagagggaaatc

SEQ ID NO: 1752 - DNA, 108 bp, Artificial Sequence
  amplicon
aaatgtccaccactgcttcttacctcaaatacaaatgcaggcacagactctagatggccatatttacatagtagattaacagtatttgaatttaaaaatccattccca

SEQ ID NO: 1753 - DNA, 97 bp, Artificial Sequence
  amplicon
atgtgggaagtacatgtgggtggtcaggtaattgtttgtcctacgtctatatctagcaaccaaatatccactactgaaactgctgacatacagacag

SEQ ID NO: 1754 - DNA, 108 bp, Artificial Sequence
  amplicon
acaagcagcggactgtgtgtagttctaacgttgcacctatagtgcatttaaaaggtgaatcaaatagtttaaaatgtttaagatacagattaaaaccttataaagagt

SEQ ID NO: 1755 - DNA, 70 bp, Artificial Sequence
  amplicon
ttaaaaaacctttaaatgaaatattaattaggtgtattatatgtcaaagacctttgtgtcctcaagaaaa

SEQ ID NO: 1756 - DNA, 76 bp, Artificial Sequence
  amplicon
gcagggcgctgtgcggcgtgttggaggtcccgacgtagagaaactgcactgtgacgtgtaaaaacgccatgagagg

SEQ ID NO: 1757 - DNA, 112 bp, Artificial Sequence
  amplicon
gttttacatcctagttattttattttacgtcgcaggcgtaaacgttttccatatttttttacagatgtccgtgtggcggcctagtgaggccacagtgtacctgcctcctgta

SEQ ID NO: 1758 - DNA, 73 bp, Artificial Sequence
  amplicon
caataaacatcatctacaatggccgatcctgaaggtacaaatggggctgggatggggtgtactggttggtttg

SEQ ID NO: 1759 - DNA, 77 bp, Artificial Sequence
  amplicon
atggcaattctgaagtggaaatacagcagatacaacaggtagaggggcatgatacagttgaacaatgtagtatgggc

SEQ ID NO: 1760 - DNA, 88 bp, Artificial Sequence
  amplicon
ttgtttgaattgtgtatactgcaaacaagaattacagcggagtgaggtatatgactttgcatgctatgatttgtgtatagtatataga

SEQ ID NO: 1761 - DNA, 60 bp, Artificial Sequence
  amplicon
tacttattacatcaaatataaatgcaggcaaagatgacaggtggccatacttacatagca

SEQ ID NO: 1762 - DNA, 65 bp, Artificial Sequence
  amplicon
gataaaaactggaaatcctttttctcaaggacgtggtgcagattaaatttgcacgaggaagagga

SEQ ID NO: 1763 - DNA, 90 bp, Artificial Sequence
  amplicon
ggtcaggtaattgtttgtcctgaatctgtatttagcagcacagaactatccactgctgaaattgctacacagctacacgcctacaacacc

SEQ ID NO: 1764 - DNA, 106 bp, Artificial Sequence
  amplicon
ccggtgtggtagttgtagtacaactacacctatagtacatttaaaaggtgatgcaaatacattaaagtgtttaagatatagattgggtaaatataaagcattgtat

SEQ ID NO: 1765 - DNA, 85 bp, Artificial Sequence
  amplicon
aacagttatgtcatttattaattaggtgtattacatgtcaaaaaccgctgtgtccagttgaaaagcaaagacatttagaagaaaa

SEQ ID NO: 1766 - DNA, 98 bp, Artificial Sequence
  amplicon
attatttattaaaacgacgtcgtaaacgtatcccatatttttttgcagatgtctctgtggcggtctaacgaagccactgtctacctgcctccagtgtc

SEQ ID NO: 1767 - DNA, 68 bp, Artificial Sequence
  amplicon
aggctattagctgtgggtcacccatactatgctattaaaaaacaagattctaataaaatagcagtacc

SEQ ID NO: 1768 - DNA, 69 bp, Artificial Sequence
  amplicon
ctacaatggctgatcctgcaggtacagatgaaggggaggggacgggatgtaatggatggttttttgtag

SEQ ID NO: 1769 - DNA, 83 bp, Artificial Sequence
  amplicon
gcggatatggcaatatggaagtggaaacagctgaagtggaggaggtaactgtagcaactaatacaaatggggatgctgaaggg

SEQ ID NO: 1770 - DNA, 90 bp, Artificial Sequence
  amplicon
gcctgtgtctattgcagacgaccactacagcaaaccgaggtatatgaatttgcatttagtgatttatatgtagtatatagggacggggaa

SEQ ID NO: 1771 - DNA, 118 bp, Artificial Sequence
  amplicon
aaatataaaagtttactacaaatgaaatgtccaccattattaataacctccaataccaatcctgtggaagacgataggtggccatatttacgtagtaggctaacagtgtttaaatttc

SEQ ID NO: 1772 - DNA, 120 bp, Artificial Sequence
  amplicon
acacaagacggtacctcagttgtggtaacactacgcctataatacatttaaaaggtgacaaaaatggtttaaaatgtttaagatatagactacaaaaatatgacacattgtttgaaaata

SEQ ID NO: 1773 - DNA, 122 bp, Artificial Sequence
  amplicon
ctacattagaaaatataactaatacaaagttatataatttattaataaggtgcatgtgttgtctgaaaccgctgtgtccagcagaaaaattaagacacctaaatagcaaacgaagatttcat

SEQ ID NO: 1774 - DNA, 116 bp, Artificial Sequence
  amplicon
attatttgttgccattattgtattttttcctaaaaaaacgtaaacgtattccctattttttttcagatggctatgtggcggtctagtgacagcatggtgtatttgcctccaccttc

SEQ ID NO: 1775 - DNA, 94 bp, Artificial Sequence
  amplicon
actagccagacgggatgaaccacagcgtcacacaatacagtgttcgtgttgtaagtgtaacaacacactgcagctggtagtagaagcctcacgg

SEQ ID NO: 1776 - DNA, 87 bp, Artificial Sequence
  amplicon
accagtaacctgctatggccaatcgtgaaggtacagacggggatgggtcgggatgtaacggatggtttctagtacaggcaatagtag

SEQ ID NO: 1777 - DNA, 68 bp, Artificial Sequence
  amplicon
gaagtggaagctgcagagactcaggtaactgtaaacactaatgcggaaaatggcggcagtgtacatag

SEQ ID NO: 1778 - DNA, 85 bp, Artificial Sequence
  amplicon
atattgcaaagcaacattggaacgcacagaggtatatcaatttgcttttaaagatttatgtatagtgtatagagactgtatagca

SEQ ID NO: 1779 - DNA, 62 bp, Artificial Sequence
  amplicon
tattaacatccaatattgatccagcaaaagataataaatggccatatttagaaagtagggtg

SEQ ID NO: 1780 - DNA, 43 bp, Artificial Sequence
  amplicon
ctcagattgttagacagctacaacacgcctccacgtcgacccc

SEQ ID NO: 1781 - DNA, 79 bp, Artificial Sequence
  amplicon
aagaaggaaagtgtgtagtggtaacactacgcctataatacacttaaaaggtgacaaaaacagtttgaaatgtttaaga

SEQ ID NO: 1782 - DNA, 110 bp, Artificial Sequence
  amplicon
atatcctccacctggcattggacaggttgtaataaaaacactggtatattaactgtaacatataatagtgaggtacaaagaaatacctttttggatgtagttactattcc

SEQ ID NO: 1783 - DNA, 75 bp, Artificial Sequence
  amplicon
aaaaataactaatacagagttgtataatttgttaataaggtgcctgcggtgccagaaaccattgaacccagcaga

SEQ ID NO: 1784 - DNA, 97 bp, Artificial Sequence
  amplicon
ttattattttcctaaaaaacgtaaacgtattccctatttttttgcagatggctttgtggcggcctagtgacagtacggtatatcttccaccaccttc

SEQ ID NO: 1785 - DNA, 99 bp, Artificial Sequence
  amplicon
gcccgacgagccgaaccacagcgtcacaaaattttgtgtgtatgttgtaagtgtgacggcagaattgagcttacagtagagagctcggcagaggacctt

SEQ ID NO: 1786 - DNA, 72 bp, Artificial Sequence
  amplicon
ccgtggtgtgcaactaaccaataatctacaatggcggatccagaaggtaccgacggggagggaacggggtgt

SEQ ID NO: 1787 - DNA, 86 bp, Artificial Sequence
  amplicon
cacaagtggaaactgtggaagcaacgttgcaggtagatgggcaacatggcggttcacagaacagtgtgtgtagtagcggggggggc

SEQ ID NO: 1788 - DNA, 113 bp, Artificial Sequence
  amplicon
gtttctatgcacaatatacaggtagtgtgtgtgtattgtaaaaaggaattatgtagagcagatgtatataatgtagcatttactgaaattaagattgtatatagggataataa

SEQ ID NO: 1789 - DNA, 88 bp, Artificial Sequence
  amplicon
ataaatccacaagaggatgcaaacctaatgtatttacatacaagggtaacagtattaaagtttttaaatacatttccatttgataaca

SEQ ID NO: 1790 - DNA, 106 bp, Artificial Sequence
  amplicon
tatggtactgtaataacatgtcctgaatatgtatctagtacctgcagcgacgcgttatccactactacaactgttgaacaactatcaaacaccccaacgaccaatc

SEQ ID NO: 1791 - DNA, 93 bp, Artificial Sequence
  amplicon
gtgcaactcagactgcgtttatagtgcatttaaaaggtgatacaaattgtttaaaatgttttagatacagatttacaaaacacaaagggttat

SEQ ID NO: 1792 - DNA, 118 bp, Artificial Sequence
  amplicon
gtatggtactacattagaggcaattactaaaaaaagcttatatgatttatcgataaggtgtcatagatgtcaaagaccacttgggcctgaagaaaagcaaaaattggtggacgaaaaa

SEQ ID NO: 1793 - DNA, 74 bp, Artificial Sequence
  amplicon
cgccgtaaacgtataccctatttttttacagatggcattgtggcgcactaatgacagcaaggtgtatttgccac

SEQ ID NO: 1794 - DNA, 95 bp, Artificial Sequence
  amplicon
gacgggctggacaggctacgtgttacagaattgaagctccgtgttgcaggtgttcaagtgtagtacaactggcagtggaaagcagtggagacacc

SEQ ID NO: 1795 - DNA, 109 bp, Artificial Sequence
  amplicon
gcccgtgttgtgcgaacaactagcaacggcgatggactgtgaaggtacagaggatgagggggcggggtgtaatgggtggttttttgttgaagcaatagtagaaaaaaaa

SEQ ID NO: 1796 - DNA, 84 bp, Artificial Sequence
  amplicon
cggctatggcaatagtgaagtggaagcgcagcagatggcagaccaggtagacgggcaaaatggcgactggcaaagtaacagtag

SEQ ID NO: 1797 - DNA, 78 bp, Artificial Sequence
  amplicon
gcagtgtgtgcagtgcaaaaaagagctacaacgaagagaggtatacaagtttctatttacagatttacgaatagtata

SEQ ID NO: 1798 - DNA, 60 bp, Artificial Sequence
  amplicon
taattttaacaacaaatacaaatgcaggaacagatcctaggtggccatatttacatagta

SEQ ID NO: 1799 - DNA, 82 bp, Artificial Sequence
  amplicon
ggtggtcaggtaattgtttgtcctgcatctgtatctagtaacgaagtatccactactgaaactgctgtccacctatgcaccg

SEQ ID NO: 1800 - DNA, 108 bp, Artificial Sequence
  amplicon
acaaaggacgggttgcacatacaacttgtactgcacctataatacacctaaaaggtgatcctaatagtttaaaatgtttaagatatagggtaaaaacacataaaagtt

SEQ ID NO: 1801 - DNA, 69 bp, Artificial Sequence
  amplicon
aaaaaaaccattaagtgaaataactattagatgtataatttgtcaaacgccattatgtcctgaagaaaa

SEQ ID NO: 1802 - DNA, 109 bp, Artificial Sequence
  amplicon
ataatattatgggtcgttggacagggcgctgttcagagtgttggagaccccgacctgtgacccaagtgtaacgtcatgcgtggagacaaagcaactataaaagattata

SEQ ID NO: 1803 - DNA, 67 bp, Artificial Sequence
  amplicon
acgttttccatatttttttacagatgtccgtgtggcggcctagtgaggccactgtgtacctgcctcc

SEQ ID NO: 1804 - DNA, 82 bp, Artificial Sequence
  amplicon
cgattactaacagtaggacatccctatttttctattaaaaacaccagtagtggtaatggtaaaaaagttttagttcccaagg

SEQ ID NO: 1805 - DNA, 109 bp, Artificial Sequence
  amplicon
gaccggccagatggacaagcagaacaagccacaagcaattactacattgtgacatattgtcacagttgtgatagcacactacggctatgcattcatagcactgcgacgg

SEQ ID NO: 1806 - DNA, 114 bp, Artificial Sequence
  amplicon
gtgtgccccggctgtgcacggctataaacaaccctgcaatggaggaccctgaaggtacagagggcgaaagggagggatgtacaggctggtttgaagtagaggcaataatagaaa

SEQ ID NO: 1807 - DNA, 70 bp, Artificial Sequence
  amplicon
cattggaaactctggaaacaccagaacaggtagatgaagaggtacagggacgtgggtgcgggaatacaca

SEQ ID NO: 1808 - DNA, 117 bp, Artificial Sequence
  amplicon
atacctttaattgatcttagattatcatgtgtatattgcaaaaaagaactaacacgtgctgaggtatataattttgcatgcactgaattaaaattagtgtatagggatgattttcct

SEQ ID NO: 1809 - DNA, 91 bp, Artificial Sequence
  amplicon
atttccattagataataatggtaatcctgtatatgaattaagtaatgtaaactggaaatgtttctttacaaggacgtggtccagattaaat

SEQ ID NO: 1810 - DNA, 99 bp, Artificial Sequence
  amplicon
actacgcctgtagtacatttaaaaggtgaacctaacagattaaaatgttgtagatatcgatttcaaaaatataaaacattgtttgtggatgtaacatca

SEQ ID NO: 1811 - DNA, 57 bp, Artificial Sequence
  amplicon
ataactaaaaaacagttatgtgatttattaataaggtgctacagatgtcaaagtccg

SEQ ID NO: 1812 - DNA, 98 bp, Artificial Sequence
  amplicon
atctaatagcacatggttggaccgggtcatgtttggggtgctggagacaaacatctagagaacctagagaatctacagtataatcatgcatggtaaag

SEQ ID NO: 1813 - DNA, 96 bp, Artificial Sequence
  amplicon
cctgtgtatttttttagacgtaggcgccgtaaacgtattccctatttttttgcagatggcgacgtggcggcctagtgaaaataaggtgtatctacc

SEQ ID NO: 1814 - DNA, 77 bp, Artificial Sequence
  amplicon
ttgcttgccgtaggacatccctattactctgtgactaaggacaataccaaaacaaacattcccaaagttagtgcata

SEQ ID NO: 1815 - DNA, 75 bp, Artificial Sequence
  amplicon
catacgtgttacctaatacacgtaccttgttgtgagtgtaagtttgtggtgcagttggacattcagagtaccaaa

SEQ ID NO: 1816 - DNA, 69 bp, Artificial Sequence
  amplicon
gcgcatcaagtaactaactgcaatggcgtcacctgaaggtacagatggggaggggaagggatgttgtgg

SEQ ID NO: 1817 - DNA, 68 bp, Artificial Sequence
  amplicon
atggcaatactgaagtggaaactgagcagatggcacaccaggtagaaagccaaaatggcgacgcagac

SEQ ID NO: 1818 - DNA, 111 bp, Artificial Sequence
  amplicon
gtgcatgaaatcgaattgaaatgcgttgaatgcaaaaagactttgcagcgatctgaggtatatgactttgtatttgcagatttaagaatagtgtatagagatggaaatcca

SEQ ID NO: 1819 - DNA, 111 bp, Artificial Sequence
  amplicon
aaaacatagggcattagtacaattaaaatgtccaccattaataattacctcaaatacaaatgcaggcaaagattcacgatggccatatttgcacagtagactaacagtatt

SEQ ID NO: 1820 - DNA, 83 bp, Artificial Sequence
  amplicon
gtcgggtaattgtatgtcctacatctatacctagtgatcaaatatccactactgaaactgctgacccaaagaccaccgaggcc

SEQ ID NO: 1821 - DNA, 119 bp, Artificial Sequence
  amplicon
gtacatacaaagggcggaacgtgtgtagttctaaagtttcacctatcgtgcatttaaaaggtgacccaaatagtttaaaatgtttaagatatagattaaaaccatttaaagacttatac

SEQ ID NO: 1822 - DNA, 91 bp, Artificial Sequence
  amplicon
ctaaaaaagtgtttaaatgaaatattaattagatgtattatttgtcaaagaccattgtgtccacaagaaaaaaaaaggcatgtggatttaa

SEQ ID NO: 1823 - DNA, 90 bp, Artificial Sequence
  amplicon
tattttgcgtcgcagacgtaaacgttttccatatttttttgcagatgtccgtgtggcggcctagtgaggccactgtgtacctgcctcctg

SEQ ID NO: 1824 - DNA, 97 bp, Artificial Sequence
  amplicon
gtgtgccctagctgtgcacagcaataaacaccatctgcaatggatgaccctgaaggtacaaacggggtaggggcgggctgtactggctggtttgagg

SEQ ID NO: 1825 - DNA, 81 bp, Artificial Sequence
  amplicon
gcggctatggctattctgaagtggaaatgctcgagactcaggtaaccgtggagaatactggaaatggggatagcaatggca

SEQ ID NO: 1826 - DNA, 72 bp, Artificial Sequence
  amplicon
ggaactgcaagaaagagaggtatttgaatttgcttttaatgacttatttatagtgtatagagactgtacacc

SEQ ID NO: 1827 - DNA, 95 bp, Artificial Sequence
  amplicon
attaaatgtccaccaatgcttattacatcaaatacaaatccagttacagataacaggtggccatatttaaatagcagattaatggtatttaaatt

SEQ ID NO: 1828 - DNA, 114 bp, Artificial Sequence
  amplicon
caacccgcgacggcacatcccttgcagtaacactacgcctataatacacttaaaaggtgacaaaaatggccttaagtgtttaaggtatagattaagaaaagtacactggttatt

SEQ ID NO: 1829 - DNA, 74 bp, Artificial Sequence
  amplicon
ctatttttttacagatggctctatggcgttctagtgacaacaaggtgtatctacctccaccttcggtagctaag

SEQ ID NO: 1830 - DNA, 120 bp, Artificial Sequence
  amplicon
cacgcaggcagttccagacttcttacagttggacatccatattttaaagtacctaaaggtggtaatggtagacaggatgttcctaaggtgtctgcatatcaatacagagtatttagggtt

SEQ ID NO: 1831 - DNA, 92 bp, Artificial Sequence
  amplicon
agctagacgagctgaaccacagcgtcacaacattgtgtgtgtgtgttgtaagtgtaataatcaacttcagctagtagtagaaacctcgcaag

SEQ ID NO: 1832 - DNA, 99 bp, Artificial Sequence
  amplicon
gcagcaaaccagtaacctgcaatggccgattcggaaggtacagatggggaagggacggggtgcaatggatggttttttgtgcaggcaatagtagataaa

SEQ ID NO: 1833 - DNA, 85 bp, Artificial Sequence
  amplicon
cattggaaacattggaaacatcacaacaggtagaatacgaaaagggaaatgggtgcgggagctcacaaaatggaggctcgcaaaa

SEQ ID NO: 1834 - DNA, 115 bp, Artificial Sequence
  amplicon
aatacctttacttgatcttagattatcatgtgtatactgcaaaaaggaacttacaagtttagagctatataggtttgcatgtattgagttaaaactagtatatagaaacaattgg

SEQ ID NO: 1835 - DNA, 111 bp, Artificial Sequence
  amplicon
ggggtggattacagaggcatatattatatgcatgatggccacaaaacatattacacagactttgaacaggaggccaaaaaatatgggtgtacaaacatatgggaagtacat

SEQ ID NO: 1836 - DNA, 102 bp, Artificial Sequence
  amplicon
ggtgataaaactacgcctgtaatccatttaaaaggtgaagctaatagattaaagtgttgtagatacagatttcaaaaatataaaacattatttacagatgta

SEQ ID NO: 1837 - DNA, 64 bp, Artificial Sequence
  amplicon
gttatctgatttatcaataaggtgctaccgatgtcaatgtccgttaacaccggaggaaaaacaa

SEQ ID NO: 1838 - DNA, 90 bp, Artificial Sequence
  amplicon
tatttttttaaacgtaggcgccgtaaacgtattccctatttttttgcagatggcgatgtggcggcctagtgacaataaggtgtacctacc

SEQ ID NO: 1839 - DNA, 77 bp, Artificial Sequence
  amplicon
caacataagtgttacctaattcacgtaccttgttgtaagtgtgagttggtggtgcagttggacattcagagtaccaa

SEQ ID NO: 1840 - DNA, 69 bp, Artificial Sequence
  amplicon
gcgcatcatctaaataactgcaatggcatcacctgaaggtacagatggggaggggatgggatgttgtgg

SEQ ID NO: 1841 - DNA, 108 bp, Artificial Sequence
  amplicon
ccggacagcggctatggcaatatggaagtggaaactaactcggaggtaactgtagcacctaatataaatggggaggatggggaaaatgaaggggaaaatggcgacagt

SEQ ID NO: 1842 - DNA, 97 bp, Artificial Sequence
  amplicon
gttacaatagactgtgtctattgcagaaggcaactacaacggacagaggtatatgaatttgcctttagtgacctatgtgtagtgtatagagacgggg

SEQ ID NO: 1843 - DNA, 77 bp, Artificial Sequence
  amplicon
aataacatccaatactaaccctgtagaagacaataggtggccgtatttacatagtagactaaccgtgtttaaatttc

SEQ ID NO: 1844 - DNA, 78 bp, Artificial Sequence
  amplicon
accataactaatacaaagttatataatttattgataaggtgcatgagttgcctgaaaccattgtgtccagcagaaaaa

SEQ ID NO: 1845 - DNA, 80 bp, Artificial Sequence
  amplicon
atggcaacataatccattgtcctgactctatgtgcagtaccactgacggaaaagtatccactactgaatctgttgccgac

SEQ ID NO: 1846 - DNA, 77 bp, Artificial Sequence
  amplicon
aagacggagcctttgttgtggtgacactacacctatagtgcatttaaaaggtgacaaaaatggattaaaatgtctta

SEQ ID NO: 1847 - DNA, 121 bp, Artificial Sequence
  amplicon
tccaattattatttattaccattgttattctttttattaaaaaaacgtaaacaccttccttatttttttacagatggcattgtggcgagctagcgacaacatggtgtatttgcctcccccc

SEQ ID NO: 1848 - DNA, 87 bp, Artificial Sequence
  amplicon
cgggacgaacaacagcgtcacagaattcagtgtctgtgttgtaagtgtaacaaggcactgcaactagtagtagaagcgtcgcgggac

SEQ ID NO: 1849 - DNA, 83 bp, Artificial Sequence
  amplicon
acccagtaatctgcaatggccaattgtgaaggtaccgatggggacgggacggggtgtaacggatggttttttgtagaagcaat

SEQ ID NO: 1850 - DNA, 86 bp, Artificial Sequence
  amplicon
ggacagtggatatggcaatactgaagtggaaacttacgagacagaggtaccgggacttggggcaggggtagggtgtttacaaaatg

SEQ ID NO: 1851 - DNA, 115 bp, Artificial Sequence
  amplicon
atacatgatataaacctggactgtgtgttttgccaacgtggactgtacagatctgaggtatatgattttgcatttagtgatttgtgtattgtatatagaaaggataaaccatatg

SEQ ID NO: 1852 - DNA, 81 bp, Artificial Sequence
  amplicon
atcaaatacaaatcctaaagcagatgatacttggaaatatttacatagtagaattaaggtgtttacttttttaaatccatt

SEQ ID NO: 1853 - DNA, 81 bp, Artificial Sequence
  amplicon
atgggtggtcaggtaatatgttgtgctcctgtatctagcgcctgtgaagtatccattcctgaaattgttaacccactgcac

SEQ ID NO: 1854 - DNA, 118 bp, Artificial Sequence
  amplicon
cctgtacccagtgtactacacataatgttgcgccaatagtgcatttaaaaggtgacaaaaacagcttaaaatgttttagatatagattgcataaaggctattcacatttatttaaaaa

SEQ ID NO: 1855 - DNA, 99 bp, Artificial Sequence
  amplicon
aaatttaactaacaaacagttatgtaatattttaataaggtgcggaaaatgccaaaaaccattatgtccactggaaaagcaaaagcatgtagatgaaaa

SEQ ID NO: 1856 - DNA, 87 bp, Artificial Sequence
  amplicon
tattatttgttaaagcgcaaacgtaaacgtctgtcatattcttttacagatgtggcgacctactgatgcaaaggtatacctgccccc

SEQ ID NO: 1857 - DNA, 77 bp, Artificial Sequence
  amplicon
tggctgtgggacacccatattttcctatcaaggattctcaaaaacgtaaaaccatagttcctaaagtttcaggtttg

SEQ ID NO: 1858 - DNA, 116 bp, Artificial Sequence
  amplicon
cagccatctagacagacaagctgaacgagagtgttacagaatagttactgactgcacgaagtgtcagtgcacagtatgccttgccattgaaagcaacaaagctgatttaagagtga

SEQ ID NO: 1859 - DNA, 96 bp, Artificial Sequence
  amplicon
gccccaactgttccagaaacctataaaagaagatggctgattcaggtaattgggaagggaggtgtacgggatggtttaatgtagaagccattgtag

SEQ ID NO: 1860 - DNA, 86 bp, Artificial Sequence
  amplicon
cacaagtggagactgtggaaggacccttacaggtagatgggcaaaatgacgggtcacaacatagtatgtgtagtggcggggggagc

SEQ ID NO: 1861 - DNA, 90 bp, Artificial Sequence
  amplicon
attcaggtattgtgtgtatattgtaaaaaggagttgtgtagagcagatgtgtataatgtagcatttacagaacttaggattgtatatagg

SEQ ID NO: 1862 - DNA, 122 bp, Artificial Sequence
  amplicon
ttgtatgcccaccattgcttattacctcaaatatcaatccaaaagaagatccaaatttaatgtatttacatagtagagtgacagtatttcaatttttaaatgcatttccatttgacccccat

SEQ ID NO: 1863 - DNA, 79 bp, Artificial Sequence
  amplicon
tatatgtgtggcaatgtaataacatgtcctgaatatgtatctagtacctacagcaccccgtcaccctctactacaactg

SEQ ID NO: 1864 - DNA, 127 bp, Artificial Sequence
  amplicon
ggaactgcaggcccaaacaccggagggcacctcagtgcaactaaaactgcgtttatagttcatttaaaaggtgcaacaaattgtttaaaatgtttaagatacagatttgcaaaacatagaaatttgt

SEQ ID NO: 1865 - DNA, 82 bp, Artificial Sequence
  amplicon
agaggccattactaacaaaagtttatatgaattattaataaggtgtcatagatgtcagagaccacttgggcctgaagaaaag

SEQ ID NO: 1866 - DNA, 98 bp, Artificial Sequence
  amplicon
catatttgttacgcaaacgccgtaaacgtataccctatttttttgcagatggctttgtggcgtactaatgacagcaaagtgtatttaccacctgcacc

SEQ ID NO: 1867 - DNA, 106 bp, Artificial Sequence
  amplicon
cagccagccagacaagctggacaggatacgtgttacagaattaaagtgcactgttgcaggtgttcgagtgttgtacagctcgcagtggaaagcagtggagacagcc

SEQ ID NO: 1868 - DNA, 56 bp, Artificial Sequence
  amplicon
catcggcaatggacagtgaaggtacagaggatgagggggcggggtgtaccgggtgg

SEQ ID NO: 1869 - DNA, 77 bp, Artificial Sequence
  amplicon
ctgcatttggacttacacccagtatagctgacagtataaaaacactattacaacaatattgtttatatttacacatt

SEQ ID NO: 1870 - DNA, 77 bp, Artificial Sequence
  amplicon
gtgcttttgtgtgtctgcctattaatacgtccgctgcttttgtctgtgtctacatacacatcattaataatattggt

SEQ ID NO: 1871 - DNA, 83 bp, Artificial Sequence
  amplicon
cgtggtcagccattaggtgtgggcattagtggccatcctttattaaataaattggatgacacagaaaatgctagtgcttatgc

SEQ ID NO: 1872 - DNA, 106 bp, Artificial Sequence
  amplicon
caaccgaattcggttgcatgctttttggcacaaaatgtgtttttttaaatagttctatgtcagcaactatggtttaaacttgtacgtttcctgcttgccatgcgtg

SEQ ID NO: 1873 - DNA, 76 bp, Artificial Sequence
  amplicon
gatttaaaacactaatacagccatttatattatatgcccatattcaatgtctagactgtaaatggggagtattaat

SEQ ID NO: 1874 - DNA, 60 bp, Artificial Sequence
  amplicon
tatattgtggtaataacgtcccctgccacagcattcacagtatatgtattttgtttttta

SEQ ID NO: 1875 - DNA, 83 bp, Artificial Sequence
  amplicon
ctgtagattataagcagacacagttatgtattttgggctgtgcccctgctattggggaacactgggctaaaggcactgcttgt

SEQ ID NO: 1876 - DNA, 75 bp, Artificial Sequence
  amplicon
gtgtttgtggtatgggtgttgcttgttgggctatatattgtcctgtatttcaagttataaaactgcacaccttac

SEQ ID NO: 1877 - DNA, 121 bp, Artificial Sequence
  amplicon
aaacagtattacagcatagttttaatgacacaacatttgatttgtcccaaatggtacaatgggcatatgacaatgatgttatggatgatagtgaaattgcctataaatatgcacaattagc

SEQ ID NO: 1878 - DNA, 99 bp, Artificial Sequence
  amplicon
cttttgtgtgctactatttgtgtgtcttgtcatacgtccacttgtgctgtctgtgtcggtatatgcaacactactattattaattgtgattttatgggt

SEQ ID NO: 1879 - DNA, 78 bp, Artificial Sequence
  amplicon
ggtttagaggtaggtcgcgggcagccattaggtgtaggtattagtggtcatccattattaaataaatttgatgacact

SEQ ID NO: 1880 - DNA, 112 bp, Artificial Sequence
  amplicon
atatacaccctattagtaacatactattactattttataaactattgttcctacttgttcctacttgttcctgctcctcccaatagtcatgtacttatttctgcctataatt

SEQ ID NO: 1881 - DNA, 119 bp, Artificial Sequence
  amplicon
tattggtttagaacagcaatgtcaaacattagtgatgtacaaggtacaacacctgaatggatagatagactaactgttttacaacatagctttaatgataatatatttgatttaagtga

SEQ ID NO: 1882 - DNA, 97 bp, Artificial Sequence
  amplicon
ggtgttggtattgctgctttgggtgtttgtgggatctcctttaaaaatttttttttgctatttgttgtttttatatttaccaatgatgtgtattaat

SEQ ID NO: 1883 - DNA, 82 bp, Artificial Sequence
  amplicon
ggcagccattaggcgttggcataagtggtcatcctttattaaacaaatttgatgacactgaaaccggtaacaagtatcctgg

SEQ ID NO: 1884 - DNA, 90 bp, Artificial Sequence
  amplicon
caatgtacctacctttatttccctatatttgtagtacctacatgtttagtattgctttaccttttgacatactagtgtccatattgtaca

SEQ ID NO: 1885 - DNA, 74 bp, Artificial Sequence
  amplicon
aatacaaccaccaaaattacgtagtaccccagctgcgttatattggtttaaaacagcaatgtcaaatattagtg

SEQ ID NO: 1886 - DNA, 104 bp, Artificial Sequence
  amplicon
tactcagcattaatattactggttttaatactgtgggttactgtagcaacaccactacgttgcttttgttgttttctttgctttttgtatatacctatgggaat

SEQ ID NO: 1887 - DNA, 86 bp, Artificial Sequence
  amplicon
atggattataaacaaacacaattgtgtttaataggttgtaggcctcctataggtgaacattggggaaaaggcacaccttgtaatgc

SEQ ID NO: 1888 - DNA, 92 bp, Artificial Sequence
  amplicon
ttaatccttgtgttcctgatatatattgtttgccaactttatattggcttttgccaatctttaaacttgattcatcttgcagtattagtcat

SEQ ID NO: 1889 - DNA, 95 bp, Artificial Sequence
  amplicon
acattgttacatgttccagaaagttgtatgcttctggagcctcctaaactgcgcagccctgtagcagcactatattggtatcgcacaggtatatc

SEQ ID NO: 1890 - DNA, 75 bp, Artificial Sequence
  amplicon
atatatgttgcaatgtcccgcttttgccgtctgtgcatgtgtgtgcgtatgtgtggataattgtgtttgtgttta

SEQ ID NO: 1891 - DNA, 102 bp, Artificial Sequence
  amplicon
ttagtggacacccattatataatagacaggatgatactgaaaactcaccattttcatcaaccaccaataaggacagtagggataatgtgtctgtggattata

SEQ ID NO: 1892 - DNA, 117 bp, Artificial Sequence
  amplicon
cggtcgtgggttgagcattttttttaaactagtggaaaccacctttctcagcaaaaacatgtctttaccttaggttcaccctgcatagttggcactggtaacagttttactggcgcg

SEQ ID NO: 1893 - DNA, 83 bp, Artificial Sequence
  amplicon
gtttagattgtaaatggggagtattaatattagctttattaagatataaatgtggcaaaaatagactaactgttgcaaaaggc

SEQ ID NO: 1894 - DNA, 127 bp, Artificial Sequence
  amplicon
gtttctttttatagttgttattacatccccattaacagcatttgctgtatacatttgttgctatttactacctatgtttgtattacatatgcatgctttacacaccatacaataattactataatgt

SEQ ID NO: 1895 - DNA, 79 bp, Artificial Sequence
  amplicon
cgtgggcagcctttaggtattggcctaagtggccatccattttataataaattggatgatacagaaagtgctcatgcag

SEQ ID NO: 1896 - DNA, 125 bp, Artificial Sequence
  amplicon
tgtgaccttttaaacataatacctaaactggcacatttacaacccctacatagtttaacctactggcgcgccttcttggcgtacatgtggcacacctggtattagtcattttcctgtccaggtgt

SEQ ID NO: 1897 - DNA, 90 bp, Artificial Sequence
  amplicon
gctaaatttttaagatatcaaggtgtaaactttatgtcctttattcaaatgtttaaacagtttttaaaaggaacaccaaaacacaattgc

SEQ ID NO: 1898 - DNA, 88 bp, Artificial Sequence
  amplicon
tagacatattgtaaccattgcagtgtttattattttgctatttgtgctttgcttgtgtgtgtgtcttgtgttgtgttgtttgttgccg

SEQ ID NO: 1899 - DNA, 94 bp, Artificial Sequence
  amplicon
gccagatcctttggccaaatataaattttgggatgttgatttaaaggaacgattttctttagatttagaccaatttgcattgggtcgcaagttt

SEQ ID NO: 1900 - DNA, 89 bp, Artificial Sequence
  amplicon
gggtggtgtttcggtggcgtccctattgccctacccattttttgcagcacaacagtttatatttgtgctatttagttatactttgtagc

SEQ ID NO: 1901 - DNA, 106 bp, Artificial Sequence
  amplicon
tattaatacagccctatagcatatatgcccatttgcaatgtttaacatgtgacagaggcgtgcttatactgctgctaattaggtttaaatgtggaaaaaacagatt

SEQ ID NO: 1902 - DNA, 87 bp, Artificial Sequence
  amplicon
ttgtgtatatataacaatgttaggattatttgtattttgttttattttgcttatggtgttttgtgcagtgcttaggccgctcttgct

SEQ ID NO: 1903 - DNA, 80 bp, Artificial Sequence
  amplicon
aaaagaaaagttttctgcagatttagatcagtttcctttaggtaggaagtttttgttacaggcagggctacaggctaggc

SEQ ID NO: 1904 - DNA, 89 bp, Artificial Sequence
  amplicon
ctttggttgtccttggcacagtaacaactatttttatataagtttcagcaaactgcttaatcctttggtttcctgcagtccactggtct

SEQ ID NO: 1905 - DNA, 92 bp, Artificial Sequence
  amplicon
atatgcacaattagcagatgtagacagcaatgcacaagcctttttaaaaagcaatatgcaggcaaaatatgtaaaggattgtggaataatgt

SEQ ID NO: 1906 - DNA, 114 bp, Artificial Sequence
  amplicon
tttttgtgttattggtgtgtttgcgctttgcttttgtgtttgtttgcttgtgtgtcatgttgtcccgcttttgctatctgcctctgtgttttccagttgtatattattaataat

SEQ ID NO: 1907 - DNA, 82 bp, Artificial Sequence
  amplicon
cattggactaaaggtgctgtgtgtaagtccacacaagttaccacaggggactgcccgcctcttgcattaattaatacaccta

SEQ ID NO: 1908 - DNA, 119 bp, Artificial Sequence
  amplicon
gccattatttaaactaaaaggaattcggttgcatggcctagtgccattatttaaaccaaaaggcccttttcagcagaacagttaatcctttggcatattgccgtttcctgtgttttata

SEQ ID NO: 1909 - DNA, 122 bp, Artificial Sequence
  amplicon
gtgaaaaaacaaatgatggaggtaattggagaccaatagtacaatttttaagatatcaaaatattgaatttacagcatttttagttgcatttaaacagtttttacaaggtgtaccaaaaaaa

SEQ ID NO: 1910 - DNA, 118 bp, Artificial Sequence
  amplicon
gtgttgctgctttgggtgtctgtggggtcggctctacgaatttttttctgttacttaatatttttatatataccaatgatgtgtattaattttcatgcacaatacttaacccaacaag

SEQ ID NO: 1911 - DNA, 83 bp, Artificial Sequence
  amplicon
ggccttgaaataggtaggggacagccattgggtgttggcgtaagtggtcatccttatttaaataaatttgatgacactgaaac

SEQ ID NO: 1912 - DNA, 77 bp, Artificial Sequence
  amplicon
tgccctaccctgccctgcctattatgcatacctatgtaatagtatttgtatgatatgtattttatagtttttaacag

SEQ ID NO: 1913 - DNA, 109 bp, Artificial Sequence
  amplicon
ttgcgtagtggtgttgcagcactatattggtacagaacaggaatgtccaatattagtgaagttataggggaaacgcccgaatggatacaaagactaacaattatacaac

SEQ ID NO: 1914 - DNA, 106 bp, Artificial Sequence
  amplicon
caatctgtctatatgtgtgcatatacatggttactagtatttgtgtatattgtggttatcacctcctcatatgagtgttttttactatatatattgttttttataa

SEQ ID NO: 1915 - DNA, 86 bp, Artificial Sequence
  amplicon
gaacactggacaaagggcactgcttgtaagcctactactgtggttcagggcgattgtcctccactagaattaataaatacaccaat

SEQ ID NO: 1916 - DNA, 79 bp, Artificial Sequence
  amplicon
ctgtccctttattgtttctttgtccttattacacattattacacattgccctacttacataggtgtgtttgttccttca

SEQ ID NO: 1917 - DNA, 114 bp, Artificial Sequence
  amplicon
tttttaaaaagtaatatgcaagcaaaatatgtaaaggattgtggaataatgtgtagacattacaaaagggcacagcaacagcaaatgaatatgtgccagtggataaagcatata

SEQ ID NO: 1918 - DNA, 124 bp, Artificial Sequence
  amplicon
ttgtcccgcttttgctatctgcatctttatttacaagttgtcttatactaattattttattttggtttgttgtggctacatcattttttgatacttttatactgtttttactatttttttatat

SEQ ID NO: 1919 - DNA, 123 bp, Artificial Sequence
  amplicon
ggtgctgggttaagtggtcatccattatttaataggctggatgacactgaggtctctaatttagcaggtaataatgttatagaagatagccgggacaatatatctgttgattgtaaacaaacc

SEQ ID NO: 1920 - DNA, 88 bp, Artificial Sequence
  amplicon
atgttatattaaataggttgtttgtatgcactatagtaacacaccaaactccattttagtgctgtacgccattttatgcatgcaaccg

SEQ ID NO: 1921 - DNA, 126 bp, Artificial Sequence
  amplicon
aaagattaaccataatacaacatggaatagatgatagtgtatttgatttatcagacatggtacaatgggcatttgataatgagttaacagatgacagtgatatagcatttcaatatgctatgttag

SEQ ID NO: 1922 - DNA, 91 bp, Artificial Sequence
  amplicon
atatatgttgcactgtcccgcttttgcagtctgtgcatgtgtgtgtgtatgtgtggatatttgtgtttgtgtttatattagttagaactac

SEQ ID NO: 1923 - DNA, 93 bp, Artificial Sequence
  amplicon
gggggcagccattgggcgttggccttagtgggcatccactatataataggctggatgatactgaaaattccccgttttcctctaataaaactc

SEQ ID NO: 1924 - DNA, 82 bp, Artificial Sequence
  amplicon
tttacatataataggactgcaacatttcatacataatttgtagccctaccctaaggtgtgttacattatatgcaatatattt

SEQ ID NO: 1925 - DNA, 84 bp, Artificial Sequence
  amplicon
gactacgaagtacaccatgtgcattatattggtatagaactagtttatcaaatattagtgaaatagtaggagacacacctgagt

SEQ ID NO: 1926 - DNA, 94 bp, Artificial Sequence
  amplicon
acccatggttattggtattgattataataacctttatacatgtatcacaatcattgttaaaagtattttttttatatgttttggtattttatat

SEQ ID NO: 1927 - DNA, 97 bp, Artificial Sequence
  amplicon
ggggaacattggggtccaggcacgccatgtacttcacaaactgttaatactggtgattgtcccccactggaattaaagaacacccctatacaggatg

SEQ ID NO: 1928 - DNA, 97 bp, Artificial Sequence
  amplicon
aaaaagggcaaccgatttcggttgcacagtaaaacatgttttaatgtgttttgctgttgtagcaaaatagttgtactgtttttggcttcctgcaggc

SEQ ID NO: 1929 - DNA, 109 bp, Artificial Sequence
  amplicon
tacttttatagaacaggaatatcaaacattagtagcacatatggcgaaacaccagaatggattacaagacaaacacaactacagcacagttttgatgatagcacgtttg

SEQ ID NO: 1930 - DNA, 82 bp, Artificial Sequence
  amplicon
accattgcggtgtttttggtgtgtttatttgtgctttgcgtgtgtgtgtgtcttgtgttgtgttgtttgttgccgctattgc

SEQ ID NO: 1931 - DNA, 80 bp, Artificial Sequence
  amplicon
ggggtcagccgttaggtgttggccttagtggtcatcctttatttaataagtatgatgatactgaaaactctaggtttgcc

SEQ ID NO: 1932 - DNA, 112 bp, Artificial Sequence
  amplicon
taggtggcgtccctattgccctacccatatttgtggcttgcagcacacttgtatatatatgttcttgctgtattgcatgtaccacaggattccattttgttttttcctgcag

SEQ ID NO: 1933 - DNA, 79 bp, Artificial Sequence
  amplicon
tattggaaacacatgcgcctagctgctcgcggccgccaccgccgggccccggccgtccctggctcccctcctgcctcga

SEQ ID NO: 1934 - DNA, 86 bp, Artificial Sequence
  amplicon
tattggaaacacatgcgcctagcagcctcccgcgacgatgcccctcaacgttagcttcaccaacaggaactatgacctcgactacg

SEQ ID NO: 1935 - DNA, 86 bp, Artificial Sequence
  amplicon
tattggaaacacatgcgcctagaggaggaacaagaagatgaggaagaaatcgatgttgtttctgtggaaaagaggcaggctcctgg

SEQ ID NO: 1936 - DNA, 68 bp, Artificial Sequence
  amplicon
tattggaaacacatgcgcctagctgaccatactccctggagccttctcccgaggtgcgcgggtgacct

SEQ ID NO: 1937 - DNA, 64 bp, Artificial Sequence
  amplicon
tattggaaacacatgcgcctagtctgagcctgatggatttacagtgatcttcagtggtctgggg

SEQ ID NO: 1938 - DNA, 107 bp, Artificial Sequence
  amplicon
tattggaaacacatgcgcctagctccgggcagagcgcgtgtggcggccgagcacatgggcccgcgggccgggcgggctcggggcggccgggacgaggaggggcgacg

SEQ ID NO: 1939 - DNA, 82 bp, Artificial Sequence
  amplicon
tgtaatagtaacactacacccatagctgctcgcggccgccaccgccgggccccggccgtccctggctcccctcctgcctcga

SEQ ID NO: 1940 - DNA, 89 bp, Artificial Sequence
  amplicon
tgtaatagtaacactacacccatagcagcctcccgcgacgatgcccctcaacgttagcttcaccaacaggaactatgacctcgactacg

SEQ ID NO: 1941 - DNA, 89 bp, Artificial Sequence
  amplicon
tgtaatagtaacactacacccatagaggaggaacaagaagatgaggaagaaatcgatgttgtttctgtggaaaagaggcaggctcctgg

SEQ ID NO: 1942 - DNA, 71 bp, Artificial Sequence
  amplicon
tgtaatagtaacactacacccatagctgaccatactccctggagccttctcccgaggtgcgcgggtgacct

SEQ ID NO: 1943 - DNA, 67 bp, Artificial Sequence
  amplicon
tgtaatagtaacactacacccatagtctgagcctgatggatttacagtgatcttcagtggtctgggg

SEQ ID NO: 1944 - DNA, 110 bp, Artificial Sequence
  amplicon
tgtaatagtaacactacacccatagctccgggcagagcgcgtgtggcggccgagcacatgggcccgcgggccgggcgggctcggggcggccgggacgaggaggggcgacg

SEQ ID NO: 1945 - DNA, 94 bp, Artificial Sequence
  amplicon
tctcagaaaccataatctaccatggctgatcctgcagctgctcgcggccgccaccgccgggccccggccgtccctggctcccctcctgcctcga

SEQ ID NO: 1946 - DNA, 101 bp, Artificial Sequence
  amplicon
tctcagaaaccataatctaccatggctgatcctgcagcagcctcccgcgacgatgcccctcaacgttagcttcaccaacaggaactatgacctcgactacg

SEQ ID NO: 1947 - DNA, 101 bp, Artificial Sequence
  amplicon
tctcagaaaccataatctaccatggctgatcctgcagaggaggaacaagaagatgaggaagaaatcgatgttgtttctgtggaaaagaggcaggctcctgg

SEQ ID NO: 1948 - DNA, 83 bp, Artificial Sequence
  amplicon
tctcagaaaccataatctaccatggctgatcctgcagctgaccatactccctggagccttctcccgaggtgcgcgggtgacct

SEQ ID NO: 1949 - DNA, 79 bp, Artificial Sequence
  amplicon
tctcagaaaccataatctaccatggctgatcctgcagtctgagcctgatggatttacagtgatcttcagtggtctgggg

SEQ ID NO: 1950 - DNA, 122 bp, Artificial Sequence
  amplicon
tctcagaaaccataatctaccatggctgatcctgcagctccgggcagagcgcgtgtggcggccgagcacatgggcccgcgggccgggcgggctcggggcggccgggacgaggaggggcgacg

SEQ ID NO: 1951 - DNA, 84 bp, Artificial Sequence
  amplicon
tacagtattggcaactaatacgttgggctgctcgcggccgccaccgccgggccccggccgtccctggctcccctcctgcctcga

SEQ ID NO: 1952 - DNA, 91 bp, Artificial Sequence
  amplicon
tacagtattggcaactaatacgttgggcagcctcccgcgacgatgcccctcaacgttagcttcaccaacaggaactatgacctcgactacg

SEQ ID NO: 1953 - DNA, 91 bp, Artificial Sequence
  amplicon
tacagtattggcaactaatacgttgggaggaggaacaagaagatgaggaagaaatcgatgttgtttctgtggaaaagaggcaggctcctgg

SEQ ID NO: 1954 - DNA, 73 bp, Artificial Sequence
  amplicon
tacagtattggcaactaatacgttgggctgaccatactccctggagccttctcccgaggtgcgcgggtgacct

SEQ ID NO: 1955 - DNA, 69 bp, Artificial Sequence
  amplicon
tacagtattggcaactaatacgttgggtctgagcctgatggatttacagtgatcttcagtggtctgggg

SEQ ID NO: 1956 - DNA, 112 bp, Artificial Sequence
  amplicon
tacagtattggcaactaatacgttgggctccgggcagagcgcgtgtggcggccgagcacatgggcccgcgggccgggcgggctcggggcggccgggacgaggaggggcgacg

SEQ ID NO: 1957 - DNA, 98 bp, Artificial Sequence
  amplicon
caaaagacggaaactctgtagtggtaacactacgcctataactgctcgcggccgccaccgccgggccccggccgtccctggctcccctcctgcctcga

SEQ ID NO: 1958 - DNA, 105 bp, Artificial Sequence
  amplicon
caaaagacggaaactctgtagtggtaacactacgcctataacagcctcccgcgacgatgcccctcaacgttagcttcaccaacaggaactatgacctcgactacg

SEQ ID NO: 1959 - DNA, 105 bp, Artificial Sequence
  amplicon
caaaagacggaaactctgtagtggtaacactacgcctataaaggaggaacaagaagatgaggaagaaatcgatgttgtttctgtggaaaagaggcaggctcctgg

SEQ ID NO: 1960 - DNA, 87 bp, Artificial Sequence
  amplicon
caaaagacggaaactctgtagtggtaacactacgcctataactgaccatactccctggagccttctcccgaggtgcgcgggtgacct

SEQ ID NO: 1961 - DNA, 83 bp, Artificial Sequence
  amplicon
caaaagacggaaactctgtagtggtaacactacgcctataatctgagcctgatggatttacagtgatcttcagtggtctgggg

SEQ ID NO: 1962 - DNA, 126 bp, Artificial Sequence
  amplicon
caaaagacggaaactctgtagtggtaacactacgcctataactccgggcagagcgcgtgtggcggccgagcacatgggcccgcgggccgggcgggctcggggcggccgggacgaggaggggcgacg

SEQ ID NO: 1963 - DNA, 75 bp, Artificial Sequence
  amplicon
caatggctgatccagaagctgctcgcggccgccaccgccgggccccggccgtccctggctcccctcctgcctcga

SEQ ID NO: 1964 - DNA, 82 bp, Artificial Sequence
  amplicon
caatggctgatccagaagcagcctcccgcgacgatgcccctcaacgttagcttcaccaacaggaactatgacctcgactacg

SEQ ID NO: 1965 - DNA, 82 bp, Artificial Sequence
  amplicon
caatggctgatccagaagaggaggaacaagaagatgaggaagaaatcgatgttgtttctgtggaaaagaggcaggctcctgg

SEQ ID NO: 1966 - DNA, 64 bp, Artificial Sequence
  amplicon
caatggctgatccagaagctgaccatactccctggagccttctcccgaggtgcgcgggtgacct

SEQ ID NO: 1967 - DNA, 60 bp, Artificial Sequence
  amplicon
caatggctgatccagaagtctgagcctgatggatttacagtgatcttcagtggtctgggg

SEQ ID NO: 1968 - DNA, 103 bp, Artificial Sequence
  amplicon
caatggctgatccagaagctccgggcagagcgcgtgtggcggccgagcacatgggcccgcgggccgggcgggctcggggcggccgggacgaggaggggcgacg

SEQ ID NO: 1969 - DNA, 130 bp, Artificial Sequence
  amplicon
atattagaacattatgaaaatgatagtaaacgactttgtgatcatatagactattggaaacatattcgacttgctgctcgcggccgccaccgccgggccccggccgtccctggctcccctcctgcctcga

SEQ ID NO: 1970 - DNA, 137 bp, Artificial Sequence
  amplicon
atattagaacattatgaaaatgatagtaaacgactttgtgatcatatagactattggaaacatattcgacttgcagcctcccgcgacgatgcccctcaacgttagcttcaccaacaggaactatgacctcgactacg

SEQ ID NO: 1971 - DNA, 137 bp, Artificial Sequence
  amplicon
atattagaacattatgaaaatgatagtaaacgactttgtgatcatatagactattggaaacatattcgacttgaggaggaacaagaagatgaggaagaaatcgatgttgtttctgtggaaaagaggcaggctcctgg

SEQ ID NO: 1972 - DNA, 119 bp, Artificial Sequence
  amplicon
atattagaacattatgaaaatgatagtaaacgactttgtgatcatatagactattggaaacatattcgacttgctgaccatactccctggagccttctcccgaggtgcgcgggtgacct

SEQ ID NO: 1973 - DNA, 115 bp, Artificial Sequence
  amplicon
atattagaacattatgaaaatgatagtaaacgactttgtgatcatatagactattggaaacatattcgacttgtctgagcctgatggatttacagtgatcttcagtggtctgggg

SEQ ID NO: 1974 - DNA, 158 bp, Artificial Sequence
  amplicon
atattagaacattatgaaaatgatagtaaacgactttgtgatcatatagactattggaaacatattcgacttgctccgggcagagcgcgtgtggcggccgagcacatgggcccgcgggccgggcgggctcggggcggccgggacgaggaggggcgacg

SEQ ID NO: 1975 - DNA, 95 bp, Artificial Sequence
  amplicon
aacaagggctgtcagttgtcctgcaactacacctataactgctcgcggccgccaccgccgggccccggccgtccctggctcccctcctgcctcga

SEQ ID NO: 1976 - DNA, 102 bp, Artificial Sequence
  amplicon
aacaagggctgtcagttgtcctgcaactacacctataacagcctcccgcgacgatgcccctcaacgttagcttcaccaacaggaactatgacctcgactacg

SEQ ID NO: 1977 - DNA, 102 bp, Artificial Sequence
  amplicon
aacaagggctgtcagttgtcctgcaactacacctataaaggaggaacaagaagatgaggaagaaatcgatgttgtttctgtggaaaagaggcaggctcctgg

SEQ ID NO: 1978 - DNA, 84 bp, Artificial Sequence
  amplicon
aacaagggctgtcagttgtcctgcaactacacctataactgaccatactccctggagccttctcccgaggtgcgcgggtgacct

SEQ ID NO: 1979 - DNA, 80 bp, Artificial Sequence
  amplicon
aacaagggctgtcagttgtcctgcaactacacctataatctgagcctgatggatttacagtgatcttcagtggtctgggg

SEQ ID NO: 1980 - DNA, 123 bp, Artificial Sequence
  amplicon
aacaagggctgtcagttgtcctgcaactacacctataactccgggcagagcgcgtgtggcggccgagcacatgggcccgcgggccgggcgggctcggggcggccgggacgaggaggggcgacg

SEQ ID NO: 1981 - DNA, 93 bp, Artificial Sequence
  amplicon
tctactagactgtaactacaatggctgatccagcagctgctcgcggccgccaccgccgggccccggccgtccctggctcccctcctgcctcga

SEQ ID NO: 1982 - DNA, 100 bp, Artificial Sequence
  amplicon
tctactagactgtaactacaatggctgatccagcagcagcctcccgcgacgatgcccctcaacgttagcttcaccaacaggaactatgacctcgactacg

SEQ ID NO: 1983 - DNA, 100 bp, Artificial Sequence
  amplicon
tctactagactgtaactacaatggctgatccagcagaggaggaacaagaagatgaggaagaaatcgatgttgtttctgtggaaaagaggcaggctcctgg

SEQ ID NO: 1984 - DNA, 82 bp, Artificial Sequence
  amplicon
tctactagactgtaactacaatggctgatccagcagctgaccatactccctggagccttctcccgaggtgcgcgggtgacct

SEQ ID NO: 1985 - DNA, 78 bp, Artificial Sequence
  amplicon
tctactagactgtaactacaatggctgatccagcagtctgagcctgatggatttacagtgatcttcagtggtctgggg

SEQ ID NO: 1986 - DNA, 121 bp, Artificial Sequence
  amplicon
tctactagactgtaactacaatggctgatccagcagctccgggcagagcgcgtgtggcggccgagcacatgggcccgcgggccgggcgggctcggggcggccgggacgaggaggggcgacg

SEQ ID NO: 1987 - DNA, 113 bp, Artificial Sequence
  amplicon
agctgataaaactgatttaccatcacaaattgaacattggaaactgatacgcatggctgctcgcggccgccaccgccgggccccggccgtccctggctcccctcctgcctcga

SEQ ID NO: 1988 - DNA, 120 bp, Artificial Sequence
  amplicon
agctgataaaactgatttaccatcacaaattgaacattggaaactgatacgcatggcagcctcccgcgacgatgcccctcaacgttagcttcaccaacaggaactatgacctcgactacg

SEQ ID NO: 1989 - DNA, 120 bp, Artificial Sequence
  amplicon
agctgataaaactgatttaccatcacaaattgaacattggaaactgatacgcatggaggaggaacaagaagatgaggaagaaatcgatgttgtttctgtggaaaagaggcaggctcctgg

SEQ ID NO: 1990 - DNA, 102 bp, Artificial Sequence
  amplicon
agctgataaaactgatttaccatcacaaattgaacattggaaactgatacgcatggctgaccatactccctggagccttctcccgaggtgcgcgggtgacct

SEQ ID NO: 1991 - DNA, 98 bp, Artificial Sequence
  amplicon
agctgataaaactgatttaccatcacaaattgaacattggaaactgatacgcatggtctgagcctgatggatttacagtgatcttcagtggtctgggg

SEQ ID NO: 1992 - DNA, 141 bp, Artificial Sequence
  amplicon
agctgataaaactgatttaccatcacaaattgaacattggaaactgatacgcatggctccgggcagagcgcgtgtggcggccgagcacatgggcccgcgggccgggcgggctcggggcggccgggacgaggaggggcgacg

SEQ ID NO: 1993 - DNA, 99 bp, Artificial Sequence
  amplicon
acaagcagcggactgtgtgtagttctaacgttgcacctatagctgctcgcggccgccaccgccgggccccggccgtccctggctcccctcctgcctcga

SEQ ID NO: 1994 - DNA, 106 bp, Artificial Sequence
  amplicon
acaagcagcggactgtgtgtagttctaacgttgcacctatagcagcctcccgcgacgatgcccctcaacgttagcttcaccaacaggaactatgacctcgactacg

SEQ ID NO: 1995 - DNA, 106 bp, Artificial Sequence
  amplicon
acaagcagcggactgtgtgtagttctaacgttgcacctatagaggaggaacaagaagatgaggaagaaatcgatgttgtttctgtggaaaagaggcaggctcctgg

SEQ ID NO: 1996 - DNA, 88 bp, Artificial Sequence
  amplicon
acaagcagcggactgtgtgtagttctaacgttgcacctatagctgaccatactccctggagccttctcccgaggtgcgcgggtgacct

SEQ ID NO: 1997 - DNA, 84 bp, Artificial Sequence
  amplicon
acaagcagcggactgtgtgtagttctaacgttgcacctatagtctgagcctgatggatttacagtgatcttcagtggtctgggg

SEQ ID NO: 1998 - DNA, 127 bp, Artificial Sequence
  amplicon
acaagcagcggactgtgtgtagttctaacgttgcacctatagctccgggcagagcgcgtgtggcggccgagcacatgggcccgcgggccgggcgggctcggggcggccgggacgaggaggggcgacg

SEQ ID NO: 1999 - DNA, 91 bp, Artificial Sequence
  amplicon
caataaacatcatctacaatggccgatcctgaagctgctcgcggccgccaccgccgggccccggccgtccctggctcccctcctgcctcga

SEQ ID NO: 2000 - DNA, 98 bp, Artificial Sequence
  amplicon
caataaacatcatctacaatggccgatcctgaagcagcctcccgcgacgatgcccctcaacgttagcttcaccaacaggaactatgacctcgactacg

SEQ ID NO: 2001 - DNA, 98 bp, Artificial Sequence
  amplicon
caataaacatcatctacaatggccgatcctgaagaggaggaacaagaagatgaggaagaaatcgatgttgtttctgtggaaaagaggcaggctcctgg

SEQ ID NO: 2002 - DNA, 80 bp, Artificial Sequence
  amplicon
caataaacatcatctacaatggccgatcctgaagctgaccatactccctggagccttctcccgaggtgcgcgggtgacct

SEQ ID NO: 2003 - DNA, 76 bp, Artificial Sequence
  amplicon
caataaacatcatctacaatggccgatcctgaagtctgagcctgatggatttacagtgatcttcagtggtctgggg

SEQ ID NO: 2004 - DNA, 119 bp, Artificial Sequence
  amplicon
caataaacatcatctacaatggccgatcctgaagctccgggcagagcgcgtgtggcggccgagcacatgggcccgcgggccgggcgggctcggggcggccgggacgaggaggggcgacg

SEQ ID NO: 2005 - DNA, 93 bp, Artificial Sequence
  amplicon
ctgatcacatacagtattggaaactgattcgtcttgctgctcgcggccgccaccgccgggccccggccgtccctggctcccctcctgcctcga

SEQ ID NO: 2006 - DNA, 100 bp, Artificial Sequence
  amplicon
ctgatcacatacagtattggaaactgattcgtcttgcagcctcccgcgacgatgcccctcaacgttagcttcaccaacaggaactatgacctcgactacg

SEQ ID NO: 2007 - DNA, 100 bp, Artificial Sequence
  amplicon
ctgatcacatacagtattggaaactgattcgtcttgaggaggaacaagaagatgaggaagaaatcgatgttgtttctgtggaaaagaggcaggctcctgg

SEQ ID NO: 2008 - DNA, 82 bp, Artificial Sequence
  amplicon
ctgatcacatacagtattggaaactgattcgtcttgctgaccatactccctggagccttctcccgaggtgcgcgggtgacct

SEQ ID NO: 2009 - DNA, 78 bp, Artificial Sequence
  amplicon
ctgatcacatacagtattggaaactgattcgtcttgtctgagcctgatggatttacagtgatcttcagtggtctgggg

SEQ ID NO: 2010 - DNA, 121 bp, Artificial Sequence
  amplicon
ctgatcacatacagtattggaaactgattcgtcttgctccgggcagagcgcgtgtggcggccgagcacatgggcccgcgggccgggcgggctcggggcggccgggacgaggaggggcgacg

SEQ ID NO: 2011 - DNA, 92 bp, Artificial Sequence
  amplicon
ccggtgtggtagttgtagtacaactacacctatagctgctcgcggccgccaccgccgggccccggccgtccctggctcccctcctgcctcga

SEQ ID NO: 2012 - DNA, 99 bp, Artificial Sequence
  amplicon
ccggtgtggtagttgtagtacaactacacctatagcagcctcccgcgacgatgcccctcaacgttagcttcaccaacaggaactatgacctcgactacg

SEQ ID NO: 2013 - DNA, 99 bp, Artificial Sequence
  amplicon
ccggtgtggtagttgtagtacaactacacctatagaggaggaacaagaagatgaggaagaaatcgatgttgtttctgtggaaaagaggcaggctcctgg

SEQ ID NO: 2014 - DNA, 81 bp, Artificial Sequence
  amplicon
ccggtgtggtagttgtagtacaactacacctatagctgaccatactccctggagccttctcccgaggtgcgcgggtgacct

SEQ ID NO: 2015 - DNA, 77 bp, Artificial Sequence
  amplicon
ccggtgtggtagttgtagtacaactacacctatagtctgagcctgatggatttacagtgatcttcagtggtctgggg

SEQ ID NO: 2016 - DNA, 120 bp, Artificial Sequence
  amplicon
ccggtgtggtagttgtagtacaactacacctatagctccgggcagagcgcgtgtggcggccgagcacatgggcccgcgggccgggcgggctcggggcggccgggacgaggaggggcgacg

SEQ ID NO: 2017 - DNA, 78 bp, Artificial Sequence
  amplicon
ctacaatggctgatcctgcagctgctcgcggccgccaccgccgggccccggccgtccctggctcccctcctgcctcga

SEQ ID NO: 2018 - DNA, 85 bp, Artificial Sequence
  amplicon
ctacaatggctgatcctgcagcagcctcccgcgacgatgcccctcaacgttagcttcaccaacaggaactatgacctcgactacg

SEQ ID NO: 2019 - DNA, 85 bp, Artificial Sequence
  amplicon
ctacaatggctgatcctgcagaggaggaacaagaagatgaggaagaaatcgatgttgtttctgtggaaaagaggcaggctcctgg

SEQ ID NO: 2020 - DNA, 67 bp, Artificial Sequence
  amplicon
ctacaatggctgatcctgcagctgaccatactccctggagccttctcccgaggtgcgcgggtgacct

SEQ ID NO: 2021 - DNA, 63 bp, Artificial Sequence
  amplicon
ctacaatggctgatcctgcagtctgagcctgatggatttacagtgatcttcagtggtctgggg

SEQ ID NO: 2022 - DNA, 106 bp, Artificial Sequence
  amplicon
ctacaatggctgatcctgcagctccgggcagagcgcgtgtggcggccgagcacatgggcccgcgggccgggcgggctcggggcggccgggacgaggaggggcgacg

SEQ ID NO: 2023 - DNA, 132 bp, Artificial Sequence
  amplicon
aaatactagaatactatgaacaagacagtaaatcaatatatgatcaaattaattattggaaatgtgtgcgaatggctgctcgcggccgccaccgccgggccccggccgtccctggctcccctcctgcctcga

SEQ ID NO: 2024 - DNA, 139 bp, Artificial Sequence
  amplicon
aaatactagaatactatgaacaagacagtaaatcaatatatgatcaaattaattattggaaatgtgtgcgaatggcagcctcccgcgacgatgcccctcaacgttagcttcaccaacaggaactatgacctcgactacg

SEQ ID NO: 2025 - DNA, 139 bp, Artificial Sequence
  amplicon
aaatactagaatactatgaacaagacagtaaatcaatatatgatcaaattaattattggaaatgtgtgcgaatggaggaggaacaagaagatgaggaagaaatcgatgttgtttctgtggaaaagaggcaggctcctgg

SEQ ID NO: 2026 - DNA, 121 bp, Artificial Sequence
  amplicon
aaatactagaatactatgaacaagacagtaaatcaatatatgatcaaattaattattggaaatgtgtgcgaatggctgaccatactccctggagccttctcccgaggtgcgcgggtgacct

SEQ ID NO: 2027 - DNA, 117 bp, Artificial Sequence
  amplicon
aaatactagaatactatgaacaagacagtaaatcaatatatgatcaaattaattattggaaatgtgtgcgaatggtctgagcctgatggatttacagtgatcttcagtggtctgggg

SEQ ID NO: 2028 - DNA, 160 bp, Artificial Sequence
  amplicon
aaatactagaatactatgaacaagacagtaaatcaatatatgatcaaattaattattggaaatgtgtgcgaatggctccgggcagagcgcgtgtggcggccgagcacatgggcccgcgggccgggcgggctcggggcggccgggacgaggaggggcgacg

SEQ ID NO: 2029 - DNA, 99 bp, Artificial Sequence
  amplicon
acacaagacggtacctcagttgtggtaacactacgcctataactgctcgcggccgccaccgccgggccccggccgtccctggctcccctcctgcctcga

SEQ ID NO: 2030 - DNA, 106 bp, Artificial Sequence
  amplicon
acacaagacggtacctcagttgtggtaacactacgcctataacagcctcccgcgacgatgcccctcaacgttagcttcaccaacaggaactatgacctcgactacg

SEQ ID NO: 2031 - DNA, 106 bp, Artificial Sequence
  amplicon
acacaagacggtacctcagttgtggtaacactacgcctataaaggaggaacaagaagatgaggaagaaatcgatgttgtttctgtggaaaagaggcaggctcctgg

SEQ ID NO: 2032 - DNA, 88 bp, Artificial Sequence
  amplicon
acacaagacggtacctcagttgtggtaacactacgcctataactgaccatactccctggagccttctcccgaggtgcgcgggtgacct

SEQ ID NO: 2033 - DNA, 84 bp, Artificial Sequence
  amplicon
acacaagacggtacctcagttgtggtaacactacgcctataatctgagcctgatggatttacagtgatcttcagtggtctgggg

SEQ ID NO: 2034 - DNA, 127 bp, Artificial Sequence
  amplicon
acacaagacggtacctcagttgtggtaacactacgcctataactccgggcagagcgcgtgtggcggccgagcacatgggcccgcgggccgggcgggctcggggcggccgggacgaggaggggcgacg

SEQ ID NO: 2035 - DNA, 87 bp, Artificial Sequence
  amplicon
accagtaacctgctatggccaatcgtgaagctgctcgcggccgccaccgccgggccccggccgtccctggctcccctcctgcctcga

SEQ ID NO: 2036 - DNA, 94 bp, Artificial Sequence
  amplicon
accagtaacctgctatggccaatcgtgaagcagcctcccgcgacgatgcccctcaacgttagcttcaccaacaggaactatgacctcgactacg

SEQ ID NO: 2037 - DNA, 94 bp, Artificial Sequence
  amplicon
accagtaacctgctatggccaatcgtgaagaggaggaacaagaagatgaggaagaaatcgatgttgtttctgtggaaaagaggcaggctcctgg

SEQ ID NO: 2038 - DNA, 76 bp, Artificial Sequence
  amplicon
accagtaacctgctatggccaatcgtgaagctgaccatactccctggagccttctcccgaggtgcgcgggtgacct

SEQ ID NO: 2039 - DNA, 72 bp, Artificial Sequence
  amplicon
accagtaacctgctatggccaatcgtgaagtctgagcctgatggatttacagtgatcttcagtggtctgggg

SEQ ID NO: 2040 - DNA, 115 bp, Artificial Sequence
  amplicon
accagtaacctgctatggccaatcgtgaagctccgggcagagcgcgtgtggcggccgagcacatgggcccgcgggccgggcgggctcggggcggccgggacgaggaggggcgacg

SEQ ID NO: 2041 - DNA, 116 bp, Artificial Sequence
  amplicon
tgaaaatgacagtaaagacataaacagccaaataagttattggcaacttatacgtttggctgctcgcggccgccaccgccgggccccggccgtccctggctcccctcctgcctcga

SEQ ID NO: 2042 - DNA, 123 bp, Artificial Sequence
  amplicon
tgaaaatgacagtaaagacataaacagccaaataagttattggcaacttatacgtttggcagcctcccgcgacgatgcccctcaacgttagcttcaccaacaggaactatgacctcgactacg

SEQ ID NO: 2043 - DNA, 123 bp, Artificial Sequence
  amplicon
tgaaaatgacagtaaagacataaacagccaaataagttattggcaacttatacgtttggaggaggaacaagaagatgaggaagaaatcgatgttgtttctgtggaaaagaggcaggctcctgg

SEQ ID NO: 2044 - DNA, 105 bp, Artificial Sequence
  amplicon
tgaaaatgacagtaaagacataaacagccaaataagttattggcaacttatacgtttggctgaccatactccctggagccttctcccgaggtgcgcgggtgacct

SEQ ID NO: 2045 - DNA, 101 bp, Artificial Sequence
  amplicon
tgaaaatgacagtaaagacataaacagccaaataagttattggcaacttatacgtttggtctgagcctgatggatttacagtgatcttcagtggtctgggg

SEQ ID NO: 2046 - DNA, 144 bp, Artificial Sequence
  amplicon
tgaaaatgacagtaaagacataaacagccaaataagttattggcaacttatacgtttggctccgggcagagcgcgtgtggcggccgagcacatgggcccgcgggccgggcgggctcggggcggccgggacgaggaggggcgacg

SEQ ID NO: 2047 - DNA, 95 bp, Artificial Sequence
  amplicon
aagaaggaaagtgtgtagtggtaacactacgcctataactgctcgcggccgccaccgccgggccccggccgtccctggctcccctcctgcctcga

SEQ ID NO: 2048 - DNA, 102 bp, Artificial Sequence
  amplicon
aagaaggaaagtgtgtagtggtaacactacgcctataacagcctcccgcgacgatgcccctcaacgttagcttcaccaacaggaactatgacctcgactacg

SEQ ID NO: 2049 - DNA, 102 bp, Artificial Sequence
  amplicon
aagaaggaaagtgtgtagtggtaacactacgcctataaaggaggaacaagaagatgaggaagaaatcgatgttgtttctgtggaaaagaggcaggctcctgg

SEQ ID NO: 2050 - DNA, 84 bp, Artificial Sequence
  amplicon
aagaaggaaagtgtgtagtggtaacactacgcctataactgaccatactccctggagccttctcccgaggtgcgcgggtgacct

SEQ ID NO: 2051 - DNA, 80 bp, Artificial Sequence
  amplicon
aagaaggaaagtgtgtagtggtaacactacgcctataatctgagcctgatggatttacagtgatcttcagtggtctgggg

SEQ ID NO: 2052 - DNA, 123 bp, Artificial Sequence
  amplicon
aagaaggaaagtgtgtagtggtaacactacgcctataactccgggcagagcgcgtgtggcggccgagcacatgggcccgcgggccgggcgggctcggggcggccgggacgaggaggggcgacg

SEQ ID NO: 2053 - DNA, 103 bp, Artificial Sequence
  amplicon
ccgtggtgtgcaactaaccaataatctacaatggcggatccagaagctgctcgcggccgccaccgccgggccccggccgtccctggctcccctcctgcctcga

SEQ ID NO: 2054 - DNA, 110 bp, Artificial Sequence
  amplicon
ccgtggtgtgcaactaaccaataatctacaatggcggatccagaagcagcctcccgcgacgatgcccctcaacgttagcttcaccaacaggaactatgacctcgactacg

SEQ ID NO: 2055 - DNA, 110 bp, Artificial Sequence
  amplicon
ccgtggtgtgcaactaaccaataatctacaatggcggatccagaagaggaggaacaagaagatgaggaagaaatcgatgttgtttctgtggaaaagaggcaggctcctgg

SEQ ID NO: 2056 - DNA, 92 bp, Artificial Sequence
  amplicon
ccgtggtgtgcaactaaccaataatctacaatggcggatccagaagctgaccatactccctggagccttctcccgaggtgcgcgggtgacct

SEQ ID NO: 2057 - DNA, 88 bp, Artificial Sequence
  amplicon
ccgtggtgtgcaactaaccaataatctacaatggcggatccagaagtctgagcctgatggatttacagtgatcttcagtggtctgggg

SEQ ID NO: 2058 - DNA, 131 bp, Artificial Sequence
  amplicon
ccgtggtgtgcaactaaccaataatctacaatggcggatccagaagctccgggcagagcgcgtgtggcggccgagcacatgggcccgcgggccgggcgggctcggggcggccgggacgaggaggggcgacg

SEQ ID NO: 2059 - DNA, 115 bp, Artificial Sequence
  amplicon
gaactggacagtgataaattagtagatcaaattaactattggacattgttacgatatgctgctcgcggccgccaccgccgggccccggccgtccctggctcccctcctgcctcga

SEQ ID NO: 2060 - DNA, 122 bp, Artificial Sequence
  amplicon
gaactggacagtgataaattagtagatcaaattaactattggacattgttacgatatgcagcctcccgcgacgatgcccctcaacgttagcttcaccaacaggaactatgacctcgactacg

SEQ ID NO: 2061 - DNA, 122 bp, Artificial Sequence
  amplicon
gaactggacagtgataaattagtagatcaaattaactattggacattgttacgatatgaggaggaacaagaagatgaggaagaaatcgatgttgtttctgtggaaaagaggcaggctcctgg

SEQ ID NO: 2062 - DNA, 104 bp, Artificial Sequence
  amplicon
gaactggacagtgataaattagtagatcaaattaactattggacattgttacgatatgctgaccatactccctggagccttctcccgaggtgcgcgggtgacct

SEQ ID NO: 2063 - DNA, 100 bp, Artificial Sequence
  amplicon
gaactggacagtgataaattagtagatcaaattaactattggacattgttacgatatgtctgagcctgatggatttacagtgatcttcagtggtctgggg

SEQ ID NO: 2064 - DNA, 143 bp, Artificial Sequence
  amplicon
gaactggacagtgataaattagtagatcaaattaactattggacattgttacgatatgctccgggcagagcgcgtgtggcggccgagcacatgggcccgcgggccgggcgggctcggggcggccgggacgaggaggggcgacg

SEQ ID NO: 2065 - DNA, 81 bp, Artificial Sequence
  amplicon
gtgcaactcagactgcgtttatagctgctcgcggccgccaccgccgggccccggccgtccctggctcccctcctgcctcga

SEQ ID NO: 2066 - DNA, 88 bp, Artificial Sequence
  amplicon
gtgcaactcagactgcgtttatagcagcctcccgcgacgatgcccctcaacgttagcttcaccaacaggaactatgacctcgactacg

SEQ ID NO: 2067 - DNA, 88 bp, Artificial Sequence
  amplicon
gtgcaactcagactgcgtttatagaggaggaacaagaagatgaggaagaaatcgatgttgtttctgtggaaaagaggcaggctcctgg

SEQ ID NO: 2068 - DNA, 70 bp, Artificial Sequence
  amplicon
gtgcaactcagactgcgtttatagctgaccatactccctggagccttctcccgaggtgcgcgggtgacct

SEQ ID NO: 2069 - DNA, 66 bp, Artificial Sequence
  amplicon
gtgcaactcagactgcgtttatagtctgagcctgatggatttacagtgatcttcagtggtctgggg

SEQ ID NO: 2070 - DNA, 109 bp, Artificial Sequence
  amplicon
gtgcaactcagactgcgtttatagctccgggcagagcgcgtgtggcggccgagcacatgggcccgcgggccgggcgggctcggggcggccgggacgaggaggggcgacg

SEQ ID NO: 2071 - DNA, 101 bp, Artificial Sequence
  amplicon
gcccgtgttgtgcgaacaactagcaacggcgatggactgtgaagctgctcgcggccgccaccgccgggccccggccgtccctggctcccctcctgcctcga

SEQ ID NO: 2072 - DNA, 108 bp, Artificial Sequence
  amplicon
gcccgtgttgtgcgaacaactagcaacggcgatggactgtgaagcagcctcccgcgacgatgcccctcaacgttagcttcaccaacaggaactatgacctcgactacg

SEQ ID NO: 2073 - DNA, 108 bp, Artificial Sequence
  amplicon
gcccgtgttgtgcgaacaactagcaacggcgatggactgtgaagaggaggaacaagaagatgaggaagaaatcgatgttgtttctgtggaaaagaggcaggctcctgg

SEQ ID NO: 2074 - DNA, 90 bp, Artificial Sequence
  amplicon
gcccgtgttgtgcgaacaactagcaacggcgatggactgtgaagctgaccatactccctggagccttctcccgaggtgcgcgggtgacct

SEQ ID NO: 2075 - DNA, 86 bp, Artificial Sequence
  amplicon
gcccgtgttgtgcgaacaactagcaacggcgatggactgtgaagtctgagcctgatggatttacagtgatcttcagtggtctgggg

SEQ ID NO: 2076 - DNA, 129 bp, Artificial Sequence
  amplicon
gcccgtgttgtgcgaacaactagcaacggcgatggactgtgaagctccgggcagagcgcgtgtggcggccgagcacatgggcccgcgggccgggcgggctcggggcggccgggacgaggaggggcgacg

SEQ ID NO: 2077 - DNA, 84 bp, Artificial Sequence
  amplicon
ttgaacattggaaattgactcgaatggctgctcgcggccgccaccgccgggccccggccgtccctggctcccctcctgcctcga

SEQ ID NO: 2078 - DNA, 91 bp, Artificial Sequence
  amplicon
ttgaacattggaaattgactcgaatggcagcctcccgcgacgatgcccctcaacgttagcttcaccaacaggaactatgacctcgactacg

SEQ ID NO: 2079 - DNA, 91 bp, Artificial Sequence
  amplicon
ttgaacattggaaattgactcgaatggaggaggaacaagaagatgaggaagaaatcgatgttgtttctgtggaaaagaggcaggctcctgg

SEQ ID NO: 2080 - DNA, 73 bp, Artificial Sequence
  amplicon
ttgaacattggaaattgactcgaatggctgaccatactccctggagccttctcccgaggtgcgcgggtgacct

SEQ ID NO: 2081 - DNA, 69 bp, Artificial Sequence
  amplicon
ttgaacattggaaattgactcgaatggtctgagcctgatggatttacagtgatcttcagtggtctgggg

SEQ ID NO: 2082 - DNA, 112 bp, Artificial Sequence
  amplicon
ttgaacattggaaattgactcgaatggctccgggcagagcgcgtgtggcggccgagcacatgggcccgcgggccgggcgggctcggggcggccgggacgaggaggggcgacg

SEQ ID NO: 2083 - DNA, 99 bp, Artificial Sequence
  amplicon
acaaaggacgggttgcacatacaacttgtactgcacctataactgctcgcggccgccaccgccgggccccggccgtccctggctcccctcctgcctcga

SEQ ID NO: 2084 - DNA, 106 bp, Artificial Sequence
  amplicon
acaaaggacgggttgcacatacaacttgtactgcacctataacagcctcccgcgacgatgcccctcaacgttagcttcaccaacaggaactatgacctcgactacg

SEQ ID NO: 2085 - DNA, 106 bp, Artificial Sequence
  amplicon
acaaaggacgggttgcacatacaacttgtactgcacctataaaggaggaacaagaagatgaggaagaaatcgatgttgtttctgtggaaaagaggcaggctcctgg

SEQ ID NO: 2086 - DNA, 88 bp, Artificial Sequence
  amplicon
acaaaggacgggttgcacatacaacttgtactgcacctataactgaccatactccctggagccttctcccgaggtgcgcgggtgacct

SEQ ID NO: 2087 - DNA, 84 bp, Artificial Sequence
  amplicon
acaaaggacgggttgcacatacaacttgtactgcacctataatctgagcctgatggatttacagtgatcttcagtggtctgggg

SEQ ID NO: 2088 - DNA, 127 bp, Artificial Sequence
  amplicon
acaaaggacgggttgcacatacaacttgtactgcacctataactccgggcagagcgcgtgtggcggccgagcacatgggcccgcgggccgggcgggctcggggcggccgggacgaggaggggcgacg

SEQ ID NO: 2089 - DNA, 111 bp, Artificial Sequence
  amplicon
gtgtgccccggctgtgcacggctataaacaaccctgcaatggaggaccctgaagctgctcgcggccgccaccgccgggccccggccgtccctggctcccctcctgcctcga

SEQ ID NO: 2090 - DNA, 118 bp, Artificial Sequence
  amplicon
gtgtgccccggctgtgcacggctataaacaaccctgcaatggaggaccctgaagcagcctcccgcgacgatgcccctcaacgttagcttcaccaacaggaactatgacctcgactacg

SEQ ID NO: 2091 - DNA, 118 bp, Artificial Sequence
  amplicon
gtgtgccccggctgtgcacggctataaacaaccctgcaatggaggaccctgaagaggaggaacaagaagatgaggaagaaatcgatgttgtttctgtggaaaagaggcaggctcctgg

SEQ ID NO: 2092 - DNA, 100 bp, Artificial Sequence
  amplicon
gtgtgccccggctgtgcacggctataaacaaccctgcaatggaggaccctgaagctgaccatactccctggagccttctcccgaggtgcgcgggtgacct

SEQ ID NO: 2093 - DNA, 96 bp, Artificial Sequence
  amplicon
gtgtgccccggctgtgcacggctataaacaaccctgcaatggaggaccctgaagtctgagcctgatggatttacagtgatcttcagtggtctgggg

SEQ ID NO: 2094 - DNA, 139 bp, Artificial Sequence
  amplicon
gtgtgccccggctgtgcacggctataaacaaccctgcaatggaggaccctgaagctccgggcagagcgcgtgtggcggccgagcacatgggcccgcgggccgggcgggctcggggcggccgggacgaggaggggcgacg

SEQ ID NO: 2095 - DNA, 117 bp, Artificial Sequence
  amplicon
tgaaaaaagatagtagatgtattgcagatcatatagaatattggaaagctgtgcgacatgctgctcgcggccgccaccgccgggccccggccgtccctggctcccctcctgcctcga

SEQ ID NO: 2096 - DNA, 124 bp, Artificial Sequence
  amplicon
tgaaaaaagatagtagatgtattgcagatcatatagaatattggaaagctgtgcgacatgcagcctcccgcgacgatgcccctcaacgttagcttcaccaacaggaactatgacctcgactacg

SEQ ID NO: 2097 - DNA, 124 bp, Artificial Sequence
  amplicon
tgaaaaaagatagtagatgtattgcagatcatatagaatattggaaagctgtgcgacatgaggaggaacaagaagatgaggaagaaatcgatgttgtttctgtggaaaagaggcaggctcctgg

SEQ ID NO: 2098 - DNA, 106 bp, Artificial Sequence
  amplicon
tgaaaaaagatagtagatgtattgcagatcatatagaatattggaaagctgtgcgacatgctgaccatactccctggagccttctcccgaggtgcgcgggtgacct

SEQ ID NO: 2099 - DNA, 102 bp, Artificial Sequence
  amplicon
tgaaaaaagatagtagatgtattgcagatcatatagaatattggaaagctgtgcgacatgtctgagcctgatggatttacagtgatcttcagtggtctgggg

SEQ ID NO: 2100 - DNA, 145 bp, Artificial Sequence
  amplicon
tgaaaaaagatagtagatgtattgcagatcatatagaatattggaaagctgtgcgacatgctccgggcagagcgcgtgtggcggccgagcacatgggcccgcgggccgggcgggctcggggcggccgggacgaggaggggcgacg

SEQ ID NO: 2101 - DNA, 70 bp, Artificial Sequence
  amplicon
actacgcctgtagctgctcgcggccgccaccgccgggccccggccgtccctggctcccctcctgcctcga

SEQ ID NO: 2102 - DNA, 77 bp, Artificial Sequence
  amplicon
actacgcctgtagcagcctcccgcgacgatgcccctcaacgttagcttcaccaacaggaactatgacctcgactacg

SEQ ID NO: 2103 - DNA, 77 bp, Artificial Sequence
  amplicon
actacgcctgtagaggaggaacaagaagatgaggaagaaatcgatgttgtttctgtggaaaagaggcaggctcctgg

SEQ ID NO: 2104 - DNA, 59 bp, Artificial Sequence
  amplicon
actacgcctgtagctgaccatactccctggagccttctcccgaggtgcgcgggtgacct

SEQ ID NO: 2105 - DNA, 55 bp, Artificial Sequence
  amplicon
actacgcctgtagtctgagcctgatggatttacagtgatcttcagtggtctgggg

SEQ ID NO: 2106 - DNA, 98 bp, Artificial Sequence
  amplicon
actacgcctgtagctccgggcagagcgcgtgtggcggccgagcacatgggcccgcgggccgggcgggctcggggcggccgggacgaggaggggcgacg

SEQ ID NO: 2107 - DNA, 95 bp, Artificial Sequence
  amplicon
gcgcatcaagtaactaactgcaatggcgtcacctgaagctgctcgcggccgccaccgccgggccccggccgtccctggctcccctcctgcctcga

SEQ ID NO: 2108 - DNA, 102 bp, Artificial Sequence
  amplicon
gcgcatcaagtaactaactgcaatggcgtcacctgaagcagcctcccgcgacgatgcccctcaacgttagcttcaccaacaggaactatgacctcgactacg

SEQ ID NO: 2109 - DNA, 102 bp, Artificial Sequence
  amplicon
gcgcatcaagtaactaactgcaatggcgtcacctgaagaggaggaacaagaagatgaggaagaaatcgatgttgtttctgtggaaaagaggcaggctcctgg

SEQ ID NO: 2110 - DNA, 84 bp, Artificial Sequence
  amplicon
gcgcatcaagtaactaactgcaatggcgtcacctgaagctgaccatactccctggagccttctcccgaggtgcgcgggtgacct

SEQ ID NO: 2111 - DNA, 80 bp, Artificial Sequence
  amplicon
gcgcatcaagtaactaactgcaatggcgtcacctgaagtctgagcctgatggatttacagtgatcttcagtggtctgggg

SEQ ID NO: 2112 - DNA, 123 bp, Artificial Sequence
  amplicon
gcgcatcaagtaactaactgcaatggcgtcacctgaagctccgggcagagcgcgtgtggcggccgagcacatgggcccgcgggccgggcgggctcggggcggccgggacgaggaggggcgacg

SEQ ID NO: 2113 - DNA, 112 bp, Artificial Sequence
  amplicon
gctgataaaaatgatttaacatcacaaattgaacattggaaactaatacgcatggctgctcgcggccgccaccgccgggccccggccgtccctggctcccctcctgcctcga

SEQ ID NO: 2114 - DNA, 119 bp, Artificial Sequence
  amplicon
gctgataaaaatgatttaacatcacaaattgaacattggaaactaatacgcatggcagcctcccgcgacgatgcccctcaacgttagcttcaccaacaggaactatgacctcgactacg

SEQ ID NO: 2115 - DNA, 119 bp, Artificial Sequence
  amplicon
gctgataaaaatgatttaacatcacaaattgaacattggaaactaatacgcatggaggaggaacaagaagatgaggaagaaatcgatgttgtttctgtggaaaagaggcaggctcctgg

SEQ ID NO: 2116 - DNA, 101 bp, Artificial Sequence
  amplicon
gctgataaaaatgatttaacatcacaaattgaacattggaaactaatacgcatggctgaccatactccctggagccttctcccgaggtgcgcgggtgacct

SEQ ID NO: 2117 - DNA, 97 bp, Artificial Sequence
  amplicon
gctgataaaaatgatttaacatcacaaattgaacattggaaactaatacgcatggtctgagcctgatggatttacagtgatcttcagtggtctgggg

SEQ ID NO: 2118 - DNA, 140 bp, Artificial Sequence
  amplicon
gctgataaaaatgatttaacatcacaaattgaacattggaaactaatacgcatggctccgggcagagcgcgtgtggcggccgagcacatgggcccgcgggccgggcgggctcggggcggccgggacgaggaggggcgacg

SEQ ID NO: 2119 - DNA, 105 bp, Artificial Sequence
  amplicon
gtacatacaaagggcggaacgtgtgtagttctaaagtttcacctatcgctgctcgcggccgccaccgccgggccccggccgtccctggctcccctcctgcctcga

SEQ ID NO: 2120 - DNA, 112 bp, Artificial Sequence
  amplicon
gtacatacaaagggcggaacgtgtgtagttctaaagtttcacctatcgcagcctcccgcgacgatgcccctcaacgttagcttcaccaacaggaactatgacctcgactacg

SEQ ID NO: 2121 - DNA, 112 bp, Artificial Sequence
  amplicon
gtacatacaaagggcggaacgtgtgtagttctaaagtttcacctatcgaggaggaacaagaagatgaggaagaaatcgatgttgtttctgtggaaaagaggcaggctcctgg

SEQ ID NO: 2122 - DNA, 94 bp, Artificial Sequence
  amplicon
gtacatacaaagggcggaacgtgtgtagttctaaagtttcacctatcgctgaccatactccctggagccttctcccgaggtgcgcgggtgacct

SEQ ID NO: 2123 - DNA, 90 bp, Artificial Sequence
  amplicon
gtacatacaaagggcggaacgtgtgtagttctaaagtttcacctatcgtctgagcctgatggatttacagtgatcttcagtggtctgggg

SEQ ID NO: 2124 - DNA, 133 bp, Artificial Sequence
  amplicon
gtacatacaaagggcggaacgtgtgtagttctaaagtttcacctatcgctccgggcagagcgcgtgtggcggccgagcacatgggcccgcgggccgggcgggctcggggcggccgggacgaggaggggcgacg

SEQ ID NO: 2125 - DNA, 112 bp, Artificial Sequence
  amplicon
gtgtgccctagctgtgcacagcaataaacaccatctgcaatggatgaccctgaagctgctcgcggccgccaccgccgggccccggccgtccctggctcccctcctgcctcga

SEQ ID NO: 2126 - DNA, 119 bp, Artificial Sequence
  amplicon
gtgtgccctagctgtgcacagcaataaacaccatctgcaatggatgaccctgaagcagcctcccgcgacgatgcccctcaacgttagcttcaccaacaggaactatgacctcgactacg

SEQ ID NO: 2127 - DNA, 119 bp, Artificial Sequence
  amplicon
gtgtgccctagctgtgcacagcaataaacaccatctgcaatggatgaccctgaagaggaggaacaagaagatgaggaagaaatcgatgttgtttctgtggaaaagaggcaggctcctgg

SEQ ID NO: 2128 - DNA, 101 bp, Artificial Sequence
  amplicon
gtgtgccctagctgtgcacagcaataaacaccatctgcaatggatgaccctgaagctgaccatactccctggagccttctcccgaggtgcgcgggtgacct

SEQ ID NO: 2129 - DNA, 97 bp, Artificial Sequence
  amplicon
gtgtgccctagctgtgcacagcaataaacaccatctgcaatggatgaccctgaagtctgagcctgatggatttacagtgatcttcagtggtctgggg

SEQ ID NO: 2130 - DNA, 140 bp, Artificial Sequence
  amplicon
gtgtgccctagctgtgcacagcaataaacaccatctgcaatggatgaccctgaagctccgggcagagcgcgtgtggcggccgagcacatgggcccgcgggccgggcgggctcggggcggccgggacgaggaggggcgacg

SEQ ID NO: 2131 - DNA, 128 bp, Artificial Sequence
  amplicon
attagaacattatgaaaacgatagtaaagacattaatgaacacataaactattggaaactggtgcgtatggctgctcgcggccgccaccgccgggccccggccgtccctggctcccctcctgcctcga

SEQ ID NO: 2132 - DNA, 135 bp, Artificial Sequence
  amplicon
attagaacattatgaaaacgatagtaaagacattaatgaacacataaactattggaaactggtgcgtatggcagcctcccgcgacgatgcccctcaacgttagcttcaccaacaggaactatgacctcgactacg

SEQ ID NO: 2133 - DNA, 135 bp, Artificial Sequence
  amplicon
attagaacattatgaaaacgatagtaaagacattaatgaacacataaactattggaaactggtgcgtatggaggaggaacaagaagatgaggaagaaatcgatgttgtttctgtggaaaagaggcaggctcctgg

SEQ ID NO: 2134 - DNA, 117 bp, Artificial Sequence
  amplicon
attagaacattatgaaaacgatagtaaagacattaatgaacacataaactattggaaactggtgcgtatggctgaccatactccctggagccttctcccgaggtgcgcgggtgacct

SEQ ID NO: 2135 - DNA, 113 bp, Artificial Sequence
  amplicon
attagaacattatgaaaacgatagtaaagacattaatgaacacataaactattggaaactggtgcgtatggtctgagcctgatggatttacagtgatcttcagtggtctgggg

SEQ ID NO: 2136 - DNA, 156 bp, Artificial Sequence
  amplicon
attagaacattatgaaaacgatagtaaagacattaatgaacacataaactattggaaactggtgcgtatggctccgggcagagcgcgtgtggcggccgagcacatgggcccgcgggccgggcgggctcggggcggccgggacgaggaggggcgacg

SEQ ID NO: 2137 - DNA, 101 bp, Artificial Sequence
  amplicon
caacccgcgacggcacatcccttgcagtaacactacgcctataactgctcgcggccgccaccgccgggccccggccgtccctggctcccctcctgcctcga

SEQ ID NO: 2138 - DNA, 108 bp, Artificial Sequence
  amplicon
caacccgcgacggcacatcccttgcagtaacactacgcctataacagcctcccgcgacgatgcccctcaacgttagcttcaccaacaggaactatgacctcgactacg

SEQ ID NO: 2139 - DNA, 108 bp, Artificial Sequence
  amplicon
caacccgcgacggcacatcccttgcagtaacactacgcctataaaggaggaacaagaagatgaggaagaaatcgatgttgtttctgtggaaaagaggcaggctcctgg

SEQ ID NO: 2140 - DNA, 90 bp, Artificial Sequence
  amplicon
caacccgcgacggcacatcccttgcagtaacactacgcctataactgaccatactccctggagccttctcccgaggtgcgcgggtgacct

SEQ ID NO: 2141 - DNA, 86 bp, Artificial Sequence
  amplicon
caacccgcgacggcacatcccttgcagtaacactacgcctataatctgagcctgatggatttacagtgatcttcagtggtctgggg

SEQ ID NO: 2142 - DNA, 129 bp, Artificial Sequence
  amplicon
caacccgcgacggcacatcccttgcagtaacactacgcctataactccgggcagagcgcgtgtggcggccgagcacatgggcccgcgggccgggcgggctcggggcggccgggacgaggaggggcgacg

SEQ ID NO: 2143 - DNA, 94 bp, Artificial Sequence
  amplicon
gcagcaaaccagtaacctgcaatggccgattcggaagctgctcgcggccgccaccgccgggccccggccgtccctggctcccctcctgcctcga

SEQ ID NO: 2144 - DNA, 101 bp, Artificial Sequence
  amplicon
gcagcaaaccagtaacctgcaatggccgattcggaagcagcctcccgcgacgatgcccctcaacgttagcttcaccaacaggaactatgacctcgactacg

SEQ ID NO: 2145 - DNA, 101 bp, Artificial Sequence
  amplicon
gcagcaaaccagtaacctgcaatggccgattcggaagaggaggaacaagaagatgaggaagaaatcgatgttgtttctgtggaaaagaggcaggctcctgg

SEQ ID NO: 2146 - DNA, 83 bp, Artificial Sequence
  amplicon
gcagcaaaccagtaacctgcaatggccgattcggaagctgaccatactccctggagccttctcccgaggtgcgcgggtgacct

SEQ ID NO: 2147 - DNA, 79 bp, Artificial Sequence
  amplicon
gcagcaaaccagtaacctgcaatggccgattcggaagtctgagcctgatggatttacagtgatcttcagtggtctgggg

SEQ ID NO: 2148 - DNA, 122 bp, Artificial Sequence
  amplicon
gcagcaaaccagtaacctgcaatggccgattcggaagctccgggcagagcgcgtgtggcggccgagcacatgggcccgcgggccgggcgggctcggggcggccgggacgaggaggggcgacg

SEQ ID NO: 2149 - DNA, 118 bp, Artificial Sequence
  amplicon
tatgaaaaagatagtaaatgcattatagatcacatagactattggaaagctgtacgacatgctgctcgcggccgccaccgccgggccccggccgtccctggctcccctcctgcctcga

SEQ ID NO: 2150 - DNA, 125 bp, Artificial Sequence
  amplicon
tatgaaaaagatagtaaatgcattatagatcacatagactattggaaagctgtacgacatgcagcctcccgcgacgatgcccctcaacgttagcttcaccaacaggaactatgacctcgactacg

SEQ ID NO: 2151 - DNA, 125 bp, Artificial Sequence
  amplicon
tatgaaaaagatagtaaatgcattatagatcacatagactattggaaagctgtacgacatgaggaggaacaagaagatgaggaagaaatcgatgttgtttctgtggaaaagaggcaggctcctgg

SEQ ID NO: 2152 - DNA, 107 bp, Artificial Sequence
  amplicon
tatgaaaaagatagtaaatgcattatagatcacatagactattggaaagctgtacgacatgctgaccatactccctggagccttctcccgaggtgcgcgggtgacct

SEQ ID NO: 2153 - DNA, 103 bp, Artificial Sequence
  amplicon
tatgaaaaagatagtaaatgcattatagatcacatagactattggaaagctgtacgacatgtctgagcctgatggatttacagtgatcttcagtggtctgggg

SEQ ID NO: 2154 - DNA, 146 bp, Artificial Sequence
  amplicon
tatgaaaaagatagtaaatgcattatagatcacatagactattggaaagctgtacgacatgctccgggcagagcgcgtgtggcggccgagcacatgggcccgcgggccgggcgggctcggggcggccgggacgaggaggggcgacg

SEQ ID NO: 2155 - DNA, 79 bp, Artificial Sequence
  amplicon
ggtgataaaactacgcctgtaactgctcgcggccgccaccgccgggccccggccgtccctggctcccctcctgcctcga

SEQ ID NO: 2156 - DNA, 86 bp, Artificial Sequence
  amplicon
ggtgataaaactacgcctgtaacagcctcccgcgacgatgcccctcaacgttagcttcaccaacaggaactatgacctcgactacg

SEQ ID NO: 2157 - DNA, 86 bp, Artificial Sequence
  amplicon
ggtgataaaactacgcctgtaaaggaggaacaagaagatgaggaagaaatcgatgttgtttctgtggaaaagaggcaggctcctgg

SEQ ID NO: 2158 - DNA, 68 bp, Artificial Sequence
  amplicon
ggtgataaaactacgcctgtaactgaccatactccctggagccttctcccgaggtgcgcgggtgacct

SEQ ID NO: 2159 - DNA, 64 bp, Artificial Sequence
  amplicon
ggtgataaaactacgcctgtaatctgagcctgatggatttacagtgatcttcagtggtctgggg

SEQ ID NO: 2160 - DNA, 107 bp, Artificial Sequence
  amplicon
ggtgataaaactacgcctgtaactccgggcagagcgcgtgtggcggccgagcacatgggcccgcgggccgggcgggctcggggcggccgggacgaggaggggcgacg

SEQ ID NO: 2161 - DNA, 95 bp, Artificial Sequence
  amplicon
gcgcatcatctaaataactgcaatggcatcacctgaagctgctcgcggccgccaccgccgggccccggccgtccctggctcccctcctgcctcga

SEQ ID NO: 2162 - DNA, 102 bp, Artificial Sequence
  amplicon
gcgcatcatctaaataactgcaatggcatcacctgaagcagcctcccgcgacgatgcccctcaacgttagcttcaccaacaggaactatgacctcgactacg

SEQ ID NO: 2163 - DNA, 102 bp, Artificial Sequence
  amplicon
gcgcatcatctaaataactgcaatggcatcacctgaagaggaggaacaagaagatgaggaagaaatcgatgttgtttctgtggaaaagaggcaggctcctgg

SEQ ID NO: 2164 - DNA, 84 bp, Artificial Sequence
  amplicon
gcgcatcatctaaataactgcaatggcatcacctgaagctgaccatactccctggagccttctcccgaggtgcgcgggtgacct

SEQ ID NO: 2165 - DNA, 80 bp, Artificial Sequence
  amplicon
gcgcatcatctaaataactgcaatggcatcacctgaagtctgagcctgatggatttacagtgatcttcagtggtctgggg

SEQ ID NO: 2166 - DNA, 123 bp, Artificial Sequence
  amplicon
gcgcatcatctaaataactgcaatggcatcacctgaagctccgggcagagcgcgtgtggcggccgagcacatgggcccgcgggccgggcgggctcggggcggccgggacgaggaggggcgacg

SEQ ID NO: 2167 - DNA, 85 bp, Artificial Sequence
  amplicon
attaactattggaattgtgtgcgactggctgctcgcggccgccaccgccgggccccggccgtccctggctcccctcctgcctcga

SEQ ID NO: 2168 - DNA, 92 bp, Artificial Sequence
  amplicon
attaactattggaattgtgtgcgactggcagcctcccgcgacgatgcccctcaacgttagcttcaccaacaggaactatgacctcgactacg

SEQ ID NO: 2169 - DNA, 92 bp, Artificial Sequence
  amplicon
attaactattggaattgtgtgcgactggaggaggaacaagaagatgaggaagaaatcgatgttgtttctgtggaaaagaggcaggctcctgg

SEQ ID NO: 2170 - DNA, 74 bp, Artificial Sequence
  amplicon
attaactattggaattgtgtgcgactggctgaccatactccctggagccttctcccgaggtgcgcgggtgacct

SEQ ID NO: 2171 - DNA, 70 bp, Artificial Sequence
  amplicon
attaactattggaattgtgtgcgactggtctgagcctgatggatttacagtgatcttcagtggtctgggg

SEQ ID NO: 2172 - DNA, 113 bp, Artificial Sequence
  amplicon
attaactattggaattgtgtgcgactggctccgggcagagcgcgtgtggcggccgagcacatgggcccgcgggccgggcgggctcggggcggccgggacgaggaggggcgacg

SEQ ID NO: 2173 - DNA, 95 bp, Artificial Sequence
  amplicon
aagacggagcctttgttgtggtgacactacacctatagctgctcgcggccgccaccgccgggccccggccgtccctggctcccctcctgcctcga

SEQ ID NO: 2174 - DNA, 102 bp, Artificial Sequence
  amplicon
aagacggagcctttgttgtggtgacactacacctatagcagcctcccgcgacgatgcccctcaacgttagcttcaccaacaggaactatgacctcgactacg

SEQ ID NO: 2175 - DNA, 102 bp, Artificial Sequence
  amplicon
aagacggagcctttgttgtggtgacactacacctatagaggaggaacaagaagatgaggaagaaatcgatgttgtttctgtggaaaagaggcaggctcctgg

SEQ ID NO: 2176 - DNA, 84 bp, Artificial Sequence
  amplicon
aagacggagcctttgttgtggtgacactacacctatagctgaccatactccctggagccttctcccgaggtgcgcgggtgacct

SEQ ID NO: 2177 - DNA, 80 bp, Artificial Sequence
  amplicon
aagacggagcctttgttgtggtgacactacacctatagtctgagcctgatggatttacagtgatcttcagtggtctgggg

SEQ ID NO: 2178 - DNA, 123 bp, Artificial Sequence
  amplicon
aagacggagcctttgttgtggtgacactacacctatagctccgggcagagcgcgtgtggcggccgagcacatgggcccgcgggccgggcgggctcggggcggccgggacgaggaggggcgacg

SEQ ID NO: 2179 - DNA, 88 bp, Artificial Sequence
  amplicon
acccagtaatctgcaatggccaattgtgaagctgctcgcggccgccaccgccgggccccggccgtccctggctcccctcctgcctcga

SEQ ID NO: 2180 - DNA, 95 bp, Artificial Sequence
  amplicon
acccagtaatctgcaatggccaattgtgaagcagcctcccgcgacgatgcccctcaacgttagcttcaccaacaggaactatgacctcgactacg

SEQ ID NO: 2181 - DNA, 95 bp, Artificial Sequence
  amplicon
acccagtaatctgcaatggccaattgtgaagaggaggaacaagaagatgaggaagaaatcgatgttgtttctgtggaaaagaggcaggctcctgg

SEQ ID NO: 2182 - DNA, 77 bp, Artificial Sequence
  amplicon
acccagtaatctgcaatggccaattgtgaagctgaccatactccctggagccttctcccgaggtgcgcgggtgacct

SEQ ID NO: 2183 - DNA, 73 bp, Artificial Sequence
  amplicon
acccagtaatctgcaatggccaattgtgaagtctgagcctgatggatttacagtgatcttcagtggtctgggg

SEQ ID NO: 2184 - DNA, 116 bp, Artificial Sequence
  amplicon
acccagtaatctgcaatggccaattgtgaagctccgggcagagcgcgtgtggcggccgagcacatgggcccgcgggccgggcgggctcggggcggccgggacgaggaggggcgacg

SEQ ID NO: 2185 - DNA, 93 bp, Artificial Sequence
  amplicon
gtgatcatattgatcattggaaacacgtgcgacatgctgctcgcggccgccaccgccgggccccggccgtccctggctcccctcctgcctcga

SEQ ID NO: 2186 - DNA, 100 bp, Artificial Sequence
  amplicon
gtgatcatattgatcattggaaacacgtgcgacatgcagcctcccgcgacgatgcccctcaacgttagcttcaccaacaggaactatgacctcgactacg

SEQ ID NO: 2187 - DNA, 100 bp, Artificial Sequence
  amplicon
gtgatcatattgatcattggaaacacgtgcgacatgaggaggaacaagaagatgaggaagaaatcgatgttgtttctgtggaaaagaggcaggctcctgg

SEQ ID NO: 2188 - DNA, 82 bp, Artificial Sequence
  amplicon
gtgatcatattgatcattggaaacacgtgcgacatgctgaccatactccctggagccttctcccgaggtgcgcgggtgacct

SEQ ID NO: 2189 - DNA, 78 bp, Artificial Sequence
  amplicon
gtgatcatattgatcattggaaacacgtgcgacatgtctgagcctgatggatttacagtgatcttcagtggtctgggg

SEQ ID NO: 2190 - DNA, 121 bp, Artificial Sequence
  amplicon
gtgatcatattgatcattggaaacacgtgcgacatgctccgggcagagcgcgtgtggcggccgagcacatgggcccgcgggccgggcgggctcggggcggccgggacgaggaggggcgacg

SEQ ID NO: 2191 - DNA, 96 bp, Artificial Sequence
  amplicon
cctgtacccagtgtactacacataatgttgcgccaatagctgctcgcggccgccaccgccgggccccggccgtccctggctcccctcctgcctcga

SEQ ID NO: 2192 - DNA, 103 bp, Artificial Sequence
  amplicon
cctgtacccagtgtactacacataatgttgcgccaatagcagcctcccgcgacgatgcccctcaacgttagcttcaccaacaggaactatgacctcgactacg

SEQ ID NO: 2193 - DNA, 103 bp, Artificial Sequence
  amplicon
cctgtacccagtgtactacacataatgttgcgccaatagaggaggaacaagaagatgaggaagaaatcgatgttgtttctgtggaaaagaggcaggctcctgg

SEQ ID NO: 2194 - DNA, 85 bp, Artificial Sequence
  amplicon
cctgtacccagtgtactacacataatgttgcgccaatagctgaccatactccctggagccttctcccgaggtgcgcgggtgacct

SEQ ID NO: 2195 - DNA, 81 bp, Artificial Sequence
  amplicon
cctgtacccagtgtactacacataatgttgcgccaatagtctgagcctgatggatttacagtgatcttcagtggtctgggg

SEQ ID NO: 2196 - DNA, 124 bp, Artificial Sequence
  amplicon
cctgtacccagtgtactacacataatgttgcgccaatagctccgggcagagcgcgtgtggcggccgagcacatgggcccgcgggccgggcgggctcggggcggccgggacgaggaggggcgacg

SEQ ID NO: 2197 - DNA, 102 bp, Artificial Sequence
  amplicon
gccccaactgttccagaaacctataaaagaagatggctgattcagctgctcgcggccgccaccgccgggccccggccgtccctggctcccctcctgcctcga

SEQ ID NO: 2198 - DNA, 109 bp, Artificial Sequence
  amplicon
gccccaactgttccagaaacctataaaagaagatggctgattcagcagcctcccgcgacgatgcccctcaacgttagcttcaccaacaggaactatgacctcgactacg

SEQ ID NO: 2199 - DNA, 109 bp, Artificial Sequence
  amplicon
gccccaactgttccagaaacctataaaagaagatggctgattcagaggaggaacaagaagatgaggaagaaatcgatgttgtttctgtggaaaagaggcaggctcctgg

SEQ ID NO: 2200 - DNA, 91 bp, Artificial Sequence
  amplicon
gccccaactgttccagaaacctataaaagaagatggctgattcagctgaccatactccctggagccttctcccgaggtgcgcgggtgacct

SEQ ID NO: 2201 - DNA, 87 bp, Artificial Sequence
  amplicon
gccccaactgttccagaaacctataaaagaagatggctgattcagtctgagcctgatggatttacagtgatcttcagtggtctgggg

SEQ ID NO: 2202 - DNA, 130 bp, Artificial Sequence
  amplicon
gccccaactgttccagaaacctataaaagaagatggctgattcagctccgggcagagcgcgtgtggcggccgagcacatgggcccgcgggccgggcgggctcggggcggccgggacgaggaggggcgacg

SEQ ID NO: 2203 - DNA, 115 bp, Artificial Sequence
  amplicon
gaactggacagtgataaattagtagatcaaattaattattggacgttggtacgatatgctgctcgcggccgccaccgccgggccccggccgtccctggctcccctcctgcctcga

SEQ ID NO: 2204 - DNA, 122 bp, Artificial Sequence
  amplicon
gaactggacagtgataaattagtagatcaaattaattattggacgttggtacgatatgcagcctcccgcgacgatgcccctcaacgttagcttcaccaacaggaactatgacctcgactacg

SEQ ID NO: 2205 - DNA, 122 bp, Artificial Sequence
  amplicon
gaactggacagtgataaattagtagatcaaattaattattggacgttggtacgatatgaggaggaacaagaagatgaggaagaaatcgatgttgtttctgtggaaaagaggcaggctcctgg

SEQ ID NO: 2206 - DNA, 104 bp, Artificial Sequence
  amplicon
gaactggacagtgataaattagtagatcaaattaattattggacgttggtacgatatgctgaccatactccctggagccttctcccgaggtgcgcgggtgacct

SEQ ID NO: 2207 - DNA, 100 bp, Artificial Sequence
  amplicon
gaactggacagtgataaattagtagatcaaattaattattggacgttggtacgatatgtctgagcctgatggatttacagtgatcttcagtggtctgggg

SEQ ID NO: 2208 - DNA, 143 bp, Artificial Sequence
  amplicon
gaactggacagtgataaattagtagatcaaattaattattggacgttggtacgatatgctccgggcagagcgcgtgtggcggccgagcacatgggcccgcgggccgggcgggctcggggcggccgggacgaggaggggcgacg

SEQ ID NO: 2209 - DNA, 115 bp, Artificial Sequence
  amplicon
ggaactgcaggcccaaacaccggagggcacctcagtgcaactaaaactgcgtttatagctgctcgcggccgccaccgccgggccccggccgtccctggctcccctcctgcctcga

SEQ ID NO: 2210 - DNA, 122 bp, Artificial Sequence
  amplicon
ggaactgcaggcccaaacaccggagggcacctcagtgcaactaaaactgcgtttatagcagcctcccgcgacgatgcccctcaacgttagcttcaccaacaggaactatgacctcgactacg

SEQ ID NO: 2211 - DNA, 122 bp, Artificial Sequence
  amplicon
ggaactgcaggcccaaacaccggagggcacctcagtgcaactaaaactgcgtttatagaggaggaacaagaagatgaggaagaaatcgatgttgtttctgtggaaaagaggcaggctcctgg

SEQ ID NO: 2212 - DNA, 104 bp, Artificial Sequence
  amplicon
ggaactgcaggcccaaacaccggagggcacctcagtgcaactaaaactgcgtttatagctgaccatactccctggagccttctcccgaggtgcgcgggtgacct

SEQ ID NO: 2213 - DNA, 100 bp, Artificial Sequence
  amplicon
ggaactgcaggcccaaacaccggagggcacctcagtgcaactaaaactgcgtttatagtctgagcctgatggatttacagtgatcttcagtggtctgggg

SEQ ID NO: 2214 - DNA, 143 bp, Artificial Sequence
  amplicon
ggaactgcaggcccaaacaccggagggcacctcagtgcaactaaaactgcgtttatagctccgggcagagcgcgtgtggcggccgagcacatgggcccgcgggccgggcgggctcggggcggccgggacgaggaggggcgacg

SEQ ID NO: 2215 - DNA, 78 bp, Artificial Sequence
  amplicon
catcggcaatggacagtgaagctgctcgcggccgccaccgccgggccccggccgtccctggctcccctcctgcctcga

SEQ ID NO: 2216 - DNA, 85 bp, Artificial Sequence
  amplicon
catcggcaatggacagtgaagcagcctcccgcgacgatgcccctcaacgttagcttcaccaacaggaactatgacctcgactacg

SEQ ID NO: 2217 - DNA, 85 bp, Artificial Sequence
  amplicon
catcggcaatggacagtgaagaggaggaacaagaagatgaggaagaaatcgatgttgtttctgtggaaaagaggcaggctcctgg

SEQ ID NO: 2218 - DNA, 67 bp, Artificial Sequence
  amplicon
catcggcaatggacagtgaagctgaccatactccctggagccttctcccgaggtgcgcgggtgacct

SEQ ID NO: 2219 - DNA, 63 bp, Artificial Sequence
  amplicon
catcggcaatggacagtgaagtctgagcctgatggatttacagtgatcttcagtggtctgggg

SEQ ID NO: 2220 - DNA, 106 bp, Artificial Sequence
  amplicon
catcggcaatggacagtgaagctccgggcagagcgcgtgtggcggccgagcacatgggcccgcgggccgggcgggctcggggcggccgggacgaggaggggcgacg

SEQ ID NO: 2221 - DNA, 106 bp, Artificial Sequence
  amplicon
gagcggttccgctgccctgaggcactcttccagccttccttcctgggcatggagtcctgtggcatccacgaaactaccttcaactccatcatgaagtgtgacgtgg

SEQ ID NO: 2222 - DNA, 109 bp, Artificial Sequence
  amplicon
gtgaaggagggttggctgcacaaacgaggggagtacatcaagacctggcggccacgctacttcctcctcaagaatgatggcaccttcattggctacaaggagcggccgc

SEQ ID NO: 2223 - DNA, 106 bp, Artificial Sequence
  amplicon
ctctttctggcctggaggctatccagcgtactccaaagattcaggtttactcacgtcatccagcagagaatggaaagtcaaatttcctgaattgctatgtgtctgg

SEQ ID NO: 2224 - DNA, 104 bp, Artificial Sequence
  amplicon
ggcacctgcacacctggatccaggataacggaggctgggatgcctttgtggaactgtacggccccagcatgcggcctctgtttgatttctcctggctgtctctg

SEQ ID NO: 2225 - DNA, 109 bp, Artificial Sequence
  amplicon
gagattcctgatgggcagattacagtgggacaaagaattggatctggatcatttggaacagtctacaagggaaagtggcatggtgatgtggcagtgaaaatgttgaatg

SEQ ID NO: 2226 - DNA, 104 bp, Artificial Sequence
  amplicon
ccgaggactttggcgtgggccaggaaatcacatcctacactgcccaggagccagacacatttatggaacagaaaataacatatcggatttggagagacactgcc

SEQ ID NO: 2227 - DNA, 93 bp, Artificial Sequence
  amplicon
cggaggccgatccaggtcatgatgatgggcagcgcccgagtggcggagctgctgctgctccacggcgcggagcccaactgcgccgaccccgcc

SEQ ID NO: 2228 - DNA, 103 bp, Artificial Sequence
  amplicon
ccgtttcgggaggcgcgcgatccaggtcatgatgatgggcagcgcccgcgtggcggagctgctgctgctccacggcgcggagcccaactgcgcagaccctgcc

SEQ ID NO: 2229 - DNA, 105 bp, Artificial Sequence
  amplicon
ggggacgaattctgcacaatggcgcctactcgctgaccctgcaagggctgggcatcagctggctggggctgcgctcactgagggaactgggcagtggactggccc

SEQ ID NO: 2230 - DNA, 104 bp, Artificial Sequence
  amplicon
agatggctgcagccaaatgccgcaaccggaggagggagctgactgatacactccaagcggagacagaccaactagaagatgagaagtctgctttgcagaccgag

SEQ ID NO: 2231 - DNA, 105 bp, Artificial Sequence
  amplicon
caccatggagaaggctggggctcatttgcaggggggagccaaaagggtcatcatctctgccccctctgctgatgcccccatgttcgtcatgggtgtgaaccatga

SEQ ID NO: 2232 - DNA, 105 bp, Artificial Sequence
  amplicon
ttgcagggtttcaccaggatccacctctgatgttcactgaagagtaccagaaaagtctgctagagcagtaccatctgggtctggatcaaaaacgcagaaaatacg

SEQ ID NO: 2233 - DNA, 107 bp, Artificial Sequence
  amplicon
gggcgccggcggtgtgggcaagagtgcgctgaccatccagctgatccagaaccattttgtggacgaatacgaccccactatagaggattcctaccggaagcaggtgg

SEQ ID NO: 2234 - DNA, 101 bp, Artificial Sequence
  amplicon
tgaggactggggagggctttctttgtgtatttgccataaataatactaaatcatttgaagatattcaccattatagagaacaaattaaaagagttaaggac

SEQ ID NO: 2235 - DNA, 106 bp, Artificial Sequence
  amplicon
ggctgacctggagatgcaaattgagagcctgactgaagagctggcctatctgaagaagaaccacgaggaggaaatgaaagaccttcgaaatgtgtccactggtgat

SEQ ID NO: 2236 - DNA, 107 bp, Artificial Sequence
  amplicon
gaagaaccacgaggaggagatgaacgccctgcgaggccaggtgggtggtgagatcaatgtggagatggacgctgccccaggcgtggacctgagccgcatcctcaacg

SEQ ID NO: 2237 - DNA, 107 bp, Artificial Sequence
  amplicon
atccgtgactggtaccagaggcaggccccggggcccgcccgtgactacagccagtactacaggacaattgaggagctgcagaacaagatcctcacagccaccgtgga

SEQ ID NO: 2238 - DNA, 103 bp, Artificial Sequence
  amplicon
cgacttcttcaacaagatcgtcaacaaaaacaatgtgagatgtctccagcatttttacggacccaatcatgagcactgctttaataggacacttctgagaaat

SEQ ID NO: 2239 - DNA, 105 bp, Artificial Sequence
  amplicon
ctcttctgaccctgatgagaaagctcaagattccaaggcctattcaaaaatcactgaaggaaaagtttcaggaaatcctcaggtacatatcaagaatgtcaaaga

SEQ ID NO: 2240 - DNA, 109 bp, Artificial Sequence
  amplicon
gcagctgcttagacgctggatttttttcgggtagtggaaaaccagcagcctcccgcgacgatgcccctcaacgttagcttcaccaacaggaactatgacctcgactacg

SEQ ID NO: 2241 - DNA, 110 bp, Artificial Sequence
  amplicon
ccagatcctgatccggaaccgagccacagacctggatgcccgcatgcatgatggcacgacgccactgatcctggctgcccgcctggccgtggagggcatgctggaggacc

SEQ ID NO: 2242 - DNA, 102 bp, Artificial Sequence
  amplicon
ggagaacttggaaatggaaacattaaattgtcacagacaagtaatgtcgataaagaggaggaagctgttaccatagagatgaatgaaccagttcaactaact

SEQ ID NO: 2243 - DNA, 105 bp, Artificial Sequence
  amplicon
tatctagtacttactttaacaaaaaatgatcttgacaaagcaaataaagacaaagccaaccgatacttttctccaaattttaaggtgaagctgtacttcacaaaa

SEQ ID NO: 2244 - DNA, 101 bp, Artificial Sequence
  amplicon
aaacaaatattttgcagtatgcttccaccaggccccctaccttgtcaccaatacctcacattcctcgaagcccttacaagtttcctagttcacccttacgg

SEQ ID NO: 2245 - DNA, 104 bp, Artificial Sequence
  amplicon
gctgaaaaggtcaaggccttcttggctgatccatctgcctttgtggctgctgcccctgtggctgctgccaccacagctgctcctgctgctgctgcagccccagc

SEQ ID NO: 2246 - DNA, 109 bp, Artificial Sequence
  amplicon
agtgctgagttggcagttttcttctgtcaccaaaagaggtctcaatgtggaccagctgaacatgttgggagagaagcttcttggtcctaacgccagccccgatggtctc

SEQ ID NO: 2247 - DNA, 106 bp, Artificial Sequence
  amplicon
ttcttgttggtgacacctcacctcacccacgcgaaaaccttcctcaggaccctggtccgaggtgtccctgagtatggctgcgtggtgaacttgcggaagacagtgg

SEQ ID NO: 2248 - DNA, 103 bp, Artificial Sequence
  amplicon
ccaactttgatgtgcgtgaaattgtaaataacatcaggcgtttgatggatggagaagaacctttgccaatgcttccaagttacaagaacttcaagggtactat

SEQ ID NO: 2249 - DNA, 107 bp, Artificial Sequence
  amplicon
tcccttcccagaaaacctaccagggcagctacggtttccgtctgggcttcttgcattctgggacagccaagtctgtgacttgcacgtactcccctgccctcaacaag

SEQ ID NO: 2250 - DNA, 108 bp, Artificial Sequence
  amplicon
ccagggccccacctcttcggcaagatcgtcaaccgaggctgtcgagaaacggcgtttatcttcgctatcacctccgccggggtcacccattcggtggcgcgctcctgc

SEQ ID NO: 2251 - DNA, 18 bp, Artificial Sequence
  primer
tgcagatgctgtgtctgg

SEQ ID NO: 2252 - DNA, 17 bp, Artificial Sequence
  primer
cgtactggcccaggacc

SEQ ID NO: 2253 - DNA, 20 bp, Artificial Sequence
  primer
gaagatggtgatgggatttc

SEQ ID NO: 2254 - DNA, 20 bp, Artificial Sequence
  primer
atgcaccaaaagagaactgc

SEQ ID NO: 2255 - DNA, 20 bp, Artificial Sequence
  primer
ttacagctgggtttctctac

SEQ ID NO: 2256 - DNA, 25 bp, Artificial Sequence
  primer
gtaaccttttgttgcaagtgtgact

SEQ ID NO: 2257 - DNA, 25 bp, Artificial Sequence
  primer
gattatggtttctgagaacagatgg

SEQ ID NO: 2258 - DNA, 20 bp, Artificial Sequence
  primer
catcgagcacggcatcgtca

SEQ ID NO: 2259 - DNA, 21 bp, Artificial Sequence
  primer
tagcacagcctggatagcaac

SEQ ID NO: 2260 - DNA, 19 bp, Artificial Sequence
  primer
gaaggtgaaggtcggagtc

SEQ ID NO: 2261 - DNA, 20 bp, Artificial Sequence
  primer
gaagatggtgatgggatttc

(19) United States
(12) Patent Application Publication
(10) Pub. No.: US 2022/0205057 A1
(43) Pub. Date: Jun. 30, 2022

(54) COMPOSITION OF PRIMERS FOR DETECTING HIGH GRADE SQUAMOUS INTRAEPITHELIAL LESION

(71) Applicants: INSTITUT PASTEUR, PARIS (FR); ECOLE NATIONALE VETERINAIRE D'ALFORT, Maisons-Alfort (FR)

(72) Inventors: Marc ELOIT, PARIS (FR); Philippe PEROT, PARIS (FR); Anne Emmanuelle Marie BITON, PARIS (FR)

(21) Appl. No.: 17/593,447

(22) PCT Filed: Mar. 16, 2020
(86) PCT No.: PCT/EP2020/057078

§ 371 Date: Sep. 19, 2021

(30) Foreign Application Priority Data
Mar. 28, 2019 (EP) 19305394.9

(51) Int. Cl. C12Q 1/70; C12Q 1/6883

(52) U.S. Cl. CPC C12Q 1/708; C12Q 1/6883; C12Q 2600/16; C12Q 2600/156; C12Q 2600/112

(57) ABSTRACT

The present invention relates to a composition of primers for detecting HSIL comprising a first set of primers, called splice junctions set of primers which comprises at least 2 pairs of primers of each of a first subset of pairs of primers specific of HPV16, a second subset specific of HPV18, a third subset specific of HPV31, a fourth subset specific of HPV33, a fifth subset specific of HPV35, a sixth subset specific of HPV39, a seventh subset specific of HPV45, a eighth subset specific of HPV51, a ninth subset specific of HPV52, a tenth subset specific of HPV56, an eleventh subset specific of HPV58, a twelfth subset specific of HPV59 and a thirteenth subset specific of HPV66.

### FIELD OF THE INVENTION

The present invention relates to a composition of primers, a kit and a method for detecting high grade squamous intraepithelial lesion (HSIL) and/or for typing a Human Papillomavirus (HPV).

### BACKGROUND OF THE INVENTION

Human papillomaviruses (HPV) infections are associated with the development of cervical carcinoma, one of the most common cancers among women, and other cancers like anal cancer (Lin C et al. Human papillomavirus types from infection to cancer in the anus, according to sex and HIV status: a systematic review and meta-analysis. Lancet Infect Dis, 2018, 18:198-206) and head and neck cancer (Chaturvedi A K, et al. Human papillomavirus and rising oropharyngeal cancer incidence in the United States. J Clin Oncol Off J Am Soc Clin Oncol, 2011, 29:4294-301). HPV are the etiologic agents responsible for over 99% of all cervical cancers (Walboomers J M, et al. Human papillomavirus is a necessary cause of invasive cervical cancer worldwide. J Pathol, 1999, 189:12-9). HPV are small, non-enveloped DNA viruses commonly transmitted through sexual contact, which infect basal cells and replicate in the nucleus of squamous epithelial cells. HPV include more than 200 genotypes characterized by their oncogenic potential, with highly oncogenic HPV types (high-risk HPV) having a unique ability to drive cell proliferation (Schiffman M, et al. S. Carcinogenic human papillomavirus infection. Nat Rev Dis Primer, 2016, 2:16086).

The genomic organization of papillomaviruses is divided into functional early and late regions. The model of HPV infection, which is mainly derived from knowledge on HPV16, is that following the infection of basal cells in the cervical epithelium, the early HPV genes (E6, E7, E1, E2, E4 and E5) are expressed and the viral DNA replicates from the episomal form of the viral DNA. As the cells divide, in the upper layers of the epithelium the viral genome is replicated further, and the late genes (L1 and L2) and E4 are expressed. Viral shedding then further initiates new infections (Woodman C B J, et al. The natural history of cervical HPV infection: unresolved issues. Nat Rev Cancer, 2007, 7:11-22).

HPV infection during the development of cervical cancer is associated with a shift from productive infection (which in most of the cases will be cleared by the immune system), towards non-productive persistent and transforming infection (in a minority of cases) characterized in particular by a high level of E6 and E7 mRNAs and low expression of E2 and late genes such as L1 (Doorbar J, et al. The biology and life-cycle of human papillomaviruses. Vaccine, 2012, 30 Suppl 5:F55-70, Shulzhenko N, et al. Ménage à trois: an evolutionary interplay between human papillomavirus, a tumor, and a woman. Trends Microbiol, 2014, 22:345-53). High-risk HPV infection may result in low-grade lesions, with highly productive infection and high rate of spontaneous regression. In contrast, high-risk persistent HPV infection is responsible for high-grade lesion, the true precancerous lesion.

Cervical cancer screening allows detection and treatment of precancerous lesions before the development of cervical cancer. Screening is based on different algorithms, some allowing detection of HPV, and others identifying abnormal cells. Despite the role of high-risk HPV in cervical cancer, screening tests of cancer or precancerous lesions remain in many countries mainly based on the Papanicolaou (Pap) cytology test and do not include molecular virology tests (Schiffman M, et al. 2016). This is largely due to the low Positive Predictive Value (PPV) of current molecular tests. Indeed, because most of the current molecular diagnostic methods rely on the detection of HPV genome (DNA) and do not address the patterns of viral expression (RNA), they remain weak predictors of the evolution from low-grade squamous intraepithelial lesion (LSIL) to high-grade squamous intraepithelial lesion (HSIL) of the cervix (Tornesello M L, et al. Viral and cellular biomarkers in the diagnosis of cervical intraepithelial neoplasia and cancer. BioMed Res Int, 2013, 2013:519619). In addition, DNA identification of high-risk HPV is not fully predictive of cancer since only persistence for years of high-risk HPV is associated with an increased risk of cancer development (Schiffman M, et al. 2016). Thus, the use of HPV DNA tests, as a screening assay, is currently increasing worldwide and shows high sensitivity (Ogilvie G S, et al. Effect of Screening With Primary Cervical HPV Testing vs Cytology Testing on High-grade Cervical Intraepithelial Neoplasia at 48 Months: The HPV FOCAL Randomized Clinical Trial. JAMA, 2018, 320:43-52) but low PPV for HSIL detection (Cuzick J, et al. Comparing the performance of six human papillomavirus tests in a screening population. Br J Cancer, 2013, 108:908-13).

HPV RNA tests and in particular expression of E6 and E7 mRNAs of high-risk HPV have been proposed as better molecular markers of cancer development, but E6 and E7 are also expressed during HPV transient infection so it remains difficult to define a threshold of expression associated with the persistence and evolution to high-grade lesions and cancer. There is no consensus that HPV RNA tests have a better diagnostic accuracy compared to HPV DNA tests and cytology for the detection of cervical precancerous lesions (Virtanen E, et al. Performance of mRNA- and DNA-based high-risk human papillomavirus assays in detection of high-grade cervical lesions. Acta Obstet Gynecol Scand, 2017, 96:61-8, Cook D A, et al. Aptima HPV Assay versus Hybrid Capture® 2 HPV test for primary cervical cancer screening in the HPV FOCAL trial. J Clin Virol Off Publ Pan Am Soc Clin Virol, 2017, 87:23-9, Ge Y et al. Aptima Human Papillomavirus E6/E7 mRNA Test Results Strongly Associated With Risk for High-Grade Cervical Lesions in Follow-Up Biopsies. J Low Genit Tract Dis, 2018, 22:195-200). There is therefore a need for a novel generation of molecular diagnostic tests that can not only detect HPV infection, but also have the ability to accurately predict precancerous stages to offer a better and cost saving medical benefit (de Thurah L, et al. Concordant testing results between various human papillomavirus assays in primary cervical cancer screening: systematic review. Clin Microbiol Infect Off Publ Eur Soc Clin Microbiol Infect Dis, 2018, 24:29-36, Hawkes D, et al. Not all HPV nucleic acid tests are equal: only those calibrated to detect high grade lesions matter for cervical screening. Clin Microbiol Infect Off Publ Eur Soc Clin Microbiol Infect Dis, 2018, 24:436-7, de Thurah L, et al. Not all HPV nucleic acid tests are equal: only those calibrated to detect high grade lesions matter for cervical screening: Response to “Concordant testing results between various human papillomavirus assays in primary cervical cancer screening: systematic review” Published 27 May, 2017. Clin Microbiol Infect Off Publ Eur Soc Clin Microbiol Infect Dis, 2018, 24:438-9).

### SUMMARY OF THE INVENTION

Now, taking advantage of Next-Generation Sequencing (NGS) technologies, the inventors have developed a multiplexed amplification system targeting the virus splice junctions coupled with NGS analysis that allows to describe fine equilibrium among transcript species of 13 high-risk HPV (HPV16, 18, 31, 33, 35, 39, 45, 51, 52, 56, 58, 59, 66) plus 3 putative high-risk HPV (HPV68, 73, 82), in a single reaction. This molecular approach makes, in particular, possible to take a snapshot of the early vs late populations of HPV transcripts and to define a model based on a combination of reads that reflects the biology of the virus, which can then be correlated to the evolution of lesions. The ultimate goal is to replace the conventional methods of the triage of women at risk of transforming infection before colposcopy.

Based on a study conducted on 55 patients, starting from cervical smears conserved at room temperature, the inventors have showed that the method of the invention can be used as a marker of high-grade cytology, with encouraging diagnostic performances as a triage test.

A subject of the present invention is therefore a composition of primers for detecting high grade squamous intraepithelial lesion (HSIL) comprising a first set of primers, called splice junctions set of primers, which comprises:
at least 2 pairs of primers of a first subset of HPV16 specific pairs of primers having the nucleic acid sequence selected from the group consisting of SEQ ID NO: 1-2 to SEQ ID NO: 27-28; and
at least 2 pairs of primers of a second subset of HPV18 specific pairs of primers having the nucleic acid sequence selected from the group consisting of SEQ ID NO: 29-30 to SEQ ID NO: 63-64; and
at least 2 pairs of primers of a third subset of HPV31 specific pairs of primers having the nucleic acid sequence selected from the group consisting of SEQ ID NO: 65-66 to SEQ ID NO: 91-92; and
at least 2 pairs of primers of a fourth subset of HPV33 specific pairs of primers having the nucleic acid sequence selected from the group consisting of SEQ ID NO: 93-94 to SEQ ID NO: 117-118; and
at least 2 pairs of primers of a fifth subset of HPV35 specific pairs of primers having the nucleic acid sequence selected from the group consisting of SEQ ID NO: 119-120 to SEQ ID NO: 145-146; and
at least 2 pairs of primers of a sixth subset of HPV39 specific pairs of primers having the nucleic acid sequence selected from the group consisting of SEQ ID NO: 147-148 to SEQ ID NO: 165-166; and
at least 2 pairs of primers of a seventh subset of HPV45 specific pairs of primers having the nucleic acid sequence selected from the group consisting of SEQ ID NO: 167-168 to SEQ ID NO: 193-194; and
at least 2 pairs of primers of a eighth subset of HPV51 specific pairs of primers having the nucleic acid sequence selected from the group consisting of SEQ ID NO: 195-196 to SEQ ID NO: 213-214; and
at least 2 pairs of primers of a ninth subset of HPV52 specific pairs of primers having the nucleic acid sequence selected from the group consisting of SEQ ID NO: 215-216 to SEQ ID NO: 245-246; and
at least 2 pairs of primers of a tenth subset of HPV56 specific pairs of primers having the nucleic acid sequence selected from the group consisting of SEQ ID NO: 247-248 to SEQ ID NO: 277-278; and
at least 2 pairs of primers of an eleventh subset of HPV58 specific pairs of primers having the nucleic acid sequence selected from the group consisting of SEQ ID NO: 279-280 to SEQ ID NO: 303-304; and
at least 2 pairs of primers of a twelfth subset of HPV59 specific pairs of primers having the nucleic acid sequence selected from the group consisting of SEQ ID NO: 305-306 to SEQ ID NO: 331-332; and
at least 2 pairs of primers of a thirteenth subset of HPV66 specific pairs of primers having the nucleic acid sequence selected from the group consisting of SEQ ID NO: 333-334 to SEQ ID NO: 361-362.

The aim of the invention is notably to lower the number of primers used in the multiplex system. This lowering of the numbers of primers may be done by lowering the number of the targeted splice junctions and by using redundant nucleic acid sequences.

Thus the 362 nucleic acid sequences of the primers of the splice junctions set primers are redundant and represent in fact 165 unique nucleic acid sequences.

The present invention also relates to a composition of primers for detecting HSIL comprising a first set of pairs of primers, called splice junctions set of primers, which comprises:
at least 2 pairs of primers of a first subset of HPV16 specific pairs of primers able to produce the amplicons having the nucleic acid sequence selected from the group consisting of SEQ ID NO: 1501 to SEQ ID NO: 1514; and
at least 2 pairs of primers of a second subset of HPV18 specific pairs of primers able to produce the amplicons having the nucleic acid sequence selected from the group consisting of SEQ ID NO: 1515 to SEQ ID NO: 1532; and
at least 2 pairs of primers of a third subset of HPV31 specific pairs of primers able to produce the amplicons having the nucleic acid sequence selected from the group consisting of SEQ ID NO: 1533 to SEQ ID NO: 1546; and
at least 2 pairs of primers of a fourth subset of HPV33 specific pairs of primers able to produce the amplicons having the nucleic acid sequence selected from the group consisting of SEQ ID NO:1547 to SEQ ID NO:1559; and
at least 2 pairs of primers of a fifth subset of HPV35 specific pairs of primers able to produce the amplicons having the nucleic acid sequence selected from the group consisting of SEQ ID NO: 1560 to SEQ ID NO: 1573; and
at least 2 pairs of primers of a sixth subset of HPV39 specific pairs of primers able to produce the amplicons having the nucleic acid sequence selected from the group consisting of SEQ ID NO: 1574 to SEQ ID NO: 1583; and
at least 2 pairs of primers of a seventh subset of HPV45 specific pairs of primers able to produce the amplicons having the nucleic acid sequence selected from the group consisting of SEQ ID NO: 1584 to SEQ ID NO: 1597; and
at least 2 pairs of primers of a eighth subset of HPV51 specific pairs of primers able to produce the amplicons having the nucleic acid sequence selected from the group consisting of SEQ ID NO: 1598 to SEQ ID NO: 1607; and
at least 2 pairs of primers of a ninth subset of HPV52 specific pairs of primers able to produce the amplicons having the nucleic acid sequence selected from the group consisting of SEQ ID NO: 1608 to SEQ ID NO: 1623; and
at least 2 pairs of primers of a tenth subset of HPV56 specific pairs of primers able to produce the amplicons having the nucleic acid sequence selected from the group consisting of SEQ ID NO: 1624 to SEQ ID NO: 1639; and
at least 2 pairs of primers of an eleventh subset of HPV58 specific pairs of primers able to produce the amplicons having the nucleic acid sequence selected from the group consisting of SEQ ID NO:1640 to SEQ ID NO: 1652; and
at least 2 pairs of primers of a twelfth subset of HPV59 specific pairs of primers able to produce the amplicons having the nucleic acid sequence selected from the group consisting of SEQ ID NO: 1653 to SEQ ID NO: 1666; and
at least 2 pairs of primers of a thirteenth subset of HPV66 specific pairs of primers able to produce the amplicons having the nucleic acid sequence selected from the group consisting of SEQ ID NO: 1667 to SEQ ID NO: 1681.

### DETAILED DESCRIPTION OF THE INVENTION

Definitions

High-risk HPV also called HR-HPV herein refer to the HPV of the following types: HPV16, HPV18, HPV31, HPV33, HPV35, HPV39, HPV45, HPV51, HPV52, HPV56, HPV58, HPV59 and HPV66.

Putative high risk HPV herein refer to the HPV of the following types: HPV68, HPV73 and HPV82.

HSIL refers to high grade squamous intraepithelial lesion. HSIL may be cervical, anogenital, head and neck HSIL. Preferably, HSIL is cervix HSIL.

LSIL refers to low grade squamous intraepithelial lesion. LSIL may be cervical, anogenital, head and neck LSIL. Preferably, LSIL is cervix LSIL.

Splice junctions set of primers refer herein to a set of primers which target high risk and optionally putative high risk HPV splice events involving a pair of splice donor (SD) and splice acceptor (SA) sites.

Unsplice junctions set of primers refer herein to a set of primers which target high risk and optionally putative high risk HPV genomic regions spanning either splice donor or splice acceptor sites in the absence of any splice event. In this context, the term “junction” refers to exon-intron interface (i.e. the position where a donor or acceptor site would be found in case of a splice event).

Genomic set of primers refer herein to a set of primers which target high risk and optionally of putative high risk HPV genomic regions away from any splice donor or splice acceptor sites.

Fusion set of primers refer herein to a set of primers which target high risk and optionally of putative high risk HPV fusion transcripts.

Human set of primers refer herein to a set of primers which target human sequences.

HPV RNA Seq refers herein to a multiplexed amplification system coupled with Next Generation Sequencing analysis.

The expression “the nucleic acid sequence selected from the group consisting of SEQ ID NO: x-x+1 to SEQ ID NO: x+n-x+n+1” means “the nucleic acid sequence selected from the group consisting of SEQ ID NO: x-x+1, SEQ ID NO: x+2-x+3, SEQ ID NO: x+4-x+5 . . . and SEQ ID NO: x+n-x+n+1”. For example, the nucleic acid sequence selected from the group consisting of SEQ ID NO: 1-2 to SEQ ID NO: 5-6 means the nucleic acid sequence selected from the group consisting of SEQ ID NO: 1-2 (pair of primers SEQ ID NO: 1 and SEQ ID NO: 2), SEQ ID NO: 3-4 (pair of primers SEQ ID NO: 3 and SEQ ID NO: 4) and SEQ ID NO: 5-6 (pair of primers SEQ ID NO: 5 and SEQ ID NO: 6).

The expression “the nucleic acid sequence selected from the group consisting of SEQ ID NO: x to SEQ ID NO: x+n” means “the nucleic acid sequence selected from the group consisting of SEQ ID NO: x, SEQ ID NO: x+1, SEQ ID NO: x+2 . . . and SEQ ID NO: x+n”. For example, the nucleic acid sequence selected from the group consisting of SEQ ID NO: 1 to SEQ ID NO: 6 means the nucleic acid sequence selected from the group consisting of SEQ ID NO: 1, SEQ ID NO: 2, SEQ ID NO: 3, SEQ ID NO: 4, SEQ ID NO: 5 and SEQ ID NO: 6.

Biological samples as referred herein include, without limitation, mammalian bodily fluids, especially oral fluids or scrapings, genital scrapings, in particular cervix scrapings.

Primers and amplicons encompassed by the invention are not limited to the sequences defined in the primers and amplicons depicted below. Primers and amplicons may encompass primers having at least 95% of identity with the primers and amplicons defined below. Primers can also comprise extra bases at the 5′ end. Also, primers shall be understood as embracing shorter sequences of at least 12, 15, 20 or 25 consecutive bases of the primers featured below. In some embodiments, it shall be understood that the invention also contemplates generic probes which have the sequences of the primers depicted herein and which are directly or indirectly labeled. The probes and primers can be extended or swifted from 1 to 15 bases depending on the desired specificity of the PCR amplification step and/or on the specificity of the detection step using standard parameters such as the nucleic acid size and GC contents, stringent hybridization conditions and temperature reactions. For example, low stringency conditions are used when it is desired to obtain broad positive results on a range of homologous targets whereas high stringency conditions are preferred to obtain positive results only if the specific target nucleic is present in the sample.

As used herein, the term “stringent hybridization conditions” refers to conditions under which the primer or probe will hybridize only to that exactly complementary target(s). The hybridization conditions affect the stability of hybrids, e.g., temperature, salt concentration, pH, formamide concentration and the like. These conditions are optimized to maximize specific binding and minimize non-specific binding of primer or probe to its target nucleic acid sequence. Stringent conditions are sequence dependent and will be different in different circumstances. Longer sequences hybridize specifically at higher temperatures. Generally, stringent conditions are selected to be about 5° C. lower than the thermal melting point (Tm) for the specific sequences at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+, typically about 0.01 to 1.0 M Na+ concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions include hybridization with a buffer solution of 20-30% formamide, 1 M NaCl, 1% SDS at 37° C. and a wash in 2*SSC at 40° C. Exemplary high stringency conditions include hybridization in 40-50% formamide, 1 M NaCl, 1% SDS at 37° C., and a wash in 0.1*SSC at 60° C. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002.

Moreover, to improve the hybridization with the coupled oligonucleotide, it can be advantageous for the oligonucleotide to contain an “arm” and a “spacer” sequence of bases. The use of an arm makes it possible, in effect, to bind the primer at a chosen distance from the support, enabling its conditions of interaction with the DNA to be improved. The arm advantageously consists of a linear carbon chain, comprising 1 to 18 and preferably 6 or 12 (CH₂) groups, and an amine which permits binding to the column. The arm is linked to a phosphate of the oligonucleotide or of a “spacer” composed of bases which do not interfere with the hybridization. Thus, the “spacer” can comprise purine bases. As an example, the “spacer” can comprise the sequence GAGG. The arm is advantageously composed of a linear carbon chain comprising 6 or 12 carbon atoms.

For implementation of the present invention, different types of support may be used. These can be functionalized chromatographic supports, in bulk or prepacked in a column, functionalized plastic surfaces or functionalized latex beads, magnetic or otherwise. Chromatographic supports are preferably used. As an example, the chromatographic supports capable of being used are agarose, acrylamide or dextran as well as their derivatives (such as Sephadex, Sepharose, Superose, etc.), polymers such as poly(styrene/divinylbenzene), or grafted or ungrafted silica, for example. The chromatography columns can operate in the diffusion or perfusion mode.

As used herein, the term “sequencing” is used in a broad sense and refers to any technique known by the skilled person including but not limited to Sanger dideoxy termination sequencing, whole-genome sequencing, sequencing by hybridization, pyrosequencing, capillary electrophoresis, cycle sequencing, single-base extension sequencing, solid-phase sequencing, high-throughput sequencing, massively parallel signature sequencing (MPSS), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD sequencing, MS-PET sequencing, mass spectrometry, and a combination thereof. In specific embodiments, the method and kit of the invention is adapted to run on ABI PRISM® 377 DNA Sequencer, an ABI PRISM® 310, 3100, 3100-Avant, 3730, or 3730×1 Genetic Analyzer, an ABI PRISM® 3700 DNA Analyzer, or an Applied Biosystems SOLiD™ System (all from Applied Biosystems), a Genome Sequencer 20 System (Roche Applied Science).

For all technologies described herein, although the said primers can be used in solution, in another embodiment the said primers are linked to a solid support.

To permit its covalent coupling to the support, the primer is generally functionalized. Thus, it may be modified by a thiol, amine or carboxyl terminal group at the 5′ or 3′ position. In particular, the addition of a thiol, amine or carboxyl group makes it possible, for example, to couple the oligonucleotide to a support bearing disulphide, maleimide, amine, carboxyl, ester, epoxide, cyanogen bromide or aldehyde functions. These couplings form by establishment of disulphide, thioether, ester, amide or amine links between the primer and the support. Any other method known to a person skilled in the art may be used, such as bifunctional coupling reagents, for example.

Moreover, to improve the hybridization with the coupled oligonucleotide, it can be advantageous for the oligonucleotide to contain an “arm” and a “spacer” sequence of bases. The use of an arm makes it possible, in effect, to bind the primer at a chosen distance from the support, enabling its conditions of interaction with the DNA to be improved. The arm advantageously consists of a linear carbon chain, comprising 1 to 18 and preferably 6 or 12 (CH₂) groups, and an amine which permits binding to the column. The arm is linked to a phosphate of the oligonucleotide or of a “spacer” composed of bases which do not interfere with the hybridization. Thus, the “spacer” can comprise purine bases. As an example, the “spacer” can comprise the sequence GAGG. The arm is advantageously composed of a linear carbon chain comprising 6 or 12 carbon atoms.

For implementation of the present invention, different types of support may be used. These can be functionalized chromatographic supports, in bulk or prepacked in a column, functionalized plastic surfaces or functionalized latex beads, magnetic or otherwise. Chromatographic supports are preferably used. As an example, the chromatographic supports capable of being used are agarose, acrylamide or dextran as well as their derivatives (such as Sephadex, Sepharose, Superose, etc.), polymers such as poly(styrene/divinylbenzene), or grafted or ungrafted silica, for example. The chromatography columns can operate in the diffusion or perfusion mode.

Composition of Primers for Detecting High Grade Squamous Intraepithelial Lesion

The present invention relates to a composition of primers for detecting HSIL comprising a first set of primers, called splice junctions set of primers, which comprises:
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a first subset of HPV16 specific pairs of primers having the nucleic acid sequence selected from the group consisting of SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26 and SEQ ID NO: 27-28; and
at least 2, at least 3, at least 4, at least 5, at least 6, at least 7, at least 8, at least 9, at least 10, at least 11, at least 12, at least 13, at least 14, at least 15, at least 16, at least 17 or at least 18, or 2, 3, 4, 5, 6, 7, 8, 9, 10, 11, 12, 13, 14, 15, 16, 17 or 18 pairs of primers of a second subset of HPV18 specific pairs of primers having the nucleic acid sequence selected from the group consisting of SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ ID NO: 43-44, SEQ ID NO: 45-46, SEQ ID NO: 47-48, SEQ ID NO: 49-50, SEQ ID NO: 51-52, SEQ ID NO: 53-54, SEQ ID NO: 55-56, SEQ ID NO: 57-58, SEQ ID NO: 59-60, SEQ ID NO: 61-62 and SEQ ID NO: 63-64; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a third subset of HPV31 specific pairs of primers having the nucleic acid sequence selected from the group consisting of SEQ ID NO: 65-66, SEQ ID NO: 67-68, SEQ ID NO: 69-70, SEQ ID NO: 71-72, SEQ ID NO: 73-74, SEQ ID NO: 75-76, SEQ ID NO: 77-78, SEQ ID NO: 79-80, SEQ ID NO: 81-82, SEQ ID NO: 83-84, SEQ ID NO: 85-86, SEQ ID NO: 87-88, SEQ ID NO: 89-90 and SEQ ID NO: 91-92; and
at least 2, at least 3, at least 4, at least 5, at least 6, at least 7, at least 8, at least 9, at least 10, at least 11, at least 12 or at least 13, or 2, 3, 4, 5, 6, 7, 8, 9, 10, 11, 12 or 13 pairs of primers of a fourth subset of HPV33 specific pairs of primers having the nucleic acid sequence selected from the group consisting of SEQ ID NO: 93-94, SEQ ID NO: 95-96, SEQ ID NO: 97-98, SEQ ID NO: 99-100, SEQ ID NO: 101-102, SEQ ID NO: 103-104, SEQ ID NO: 105-106, SEQ ID NO: 107-108, SEQ ID NO: 109-110, SEQ ID NO: 111-112, SEQ ID NO: 113-114, SEQ ID NO: 115-116 and SEQ ID NO: 117-118; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a fifth subset of HPV35 specific pairs of primers having the nucleic acid sequence selected from the group consisting of SEQ ID NO: 119-120, SEQ ID NO: 121-122, SEQ ID NO: 123-124, SEQ ID NO: 125-126, SEQ ID NO: 127-128, SEQ ID NO: 129-130, SEQ ID NO: 131-132, SEQ ID NO: 133-134, SEQ ID NO: 135-136, SEQ ID NO: 137-138, SEQ ID NO: 139-140, SEQ ID NO: 141-142, SEQ ID NO: 143-144 and SEQ ID NO: 145-146; and
at least 2, at least 3, at least 4, at least 5, at least 6, at least 7, at least 8, at least 9 or at least 10, or 2, 3, 4, 5, 6, 7, 8, 9 or 10 pairs of primers of a sixth subset of HPV39 specific pairs of primers having the nucleic acid sequence selected from the group consisting of SEQ ID NO: 147-148, SEQ ID NO: 149-150, SEQ ID NO: 151-152, SEQ ID NO: 153-154, SEQ ID NO: 155-156, SEQ ID NO: 157-158, SEQ ID NO: 159-160, SEQ ID NO: 161-162, SEQ ID NO: 163-164 and SEQ ID NO: 165-166; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13, or 14 pairs of primers of a seventh subset of HPV45 specific pairs of primers having the nucleic acid sequence selected from the group consisting of SEQ ID NO: 167-168, SEQ ID NO: 169-170, SEQ ID NO: 171-172, SEQ ID NO: 173-174, SEQ ID NO: 175-176, SEQ ID NO: 177-178, SEQ ID NO: 179-180, SEQ ID NO: 181-182, SEQ ID NO: 183-184, SEQ ID NO: 185-186, SEQ ID NO: 187-188, SEQ ID NO: 189-190, SEQ ID NO: 191-192 and SEQ ID NO: 193-194; and
at least 2, at least 3, at least 4, at least 5, at least 6, at least 7, at least 8, at least 9 or at least 10, or 2, 3, 4, 5, 6, 7, 8, 9 or 10 pairs of primers of an eighth subset of HPV51 specific pairs of primers having the nucleic acid sequence selected from the group consisting of SEQ ID NO: 195-196, SEQ ID NO: 197-198, SEQ ID NO: 199-200; SEQ ID NO: 201-202, SEQ ID NO:203-204, SEQ ID NO: 205-206, SEQ ID NO: 207-208, SEQ ID NO: 209-210, SEQ ID NO: 211-212 and SEQ ID NO: 213-214; and
at least 2, at least 3, at least 4, at least 5, at least 6, at least 7, at least 8, at least 9, at least 10, at least 11, at least 12, at least 13, at least 14, at least 15 or at least 16, or 2, 3, 4, 5, 6, 7, 8, 9, 10, 11, 12, 13, 14, 15 or 16 pairs of primers of a ninth subset of HPV52 specific pairs of primers having the nucleic acid sequence selected from the group consisting of SEQ ID NO: 215-216, SEQ ID NO: 217-218, SEQ ID NO: 219-220, SEQ ID NO: 221-222, SEQ ID NO: 223-224, SEQ ID NO: 225-226, SEQ ID NO: 227-228, SEQ ID NO: 229-230, SEQ ID NO: 231-232, SEQ ID NO: 233-234, SEQ ID NO: 235-236, SEQ ID NO: 237-238, SEQ ID NO: 239-240 SEQ ID NO: 241-242, SEQ ID NO: 243-244 and SEQ ID NO: 245-246; and
at least 2, at least 3, at least 4, at least 5, at least 6, at least 7, at least 8, at least 9, at least 10, at least 11, at least 12, at least 13, at least 14, at least 15 or at least 16, or 2, 3, 4, 5, 6, 7, 8, 9, 10, 11, 12, 13, 14, 15 or 16 pairs of primers of a tenth subset of HPV56 specific pairs of primers having the nucleic acid sequence selected from the group consisting of SEQ ID NO: 247-248, SEQ ID NO: 249-250, SEQ ID NO: 251-252, SEQ ID NO: 253-254, SEQ ID NO: 255-256, SEQ ID NO: 257-258, SEQ ID NO: 259-260, SEQ ID NO: 261-262, SEQ ID NO: 263-264, SEQ ID NO: 265-266, SEQ ID NO: 267-268, SEQ ID NO: 269-270, SEQ ID NO: 271-272, SEQ ID NO: 273-274, SEQ ID NO: 275-276 and SEQ ID NO: 277-278; and
at least 2, at least 3, at least 4, at least 5, at least 6, at least 7, at least 8, at least 9, at least 10, at least 11, at least 12 or at least 13, or 2, 3, 4, 5, 6, 7, 8, 9, 10, 11, 12 or 13 pairs of primers of an eleventh subset of HPV58 specific pairs of primers having the nucleic acid sequence selected from the group consisting of SEQ ID NO: 279-280, SEQ ID NO: 281-282, SEQ ID NO: 283-284, SEQ ID NO: 285-286, SEQ ID NO: 287-288, SEQ ID NO: 289-290, SEQ ID NO: 291-292, SEQ ID NO: 293-294, SEQ ID NO: 295-296, SEQ ID NO: 297-298, SEQ ID NO: 299-300, SEQ ID NO: 301-302 and SEQ ID NO: 303-304; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a twelfth subset of HPV59 specific pairs of primers having the nucleic acid sequence selected from the group consisting of SEQ ID NO: 305-306, SEQ ID NO: 307-308, SEQ ID NO: 309-310, SEQ ID NO: 311-312, SEQ ID NO: 313-314, SEQ ID NO: 315-316, SEQ ID NO: 317-318, SEQ ID NO: 319-320, SEQ ID NO: 321-322, SEQ ID NO: 323-324, SEQ ID NO: 325-326, SEQ ID NO: 327-328, SEQ ID NO: 329-330 and SEQ ID NO: 331-332; and
at least 2, at least 3, at least 4, at least 5, at least 6, at least 7, at least 8, at least 9, at least 10, at least 11, at least 12, at least 13, at least 14 or at least 15, or 2, 3, 4, 5, 6, 7, 8, 9, 10, 11, 12, 13, 14 or 15 pairs of primers of a thirteenth subset of HPV66 specific pairs of primers having the nucleic acid sequence selected from the group consisting of SEQ ID NO: 333-334, SEQ ID NO: 335-336, SEQ ID NO: 337-338, SEQ ID NO: 339-340, SEQ ID NO: 341-342, SEQ ID NO: 343-344, SEQ ID NO: 345-346, SEQ ID NO: 347-348, SEQ ID NO: 349-350, SEQ ID NO: 351-352, SEQ ID NO: 353-354, SEQ ID NO: 355-356, SEQ ID NO: 357-358, SEQ ID NO: 359-360 and SEQ ID NO: 361-362.

The splice junctions set of primers may further comprise:
at least 2, at least 3, at least 4, at least 5, at least 6, at least 7, at least 8, at least 9 or at least 10, or 2, 3, 4, 5, 6, 7, 8, 9 or 10 pairs of primers of a fourteenth subset of HPV68 specific pairs of primers having the nucleic acid sequence selected from the group consisting of SEQ ID NO: 363-364, SEQ ID NO: 365-366, SEQ ID NO: 367-368, SEQ ID NO: 369-370, SEQ ID NO: 371-372, SEQ ID NO: 373-374, SEQ ID NO: 375-376, SEQ ID NO: 377-378, SEQ ID NO: 379-380 and SEQ ID NO: 381-382; and/or
at least 2, at least 3, at least 4, at least 5, at least 6, at least 7, at least 8, at least 9, at least 10, at least 11, at least 12 or at least 13, or 2, 3, 4, 5, 6, 7, 8, 9, 10, 11, 12 or 13 pairs of primers of a fifteenth subset of HPV73 specific pairs of primers having the nucleic acid sequence selected from the group consisting of SEQ ID NO: 383-384, SEQ ID NO: 385-386, SEQ ID NO: 387-388, SEQ ID NO: 389-390 SEQ ID NO: 391-392, SEQ ID NO: 393-394, SEQ ID NO: 395-396, SEQ ID NO: 397-398, SEQ ID NO: 399-400, SEQ ID NO: 401-402, SEQ ID NO: 403-404, SEQ ID NO: 405-406 and SEQ ID NO: 407-408; and/or
at least 2, at least 3, at least 4, at least 5, at least 6, at least 7, at least 8, at least 9, at least 10 or at least 11, or 2, 3, 4, 5, 6, 7, 8, 9, 10 or 11 pairs of primers of a sixteenth subset of HPV82 specific pairs of primers having the nucleic acid sequence selected from the group consisting of SEQ ID NO: 409-410, SEQ ID NO: 411-412, SEQ ID NO: 413-414, SEQ ID NO: 415-416, SEQ ID NO: 417-418, SEQ ID NO: 419-420, SEQ ID NO: 421-422, SEQ ID NO: 423-424, SEQ ID NO: 425-426, SEQ ID NO: 427-428 and SEQ ID NO: 429-430.

These additional subsets of pairs of primers correspond to the putative high risk HPV: HPV68, HPV73 and HPV82.

The composition of primers for detecting HSIL may comprises the splice junctions set comprising at least 10 pairs of primers of each of the first to the thirteenth subsets of pairs of primers of the splice junctions set of primers as defined above and optionally at least 10 pairs of primers of the fourteenth and/or the fifteenth and/or the sixteenth subsets of pairs of primers of the splice junctions set of primers as defined above.

The composition of primers for detecting HSIL may comprises the splice junctions set comprising 2, 3, 4, 5, 6, 7, 8, 9 or more preferably 10 pairs of primers of each of the first to the thirteenth subsets of pairs of primers of the splice junctions set of primers as defined above and optionally 2, 3, 4, 5, 6, 7, 8, 9 or more preferably 10 pairs of primers of the fourteenth and/or the fifteenth and/or the sixteenth subsets of pairs of primers of the splice junctions set of primers as defined above.

The composition of primers for detecting HSIL may comprises the splice junctions set consisting of 2, 3, 4, 5, 6, 7, 8, 9 or more preferably 10 pairs of primers of each of the first to the thirteenth subsets of pairs of primers of the splice junctions set of primers as defined above and optionally of 2, 3, 4, 5, 6, 7, 8, 9 or more preferably 10 pairs of primers of the fourteenth and/or the fifteenth and/or the sixteenth subsets of pairs of primers of the splice junctions set of primers as defined above.

In one embodiment, the composition of primers for detecting HSIL according to the invention comprises the pairs of primers having the nucleic acid sequence SEQ ID NO: 1-2 to SEQ ID NO: 361-362 and optionally of SEQ ID NO: 363-364 to SEQ ID NO: 381-382 and/or SEQ ID NO: 383-384 to SEQ ID NO: 385-386 and/or SEQ ID NO: 387-388 to SEQ ID NO: 429-430.

In a preferred embodiment, the composition of primers for detecting HSIL according to the invention comprises the pairs of primers having the nucleic acid sequence SEQ ID NO: 1-2 to SEQ ID NO: 429-430.

In one embodiment, the composition of primers for detecting HSIL consists of the pairs of primers having the nucleic acid sequence SEQ ID NO: 1-2 to SEQ ID NO: 361-362 and optionally of SEQ ID NO: 363-364 to SEQ ID NO: 381-382 and/or SEQ ID NO: 383-384 to SEQ ID NO: 385-386 and/or SEQ ID NO: 387-388 to SEQ ID NO: 429-430.

In a preferred embodiment, the composition of primers for detecting HSIL according to the invention consists of the pairs of primers having the nucleic acid sequence SEQ ID NO: 1-2 to SEQ ID NO: 429-430.

The splice junctions set of primers of the invention may be defined by the nucleic acid sequence of the pairs of primers that compose it as defined above or by the nucleic acid sequence of the amplicons which are produced by the pairs of primers that compose it as defined below.

The pairs of primers that compose splice junctions set of primers as defined above correspond to the amplicons which are produced by the pairs of primers that compose splice junctions set of primers as defined below. The correspondence between the pairs of primers and their corresponding amplicons is given in table 2Abis.

The present invention also relates to a composition of primers for detecting HSIL comprising a first set of pairs of primers, called splice junctions set of primers, which comprises:
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers a first subset of HPV16 specific pairs of primers able to produce the amplicons having the nucleic acid sequence selected from the group consisting of SEQ ID NO: 1501, SEQ ID NO: 1502, SEQ ID NO: 1503, SEQ ID NO: 1504, SEQ ID NO: 1505, SEQ ID NO: 1506, SEQ ID NO: 1507, SEQ ID NO: 1508, SEQ ID NO: 1509, SEQ ID NO: 1510, SEQ ID NO: 1511, SEQ ID NO: 1512, SEQ ID NO: 1513 and SEQ ID NO: 1514; and
at least 2, at least 3, at least 4, at least 5, at least 6, at least 7, at least 8, at least 9, at least 10, at least 11, at least 12, at least 13, at least 14, at least 15, at least 16, at least 17 or at least 18 or 2, 3, 4, 5, 6, 7, 8, 9, 10, 11, 12, 13, 14, 15, 16, 17 or 18 pairs of primers of a second subset of HPV18 specific pairs of primers able to produce the amplicons having the nucleic acid sequence selected from the group consisting of SEQ ID NO: 1515, SEQ ID NO: 1516, SEQ ID NO: 1517, SEQ ID NO: 1518, SEQ ID NO: 1519, SEQ ID NO: 1520, SEQ ID NO: 1521, SEQ ID NO: 1522, SEQ ID NO: 1523, SEQ ID NO: 1524, SEQ ID NO: 1525, SEQ ID NO: 1526, SEQ ID NO: 1527, SEQ ID NO: 1528, SEQ ID NO: 1529, SEQ ID NO: 1530, SEQ ID NO: 1531 and SEQ ID NO: 1532; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 of pairs primers of a third subset of HPV31 specific pairs of primers able to produce the amplicons having the nucleic acid sequence selected from the group consisting of SEQ ID NO: 1533, SEQ ID NO: 1534, SEQ ID NO: 1535, SEQ ID NO: 1536, SEQ ID NO: 1537, SEQ ID NO: 1538, SEQ ID NO: 1539, SEQ ID NO: 1540, SEQ ID NO: 1541, SEQ ID NO: 1542, SEQ ID NO: 1543, SEQ ID NO: 1544, SEQ ID NO: 154 and SEQ ID NO: 1546; and
at least 2, at least 3, at least 4, at least 5, at least 6, at least 7, at least 8, at least 9, at least 10, at least 11, at least 12 or at least 13, or 2, 3, 4, 5, 6, 7, 8, 9, 10, 11, 12 or 13 pairs of primers of a fourth subset of pairs of primers HPV33 specific able to produce the amplicons having the nucleic acid sequence selected from the group consisting of SEQ ID NO:1547, SEQ ID NO: 1548, SEQ ID NO: 1549, SEQ ID NO: 1550, SEQ ID NO: 1551, SEQ ID NO: 1552, SEQ ID NO: 1553, SEQ ID NO: 1554, SEQ ID NO: 1555, SEQ ID NO: 1556, SEQ ID NO: 1557, SEQ ID NO: 1558 and SEQ ID NO:1559; and
at least 2, at least 3, at least 4, at least 5, at least 6, at least 7, at least 8, at least 9, at least 10, at least 11, at least 12, at least 13, or at least 14 or 2, 3, 4, 5, 6, 7, 8, 9, 10, 11, 12, 13 or 14 pairs of primers of a fifth subset of HPV35 specific pairs of primers able to produce the amplicons having the nucleic acid sequence selected from the group consisting of SEQ ID NO: 1560, SEQ ID NO: 1561, SEQ ID NO: 1562, SEQ ID NO: 1563, SEQ ID NO: 1564, SEQ ID NO: 1565, SEQ ID NO: 1566, SEQ ID NO: 1567, SEQ ID NO: 1568, SEQ ID NO: 1569, SEQ ID NO: 1570, SEQ ID NO: 1571, SEQ ID NO: 1572 and SEQ ID NO: 1573; and
at least 2, at least 3, at least 4, at least 5, at least 6, at least 7, at least 8, at least 9 or at least 10, or 2, 3, 4, 5, 6, 7, 8, 9 or 10 pairs of primers of a sixth subset of HPV39 specific pairs of primers able to produce the amplicons having the nucleic acid sequence selected from the group consisting of SEQ ID NO: 1574, SEQ ID NO: 1575, SEQ ID NO: 1576, SEQ ID NO: 1577, SEQ ID NO: 1578, SEQ ID NO: 1579, SEQ ID NO: 1580, SEQ ID NO: 1581, SEQ ID NO: 1582 and SEQ ID NO: 1583; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a seventh subset of HPV45 specific pairs of primers able to produce the amplicons having the nucleic acid sequence selected from the group consisting of SEQ ID NO: 1584, SEQ ID NO: 1585, SEQ ID NO: 1586, SEQ ID NO: 1587, SEQ ID NO: 1588, SEQ ID NO: 1589, SEQ ID NO: 1590, SEQ ID NO: 1591, SEQ ID NO: 1592, SEQ ID NO: 1593, SEQ ID NO: 1594, SEQ ID NO: 1595, SEQ ID NO: 1596 and SEQ ID NO: 1597; and
at least 2, at least 3, at least 4, at least 5, at least 6, at least 7, at least 8, at least 9 or at least 10, or 2, 3, 4, 5, 6, 7, 8, 9 or 10 pairs of primers of an eighth subset of HPV51 specific pairs of primers able to produce the amplicons having the nucleic acid sequence selected from the group consisting of SEQ ID NO: 1598, SEQ ID NO: 1599, SEQ ID NO: 1600, SEQ ID NO: 1601, SEQ ID NO: 1602, SEQ ID NO: 1603, SEQ ID NO: 1604, SEQ ID NO: 1605, SEQ ID NO: 1606 and SEQ ID NO: 1607; and
at least 2, at least 3, at least 4, at least 5, at least 6, at least 7, at least 8, at least 9, at least 10, at least 11, at least 12, at least 13, at least 14, at least 15 or at least 16, or 2, 3, 4, 5, 6, 7, 8, 9, 10, 11, 12, 13, 14, 15 or 16 pairs of primers a ninth subset of HPV52 specific pairs of primers able to produce the amplicons having the nucleic acid sequence selected from the group consisting of SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1616, SEQ ID NO: 1617, SEQ ID NO: 1618, SEQ ID NO: 1619, SEQ ID NO: 1620, SEQ ID NO: 1621, SEQ ID NO: 1622 and SEQ ID NO: 1623; and
at least 2, at least 3, at least 4, at least 5, at least 6, at least 7, at least 8, at least 9, at least 10, at least 11, at least 12, at least 13, at least 14, at least 15 or at least 16, or 2, 3, 4, 5, 6, 7, 8, 9, 10, 11, 12, 13, 14, 15 or 16 pairs of primers of a tenth subset of HPV56 specific pairs of primers able to produce the amplicons having the nucleic acid sequence selected from the group consisting of SEQ ID NO: 1624, SEQ ID NO: 1625, SEQ ID NO: 1626, SEQ ID NO: 1627, SEQ ID NO: 1628, SEQ ID NO: 1629, SEQ ID NO: 1630, SEQ ID NO: 1631, SEQ ID NO: 1632, SEQ ID NO: 1633, SEQ ID NO: 1634, SEQ ID NO: 1635, SEQ ID NO: 1636, SEQ ID NO: 1637, SEQ ID NO: 1638 and SEQ ID NO: 1639; and
at least 2, at least 3, at least 4, at least 5, at least 6, at least 7, at least 8, at least 9, at least 10, at least 11, at least 12 or at least 13, or 2, 3, 4, 5, 6, 7, 8, 9, 10, 11, 12 or 13 pairs of primers of an eleventh subset of HPV58 specific pairs of primers able to produce the amplicons having the nucleic acid sequence selected from the group consisting of SEQ ID NO: 1640, SEQ ID NO: 1641, SEQ ID NO: 1642, SEQ ID NO: 1643, SEQ ID NO: 1644, SEQ ID NO: 1645, SEQ ID NO: 1646, SEQ ID NO: 1647, SEQ ID NO: 1648, SEQ ID NO: 1649, SEQ ID NO: 1650, SEQ ID NO: 1651 and SEQ ID NO: 1652; and
at least 2, at least 3, at least 4, at least 5, at least 6, at least 7, at least 8, at least 9, at least 10, at least 11, at least 12, at least 13 or at least 14, or 2, 3, 4, 5, 6, 7, 8, 9, 10, 11, 12, 13 or 14 pairs of primers of a twelfth subset of HPV59 specific pairs of primers able to produce the amplicons having the nucleic acid sequence selected from the group consisting of SEQ ID NO: 1653, SEQ ID NO: 1654, SEQ ID NO: 1655, SEQ ID NO: 1656, SEQ ID NO: 1657, SEQ ID NO: 1658, SEQ ID NO: 1659, SEQ ID NO: 1660, SEQ ID NO: 1661, SEQ ID NO: 1662, SEQ ID NO: 1663, SEQ ID NO: 1664, SEQ ID NO: 1665 and SEQ ID NO: 1666; and
at least 2, at least 3, at least 4, at least 5, at least 6, at least 7, at least 8, at least 9, at least 10, at least 11, at least 12, at least 13, at least 14 or at least 15, or 2, 3, 4, 5, 6, 7, 8, 9, 10, 11, 12, 13, 14 or 15 pairs of primers a thirteenth subset of HPV66 specific pairs of primers able to produce the amplicons having the nucleic acid sequence selected from the group consisting of SEQ ID NO: 1667, SEQ ID NO: 1668, SEQ ID NO: 1669, SEQ ID NO: 1670, SEQ ID NO: 1671, SEQ ID NO: 1672, SEQ ID NO: 1673, SEQ ID NO: 1674, SEQ ID NO: 1675, SEQ ID NO: 1676, SEQ ID NO: 1677, SEQ ID NO: 1678, SEQ ID NO: 1679, SEQ ID NO: 1680 and SEQ ID NO: 1681.

The splice junctions set of primers may further comprise:
at least 2, at least 3, at least 4, at least 5, at least 6, at least 7, at least 8, at least 9 or at least 10, or 2, 3, 4, 5, 6, 7, 8, 9 or 10 pairs of primers of a fourteenth subset of HPV68 specific pairs of primers able to produce the amplicons having the nucleic acid sequence selected from the group consisting of SEQ ID NO: 1682, SEQ ID NO: 1683, SEQ ID NO: 1684, SEQ ID NO: 1685, SEQ ID NO: 1686, SEQ ID NO: 1687, SEQ ID NO: 1688, SEQ ID NO: 1689, SEQ ID NO: 1690 and SEQ ID NO: 1691; and/or
at least 2, at least 3, at least 4, at least 5, at least 6, at least 7, at least 8, at least 9, at least 10, at least 11, at least 12 or at least 13, at least 14, at least 15, at least 16, at least 17 or at least 18, or 2, 3, 4, 5, 6, 7, 8, 9, 10, 11, 12, 13, 14, 15, 16, 17 or 18 pairs of primers of a fifteenth subset of HPV73 specific pairs of primers able to produce the amplicons having the nucleic acid sequence selected from the group consisting of SEQ ID NO: 1692, SEQ ID NO: 1693, SEQ ID NO: 1694, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703 and SEQ ID NO: 1704; and/or
at least 2, at least 3, at least 4, at least 5, at least 6, at least 7, at least 8, at least 9, at least 10 or at least 11, or 2, 3, 4, 5, 6, 7, 8, 9, 10 or 11 pairs of primers of a sixteenth subset of HPV82 specific pairs of primers able to produce the amplicons having the nucleic acid sequence selected from the group consisting of SEQ ID NO: 1705, SEQ ID NO: 1706, SEQ ID NO: 1707, SEQ ID NO: 1708, SEQ ID NO: 1709, SEQ ID NO: 1710, SEQ ID NO: 1711, SEQ ID NO: 1712, SEQ ID NO: 1713, SEQ ID NO: 1714 and SEQ ID NO: 1715.

The composition of primers for detecting HSIL may comprises the splice junction set of primers comprising at least 10 pairs of primers of each of the first to the thirteenth subsets of pairs of primers of the splice junctions set of primers as defined above and optionally at least 10 pairs of primers of the fourteenth and/or the fifteenth and/or the sixteenth subsets of pairs of primers of the splice junctions set of primers as defined above.

The composition of primers for detecting HSIL may comprises the splice junction set of primers comprising 2, 3, 4, 5, 6, 7, 8, 9 or more preferably 10 pairs of primers of each of the first to the thirteenth subsets of pairs of primers of the splice junctions set of primers as defined above and optionally 2, 3, 4, 5, 6, 7, 8, 9 or more preferably 10 pairs of primers of the fourteenth and/or the fifteenth and/or the sixteenth subsets of pairs of primers of the splice junctions set of primers as defined above.

The composition of primers for detecting HSIL may comprises the splice junction set of primers consisting of 2, 3, 4, 5, 6, 7, 8, 9 or more preferably 10 pairs of primers of each of the first to the thirteenth subsets of pairs of primers of the splice junctions set of primers as defined above and of optionally 2, 3, 4, 5, 6, 7, 8, 9 or more preferably 10 pairs of primers of the fourteenth and/or the fifteenth and/or the sixteenth subsets of pairs of primers of the splice junctions set of primers as defined above.

In one embodiment, the composition for detecting HSIL of primers according to the invention comprises the splice junction set of primers comprising the pairs of primers able to produce the amplicons having the nucleic acid sequence SEQ ID NO: 1501 to SEQ ID NO: 1681 and optionally of SEQ ID NO: 1682 to SEQ ID NO: 1691 and/or SEQ ID NO: 1692 to SEQ ID NO: 1704 and/or SEQ ID NO: 1705 to SEQ ID NO: 1715.

In a preferred embodiment, the composition of primers for detecting HSIL according to the invention comprises the splice junction set of primers comprising the pairs of primers able to produce the amplicons having the nucleic acid sequence SEQ ID NO: 1501 to SEQ ID NO: 1715.

In one embodiment, the composition of primers consists of the splice junction set of primers consisting of the pairs of primers having the nucleic acid sequence SEQ ID NO: 1501 to SEQ ID NO: 1681 and optionally of SEQ ID NO: 1682 to SEQ ID NO: 1691 and/or SEQ ID NO: 1692 to SEQ ID NO: 1704 and/or SEQ ID NO: 1705 to SEQ ID NO: 1715.

In a preferred embodiment, the composition of primers for detecting HSIL according to the invention consists of the splice junction set of primers consisting of the pairs of primers able to produce the amplicons having the nucleic acid sequence SEQ ID NO: 1501 to SEQ ID NO: 1715.

In one embodiment, the composition of primers for detecting HSIL does not comprise an additional set of primers selected from the group consisting of a unsplice junctions set of primers, a genomic set of primers and a fusion set of primers. In particular, the composition of primers for detecting HSIL may not comprise an unsplice junctions set of primers, a genomic set of primers and a fusion set of primers.

In one embodiment, the composition of primers for detecting HSIL comprises a human set of primers. The primers of the human set of primers target human sequences.

The human set of primers may be used as an internal control.

The human set of primers may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30, or 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 pairs of primers having the nucleic acid sequence selected from the group consisting of SEQ ID NO: 1441-1442, SEQ ID NO: 1443-1444, SEQ ID NO: 1445-1446, SEQ ID NO: 1447-1448, SEQ ID NO: 1449-1450, SEQ ID NO: 1451-1452, SEQ ID NO: 1453-1454, SEQ ID NO: 1455-1456, SEQ ID NO: 1457-1458, SEQ ID NO: 1459-1460, SEQ ID NO: 1461-1462, SEQ ID NO: 1463-1464, SEQ ID NO: 1465-1466, SEQ ID NO: 1467-1468, SEQ ID NO: 1469-1470, SEQ ID NO: 1471-1472, SEQ ID NO: 1473-1474, SEQ ID NO: 1475-1476, SEQ ID NO: 1477-1478, SEQ ID NO: 1479-1480, SEQ ID NO: 1481-1482, SEQ ID NO: 1483-1484, SEQ ID NO: 1485-1486, SEQ ID NO: 1487-1488, SEQ ID NO: 1489-1490, SEQ ID NO: 1491-1492, SEQ ID NO: 1493-1494, SEQ ID NO: 1495-1496, SEQ ID NO: 1497-1498 and SEQ ID NO: 1499-1500.

In one preferred embodiment, the human set of primers comprises SEQ ID NO: 1441-1442 to SEQ ID NO: 1499-1500.

In one more preferred embodiment, the human set of primers consists of SEQ ID NO: 1441-1442 to SEQ ID NO: 1499-1500.

The human set of primers may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30, or 2, 3, 4, 5, 6, 7, 8, 9, 10, 11, 12, 13, 14, 15, 16, 17, 18, 19, 20, 21, 22, 23, 24, 25, 26, 27, 28, 29 or 30 pairs of primers able to produce the amplicons having the nucleic acid sequence selected from the group consisting of SEQ ID NO: 2221 to SEQ ID NO: 2250.

In one preferred embodiment, the human set of primers comprises pairs of primers able to produce the amplicons having the nucleic acid sequence SEQ ID NO: 2221 to SEQ ID NO: 2250.

In one more preferred embodiment, the human set of primers consists of pairs of primers able to produce the amplicons having the nucleic acid sequence SEQ ID NO: 2221 to SEQ ID NO: 2250.

In one embodiment, the composition of primers for detecting HSIL may also comprise a fusion set of primers. The primers of fusion set of primers target high risk and optionally of putative high risk HPV fusion transcripts.

The primers of fusion set of primers may comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a first subset of HPV16 specific pairs of primers having the nucleic acid sequence selected from the group consisting of SEQ ID NO: 865-866 to SEQ ID NO: 899-900;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a second subset of HPV18 specific pairs of primers having the nucleic acid sequence selected from the group consisting of SEQ ID NO: 901-902 to SEQ ID NO: 935-936;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a third subset of HPV31 specific pairs of primers having the nucleic acid sequence selected from the group consisting of SEQ ID NO: 937-938 to SEQ ID NO: 971-972;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourth subset of HPV33 specific pairs of primers having the nucleic acid sequence selected from the group consisting of SEQ ID NO: 973-974 to SEQ ID NO: 1007-1008;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifth subset of HPV35 specific pairs of primers having the nucleic acid sequence selected from the group consisting of SEQ ID NO: 1009-1010 to SEQ ID NO: 1043-1044;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixth subset of HPV39 specific pairs of primers having the nucleic acid sequence selected from the group consisting of SEQ ID NO: 1045-1046 to SEQ ID NO: 1079-1080;
at least 1, at least 2, at least 3, at least 4, at least 5, at least 6, at least 7, at least 8, at least 9, at least 10, at least 11, at least 12, at least 13, at least 14, at least 15, at least 16, preferably at least 17, more preferably 18 or 1, 2, 3, 4, 5, 6, 7, 8, 9, 10, 11, 12, 13, 14, 15, 16, 17 or 18 pairs of primers of a seventh subset of HPV45 specific pairs of primers having the nucleic acid sequence selected from the group consisting of SEQ ID NO: 1081-1082 to SEQ ID NO: 1115-1116.
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a eighth subset of HPV51 specific pairs of primers having the nucleic acid sequence selected from the group consisting of SEQ ID NO: 1117-1118 to SEQ ID NO: 1151-1152;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a ninth subset of HPV52 specific pairs of primers having the nucleic acid sequence selected from the group consisting of SEQ ID NO: 1153-1154 to SEQ ID NO: 1187-1188;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a tenth subset of HPV56 specific pairs of primers having the nucleic acid sequence selected from the group consisting of SEQ ID NO: 1189-1190 to SEQ ID NO: 1223-1224;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of an eleventh subset of HPV58 specific pairs of primers having the nucleic acid sequence selected from the group consisting of SEQ ID NO: 1225-1226 to SEQ ID NO: 1259-1260;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a twelfth subset of HPV59 specific pairs of primers having the nucleic acid sequence selected from the group consisting of SEQ ID NO: 1261-1262 to SEQ ID NO: 1295-1296;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a thirteenth subset of HPV66 specific pairs of primers having the nucleic acid sequence selected from the group consisting of SEQ ID NO: 1297-1298 to SEQ ID NO: 1331-1332.

The fusion set of primers may further comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourteenth subset of HPV68 specific pairs of primers having the nucleic acid sequence selected from the group consisting of SEQ ID NO: 1333-1334 to SEQ ID NO: 1367-1368;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifteenth subset of HPV73 specific pairs of primers having the nucleic acid sequence selected from the group consisting of SEQ ID NO:1369-1370 to SEQ ID NO: 1403-1404;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixteenth subset of HPV82 specific pairs of primers having the nucleic acid sequence selected from the group consisting of SEQ ID NO: 1405-1406 to SEQ ID NO: 1439-1440.

In one embodiment, the fusion set of primers comprises the pairs of primers having the nucleic acid sequence SEQ ID NO: 865-866 to SEQ ID NO: 1331-1332 and optionally of SEQ ID NO: 1333-1334 to SEQ ID NO: 1367-1368 and/or SEQ ID NO: 1369-1370 to SEQ ID NO: 1403-1404 and/or SEQ ID NO: 1405-1406 to SEQ ID NO: 1439-1440.

In a preferred embodiment, the fusion set of primers comprises the pairs of primers having the nucleic acid sequence SEQ ID NO: 865-866 to SEQ ID NO: 1439-1440.

In one embodiment, the fusion set of primers consists of the pairs of primers having the nucleic acid sequence SEQ ID NO: 865-866 to SEQ ID NO: 1331-1332 and optionally of SEQ ID NO: 1333-1334 to SEQ ID NO: 1367-1368 and/or SEQ ID NO: 1369-1370 to SEQ ID NO: 1403-1404 and/or SEQ ID NO: 1405-1406 to SEQ ID NO: 1439-1440.

In a preferred embodiment, the fusion set of primers consists of the pairs of primers having the nucleic acid sequence SEQ ID NO: 865-866 to SEQ ID NO: 1439-1440.

The primers of fusion set of primers may comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a first subset of HPV16 specific pairs of primers able to produce the amplicons having the nucleic acid sequence selected from the group consisting of SEQ ID NO: 1933 to 1 SEQ ID NO: 1950;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a second subset of HPV18 specific pairs of primers able to produce the amplicons having the nucleic acid sequence selected from the group consisting of SEQ ID NO: 1951 to SEQ ID NO: 1968;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a third subset of HPV31 specific pairs of primers able to produce the amplicons having the nucleic acid sequence selected from the group consisting of SEQ ID NO: 1969 to SEQ ID NO: 1986;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourth subset of HPV33 specific pairs of primers able to produce the amplicons having the nucleic acid sequence selected from the group consisting of SEQ ID NO: 1987 to SEQ ID NO: 2004;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifth subset of HPV35 specific pairs of primers able to produce the amplicons having the nucleic acid sequence selected from the group consisting of SEQ ID NO: 2005 to SEQ ID NO: 2022;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixth subset of HPV39 specific pairs of primers able to produce the amplicons having the nucleic acid sequence selected from the group consisting of SEQ ID NO: 2023 to SEQ ID NO: 2040;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a seventh subset of HPV45 specific pairs of primers selected from the group consisting of SEQ ID NO: 2041 to SEQ ID NO: 2058,
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a eighth subset of HPV51 specific pairs of primers able to produce the amplicons having the nucleic acid sequence selected from the group consisting of SEQ ID NO: 2059 to SEQ ID NO: 2076;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a ninth subset of HPV52 specific pairs of primers able to produce the amplicons having the nucleic acid sequence selected from the group consisting of SEQ ID NO: 2077 to SEQ ID NO: 2094;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a tenth subset of HPV56 specific pairs of primers able to produce the amplicons having the nucleic acid sequence selected from the group consisting of SEQ ID NO: 2095 to SEQ ID NO: 2112;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of an eleventh subset of HPV58 specific pairs of primers able to produce the amplicons having the nucleic acid sequence selected from the group consisting of SEQ ID NO: 2113 to SEQ ID NO: 2130;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a twelfth subset of HPV59 specific pairs of primers able to produce the amplicons having the nucleic acid sequence selected from the group consisting of SEQ ID NO: 2131 to SEQ ID NO: 2148;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a thirteenth subset of HPV66 specific pairs of primers able to produce the amplicons having the nucleic acid sequence selected from the group consisting of SEQ ID NO: 2149 to SEQ ID NO: 2166.

The fusion set may also comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourteenth subset of HPV68 specific pairs of primers able to produce the amplicons having the nucleic acid sequence selected from the group consisting of SEQ ID NO: 2167 to SEQ ID NO: 2184; and/or
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifteenth subset of HPV73 specific pairs of primers able to produce the amplicons having the nucleic acid sequence selected from the group consisting of SEQ ID NO:2185 to SEQ ID NO: 2202; and/or
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixteenth subset of HPV82 specific pairs of primers able to produce the amplicons having the nucleic acid sequence selected from the group consisting of SEQ ID NO: 2203 to SEQ ID NO: 2220.

In one embodiment, the fusion set of primers comprises the pairs of primers having the nucleic acid sequence SEQ ID NO: 1933 to SEQ ID NO: 2166 and optionally of SEQ ID NO: 2167 to SEQ ID NO: 2184 and/or SEQ ID NO: 2185 to SEQ ID NO: 2202 and/or SEQ ID NO: 2203 to SEQ ID NO: 2220.

In a preferred embodiment, the fusion set of primers comprises the pairs of primers able to produce the amplicons having the nucleic acid sequence SEQ ID NO: 1869 to SEQ ID NO: 2220.

In one embodiment, the fusion set of primers consists of the pairs of primers able to produce the amplicons having the nucleic acid sequence SEQ ID NO: 1933 to SEQ ID NO: 2166 and optionally of SEQ ID NO: 2167 to SEQ ID NO: 2184 and/or SEQ ID NO: 2185 to SEQ ID NO: 2202 and/or SEQ ID NO: 2203 to SEQ ID NO: 2220.

In a preferred embodiment, the fusion set of primers consists of the pairs of primers able to produce the amplicons having the nucleic acid sequence SEQ ID NO: 1869 to SEQ ID NO: 2220.

The present invention also relates to a kit for detecting HSIL comprising the composition of primers for detecting HSIL of the invention and optionally reagents for a cDNA amplification. Reagents available for this purpose are well-known in the art and include the DNA polymerases, buffers for the enzymes, detergents, enhancing agents. The kit of the invention may also comprise reagent for reverse transcription and/or for sequencing. In some preferred embodiments of the kit of the invention, the primers, and optional reagents are in lyophilised form to allow ambient storage. The components of the kits are packaged together into any of the various containers suitable for nucleic acid amplification such as plates, slides, wells, dishes, beads, particles, cups, strands, chips, strips and others. The kit optionally includes instructions for performing at least one specific embodiment of the method of the invention. In some advantageous embodiments, the kit comprises micro-well plates or microtubes, preferably in a dried format, i.e., wherein the wells of the plates or microtubes comprise a dried composition containing at least the primers, and preferably further comprising all the reagents for the reverse transcription, cDNA amplification or sequencing.

The present invention also relates to the use of the composition of primers for detecting HSIL of the invention or of the kit for detecting HSIL of the invention.

An In Vitro Method for Detecting HSIL in a Biological Sample

The present invention also relates to an in vitro method for detecting HSIL in a biological sample comprising the steps of:
(a) extraction of RNA from the biological sample,
(b) reverse transcription of the RNA so as to generate cDNA,
(c) amplification of the cDNA generated at step (b) with the composition of primers of the invention so as to produce amplicons,
(d) quantifying the expression level of each amplicon produced at step (c),
(e) determining if the biological sample comprises HSIL based on the expression level of the amplicons quantified at step (d).

Preferably, the step (d) of quantifying the expression level of each amplicon is carried by sequencing.

The step (d) of quantifying the expression level of each amplicon may comprise the steps of:
(d1) sequencing the amplicons so as to generate reads,
(d2) aligning the reads to sequence of the corresponding amplicon,
(d3) for each amplicon, quantifying the reads corresponding to the sequence of said amplicon.

The quantification of the expression level of each amplicons may be carried using a partial digestion of the amplicons. Then, the step (d) of quantifying the expression level of each amplicon may comprise the steps of:
(d1) partially digesting the amplicon so as to generate fragments,
(d2) sequencing the fragments produced at step (d1) so as to generate reads,
(d3) aligning the reads to sequence of the corresponding amplicon,
(d4) for each amplicon, quantifying the reads corresponding to the sequence of said amplicon.

In a preferred embodiment, the step of determining if the biological sample comprises HSIL comprises a step of determining if the biological sample comprises HSIL corresponding to one HPV type defined herein based on the expression level of the amplicons quantified in step (d) specific of the said HPV type. In this embodiment, the step of determining if the biological sample comprises HSIL is carried for each HPV type. Thus, for each HPV type, the expression level of the amplicons corresponding this HPV type is analyzed and it is determined if the biological sample comprises a HSIL corresponding to this HPV type. If it is determined that the biological sample comprises a HSIL corresponding to at least one HPV type, than the biological sample is classified as comprising HSIL.

Preferably, the step of determining if the biological sample comprises HSIL is carried out by using a logistic regression analysis wherein the variables depend on the quantified level of expression the amplicons.

Thus, the step of determining if the biological sample comprises HSIL may comprise:
for each type of HPV selected from the group consisting of HPV16, HPV18, HPV31, HPV33, HPV35, HPV39, HPV45, HPV51, HPV52, HPV56, HPV58, HPV59, and HPV66 and optionally HPV68 and/or HPV73 and/or HPV82, a step calculating a probability p_{HPVj} that the biological sample comprises an HSIL of HPVⱼ type wherein ⱼ=16, 18, 31, 33, 35, 39, 45, 51, 52, 56, 58, 59, 66, 68, 73 and/or 82 using the following formula:

$$logit ( {p}_{H P V j} ) = {β}_{0} + {\sum }_{i = 1}^{25} ( {β}_{i} {X}_{i j} )$$

with:
β₀ is the intercept,
βᵢ is a coefficient corresponding to a given splice junction, called splice junction i,
βᵢⱼ is a variable depending on the quantified level of expression of the amplicon corresponding to the splice junction i for the HPVⱼ

wherein if one p_{HPVj} is higher than 0.5, it is indicative of the presence of a HPVⱼ HSIL in the biological sample.

In a preferred embodiment, the amplicons corresponding to the splice junction i=1 are respectively:
the amplicons corresponding to the splice junction i=1 are respectively SEQ ID NO: 1501 for HPV16, SEQ ID NO: 1515 for HPV18, SEQ ID NO: 1533 for HPV31, SEQ ID NO: 1547 for HPV33, SEQ ID NO: 1560 for HPV35, SEQ ID NO: 1574 for HPV39, SEQ ID NO: 1584 for HPV45, SEQ ID NO: 1598 for HPV51, SEQ ID NO: 1608 for HPV52, SEQ ID NO: 1624 for HPV56, SEQ ID NO: 1640 for HPV58, SEQ ID NO: 1653 for HPV59, SEQ ID NO: 1667 for HPV66, SEQ ID NO: 1682 for HPV68, SEQ ID NO: 1692 for HPV73 and SEQ ID NO: 1705 for HPV82,
the amplicons corresponding to the splice junction i=2 are respectively SEQ ID NO: 1502 for HPV16, SEQ ID NO: 1516 for HPV18, SEQ ID NO: 1534 for HPV31, SEQ ID NO: 1548 for HPV33, SEQ ID NO: 1561 for HPV35, absent for HPV39, SEQ ID NO: 1585 for HPV45, absent for HPV51, SEQ ID NO: 1609 for HPV52, SEQ ID NO: 1625 for HPV56, SEQ ID NO: 1641 for HPV58, SEQ ID NO: 1654 for HPV59, SEQ ID NO: 1668 for HPV66, absent for HPV68, absent for HPV73 and absent for HPV82,
the amplicons corresponding to the splice junction i=3 are respectively SEQ ID NO: 1503 for HPV16, SEQ ID NO: 1517 for HPV18, SEQ ID NO: 1535 for HPV31, SEQ ID NO: 1549 for HPV33, SEQ ID NO: 1562 for HPV35, absent for HPV39, absent for HPV45, SEQ ID NO: 1599 for HPV51, SEQ ID NO: 1610 for HPV52, SEQ ID NO: 1626 for HPV56, SEQ ID NO: 1642 for HPV58, absent for HPV59, SEQ ID NO: 1669 for HPV66, SEQ ID NO: 1683 for HPV68, SEQ ID NO: 1693 for HPV73 and SEQ ID NO: 1706 for HPV82,
the amplicons corresponding to the splice junction i=4 are respectively SEQ ID NO: 1504 for HPV16, SEQ ID NO: 1518 for HPV18, SEQ ID NO: 1536 for HPV31, SEQ ID NO: 1550 for HPV33, SEQ ID NO: 1563 for HPV35, SEQ ID NO: 1576 for HPV39, SEQ ID NO: 1587 for HPV45, SEQ ID NO: 1600 for HPV51, SEQ ID NO: 1611 for HPV52, SEQ ID NO: 1627 for HPV56, SEQ ID NO: 1643 for HPV58, SEQ ID NO: 1656 for HPV59, SEQ ID NO: 1671 for HPV66, SEQ ID NO: 1684 for HPV68, SEQ ID NO: 1694 for HPV7 and SEQ ID NO: 1707 for HPV82,
the amplicons corresponding to the splice junction i=5 are respectively SEQ ID NO: 1505 for HPV16, SEQ ID NO: 1519 for HPV18, SEQ ID NO: 1537 for HPV31, SEQ ID NO: 1551 for HPV33, SEQ ID NO: 1564 for HPV35, absent for HPV39, SEQ ID NO: 1588 for HPV45, absent for HPV51, SEQ ID NO: 1612 for HPV52, SEQ ID NO: 1628 for HPV56, SEQ ID NO: 1644 for HPV58, SEQ ID NO: 1657 for HPV59, SEQ ID NO: 1672 for HPV66, absent for HPV68, absent for HPV73 and absent for HPV82,
the amplicons corresponding to the splice junction i=6 are respectively SEQ ID NO: 1506 for HPV16, SEQ ID NO: 1520 for HPV18, SEQ ID NO: 1538 for HPV31, SEQ ID NO: 1552 for HPV33, SEQ ID NO: 1565 for HPV35, absent for HPV39, absent for HPV45, SEQ ID NO: 1601 for HPV51, SEQ ID NO: 1613 for HPV52, SEQ ID NO: 1629 for HPV56, SEQ ID NO: 1645 for HPV58, absent for HPV59, absent for HPV66, SEQ ID NO: 1686 for HPV68, SEQ ID NO: 1695 for HPV73 and SEQ ID NO: 1708 for HPV82,
the amplicons corresponding to the splice junction i=7 are respectively SEQ ID NO: 1507 for HPV16, SEQ ID NO: 1521 for HPV18, SEQ ID NO: 1539 for HPV31, SEQ ID NO: 1553 for HPV33, SEQ ID NO: 1566 for HPV35, SEQ ID NO: 1578 for HPV39, SEQ ID NO: 1590 for HPV45, SEQ ID NO: 1602 for HPV51, SEQ ID NO: 1614 for HPV52, SEQ ID NO: 1630 for HPV56, SEQ ID NO: 1646 for HPV58, absent for HPV59, SEQ ID NO: 1674 for HPV66, SEQ ID NO: 1685 for HPV68, SEQ ID NO: 1696 for HPV73 and SEQ ID NO: 1709 for HPV82,
the amplicons corresponding to the splice junction i=8 are respectively SEQ ID NO: 1508 for HPV16, absent for HPV18, SEQ ID NO: 1540 for HPV31, SEQ ID NO: 1554 for HPV33, absent for HPV35, absent for HPV39, absent for HPV45, absent for HPV51, SEQ ID NO: 1615 for HPV52, SEQ ID NO: 1631 for HPV56, SEQ ID NO: 1647 for HPV58, absent for HPV59, SEQ ID NO: 1675 for HPV66, absent for HPV68, SEQ ID NO: 1697 for HPV73 and SEQ ID NO: 1710 for HPV82,
the amplicons corresponding to the splice junction i=9 are respectively SEQ ID NO: 1509 for HPV16, SEQ ID NO: 1522 for HPV18, SEQ ID NO: 1541 for HPV31, absent for HPV33, absent for HPV35, SEQ ID NO: 1579 for HPV39, SEQ ID NO: 1591 for HPV45, SEQ ID NO: 1603 for HPV51, SEQ ID NO: 1616 for HPV52, SEQ ID NO: 1632 for HPV56, absent HPV58, SEQ ID NO: 1659 for HPV59, SEQ ID NO: 1676 for HPV66, SEQ ID NO: 1687 for HPV68, SEQ ID NO: 1698 for HPV73 and SEQ ID NO: 1711 for HPV82,
the amplicons corresponding to the splice junction i=10 are respectively absent for HPV16, SEQ ID NO: 1523 for HPV18, absent for HPV31, absent for HPV33, absent for HPV35, absent for HPV39, absent for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=11 are respectively SEQ ID NO: 1510 for HPV16, SEQ ID NO: 1524 for HPV18, SEQ ID NO: 1542 for HPV31, SEQ ID NO: 1555 for HPV33, SEQ ID NO: 1567 for HPV35, SEQ ID NO: 1580 for HPV39, SEQ ID NO: 1592 for HPV45, SEQ ID NO: 1604 for HPV51, SEQ ID NO: 1617 for HPV52, SEQ ID NO: 1633 for HPV56, SEQ ID NO: 1648 for HPV58, SEQ ID NO: 1660 for HPV59, SEQ ID NO: 1677 for HPV66, SEQ ID NO: 1688 for HPV68, SEQ ID NO: 1699 for HPV73 and SEQ ID NO: 1712 for HPV82,
the amplicons corresponding to the splice junction i=12 are respectively absent for HPV16, SEQ ID NO: 1525 for HPV18, absent for HPV31, absent for HPV33, SEQ ID NO: 1568 or HPV35, absent for HPV39, absent for HPV45, absent for HPV51, SEQ ID NO: 1618 for HPV52, SEQ ID NO: 1634 for HPV56, absent for HPV58, SEQ ID NO: 1661 for HPV59, absent for HPV66, absent for HPV68, SEQ ID NO: 1700 for HPV73, absent for HPV82,
the amplicons corresponding to the splice junction i=13 are respectively absent for HPV16, SEQ ID NO: 1526 for HPV18, absent for HPV31, absent for HPV33, absent for HPV35, absent for HPV39, SEQ ID NO: 1593 for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=14 are respectively absent for HPV16, SEQ ID NO: 1527 for HPV18, absent for HPV31, absent for HPV33, absent for HPV35, absent for HPV39, absent for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=15 are respectively SEQ ID NO: 1511 for HPV16, SEQ ID NO: 1528 for HPV18, SEQ ID NO: 1543 for HPV31, SEQ ID NO: 1556 for HPV33, SEQ ID NO: 1569 for HPV35, SEQ ID NO: 1581 for HPV39, SEQ ID NO: 1594 for HPV45, SEQ ID NO: 1605 for HPV51, SEQ ID NO: 1619 for HPV52, SEQ ID NO: 1635 for HPV56, SEQ ID NO: 1649 for HPV58, SEQ ID NO: 1662 for HPV59, SEQ ID NO: 1678 for HPV66, SEQ ID NO: 1689 for HPV68, SEQ ID NO: 1701 for HPV73 and SEQ ID NO: 1713 for HPV82,
the amplicons corresponding to the splice junction i=16 are respectively SEQ ID NO: 1512 for HPV16, SEQ ID NO: 1529 for HPV18, SEQ ID NO: 1544 for HPV31, SEQ ID NO: 1557 for HPV33, SEQ ID NO: 1570 for HPV35, absent for HPV39, SEQ ID NO: 1595 for HPV45, absent for HPV51, SEQ ID NO: 1620 for HPV52, SEQ ID NO: 1636 for HPV56, SEQ ID NO: 1650 for HPV58, SEQ ID NO: 1663 for HPV59, SEQ ID NO: 1679 for HPV66, absent for HPV68, absent for HPV73 and absent for HPV82,
the amplicons corresponding to the splice junction i=17 are respectively SEQ ID NO: 1513 for HPV16, absent for HPV18, SEQ ID NO: 1545 for HPV31, SEQ ID NO: 1558 for HPV33, SEQ ID NO: 1571 for HPV35, absent for HPV39, absent for HPV45, SEQ ID NO: 1606 for HPV51, SEQ ID NO: 1621 for HPV52, SEQ ID NO: 1637 for HPV56, SEQ ID NO: 1651 for HPV58, absent for HPV59, absent for HPV66, SEQ ID NO: 1690 for HPV68, SEQ ID NO: 1702 for HPV73 and SEQ ID NO: 1714 for HPV82,
the amplicons corresponding to the splice junction i=18 are respectively SEQ ID NO:1514 for HPV16, SEQ ID NO: 1531 for HPV18, SEQ ID NO: 1546 for HPV31, SEQ ID NO: 1559 for HPV33, SEQ ID NO: 1572 for HPV35, SEQ ID NO: 1583 for HPV39, SEQ ID NO: 1597 for HPV45, SEQ ID NO: 1607 for HPV51, SEQ ID NO: 1622 for HPV52, SEQ ID NO: 1638 for HPV56, SEQ ID NO: 1652 for HPV58, SEQ ID NO: 1665 for HPV59, SEQ ID NO: 1681 for HPV66, SEQ ID NO: 1691 for HPV68, SEQ ID NO: 1703 for HPV73 and SEQ ID NO: 1715 for HPV82,
the amplicons corresponding to the splice junction i=19 are respectively absent for HPV16, SEQ ID NO: 1532 for HPV18, absent for HPV31, absent for HPV33, SEQ ID NO: 1573 for HPV35, absent for HPV39, absent for HPV45, absent for HPV51, SEQ ID NO: 1623 for HPV52, SEQ ID NO: 1639 for HPV56, absent for HPV58, SEQ ID NO: 1666 for HPV59, absent for HPV66, absent for HPV68, SEQ ID NO: 1704 for HPV73 and absent for HPV82,
the amplicons corresponding to the splice junction i=20 are respectively absent for HPV16, absent for HPV18, absent for HPV31, absent for HPV33, absent for HPV35, SEQ ID NO: 1577 for HPV39, absent for HPV45, absent for HPV51, absent for HPV52, absent for HPV56, absent for HPV58, SEQ ID NO: 1658 for HPV59, absent for HPV66, absent for HPV68, absent for HPV73 and absent for HPV82,
the amplicons corresponding to the splice junction i=21 are respectively absent for HPV16, absent for HPV18, absent for HPV31, absent for HPV33, absent for HPV35, SEQ ID NO: 1582 for HPV39, absent for HPV45, absent for HPV51, absent for HPV52, absent for HPV56, absent for HPV58, SEQ ID NO: 1664 for HPV59, absent for HPV66, absent for HPV68, absent for HPV73 and absent for HPV82,
the amplicons corresponding to the splice junction i=22 are respectively absent for HPV16, absent for HPV18, absent for HPV31, absent for HPV33, absent for HPV35, SEQ ID NO: 1575 for HPV39, absent for HPV45, absent for HPV51, absent for HPV52, absent for HPV56, absent for HPV58, SEQ ID NO: 1655 for HPV59, absent for HPV66, absent for HPV68, absent for HPV73 and absent for HPV82,
the amplicons corresponding to the splice junction i=23 are respectively absent for HPV16, absent for HPV18, absent for HPV31, absent for HPV33, absent for HPV35, absent for HPV39, SEQ ID NO: 1586 for HPV45, absent for HPV51, absent for HPV52, absent for HPV56, absent for HPV58, absent for HPV59, SEQ ID NO: 1670 for HPV66, absent for HPV68, absent for HPV73 and absent for HPV82,
the amplicons corresponding to the splice junction i=24 are respectively absent for HPV16, absent for HPV18, absent for HPV31, absent for HPV33, absent for HPV35, absent for HPV39, SEQ ID NO: 1589 for HPV45, absent for HPV51, absent for HPV52, absent for HPV56, absent for HPV58, absent for HPV59, SEQ ID NO: 1673 for HPV66, absent for HPV68, absent for HPV73 and absent for HPV82,
the amplicons corresponding to the splice junction i=25 are respectively absent for HPV16, SEQ ID NO: 1530 for HPV18, absent for HPV31, absent for HPV33, absent for HPV35, absent for HPV39, SEQ ID NO: 1596 HPV45, absent for HPV51, absent for HPV52, absent for HPV56, absent for HPV58, absent for HPV59, SEQ ID NO: 1680 for HPV66, absent for HPV68, absent for HPV73 and absent for HPV82.

The in vitro method for detecting HSIL may comprise a step of treatment of the biological sample with a solution comprising 30-60 wt % of methanol and 40-70 wt % of water such as preservCyt.

Composition of Primers for HPV Typing

The present invention relates to a composition of primers for typing HPV selected from the group consisting of:
at least one pair of primers of each of the first to the thirteenth and optionally of the fourteenth and/or fifteenth and/or sixteenth subsets of the splice junctions set of pairs of primers as defined above,
a second set of primers, called unsplice junctions set of primers,
a third set of primers, called genomic set of primers, and
a fourth set of primers, called fusion set of primers.

The present invention relates to a composition of primers for typing HPV comprising the set of primers selected from the group consisting of the splice junctions set of primers as defined above, a second set of primers, called unsplice junctions set of primers, a third set of primers, called genomic set of primers, and a fourth set of primers, called fusion set of primers.

The present invention also relates to a composition of primers for typing HPV comprising the splice junctions set of primers as defined above and an additional set of primers selected from the group consisting of a second set of primers, called unsplice junctions set of primers, a third set of primers, called genomic set of primers and a fourth set of primers, called fusion set of primers.

The method and composition of primers for typing HPV according the invention provides results as good as current gold standard test for HPV typing.

Moreover, the method and the composition of primers of the invention replace the current combination of cytology (Pap smear) and HPV molecular screening by a single molecular test for both the detection of high-risk or putative high-risk HPV and the triage of women at risk of transforming infection, before colposcopy. In particular, the splice junctions set of primers may be used for both detecting a HSIL lesion and typing HPV.

Preferably, the composition of primers for typing HPV of the invention comprises the splice junctions set of primers as defined above, an unsplice junctions set of primers, a genomic set of primers and a fusion set of primers.

Each of the unspliced junctions set, the genomic set of primers and the fusion set of primers may comprise a subset of pairs of primers specific of each high risk HPV and optionally a subset of primers specific of each putative high risk HPV.

The composition of primers for typing HPV of the invention may also comprises an additional fifth set of primers, called human set of primers. The human set of primers is as defined above.

The primers of unsplice junctions set of primers target high risk and optionally of putative high risk HPV genomic regions spanning either splice donor or splice acceptor sites in the absence of any splice event.

The unsplice junctions set of primers may comprise:
at least 1, at least 2, at least 3, at least 4, at least 5, at least 6, at least 7, at least 8, at least 9, at least 10 or at least 11, or 1, 2, 3, 4, 5, 6, 7, 8, 9, 10 or 11 pairs of primers of a first subset of HPV16 specific pairs of primers having the nucleic acid sequence selected from the group consisting of SEQ ID NO: 431-432, to SEQ ID NO: 451-452;
at least 1, at least 2, at least 3, at least 4, at least 5, at least 6, at least 7, at least 8, at least 9, at least 10, at least 11 or at least 12, or 1, 2, 3, 4, 5, 6, 7, 8, 9, 10, 11 or 12 pairs of primers of a second subset of HPV18 specific pairs of primers having the nucleic acid sequence selected from the group consisting of SEQ ID NO: 453-454 to SEQ ID NO: 475-476;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a third subset of HPV31 specific pairs of primers having the nucleic acid sequence selected from the group consisting of SEQ ID NO: 477-478 to SEQ ID NO: 497-498;
at least 1, at least 2, at least 3, at least 4, at least 5, at least 6, at least 7, at least 8 or at least 9, or 1, 2, 3, 4, 5, 6, 7, 8 or 9 pairs of primers of a fourth subset of HPV33 specific pairs of primers having the nucleic acid sequence selected from the group consisting of SEQ ID NO: 499-500 to SEQ ID NO: 515-516;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a fifth subset of HPV35 specific pairs of primers having the nucleic acid sequence selected from the group consisting of SEQ ID NO: 517-518 to SEQ ID NO: 535-536;
at least 1, at least 2, at least 3, at least 4, at least 5, at least 6, at least 7 or at least 8, or 1, 2, 3, 4, 5, 6, 7 or 8 pairs of primers of a sixth subset of HPV39 specific pairs of primers having the nucleic acid sequence selected from the group consisting of SEQ ID NO: 537-538 to SEQ ID NO: 551-552;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a seventh subset of HPV45 specific pairs of primers having the nucleic acid sequence selected from the group consisting of SEQ ID NO: 553-554 to SEQ ID NO: 571-572;
at least 1, at least 2, at least 3, at least 4, at least 5, at least 6, at least 7, at least 8 or at least 9, or 1, 2, 3, 4, 5, 6, 7, 8 or 9 pairs of primers of a eighth subset of HPV51 specific pairs of primers having the nucleic acid sequence selected from the group consisting of SEQ ID NO: 573-574 to SEQ ID NO: 589-590;
at least 1, at least 2, at least 3, at least 4, at least 5, at least 6, at least 7, at least 8, at least 9, at least 10 or at least 11, or 1, 2, 3, 4, 5, 6, 7, 8, 9, 10 or 11 pairs of primers of a ninth subset of HPV52 specific pairs of primers having the nucleic acid sequence selected from the group consisting of SEQ ID NO: 591-592 to SEQ ID NO: 611-612;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a tenth subset of HPV56 specific pairs of primers having the nucleic acid sequence selected from the group consisting of SEQ ID NO: 613-614 to SEQ ID NO: 631-632;
at least 1, at least 2, at least 3, at least 4, at least 5, at least 6, at least 7 or at least 8, or 1, 2, 3, 4, 5, 6, 7 or 8 pairs of primers of an eleventh subset of HPV58 specific pairs of primers having the nucleic acid sequence selected from the group consisting of SEQ ID NO: 633-634 to SEQ ID NO: 647-648;
at least 1, at least 2, at least 3, at least 4, at least 5, at least 6, at least 7 or at least 8, or 1, 2, 3, 4, 5, 6, 7 or 8 pairs of primers of a twelfth subset of HPV59 specific pairs of primers having the nucleic acid sequence selected from the group consisting of SEQ ID NO: 649-650 to SEQ ID NO: 663-664;
at least 1, at least 2, at least 3, at least 4, at least 5, at least 6, at least 7 or at least 8, or 1, 2, 3, 4, 5, 6, 7 or 8 pairs of primers of a thirteenth subset of HPV66 specific pairs of primers having the nucleic acid sequence selected from the group consisting of SEQ ID NO: 665-666 to SEQ ID NO: 679-680.

The unsplice junctions set of primers may further comprise:
at least 1, at least 2, at least 3, at least 4, at least 5, at least 6, at least 7, at least 8 or at least 9, or 1, 2, 3, 4, 5, 6, 7, 8 or 9 pairs of primers of a fourteenth subset of HPV68 specific pairs of primers having the nucleic acid sequence selected from the group consisting of SEQ ID NO: 681-682 to SEQ ID NO: 697-698; and/or
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of the fifteenth subset of HPV73 specific pairs of primers having the nucleic acid sequence selected from the group consisting of SEQ ID NO: 699-700 to SEQ ID NO: 717-718; and/or
at least 1, at least 2, at least 3, at least 4, at least 5, at least 6, at least 7, at least 8 or at least 9, or 1, 2, 3, 4, 5, 6, 7, 8 or 9 pairs of primers of the sixteenth subset of HPV82 specific pairs of primers having the nucleic acid sequence selected from the group consisting of SEQ ID NO: 719-720 to SEQ ID NO: 735-736.

In one embodiment, the unsplice junctions set of primers comprises the pairs of primers having the nucleic acid sequence SEQ ID NO: 431-432 to SEQ ID NO: 679-680 and optionally of SEQ ID NO: 681-682 to SEQ ID NO: 697-698 and/or SEQ ID NO: 699-700 to SEQ ID NO: 717-718 and/or SEQ ID NO: 719-720 to SEQ ID NO: 735-736.

In a preferred embodiment, the unsplice junctions set of primers comprises the pairs of primers having the nucleic acid sequence SEQ ID NO: 431-432 to SEQ ID NO: 735-736.

In one embodiment, the unsplice junctions set of primers consists of the pairs of primers having the nucleic acid sequence SEQ ID NO: 431-432 to SEQ ID NO: 679-680 and optionally of SEQ ID NO: 681-682 to SEQ ID NO: 697-698 and/or SEQ ID NO: 699-700 to SEQ ID NO: 717-718 and/or SEQ ID NO: 719-720 to SEQ ID NO: 735-736.

In a preferred embodiment, the unsplice junctions set of primers consists of the pairs of primers having the nucleic acid sequence SEQ ID NO: 431-432 to SEQ ID NO: 735-736.

The unsplice junctions set of primers may comprise:
at least 1, at least 2, at least 3, at least 4, at least 5, at least 6, at least 7, at least 8, at least 9, at least 10 or at least 11, or 1, 2, 3, 4, 5, 6, 7, 8, 9, 10 or 11 pairs of primers a first subset of HPV16 specific pairs of primers able to produce the amplicons having the nucleic acid sequence selected from the group consisting of SEQ ID NO: 1716 to SEQ ID NO: 1726;
at least 1, at least 2, at least 3, at least 4, at least 5, at least 6, at least 7, at least 8, at least 9, at least 10, at least 11 or at least 12, or 1, 2, 3, 4, 5, 6, 7, 8, 9, 10, 11 or 12 pairs of primers of a second subset of HPV18 specific pairs of primers able to produce the amplicons having the nucleic acid sequence selected from the group consisting of SEQ ID NO: 1727 to SEQ ID NO: 1738;
at least 1, at least 2, at least 3, at least 4, at least 5, at least 6, at least 7, at least 8, at least 9, at least 10, at least 11 or at least 12, or 1, 2, 3, 4, 5, 6, 7, 8, 9, 10, 11 or 12 pairs of primers of a third subset of HPV31 specific pairs of primers able to produce the amplicons having the nucleic acid sequence selected from the group consisting of SEQ ID NO: 1739 to SEQ ID NO: 1749;
at least 1, at least 2, at least 3, at least 4, at least 5, at least 6, at least 7, at least 8 or at least 9, or 1, 2, 3, 4, 5, 6, 7, 8 or 9 pairs of primers of a fourth subset of HPV33 specific pairs of primers able to produce the amplicons having the nucleic acid sequence selected from the group consisting of SEQ ID NO: 1750 to SEQ ID NO: 1758;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a fifth subset of HPV35 specific pairs of primers able to produce the amplicons having the nucleic acid sequence selected from the group consisting of SEQ ID NO: 1759 to SEQ ID NO: 1768;
at least 1, at least 2, at least 3, at least 4, at least 5, at least 6, at least 7 or at least 8, or 1, 2, 3, 4, 5, 6, 7 or 8 pairs of primers of a sixth subset of HPV39 specific pairs of primers able to produce the amplicons having the nucleic acid sequence selected from the group consisting of SEQ ID NO: 1769 to SEQ ID NO: 1776;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a seventh subset of HPV45 specific pairs of primers able to produce the amplicons having the nucleic acid sequence selected from the group consisting of SEQ ID NO: 1777 to SEQ ID NO: 1786;
at least 1, at least 2, at least 3, at least 4, at least 5, at least 6, at least 7, at least 8 or at least 9, or 1, 2, 3, 4, 5, 6, 7, 8 or 9 pairs of primers of a eighth subset of HPV51 specific pairs of primers able to produce the amplicons having the nucleic acid sequence selected from the group consisting of SEQ ID NO: 1787 to SEQ ID NO: 1795;
at least 1, at least 2, at least 3, at least 4, at least 5, at least 6, at least 7, at least 8, at least 9, at least 10 or at least 11, or 1, 2, 3, 4, 5, 6, 7, 8, 9, 10 or 11 pairs of primers of a ninth subset of HPV52 specific pairs of primers able to produce the amplicons having the nucleic acid sequence selected from the group consisting of SEQ ID NO: 1796 to SEQ ID NO: 1806;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a tenth subset of HPV56 specific pairs of primers able to produce the amplicons having the nucleic acid sequence selected from the group consisting of SEQ ID NO: 1807 to SEQ ID NO: 1816;
at least 1, at least 2, at least 3, at least 4, at least 5, at least 6, at least 7 or at least 8, or 1, 2, 3, 4, 5, 6, 7 or 8 pairs of primers of an eleventh subset of HPV58 specific pairs of primers able to produce the amplicons having the nucleic acid sequence selected from the group consisting of SEQ ID NO: 1817 to SEQ ID NO: 1824;
at least 1, at least 2, at least 3, at least 4, at least 5, at least 6, at least 7 or at least 8, or 1, 2, 3, 4, 5, 6, 7 or 8 pairs of primers of a twelfth subset of HPV59 specific pairs of primers able to produce the amplicons having the nucleic acid sequence selected from the group consisting of SEQ ID NO: 1825 to SEQ ID NO: 1832;
at least 1, at least 2, at least 3, at least 4, at least 5, at least 6 or at least 7, or 1, 2, 3, 4, 5, 6 or 7 pairs of primers of a thirteenth subset of HPV66 specific pairs of primers able to produce the amplicons having the nucleic acid sequence selected from the group consisting of SEQ ID NO: 1833 to SEQ ID NO: 1840.

The unsplice junctions set of primers may further comprise:
at least 1, at least 2, at least 3, at least 4, at least 5, at least 6, at least 7, at least 8 or at least 9, or 1, 2, 3, 4, 5, 6, 7, 8 or 9 pairs of primers of a fourteenth subset of HPV68 specific pairs of primers able to produce the amplicons having the nucleic acid sequence selected from the group consisting of SEQ ID NO: 1841 to SEQ ID NO: 1849;
at least 1, at least 2, at least 3, at least 4, at least 5, at least 6, at least 7, at least 8, at least 9 or at least 10, or 1, 2, 3, 4, 5, 6, 7, 8, 9 or 10 pairs of primers of a fifteenth subset of HPV73 specific pairs of primers able to produce the amplicons having the nucleic acid sequence selected from the group consisting of SEQ ID NO: 1850 to SEQ ID NO: 1859,
at least 1, at least 2, at least 3, at least 4, at least 5, at least 6, at least 7, at least 8 or at least 9, or 1, 2, 3, 4, 5, 6, 7, 8 or 9 pairs of primers of a sixteenth subset of HPV82 specific pairs of primers able to produce the amplicons having the nucleic acid sequence selected from the group consisting of SEQ ID NO: 1860 to SEQ ID NO: 1868.

In one embodiment, the unsplice junctions set of primers comprises the pairs of primers able to produce the amplicons having the nucleic acid sequence SEQ ID NO: 1716 to SEQ ID NO: 1840 and optionally of SEQ ID NO: 1841 to SEQ ID NO: 1849 and/or SEQ ID NO: 1850 to SEQ ID NO: 1859 and/or SEQ ID NO: 1860 to SEQ ID NO: 1868.

In a preferred embodiment, the unsplice junctions set of primers comprises the pairs of primers able to produce the amplicons having the nucleic acid sequence SEQ ID NO: 1716 to SEQ ID NO: 1726 to SEQ ID NO: 1860 to SEQ ID NO: 1868.

In one embodiment, the unsplice junctions set of primers consists of the pairs of primers able to produce the amplicons having the nucleic acid sequence SEQ ID NO: 1716 to SEQ ID NO: 1840 and optionally of SEQ ID NO: 1841 to SEQ ID NO: 1849 and/or SEQ ID NO: 1850 to SEQ ID NO: 1859 and/or SEQ ID NO: 1860 to SEQ ID NO: 1868.

In a preferred embodiment, the unsplice junctions set of primers consists of the pairs of primers having the nucleic acid sequence SEQ ID NO: 1716 to SEQ ID NO: 1726 to SEQ ID NO: 1868.

The primers of the genomic set of primers target high risk and optionally of putative high risk HPV genomic regions away from any splice donor or splice acceptor sites,

The genomic set of primers may comprise:
at least 1, at least 2, at least 3 or at least 4, or 1, 2, 3 or 4 pairs of primers of a first subset of HPV16 specific pairs of primers having the nucleic acid sequence selected from the group consisting of SEQ ID NO: 737-738, SEQ ID NO: 739-740, SEQ ID NO: 741-742 and SEQ ID NO: 743-744;
at least 1, at least 2, at least 3 or at least 4 or 1, 2, 3 or 4 pairs of primers of a second subset of HPV18 specific pairs of primers having the nucleic acid sequence selected from the group consisting of SEQ ID NO: 745-746, SEQ ID NO: 747-748, SEQ ID NO: 749-750 and SEQ ID NO: 751-752;
at least 1, at least 2, at least 3 or at least 4, or 1, 2, 3 or 4 pairs of primers of a third subset of HPV31 specific pairs of primers having the nucleic acid sequence selected from the group consisting of SEQ ID NO: 753-754, SEQ ID NO: 755-756, SEQ ID NO: 757-758 and SEQ ID NO: 759-760;
at least 1, at least 2, at least 3 or at least 4, or 1, 2, 3 or 4 pairs of primers of a fourth subset HPV33 of specific pairs of primers having the nucleic acid sequence selected from the group consisting of SEQ ID NO: 761-762, SEQ ID NO: 763-764, SEQ ID NO: 765-766 and SEQ ID NO: 767-768;
at least 1, at least 2, at least 3 or at least 4, or 1, 2, 3 or 4 pairs of primers of a fifth subset of HPV35 specific pairs of primers having the nucleic acid sequence selected from the group consisting of SEQ ID NO: 769-770, SEQ ID NO: 771-772, SEQ ID NO: 773-774 and SEQ ID NO: 775-776;
at least 1, at least 2, at least 3 or at least 4, or 1, 2, 3 or 4 pairs of primers of a sixth subset of HPV39 specific pairs of primers having the nucleic acid sequence selected from the group consisting of SEQ ID NO: 777-778, SEQ ID NO: 779-780, SEQ ID NO: 781-782 and SEQ ID NO: 783-784;
at least 1, at least 2, at least 3 or at least 4, or 1, 2, 3 or 4 pairs of primers of a seventh subset of HPV45 specific pairs of primers having the nucleic acid sequence selected from the group consisting of SEQ ID NO: 785-786, SEQ ID NO: 787-788, SEQ ID NO: 789-790 and SEQ ID NO: 791-792;
at least 1, at least 2, at least 3 or at least 4, or 1, 2, 3 or 4 pairs of primers of a eighth subset of HPV51 specific pairs of primers having the nucleic acid sequence selected from the group consisting of SEQ ID NO: 793-794, SEQ ID NO: 795-796, SEQ ID NO: 797-798 and SEQ ID NO: 799-800;
at least 1, at least 2, at least 3 or at least 4, or 1, 2, 3 or 4 pairs of primers of a ninth subset of HPV52 specific pairs of primers having the nucleic acid sequence selected from the group consisting of SEQ ID NO: 801-802, SEQ ID NO: 803-804, SEQ ID NO: 805-806 and SEQ ID NO: 807-808;
at least 1, at least 2, at least 3 or at least 4, or 1, 2, 3 or 4 pairs of primers a tenth subset of HPV56 specific pairs of primers having the nucleic acid sequence selected from the group consisting of SEQ ID NO: 809-810, SEQ ID NO: 811-812, SEQ ID NO: 813-814 and SEQ ID NO: 815-816;
at least 1, at least 2, at least 3 or at least 4, or 1, 2, 3 or 4 pairs of primers of an eleventh subset of HPV58 specific pairs of primers having the nucleic acid sequence selected from the group consisting of SEQ ID NO: 817-818, SEQ ID NO: 819-820, SEQ ID NO: 821-822 and SEQ ID NO: 823-824;
at least 1, at least 2, at least 3 or at least 4, or 1, 2, 3 or 4 pairs of primers of a twelfth subset of HPV59 specific pairs of primers having the nucleic acid sequence selected from the group consisting of SEQ ID NO: 825-826, SEQ ID NO: 827-828, SEQ ID NO: 829-830 and SEQ ID NO: 831-832;
at least 1, at least 2, at least 3 or at least 4, or 1, 2, 3 or 4 pairs of primers of a thirteenth subset of HPV66 specific pairs of primers having the nucleic acid sequence selected from the group consisting of SEQ ID NO: 833-834, SEQ ID NO: 835-836, SEQ ID NO: 837-838 and SEQ ID NO: 839-840.

The genomic set of primers may further comprise:
at least 1, at least 2, at least 3 or at least 4, or 1, 2, 3 or 4 pairs of primers of a fourteenth subset of HPV68 specific pairs of primers having the nucleic acid sequence selected from the group consisting of SEQ ID NO: 841-842, SEQ ID NO: 843-844, SEQ ID NO: 845-846 and SEQ ID NO: 847-848;
at least 1, at least 2, at least 3 or at least 4, or 1, 2, 3 or 4 pairs of primers of a fifteenth subset of HPV73 specific pairs of primers having the nucleic acid sequence selected from the group consisting of SEQ ID NO: 849-850, SEQ ID NO: 851-852, SEQ ID NO: 853-854 and SEQ ID NO: 855-856;
at least 1, at least 2, at least 3 or at least 4, or 1, 2, 3 or 4 pairs of primers of a sixteenth subset of HPV82 specific pairs of primers having the nucleic acid sequence selected from the group consisting of SEQ ID NO: 857-858, SEQ ID NO: 859-860, SEQ ID NO: 861-862 and SEQ ID NO: 863-864.

In one embodiment, the genomic set of primers comprises the pairs of primers having the nucleic acid sequence SEQ ID NO: 737-738 to SEQ ID NO: 839-840 and optionally of SEQ ID NO: 841-842 to SEQ ID NO: 847-848 and/or SEQ ID NO: 849-850 to SEQ ID NO: 853-854 and SEQ ID NO: 855-856 and/or SEQ ID NO: 857-858 to SEQ ID NO: 863-864. In a preferred embodiment, the genomic set of primers comprises the pairs of primers having the nucleic acid sequence SE SEQ ID NO: 737-738 to SEQ ID NO: 863-864.

In one embodiment, the genomic set of primers consists of the pairs of primers having the nucleic acid sequence SEQ ID NO: 737-738 to SEQ ID NO: 839-840 and optionally of SEQ ID NO: 841-842 to SEQ ID NO: 847-848 and/or SEQ ID NO: 849-850 to SEQ ID NO: 853-854 and SEQ ID NO: 855-856 and/or SEQ ID NO: 857-858 to SEQ ID NO: 863-864. In a preferred embodiment, the genomic set of primers consists of the pairs of primers having the nucleic acid sequence SEQ ID NO: 737-738 to SEQ ID NO: 863-864.

The genomic set of primers may comprise:
at least 1, at least 2, at least 3 or at least 4, or 1, 2, 3 or 4 pairs of primers of first subset of HPV16 specific pairs of primers able to produce the amplicons having the nucleic acid sequence selected from the group consisting of SEQ ID NO: 1869 to SEQ ID NO: 1872;
at least 1, at least 2, at least 3 or at least 4, or 1,2, 3 or 4 pairs of primers the second subset of HPV18 specific pairs of primers able to produce the amplicons having the nucleic acid sequence selected from the group consisting of SEQ ID NO: 1873 to SEQ ID NO: 1876;
at least 1, at least 2, at least 3 or at least 4, or 1, 2, 3 or 4 pairs of primers of a third subset of HPV31 specific pairs of primers able to produce the amplicons having the nucleic acid sequence selected from the group consisting of SEQ ID NO: 1877 to SEQ ID NO: 1880;
at least 1, at least 2, at least 3 or at least 4, or 1, 2, 3 or 4 pairs of primers of a fourth subset of HPV33 specific pairs of primers able to produce the amplicons having the nucleic acid sequence selected from the group consisting of SEQ ID NO: 1881 to SEQ ID NO: 1884;
at least 1, at least 2, at least 3 or at least 4, or 1, 2, 3 or 4 pairs of primers of a fifth subset of HPV35 specific pairs of primers able to produce the amplicons having the nucleic acid sequence selected from the group consisting of SEQ ID NO: 1885 to SEQ ID NO: 1888;
at least 1, at least 2, at least 3 or at least 4, or 1, 2, 3 or 4 pairs of primers of a sixth subset of HPV39 specific pairs of primers able to produce the amplicons having the nucleic acid sequence selected from the group consisting of SEQ ID NO: 1889 to SEQ ID NO: 1892;
at least 1, at least 2, at least 3 or at least 4, or 1, 2, 3 or 4 pairs of primers of a seventh subset of HPV45 specific pairs of primers able to produce the amplicons having the nucleic acid sequence selected from the group consisting of SEQ ID NO: 1893 to SEQ ID NO: 1896;
at least 1, at least 2, at least 3 or at least 4, or 1, 2, 3 or 4 pairs of primers of a eighth subset of HPV51 specific pairs of primers able to produce the amplicons having the nucleic acid sequence selected from the group consisting of SEQ ID NO: 1897 to SEQ ID NO: 1900;
at least 1, at least 2, at least 3 or at least 4, or 1, 2, 3 or 4 pairs of primers of a ninth subset of HPV52 specific pairs of primers able to produce the amplicons having the nucleic acid sequence selected from the group consisting of SEQ ID NO: 1901 to SEQ ID NO: 1904;
at least 1, at least 2, at least 3 or at least 4, or 1, 2, 3 or 4 pairs of primers of a tenth subset of HPV56 specific pairs of primers able to produce the amplicons having the nucleic acid sequence selected from the group consisting of SEQ ID NO: 1905 to SEQ ID NO: 1908;
at least 1, at least 2, at least 3 or at least 4, or 1, 2, 3 or 4 pairs of primers of an eleventh subset of HPV58 specific pairs of primers specific able to produce the amplicons having the nucleic acid sequence selected from the group consisting of SEQ ID NO: 1909 to SEQ ID NO: 1912;
at least 1, at least 2, at least 3 or at least 4, or 1, 2, 3 or 4 pairs of primers of a twelfth subset of HPV59 specific pairs of primers able to produce the amplicons having the nucleic acid sequence selected from the group consisting of SEQ ID NO: 1913 to SEQ ID NO: 1916;
at least 1, at least 2, at least 3 or at least 4, or 1, 2, 3 or 4 pairs of primers of a thirteenth subset of HPV66 specific pairs of primers able to produce the amplicons having the nucleic acid sequence selected from the group consisting of SEQ ID NO: 1917 to SEQ ID NO: 1920.

The genomic set of primers may further comprise:
at least 1, at least 2, at least 3 or at least 4, or 1, 2, 3 or 4 pairs of primers of a fourteenth subset of HPV68 specific pairs of primers able to produce the amplicons having the nucleic acid sequence selected from the group consisting of SEQ ID NO: 1921 to SEQ ID NO: 1924; and/or
at least 1, at least 2, at least 3 or at least 4, or 1, 2, 3 or 4 pairs of primers of a fifteenth subset of HPV73 specific pairs of primers able to produce the amplicons having the nucleic acid sequence selected from the group consisting of SEQ ID NO: 1925 to SEQ ID NO: 1928; and/or
at least 1, at least 2, at least 3 or at least 4, or 1, 2, 3 or 4 pairs of primers of a sixteenth subset of HPV82 specific pairs of primers able to produce the amplicons having the nucleic acid sequence selected from the group consisting of SEQ ID NO: 1929 to SEQ ID NO: 1932.

In one embodiment, the genomic set of primers comprises the pairs of primers able to produce the amplicons having the nucleic acid sequence SEQ ID NO: 1869 to SEQ ID NO: 1920 and optionally of SEQ ID NO: 1921 to SEQ ID NO: 1924 and/or SEQ ID NO: 1925 to SEQ ID NO: 1928 and/or SEQ ID NO: 1929 to SEQ ID NO: 1932.

In a preferred embodiment, the genomic set of primers comprises the pairs of primers able to produce the amplicons having the nucleic acid sequence SEQ ID NO: 1869 to SEQ ID NO: 1932.

In one embodiment, the genomic set of primers consists of the pairs of primers able to produce the amplicons having the nucleic acid sequence SEQ ID NO: 1869 to SEQ ID NO: 1833 to 1840 and optionally of SEQ ID NO: 1921 to SEQ ID NO: 1924 and/or SEQ ID NO: 1925 to SEQ ID NO: 1928 and/or SEQ ID NO: 1929 to SEQ ID NO: 1932.

In a preferred embodiment, the genomic set of primers consists of the pairs of primers having the nucleic acid sequence SEQ ID NO: 1869 to SEQ ID NO: 1932.

The primers of fusion set of primers target high risk and optionally of putative high risk HPV fusion transcripts.

The primers of fusion set of primers may comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a first subset of HPV16 specific pairs of primers having the nucleic acid sequence selected from the group consisting of SEQ ID NO: 865-866 to SEQ ID NO: 899-900;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a second subset of HPV18 specific pairs of primers having the nucleic acid sequence selected from the group consisting of SEQ ID NO: 901-902 to SEQ ID NO: 935-936;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a third subset of HPV31 specific pairs of primers having the nucleic acid sequence selected from the group consisting of SEQ ID NO: 937-938 to SEQ ID NO: 971-972;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourth subset of HPV33 specific pairs of primers having the nucleic acid sequence selected from the group consisting of SEQ ID NO: 973-974 to SEQ ID NO: 1007-1008;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifth subset of HPV35 specific pairs of primers having the nucleic acid sequence selected from the group consisting of SEQ ID NO: 1009-1010 to SEQ ID NO: 1043-1044;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixth subset of HPV39 specific pairs of primers having the nucleic acid sequence selected from the group consisting of SEQ ID NO: 1045-1046 to SEQ ID NO: 1079-1080;
at least 1, at least 2, at least 3, at least 4, at least 5, at least 6, at least 7, at least 8, at least 9, at least 10, at least 11, at least 12, at least 13, at least 14, at least 15, at least 16, preferably at least 17, more preferably 18 or 1, 2, 3, 4, 5, 6, 7, 8, 9, 10, 11, 12, 13, 14, 15, 16, 17 or 18 pairs of primers of a seventh subset of HPV45 specific pairs of primers having the nucleic acid sequence selected from the group consisting of SEQ ID NO: 1081-1082 to SEQ ID NO: 1115-1116.
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a eighth subset of HPV51 specific pairs of primers having the nucleic acid sequence selected from the group consisting of SEQ ID NO: 1117-1118 to SEQ ID NO: 1151-1152;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a ninth subset of HPV52 specific pairs of primers having the nucleic acid sequence selected from the group consisting of SEQ ID NO: 1153-1154 to SEQ ID NO: 1187-1188;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a tenth subset of HPV56 specific pairs of primers having the nucleic acid sequence selected from the group consisting of SEQ ID NO: 1189-1190 to SEQ ID NO: 1223-1224;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of an eleventh subset of HPV58 specific pairs of primers having the nucleic acid sequence selected from the group consisting of SEQ ID NO: 1225-1226 to SEQ ID NO: 1259-1260;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a twelfth subset of HPV59 specific pairs of primers having the nucleic acid sequence selected from the group consisting of SEQ ID NO: 1261-1262 to SEQ ID NO: 1295-1296;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a thirteenth subset of HPV66 specific pairs of primers having the nucleic acid sequence selected from the group consisting of SEQ ID NO: 1297-1298 to SEQ ID NO: 1331-1332.

The fusion set of primers may further comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourteenth subset of HPV68 specific pairs of primers having the nucleic acid sequence selected from the group consisting of SEQ ID NO: 1333-1334 to SEQ ID NO: 1367-1368;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifteenth subset of HPV73 specific pairs of primers having the nucleic acid sequence selected from the group consisting of SEQ ID NO:1369-1370 to SEQ ID NO: 1403-1404;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixteenth subset of HPV82 specific pairs of primers having the nucleic acid sequence selected from the group consisting of SEQ ID NO: 1405-1406 to SEQ ID NO: 1439-1440.

In one embodiment, the fusion set of primers comprises the pairs of primers having the nucleic acid sequence SEQ ID NO: 865-866 to SEQ ID NO: 1331-1332 and optionally of SEQ ID NO: 1333-1334 to SEQ ID NO: 1367-1368 and/or SEQ ID NO: 1369-1370 to SEQ ID NO: 1403-1404 and/or SEQ ID NO: 1405-1406 to SEQ ID NO: 1439-1440.

In a preferred embodiment, the fusion set of primers comprises the pairs of primers having the nucleic acid sequence SEQ ID NO: 865-866 to SEQ ID NO: 1439-1440.

In one embodiment, the fusion set of primers consists of the pairs of primers having the nucleic acid sequence SEQ ID NO: 865-866 to SEQ ID NO: 1331-1332 and optionally of SEQ ID NO: 1333-1334 to SEQ ID NO: 1367-1368 and/or SEQ ID NO: 1369-1370 to SEQ ID NO: 1403-1404 and/or SEQ ID NO: 1405-1406 to SEQ ID NO: 1439-1440.

In a preferred embodiment, the fusion set of primers consists of the pairs of primers having the nucleic acid sequence SEQ ID NO: 865-866 to SEQ ID NO: 1439-1440.

The primers of fusion set of primers may comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a first subset of HPV16 specific pairs of primers able to produce the amplicons having the nucleic acid sequence selected from the group consisting of SEQ ID NO: 1933 to 1 SEQ ID NO: 1950;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a second subset of HPV18 specific pairs of primers able to produce the amplicons having the nucleic acid sequence selected from the group consisting of SEQ ID NO: 1951 to SEQ ID NO: 1968;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a third subset of HPV31 specific pairs of primers able to produce the amplicons having the nucleic acid sequence selected from the group consisting of SEQ ID NO: 1969 to SEQ ID NO: 1986;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourth subset of HPV33 specific pairs of primers able to produce the amplicons having the nucleic acid sequence selected from the group consisting of SEQ ID NO: 1987 to SEQ ID NO: 2004;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifth subset of HPV35 specific pairs of primers able to produce the amplicons having the nucleic acid sequence selected from the group consisting of SEQ ID NO: 2005 to SEQ ID NO: 2022;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixth subset of HPV39 specific pairs of primers able to produce the amplicons having the nucleic acid sequence selected from the group consisting of SEQ ID NO: 2023 to SEQ ID NO: 2040;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a seventh subset of HPV45 specific pairs of primers selected from the group consisting of SEQ ID NO: 2041 to SEQ ID NO: 2058,
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a eighth subset of HPV51 specific pairs of primers able to produce the amplicons having the nucleic acid sequence selected from the group consisting of SEQ ID NO: 2059 to SEQ ID NO: 2076;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a ninth subset of HPV52 specific pairs of primers able to produce the amplicons having the nucleic acid sequence selected from the group consisting of SEQ ID NO: 2077 to SEQ ID NO: 2094;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a tenth subset of HPV56 specific pairs of primers able to produce the amplicons having the nucleic acid sequence selected from the group consisting of SEQ ID NO: 2095 to SEQ ID NO: 2112;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of an eleventh subset of HPV58 specific pairs of primers able to produce the amplicons having the nucleic acid sequence selected from the group consisting of SEQ ID NO: 2113 to SEQ ID NO: 2130;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a twelfth subset of HPV59 specific pairs of primers able to produce the amplicons having the nucleic acid sequence selected from the group consisting of SEQ ID NO: 2131 to SEQ ID NO: 2148;
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a thirteenth subset of HPV66 specific pairs of primers able to produce the amplicons having the nucleic acid sequence selected from the group consisting of SEQ ID NO: 2149 to SEQ ID NO: 2166.

The fusion set may also comprise:
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fourteenth subset of HPV68 specific pairs of primers able to produce the amplicons having the nucleic acid sequence selected from the group consisting of SEQ ID NO: 2167 to SEQ ID NO: 2184; and/or
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a fifteenth subset of HPV73 specific pairs of primers able to produce the amplicons having the nucleic acid sequence selected from the group consisting of SEQ ID NO:2185 to SEQ ID NO: 2202;and/or
at least 1, at least 2, at least 3, at least 4, at least 5, at least 6, at least 7, at least 8, at least 9, at least 10, at least 11, at least 12, at least 13, at least 14, at least 15, at least 16, at least 17 or at least 18, or 1, 2, 3, 4, 5, 6, 7, 8, 9, 10, 11, 12, 13, 14, 15, 16, 17 or 18 pairs of primers of a sixteenth subset of HPV82 specific pairs of primers able to produce the amplicons having the nucleic acid sequence selected from the group consisting of SEQ ID NO: 2203 to SEQ ID NO: 2220.

In one embodiment, the fusion set of primers comprises the pairs of primers having the nucleic acid sequence SEQ ID NO: 1933 to SEQ ID NO: 2166 and optionally of SEQ ID NO: 2167 to SEQ ID NO: 2184 and/or SEQ ID NO: 2185 to SEQ ID NO: 2202 and/or SEQ ID NO: 2203 to SEQ ID NO: 2220.

In a preferred embodiment, the fusion set of primers comprises the pairs of primers able to produce the amplicons having the nucleic acid sequence SEQ ID NO: 1869 to SEQ ID NO: 2220.

In one embodiment, the fusion set of primers consists of the pairs of primers able to produce the amplicons having the nucleic acid sequence SEQ ID NO: 1933 to SEQ ID NO: 2166 and optionally of SEQ ID NO: 2167 to SEQ ID NO: 2184 and/or SEQ ID NO: 2185 to SEQ ID NO: 2202 and/or SEQ ID NO: 2203 to SEQ ID NO: 2220.

In a preferred embodiment, the fusion set of primers consists of the pairs of primers able to produce the amplicons having the nucleic acid sequence SEQ ID NO: 1869 to SEQ ID NO: 2220.

In one embodiment, the composition of primers for HPV typing comprises the pairs of primers having the nucleic acid sequence SEQ ID NO: 1-2 to SEQ ID NO: 1439-1440.

In one embodiment, the composition of primers for HPV typing consists of the pairs of primers able to produce amplicons having the nucleic acid sequence SEQ ID NO: 1-2 to SEQ ID NO: 1439-1440.

In one embodiment, the composition of primers for HPV typing comprises the pairs of primers able to produce amplicons having the nucleic acid sequence SEQ ID NO: 1501 to SEQ ID NO: 2220.

In one embodiment, the composition of primers for HPV typing consists of the pairs of primers having the nucleic acid sequence SEQ ID NO: 1501 to SEQ ID NO: 2220.

The primers of the human set of primers target human sequences.

The human set of primers may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30, or 2, 3, 4, 5, 6, 7, 8, 9, 10, 11, 12, 13, 14, 15, 16, 17, 18, 19, 20, 21, 22, 23, 24, 25, 26, 27, 28, 29 or 30 pairs of primers having the nucleic acid sequence selected from the group consisting of SEQ ID NO: 1441-1442, SEQ ID NO: 1443-1444, SEQ ID NO: 1445-1446, SEQ ID NO: 1447-1448, SEQ ID NO: 1449-1450, SEQ ID NO: 1451-1452, SEQ ID NO: 1453-1454, SEQ ID NO: 1455-1456, SEQ ID NO: 1457-1458, SEQ ID NO: 1459-1460, SEQ ID NO: 1461-1462, SEQ ID NO: 1463-1464, SEQ ID NO: 1465-1466, SEQ ID NO: 1467-1468, SEQ ID NO: 1469-1470, SEQ ID NO: 1471-1472, SEQ ID NO: 1473-1474, SEQ ID NO: 1475-1476, SEQ ID NO: 1477-1478, SEQ ID NO: 1479-1480, SEQ ID NO: 1481-1482, SEQ ID NO: 1483-1484, SEQ ID NO: 1485-1486, SEQ ID NO: 1487-1488, SEQ ID NO: 1489-1490, SEQ ID NO: 1491-1492, SEQ ID NO: 1493-1494, SEQ ID NO: 1495-1496, SEQ ID NO: 1497-1498 and SEQ ID NO: 1499-1500.

In one preferred embodiment, the human set of primers comprises SEQ ID NO: 1441-1442 to SEQ ID NO: 1499-1500.

In one more preferred embodiment, the human set of primers consists SEQ ID NO: 1441-1442 to SEQ ID NO: 1499-1500.

The human set of primers may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30, or 2, 3, 4, 5, 6, 7, 8, 9, 10, 11, 12, 13, 14, 15, 16, 17, 18, 19, 20, 21, 22, 23, 24, 25, 26, 27, 28, 29 or 30 pairs of primers able to produce the amplicons having the nucleic acid sequence selected from the group consisting of SEQ ID NO: 2221 to SEQ ID NO: 2250.

In one preferred embodiment, the human set of primers comprises pairs of primers able to produce the amplicons having the nucleic acid sequence SEQ ID NO: 2221 to SEQ ID NO: 2250.

In one more preferred embodiment, the human set of primers consists of pairs of primers able to produce the amplicons having the nucleic acid sequence SEQ ID NO: 2221 to SEQ ID NO: 2250.

In one embodiment, the composition of primers for HPV typing comprises the pairs of primers having the nucleic acid sequence SEQ ID NO: 1-2 to SEQ ID NO: 1499-1500.

Due to the redondancy between the pairs of primers the nucleic acid sequence SEQ ID NO: 1-2 to SEQ ID NO: 1499-1500 represent only 525 unique pairs of primers.

In one embodiment, the composition of primers for HPV typing consists of the pairs of primers able to produce amplicons having the nucleic acid sequence SEQ ID NO: 1-2 to SEQ ID NO: 1499-1500.

In one embodiment, the composition of primers for HPV typing comprises the pairs of primers able to produce amplicons having the nucleic acid sequence SEQ ID NO: 1501 to SEQ ID NO: 2250.

In one embodiment, the composition of primers for HPV typing consists of the pairs of primers having the nucleic acid sequence SEQ ID NO: 1501 to SEQ ID NO: 2250.

The present invention also relates to a kit for HPV typing comprising the composition of primers for HPV typing of the invention and optionally reagents for cDNA amplification.

The reagents for the kit for HPV typing may be the same as those for detecting HSIL.

The present invention also relates to the use of the composition of primers for HPV typing as defined above or of the kit for HPV typing as defined above for HPV typing.

An In Vitro Method for HPV Typing in a Biological Sample

The present invention also relates to an in vitro method for HPV typing in a biological sample comprising the steps of:
(a) extraction of RNA from the biological sample,
(b) reverse transcription of the RNA so as to generate cDNA,
(c) amplification of the cDNA generated at step (b) with the composition of primers for HPV typing so as to produce amplicons,
(d) quantification of the expression level of each amplicon.

The quantification of the expression level of each amplicon as well as the step (a) to (c) may be carried by the same methods as those disclosed for the in vitro method for detecting HSIL.

Typically, the in vitro method for HPV typing in a biological sample further comprises the step (e) of for each HPV type, comparing the expression level of all amplicons specific of said HPV type with a reference value, wherein if the expression level of all the amplicons specific of said HPV type is higher than the reference value, it is indicative of the presence of said HPV type in the biological sample.

Indeed, the number of reads mapping to HPV-specific amplicons (i.e. the sum of categories “splice junction”, “unsplice junction” and “genomic”) was used to detect the presence of a given HPV genotype. According the results of the inventors, the reference value is preferably between of 100-200 reads, more preferably 150 reads.

The practice of the present invention will employ, unless otherwise indicated, conventional techniques, which are within the skill of the art. Such techniques are explained fully in the literature.

For a better understanding of the invention and to show how the same may be carried into effect, there will now be described by way of example a specific mode contemplated by the Inventors with reference to the accompanying drawings in which:

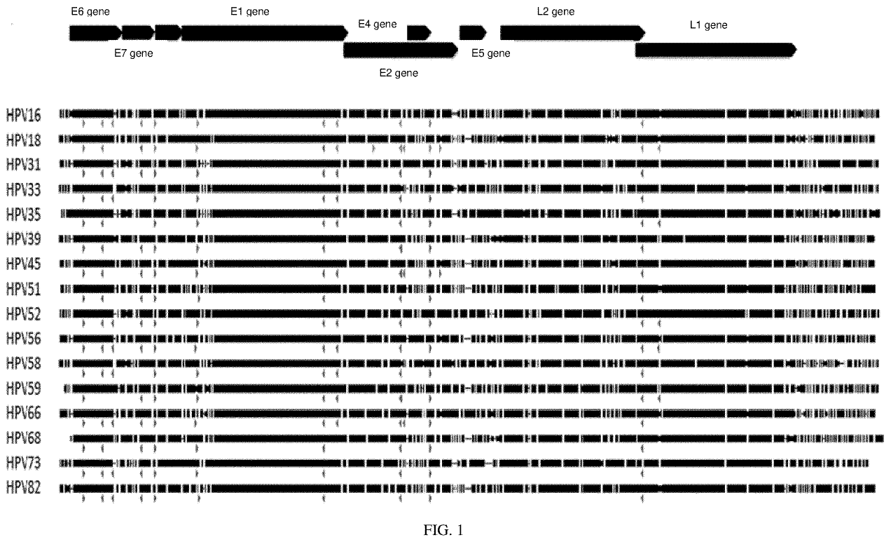

FIG. 1 shows an alignment of the genomes of HPV wherein are located the known and predictive splice donor and splice acceptor sites.

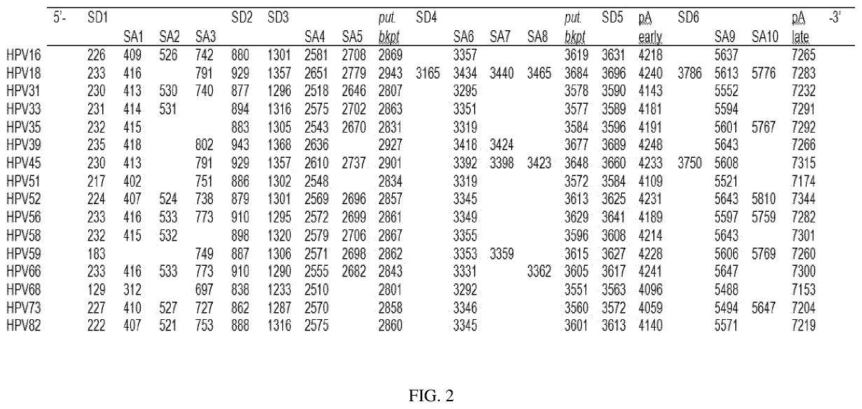

FIG. 2 shows the location on the genomes of HPV and predictive splice donor (SD) and splice acceptor (SA) sites.

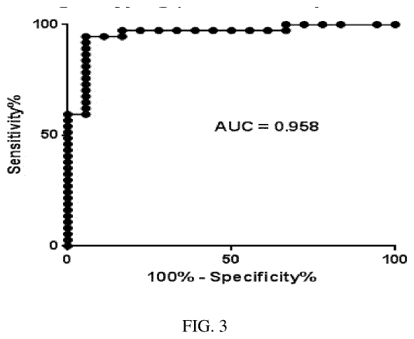

FIG. 3 shows a Receiver Operating Characteristic (ROC) curve. HPV DNA (PapilloCheck) was used as a reference to evaluate the performances of HPV RNA-Seq for HPV genotyping applications. AUC means Area Under Curve.

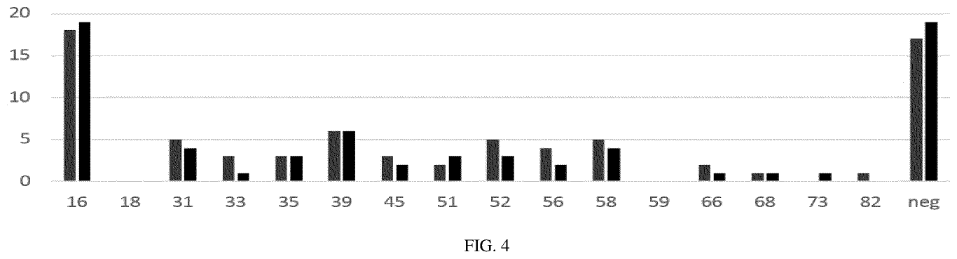

FIG. 4 shows the number of HPV genotypes identified by HPV RNA-Seq (left bars) at threshold value of 150 reads, vs HPV DNA -PapilloCheck (right bars).

The examples and figures should not be interpreted in any way as limiting the scope of the present invention.

### EXAMPLES

Material and Methods:

Evaluation of Transport Medium for RNA Conservation

HPV16-positive cervical squamous cell carcinoma SiHa cells were cultivated and inoculated at a final concentration of 7×10⁴ cells/mL in four transport medium: PreservCyt Solution (Hologic, USA), NovaPrep HQ+ Solution (Novaprep, France), RNA Protect Cell Reagent (Qiagen, Germany) and NucliSens Lysis Buffer (BioMerieux, France). The mixtures were aliquoted in 1 mL tubes and kept at room temperature for 2 hours (DO), 48 hours (D2), 168 hours (D7), 336 hours (D14) and 504 hours (D21). In parallel, 7×10⁴ cells pellets without transport medium were kept frozen −80° C. for 2 hours, 48 hours, 168 hours, 336 hours and 504 hours as a control. At D0, D2, D7, D14 and D21, room temperature aliquots were centrifuged, the medium removed, and the pellets were frozen −80° C. for a short time (<1 h) before proceeding with RNA extraction. In the particular case of the NucliSens Lysis Buffer since the cells were lysed, the entire 1 mL aliquot was frozen −80° C. for a short time without prior centrifugation. For each sample, RNA was extracted using the PicoPure RNA Isolation kit (Thermo Fisher Scientific, USA), together with the corresponding (time match) frozen control, so that all samples have undergone one freezing cycle. RT-qPCR was performed to quantify the expression of the two human genes G6PD (forward primer: TGCAGATGCTGTGTCTGG (SEQ ID NO: 2251); reverse primer: CGTACTGGCCCAGGACC (SEQ ID NO: 2252) and GAPDH (forward primer: GAAGGTGAAGGTCGGAGTC; reverse primer: GAAGATGGTGATGGGATTTC (SEQ ID NO: 2253)) and the expression of the two viral genes HPV16 E6 (forward primer: ATGCACCAAAAGAGAACTGC (SEQ ID NO: 2254); reverse primer: TTACAGCTGGGTTTCTCTAC (SEQ ID NO: 2255)) and E7 (forward primer: GTAACCTTTTGTTGCAAGTGTGACT (SEQ ID NO: 2256); reverse primer: GATTATGGTTTCTGAGAACAGATGG (SEQ ID NO:2257)). RNA integrity was assessed on a Bioanalyzer instrument (Agilent, USA).

HPV Selection and Splice Sites Analysis

HPV reference clones made available by the International Human Papillomavirus Reference Center (Karolinska University, Stockholm, Sweden) served as reference genomes, except for HPV68 which was retrieved from Chen et al. (Evolution and Taxonomic Classification of Alphapapillomavirus 7 Complete Genomes: HPV18, HPV39, HPV45, HPV59, HPV68 and HPV70. PLOS ONE, 2013, 8:e72565). Accession numbers used in this study were: K02718 (HPV16), X05015 (HPV18), J04353 (HPV31), M12732 (HPV33), X74477 (HPV35), M62849 (HPV39), X74479 (HPV45), M62877 (HPV51), X74481 (HPV52), X74483 (HPV56), D90400 (HPV58), X77858 (HPV59), U31794 (HPV66), KC470267 (HPV68), X94165 (HPV73) and AB027021 (HPV82). Multiple alignments of HPV genomes was done with ClustalW v2.1 using Geneious v10 (Kearse M. et al. Geneious Basic: an integrated and extendable desktop software platform for the organization and analysis of sequence data. Bioinforma Oxf Engl, 2012, 28:1647-9). Previously known splice donor (SD) and splice acceptor (SA) sites for HPV16 (Zheng Z-M, et al. Papillomavirus genome structure, expression, and post-transcriptional regulation. Front Biosci J Virtual Libr, 2006, 11:2286-302) and HPV18 (Wang X, et al. Construction of a full transcription map of human papillomavirus type 18 during productive viral infection. J Virol, 2011, 85:8080-92) were reported on the alignment, and predictions of unknown SD and SA sites were done manually for the other genotypes by sequence analogy (FIGS. 1 and 2).

### HPV RNA-Seq AmpliSeq Custom Panel

A custom AmpliSeq panel was designed to be used on both PGM and Ion Proton instruments (Thermo Fisher Scientific). Five categories of target sequences were defined as follow:

HPV splice junctions (sp): a set of target sequences which are specific HPV splice events, involving a pair of splice donor (SD) and splice acceptor (SA) sites. The nomenclature includes a “sp” tag. For example, “31_sp_1296_3295_J43-46” stands for HPV31 (31), splice junction (sp), SD at position 1296 on HPV31 genome, SA at position 3295 on HPV31 genome, and junction (J) at position 43-46 on amplicon. The junction coordinates are given in a 4-bases interval, where the first 2 bases correspond to the donor part (or left part) and the last 2 bases to the acceptor part (or right part) of the sequence. Primers and amplicons corresponding to splice junctions set are given at Table 2A and 2Abis.

HPV unsplice junctions (unsp): a set of target sequences which are specific HPV genomic regions spanning either SD or SA sites, in the absence of any splice event. The nomenclature includes an “unsp” tag. For example, “31_unsp_1296_1297_J43-46” stands for HPV31 (31), unspliced (unsp), last base of the left part of the amplicon at position 1296 on HPV31 genome, first base of the right part of the amplicon at position 1297 on HPV31 genome, junction (J) at position 43-46 on amplicon. Primers and amplicons corresponding to unsplice junctions set are given at Table 2B and 2Bbis. In this context, the term ‘junction’ refers to the exon-intron interface (i.e. the position where a donor or acceptor site would be found in case of a splice event), and the associated junction coordinates are used to characterize unspliced sequences bioinformatically as described in section “Sequencing data processing”.

HPV genome away from splice junctions (gen): a set of target sequences which are specific HPV genomic regions, away from any SD or SA sites. The nomenclature includes a “gen” tag. For example, “45_gen_1664_1794_NoJ” stands for HPV45 (45), HPV genomic region (gen), amplicon coordinates from position 1664 to position 1794 on HPV45 genome. Primers and amplicons corresponding to the genomic set are given at Table 2C and 2Cbis.

HPV-human fusion sequences (fus): a set of hypothesis-driven viral-cellular fusion transcripts, based on previous descriptions (Wentzensen N, et al. Characterization of viral-cellular fusion transcripts in a large series of HPV16 and 18 positive anogenital lesions. Oncogene, 2002, 21:419-2622-26, Tang K-W, et al. The landscape of viral expression and host gene fusion and adaptation in human cancer. Nat Commun, 2013, 4:2513, Peter M, et al. MYC activation associated with the integration of HPV DNA at the MYC locus in genital tumors. Oncogene, 2006, 25:5985-93, Lu X, et al. Multiple-integrations of HPV16 genome and altered transcription of viral oncogenes and cellular genes are associated with the development of cervical cancer. PloS One, 2014, 9:e97588, Kraus I, et al. The majority of viral-cellular fusion transcripts in cervical carcinomas cotranscribe cellular sequences of known or predicted genes. Cancer Res, 2008, 68:2514-22). For each HPV, 18 fusion sequence candidates involving SA2 or putative breakpoint 1 or 2 (put. bkpt, see FIG. 2) for the viral part, and specific exons from MYC or PVT1 oncogenes for the cellular part, were added to the design. For example, “18_fus_929_MYC_001_exon3_J37-40” stands for HPV18 (18), candidate fusion transcript (fus), break/fusion at position 929 on HPV18 genome, fused with MYC mRNA isoform 001 exon 3, junction (J) at position 37-40 on amplicon. Primers and amplicons corresponding to the fusion set are given at Table 2D and 2Dbis.

Human sequences (hg): a set of 30 human sequences used as internal controls retrieved from publically available AmpliSeq projects and representing housekeeping genes (ACTB, B2M, GAPDH, GUSB, RPLPO), epithelial markers (KRT10, KRT14, KRT17), oncogenes, tumor supressor genes canonical cancer pathways and direct or indirect downstream effectors of HPV oncoproteins (AKT1, BCL2, BRAF, CDH1, CDKN2A, CDKN2B, ERBB2, FOS, HRAS, KRAS, MET, MK167, MYC, NOTCH1, PCNA, PTEN, RB1, STAT1, TERT, TOP2A, TP53, WNT1). The nomenclature for these sequences includes an “hg” tag. For example, “hg_TOP2A_E21E22” stands for human topoisomerase 2A mRNA exon (Wang X, et al. 2011, Wentzensen N, et al., 2002). Primers and amplicons corresponding to the human set are given at Table 2E and 2Ebis.

In total, 750 target sequences were included into the panel (Table 1) and can be amplified with a pool of 525 unique primers (Table 2A-2E). The average amplicon size of the panel (primers included) is 141 bp (range: 81-204 bp). A detailed table including the nucleic acid sequences of the primers along with their corresponding amplicons and amplicon sequences is given in Table 2Abis-2Ebis.

Table 1 below shows the HPV RNA-Seq AmpliSeq custom panel contents. The number of target amplicons is indicated for each category (sp, unsp, gen, fus, hg) and for each viral and cellular origin. Putative high-risk HPV are indicated by a star (*).

TABLE 1

| | Sp | unsp | gen | fus | Hg | |
|---|---|---|---|---|---|---|
| HPV16 | 14 | 11 | 4 | 18 | 0 | |
| HPV18 | 18 | 12 | 4 | 18 | 0 | |
| HPV31 | 14 | 11 | 4 | 18 | 0 | |
| HPV33 | 13 | 9 | 4 | 18 | 0 | |
| HPV35 | 14 | 10 | 4 | 18 | 0 | |
| HPV39 | 10 | 8 | 4 | 18 | 0 | |
| HPV45 | 14 | 10 | 4 | 18 | 0 | |
| HPV51 | 10 | 9 | 4 | 18 | 0 | |
| HPV52 | 16 | 11 | 4 | 18 | 0 | |
| HPV56 | 16 | 10 | 4 | 18 | 0 | |
| HPV58 | 13 | 8 | 4 | 18 | 0 | |
| HPV59 | 14 | 8 | 4 | 18 | 0 | |
| HPV66 | 15 | 8 | 4 | 18 | 0 | |
| HPV68* | 10 | 9 | 4 | 18 | 0 | |
| HPV73* | 13 | 10 | 4 | 18 | 0 | |
| HPV82* | 11 | 9 | 4 | 18 | 0 | |
| human | 0 | 0 | 0 | 0 | 30 | |
| TOTAL | 215 | 153 | 64 | 288 | 30 | 750 |

TABLE 2A

| | | | Forward | | Reverse |
|---|---|---|---|---|---|
| | | | primer | | primer |
| HPV | Splice | Forward primer | SEQ | Reverse primer | SEQ |
| type | junction | nucleic acid sequence | ID NO | nucleic acid sequence | ID NO |
| HPV16 | SD3-SA4 | GCGGGTATGGCAATACTGAAGT | 1 | GTTTTCGTCAAATGGAAACTCATTAGGA | 2 |
| | i = 1 | | | | |
| HPV16 | SD3-SA5 | GCGGGTATGGCAATACTGAAGT | 3 | TGACACACATTTAAACGTTGGCAAAG | 4 |
| | i = 2 | | | | |
| HPV16 | SD3-SA6 | GCGGGTATGGCAATACTGAAGT | 5 | AAGGCGACGGCTTTGGTAT | 6 |
| | i = 3 | | | | |
| HPV16 | SD1-SA4 | CACAGAGCTGCAAACAACTATACAT | 7 | GTTTTCGTCAAATGGAAACTCATTAGGA | 8 |
| | i = 4 | | | | |
| HPV16 | SD1-SA5 | CACAGAGCTGCAAACAACTATACAT | 9 | TGACACACATTTAAACGTTGGCAAAG | 10 |
| | i = 5 | | | | |
| HPV16 | SD1-SA6 | CACAGAGCTGCAAACAACTATACAT | 11 | AAGGCGACGGCTTTGGTAT | 12 |
| | i = 6 | | | | |
| HPV16 | SD1-SA1 | CACAGAGCTGCAAACAACTATACAT | 13 | TGTCCAGATGTCTTTGCTTTTCTTCA | 14 |
| | I = 7 | | | | |
| HPV16 | SD1-SD2 | CACAGAGCTGCAAACAACTATACAT | 15 | TCAGTTGTCTCTGGTTGCAAATCT | 16 |
| | i = 8 | | | | |
| HPV16 | SD1-SA3 | CACAGAGCTGCAAACAACTATACAT | 17 | CCATTAACAGGTCTTCCAAAGTACGA | 18 |
| | i = 9 | | | | |
| HPV16 | SD5-SA9 | GCTCACACAAAGGACGGATTAAC | 19 | ATCCGTGCTTACAACCTTAGATACTG | 20 |
| | i = 11 | | | | |
| HPV16 | SD2-SA4 | GGAATTGTGTGCCCCATCTGT | 21 | GTTTTCGTCAAATGGAAACTCATTAGGA | 22 |
| | i = 15 | | | | |
| HPV16 | SD2-SA5 | GGAATTGTGTGCCCCATCTGT | 23 | TGACACACATTTAAACGTTGGCAAAG | 24 |
| | i = 16 | | | | |
| HPV16 | SD2-SA6 | GGAATTGTGTGCCCCATCTGT | 25 | AAGGCGACGGCTTTGGTAT | 26 |
| | i = 17 | | | | |
| HPV16 | SD2-SA9 | GGAATTGTGTGCCCCATCTGT | 27 | ATCCGTGCTTACAACCTTAGATACTG | 28 |
| | i = 18 | | | | |
| HPV18 | SD3-SA4 | TCAGATAGTGGCTATGGCTGTTCT | 29 | GTCATTTATTTCATATACTGGATTGCCA | 30 |
| | i = 1 | | | | |
| HPV18 | SD3-SA5 | TCAGATAGTGGCTATGGCTGTTCT | 31 | GGTTTCCTTCGGTGTCTGCAT | 32 |
| | i = 2 | | | | |
| HPV18 | SD3-SA6 | TCAGATAGTGGCTATGGCTGTTCT | 33 | ACGTCTGGCCGTAGGTCT | 34 |
| | i = 3 | | | | |
| HPV18 | SD1-SA4 | TTCACTGCAAGACATAGAAATAACCTGT | 35 | GTCATTTATTTCATATACTGGATTGCCA | 36 |
| | i = 4 | | | | |
| HPV18 | SD1-SA5 | TTCACTGCAAGACATAGAAATAACCTGT | 37 | GGTTTCCTTCGGTGTCTGCAT | 38 |
| | i = 5 | | | | |
| HPV18 | SD1-SA6 | TTCACTGCAAGACATAGAAATAACCTGT | 39 | ACGTCTGGCCGTAGGTCT | 40 |
| | i = 6 | | | | |
| HPV18 | SD1-SA1 | TTCACTGCAAGACATAGAAATAACCTGT | 41 | CCCAGCTATGTTGTGAAATCGT | 42 |
| | i = 7 | | | | |
| HPV18 | SD1-SD2 | TTCACTGCAAGACATAGAAATAACCTGT | 43 | AGAAACAGCTGCTGGAATGCT | 44 |
| | i = 9 | | | | |
| HPV18 | SD4-SA6 | GGATTGGACACTGCAAGACACA | 45 | ACGTCTGGCCGTAGGTCT | 46 |
| | i = 10 | | | | |
| HPV18 | SD5-SA9 | CAGCTACACCTACAGGCAACAA | 47 | GTATTTACAACTCTTGCCACAGAAGG | 48 |
| | i = 11 | | | | |
| HPV18 | SD5-SD10 | CAGCTACACCTACAGGCAACAA | 49 | TCAGGTAACTGCACCCTAAATACTCTAT | 50 |
| | i = 12 | | | | |
| HPV18 | SD6-SA9 | CGAAAACATAGCGACCACTATAGAGAT | 51 | GTATTTACAACTCTTGCCACAGAAGGA | 52 |
| | i = 13 | | | | |
| HPV18 | SD6-SA10 | CGAAAACATAGCGACCACTATAGAGAT | 53 | TCAGGTAACTGCACCCTAAATACTCTAT | 54 |
| | i = 14 | | | | |
| HPV18 | SD2-SA4 | TGCATCCCAGCAGTAAGCAA | 55 | GTCATTTATTTCATATACTGGATTGCCA | 56 |
| | i = 15 | | | | |
| HPV18 | SD2-SA5 | TGCATCCCAGCAGTAAGCAA | 57 | GGTTTCCTTCGGTGTCTGCAT | 58 |
| | i = 16 | | | | |
| HPV18 | SD2-SA8 | TGCATCCCAGCAGTAAGCAA | 59 | ACGTCTGGCCGTAGGTCT | 60 |
| | i = 25 | | | | |
| HPV18 | SD2-SA9 | TGCATCCCAGCAGTAAGCAA | 61 | GTATTTACAACTCTTGCCACAGAAGGA | 62 |
| | i = 18 | | | | |
| HPV18 | SD2-SA10 | TGCATCCCAGCAGTAAGCAA | 63 | TCAGGTAACTGCACCCTAAATACTCTAT | 64 |
| | i = 19 | | | | |
| HPV31 | SD3-SA4 | GCGGGTATGGCAATACTGAAGT | 65 | AATGTAAAAACCACCAGTCTGCTATGTA | 66 |
| | i = 1 | | | | |
| HPV31 | SD3-SA5 | GCGGGTATGGCAATACTGAAGT | 67 | CGTTGAGAAAGAGTCTCCATCGTTTT | 68 |
| | i = 2 | | | | |
| HPV31 | SD3-SA6 | GCGGGTATGGCAATACTGAAGT | 69 | GAATTCGATGTGGTGGTGTTGTTG | 70 |
| | i = 3 | | | | |
| HPV31 | SD1-SA4 | CGGCATTGGAAATACCCTACGAT | 71 | AATGTAAAAACCACCAGTCTGCTATGTA | 72 |
| | i = 4 | | | | |
| HPV31 | SD1-SA5 | CGGCATTGGAAATACCCTACGAT | 73 | CGTTGAGAAAGAGTCTCCATCGTTTT | 74 |
| | i = 5 | | | | |
| HPV31 | SD1-SA6 | CGGCATTGGAAATACCCTACGAT | 75 | GAATTCGATGTGGTGGTGTTGTTG | 76 |
| | i = 6 | | | | |
| HPV31 | SD1-SA1 | CGGCATTGGAAATACCCTACGAT | 77 | TTTTCTTCTGGACACAACGGTCTT | 78 |
| | i = 7 | | | | |
| HPV31 | SD1-SA2 | CGGCATTGGAAATACCCTACGAT | 79 | ACATAGTCTTGCAACGTAGGTGTTT | 80 |
| | i = 8 | | | | |
| HPV31 | SD1-SA3 | CGGCATTGGAAATACCCTACGAT | 81 | CATTAACAGCTCTTGCAATATGCGAATA | 82 |
| | i = 9 | | | | |
| HPV31 | SD5-SA9 | CAGCTGCATGCACAAACCA | 83 | TTTAGACACTGGGACAGGTGGTA | 84 |
| | i = 11 | | | | |
| HPV31 | SD2-SA5 | AATCGTGTGCCCCAACTGT | 85 | AATGTAAAAACCACCAGTCTGCTATGTA | 86 |
| | i = 15 | | | | |
| HPV31 | SD2-SA5 | AATCGTGTGCCCCAACTGT | 87 | CGTTGAGAAAGAGTCTCCATCGTTTT | 88 |
| | i = 16 | | | | |
| HPV31 | SD2-SA6 | AATCGTGTGCCCCAACTGT | 89 | GAATTCGATGTGGTGGTGTTGTTG | 90 |
| | i = 17 | | | | |
| HPV31 | SD2-SA9 | AATCGTGTGCCCCAACTGT | 91 | TTTAGACACTGGGACAGGTGGTA | 92 |
| | i = 18 | | | | |
| HPV33 | SD3-SA4 | GATGAGCTAGAAGACAGCGGATATG | 93 | CATACACTGGGTTACCATTTTCATCAAA | 94 |
| | i = 1 | | | | |
| HPV33 | SD3-SA5 | GATGAGCTAGAAGACAGCGGATATG | 95 | TGATATTTCCTCCATGGTTTTCCTTGTC | 96 |
| | i = 2 | | | | |
| HPV33 | SD3-SA6 | GATGAGCTAGAAGACAGCGGATATG | 97 | GTGGTGGTCGGTTATCGTTGT | 98 |
| | i = 3 | | | | |
| HPV33 | SD1-SA4 | AGCATTGGAGACAACTATACACAACATT | 99 | CATACACTGGGTTACCATTTTCATCAAA | 100 |
| | i = 4 | | | | |
| HPV33 | SD1-SA5 | AGCATTGGAGACAACTATACACAACATT | 101 | TGATATTTCCTCCATGGTTTTCCTTGTC | 102 |
| | i = 5 | | | | |
| HPV33 | SD1-SA6 | AGCATTGGAGACAACTATACACAACATT | 103 | GTGGTGGTCGGTTATCGTTGT | 104 |
| | i = 6 | | | | |
| HPV33 | SD1-SA1 | AGCATTGGAGACAACTATACACAACATT | 105 | TCGTTTGTTTAAATCCACATGTCGTTTT | 106 |
| | i = 7 | | | | |
| HPV33 | SD1-SA2 | AGCATTGGAGACAACTATACACAACATT | 107 | CATATTCCTTTAACGTTGGCTTGTGT | 108 |
| | i = 8 | | | | |
| HPV33 | SD5-SA9 | ACGTACTGCAACTAACTGCACAA | 109 | ATCAGTGCTGACAACTTTAGATACAGG | 110 |
| | i = 11 | | | | |
| HPV33 | SD2-SA4 | GTGCCCTACCTGTGCACAA | 111 | CATACACTGGGTTACCATTTTCATCAAA | 112 |
| | i = 15 | | | | |
| HPV33 | SD2-SA5 | GTGCCCTACCTGTGCACAA | 113 | TGATATTTCCTCCATGGTTTTCCTTGTC | 114 |
| | i = 16 | | | | |
| HPV33 | SD2-SA6 | GTGCCCTACCTGTGCACAA | 115 | GTGGTGGTCGGTTATCGTTGT | 116 |
| | i = 17 | | | | |
| HPV33 | SD2-SA9 | GTGCCCTACCTGTGCACAA | 117 | ATCAGTGCTGACAACTTTAGATACAGG | 118 |
| | i = 18 | | | | |
| HPV35 | SD3-SA4 | ATTATTTGAACTACCAGACAGCGGTT | 119 | TCATTGTGAAATGTAAAGACCACTACCC | 120 |
| | i = 1 | | | | |
| HPV35 | SD3-SA5 | ATTATTTGAACTACCAGACAGCGGTT | 121 | GGAAAGCGTCTCCATCATTTTCTTTG | 122 |
| | i = 2 | | | | |
| HPV35 | SD3-SA6 | ATTATTTGAACTACCAGACAGCGGTT | 123 | GCTTTGGTATGGGTCTCGGT | 124 |
| | i = 3 | | | | |
| HPV35 | SD1-SA4 | CGAGGTAGAAGAAAGCATCCATGAAAT | 125 | TCATTGTGAAATGTAAAGACCACTACCC | 126 |
| | i = 4 | | | | |
| HPV35 | SD1-SA5 | CGAGGTAGAAGAAAGCATCCATGAAAT | 127 | GGAAAGCGTCTCCATCATTTTCTTTG | 128 |
| | i = 5 | | | | |
| HPV35 | SD1-SA6 | CGAGGTAGAAGAAAGCATCCATGAAAT | 129 | GCTTTGGTATGGGTCTCGGT | 130 |
| | i = 6 | | | | |
| HPV35 | SD1-SA1 | CGAGGTAGAAGAAAGCATCCATGAAAT | 131 | TCCACCGATGTTATGGAATCGTTTT | 132 |
| | i = 7 | | | | |
| HPV35 | SD5-SA9 | TCTACATCTGACTGCACAAACAAAGA | 133 | CATCAGTGCTAACAACCTTAGACACT | 134 |
| | i = 11 | | | | |
| HPV35 | SD5-SA10 | TCTACATCTGACTGCACAAACAAAGA | 135 | ACTCTGTATTGCAAACCAGATACCTTG | 136 |
| | i = 12 | | | | |
| HPV35 | SD2-SA4 | CGGCTGTTCACAGAGAGCATAAT | 137 | TCATTGTGAAATGTAAAGACCACTACCC | 138 |
| | i = 14 | | | | |
| HPV35 | SD2-SA5 | CGGCTGTTCACAGAGAGCATAAT | 139 | GGAAAGCGTCTCCATCATTTTCTTTG | 140 |
| | i = 16 | | | | |
| HPV35 | SD2-SA6 | CGGCTGTTCACAGAGAGCATAAT | 141 | GCTTTGGTATGGGTCTCGGT | 142 |
| | i = 17 | | | | |
| HPV35 | SD2-SA9 | CGGCTGTTCACAGAGAGCATAAT | 143 | CATCAGTGCTAACAACCTTAGACACT | 144 |
| | i = 18 | | | | |
| HPV35 | SD2-SA10 | CGGCTGTTCACAGAGAGCATAAT | 145 | ACTCTGTATTGCAAACCAGATACCTTG | 146 |
| | i = 19 | | | | |
| HPV39 | SD3-SA4 | GGTGTATTCCGTGCCAGACA | 147 | CTGTTTTGGTCAAATGGAAATGCATTAG | 148 |
| | i = 1 | | | | |
| HPV39 | SD3-SA7 | GGTGTATTCCGTGCCAGACA | 149 | GGTCGCGGTGGTGTTTGATAA | 150 |
| | i = 22 | | | | |
| HPV39 | SD1-SA4 | CACCACCTTGCAGGACATTACAATA | 151 | CTGTTTTGGTCAAATGGAAATGCATTAG | 152 |
| | i = 4 | | | | |
| HPV39 | SD1-SA1 | CACCACCTTGCAGGACATTACAATA | 153 | GGTCGCGGTGGTGTTTGATAA | 154 |
| | i = 20 | | | | |
| HPV39 | SD1-SA3 | CACCACCTTGCAGGACATTACAATA | 155 | CTGTCCTGTATAGCTTCCTGCTATTTT | 156 |
| | i = 7 | | | | |
| HPV39 | SD5-SA9 | CACCACCTTGCAGGACATTACAATA | 157 | TGCTGTAGTTGTCGCAGAGTATC | 158 |
| | i = 9 | | | | |
| HPV39 | SD2-SA4 | CACAGTAACAGTACAGGCCACA | 159 | AGTATTGACAACCTTCGCCACA | 160 |
| | i = 11 | | | | |
| HPV39 | SD2-SA7 | CGTGGTGTGCAACTGCAA | 161 | CTGTTTTGGTCAAATGGAAATGCATTAG | 162 |
| | i = 15 | | | | |
| HPV39 | SD2-SA7 | CGTGGTGTGCAACTGCAA | 163 | GGTCGCGGTGGTGTTTGATAA | 164 |
| | i = 21 | | | | |
| HPV39 | SD2-SA9 | CGTGGTGTGCAACTGCAA | 165 | AGTATTGACAACCTTCGCCACA | 166 |
| | i = 18 | | | | |
| HPV45 | SD3-SA4 | TCAGATAGTGGCTATGGCTGTTCT | 167 | GAAATGCATGTGGAAATGTAAATACCGT | 168 |
| | i = 1 | | | | |
| HPV45 | SD3-SA5 | TCAGATAGTGGCTATGGCTGTTCT | 169 | GGATTCCTTCGGTGTCTGCAT | 170 |
| | i = 2 | | | | |
| HPV45 | SD3-SA8 | TCAGATAGTGGCTATGGCTGTTCT | 171 | CCCACGGATGCGGTTTTG | 172 |
| | i = 23 | | | | |
| HPV45 | SD1-SA4 | CTACAAGACGTATCTATTGCCTGTGT | 173 | GAAATGCATGTGGAAATGTAAATACCGT | 174 |
| | i = 4 | | | | |
| HPV45 | SD1-SA5 | CTACAAGACGTATCTATTGCCTGTGT | 175 | GGATTCCTTCGGTGTCTGCAT | 176 |
| | i = 5 | | | | |
| HPV45 | SD1-SA8 | CTACAAGACGTATCTATTGCCTGTGT | 177 | CCCACGGATGCGGTTTTG | 178 |
| | i = 24 | | | | |
| HPV45 | SD1-SA1 | CTACAAGACGTATCTATTGCCTGTGT | 179 | CGTTTGTCCTTAAGGTGTCTACGTTTT | 180 |
| | i = 7 | | | | |
| HPV45 | SD1-SA3 | CTACAAGACGTATCTATTGCCTGTGT | 181 | TCAAAAACAGCTGCTGTAGTGTTCT | 182 |
| | i = 9 | | | | |
| HPV45 | SD5-SA9 | TCCTGTGTTCAAGTACAAGTAACAACAA | 183 | GCTGACAACTCTGGCCACA | 184 |
| | i = 11 | | | | |
| HPV45 | SD6-SA9 | CGCAAATATGCAGACCATTACTCAGAA | 185 | GCTGACAACTCTGGCCACA | 186 |
| | i = 13 | | | | |
| HPV45 | SD2-SA4 | AGCACCTTGTCCTTTGTGTGT | 187 | GAAATGCATGTGGAAATGTAAATACCGT | 188 |
| | i = 15 | | | | |
| HPV45 | SD2-SA5 | AGCACCTTGTCCTTTGTGTGT | 189 | GGATTCCTTCGGTGTCTGCAT | 190 |
| | i = 16 | | | | |
| HPV45 | SD2-SA8 | AGCACCTTGTCCTTTGTGTGT | 191 | CCCACGGATGCGGTTTTG | 192 |
| | i = 25 | | | | |
| HPV45 | SD2-SA9 | AGCACCTTGTCCTTTGTGTGT | 193 | GCTGACAACTCTGGCCACA | 194 |
| | i = 18 | | | | |
| HPV51 | SD3-SA4 | CGGACAGCGGATATGGCAATA | 195 | TCATTCAATGTATACACAGCATTCCCAT | 196 |
| | i = 1 | | | | |
| HPV51 | SD3-SA6 | CGGACAGCGGATATGGCAATA | 197 | CCACGCAGGTGGTAAGGG | 198 |
| | i = 3 | | | | |
| HPV51 | SD1-SA4 | CTGCATGAATTATGTGAAGCTTTGAAC | 199 | TCATTCAATGTATACACAGCATTCCCAT | 200 |
| | i = 4 | | | | |
| HPV51 | SDA-SA6 | CTGCATGAATTATGTGAAGCTTTGAAC | 201 | CCACGCAGGTGGTAAGGG | 202 |
| | i = 6 | | | | |
| HPV51 | SD1-SA1 | CTGCATGAATTATGTGAAGCTTTGAAC | 203 | TCCCGCTATTTCATGGAACCTTTT | 204 |
| | i = 7 | | | | |
| HPV51 | SD1-SA3 | CTGCATGAATTATGTGAAGCTTTGAAC | 205 | CATCTGCTGTACAACGCGAAG | 206 |
| | I = 9 | | | | |
| HPV51 | SD5-SA9 | CTAACACTGGAGGGCACCAAA | 207 | CAATTCGAGACACAGGTGCAG | 208 |
| | i = 11 | | | | |
| HPV51 | SD2-SA4 | GGGCGAACTAAGCCTGGTTT | 209 | TCATTCAATGTATACACAGCATTCCCAT | 210 |
| | i = 15 | | | | |
| HPV51 | SD2-SA6 | GGGCGAACTAAGCCTGGTTT | 211 | CCACGCAGGTGGTAAGGG | 212 |
| | i = 17 | | | | |
| HPV51 | SD2-SA9 | GGGCGAACTAAGCCTGGTTT | 213 | CAATTCGAGACACAGGTGCAG | 214 |
| | i = 18 | | | | |
| HPV52 | SD3-SA4 | CAAACCATGTCACGTAGAAGACAG | 215 | GGGTTTTTGAAATGAAACACAACCAATC | 216 |
| | i = 1 | | | | |
| HPV52 | SD3-SA5 | CAAACCATGTCACGTAGAAGACAG | 217 | CGGTATCGACTCCATCGTTTTCC | 218 |
| | i = 2 | | | | |
| HPV52 | SD3-SA6 | CAAACCATGTCACGTAGAAGACAG | 219 | GCGGAGGTCTTGGAGGTTT | 220 |
| | i = 3 | | | | |
| HPV52 | SD1-SA4 | AGAATCGGTGCATGAAATAAGGCT | 221 | GGGTTTTTGAAATGAAACACAACCAATC | 222 |
| | i = 4 | | | | |
| HPV52 | SD1-SA5 | AGAATCGGTGCATGAAATAAGGCT | 223 | CGGTATCGACTCCATCGTTTTCC | 224 |
| | i = 5 | | | | |
| HPV52 | SD1-SA6 | AGAATCGGTGCATGAAATAAGGCT | 225 | GCGGAGGTCTTGGAGGTTT | 226 |
| | i = 6 | | | | |
| HPV52 | SD1-SA1 | AGAATCGGTGCATGAAATAAGGCT | 227 | CGCTTGTTTGCATTAACATGTCTTTCT | 228 |
| | i = 7 | | | | |
| HPV52 | SD1-SA2 | AGAATCGGTGCATGAAATAAGGCT | 229 | TCAGTTGTTTCAGGTTGCAGATCTAATA | 230 |
| | i = 8 | | | | |
| HPV52 | SD1-SA3 | AGAATCGGTGCATGAAATAAGGCT | 231 | GCATTTGCTGTAGAGTACGAAGGT | 232 |
| | i = 9 | | | | |
| HPV52 | SD5-SA9 | TCACTGCAACTGAGTGCACAA | 233 | TGCTTACAACCTTAGAGACAGGTACA | 234 |
| | i = 11 | | | | |
| HPV52 | SD5-SA10 | TCACTGCAACTGAGTGCACAA | 235 | CCTGTATTGCAGGCCAGACA | 236 |
| | i = 12 | | | | |
| HPV52 | SD2-SA4 | GCTGTTGGGCACATTACAAGTT | 237 | GGGTTTTTGAAATGAAACACAACCAATC | 238 |
| | i = 15 | | | | |
| HPV52 | SD2-SA5 | GCTGTTGGGCACATTACAAGTT | 239 | CGGTATCGACTCCATCGTTTTCC | 240 |
| | i = 16 | | | | |
| HPV52 | SD2-SA6 | GCTGTTGGGCACATTACAAGTT | 241 | GCGGAGGTCTTGGAGGTTT | 242 |
| | i = 17 | | | | |
| HPV52 | SD2-SA9 | GCTGTTGGGCACATTACAAGTT | 243 | TGCTTACAACCTTAGAGACAGGTACA | 244 |
| | i = 18 | | | | |
| HPV52 | SD2-SA10 | GCTGTTGGGCACATTACAAGTT | 245 | CCTGTATTGCAGGCCAGACA | 246 |
| | i = 19 | | | | |
| HPV56 | SD3-SA4 | CAAGACAGCGGGTATGGCAATA | 247 | TGAAACTGAAACACTAACATTCTACTGTGT | 248 |
| | i = 1 | | | | |
| HPV56 | SD3-SA5 | CAAGACAGCGGGTATGGCAATA | 249 | TTTTCTTTGTCCTCGTCGTTATCCAA | 250 |
| | i = 2 | | | | |
| HPV56 | SD3-SA6 | CAAGACAGCGGGTATGGCAATA | 251 | GGTGGTGGTGGTGGTCTT | 252 |
| | i = 3 | | | | |
| HPV56 | SD1-SA4 | GCACCACTTGAGTGAGGTATTAGAA | 253 | TGAAACTGAAACACTAACATTCTACTGTGT | 254 |
| | i = 4 | | | | |
| HPV56 | SD1-SA5 | GCACCACTTGAGTGAGGTATTAGAA | 255 | TTTTCTTTGTCCTCGTCGTTATCCAA | 256 |
| | i = 5 | | | | |
| HPV56 | SD1-SA6 | GCACCACTTGAGTGAGGTATTAGAA | 257 | GGTGGTGGTGGTGGTCTT | 258 |
| | i = 6 | | | | |
| HPV56 | SD1-SA1 | GCACCACTTGAGTGAGGTATTAGAA | 259 | CAATTGCTTTTCCTCCGGAGTTAA | 260 |
| | i = 7 | | | | |
| HPV56 | SD1-SA2 | GCACCACTTGAGTGAGGTATTAGAA | 261 | ACGTCTTGCAGCGTTGGTA | 262 |
| | i = 8 | | | | |
| HPV56 | SD1-SA3 | GCACCACTTGAGTGAGGTATTAGAA | 263 | TGTACAACACGCAGGTCCTC | 264 |
| | i = 9 | | | | |
| HPV56 | SD5-SA9 | ACAACAACCACCCTGGTGATAAG | 265 | ACAACCTTTGAAACAGGTGTTGGA | 266 |
| | i = 11 | | | | |
| HPV56 | SD5-SA10 | ACAACAACCACCCTGGTGATAAG | 267 | CAACCGTACCCTAAATACCCTATATTGA | 268 |
| | i = 12 | | | | |
| HPV56 | SD2-SA4 | GTTAACAGTAACGTGCCCACTCT | 269 | TGAAACTGAAACACTAACATTCTACTGTGT | 270 |
| | i = 15 | | | | |
| HPV56 | SD2-SA5 | GTTAACAGTAACGTGCCCACTCT | 271 | TTTTCTTTGTCCTCGTCGTTATCCAA | 272 |
| | i = 16 | | | | |
| HPV56 | SD2-SA6 | GTTAACAGTAACGTGCCCACTCT | 273 | GGTGGTGGTGGTGGTCTT | 274 |
| | i = 17 | | | | |
| HPV56 | SD2-SA9 | GTTAACAGTAACGTGCCCACTCT | 275 | ACAACCTTTGAAACAGGTGTTGGA | 276 |
| | i = 18 | | | | |
| HPV56 | SD2-SA10 | GTTAACAGTAACGTGCCCACTCT | 277 | CAACCGTACCCTAAATACCCTATATTGA | 278 |
| | i = 19 | | | | |
| HPV58 | SD3-SA4 | AAAATTATTGAGCTAGAAGACAGCGGAT | 279 | TGCATCAAATGGAAATGGATTGTTAAATTCA | 280 |
| | i = 1 | | | | |
| HPV58 | SD3-SA5 | AAAATTATTGAGCTAGAAGACAGCGGAT | 281 | TGATATTTCCTCCATCGTTTTCCTTGTC | 282 |
| | i = 2 | | | | |
| HPV58 | SD3-SA6 | AAAATTATTGAGCTAGAAGACAGCGGAT | 283 | CCCTGTGTACTTTCGTTGTTGGT | 284 |
| | i = 3 | | | | |
| HPV58 | SD1-SA4 | GTCAGGCGTTGGAGACATCT | 285 | TGCATCAAATGGAAATGGATTGTTAAATTCA | 286 |
| | i = 4 | | | | |
| HPV58 | SD1-SA5 | GTCAGGCGTTGGAGACATCT | 287 | TGATATTTCCTCCATCGTTTTCCTTGTC | 288 |
| | i = 5 | | | | |
| HPV58 | SD1-SA6 | GTCAGGCGTTGGAGACATCT | 289 | CCCTGTGTACTTTCGTTGTTGGT | 290 |
| | i = 6 | | | | |
| HPV58 | SD1-SA1 | GTCAGGCGTTGGAGACATCT | 291 | CGACCCGAAATATTATGAAACCTTTTGT | 292 |
| | i = 7 | | | | |
| HPV58 | SD1-SA2 | GTCAGGCGTTGGAGACATCT | 293 | GCGTTGGGTTGTTTCCTCTCA | 294 |
| | i = 8 | | | | |
| HPV58 | SD5-SA9 | GAGGAGGACTACACAGTACAACTAACT | 295 | GCTTACAACCTTAGACACAGGCA | 296 |
| | i = 11 | | | | |
| HPV58 | SD2-SA4 | TGCTTATGGGCACATGTACCATT | 297 | TGCATCAAATGGAAATGGATTGTTAAATTCA | 298 |
| | i = 15 | | | | |
| HPV58 | SD2-SA5 | TGCTTATGGGCACATGTACCATT | 299 | TGATATTTCCTCCATCGTTTTCCTTGTC | 300 |
| | i = 16 | | | | |
| HPV58 | SD2-SA6 | TGCTTATGGGCACATGTACCATT | 301 | CCCTGTGTACTTTCGTTGTTGGT | 302 |
| | i = 17 | | | | |
| HPV58 | SD2-SA9 | TGCTTATGGGCACATGTACCATT | 303 | GCTTACAACCTTAGACACAGGCA | 304 |
| | i = 18 | | | | |
| HPV59 | SD3-SA4 | AAAGAAGGTTAATAACAGTGCCAGACA | 305 | TCTATTTTTGTCAAATGGCAATTTGTTTGGA | 306 |
| | i = 1 | | | | |
| HPV59 | SD3-SA5 | AAAGAAGGTTAATAACAGTGCCAGACA | 307 | GGTGTCCATCACTGTCTGCAT | 308 |
| | i = 2 | | | | |
| HPV59 | SD3-SA7 | AAAGAAGGTTAATAACAGTGCCAGACA | 309 | CCCAAGTACGTGGCTTCGG | 310 |
| | i = 22 | | | | |
| HPV59 | SD1-SA4 | GCATCAATTGTGTGTTTTGCAAAGG | 311 | TCTATTTTTGTCAAATGGCAATTTGTTTGGA | 312 |
| | i = 4 | | | | |
| HPV59 | SD1-SA5 | GCATCAATTGTGTGTTTTGCAAAGG | 313 | GGTGTCCATCACTGTCTGCAT | 314 |
| | i = 5 | | | | |
| HPV59 | SD1-SA7 | GCATCAATTGTGTGTTTTGCAAAGG | 315 | CCCAAGTACGTGGCTTCGG | 316 |
| | i = 20 | | | | |
| HPV59 | SD1-SA3 | GCATCAATTGTGTGTTTTGCAAAGG | 317 | TGTAAGGCTCGCAATCCGT | 318 |
| | i = 9 | | | | |
| HPV59 | SD5-SA9 | TCCGTTTGCATCCAGGCAA | 319 | TGACATACTCATCAGTGCTGACAAC | 320 |
| | i = 11 | | | | |
| HPV59 | SD5-SA10 | TCCGTTTGCATCCAGGCAA | 321 | GCCAAATTTATTGGGATCAGGTAACTT | 322 |
| | i = 12 | | | | |
| HPV59 | SD2-SA4 | ACTATCCTTTGTGTGTCCTTTGTGT | 323 | TCTATTTTTGTCAAATGGCAATTTGTTTGGA | 324 |
| | i = 15 | | | | |
| HPV59 | SD2-SA5 | ACTATCCTTTGTGTGTCCTTTGTGT | 325 | GGTGTCCATCACTGTCTGCAT | 326 |
| | i = 16 | | | | |
| HPV59 | SD2-SA7 | ACTATCCTTTGTGTGTCCTTTGTGT | 327 | CCCAAGTACGTGGCTTCGG | 328 |
| | i = 21 | | | | |
| HPV59 | SD2-SA9 | ACTATCCTTTGTGTGTCCTTTGTGT | 329 | TGACATACTCATCAGTGCTGACAAC | 330 |
| | i = 18 | | | | |
| HPV59 | SD2-SA10 | ACTATCCTTTGTGTGTCCTTTGTGT | 331 | GCCAAATTTATTGGGATCAGGTAACTT | 332 |
| | i = 19 | | | | |
| HPV66 | SD3-SA4 | GAAGACAGCGGGTATGGCAATA | 333 | CATTACTTAATTCATACACAGGATTACCATT | 334 |
| | i = 1 | | | | |
| HPV66 | SD3-SA5 | GAAGACAGCGGGTATGGCAATA | 335 | TTTTCTTTGTCCTCGTCGTTATCCAA | 336 |
| | i = 2 | | | | |
| HPV66 | SD3-SA6 | GACAGGGAGACAGCTCAACAATTATT | 337 | CTCTCGGTACACAGTTTGCTGATTA | 338 |
| | i = 3 | | | | |
| HPV66 | SD3-SA8 | GAAGACAGCGGGTATGGCAATA | 339 | GGTGGTGGTGGTCCTGTG | 340 |
| | i = 23 | | | | |
| HPV66 | SD1-SA4 | CACCATCTGAGCGAGGTATTACA | 341 | CATTACTTAATTCATACACAGGATTACCATT | 342 |
| | i = 4 | | | | |
| HPV66 | SD1-SA5 | CACCATCTGAGCGAGGTATTACA | 343 | TTTTCTTTGTCCTCGTCGTTATCCAA | 344 |
| | i = 5 | | | | |
| HPV66 | SD1-SA8 | CACCATCTGAGCGAGGTATTACA | 345 | GGTGGTGGTGGTCCTGTG | 346 |
| | i = 24 | | | | |
| HPV66 | SD1-SA1 | CACCATCTGAGCGAGGTATTACA | 347 | GAAATCGTCTTTTATGTTCACAGTGCAA | 348 |
| | i = 7 | | | | |
| HPV66 | SD1-SA2 | CACCATCTGAGCGAGGTATTACA | 349 | AACCTCTTGCAACGTTGGTACT | 350 |
| | i = 8 | | | | |
| HPV66 | SD1-SA3 | CACCATCTGAGCGAGGTATTACA | 351 | TGTACCACACGTAGCTCCTCT | 352 |
| | i = 9 | | | | |
| HPV66 | SD5-SA9 | GTATCAACACACAAAGCCACTGT | 353 | ACAACCTTTGAAACAGGTGTTGGA | 354 |
| | i = 11 | | | | |
| HPV66 | SD2-SA4 | GTTAACAGTAACGTGCCCACTCT | 355 | CATTACTTAATTCVATACACAGGATTACCATT | 356 |
| | i = 15 | | | | |
| HPV66 | SD2-SA5 | GTTAACAGTAACGTGCCCACTCT | 357 | TTTTCTTTGTCCTCGTCGTTATCCAA | 358 |
| | i = 16 | | | | |
| HPV66 | SD2-SA8 | GTTAACAGTAACGTGCCCACTCT | 359 | GGTGGTGGTGGTCCTGTG | 360 |
| | i = 25 | | | | |
| HPV66 | SD2-SA9 | GTTAACAGTAACGTGCCCACTCT | 361 | ACAACCTTTGAAACAGGTGTTGGA | 362 |
| | i = 18 | | | | |
| HPV68 | SD3-SA4 | AGACAACCGGCGTATACAGTG | 363 | CTGTTTTGGTCAAATGGAAATGCATTAG | 364 |
| | i = 1 | | | | |
| HPV68 | SD3-SA6 | AGACAACCGGCGTATACAGTG | 365 | TCGCGGTGGTGTTCTGTAG | 366 |
| | i = 3 | | | | |
| HPV68 | SD1-SA4 | GACATTGGACACTACATTGCATGAC | 367 | CTGTTTTGGTCAAATGGAAATGCATTAG | 368 |
| | i = 4 | | | | |
| HPV68 | SD1-SA1 | GACATTGGACACTACATTGCATGAC | 369 | CTTCGTTTTGTTGTTAGGTGCCTTAG | 370 |
| | i = 7 | | | | |
| HPV68 | SD1-SA6 | GACATTGGACACTACATTGCATGAC | 371 | TCGCGGTGGTGTTCTGTAG | 372 |
| | i = 6 | | | | |
| HPV68 | SD1-SA3 | GACATTGGACACTACATTGCATGAC | 373 | CTGTTGTAGTGTCCGCAGGTT | 374 |
| | i = 9 | | | | |
| HPV68 | SD5-SA9 | AGTAGAAGTGCAGGCCAAAACAA | 375 | ATTGACAACCTTCGCCACTGA | 376 |
| | i = 11 | | | | |
| HPV68 | SD2-SA4 | TCCGTGGTGTGCAACTGAA | 377 | CTGTTTTGGTCAAATGGAAATGCATTAG | 378 |
| | i = 15 | | | | |
| HPV68 | SD2-SA6 | TCCGTGGTGTGCAACTGAA | 379 | TCGCGGTGGTGTTCTGTAG | 380 |
| | i = 17 | | | | |
| HPV68 | SD2-SA9 | TCCGTGGTGTGCAACTGAA | 381 | ATTGACAACCTTCGCCACTGA | 382 |
| | i = 1 | | | | |
| HPV73 | SD3-SA4 | AAACGAAGACTGTTTGAGGAGCA | 383 | GGGTTCCCATTACTGTCAAATGGA | 384 |
| | i = 1 | | | | |
| HPV73 | SD3-SA6 | AAACGAAGACTGTTTGAGGAGCA | 385 | TGGTGTTGGTGGTTGTGGT | 386 |
| | i = 3 | | | | |
| HPV73 | SD1-SA4 | AGCGTTATGTGACGAAGTGAATATTTCT | 387 | GGGTTCCCATTACTGTCAAATGGA | 388 |
| | i = 4 | | | | |
| HPV73 | SD1-SA6 | AGCGTTATGTGACGAAGTGAATATTTCT | 389 | TGGTGTTGGTGGTTGTGGT | 390 |
| | i = 6 | | | | |
| HPV73 | SD1-SA1 | AGCGTTATGTGACGAAGTGAATATTTCT | 391 | CTGTTCTGCTATTTGATGAAACCGTTTT | 392 |
| | i = 7 | | | | |
| HPV73 | SD1-SA2 | AGCGTTATGTGACGAAGTGAATATTTCT | 393 | TTCGGTTGTTGGTTTCAGGTCTAA | 394 |
| | i = 8 | | | | |
| HPV73 | SD1-SA3 | AGCGTTATGTGACGAAGTGAATATTTCT | 395 | CCTAGTGTACCCATAAGCAACTCTTCTA | 396 |
| | i = 9 | | | | |
| HPV73 | SD5-SA9 | ACCTACATCCCACCACAGAGT | 397 | GCTTACAACCTTAGACACAGACACA | 398 |
| | i = 11 | | | | |
| HPV73 | SD5-SA10 | ACCTACATCCCACCACAGAGT | 399 | ACGAAGCCTAAACACCCTGTATTG | 400 |
| | i = 12 | | | | |
| HPV73 | SD2-SA4 | TGCTTATGGGTACACTAGGTATTGTGT | 401 | GGGTTCCCATTACTGTCAAATGGA | 402 |
| | i = 15 | | | | |
| HPV73 | SD2-SA6 | TGCTTATGGGTACACTAGGTATTGTGT | 403 | TGGTGTTGGTGGTTGTGGT | 404 |
| | i = 17 | | | | |
| HPV73 | SD2-SA9 | TGCTTATGGGTACACTAGGTATTGTGT | 405 | GCTTACAACCTTAGACACAGACACA | 406 |
| | i = 18 | | | | |
| HPV73 | SD2-SA10 | TGCTTATGGGTACACTAGGTATTGTGT | 407 | ACGAAGCCTAAACACCCTGTATTG | 408 |
| | i = 19 | | | | |
| HPV82 | SD3-SA4 | CCGGACAGTGGATATGGCAATA | 409 | CATCATTTAGTGCATATACAGGATTC | 410 |
| | i = 1 | | | | |
| HPV82 | SD3-SA6 | CCGGACAGTGGATATGGCAATA | 411 | GGGTGTTCGATAGCTGTTCAA | 412 |
| | i = 3 | | | | |
| HPV82 | SD1-SA4 | CCTGCAATACGTCTATGCACAAT | 413 | CATCATTTAGTGCATATACAGGATTCCC | 414 |
| | i = 4 | | | | |
| HPV82 | SD1-SA6 | CCTGCAATACGTCTATGCACAAT | 415 | GGGTGTTCGATAGCTGTTCAA | 416 |
| | i = 6 | | | | |
| HPV82 | SD1-SA1 | CCTGCAATACGTCTATGCACAAT | 417 | TTTTTTGTCGTCCACCACCTTTTG | 418 |
| | i = 7 | | | | |
| HPV82 | SD1-SA2 | CCTGCAATACGTCTATGCACAAT | 419 | TCCAACACTATGTCCTTTAATTGTGGT | 420 |
| | i = 8 | | | | |
| HPV82 | SD10SA3 | CCTGCAATACGTCTATGCACAAT | 421 | CCAGTAACATTTGCTGAAATATGCGAA | 422 |
| | i = 9 | | | | |
| HPV82 | SD5-SA9 | TGCGACCACCAAATACACTGT | 423 | GTGTTGACAATGCGTGACACT | 424 |
| | i = 1 | | | | |
| HPV82 | SD2-SA4 | CGTGGTGTGCGACCAACTAA | 425 | CATCATTTAGTGCATATACAGGATTCCC | 426 |
| | i = 15 | | | | |
| HPV82 | SD2-SA6 | CGTGGTGTGCGACCAACTAA | 427 | GGGTGTTCGATAGCTGTTCAA | 428 |
| | i = 17 | | | | |
| HPV82 | SD2-SA9 | CGTGGTGTGCGACCAACTAA | 429 | GTGTTGACAATGCGTGACACT | 430 |
| | i = 18 | | | | |

TABLE 2Abis

| | | Forward | Reverse | | |
|---|---|---|---|---|---|
| | Splice | primer | primer | | |
| | junction | SEQ ID | SEQ ID | Amplicon | Amplicon |
| HPV type | i = | NO | NO | nucleic acid sequence | SEQ ID NO |
| HPV16 | SD3-SA4 | 1 | 2 | GGAAACTCAGCAGATGTTAC | 1501 |
| | i = 1 | | | AGATTCTAGGTGGCCTTATT | |
| | | | | TACATAATAGATTGGTGGTG | |
| | | | | TTTACATT | |
| HPV16 | SD3-SA5 | 3 | 4 | GGAAACTCAGCAGATGTTAC | 1502 |
| | i = 2 | | | AGGACGTGGTCCAGATTAAG | |
| | | | | TTTGCACGAGGACGAGGACA | |
| | | | | AGGAAAACGATGGAGACT | |
| HPV16 | SD3-SA6 | 5 | 6 | GGAAACTCAGCAGATGTTAC | 1503 |
| | i = 3 | | | AGCAGCAACGAAGTATCCTC | |
| | | | | TCCTGAAATTATTAGGCAGC | |
| | | | | ACTTGGCCAACCACCCCGCC | |
| | | | | GCGACCC | |
| HPV16 | SD1-SA4 | 7 | 8 | GATATAATATTAGAATGTGT | 1504 |
| | i = 4 | | | GTACTGCAAGCAACAGTTAC | |
| | | | | TGCGACGTGAGATTCTAGGT | |
| | | | | GGCCTTATTTACATAATAGA | |
| | | | | TTGGTGGTGTTTACATT | |
| HPV16 | SD1-SA5 | 9 | 10 | GATATAATATTAGAATGTGT | 1505 |
| | i = 5 | | | GTACTGCAAGCAACAGTTAC | |
| | | | | TGCGACGTGAGGACGTGGTC | |
| | | | | CAGATTAAGTTTGCACGAGG | |
| | | | | ACGAGGACAAGGAAAACGAT | |
| | | | | GGAGACT | |
| HPV16 | SD1-SA6 | 11 | 12 | GATATAATATTAGAATGTGT | 1506 |
| | i = 6 | | | GTACTGCAAGCAACAGTTAC | |
| | | | | TGCGACGTGAGCAGCAACGA | |
| | | | | AGTATCCTCTCCTGAAATTA | |
| | | | | TTAGGCAGCACTTGGCCAAC | |
| | | | | CACCCCGCCGCGACCC | |
| HPV16 | SD1-SA1 | 13 | 14 | GATATAATATTAGAATGTGT | 1507 |
| | i = 7 | | | GTACTGCAAGCAACAGTTAC | |
| | | | | TGCGACGTGAGGTGTATTAA | |
| | | | | CTGTCAAAAGCCACTGTGTC | |
| | | | | C | |
| HPV16 | SD1-SA2 | 15 | 16 | GATATAATATTAGAATGTGT | 1508 |
| | i = 8 | | | GTACTGCAAGCAACAGTTAC | |
| | | | | TGCGACGTGAGATCATCAAG | |
| | | | | AACACGTAGAGAAACCCAGC | |
| | | | | TGTAATCATGCATGGAGATA | |
| | | | | CACCTACATTGCATGAATAT | |
| | | | | ATGTT | |
| HPV16 | SD1-SA3 | 17 | 18 | GATATAATATTAGAATGTGT | 1509 |
| | i = 9 | | | GTACTGCAAGCAACAGTTAC | |
| | | | | TGCGACGTGAGTGTGACTCT | |
| | | | | ACGCTTCGGTTGTGCGTACA | |
| | | | | AAGCACACACGTAGACAT | |
| HPV16 | SD5-SA9 | 19 | 20 | TGTAATAGTAACACTACACC | 1510 |
| | i = 11 | | | CATAGTACATTTAAAAGATG | |
| | | | | TCTCTTTGGCTGCCTAGTGA | |
| | | | | GGCCACTGTCTACTTGCCTC | |
| | | | | CTGTCC | |
| HPV16 | SD2-SA4 | 21 | 22 | TCTCAGAAACCATAATCTAC | 1511 |
| | i = 15 | | | CATGGCTGATCCTGCAGATT | |
| | | | | CTAGGTGGCCTTATTTACAT | |
| | | | | AATAGATTGGTGGTGTTTAC | |
| | | | | ATT | |
| HPV16 | SD2-SA5 | 23 | 24 | TCTCAGAAACCATAATCTAC | 1512 |
| | i = 16 | | | CATGGCTGATCCTGCAGGAC | |
| | | | | GTGGTCCAGATTAAGTTTGC | |
| | | | | ACGAGGACGAGGACAAGGAA | |
| | | | | AACGATGGAGACT | |
| HPV16 | SD2-SA6 | 25 | 26 | TCTCAGAAACCATAATCTAC | 1513 |
| | i = 17 | | | CATGGCTGATCCTGCAGCAG | |
| | | | | CAACGAAGTATCCTCTCCTG | |
| | | | | AAATTATTAGGCAGCACTTG | |
| | | | | GCCAACCACCCCGCCGCGAC | |
| | | | | CC | |
| HPV16 | SD2-SA9 | 27 | 28 | TCTCAGAAACCATAATCTAC | 1514 |
| | i = 18 | | | CATGGCTGATCCTGCAGATG | |
| | | | | TCTCTTTGGCTGCCTAGTGA | |
| | | | | GGCCACTGTCTACTTGCCTC | |
| | | | | CTGTCC | |
| HPV18 | SD3-SA4 | 29 | 30 | GAAGTGGAAGCAACACAGAT | 1515 |
| | i = 1 | | | TCAGGATAATAGATGGCCAT | |
| | | | | ATTTAGAAAGTAGAATAACA | |
| | | | | GTATTTGAATTTCCAAATGC | |
| | | | | ATTTCCATTTGATAAAAA | |
| HPV18 | SD3-SA5 | 31 | 32 | GAAGTGGAAGCAACACAGAT | 1516 |
| | i = 2 | | | TCAGGACATGGTCCAGATTA | |
| | | | | GATTTGCACGAGGAAGAGGA | |
| | | | | AG | |
| HPV18 | SD3-SA6 | 33 | 34 | GAAGTGGAAGCAACACAGAT | 1517 |
| | i = 3 | | | TCAGCTTGTTAAACAGCTAC | |
| | | | | AGCACACCCCCTCACCGTAT | |
| | | | | TCCAGCACCGTGTCCGTGGG | |
| | | | | CACCGCAA | |
| HPV18 | SD1-SA4 | 35 | 36 | GTATATTGCAAGACAGTATT | 1518 |
| | i = 4 | | | GGAACTTACAGAGGATAATA | |
| | | | | GATGGCCATATTTAGAAAGT | |
| | | | | AGAATAACAGTATTTGAATT | |
| | | | | TCCAAATGCATTTCCATTTG | |
| | | | | ATAAAAA | |
| HPV18 | SD1-SA5 | 37 | 38 | GTATATTGCAAGACAGTATT | 1519 |
| | i = 5 | | | GGAACTTACAGAGGACATGG | |
| | | | | TCCAGATTAGATTTGCACGA | |
| | | | | GGAAGAGGAAG | |
| HPV18 | SD1-SA6 | 39 | 40 | GTATATTGCAAGACAGTATT | 1520 |
| | i = 6 | | | GGAACTTACAGAGCTTGTTA | |
| | | | | AACAGCTACAGCACACCCCC | |
| | | | | TCACCGTATTCCAGCACCGT | |
| | | | | GTCCGTGGGCACCGCAA | |
| HPV18 | SD1-SA1 | 41 | 42 | GTATATTGCAAGACAGTATT | 1521 |
| | i = 7 | | | GGAACTTACAGAGGTGCCTG | |
| | | | | CGGTGCCAGAAACCGTTGAA | |
| | | | | TCCAGCAGAAAAACTTAGAC | |
| | | | | ACCTTAATGAAAAACG | |
| HPV18 | SD1-SA3 | 43 | 44 | GTATATTGCAAGACAGTATT | 1522 |
| | i = 9 | | | GGAACTTACAGAGTGTGAAG | |
| | | | | CCAGAATTGAGCTAGTAGTA | |
| | | | | GAAAGCTCAGCAGACGACCT | |
| | | | | TCG | |
| HPV18 | SD4-SA6 | 45 | 46 | TGCGAGGAACTATGGAATAC | 1523 |
| | i = 10 | | | AGAACCTACTCACTGCTTTA | |
| | | | | AAAAAGCTTGTTAAACAGCT | |
| | | | | ACAGCACACCCCCTCACCGT | |
| | | | | ATTCCAGCACCGTGTCCGTG | |
| | | | | GGCACCGCAA | |
| HPV18 | SD5-SA9 | 47 | 48 | CAAAAGACGGAAACTCTGTA | 1524 |
| | i = 11 | | | GTGGTAACACTACGCCTATA | |
| | | | | ATACATTTAAAAGATGGCTT | |
| | | | | TGTGGCGGCCTAGTGACAAT | |
| | | | | ACCGTATATCTTCCACC | |
| HPV18 | SD5-SA10 | 49 | 50 | CAAAAGACGGAAACTCTGTA | 1525 |
| | i = 12 | | | GTGGTAACACTACGCCTATA | |
| | | | | ATACATTTAAAAGGTGGTGG | |
| | | | | CAATAAGCAGGATATTCCTA | |
| | | | | AGGTTTCTGCATACCAAT | |
| HPV18 | SD6-SA9 | 51 | 52 | ATATCATCCACCTGGCATTG | 1526 |
| | i = 13 | | | GACAGATGGCTTTGTGGCGG | |
| | | | | CCTAGTGACAATACCGTATA | |
| | | | | TCTTCCACC | |
| HPV18 | SD6-SA10 | 53 | 54 | ATATCATCCACCTGGCATTG | 1527 |
| | i = 14 | | | GACAGGTGGTGGCAATAAGC | |
| | | | | AGGATATTCCTAAGGTTTCT | |
| | | | | GCATACCAAT | |
| HPV18 | SD2-SA4 | 55 | 56 | CAATGGCTGATCCAGAAGGA | 1528 |
| | i = 15 | | | TAATAGATGGCCATATTTAG | |
| | | | | AAAGTAGAATAACAGTATTT | |
| | | | | GAATTTCCAAATGCATTTCC | |
| | | | | ATTTGATAAAAA | |
| HPV18 | SD2-SA5 | 57 | 58 | CAATGGCTGATCCAGAAGGA | 1529 |
| | i = 16 | | | CATGGTCCAGATTAGATTTG | |
| | | | | CACGAGGAAGAGGAAG | |
| HPV18 | SD2-SA8 | 59 | 60 | CAATGGCTGATCCAGAAGCT | 1530 |
| | i = 25 | | | TGTTAAACAGCTACAGCACA | |
| | | | | CCCCCTCACCGTATTCCAGC | |
| | | | | ACCGTGTCCGTGGGCACCGC | |
| | | | | AA | |
| HPV18 | SD2-SA9 | 61 | 62 | CAATGGCTGATCCAGAAGAT | 1531 |
| | i = 18 | | | GGCTTTGTGGCGGCCTAGTG | |
| | | | | ACAATACCGTATATCTTCCA | |
| | | | | CC | |
| HPV18 | SD2-SA10 | 63 | 64 | CAATGGCTGATCCAGAAGGT | 1532 |
| | i = 19 | | | GGTGGCAATAAGCAGGATAT | |
| | | | | TCCTAAGGTTTCTGCATACC | |
| | | | | AAT | |
| HPV31 | SD3-SA4 | 65 | 66 | GGAAACGCAGCAGATGGTAC | 1533 |
| | i = 1 | | | AGGATGACAGATGGCCATAC | |
| | | | | C | |
| HPV31 | SD3-SA5 | 67 | 68 | GGAAACGCAGCAGATGGTAC | 1534 |
| | i = 2 | | | AGGACGTGGTGCAGATTAAA | |
| | | | | TTTGCACGAGGAAGAGGACA | |
| | | | | AAG | |
| HPV31 | SD3-SA6 | 69 | 70 | GGAAACGCAGCAGATGGTAC | 1535 |
| | i = 3 | | | AGCAGTGACGAAATATCCTT | |
| | | | | TGCTGGGATTGTTACAAAGC | |
| | | | | TACCAACAGC | |
| HPV31 | SD1-SA4 | 71 | 72 | GAACTAAGATTGAATTGTGT | 1536 |
| | i = 4 | | | CTACTGCAAAGGTCAGTTAA | |
| | | | | CAGAAACAGAGGATGACAGA | |
| | | | | TGGCCATACC | |
| HPV31 | SD1-SA5 | 73 | 74 | GAACTAAGATTGAATTGTGT | 1537 |
| | i = 5 | | | CTACTGCAAAGGTCAGTTAA | |
| | | | | CAGAAACAGAGGACGTGGTG | |
| | | | | CAGATTAAATTTGCACGAGG | |
| | | | | AAGAGGACAAAG | |
| HPV31 | SD1-SA6 | 75 | 76 | GAACTAAGATTGAATTGTGT | 1538 |
| | i = 6 | | | CTACTGCAAAGGTCAGTTAA | |
| | | | | CAGAAACAGAGCAGTGACGA | |
| | | | | AATATCCTTTGCTGGGATTG | |
| | | | | TTACAAAGCTACCAACAGC | |
| HPV31 | SD1-SA1 | 77 | 78 | GAACTAAGATTGAATTGTGT | 1539 |
| | i = 7 | | | CTACTGCAAAGGTCAGTTAA | |
| | | | | CAGAAACAGAGGTGTATAAC | |
| | | | | GTGTCA | |
| HPV31 | SD1-SA2 | 79 | 80 | GAACTAAGATTGAATTGTGT | 1540 |
| | i = 8 | | | CTACTGCAAAGGTCAGTTAA | |
| | | | | CAGAAACAGAGAAGACCTCG | |
| | | | | TACTGAAACCCAAGTGTAAA | |
| | | | | CATGCGTGGAG | |
| HPV31 | SD1-SA3 | 81 | 82 | GAACTAAGATTGAATTGTGT | 1541 |
| | i = 9 | | | CTACTGCAAAGGTCAGTTAA | |
| | | | | CAGAAACAGAGTGTAAGTCT | |
| | | | | ACACTTCGTTTGTGTGTACA | |
| | | | | GAGCACACAAGTAGA | |
| HPV31 | SD5-SA9 | 83 | 84 | AACAAGGGCTGTCAGTTGTC | 1542 |
| | i = 11 | | | CTGCAACTACACCTATAATA | |
| | | | | CACTTAAAAGATGTCTCTGT | |
| | | | | GGCGGCCTAGCGAGGCTACT | |
| | | | | GTCTACT | |
| HPV31 | SD2-SA4 | 85 | 86 | TCTACTAGACTGTAACTACA | 1543 |
| | i = 15 | | | ATGGCTGATCCAGCAGGATG | |
| | | | | ACAGATGGCCATACC | |
| HPV31 | SD2-SA5 | 87 | 88 | TCTACTAGACTGTAACTACA | 1544 |
| | i = 16 | | | ATGGCTGATCCAGCAGGACG | |
| | | | | TGGTGCAGATTAAATTTGCA | |
| | | | | CGAGGAAGAGGACAAAG | |
| HPV31 | SD2-SA6 | 89 | 90 | TCTACTAGACTGTAACTACA | 1545 |
| | i = 17 | | | ATGGCTGATCCAGCAGCAGT | |
| | | | | GACGAAATATCCTTTGCTGG | |
| | | | | GATTGTTACAAAGCTACCAA | |
| | | | | CAGC | |
| HPV31 | SD2-SA9 | 91 | 92 | TCTACTAGACTGTAACTACA | 1546 |
| | i = 18 | | | ATGGCTGATCCAGCAGATGT | |
| | | | | CTCTGTGGCGGCCTAGCGAG | |
| | | | | GCTACTGTCTACT | |
| HPV33 | SD3-SA4 | 93 | 94 | GCAATACTGAAGTGGAAACT | 1547 |
| | i = 1 | | | CAGCAGATGGTACAACAGAC | |
| | | | | TCTAGATGGCCATATTTACA | |
| | | | | TAGTAGATTAACAGTATTTG | |
| | | | | AATTTAAAAATCCATTCCCA | |
| HPV33 | SD3-SA5 | 95 | 96 | GCAATACTGAAGTGGAAACT | 1548 |
| | i = 2 | | | CAGCAGATGGTACAACAGGA | |
| | | | | CGTGGTGCAAATTAGATTTA | |
| | | | | ATAGAGGAAGAG | |
| HPV33 | SD3-SA6 | 97 | 98 | GCAATACTGAAGTGGAAACT | 1549 |
| | i = 3 | | | CAGCAGATGGTACAACAGCA | |
| | | | | ACCAAATATCCACTACTGAA | |
| | | | | ACTGCTGACATACAGACAG | |
| HPV33 | SD1-SA4 | 99 | 100 | GAACTACAGTGCGTGGAATG | 1550 |
| | i = 4 | | | CAAAAAACCTTTGCAACGAT | |
| | | | | CTGAGACTCTAGATGGCCAT | |
| | | | | ATTTACATAGTAGATTAACA | |
| | | | | GTATTTGAATTTAAAAATCC | |
| | | | | ATTCCCA | |
| HPV33 | SD1-SA5 | 101 | 102 | GAACTACAGTGCGTGGAATG | 1551 |
| | i = 5 | | | CAAAAAACCTTTGCAACGAT | |
| | | | | CTGAGGACGTGGTGCAAATT | |
| | | | | AGATTTAATAGAGGAAGAG | |
| HPV33 | SD1-SA6 | 103 | 104 | GAACTACAGTGCGTGGAATG | 1552 |
| | i = 6 | | | CAAAAAACCTTTGCAACGAT | |
| | | | | CTGAGCAACCAAATATCCAC | |
| | | | | TACTGAAACTGCTGACATAC | |
| | | | | AGACAG | |
| HPV33 | SD1-SA1 | 105 | 106 | GAACTACAGTGCGTGGAATG | 1553 |
| | i = 7 | | | CAAAAAACCTTTGCAACGAT | |
| | | | | CTGAGGTGTATTATATGTCA | |
| | | | | AAGACCTTTGTGTCCTCAAG | |
| | | | | AAAA | |
| HPV33 | SD1-SA2 | 107 | 108 | GAACTACAGTGCGTGGAATG | 1554 |
| | i = 8 | | | CAAAAAACCTTTGCAACGAT | |
| | | | | CTGAGGTCCCGACGTAGAGA | |
| | | | | AACTGCACTGTGACGTGTAA | |
| | | | | AAACGCCATGAGAGG | |
| HPV33 | SD5-SA9 | 109 | 110 | ACAAGCAGCGGACTGTGTGT | 1555 |
| | i = 11 | | | AGTTCTAACGTTGCACCTAT | |
| | | | | AGTGCATTTAAAAGATGTCC | |
| | | | | GTGTGGCGGCCTAGTGAGGC | |
| | | | | CACAGTGTACCTGCCTCCTG | |
| | | | | TA | |
| HPV33 | SD2-SA4 | 111 | 112 | CAATAAACATCATCTACAAT | 1556 |
| | i = 15 | | | GGCCGATCCTGAAGACTCTA | |
| | | | | GATGGCCATATTTACATAGT | |
| | | | | AGATTAACAGTATTTGAATT | |
| | | | | TAAAAATCCATTCCCA | |
| HPV33 | SD2-SA5 | 113 | 114 | CAATAAACATCATCTACAAT | 1557 |
| | i = 16 | | | GGCCGATCCTGAAGGACGTG | |
| | | | | GTGCAAATTAGATTTAATAG | |
| | | | | AGGAAGAG | |
| HPV33 | SD2-SA6 | 115 | 116 | CAATAAACATCATCTACAAT | 1558 |
| | i = 17 | | | GGCCGATCCTGAAGCAACCA | |
| | | | | AATATCCACTACTGAAACTG | |
| | | | | CTGACATACAGACAG | |
| HPV33 | SD2-SA9 | 117 | 118 | CAATAAACATCATCTACAAT | 1559 |
| | i = 18 | | | GGCCGATCCTGAAGATGTCC | |
| | | | | GTGTGGCGGCCTAGTGAGGC | |
| | | | | CACAGTGTACCTGCCTCCTG | |
| | | | | TA | |
| HPV35 | SD3-SA4 | 119 | 120 | ATGGCAATTCTGAAGTGGAA | 1560 |
| | i = 1 | | | ATACAGCAGATACAACAGAT | |
| | | | | GACAGGTGGCCATACTTACA | |
| | | | | TAGCA | |
| HPV35 | SD3-SA5 | 121 | 122 | ATGGCAATTCTGAAGTGGAA | 1561 |
| | i = 2 | | | ATACAGCAGATACAACAGGA | |
| | | | | CGTGGTGCAGATTAAATTTG | |
| | | | | CACGAGGAAGAGGA | |
| HPV35 | SD3-SA6 | 123 | 124 | ATGGCAATTCTGAAGTGGAA | 1562 |
| | i = 3 | | | ATACAGCAGATACAACAGCA | |
| | | | | GCACAGAACTATCCACTGCT | |
| | | | | GAAATTGCTACACAGCTACA | |
| | | | | CGCCTACAACACC | |
| HPV35 | SD1-SA4 | 125 | 126 | TTGTTTGAATTGTGTATACT | 1563 |
| | i = 4 | | | GCAAACAAGAATTACAGCGG | |
| | | | | AGTGAGATGACAGGTGGCCA | |
| | | | | TACTTACATAGCA | |
| HPV35 | SD1-SA5 | 127 | 128 | TTGTTTGAATTGTGTATACT | 1564 |
| | i = 5 | | | GCAAACAAGAATTACAGCGG | |
| | | | | AGTGAGGACGTGGTGCAGAT | |
| | | | | TAAATTTGCACGAGGAAGAG | |
| | | | | GA | |
| HPV35 | SD1-SA6 | 129 | 130 | TTGTTTGAATTGTGTATACT | 1565 |
| | i = 6 | | | GCAAACAAGAATTACAGCGG | |
| | | | | AGTGAGCAGCACAGAACTAT | |
| | | | | CCACTGCTGAAATTGCTACA | |
| | | | | CAGCTACACGCCTACAACAC | |
| | | | | C | |
| HPV35 | SD1-SA1 | 131 | 132 | TTGTTTGAATTGTGTATACT | 1566 |
| | i = 7 | | | GCAAACAAGAATTACAGCGG | |
| | | | | AGTGAGGTGTATTACATGTC | |
| | | | | AAAAACCGCTGTGTCCAGTT | |
| | | | | GAAAAGCAAAGACATTTAGA | |
| | | | | AGAAAA | |
| HPV35 | SD5-SA9 | 133 | 134 | CCGGTGTGGTAGTTGTAGTA | 1567 |
| | i = 11 | | | CAACTACACCTATAGTACAT | |
| | | | | TTAAAAGATGTCTCTGTGGC | |
| | | | | GGTCTAACGAAGCCACTGTC | |
| | | | | TACCTGCCTCCAGTGTC | |
| HPV35 | SD5-SA10 | 135 | 136 | CCGGTGTGGTAGTTGTAGTA | 1568 |
| | i = 12 | | | CAACTACACCTATAGTACAT | |
| | | | | TTAAAAGATTCTAATAAAAT | |
| | | | | AGCAGTACC | |
| HPV35 | SD2-SA4 | 137 | 138 | CTACAATGGCTGATCCTGCA | 1569 |
| | i = 15 | | | GATGACAGGTGGCCATACTT | |
| | | | | ACATAGCA | |
| HPV35 | SD2-SA5 | 139 | 140 | CTACAATGGCTGATCCTGCA | 1570 |
| | i = 16 | | | GGACGTGGTGCAGATTAAAT | |
| | | | | TTGCACGAGGAAGAGGA | |
| HPV35 | SD2-SA6 | 141 | 142 | CTACAATGGCTGATCCTGCA | 1571 |
| | i = 17 | | | GCAGCACAGAACTATCCACT | |
| | | | | GCTGAAATTGCTACACAGCT | |
| | | | | ACACGCCTACAACACC | |
| HPV35 | SD2-SA9 | 143 | 144 | CTACAATGGCTGATCCTGCA | 1572 |
| | i = 18 | | | GATGTCTCTGTGGCGGTCTA | |
| | | | | ACGAAGCCACTGTCTACCTG | |
| | | | | CCTCCAGTGTC | |
| HPV35 | SD2-SA10 | 145 | 146 | CTACAATGGCTGATCCTGCA | 1573 |
| | i = 19 | | | GATTCTAATAAAATAGCAGT | |
| | | | | ACC | |
| HPV39 | SD3-SA4 | 147 | 148 | GCGGATATGGCAATATGGAA | 1574 |
| | i = 1 | | | GTGGAAACAGCTGAAGTGGA | |
| | | | | GGAGACGATAGGTGGCCATA | |
| | | | | TTTACGTAGTAGGCTAACAG | |
| | | | | TGTTTAAATTTC | |
| HPV39 | SD3-SA7 | 149 | 150 | GCGGATATGGCAATATGGAA | 1575 |
| | i = 22 | | | GTGGAAACAGCTGAAGTGGA | |
| | | | | GGAGTGACGGATCGGTACCC | |
| | | | | ACTACTGAACTTACTACCGA | |
| | | | | A | |
| HPV39 | SD1-SA4 | 151 | 152 | GCCTGTGTCTATTGCAGACG | 1576 |
| | i = 4 | | | ACCACTACAGCAAACCGAGA | |
| | | | | CGATAGGTGGCCATATTTAC | |
| | | | | GTAGTAGGCTAACAGTGTTT | |
| | | | | AAATTTC | |
| HPV39 | SD1-SA7 | 153 | 154 | GCCTGTGTCTATTGCAGACG | 1577 |
| | i = 20 | | | ACCACTACAGCAAACCGAGT | |
| | | | | GACGGATCGGTACCCACTAC | |
| | | | | TGAACTTACTACCGAA | |
| HPV39 | SD1-SA1 | 155 | 156 | GCCTGTGTCTATTGCAGACG | 1578 |
| | i = 7 | | | ACCACTACAGCAAACCGAGG | |
| | | | | TGCATGTGTTGTCTGAAACC | |
| | | | | GCTGTGTCCAGCAGAAAAAT | |
| | | | | TAAGACACCTAAATAGCAAA | |
| | | | | CGAAGATTTCAT | |
| HPV39 | SD1-SA3 | 157 | 158 | GCCTGTGTCTATTGCAGACG | 1579 |
| | i = 9 | | | ACCACTACAGCAAACCGAGT | |
| | | | | GTAACAACACACTGCAGCTG | |
| | | | | GTAGTAGAAGCCTCACGG | |
| HPV39 | SD5-SA9 | 159 | 160 | ACACAAGACGGTACCTCAGT | 1580 |
| | i = 11 | | | TGTGGTAACACTACGCCTAT | |
| | | | | AATACATTTAAAAGATGGCT | |
| | | | | ATGTGGCGGTCTAGTGACAG | |
| | | | | CATGGTGTATTTGCCTCCAC | |
| | | | | CTTC | |
| HPV39 | SD2-SA4 | 161 | 162 | ACCAGTAACCTGCTATGGCC | 1581 |
| | i = 15 | | | AATCGTGAAGACGATAGGTG | |
| | | | | GCCATATTTACGTAGTAGGC | |
| | | | | TAACAGTGTTTAAATTTC | |
| HPV39 | SD2-SA7 | 163 | 164 | ACCAGTAACCTGCTATGGCC | 1582 |
| | i = 21 | | | AATCGTGAAGTGACGGATCG | |
| | | | | GTACCCACTACTGAACTTAC | |
| | | | | TACCGAA | |
| HPV39 | SD2-SA9 | 165 | 166 | ACCAGTAACCTGCTATGGCC | 1583 |
| | i = 18 | | | AATCGTGAAGATGGCTATGT | |
| | | | | GGCGGTCTAGTGACAGCATG | |
| | | | | GTGTATTTGCCTCCACCTTC | |
| HPV45 | SD3-SA4 | 167 | 168 | GAAGTGGAAGCTGCAGAGAC | 1584 |
| | i = 1 | | | TCAGATAATAAATGGCCATA | |
| | | | | TTTAGAAAGTAGGGTG | |
| HPV45 | SD3-SA5 | 169 | 170 | GAAGTGGAAGCTGCAGAGAC | 1585 |
| | i = 2 | | | TCAGGACATGGTCCAGATTA | |
| | | | | GATTTGCACGAGGACGATGA | |
| | | | | AG | |
| HPV45 | SD3-SA8 | 171 | 172 | GAAGTGGAAGCTGCAGAGAC | 1586 |
| | i = 23 | | | TCAGATTGTTAGACAGCTAC | |
| | | | | AACACGCCTCCACGTCGACC | |
| | | | | CC | |
| HPV45 | SD1-SA4 | 173 | 174 | ATATTGCAAAGCAACATTGG | 1587 |
| | i = 4 | | | AACGCACAGAGATAATAAAT | |
| | | | | GGCCATATTTAGAAAGTAGG | |
| | | | | GTG | |
| HPV45 | SD1-SA5 | 175 | 176 | ATATTGCAAAGCAACATTGG | 1588 |
| | i = 5 | | | AACGCACAGAGGACATGGTC | |
| | | | | CAGATTAGATTTGCACGAGG | |
| | | | | ACGATGAAG | |
| HPV45 | SD1-SA8 | 177 | 178 | ATATTGCAAAGCAACATTGG | 1589 |
| | i = 24 | | | AACGCACAGAGATTGTTAGA | |
| | | | | CAGCTACAACACGCCTCCAC | |
| | | | | GTCGACCCC | |
| HPV45 | SD1-SA1 | 179 | 180 | ATATTGCAAAGCAACATTGG | 1590 |
| | i = 7 | | | AACGCACAGAGGTGCCTGCG | |
| | | | | GTGCCAGAAACCATTGAACC | |
| | | | | CAGCAGA | |
| HPV45 | SD1-SA3 | 181 | 182 | ATATTGCAAAGCAACATTGG | 1591 |
| | i = 9 | | | AACGCACAGAGTGTGACGGC | |
| | | | | AGAATTGAGCTTACAGTAGA | |
| | | | | GAGCTCGGCAGAGGACCTT | |
| HPV45 | SD5-SA9 | 183 | 184 | AAGAAGGAAAGTGTGTAGTG | 1592 |
| | i = 11 | | | GTAACACTACGCCTATAATA | |
| | | | | CACTTAAAAGATGGCTTTGT | |
| | | | | GGCGGCCTAGTGACAGTACG | |
| | | | | GTATATCTTCCACCACCTTC | |
| HPV45 | SD6-SA9 | 185 | 186 | ATATCCTCCACCTGGCATTG | 1593 |
| | i = 13 | | | GACAGATGGCTTTGTGGCGG | |
| | | | | CCTAGTGACAGTACGGTATA | |
| | | | | TCTTCCACCACCTTC | |
| HPV45 | SD2-SA4 | 187 | 188 | CCGTGGTGTGCAACTAACCA | 1594 |
| | i = 15 | | | ATAATCTACAATGGCGGATC | |
| | | | | CAGAAGATAATAAATGGCCA | |
| | | | | TATTTAGAAAGTAGGGTG | |
| HPV45 | SD2-SA5 | 189 | 190 | CCGTGGTGTGCAACTAACCA | 1595 |
| | i = 16 | | | ATAATCTACAATGGCGGATC | |
| | | | | CAGAAGGACATGGTCCAGAT | |
| | | | | TAGATTTGCACGAGGACGAT | |
| | | | | GAAG | |
| HPV45 | SD2-SA8 | 191 | 192 | CCGTGGTGTGCAACTAACCA | 1596 |
| | i = 25 | | | ATAATCTACAATGGCGGATC | |
| | | | | CAGAAGATTCiTTAGACAGC | |
| | | | | TACAACACGCCTCCACGTCG | |
| | | | | ACCCC | |
| HPV45 | SD2-SA9 | 193 | 194 | CCGTGGTGTGCAACTAACCA | 1597 |
| | i = 18 | | | ATAATCTACAATGGCGGATC | |
| | | | | CAGAAGATGGCTTTGTGGCG | |
| | | | | GCCTAGTGACAGTACGGTAT | |
| | | | | ATCTTCCACCACCTTC | |
| HPV51 | SD3-SA4 | 195 | 196 | CACAAGTGGAAACTGTGGAA | 1598 |
| | i = 1 | | | GCAACGTTGCAGGATGCAAA | |
| | | | | CCTAATGTATTTACATACAA | |
| | | | | GGGTAACAGTATTAAAGTTT | |
| | | | | TTAAATACATTTCCATTTGA | |
| | | | | TAACA | |
| HPV51 | SD3-SA6 | 197 | 198 | CACAAGTGGAAACTGTGGAA | 1599 |
| | i = 3 | | | GCAACGTTGCAGTACCTGCA | |
| | | | | GCGACGCGTTATCCACTACT | |
| | | | | ACAACTGTTGAACAACTATC | |
| | | | | AAACACCCCAACGACCAATC | |
| HPV51 | SD1-SA4 | 199 | 200 | GTTTCTATGCACAATATACA | 1600 |
| | i = 4 | | | GGTAGTGTGTGTGTATTGTA | |
| | | | | AAAAGGAATTATGTAGAGCA | |
| | | | | GGATGCAAACCTAATGTATT | |
| | | | | TACATACAAGGGTAACAGTA | |
| | | | | TTAAAGTTTTTAAATACATT | |
| | | | | TCCATTTGATAACA | |
| HPV51 | SD1-SA6 | 201 | 202 | GTTTCTATGCACAATATACA | 1601 |
| | i = 6 | | | GGTAGTGTGTGTGTATTGTA | |
| | | | | AAAAGGAATTATGTAGAGCA | |
| | | | | GTACCTGCAGCGACGCGTTA | |
| | | | | TCCACTACTACAACTGTTGA | |
| | | | | ACAACTATCAAACACCCCAA | |
| | | | | CGACCAATC | |
| HPV51 | SD1-SA1 | 203 | 204 | GTTTCTATGCACAATATACA | 1602 |
| | i = 7 | | | GGTAGTGTGTGTGTATTGTA | |
| | | | | AAAAGGAATTATCiTAGAGC | |
| | | | | AGGTGTCATAGATGTCAAAG | |
| | | | | ACCACTTGGGCCTGAAGAAA | |
| | | | | AGCAAAAATTGGTGGACGAA | |
| | | | | AAA | |
| HPV51 | SD1-SA3 | 205 | 206 | GTTTCTATGCACAATATACA | 1603 |
| | i = 9 | | | GGTAGTGTGTGTGTATTGTA | |
| | | | | AAAAGGAATTATGTAGAGCA | |
| | | | | GGTGTTCAAGTGTAGTACAA | |
| | | | | CTGGCAGTGGAAAGCAGTGG | |
| | | | | AGACACC | |
| HPV51 | SD5-SA9 | 207 | 208 | GTGCAACTCAGACTGCGTTT | 1604 |
| | i = 11 | | | ATAGTGCATTTAAAAGATGG | |
| | | | | CATTGTGGCGCACTAATGAC | |
| | | | | AGCAAGGTGTATTTGCCAC | |
| HPV51 | SD2-SA4 | 209 | 210 | GCCCGTGTTGTGCGAACAAC | 1605 |
| | i = 15 | | | TAGCAACGGCGATGGACTGT | |
| | | | | GAAGGATGCAAACCTAATGT | |
| | | | | ATTTACATACAAGGGTAACA | |
| | | | | GTATTAAAGTTTTTAAATAC | |
| | | | | ATTTCCATTTCiATAACA | |
| HPV51 | SD2-SA6 | 211 | 212 | GCCCGTGTTGTGCGAACAAC | 1606 |
| | i = 17 | | | TAGCAACGGCGATGGACTGT | |
| | | | | GAAGTACCTGCAGCGACGCG | |
| | | | | TTATCCACTACTACAACTGT | |
| | | | | TGAACAACTATCAAACACCC | |
| | | | | CAACGACCAATC | |
| HPV51 | SD2-SA9 | 213 | 214 | GCCCGTGTTGTGCGAACAAC | 1607 |
| | i = 18 | | | TAGCAACGGCGATGGACTGT | |
| | | | | GAAGATGGCATTGTGGCGCA | |
| | | | | CTAATGACAGCAAGGTGTAT | |
| | | | | TTGCCAC | |
| HPV52 | SD3-SA4 | 215 | 216 | CGGCTATGGCAATAGTGAAG | 1608 |
| | i = 1 | | | TGGAAGCGCAGCAGATGGCA | |
| | | | | GACCAGATCCTAGGTGGCCA | |
| | | | | TATTTACATAGTA | |
| HPV52 | SD3-SA5 | 217 | 218 | CGGCTATGGCAATAGTGAAG | 1609 |
| | i = 2 | | | TGGAAGCGCAGCAGATGGCA | |
| | | | | GACCAGGACGTGGTGCAAAT | |
| | | | | TAGATTTAATACAGGAAGAG | |
| | | | | GACAA | |
| HPV52 | SD3-SA6 | 219 | 220 | CGGCTATGGCAATAGTGAAG | 1610 |
| | i = 3 | | | TGGAAGCGCAGCAGATGGCA | |
| | | | | GACCAGTAACGAAGTATCCA | |
| | | | | CTACTGAAACTGCTGTCCAC | |
| | | | | CTATGCACCG | |
| HPV52 | SD1-SA4 | 221 | 222 | GCAGTGTGTGCAGTGCAAAA | 1611 |
| | i = 4 | | | AAGAGCTACAACGAAGAGAG | |
| | | | | ATCCTAGGTGGCCATATTTA | |
| | | | | CATAGTA | |
| HPV52 | SD1-SA5 | 223 | 224 | GCAGTGTGTGCAGTGCAAAA | 1612 |
| | i = 5 | | | AAGAGCTACAACGAAGAGAG | |
| | | | | GACGTGGTGCAAATTAGATT | |
| | | | | TAATACAGGAAGAGGACAA | |
| HPV52 | SD1-SA6 | 225 | 226 | GCAGTGTGTGCAGTGCAAAA | 1613 |
| | i = 6 | | | AAGAGCTACAACGAAGAGAG | |
| | | | | TAACGAAGTATCCACTACTG | |
| | | | | AAACTGCTGTCCACCTATGC | |
| | | | | ACCG | |
| HPV52 | SD1-SA1 | 227 | 228 | GCAGTGTGTGCAGTGCAAAA | 1614 |
| | i = 7 | | | AAGAGCTACAACGAAGAGAG | |
| | | | | ATGTATAATTTGTCAAACGC | |
| | | | | CATTATGTCCTGAAGAAAA | |
| HPV52 | SD1-SA2 | 229 | 230 | GCACiTGTGTGCAGTGCAAA | 1615 |
| | i = 8 | | | AAAGAGCTACAACGAAGAGA | |
| | | | | GACCCCGACCTGTGACCCAA | |
| | | | | GTGTAACGTCATGCGTGGAG | |
| | | | | ACAAAGCAACTATAAAAGAT | |
| | | | | TATA | |
| HPV52 | SD1-SA3 | 231 | 232 | GCAGTGTGTGCAGTGCAAAA | 1616 |
| | i = 9 | | | AAGAGCTACAACGAAGAGAG | |
| | | | | TTGTGATAGCACACTACGGC | |
| | | | | TATGCATTCATAGCACTGCG | |
| | | | | ACGG | |
| HPV52 | SD5-SA9 | 233 | 234 | ACAAAGGACGGGTTGCACAT | 1617 |
| | i = 11 | | | ACAACTTGTACTGCACCTAT | |
| | | | | AATACACCTAAAAGATGTCC | |
| | | | | GTGTGGCGGCCTAGTGAGGC | |
| | | | | CACTGTGTACCTGCCTCC | |
| HPV52 | SD5-SA10 | 235 | 236 | ACAAAGGACGGGTTGCACAT | 1618 |
| | i = 12 | | | ACAACTTGTACTGCACCTAT | |
| | | | | AATACACCTAAAAGTAGTGG | |
| | | | | TAATGGTAAAAAAGTTTTAG | |
| | | | | TTCCCAAGG | |
| HPV52 | SD2-SA4 | 237 | 238 | GTGTGCCCCGGCTGTGCACG | 1619 |
| | i = 15 | | | GCTATAAACAACCCTGCAAT | |
| | | | | GGAGGACCCTGAAGATCCTA | |
| | | | | GGTGGCCATATTTACATAGT | |
| | | | | A | |
| HPV52 | SD2-SA5 | 239 | 240 | GTGTGCCCCGGCTGTGCACG | 1620 |
| | i = 16 | | | GCTATAAACAACCCTGCAAT | |
| | | | | GGAGGACCCTGAAGGACGTG | |
| | | | | GTGCAAATTAGATTTAATAC | |
| | | | | AGGAAGAGGACAA | |
| HPV52 | SD2-SA6 | 241 | 242 | GTGTGCCCCGGCTGTGCACG | 1621 |
| | i = 17 | | | GCTATAAACAACCCTGCAAT | |
| | | | | GGAGGACCCTGAAGTAACGA | |
| | | | | AGTATCCACTACTGAAACTG | |
| | | | | CTGTCCACCTATGCACCG | |
| HPV52 | SD2-SA9 | 243 | 244 | GTGTGCCCCGGCTGTGCACG | 1622 |
| | i = 18 | | | GCTATAAACAACCCTGCAAT | |
| | | | | GGAGGACCCTCiAAGATGTC | |
| | | | | CGTGTGGCGGCCTAGTGAGG | |
| | | | | CCACTGTGTACCTGCCTCC | |
| HPV52 | SD2-SA10 | 245 | 246 | GTGTGCCCCGGCTGTGCACG | 1623 |
| | i = 19 | | | GCTATAAACAACCCTGCAAT | |
| | | | | GGAGGACCCTGAAGTAGTGG | |
| | | | | TAATGGTAAAAAAGTTTTAG | |
| | | | | TTCCCAAGG | |
| HPV56 | SD3-SA4 | 247 | 248 | CATTGGAAACTCTGGAAACA | 1624 |
| | i = 1 | | | CCAGAACAGATGCTAAATTA | |
| | | | | CGATATTT | |
| HPV56 | SD3-SA5 | 249 | 250 | CATTGGAAACTCTGGAAACA | 1625 |
| | i = 2 | | | CCAGAACAGGACGTGGTCCA | |
| | | | | GATTAAAT | |
| HPV56 | SD3-SA6 | 251 | 252 | CATTGGAAACTCTGGAAACA | 1626 |
| | i = 3 | | | CCAGAACAGTACCTGTAGAT | |
| | | | | ACAACGTATCCCCTGTTGAA | |
| | | | | ACTGTTAACGAATACAACAC | |
| | | | | CCAC | |
| HPV56 | SD1-SA4 | 253 | 254 | ATACCTTTAATTGATCTTAG | 1627 |
| | i = 4 | | | ATTATCATGTGTATATTGCA | |
| | | | | AAAAAGAACTAACACGTGCT | |
| | | | | GAGATGCTAAATTACGATAT | |
| | | | | TT | |
| HPV56 | SD1-SA5 | 255 | 256 | ATACCTTTAATTGATCTTAG | 1628 |
| | i = 5 | | | ATTATCATGTGTATATTGCA | |
| | | | | AAAAAGAACTAACACGTGCT | |
| | | | | GAGGACGTGGTCCAGATTAA | |
| | | | | AT | |
| HPV56 | SD1-SA6 | 257 | 258 | ATACCTTTAATTGATCTTAG | 1629 |
| | i = 6 | | | ATTATCATGTGTATATTGCA | |
| | | | | AAAAAGAACTAACACGTGCT | |
| | | | | GAGTACCTGTAGATACAACG | |
| | | | | TATCCCCTGTTGAAACTGTT | |
| | | | | AACGAATACAACACCCAC | |
| HPV56 | SD1-SA1 | 259 | 260 | ATACCTTTAATTGATCTTAG | 1630 |
| | i = 7 | | | ATTATCATGTGTATATTGCA | |
| | | | | AAAAAGAACTAACACGTGCT | |
| | | | | GAGGTGCTACAGATGTCAAA | |
| | | | | GTCCG | |
| HPV56 | SD1-SA2 | 261 | 262 | ATACCTTTAATTGATCTTAG | 1631 |
| | i = 8 | | | ATTATCATGTGTATATTGCA | |
| | | | | AAAAAGAACTAACACGTGCT | |
| | | | | GAGACAAACATCTAGAGAAC | |
| | | | | CTAGAGAATCTACAGTATAA | |
| | | | | TCATGCATGGTAAAG | |
| HPV56 | SD1-SA3 | 263 | 264 | ATACCTTTAATTGATCTTAG | 1632 |
| | i = 9 | | | ATTATCATGTGTATATTGCA | |
| | | | | AAAAAGAACTAACACGTGCT | |
| | | | | GAGTGTAAGTTTGTGGTGCA | |
| | | | | GTTGGACATTCAGAGTACCA | |
| | | | | AA | |
| HPV56 | SD5-SA9 | 265 | 266 | ACTACGCCTGTAGTACATTT | 1633 |
| | i = 11 | | | AAAAGATGGCGACGTGGCGG | |
| | | | | CCTAGTGAAAATAAGGTGTA | |
| | | | | TCTACC | |
| HPV56 | SD5-SA10 | 267 | 268 | ACTACGCCTGTAGTACATTT | 1634 |
| | i = 12 | | | AAAAGGACAATACCAAAACA | |
| | | | | AACATTCCCAAAGTTAGTGC | |
| | | | | ATA | |
| HPV56 | SD2-SA4 | 269 | 270 | GCGCATCAAGTAACTAACTG | 1635 |
| | i = 15 | | | CAATGGCGTCACCTGAAGAT | |
| | | | | GCTAAATTACGATATTT | |
| HPV56 | SD2-SA5 | 271 | 272 | GCGCATCAAGTAACTAACTG | 1636 |
| | i = 16 | | | CAATGGCGTCACCTGAAGGA | |
| | | | | CGTGGTCCAGATTAAAT | |
| HPV56 | SD2-SA6 | 273 | 274 | GCGCATCAAGTAACTAACTG | 1637 |
| | i = 17 | | | CAATGGCGTCACCTGAAGTA | |
| | | | | CCTGTAGATACAACGTATCC | |
| | | | | CCTGTTGAAACTGTTAACGA | |
| | | | | ATACAACACCCAC | |
| HPV56 | SD2-SA9 | 275 | 276 | GCGCATCAAGTAACTAACTG | 1638 |
| | i = 18 | | | CAATGGCGTCACCTGAAGAT | |
| | | | | GGCGACGTGGCGGCCTAGTG | |
| | | | | AAAATAAGGTGTATCTACC | |
| HPV56 | SD2-SA10 | 277 | 278 | GCGCATCAAGTAACTAACTG | 1639 |
| | i = 19 | | | CAATGGCGTCACCTGAAGGA | |
| | | | | CAATACCAAAACAAACATTC | |
| | | | | CCAAAGTTAGTGCATA | |
| HPV58 | SD3-SA4 | 279 | 280 | ATGGCAATACTGAAGTGGAA | 1640 |
| | i = 1 | | | ACTGAGCAGATGGCACACCA | |
| | | | | GATTCACGATGGCCATATTT | |
| | | | | GCACAGTAGACTAACAGTAT | |
| | | | | T | |
| HPV58 | SD3-SA5 | 281 | 282 | ATGGCAATACTGAAGTGGAA | 1641 |
| | i = 2 | | | ACTGAGCAGATGGCACACCA | |
| | | | | GGACGTGGTGCAAATTAGGC | |
| | | | | TTAATAGAGGAAGAG | |
| HPV58 | SD3-SA6 | 283 | 284 | ATGGCAATACTGAAGTGGAA | 1642 |
| | i = 3 | | | ACTGAGCAGATGGCACACCA | |
| | | | | GTGATCAAATATCCACTACT | |
| | | | | GAAACTGCTGACCCAAAGAC | |
| | | | | CACCGAGGCC | |
| HPV58 | SD1-SA4 | 285 | 286 | GTGCATGAAATCGAATTGAA | 1643 |
| | i = 4 | | | ATGCGTTGAATGCAAAAAGA | |
| | | | | CTTTGCAGCGATCTGAGATT | |
| | | | | CACGATGGCCATATTTGCAC | |
| | | | | AGTAGACTAACAGTATT | |
| HPV58 | SD1-SA5 | 287 | 288 | GTGCATGAAATCGAATTGAA | 1644 |
| | i = 5 | | | ATGCGTTGAATGCAAAAAGA | |
| | | | | CTTTGCAGCGATCTGAGGAC | |
| | | | | GTGGTGCAAATTAGGCTTAA | |
| | | | | TAGAGGAAGAG | |
| HPV58 | SD1-SA6 | 289 | 290 | GTGCATGAAATCGAATTGAA | 1645 |
| | i = 6 | | | ATGCGTTGAATGCAAAAAGA | |
| | | | | CTTTGCAGCGATCTGAGTGA | |
| | | | | TCAAATATCCACTACTGAAA | |
| | | | | CTGCTGACCCAAAGACCACC | |
| | | | | GAGGCC | |
| HPV58 | SD1-SA1 | 291 | 292 | GTGCATGAAATCGAATTGAA | 1646 |
| | i = 7 | | | ATGCGTTGAATGCAAAAAGA | |
| | | | | CTTTGCAGCGATCTGAGATG | |
| | | | | TATTATTTGTCAAAGACCAT | |
| | | | | TGTGTCCACAAGAAAAAAAA | |
| | | | | AGGCATGTGGATTTAA | |
| HPV58 | SD1-SA2 | 293 | 294 | GTGCATGAAATCGAATTGAA | 1647 |
| | i = 8 | | | ATGCGTTGAATGCAAAAAGA | |
| | | | | CTTTGCAGCGATCTGAGACC | |
| | | | | CCGACGTAGACAAACACAAG | |
| | | | | TGTAACCTGTAACAACGCCA | |
| HPV58 | SD5-SA9 | 295 | 296 | GTACATACAAAGGGCGGAAC | 1648 |
| | i = 11 | | | GTGTGTAGTTCTAAAGTTTC | |
| | | | | ACCTATCGTGCATTTAAAAG | |
| | | | | ATGTCCGTGTGGCGGCCTAG | |
| | | | | TGAGGCCACTGTGTACCTGC | |
| | | | | CTCCTG | |
| HPV58 | SD2-SA4 | 297 | 298 | GTGTGCCCTAGCTGTGCACA | 1649 |
| | i = 15 | | | GCAATAAACACCATCTGCAA | |
| | | | | TGGATGACCCTGAAGATTCA | |
| | | | | CGATGGCCATATTTGCACAG | |
| | | | | TAGACTAACAGTATT | |
| HPV58 | SD2-SA5 | 299 | 300 | GTGTGCCCTAGCTGTGCACA | 1650 |
| | i = 16 | | | GCAATAAACACCATCTGCAA | |
| | | | | TGGATGACCCTGAAGGACGT | |
| | | | | GGTGCAAATTAGGCTTAATA | |
| | | | | GAGGAAGAG | |
| HPV58 | SD2-SA6 | 301 | 302 | GTGTGCCCTAGCTGTGCACA | 1651 |
| | i = 17 | | | GCAATAAACACCATCTGCAA | |
| | | | | TGGATGACCCTGAAGTGATC | |
| | | | | AAATATCCACTACTGAAACT | |
| | | | | GCTGACCCAAAGACCACCGA | |
| | | | | GGCC | |
| HPV58 | SD2-SA9 | 303 | 304 | GTGTGCCCTAGCTGTGCACA | 1652 |
| | i = 18 | | | GCAATAAACACCATCTGCAA | |
| | | | | TGGATGACCCTGAAGATGTC | |
| | | | | CGTGTGGCGGCCTAGTGAGG | |
| | | | | CCACTGTGTACCTGCCTCCT | |
| | | | | G | |
| HPV59 | SD3-SA4 | 305 | 306 | GCGGCTATGGCTATTCTGAA | 1653 |
| | i = 1 | | | GTGGAAATGCTCGAGACTCA | |
| | | | | GATAACAGGTGGCCATATTT | |
| | | | | AAATAGCAGATTAATGGTAT | |
| | | | | TTAAATT | |
| HPV59 | SD3-SA5 | 307 | 308 | GCGGCTATGGCTATTCTGAA | 1654 |
| | i = 2 | | | GTGGAAATGCTCGAGACTCA | |
| | | | | GGACGTGGTGCAGATTAGAT | |
| | | | | TTGAACGAGGAAGAGGAAG | |
| HPV59 | SD3-SA7 | 309 | 310 | GCGGCTATGGCTATTCTGAA | 1655 |
| | i = 22 | | | GTGGAAATGCTCGAGACTCA | |
| | | | | GTGACCiAGCAAGTATCCAC | |
| | | | | TGCTGGATCTTCTGAGCAAC | |
| | | | | TATCATACCCCTCCGCAACG | |
| | | | | CCCC | |
| HPV59 | SD1-SA4 | 311 | 312 | GGAACTGCAAGAAAGAGAGA | 1656 |
| | i = 4 | | | TAACAGGTGGCCATATTTAA | |
| | | | | ATAGCAGATTAATGGTATTT | |
| | | | | AAATT | |
| HPV59 | SD1-SA5 | 313 | 314 | GGAACTGCAAGAAAGAGAGG | 1657 |
| | i = 5 | | | ACGTGGTGCAGATTAGATTT | |
| | | | | GAACGAGGAAGAGGAAG | |
| HPV59 | SD1-SA7 | 315 | 316 | GGAACTGCAAGAAAGAGAGT | 1658 |
| | i = 20 | | | GACGAGCAAGTATCCACTGC | |
| | | | | TGGATCTTCTGAGCAACTAT | |
| | | | | CATACCCCTCCGCAACGCCC | |
| | | | | C | |
| HPV59 | SD1-SA3 | 317 | 318 | GGAACTGCAAGAAAGAGAGT | 1659 |
| | i = 9 | | | GTAATAATCAACTTCAGCTA | |
| | | | | GTAGTAGAAACCTCGCAAG | |
| HPV59 | SD5-SA9 | 319 | 320 | CAACCCGCGACGGCACATCC | 1660 |
| | i = 11 | | | CTTGCAGTAACACTACGCCT | |
| | | | | ATAATACACTTAAAAGATGG | |
| | | | | CTCTATGGCGTTCTAGTGAC | |
| | | | | AACAAGGTGTATCTACCTCC | |
| | | | | ACCTTCGGTAGCTAAG | |
| HPV59 | SD5-SA10 | 321 | 322 | CAACCCGCGACGGCACATCC | 1661 |
| | i = 12 | | | CTTGCAGTAACACTACGCCT | |
| | | | | ATAATACACTTAAAAGGTGG | |
| | | | | TAATGGTAGACAGGATGTTC | |
| | | | | CTAAGGTGTCTGCATATCAA | |
| | | | | TACAGAGTATTTAGGGTT | |
| HPV59 | SD2-SA4 | 323 | 324 | GCAGCAAACCAGTAACCTGC | 1662 |
| | i = 15 | | | AATGGCCGATTCGGAAGATA | |
| | | | | ACAGGTGGCCATATTTAAAT | |
| | | | | AGCAGATTAATGGTATTTAA | |
| | | | | ATT | |
| HPV59 | SD2-SA5 | 325 | 326 | GCAGCAAACCAGTAACCTGC | 1663 |
| | i = 16 | | | AATGGCCGATTCGGAAGGAC | |
| | | | | GTGGTGCAGATTAGATTTGA | |
| | | | | ACGAGGAAGAGGAAG | |
| HPV59 | SD2-SA7 | 327 | 328 | GCAGCAAACCAGTAACCTGC | 1664 |
| | i = 21 | | | AATGGCCGATTCGGAAGTGA | |
| | | | | CGAGCAAGTATCCACTGCTG | |
| | | | | GATCTTCTGAGCAACTATCA | |
| | | | | TACCCCTCCGCAACGCCCC | |
| HPV59 | SD2-SA9 | 329 | 330 | GCAGCAAACCAGTAACCTGC | 1665 |
| | i = 18 | | | AATGGCCGATTCGGAAGATG | |
| | | | | GCTCTATGGCGTTCTAGTGA | |
| | | | | CAACAAGGTGTATCTACCTC | |
| | | | | CACCTTCGGTAGCTAAG | |
| HPV59 | SD2-SA10 | 331 | 332 | GCAGCAAACCAGTAACCTGC | 1666 |
| | i = 19 | | | AATGGCCGATTCGGAAGGTG | |
| | | | | GTAATGGTAGACAGGATGTT | |
| | | | | CCTAAGGTGTCTGCATATCA | |
| | | | | ATACAGAGTATTTAGGGTT | |
| HPV66 | SD3-SA4 | 333 | 334 | CATTGGAAACATTGGAAACA | 1667 |
| | i = 1 | | | TCACAACAGATGCAAAATTA | |
| | | | | AGATATTTACACAGTAGAAT | |
| | | | | TTCAGTGTTTAAGTTTGAAA | |
| | | | | ATCCATTTCCATTAGATAAC | |
| HPV66 | SD3-SA5 | 335 | 336 | CATTGGAAACATTGGAAACA | 1668 |
| | i = 2 | | | TCACAACAGGACATGGTCCA | |
| | | | | GATTAAAT | |
| HPV66 | SD3-SA6 | 337 | 338 | GCAAGTACAAACAGCACATG | 1669 |
| | i = 3 | | | CAGATGCACAGACGTTGCAA | |
| | | | | AAACTAAAACGAAAGTATAT | |
| | | | | AGGTAGTCCCTTAAGTGATA | |
| | | | | TTAG | |
| HPV66 | SD3-SA8 | 339 | 340 | CATTGGAAACATTGGAAACA | 1670 |
| | i = 23 | | | TCACAACAGACTGTTAACGA | |
| | | | | ATACAACAAC | |
| HPV66 | SD1-SA4 | 341 | 342 | AATACCTTTACTTGATCTTA | 1671 |
| | i = 4 | | | GATTATCATGTGTATACTGC | |
| | | | | AAAAAGGAACTTACAAGTTT | |
| | | | | AGAGATGCAAAATTAAGATA | |
| | | | | TTTACACAGTAGAATTTCAG | |
| | | | | TGTTTAAGTTTGAAAATCCA | |
| | | | | TTTCCATTAGATAAC | |
| HPV66 | SD1-SA5 | 343 | 344 | AATACCTTTACTTGATCTTA | 1672 |
| | i = 5 | | | GATTATCATGTGTATACTGC | |
| | | | | AAAAAGGAACTTACAAGTTT | |
| | | | | AGAGGACATGGTCCAGATTA | |
| | | | | AAT | |
| HPV66 | SD1-SA8 | 345 | 346 | AATACCTTTACTTGATCTTA | 1673 |
| | i = 24 | | | GATTATCATGTGTATACTGC | |
| | | | | AAAAAGGAACTTACAAGTTT | |
| | | | | AGAGACTGTTAACGAATACA | |
| | | | | ACAAC | |
| HPV66 | SD1-SA1 | 347 | 348 | AATACCTTTACTTGATCTTA | 1674 |
| | i = 7 | | | GATTATCATGTGTATACTGC | |
| | | | | AAAAAGGAACTTACAAGTTT | |
| | | | | AGAGGTGCTACCGATGTCAA | |
| | | | | TGTCCGTTAACACCGGAGGA | |
| | | | | AAAACAA | |
| HPV66 | SD1-SA2 | 349 | 350 | AATACCTTTACTTGATCTTA | 1675 |
| | i = 8 | | | GATTATCATGTGTATACTGC | |
| | | | | AAAAAGGAACTTACAAGTTT | |
| | | | | AGAGACATACGAGTAGACAA | |
| | | | | GCTACAGAATCTACAGTATA | |
| | | | | ACCATGCATGGTAA | |
| HPV66 | SD1-SA3 | 351 | 352 | AATACCTTTACTTGATCTTA | 1676 |
| | i = 9 | | | GATTATCATGTGTATACTGC | |
| | | | | AAAAAGGAACTTACAAGTTT | |
| | | | | AGAGTGTGAGTTGGTGGTGC | |
| | | | | AGTTGGACATTCAGAGTACC | |
| | | | | AA | |
| HPV66 | SD5-SA9 | 353 | 354 | GGTGATAAAACTACGCCTGT | 1677 |
| | i = 11 | | | AATCCATTTAAAAGATGGCG | |
| | | | | ATGTGGCGGCCTAGTGACAA | |
| | | | | TAAGGTGTACCTACC | |
| HPV66 | SD2-SA4 | 355 | 356 | GCGCATCATCTAAATAACTG | 1678 |
| | i = 15 | | | CAATGGCATCACCTGAAGAT | |
| | | | | GCAAAATTAAGATATTTACA | |
| | | | | CAGTAGAATTTCAGTGTTTA | |
| | | | | AGTTTGAAAATCCATTTCCA | |
| | | | | TTAGATAAC | |
| HPV66 | SD2-SA5 | 357 | 358 | GCGCATCATCTAAATAACTG | 1679 |
| | i = 16 | | | CAATGGCATCACCTGAAGGA | |
| | | | | CATGGTCCAGATTAAAT | |
| HPV66 | SD2-SA8 | 359 | 360 | GCGCATCATCTAAATAACTG | 1680 |
| | i = 25 | | | CAATGGCATCACCTGAAGAC | |
| | | | | TGTTAACGAATACAACAAC | |
| HPV66 | SD2-SA9 | 361 | 362 | GCGCATCATCTAAATAACTG | 1681 |
| | i = 18 | | | CAATGGCATCACCTGAAGAT | |
| | | | | GGCGATGTGGCGGCCTAGTG | |
| | | | | ACAATAAGGTGTACCTACC | |
| HPV68 | SD3-SA4 | 363 | 364 | CCGGACAGCGGCTATGGCAA | 1682 |
| | i = 1 | | | TATGGAAGTGGAAACTAACT | |
| | | | | CGGAGACAATACiGTGGCCG | |
| | | | | TATTTACATAGTAGACTAAC | |
| | | | | CGTGTTTAAATTTC | |
| HPV68 | SD3-SA6 | 365 | 366 | CCGGACAGCGGCTATGGCAA | 1683 |
| | i = 3 | | | TATGGAAGTGGAAACTAACT | |
| | | | | CGGAGTACCACTGACGGAAA | |
| | | | | AGTATCCACTACTGAATCTG | |
| | | | | TTGCCGAC | |
| HPV68 | SD1-SA4 | 367 | 368 | GTTACAATAGACTGTGTCTA | 1684 |
| | i = 4 | | | TTGCAGAAGGCAACTACAAC | |
| | | | | GGACAGAGACAATAGGTGGC | |
| | | | | CGTATTTACATAGTAGACTA | |
| | | | | ACCGTGTTTAAATTTC | |
| HPV68 | SD1-SA1 | 369 | 370 | GTTACAATAGACTGTGTCTA | 1685 |
| | i = 7 | | | TTGCAGAAGGCAACTACAAC | |
| | | | | GGACAGAGGTGCATGAGTTG | |
| | | | | CCTGAAACCATTGTGTCCAG | |
| | | | | CAGAAAAA | |
| HPV68 | SD1-SA6 | 371 | 372 | GTTACAATAGACTGTGTCTA | 1686 |
| | i = 6 | | | TTGCAGAAGGCAACTACAAC | |
| | | | | GGACAGAGTACCACTGACCi | |
| | | | | GAAAAGTATCCACTACTGAA | |
| | | | | TCTGTTGCCGAC | |
| HPV68 | SD1-SA3 | 373 | 374 | GTTACAATAGACTGTGTCTA | 1687 |
| | i = 9 | | | TTGCAGAAGGCAACTACAAC | |
| | | | | GGACAGAGTGTAACAAGGCA | |
| | | | | CTGCAACTAGTAGTAGAAGC | |
| | | | | GTCGCGGGAC | |
| HPV68 | SD5-SA9 | 375 | 376 | AAGACGGAGCCTTTGTTGTG | 1688 |
| | i = 11 | | | GTGACACTACACCTATAGTG | |
| | | | | CATTTAAAAGATGGCATTGT | |
| | | | | GGCGAGCTAGCGACAACATG | |
| | | | | GTGTATTTGCCTCCCCCC | |
| HPV68 | SD2-SA4 | 377 | 378 | ACCCAGTAATCTGCAATGGC | 1689 |
| | i = 15 | | | CAATTGTGAAGACAATAGGT | |
| | | | | GGCCGTATTTACATAGTAGA | |
| | | | | CTAACCGTGTTTAAATTTC | |
| HPV68 | SD2-SA6 | 379 | 380 | ACCCAGTAATCTGCAATGGC | 1690 |
| | i = 17 | | | CAATTGTGAAGTACCACTGA | |
| | | | | CGGAAAAGTATCCACTACTG | |
| | | | | AATCTGTTGCCGAC | |
| HPV68 | SD2-SA9 | 381 | 382 | ACCCAGTAATCTGCAATGGC | 1691 |
| | i = 18 | | | CAATTGTGAAGATGGCATTG | |
| | | | | TGGCGAGCTAGCGACAACAT | |
| | | | | GGTGTATTTGCCTCCCCCC | |
| HPV73 | SD3-SA4 | 383 | 384 | GGACAGTGGATATGGCAATA | 1692 |
| | i = 1 | | | CTGAAGTGGAAACTTACGAG | |
| | | | | ACAGAGATGATACTTGGAAA | |
| | | | | TATTTACATAGTAGAATTAA | |
| | | | | GGTGTTTACTTTTTTAAATC | |
| | | | | CATT | |
| HPV73 | SD3-SA6 | 385 | 386 | GGACAGTGGATATGGCAATA | 1693 |
| | i = 3 | | | CTGAAGTGGAAACTTACGAG | |
| | | | | ACAGAGCGCCTGTGAAGTAT | |
| | | | | CCATTCCTGAAATTGTTAAC | |
| | | | | CCACTGCAC | |
| HPV73 | SD1-SA4 | 387 | 388 | ATACATGATATAAACCTGGA | 1694 |
| | i = 4 | | | CTGTGTGTTTTGCCAACGTG | |
| | | | | GACTGTACAGATCTGAGATG | |
| | | | | ATACTTGGAAATATTTACAT | |
| | | | | AGTAGAATTAAG | |
| HPV73 | SD1-SA6 | 389 | 390 | ATACATGATATAAACCTGGA | 1695 |
| | i = 6 | | | CTGTGTGTTTTGCCAACGTG | |
| | | | | GACTGTACAGATCTGAGCGC | |
| | | | | CTGTGAAGTATCCATTCCTG | |
| | | | | AAATTGTTAACCCACTGCAC | |
| HPV73 | SD1-SA1 | 391 | 392 | ATACATGATATAAACCTGGA | 1696 |
| | i = 7 | | | CTGTGTGTTTTGCCAACGTG | |
| | | | | GACTGTACAGATCTGAGGTG | |
| | | | | CGGAAAATGCCAAAAACCAT | |
| | | | | TATGTCCACTGGAAAAGCAA | |
| | | | | AAGCATGTAGATGAAAA | |
| HPV73 | SD1-SA2 | 393 | 394 | ATACATGATATAAACCTGGA | 1697 |
| | i = 8 | | | CTGTGTGTTTTGCCAACGTG | |
| | | | | GACTGTACAGATCTGAGACC | |
| | | | | ATCTGCAACTGTGGTGTAAG | |
| | | | | ATGCATGGAAAAAAAACAAC | |
| | | | | CTTGCAGGACATTACT | |
| HPV73 | SD1-SA3 | 395 | 396 | ATACATGATATAAACCTGGA | 1698 |
| | i = 9 | | | CTGTGTGTTTTGCCAACGTG | |
| | | | | GACTGTACAGATCTGAGTGT | |
| | | | | CAGTGCACAGTATGCCTTGC | |
| | | | | CATTGAAAGCAACAAAGCTG | |
| | | | | ATTTAAGAGTGA | |
| HPV73 | SD5-SA9 | 397 | 398 | CCTGTACCCAGTGTACTACA | 1699 |
| | i = 11 | | | CATAATGTTGCGCCAATAGT | |
| | | | | GCATTTAAAAGATGTGGCGA | |
| | | | | CCTACTGATGCAAAGGTATA | |
| | | | | CCTGCCCCC | |
| HPV73 | SD5-SA10 | 399 | 400 | CCTGTACCCAGTGTACTACA | 1700 |
| | i = 12 | | | CATAATGTTGCGCCAATAGT | |
| | | | | GCATTTAAAAGGATTCTCAA | |
| | | | | AAACGTAAAACCATAGTTCC | |
| | | | | TAAAGTTTCAGGTTTG | |
| HPV73 | SD2-SA4 | 401 | 402 | GCCCCAACTGTTCCAGAAAC | 1701 |
| | i = 15 | | | CTATAAAAGAAGATGGCTGA | |
| | | | | TTCAGATGATACTTGGAAAT | |
| | | | | ATTTACATAGTAGAATTAAG | |
| | | | | GTGTTTACTTTTTTAAATCC | |
| | | | | ATT | |
| HPV73 | SD2-SA6 | 403 | 404 | GCCCCAACTGTTCCAGAAAC | 1702 |
| | i = 17 | | | CTATAAAAGAAGATGGCTGA | |
| | | | | TTCAGCGCCTGTGAAGTATC | |
| | | | | CATTCCTGAAATTGTTAACC | |
| | | | | CACTGCAC | |
| HPV73 | SD2-SA9 | 405 | 406 | GCCCCAACTGTTCCAGAAAC | 1703 |
| | i = 18 | | | CTATAAAAGAAGATGGCTGA | |
| | | | | TTCAGATGTGGCGACCTACT | |
| | | | | GATGCAAAGGTATACCTGCC | |
| | | | | CCC | |
| HPV73 | SD2-SA10 | 407 | 408 | GCCCCAACTGTTCCAGAAAC | 1704 |
| | i = 19 | | | CTATAAAAGAAGATGGCTGA | |
| | | | | TTCAGGATTCTCAAAAACGT | |
| | | | | AAAACCATAGTTCCTAAAGT | |
| | | | | TTCAGGTTTG | |
| HPV82 | SD3-SA4 | 409 | 410 | CACAAGTGGAGACTGTGGAA | 1705 |
| | i = l | | | GGACCCTTACAGATCCAAAT | |
| | | | | TTAATGTATTTACATAGTAG | |
| | | | | AGTGACAGTATTTCAATTTT | |
| | | | | TAAATGCATTTCCATTTGAC | |
| | | | | CCCCAT | |
| HPV82 | SD3-SA6 | 411 | 412 | CACAAGTGGAGACTGTGGAA | 1706 |
| | i = 3 | | | GGACCCTTACAGTACCTACA | |
| | | | | GCACCCCGTCACCCTCTACT | |
| | | | | ACAACTG | |
| HPV82 | SD1-SA4 | 413 | 414 | ATTCAGGTATTGTGTGTATA | 1707 |
| | i = 4 | | | TTGTAAAAAGGAGTTGTGTA | |
| | | | | GAGCAGATCCAAATTTAATG | |
| | | | | TATTTACATAGTAGAGTGAC | |
| | | | | AGTATTTCAATTTTTAAATG | |
| | | | | CATTTCCATTTGACCCCCAT | |
| HPV82 | SD1-SA6 | 415 | 416 | ATTCAGGTATTGTGTGTATA | 1708 |
| | i = 6 | | | TTGTAAAAAGGAGTTGTGTA | |
| | | | | GAGCAGTACCTACAGCACCC | |
| | | | | CGTCACCCTCTACTACAACT | |
| | | | | G | |
| HPV82 | SD1-SA1 | 417 | 418 | ATTCAGGTATTGTGTGTATA | 1709 |
| | i = 7 | | | TTGTAAAAAGGAGTTGTGTA | |
| | | | | GAGCAGGTGTCATAGATGTC | |
| | | | | AGAGACCACTTGGGCCTGAA | |
| | | | | GAAAAG | |
| HPV82 | SD1-SA2 | 419 | 420 | ATTCAGGTATTGTGTGTATA | 1710 |
| | i = 8 | | | TTGTAAAAAGGAGTTGTGTA | |
| | | | | GAGCAGAAAACCACCAAGAC | |
| | | | | AACGTAGTGAAACCCAGGTG | |
| | | | | TAATAACGCCATGCGTGGTA | |
| | | | | ATGT | |
| HPV82 | SD1-SA3 | 421 | 422 | ATTCAGGTATTGTGTGTATA | 1711 |
| | i = 9 | | | TTGTAAAAAGGAGTTGTGTA | |
| | | | | GAGCAGGTGTTCGAGTGTTG | |
| | | | | TACAGCTCGCAGTGGAAAGC | |
| | | | | AGTGGAGACAGCC | |
| HPV82 | SD5-SA9 | 423 | 424 | GGAACTGCAGGCCCAAACAC | 1712 |
| | i = 11 | | | CGGAGGGCACCTCAGTGCAA | |
| | | | | CTAAAACTGCGTTTATAGTT | |
| | | | | CATTTAAAAGATGGCTTTGT | |
| | | | | GGCGTACTAATGACAGCAAA | |
| | | | | GTGTATTTACCACCTGCACC | |
| HPV82 | SD2-SA4 | 425 | 426 | CATCGGCAATGGACAGTGAA | 1713 |
| | i = 15 | | | GATCCAAATTTAATGTATTT | |
| | | | | ACATAGTAGAGTGACAGTAT | |
| | | | | TTCAATTTTTAAATGCATTT | |
| | | | | CCATTTGACCCCCAT | |
| HPV82 | SD2-SA6 | 427 | 428 | CATCGGCAATGGACAGTGAA | 1714 |
| | i = 17 | | | GTACCTACAGCACCCCGTCA | |
| | | | | CCCTCTACTACAACTG | |
| HPV82 | SD2-SA9 | 429 | 430 | CATCGGCAATGGACAGTGAA | 1715 |
| | i = 18 | | | GATGGCTTTGTGGCGTACTA | |
| | | | | ATGACAGCAAAGTGTATTTA | |
| | | | | CCACCTGCACC | |

TABLE 2B

| | | | For- | | Re- |
|---|---|---|---|---|---|
| | | | ward | | verse |
| | | Forward | pri- | Reverse | pri- |
| | | primer | mer | primer | mer |
| | | nucleic | SEQ | nucleic | SEQ |
| HPV | Splice | acid | ID | acid | ID |
| type | site | sequence | NO | sequence | NO |
| HPV16 | SD3 | GCGGGTATGG | 431 | TGGTGTTTGG | 432 |
| | | CAATACTGAA | | CATATAGTGT | |
| | | GT | | GTCTTT | |
| HPV16 | SD1 | CACAGAGCTG | 433 | CACATACAGC | 434 |
| | | CAAACAACTA | | ATATGGATTC | |
| | | TACAT | | CCATCTC | |
| HPV16 | SA4 | GGATGTAAAG | 435 | GTTTTCGTCA | 436 |
| | | CATAGACCAT | | AATGGAAACT | |
| | | TGGTACA | | CATTAGGA | |
| HPV16 | SA5 | CGGAAATCCA | 437 | TGACACACAT | 438 |
| | | GTGTATGAGC | | TTAAACGTTG | |
| | | TTAATGAT | | GCAAAG | |
| HPV16 | SA6 | CATGCGGGTG | 439 | AAGGCGACGG | 440 |
| | | GTCAGGTAA | | CTTTGGTAT | |
| HPV16 | SD5 | GCTCACACAA | 441 | CCAATGCCAT | 442 |
| | | AGGACGGATT | | GTAGACGACA | |
| | | AAC | | CT | |
| HPV16 | SA1 | GGAACAACAT | 443 | TGTCCAGATG | 444 |
| | | TAGAACAGCA | | TCTTTGCTTT | |
| | | ATACAACA | | TCTTCA | |
| HPV16 | SA2 | CGGTGGACCG | 445 | TCAGTTGTCT | 446 |
| | | GTCGATG | | CTGGTTGCAA | |
| | | | | ATCT | |
| HPV16 | SA9 | CCTATAGTTC | 447 | ATCCGTGCTT | 448 |
| | | CAGGGTCTCC | | ACAACCTTAG | |
| | | ACAA | | ATACTG | |
| HPV16 | SA3 | CTCAGAGGAG | 449 | CCATTAACAG | 450 |
| | | GAGGATGAAA | | GTCTTCCAAA | |
| | | TAGATG | | GTACGA | |
| HPV16 | SD2 | GGAATTGTGT | 451 | CATCCATTAC | 452 |
| | | GCCCCATCTG | | ATCCCGTACC | |
| | | T | | CT | |
| HPV18 | SD3 | TCAGATAGTG | 453 | CCGTTGTCTA | 454 |
| | | GCTATGGCTG | | TAGCCTCCGT | |
| | | TTCT | | | |
| HPV18 | SD1 | TTCACTGCAA | 455 | CTATACATTT | 456 |
| | | GACATAGAAA | | ATGGCATGCA | |
| | | TAACCTGT | | GCATGG | |
| HPV18 | SA4 | CTAAAATGTC | 457 | GTCATTTATT | 458 |
| | | CTCCAATACT | | TCATATACTG | |
| | | ACTAACCACA | | GATTGCCA | |
| | | A | | | |
| HPV18 | SD4 | GGATTGGACA | 459 | CCCATGCTAC | 460 |
| | | CTGCAAGACA | | ATAGGTCATA | |
| | | CA | | CAATTGTC | |
| HPV18 | SA8 | TGACGACACG | 461 | ACGTCTGGCC | 462 |
| | | GTATCCGCTA | | GTAGGTCT | |
| HPV18 | SD5 | CAGCTACACC | 463 | GTCGCTATGT | 464 |
| | | TACAGGCAAC | | TTTCGCAATC | |
| | | AA | | TGTA | |
| HPV18 | SD6 | CGAAAACATA | 465 | TTGTACACTA | 466 |
| | | GCGACCACTA | | TCTGGAATTG | |
| | | TAGAGAT | | CAACAGT | |
| HPV18 | SA1 | TCAGACTCTG | 467 | CCCAGCTATG | 468 |
| | | TGTATGGAGA | | TTGTGAAATC | |
| | | CACAT | | GT | |
| HPV18 | SA9 | TCCTAAGAAA | 469 | GTATTTACAA | 470 |
| | | CGTAAACGTG | | CTCTTGCCAC | |
| | | TTCCC | | AGAAGGA | |
| HPV18 | SA10 | GCATATTTTA | 471 | TCAGGTAACT | 472 |
| | | TCATGCTGGC | | GCACCCTAAA | |
| | | AGCTCTA | | TACTCTAT | |
| HPV18 | SA3 | CAGAGGAAGA | 473 | AGAAACAGCT | 474 |
| | | AAACGATGAA | | GCTGGAATGC | |
| | | ATAGATGG | | T | |
| HPV18 | SD2 | TGCATCCCAG | 475 | CTCGTCATCT | 476 |
| | | CAGTAAGCAA | | GATATTACAT | |
| | | | | CTCCTG | |
| | | | | TT | |
| HPV31 | SD3 | GCGGGTATGG | 477 | TGGAGTTTCA | 478 |
| | | CAATACTGAA | | TTCTCTCGTT | |
| | | GT | | CACTATG | |
| HPV31 | SD1 | CGGCATTGGA | 479 | TCTTAAACAT | 480 |
| | | AATACCCTAC | | TTTGTACACA | |
| | | GAT | | CTCCGTGT | |
| HPV31 | SA4 | CACTAGATGG | 481 | AATGTAAAAA | 482 |
| | | CAACCCTGTA | | CCACCAGTCT | |
| | | TCT | | GCTATGTA | |
| HPV31 | SA5 | CTGGTGGTTT | 483 | CGTTGAGAAA | 484 |
| | | TTACATTTCC | | GAGTCTCCAT | |
| | | AAATCCAT | | CGTTTT | |
| HPV31 | SA6 | CATGCGGGTG | 485 | GAATTCGATG | 486 |
| | | GTCAGGTAA | | TGGTGGTGTT | |
| | | | | GTTG | |
| HPV31 | SD5 | CAGCTGCATG | 487 | GCCATGTAGA | 488 |
| | | CACAAACCA | | TGACACTTGT | |
| | | | | TCATACAA | |
| HPV31 | SA1 | GGAACAACAT | 489 | TTTTCTTCTG | 490 |
| | | TAGAAAAATT | | GACACAACGG | |
| | | GACAAACAAA | | TCTT | |
| | | GG | | | |
| HPV31 | SA2 | GAAACGATTC | 491 | ACATAGTCTT | 492 |
| | | CACAACATAG | | GCAACGTAGG | |
| | | GAGGA | | TGTTT | |
| HPV31 | SA9 | GCCACAAGTG | 493 | TTTAGACACT | 494 |
| | | TCTATTTTTG | | GGGACAGGTG | |
| | | TTGATG | | GTA | |
| HPV31 | SA3 | CAGATGAGGA | 495 | CATTAACAGC | 496 |
| | | GGATGTCATA | | TCTTGCAATA | |
| | | GACAGT | | TGCGAATA | |
| HPV31 | SD2 | AATCGTGTGC | 497 | CCCCTGTCTG | 498 |
| | | CCCAACTGT | | TCTGTCAATT | |
| | | | | ACTG | |
| HPV33 | SD3 | GATGAGCTAG | 499 | CATCCCCCAC | 500 |
| | | AAGACAGCGG | | CCCACTAGAT | |
| | | ATATG | | | |
| HPV33 | SD1 | AGCATTGGAG | 501 | CGCAAACACA | 502 |
| | | ACAACTATAC | | GTTTACATAT | |
| | | ACAACATT | | TCCAAATG | |
| HPV33 | SA4 | TGTGAAACAT | 503 | CATACACTGG | 504 |
| | | AGGGCATTAG | | GTTACCATTT | |
| | | TGCAATTA | | TCATCAAA | |
| HPV33 | SA6 | GGATGCTGCA | 505 | GTGGTGGTCG | 506 |
| | | AAGTATTCTA | | GTTATCGTTG | |
| | | AAACACAA | | T | |
| HPV33 | SD5 | ACGTACTGCA | 507 | GCCAGGTGGA | 508 |
| | | ACTAACTGCA | | TGACATAGAA | |
| | | CAA | | CTATACA | |
| HPV33 | SA1 | ATTCTGTATA | 509 | TCGTTTGTTT | 510 |
| | | TGGAAATACA | | AAATCCACAT | |
| | | TTAGAACAAA | | GTCGTTTT | |
| | | CAG | | | |
| HPV33 | SA2 | CGATTTCATA | 511 | CATATTCCTT | 512 |
| | | ATATTTCGGG | | TAACGTTGGC | |
| | | TCGTTGG | | TTGTGT | |
| HPV33 | SA9 | TTGTTGTAGA | 513 | ATCAGTGCTG | 514 |
| | | CGGTGCTGAC | | ACAACTTTAG | |
| | | TTT | | ATACAGG | |
| HPV33 | SD2 | GTGCCCTACC | 515 | TTCTTCTCTC | 516 |
| | | TGTGCACAA | | TATGACTGCT | |
| | | | | TCTACCT | |
| HPV35 | SD3 | ATTATTTGAA | 517 | GCTACTAGAG | 518 |
| | | CTACCAGACA | | GTTATACTAT | |
| | | GCGGTT | | CCCCACT | |
| HPV35 | SD1 | CGAGGTAGAA | 519 | CATACTCCAT | 520 |
| | | GAAAGCATCC | | ATGGCTGGCC | |
| | | ATGAAAT | | TTC | |
| HPV35 | SA4 | CATTAGTGCA | 521 | TCATTGTGAA | 522 |
| | | ATTAAAATGC | | ATGTAAAGAC | |
| | | CCACCTT | | CACTACCC | |
| HPV35 | SA5 | GGAAACCCAG | 523 | GGAAAGCGTC | 524 |
| | | TGTATGGGCT | | TCCATCATTT | |
| | | TAAT | | TCTTTG | |
| HPV35 | SA6 | AAAATATATG | 525 | GCTTTGGTAT | 526 |
| | | GGAAGTGCAT | | GGGTCTCGGT | |
| | | GTGGGT | | | |
| HPV35 | SD5 | TCTACATCTG | 527 | CCATCTCCAT | 528 |
| | | ACTGCACAAA | | GTAGATGAAG | |
| | | CAAAGA | | CATCTTG | |
| HPV35 | SA1 | GGAGAAACGT | 529 | TCCACCGATG | 530 |
| | | TAGAAAAACA | | TTATGGAATC | |
| | | ATGCAACA | | GTTTT | |
| HPV35 | SA9 | GGGTGACTTT | 531 | CATCAGTGCT | 532 |
| | | TATTTACACC | | AACAACCTTA | |
| | | CTAGTT | | GACACT | |
| HPV35 | SA10 | CATCTACTAT | 533 | ACTCTGTATT | 534 |
| | | CATGCAGGCA | | GCAAACCAGA | |
| | | GTTCT | | TACCTTG | |
| HPV35 | SD2 | CGGCTGTTCA | 535 | CCCGTACGTC | 536 |
| | | CAGAGAGCAT | | TACTAACTAC | |
| | | AAT | | TGCTT | |
| HPV39 | SD3 | GGTGTATTCC | 537 | GTACACTGCC | 538 |
| | | GTGCCAGACA | | GCCATGTTC | |
| HPV39 | SD1 | CACCACCTTG | 539 | GATTGGCATG | 540 |
| | | CAGGACATTA | | CAGCTAGTGG | |
| | | CAATA | | | |
| HPV39 | SA4 | ATTAGATGGG | 541 | CTGTTTTGGT | 542 |
| | | TATGCAATAA | | CAAATGGAAA | |
| | | GTTTAGATAG | | TGCATTAG | |
| | | G | | | |
| HPV39 | SD5 | CACAGTAACA | 543 | CGTATCCAAT | 544 |
| | | GTACAGGCCA | | GCCAGGTACA | |
| | | CA | | TGAAA | |
| HPV39 | SA1 | CTCGGACTCG | 545 | CTGTCCTGTA | 546 |
| | | GTGTATGCAA | | TAGCTTCCTG | |
| | | | | CTATTTT | |
| HPV39 | SA9 | GCAATAACCA | 547 | AGTATTGACA | 548 |
| | | TTCAGGGTTC | | ACCTTCGCCA | |
| | | CAATT | | CA | |
| HPV39 | SA3 | CATGCAGTTA | 549 | TGCTGTAGTT | 550 |
| | | ATCACCAACA | | GTCGCAGAGT | |
| | | TCAACT | | ATC | |
| HPV39 | SD2 | CGTGGTGTGC | 551 | CACTGTGTCG | 552 |
| | | AACTGCAA | | CCTGTTTGTT | |
| | | | | TAT | |
| HPV45 | SD3 | TCAGATAGTG | 553 | ACTATCCCCA | 554 |
| | | GCTATGGCTG | | CCACTACTTT | |
| | | TTCT | | GTGTA | |
| HPV45 | SD1 | CTACAAGACG | 555 | AAGTCTATAC | 556 |
| | | TATCTATTGC | | ATTTATGGCA | |
| | | CTGTGT | | TGCAGCATA | |
| HPV45 | SA4 | CATTATTACA | 557 | GAAATGCATG | 558 |
| | | GCTAAAATGT | | TGGAAATGTA | |
| | | CCTCCAATCC | | AATACCGT | |
| HPV45 | SA8 | TGACGACACG | 559 | CCCACGGATG | 560 |
| | | GTATCCGCTA | | CGGTTTTG | |
| HPV45 | SD5 | TCCTGTGTTC | 561 | GGTCTGCATA | 562 |
| | | AAGTACAAGT | | TTTGCGTAGC | |
| | | AACAACAA | | CTATA | |
| HPV45 | SD6 | CGCAAATATG | 563 | CCCACCGAGA | 564 |
| | | CAGACCATTA | | TTTGTACACT | |
| | | CTCAGAA | | GTTA | |
| HPV45 | SA1 | AAACTCTGTA | 565 | CGTTTGTCCT | 566 |
| | | TATGGAGAGA | | TAAGGTGTCT | |
| | | CACTGGA | | ACGTTTT | |
| HPV15 | SA9 | GCACACAATA | 567 | GCTGACAACT | 568 |
| | | TTATTTATGG | | CTGGCCACA | |
| | | CCATGGTA | | | |
| HPV45 | SA3 | GGAGTTAGTC | 569 | TCAAAAACAG | 570 |
| | | ATGCACAACT | | CTGCTGTAGT | |
| | | ACCA | | GTTCT | |
| HPV45 | SD2 | AGCACCTTGT | 571 | CAATTGTTTC | 572 |
| | | CCTTTGTGTG | | TACAAAGAAC | |
| | | T | | CAGCCATT | |
| HPV51 | SD3 | CGGACAGCGG | 573 | TCTGTTGTTT | 574 |
| | | ATATGGCAAT | | CCACATCCAT | |
| | | A | | AACACT | |
| HPV51 | SD1 | CTGCATGAAT | 575 | GTAAACATTG | 576 |
| | | TATGTGAAGC | | TTTGCATACT | |
| | | TTTGAAC | | GCATATGGA | |
| HPV51 | SA4 | AGTATGTCCA | 577 | TCATTCAATG | 578 |
| | | CCATTACTAA | | TATACACAGC | |
| | | TAACGTCAAA | | ATTCCCAT | |
| | | C | | | |
| HPV51 | SA6 | GCACAACAGT | 579 | CCACGCAGGT | 580 |
| | | GGGAGGTCTA | | GGTAAGGG | |
| | | TATG | | | |
| HPV51 | SD5 | CTAACACTGG | 581 | ATGCCAGGTT | 582 |
| | | AGGGCACCAA | | GAGGATACGT | |
| | | A | | TTTTAT | |
| HPV51 | SA1 | GAGAGTATAG | 583 | TCCCGCTATT | 584 |
| | | ACGTTATAGC | | TCATGGAACC | |
| | | AGGTCTGT | | TTTT | |
| HPV51 | SA9 | GGCCCTATAC | 585 | CAATTCGAGA | 586 |
| | | ACATTTACTA | | CACAGGTGCA | |
| | | CGCAAA | | G | |
| HPV51 | SA3 | GCGTGACCAG | 587 | CATCTGCTGT | 588 |
| | | CTACCAGAAA | | ACAACGCGAA | |
| | | | | G | |
| HPV51 | SD2 | GGGCGAACTA | 589 | CTCATCATCC | 590 |
| | | AGCCTGGTTT | | GAAACATTAT | |
| | | | | CTCCTG | |
| | | | | T | |
| HPV52 | SD3 | CAAACCATGT | 591 | CCCCACCCCA | 592 |
| | | CACGTAGAAG | | CTTGATTGA | |
| | | ACAG | | | |
| HPV52 | SD1 | AGAATCGGTG | 593 | CACACGCCAT | 594 |
| | | CATGAAATAA | | ATGGATTATT | |
| | | GGCT | | GTCTCTA | |
| HPV52 | SA4 | CCTTAGTACA | 595 | GGGTTTTTGA | 596 |
| | | AATAAAATGC | | AATGAAACAC | |
| | | CCACCAT | | AACCAATC | |
| HPV52 | SA6 | GTAACAGGAG | 597 | GCGGAGGTCT | 598 |
| | | TATGGGAAGT | | TGGAGGTTT | |
| | | ACATGTG | | | |
| HPV52 | SD5 | TCACTGCAAC | 599 | TGCCAGGTAG | 600 |
| | | TGAGTGCACA | | ATGAAATTTG | |
| | | A | | AACATACA | |
| HPV52 | SA1 | GTATGGGAAA | 601 | CGCTTGTTTG | 602 |
| | | ACATTAGAAG | | CATTAACATG | |
| | | AGAGGGT | | TCTTTCT | |
| HPV52 | SA2 | GACATGTTAA | 603 | TCAGTTGTTT | 604 |
| | | TGCAAACAAG | | CAGGTTGCAG | |
| | | CGATTTC | | ATCTAATA | |
| HPV52 | SA9 | TTTTACTACG | 605 | TGCTTACAAC | 606 |
| | | TCGCAGGCGT | | CTTAGAGACA | |
| | | AA | | GGTACA | |
| HPV52 | SA10 | AAGCATCTAT | 607 | CCTGTATTGC | 608 |
| | | TATTATGCAG | | AGGCCAGACA | |
| | | GCAGTTCT | | | |
| HPV52 | SA3 | GATGAGGAGG | 609 | GCATTTGCTG | 610 |
| | | ATACAGATGG | | TAGAGTACGA | |
| | | TGTG | | AGGT | |
| HPV52 | SD2 | GCTGTTGGGC | 611 | TCCTCTGAAA | 612 |
| | | ACATTACAAG | | TGTTATCTCC | |
| | | TT | | TGTTTGTT | |
| HPV56 | SD3 | CAAGACAGCG | 613 | GGTACTGTTT | 614 |
| | | GGTATGGCAA | | TGTGAGCCTC | |
| | | TA | | CATTT | |
| HPV56 | SD1 | GCACCACTTG | 615 | ACAATAAACA | 616 |
| | | AGTGAGGTAT | | TACTCTGCAC | |
| | | TAGAA | | ACTGCATA | |
| HPV56 | SA5 | AGAATGTTAG | 617 | TTTTCTTTGT | 618 |
| | | TGTTTCAGTT | | CCTCGTCGTT | |
| | | TCAAAATCC | | ATCCAA | |
| HPV56 | SD5 | ACAACAACCA | 619 | TATTGTCTGT | 620 |
| | | CCCTGGTGAT | | ACTTGTCCAA | |
| | | AAG | | TGATATGT | |
| HPV56 | SA1 | TCAGTGTATG | 621 | CAATTGCTTT | 622 |
| | | GAGCTACACT | | TCCTCCGGAG | |
| | | AGAAAGT | | TTAA | |
| HPV56 | SA2 | TGCATTGTGA | 623 | ACGTCTTGCA | 624 |
| | | CAGAAAAAGA | | GCGTTGGTA | |
| | | CGATTTC | | | |
| HPV56 | SA9 | AGGGATCCTC | 625 | ACAACCTTTG | 626 |
| | | CTTTGCATTA | | AAACAGGTGT | |
| | | TGG | | TGGA | |
| HPV56 | SA10 | ATCATGCAGG | 627 | CAACCGTACC | 628 |
| | | CAGTTCACGA | | CTAAATACCC | |
| | | | | TATATTGA | |
| HPV56 | SA3 | ACAGCAAGCT | 629 | TGTACAACAC | 630 |
| | | AGACAAGCTA | | GCAGGTCCTC | |
| | | AACAA | | | |
| HPV56 | SD2 | GTTAACAGTA | 631 | TTCTACAATT | 632 |
| | | ACGTGCCCAC | | GCCTCTACTT | |
| | | TCT | | CAAACCAT | |
| HPV58 | SD3 | AAAATTATTG | 633 | CCCCACTAGA | 634 |
| | | AGCTAGAAGA | | CTCCGAGTCA | |
| | | CAGCGGAT | | TTTAA | |
| HPV58 | SD1 | GTCAGGCGTT | 635 | TCGTAAGCAC | 636 |
| | | GGAGACATCT | | ACTTTACATA | |
| | | | | CTGCAAA | |
| HPV58 | SA4 | ATTAGATGGT | 637 | TGCATCAAAT | 638 |
| | | AACGACATTT | | GGAAATGGAT | |
| | | CAATAGATGT | | TGTTAAATTC | |
| | | | | A | |
| HPV58 | SA6 | ACAATTATGG | 639 | CCCTGTGTAC | 640 |
| | | GAGGTACATG | | TTTCGTTGTT | |
| | | TGGGTA | | GGT | |
| HPV58 | SD5 | GAGGAGGACT | 641 | CCAATGCCAT | 642 |
| | | ACACAGTACA | | GTGGATGACA | |
| | | ACTAACT | | TATTACA | |
| HPV58 | SA1 | CGCTATATGG | 643 | CGACCCGAAA | 644 |
| | | AGACACATTA | | TATTATGAAA | |
| | | GAACAAACA | | CCTTTTGT | |
| HPV58 | SA9 | CTGATTTTAT | 645 | GCTTACAACC | 646 |
| | | GTTGCACCCT | | TTAGACACAG | |
| | | AGCTATTT | | GCA | |
| HPV58 | SD2 | TGCTTATGGG | 647 | CTGTTCTTCG | 648 |
| | | CACATGTACC | | TTCTATTACC | |
| | | ATT | | GCTTCTA | |
| HPV59 | SD3 | AAAGAAGGTT | 649 | GTCTATTTGA | 650 |
| | | AATAACAGTG | | CTGTCGCTAC | |
| | | CCAGACA | | AAACAC | |
| HPV59 | SD1 | GCATCAATTG | 651 | GCATTTCAGA | 652 |
| | | TGTGTTTTGC | | CACGCTGCAT | |
| | | AAAGG | | AC | |
| HPV59 | SA4 | AGATAGAAAG | 653 | TCTATTTTTG | 654 |
| | | CATAGGCACC | | TCAAATGGCA | |
| | | TAGTACAA | | ATTTGTTTGG | |
| | | | | A | |
| HPV59 | SD5 | TCCGTTTGCA | 655 | CCAATGCCAG | 656 |
| | | TCCAGGCAA | | GTAGAGGAAA | |
| | | | | TATTTTCA | |
| HPV59 | SA9 | CCTCGTAAAC | 657 | TGACATACTC | 658 |
| | | GTAAACGTGT | | ATCAGTGCTG | |
| | | TCC | | ACAAC | |
| HPV59 | SA10 | GTATGTCACC | 659 | GCCAAATTTA | 660 |
| | | CGTACCAGTA | | TTGGGATCAG | |
| | | TTTTCTAC | | GTAACTT | |
| HPV59 | SA3 | CAGATGGAGT | 661 | TGTAAGGCTC | 662 |
| | | TAATCATCCT | | GCAATCCGT | |
| | | TTGCTACT | | | |
| HPV59 | SD2 | ACTATCCTTT | 663 | CGTCATCTGA | 664 |
| | | GTGTGTCCTT | | AATTTTGTCA | |
| | | TGTGT | | CCTGTTTT | |
| HPV66 | SD3 | GAAGACAGCG | 665 | GATACCGAGT | 666 |
| | | GGTATGGCAA | | GCTCACTACA | |
| | | TA | | ATTACTG | |
| HPV66 | SD1 | CACCATCTGA | 667 | ACAATAAACA | 668 |
| | | GCGAGGTATT | | TACCCTACAT | |
| | | ACA | | ACTGCATATG | |
| | | | | G | |
| HPV66 | SA6 | GTGGGTGGTG | 669 | GGACAGTAAA | 670 |
| | | TAAAGTGTCA | | TACTCTCGGT | |
| | | TCA | | TTCCAT | |
| HPV66 | SD5 | GTATCAACAC | 671 | TCTGTACTTG | 672 |
| | | ACAAAGCCAC | | TCCAATGATA | |
| | | TGT | | TGTTGTTGT | |
| HPV66 | SA1 | GGGCAACATT | 673 | GAAATCGTCT | 674 |
| | | AGAAAGTATA | | TTTATGTTCA | |
| | | ACTAAAAAAC | | CAGTGCAA | |
| | | A | | | |
| HPV66 | SA9 | GCTACATTTG | 675 | ACAACCTTTG | 676 |
| | | CACTATGGCC | | AAACAGGTGT | |
| | | TGTA | | TGGA | |
| HPV66 | SA3 | ACAGCAAGCT | 677 | TGTACCACAC | 678 |
| | | AGACAAGCTG | | GTAGCTCCTC | |
| | | AA | | T | |
| HPV66 | SD2 | GTTAACAGTA | 679 | TTCTACAATT | 680 |
| | | ACGTGCCCAC | | GCTTCTACCT | |
| | | TCT | | GAAACCAT | |
| HPV68 | SD3 | AGACAACCGG | 681 | CACACTACTA | 682 |
| | | CGTATACAGT | | CAGTCCTCCC | |
| | | G | | GTAT | |
| HPV68 | SD1 | GACATTGGAC | 683 | GATTGGCATG | 684 |
| | | ACTACATTGC | | CAGCAAATGG | |
| | | ATGAC | | TA | |
| HPV68 | SA4 | CCTAATACAA | 685 | CTGTTTTGGT | 686 |
| | | ATAAAGTGTC | | CAAATGGAAA | |
| | | CACCAATGCT | | TGCATTAG | |
| HPV68 | SA1 | GGAATCGGTG | 687 | CTTCGTTTTG | 688 |
| | | TATGCAACTA | | TTGTTAGGTG | |
| | | CATTAGAA | | CCTTAG | |
| HPV68 | SA6 | CTAGTGGAAA | 689 | TCGCGGTGGT | 690 |
| | | ATGGGACGTG | | GTTCTGTAG | |
| | | CATTATA | | | |
| HPV68 | SD5 | AGTAGAAGTG | 691 | AAGCGTTATG | 692 |
| | | CAGGCCAAAA | | TTTTTGCAAC | |
| | | CAA | | CTATACC | |
| HPV68 | SA9 | TACAACCTTT | 693 | ATTGACAACC | 694 |
| | | GCCATAACTA | | TTCGCCACTG | |
| | | TATATGGT | | A | |
| HPV68 | SA3 | CCACCAACAT | 695 | CTGTTGTAGT | 696 |
| | | CTACTACTAG | | GTCCGCAGGT | |
| | | CCAGA | | T | |
| HPV68 | SD2 | TCCGTGGTGT | 697 | GACTGTGTCA | 698 |
| | | GCAACTGAA | | CCTGTTTGTT | |
| | | | | TATCTACT | |
| HPV73 | SD3 | AAACGAAGAC | 699 | GACACAATTT | 700 |
| | | TGTTTGAGGA | | GGTTGCCTTC | |
| | | GCA | | TTCATTAA | |
| HPV73 | SD1 | AGCGTTATGT | 701 | AAAATTTTAA | 702 |
| | | GACGAAGTGA | | ACACGGTTGA | |
| | | ATATTTCT | | CATACAC | |
| HPV73 | SA4 | CAAGTTAAAT | 703 | GGGTTCCCAT | 704 |
| | | GCCCTCCATT | | TACTGTCAAA | |
| | | ACTGATAAC | | TGGA | |
| HPV73 | SA6 | GGGTAAAAGG | 705 | TGGTGTTGGT | 706 |
| | | CATATGGGAA | | GGTTGTGGT | |
| | | GTACAT | | | |
| HPV73 | SD5 | ACCTACATCC | 707 | GTCCAATGCC | 708 |
| | | CACCACAGAG | | ATGTTGTTGT | |
| | | T | | TACA | |
| HPV73 | SA1 | AGACAATCAG | 709 | CTGTTCTGCT | 710 |
| | | TATATGGCAC | | ATTTGATGAA | |
| | | TACGTTAGA | | ACCGTTTT | |
| HPV73 | SA9 | TGGGTCAGGT | 711 | GCTTACAACC | 712 |
| | | TTTATATTAC | | TTAGACACAG | |
| | | ACCCTAGT | | ACACA | |
| HPV73 | SA10 | TGCAGGTAGC | 713 | ACGAAGCCTA | 714 |
| | | ACACGTTTGT | | AACACCCTGT | |
| | | | | ATTG | |
| HPV73 | SA3 | ACTCAGAGGA | 715 | CCTAGTGTAC | 716 |
| | | TGAGGATGAA | | CCATAAGCAA | |
| | | ACAGA | | CTCTTCTA | |
| HPV73 | SD2 | TGCTTATGGG | 717 | TGGAATTGGA | 718 |
| | | TACACTAGGT | | TCCCCTGTTT | |
| | | ATTGTGT | | TTCTTT | |
| HPV82 | SD3 | CCGGACAGTG | 719 | GGTCTATCTC | 720 |
| | | GATATGGCAA | | TGTACTTCTG | |
| | | TA | | TCGCT | |
| HPV82 | SD1 | CCTGCAATAC | 721 | CATGCTGCAT | 722 |
| | | GTCTATGCAC | | ATGGCGTATT | |
| | | AAT | | GTC | |
| HPV82 | SA4 | ACACAGAAGC | 723 | CATCATTTAG | 724 |
| | | CTGCTGCAAA | | TGCATATACA | |
| | | | | GGATTCCC | |
| HPV82 | SA6 | GGGCACAACA | 725 | GGGTGTTCGA | 726 |
| | | ATGGGAGGTA | | TAGCTGTTCA | |
| | | | | A | |
| HPV82 | SD5 | TGCGACCACC | 727 | CAATGCCAGG | 728 |
| | | AAATACACTG | | TAGATGACAC | |
| | | T | | TTCTTTAA | |
| HPV82 | SA1 | GTAGGTCTGT | 729 | TTTTTTGTCG | 730 |
| | | GTATGGTGCT | | TCCACCACCT | |
| | | ACATT | | TTTG | |
| HPV82 | SA9 | GGGATTACTA | 731 | GTGTTGACAA | 732 |
| | | CTTTGTGGCC | | TGCGTGACAC | |
| | | GTATA | | T | |
| HPV82 | SA3 | GGAGGATGAA | 733 | CCAGTAACAT | 734 |
| | | GTAGATAATA | | TTGCTGAAAT | |
| | | TGCGTGAC | | ATGCGAA | |
| HPV82 | SD2 | CGTGGTGTGC | 735 | TTGTCAACTA | 736 |
| | | GACCAACTAA | | CTGCCTCCAC | |
| | | | | ATAAAA | |

TABLE 2Bbis

| | | For- | Re- | | |
|---|---|---|---|---|---|
| | | ward | verse | | |
| | | pri- | pri- | | Ampli- |
| | | mer | mer | | con |
| | | SEQ | SEQ | Amplicon | SEQ |
| HPV | Splice | ID | ID | nucleic acid | ID |
| type | site | NO | NO | sequence | NO |
| HPV16 | SD3 | 431 | 432 | GGAAACTCAGCAGATGTTAC | 1716 |
| | | | | AGGTAGAAGGGCGCCATGAG | |
| | | | | ACTGAAACACCATGTAGTCA | |
| | | | | GTATAGTGGTGGAAGTGGGG | |
| | | | | GTGGTTGCAGTCAGTACAGT | |
| | | | | AGTGGAAGTGGGGGAGAGGG | |
| | | | | TGTTAGTG | |
| HPV16 | SD1 | 433 | 434 | GATATAATATTAGAATGTGT | 1717 |
| | | | | GTACTGCAAGCAACAGTTAC | |
| | | | | TGCGACGTGAGGTATATGAC | |
| | | | | TTTGCTTTTCGGGATTTATG | |
| | | | | CATAGTATATA | |
| HPV16 | SA4 | 435 | 436 | ACTAAAATGCCCTCCATTAT | 1718 |
| | | | | TAATTACATCTAACATTAAT | |
| | | | | GCTGGTACAGATTCTAGGTG | |
| | | | | GCCTTATTTACATAATAGAT | |
| | | | | TGGTGGTGTTTACATT | |
| HPV16 | SA5 | 437 | 438 | AAGAACTGGAAATCCTTTTT | 1719 |
| | | | | CTCAAGGACGTGGTCCAGAT | |
| | | | | TAAGTTTGCACGAGGACGAG | |
| | | | | GACAAGGAAAACGATGGAGA | |
| | | | | CT | |
| HPV16 | SA6 | 439 | 440 | TATTATGTCCTACATCTGTG | 1720 |
| | | | | TTTAGCAGCAACGAAGTATC | |
| | | | | CTCTCCTGAAATTATTAGGC | |
| | | | | AGCACTTGGCCAACCACCCC | |
| | | | | GCCGCGACCC | |
| HPV16 | SD5 | 441 | 442 | TGTAATAGTAACACTACACC | 1721 |
| | | | | CATAGTACATTTAAAAGGTG | |
| | | | | ATGCTAATACTTTAAAATGT | |
| | | | | TTAAGATATAGATTTAAAAA | |
| | | | | GCATTGTACATTGTATACTG | |
| | | | | C | |
| HPV16 | SA1 | 443 | 444 | AACCGTTGTGTGATTTGTTA | 1722 |
| | | | | ATTAGGTGTATTAACTGTCA | |
| | | | | AAAGCCACTGTGTCC | |
| HPV16 | SA2 | 445 | 446 | TATGTCTTGTTGCAGATCAT | 1723 |
| | | | | CAAGAACACGTAGAGAAACC | |
| | | | | CAGCTGTAATCATGCATGGA | |
| | | | | GATACACCTACATTGCATGA | |
| | | | | ATATATGTT | |
| HPV16 | SA9 | 447 | 448 | TATACAATTATTGCTGATGC | 1724 |
| | | | | AGGTGACTTTTATTTACATC | |
| | | | | CTAGTTATTACATGTTACGA | |
| | | | | AAACGACGTAAACGTTTACC | |
| | | | | ATATTTTTTTTCAGATGTCT | |
| | | | | CTTTGGCTGCCTAGTGAGGC | |
| | | | | CACTGTCTACTTGCCTCCTG | |
| | | | | TCC | |
| HPV16 | SA3 | 449 | 450 | GTCCAGCTGGACAAGCAGAA | 1725 |
| | | | | CCGGACAGAGCCCATTACAA | |
| | | | | TATTGTAA | |
| | | | | CCTTTTGTTGCAAGTGTGAC | |
| | | | | TCTACGCTTCGGTTGTGCGT | |
| | | | | ACAAAGCACACACGTAGACA | |
| | | | | T | |
| HPV16 | SD2 | 451 | 452 | TCTCAGAAACCATAATCTAC | 1726 |
| | | | | CATGGCTGATCCTGCAGGTA | |
| | | | | CCAATGGGGAAG | |
| HPV18 | SD3 | 453 | 454 | GAAGTGGAAGCAACACAGAT | 1727 |
| | | | | TCAGGTAACTACAAATGGCG | |
| | | | | AACATGGCGGCAATGTATGT | |
| | | | | AGTGGCGGCAGT | |
| HPV18 | SD1 | 455 | 456 | GTATATTGCAAGACAGTATT | 1728 |
| | | | | GGAACTTACAGAGGTATTTG | |
| | | | | AATTTGCATTTAAAGATTTA | |
| | | | | TTTGTGGTGTATAGAGACAG | |
| | | | | TATACC | |
| HPV18 | SA4 | 457 | 458 | ATATACATCCAGCAAAGGAT | 1729 |
| | | | | AATAGATGGCCATATTTAGA | |
| | | | | AAGTAGAATAACAGTATTTG | |
| | | | | AATTTCCAAATGCATTTCCA | |
| | | | | TTTGATAAAAA | |
| HPV18 | SD4 | 459 | 460 | TGCGAGGAACTATGGAATAC | 1730 |
| | | | | AGAACCTACTCACTGCTTTA | |
| | | | | AAAAAGGTGGCCAAACAGTA | |
| | | | | CAAGTATATTTTGATGGCAA | |
| | | | | CAAA | |
| HPV18 | SA8 | 461 | 462 | CTCAGCTTGTTAAACAGCTA | 1731 |
| | | | | CAGCACACCCCCTCACCGTA | |
| | | | | TTCCAGCACCGTGTCCGTGG | |
| | | | | GCACCGCAA | |
| HPV18 | SD5 | 463 | 464 | CAAAAGACGGAAACTCTGTA | 1732 |
| | | | | GTGGTAACACTACGCCTATA | |
| | | | | ATACATTTAAAAGGTGACAG | |
| | | | | AAACAGTTTAAAATGTTTAC | |
| | | | | GG | |
| HPV18 | SD6 | 465 | 466 | ATATCATCCACCTGGCATTG | 1733 |
| | | | | GACAGGTGCAGGCAATGAAA | |
| | | | | AAACAGGAATACTGACTGTA | |
| | | | | ACATACCATAGTGAAACACA | |
| | | | | AAGAACAAAATTTTTAAAT | |
| HPV18 | SA1 | 467 | 468 | TGGAAAAACTAACTAACACT | 1734 |
| | | | | GGGTTATACAATTTATTAAT | |
| | | | | AAGGTGCCTGCGGTGCCAGA | |
| | | | | AACCGTTGAATCCAGCAGAA | |
| | | | | AAACTTAGACACCTTAATGA | |
| | | | | AAAACG | |
| HPV18 | SA9 | 469 | 470 | TATTTTTTTGCAGATGGCTT | 1735 |
| | | | | TGTGGCGGCCTAGTGACAAT | |
| | | | | ACCGTATATCTTCCACC | |
| HPV18 | SA10 | 471 | 472 | GATTATTAACTGTTGGTAAT | 1736 |
| | | | | CCATATTTTAGGGTTCCTGC | |
| | | | | AGGTGGTGGCAATAAGCAGG | |
| | | | | ATATTCCTAAGGTTTCTGCA | |
| | | | | TACCAAT | |
| HPV18 | SA3 | 473 | 474 | AGTTAATCATCAACATTTAC | 1737 |
| | | | | CAGCCCGACGAGCCGAACCA | |
| | | | | CAACGTCACACAATGTTGTG | |
| | | | | TATGTGTTGTAAGTGTGAAG | |
| | | | | CCAGAATTGAGCTAGTAGTA | |
| | | | | GAAAGCTCAGCAGACGACCT | |
| | | | | TCG | |
| HPV18 | SD2 | 475 | 476 | CAATGGCTGATCCAGAAGGT | 1738 |
| | | | | ACAGACGGGGAGGGCACGGG | |
| | | | | TTGTAACG | |
| | | | | GCTGGTTTTATGTACAAGCT | |
| | | | | ATTGTAGACAAAAA | |
| HPV31 | SD3 | 477 | 478 | GGAAACGCAGCAGATGGTAC | 1739 |
| | | | | AGGTAGAGGAGCAACAAACA | |
| | | | | ACATTAAGTTGTAATGGTAG | |
| | | | | TGACGGGACA | |
| HPV31 | SD1 | 479 | 480 | GAACTAAGATTGAATTGTGT | 1740 |
| | | | | CTACTGCAAAGGTCAGTTAA | |
| | | | | CAGAAACAGAGGTATTAGAT | |
| | | | | TTTGCATTTACAGATTTAAC | |
| | | | | AATAGTATATAGGGACGACA | |
| | | | | CACC | |
| HPV31 | SA4 | 481 | 482 | ATAGATGTAAAGCATAAAGC | 1741 |
| | | | | TTTAATGCAGTTAAAATGTC | |
| | | | | CTCCTTTATTGATTACATCT | |
| | | | | AATATAAATGCAGGTAAGGA | |
| | | | | TGACAGATGGCCATACC | |
| HPV31 | SA5 | 483 | 484 | TTCCATTTGACAAAAACGGA | 1742 |
| | | | | AATCCAGTATATGAATTAAG | |
| | | | | TGATAAAAACTGGAAATCCT | |
| | | | | TTTTCTCAAGGACGTGGTGC | |
| | | | | AGATTAAATTTGCACGAGGA | |
| | | | | AGAGGACAAAG | |
| HPV31 | SA6 | 485 | 486 | TTCTTTTTCCTGAATCTGTA | 1743 |
| | | | | TTTAGCAGTGACGAAATATC | |
| | | | | CTTTGCTGGGATTGTTACAA | |
| | | | | AGCTACCAACAGC | |
| HPV31 | SD5 | 487 | 488 | AACAAGGGCTGTCAGTTGTC | 1744 |
| | | | | CTGCAACTACACCTATAATA | |
| | | | | CACTTAAAAGGTGATGCAAA | |
| | | | | TATATTAAAATGTTTAAGAT | |
| | | | | ATAGGCTGTCAAAATATAAA | |
| | | | | CAA | |
| HPV31 | SA1 | 489 | 490 | TATATGTGATTTGTTAATTA | 1745 |
| | | | | GGTGTATAACGTGTCA | |
| HPV31 | SA2 | 491 | 492 | AGGTGGACAGGACGTTGCAT | 1746 |
| | | | | AGCATGTTGGAGAAGACCTC | |
| | | | | GTACTGAAACCCAAGTGTAA | |
| | | | | ACATGCGTGGAG | |
| HPV31 | SA9 | 493 | 494 | GGGGTGATTTTTATTTGCAC | 1747 |
| | | | | CCTAGTTATTATATGTTAAA | |
| | | | | ACGTCGACGTGCTACTGTCT | |
| | | | | ACT | |
| HPV31 | SA3 | 495 | 496 | CCAGCTGGACAAGCAGAACC | 1748 |
| | | | | GGACACATCCAATTACAATA | |
| | | | | TCGTTACCTTTTGTTGTCAG | |
| | | | | TGTAAGTCTACACTTCGTTT | |
| | | | | GTGTGTACAGAGCACACAAG | |
| | | | | TAGA | |
| HPV31 | SD2 | 497 | 498 | TCTACTAGACTGTAACTACA | 1749 |
| | | | | ATGGCTGATCCAGCAGGTAC | |
| | | | | AGATGGGGAGGGGACGGGAT | |
| | | | | GCAATGGTTGGTTTTATGTA | |
| | | | | GAAG | |
| HPV33 | SD3 | 499 | 500 | GCAATACTGAAGTGGAAACT | 1750 |
| | | | | CAGCAGATGGTACAACAGGT | |
| | | | | AGAAAGTCAAAATGGCGACA | |
| | | | | CAAACTTAAATGACTTAGA | |
| HPV33 | SD1 | 501 | 502 | GAACTACAGTGCGTGGAATG | 1751 |
| | | | | CAAAAAACCTTTGCAACGAT | |
| | | | | CTGAGGTA | |
| | | | | TATGATTTTGCATTTGCAGA | |
| | | | | TTTAACAGTTGTATATAGAG | |
| | | | | AGGGAAATC | |
| HPV33 | SA4 | 503 | 504 | AAATGTCCACCACTGCTTCT | 1752 |
| | | | | TACCTCAAATACAAATGCAG | |
| | | | | GCACAGACTCTAGATGGCCA | |
| | | | | TATTTACATAGTAGATTAAC | |
| | | | | AGTATTTGAATTTAAAAATC | |
| | | | | CATTCCCA | |
| HPV33 | SA6 | 505 | 506 | ATGTGGGAAGTACATGTGGG | 1753 |
| | | | | TGGTCAGGTAATTGTTTGTC | |
| | | | | CTACGTCTATATCTAGCAAC | |
| | | | | CAAATATCCACTACTGAAAC | |
| | | | | TGCTGACATACAGACAG | |
| HPV33 | SD5 | 507 | 508 | ACAAGCAGCGGACTGTGTGT | 1754 |
| | | | | AGTTCTAACGTTGCACCTAT | |
| | | | | AGTGCATTTAAAAGGTGAAT | |
| | | | | CAAATAGTTTAAAATGTTTA | |
| | | | | AGATACAGATTAAAACCTTA | |
| | | | | TAAAGAGT | |
| HPV33 | SA1 | 509 | 510 | TTAAAAAACCTTTAAATGAA | 1755 |
| | | | | ATATTAATTAGGTGTATTAT | |
| | | | | ATGTCAAAGACCTTTGTGTC | |
| | | | | CTCAAGAAAA | |
| HPV33 | SA2 | 511 | 512 | GCAGGGCGCTGTGCGGCGTG | 1756 |
| | | | | TTGGAGGTCCCGACGTAGAG | |
| | | | | AAACTGCACTGTGACGTGTA | |
| | | | | AAAACGCCATGAGAGG | |
| HPV33 | SA9 | 513 | 514 | GTTTTACATCCTAGTTATTT | 1757 |
| | | | | TATTTTACGTCGCAGGCGTA | |
| | | | | AACGTTTTCCATATTTTTTT | |
| | | | | ACAGATGTCCGTGTGGCGGC | |
| | | | | CTAGTGAGGCCACAGTGTAC | |
| | | | | CTGCCTCCTGTA | |
| HPV33 | SD2 | 515 | 516 | CAATAAACATCATCTACAAT | 1758 |
| | | | | GGCCGATCCTGAAGGTACAA | |
| | | | | ATGGGGCTGGGATGGGGTGT | |
| | | | | ACTGGTTGGTTTG | |
| HPV35 | SD3 | 517 | 518 | ATGGCAATTCTGAAGTGGAA | 1759 |
| | | | | ATACAGCAGATACAACAGGT | |
| | | | | AGAGGGGCATGATACAGTTG | |
| | | | | AACAATGTAGTATGGGC | |
| HPV35 | SD1 | 519 | 520 | TTGTTTGAATTGTGTATACT | 1760 |
| | | | | GCAAACAAGAATTACAGCGG | |
| | | | | AGTGAGGTATATGACTTTGC | |
| | | | | ATGCTATGATTTGTGTATAG | |
| | | | | TATATAGA | |
| HPV35 | SA4 | 521 | 522 | TACTTATTACATCAAATATA | 1761 |
| | | | | AATGCAGGCAAAGATGACAG | |
| | | | | GTGGCCATACTTACATAGCA | |
| HPV35 | SA5 | 523 | 524 | GATAAAAACTGGAAATCCTT | 1762 |
| | | | | TTTCTCAAGGACGTGGTGCA | |
| | | | | GATTAAATTTGCACGAGGAA | |
| | | | | GAGGA | |
| HPV35 | SA6 | 525 | 526 | GGTCAGGTAATTGTTTGTCC | 1763 |
| | | | | TGAATCTGTATTTAGCAGCA | |
| | | | | CAGAACTATCCACTGCTGAA | |
| | | | | ATTGCTACACAGCTACACGC | |
| | | | | CTACAACACC | |
| HPV35 | SD5 | 527 | 528 | CCGGTGTGGTAGTTGTAGTA | 1764 |
| | | | | CAACTACACCTATAGTACAT | |
| | | | | TTAAAAGGTGATGCAAATAC | |
| | | | | ATTAAAGTGTTTAAGATATA | |
| | | | | GATTGGGTAAATATAAA | |
| HPV35 | SA1 | 529 | 530 | AACAGTTATGTCATTTATTA | 1765 |
| | | | | ATTAGGTGTATTACATGTCA | |
| | | | | AAAACCGCT | |
| | | | | GTGTCCAGTTGAAAAGCAAA | |
| | | | | GACATTTAGAAGAAAA | |
| HPV35 | SA9 | 531 | 532 | GTCTCTGTGGCGGTCTAACG | 1766 |
| | | | | AAGCCACTGTCTACCTGCCT | |
| | | | | CCAGTGTC | |
| HPV35 | SA10 | 533 | 534 | AGGCTATTAGCTGTGGGTCA | 1767 |
| | | | | CCCATACTATGCTATTAAAA | |
| | | | | AACAAGATT | |
| | | | | CTAATAAAATAGCAGTACC | |
| HPV35 | SD2 | 535 | 536 | CTACAATGGCTGATCCTGCA | 1768 |
| | | | | GGTACAGATGAAGGGGAGGG | |
| | | | | GACGGGAT | |
| | | | | GTAATGGATGGTTTTTTGTA | |
| | | | | G | |
| HPV39 | SD3 | 537 | 538 | GCGGATATGGCAATATGGAA | 1769 |
| | | | | GTGGAAACAGCTGAAGTGGA | |
| | | | | GGAGGTAA | |
| | | | | CTGTAGCAACTAATACAAAT | |
| | | | | GGGGATGCTGAAGGG | |
| HPV39 | SD1 | 539 | 540 | GCCTGTGTCTATTGCAGACG | 1770 |
| | | | | ACCACTACAGCAAACCGAGG | |
| | | | | TATATGAATTTGCATTTAG | |
| | | | | TGATTTATATGTAGTATAT | |
| | | | | AGGGACGGGGAA | |
| HPV39 | SA4 | 541 | 542 | AAATATAAAAGTTTACTACA | 1771 |
| | | | | AATGAAATGTCCACCATTAT | |
| | | | | TAATAACCT | |
| | | | | CCAATACCAATCCTGTGGAA | |
| | | | | GACGATAGGTGGCCATATTT | |
| | | | | ACGTAGTAG | |
| | | | | GCTAACAGTGTTTAAATTTC | |
| HPV39 | SD5 | 543 | 544 | ACACAAGACGGTACCTCAGT | 1772 |
| | | | | TGTGGTAACACTACGCCTAT | |
| | | | | AATACATTT | |
| | | | | AAAAGGTGACAAAAATGGTT | |
| | | | | TAAAATGTTTAAGATATAGA | |
| | | | | CTACAAAA | |
| | | | | ATATGACACATTGTTTGAAA | |
| | | | | ATA | |
| HPV39 | SA1 | 545 | 546 | CTACATTAGAAAATATAACT | 1773 |
| | | | | AATACAAAGTTATATAATTT | |
| | | | | ATTAATAAG | |
| | | | | GTGCATGTGTTGTCTGAAAC | |
| | | | | CGCTGTGTCCAGCAGAAAAA | |
| | | | | TTAAGACAC | |
| | | | | CTAAATAGCAAACGAAGATT | |
| | | | | TCAT | |
| HPV39 | SA9 | 547 | 548 | ATTATTTGTTGCCATTATTG | 1774 |
| | | | | TATTTTTTCCTAAAAAAACG | |
| | | | | TAAACGTATT | |
| | | | | ATTTGCCTCCACCTTC | |
| HPV39 | SA3 | 549 | 550 | ACTAGCCAGACGGGATGAAC | 1775 |
| | | | | CACAGCGTCACACAATACAG | |
| | | | | TGTTCGTGTTGTAAGTGTAA | |
| | | | | CAACACACTGCAGCTGGTAG | |
| | | | | TAGAAGCCTCACGG | |
| HPV39 | SD2 | 551 | 552 | ACCAGTAACCTGCTATGGCC | 1776 |
| | | | | AATCGTGAAGGTACAGACGG | |
| | | | | GGATGGGTCGGGATGTAACG | |
| | | | | GATGGTTTCTAGTACAGGCA | |
| | | | | ATAGTAG | |
| HPV45 | SD3 | 553 | 554 | GAAGTGGAAGCTGCAGAGAC | 1777 |
| | | | | TCAGGTAACTGTAAACACTA | |
| | | | | ATGCGGAA | |
| | | | | AATGGCGGCAGTGTACATAG | |
| HPV45 | SD1 | 555 | 556 | ATATTGCAAAGCAACATTGG | 1778 |
| | | | | AACGCACAGAGGTATATCAA | |
| | | | | TTTGCTTTTAAAGATTTATG | |
| | | | | TATAGTGTATAGAGACTGTA | |
| | | | | TAGCA | |
| HPV45 | SA4 | 557 | 558 | TATTAACATCCAATATTGAT | 1779 |
| | | | | CCAGCAAAAGATAATAAATG | |
| | | | | GCCATATTTAGAAAGTAGGG | |
| | | | | TG | |
| HPV45 | SA8 | 559 | 560 | CTCAGATTGTTAGACAGCTA | 1780 |
| | | | | CAACACGCCTCCACGTCGAC | |
| | | | | CCC | |
| HPV45 | SD5 | 561 | 562 | AAGAAGGAAAGTGTGTAGTG | 1781 |
| | | | | GTAACACTACGCCTATAATA | |
| | | | | CACTTAAAAGGTGACAAAAA | |
| | | | | CAGTTTGAAATGTTTAAGA | |
| HPV45 | SD6 | 563 | 564 | ATATCCTCCACCTGGCATTG | 1782 |
| | | | | GACAGGTTGTAATAAAAACA | |
| | | | | CTGGTATATTAACTGTAACA | |
| | | | | TATAATAGTGAGGTACAAAG | |
| | | | | AAATACCTTTTTGGATGTAG | |
| | | | | TTACTATTCC | |
| HPV45 | SA1 | 565 | 566 | AAAAATAACTAATACAGAGT | 1783 |
| | | | | TGTATAATTTGTTAATAAGG | |
| | | | | TGCCTGCGGTGCCAGAAACC | |
| | | | | ATTGAACCCAGCAGA | |
| HPV45 | SA9 | 567 | 568 | TTATTATTTTCCTAAAAAAC | 1784 |
| | | | | GTAAACGTATTCCCTATTTT | |
| | | | | TTTGCAGATGGCTTTGTGGC | |
| | | | | GGCCTAGTGACAGTACGGTA | |
| | | | | TATCTTCCACCACCTTC | |
| HPV45 | SA3 | 569 | 570 | GCCCGACGAGCCGAACCACA | 1785 |
| | | | | GCGTCACAAAATTTTGTGTG | |
| | | | | TATGTTGTAAGTGTGACGGC | |
| | | | | AGAATTGAGCTTACAGTAGA | |
| | | | | GAGCTCGGCAGAGGACCTT | |
| HPV45 | SD2 | 571 | 572 | CCGTGGTGTGCAACTAACCA | 1786 |
| | | | | ATAATCTACAATGGCGGATC | |
| | | | | CAGAAGGTACCGACGGGGAG | |
| | | | | GGAACGGGGTGT | |
| HPV51 | SD3 | 573 | 574 | CACAAGTGGAAACTGTGGAA | 1787 |
| | | | | GCAACGTTGCAGGTAGATGG | |
| | | | | GCAACATGGCGGTTCACAGA | |
| | | | | ACAGTGTGTGTAGTAGCGGG | |
| | | | | GGGGGC | |
| HPV51 | SD1 | 575 | 576 | CiTTTCTATGCACAATATAC | 1788 |
| | | | | AGGTAGTGTGTGTGTATTGT | |
| | | | | AAAAAGGAATTATGTAGAGC | |
| | | | | AGATGTATATAATGTAGCAT | |
| | | | | TTACTGAAATTAAGATTGTA | |
| | | | | TATAGGGATAATAA | |
| HPV51 | SA4 | 577 | 578 | ATAAATCCACAAGAGGATGC | 1789 |
| | | | | AAACCTAATGTATTTACATA | |
| | | | | CAAGGGTAACAGTATTAAAG | |
| | | | | TTTTTAAATACATTTCCATT | |
| | | | | TGATAACA | |
| HPV51 | SA6 | 579 | 580 | TATGGTACTGTAATAACATG | 1790 |
| | | | | TCCTGAATATGTATCTAGTA | |
| | | | | CCTGCAGCGACGCGTTATCC | |
| | | | | ACTACTACAACTGTTGAACA | |
| | | | | ACTATCAAACACCCCAACGA | |
| | | | | CCAATC | |
| HPV51 | SD5 | 581 | 582 | GTGCAACTCAGACTGCGTTT | 1791 |
| | | | | ATAGTGCATTTAAAAGGTGA | |
| | | | | TACAAATTGTTTAAAATGTT | |
| | | | | TTAGATACAGATTTACAAAA | |
| | | | | CACAAAGGGTTAT | |
| HPV51 | SA1 | 583 | 584 | GTATGGTACTACATTAGAGG | 1792 |
| | | | | CAATTACTAAAAAAAGCTTA | |
| | | | | TATGATTTATCGATAAGGTG | |
| | | | | TCATAGATGTCAAAGACCAC | |
| | | | | TTGGGCCTGAAGAAAAGCAA | |
| | | | | AAATTGGTGGACGAAAAA | |
| HPV51 | SA9 | 585 | 586 | CGCCGTAAACGTATACCCTA | 1793 |
| | | | | TTTTTTTACAGATGGCATTG | |
| | | | | TGGCGCACTAATGACAGCAA | |
| | | | | GGTGTATTTGCCAC | |
| HPV51 | SA3 | 587 | 588 | GACGGGCTGGACAGGCTACG | 1794 |
| | | | | TGTTACAGAATTGAAGCTCC | |
| | | | | GTGTTGCAGGTGTTCAAGTG | |
| | | | | TAGTACAACTGGCAGTGGAA | |
| | | | | AGCAGTGGAGACACC | |
| HPV51 | SD2 | 589 | 590 | GCCCGTGTTGTGCGAACAAC | 1795 |
| | | | | TAGCAACGGCGATGGACTGT | |
| | | | | GAAGGTACAGAGGATGAGGG | |
| | | | | GGCGGGGTGTAATGGGTGGT | |
| | | | | TTTTTGTTGAAGCAATAGTA | |
| | | | | GAAAAAAAA | |
| HPV52 | SD3 | 591 | 592 | CGGCTATGGCAATAGTGAAG | 1796 |
| | | | | TGGAAGCGCAGCAGATGGCA | |
| | | | | GACCAGGTAGACGGGCAAAA | |
| | | | | TGGCGACTGGCAAAGTAACA | |
| | | | | GTAG | |
| HPV52 | SD1 | 593 | 594 | GCAGTGTGTGCAGTGCAAAA | 1797 |
| | | | | AAGAGCTACAACGAAGAGAG | |
| | | | | GTATACAAGTTTCTATTTAC | |
| | | | | AGATTTACGAATAGTATA | |
| HPV52 | SA4 | 595 | 596 | TAATTTTAACAACAAATACA | 1798 |
| | | | | AATGCAGGAACAGATCCTAG | |
| | | | | GTGGCCATATTTACATAGTA | |
| HPV52 | SA6 | 597 | 598 | GGTGGTCAGGTAATTGTTTG | 1799 |
| | | | | TCCTGCATCTGTATCTAGTA | |
| | | | | ACGAAGTATCCACTACTGAA | |
| | | | | ACTGCTGTCCACCTATGCAC | |
| | | | | CG | |
| HPV52 | SD5 | 599 | 600 | ACAAAGGACGGGTTGCACAT | 1800 |
| | | | | ACAACTTGTACTGCACCTAT | |
| | | | | AATACACCTAAAAGGTGATC | |
| | | | | CTAATAGTTTAAAATGTTTA | |
| | | | | AGATATAGGGTAAAAACACA | |
| | | | | TAAAAGTT | |
| HPV52 | SA1 | 601 | 602 | AAAAAAACCATTAAGTGAAA | 1801 |
| | | | | TAACTATTAGATGTATAATT | |
| | | | | TGTCAAACGCCATTATGTCC | |
| | | | | TGAAGAAAA | |
| HPV52 | SA2 | 603 | 604 | ATAATATTATGGGTCGTTGG | 1802 |
| | | | | ACAGGGCGCTGTTCAGAGTG | |
| | | | | TTGGAGACCCCGACCTGTGA | |
| | | | | CCCAAGTGTAACGTCATGCG | |
| | | | | TGGAGACAAAGCAACTATAA | |
| | | | | AAGATTATA | |
| HPV52 | SA9 | 605 | 606 | ACGTTTTCCATATTTTTTTA | 1803 |
| | | | | CAGATGTCCGTGTGGCGGCC | |
| | | | | TAGTGAGGCCACTGTGTACC | |
| | | | | TGCCTCC | |
| HPV52 | SA10 | 607 | 608 | CGATTACTAACAGTAGGACA | 1804 |
| | | | | TCCCTATTTTTCTATTAAAA | |
| | | | | ACACCAGTAGTGGTAATGGT | |
| | | | | AAAAAAGTTTTAGTTCCCAA | |
| | | | | GG | |
| HPV52 | SA3 | 609 | 610 | CiACCGGCCAGATGGACAAG | 1805 |
| | | | | CAGAACAAGCCACAAGCAAT | |
| | | | | TACTACATTGTGACATATTG | |
| | | | | TCACAGTTGTGATAGCACAC | |
| | | | | TACGGCTATGCATTCATAGC | |
| | | | | ACTGCGACGG | |
| HPV52 | SD2 | 611 | 612 | GTGTGCCCCGGCTGTGCAC | 1806 |
| | | | | GGCTATAAACAACCCTGCAA | |
| | | | | TGGAGGACCCTGAAGGTACA | |
| | | | | GAGGGCGAAAGGGAGGGATG | |
| | | | | TACAGGCTGGTTTGAAGTAG | |
| | | | | AGGCAATAATAGAAA | |
| HPV56 | SD3 | 613 | 614 | CATTGGAAACTCTGGAAACA | 1807 |
| | | | | CCAGAACAGGTAGATGAAGA | |
| | | | | GGTACAGGGACGTGGGTGCG | |
| | | | | GGAATACACA | |
| HPV56 | SD1 | 615 | 616 | ATACCTTTAATTGATCTTAG | 1808 |
| | | | | ATTATCATGTGTATATTGCA | |
| | | | | AAAAAGAACTAACACGTGCT | |
| | | | | GAGGTATATAATTTTGCATG | |
| | | | | CACTGAATTAAAATTAGTGT | |
| | | | | ATAGGGATGATTTTCCT | |
| HPV56 | SA5 | 617 | 618 | ATTTCCATTAGATAATAATG | 1809 |
| | | | | GTAATCCTGTATATGAATTA | |
| | | | | AGTAATGTAAACTGGAAATG | |
| | | | | TTTCTTTACAAGGACGTGGT | |
| | | | | CCAGATTAAAT | |
| HPV56 | SD5 | 619 | 620 | ACTACGCCTGTAGTACATTT | 1810 |
| | | | | AAAAGGTGAACCTAACAGAT | |
| | | | | TAAAATGTTGTAGATATCGA | |
| | | | | TTTCAAAAATATAAAACATT | |
| | | | | GTTTGTGGATGTAACATCA | |
| HPV56 | SA1 | 621 | 622 | ATAACTAAAAAACAGTTATC | 1811 |
| | | | | iTGATTTATTAATAAGGTGC | |
| | | | | TACAGATGTCAAAGTCCG | |
| HPV56 | SA2 | 623 | 624 | ATCTAATAGCACATGGTTGG | 1812 |
| | | | | ACCGGGTCATGTTTGGGGTG | |
| | | | | CTGGAGACAAACATCTAGAG | |
| | | | | AACCTAGAGAATCTACAGTA | |
| | | | | TAATCATGCATGGTAAAG | |
| HPV56 | SA9 | 625 | 626 | CCTGTGTATTTTTTTAGACG | 1813 |
| | | | | TAGGCGCCGTAAACGTATTC | |
| | | | | CCTATTTTTTTGCAGATGGC | |
| | | | | GACGTGGCGGCCTAGTGAAA | |
| | | | | ATAAGGTGTATCTACC | |
| HPV56 | SA10 | 627 | 628 | TTGCTTGCCGTAGGACATCC | 1814 |
| | | | | CTATTACTCTGTGACTAAGG | |
| | | | | ACAATACCAAAACAAACATT | |
| | | | | CCCAAAGTTAGTGCATA | |
| HPV56 | SA3 | 629 | 630 | CATACGTGTTACCTAATACA | 1815 |
| | | | | CGTACCTTGTTGTGAGTGTA | |
| | | | | AGTTTGTGGTGCAGTTGGAC | |
| | | | | ATTCAGAGTACCAAA | |
| HPV56 | SD2 | 631 | 632 | GCGCATCAAGTAACTAACTG | 1816 |
| | | | | CAATGGCGTCACCTGAAGGT | |
| | | | | ACAGATGGGGAGGGGAAGGG | |
| | | | | ATGTTGTGG | |
| HPV58 | SD3 | 633 | 634 | ATGGCAATACTGAAGTGGAA | 1817 |
| | | | | ACTGAGCAGATGGCACACCA | |
| | | | | GGTAGAAAGCCAAAATGGCG | |
| | | | | ACGCAGAC | |
| HPV58 | SD1 | 635 | 636 | GTGCATGAAATCGAATTGAA | 1818 |
| | | | | ATGCGTTGAATGCAAAAAGA | |
| | | | | CTTTGCAGCGATCTGAGGTA | |
| | | | | TATGACTTTGTATTTGCAGA | |
| | | | | TTTAAGAATAGTGTATAGAG | |
| | | | | ATGGAAATCCA | |
| HPV58 | SA4 | 637 | 638 | AAAACATAGGGCATTAGTAC | 1819 |
| | | | | AATTAAAATGTCCACCATTA | |
| | | | | ATAATTACCTCAAATACAAA | |
| | | | | TGCAGGCAAAGATTCACGAT | |
| | | | | GGCCATATTTGCACAGTAGA | |
| | | | | CTAACAGTATT | |
| HPV58 | SA6 | 639 | 640 | GTCGGGTAATTGTATGTCCT | 1820 |
| | | | | ACATCTATACCTAGTGATCA | |
| | | | | AATATCCACTACTGAAACTG | |
| | | | | CTGACCCAAAGACCACCGAG | |
| | | | | GCC | |
| HPV58 | SD5 | 641 | 642 | GTACATACAAAGGGCGGAAC | 1821 |
| | | | | GTGTGTAGTTCTAAAGTTTC | |
| | | | | ACCTATCGTGCATTTAAAAG | |
| | | | | GTGACCCAAATAGTTTAAAA | |
| | | | | TGTTTAAGATATAGATTAAA | |
| | | | | ACCATTTAAAGACTTATAC | |
| HPV58 | SA1 | 643 | 644 | CTAAAAAAGTGTTTAAATGA | 1822 |
| | | | | AATATTAATTAGATGTATTA | |
| | | | | TTTGTCAAAGACCATTGTGT | |
| | | | | CCACAAGAAAAAAAAAGGCA | |
| | | | | TGTGGATTTAA | |
| HPV58 | SA9 | 645 | 646 | CTAAAAAAGTGTTTAAATGA | 1823 |
| | | | | AATATTAATTAGATGTATTA | |
| | | | | TTTGTCAAAGACCATTGTGT | |
| | | | | CCACAAGAAAAAAAAAGGCA | |
| | | | | TGTGGATTTAA | |
| HPV58 | SD2 | 647 | 648 | GTGTGCCCTAGCTGTGCACA | 1824 |
| | | | | GCAATAAACACCATCTGCAA | |
| | | | | TGGATGACCCTGAAGGTACA | |
| | | | | AACGGGGTAGGGGCGGGCTG | |
| | | | | TACTGGCTGGTTTGAGG | |
| HPV59 | SD3 | 649 | 650 | GCGGCTATGGCTATTCTGAA | 1825 |
| | | | | GTGGAAATGCTCGAGACTCA | |
| | | | | GGTAACCGTGGAGAATACTG | |
| | | | | GAAATGGGGATAGCAATGGC | |
| | | | | A | |
| HPV59 | SD1 | 651 | 652 | GGAACTGCAAGAAAGAGAGG | 1826 |
| | | | | TATTTGAATTTGCTTTTAAT | |
| | | | | GACTTATTTATAGTGTATAG | |
| | | | | AGACTGTACACC | |
| HPV59 | SA4 | 653 | 654 | ATTAAATGTCCACCAATGCT | 1827 |
| | | | | TATTACATCAAATACAAATC | |
| | | | | CAGTTACAGATAACAGGTGG | |
| | | | | CCATATTTAAATAGCAGATT | |
| | | | | AATGGTATTTAAATT | |
| HPV59 | SD5 | 655 | 656 | CAACCCGCGACGGCACATCC | 1828 |
| | | | | CTTGCAGTAACACTACGCCT | |
| | | | | ATAATACACTTAAAAGGTGA | |
| | | | | CAAAAATGGCCTTAAGTGTT | |
| | | | | TAAGGTATAGATTAAGAAAA | |
| | | | | GTACACTGGTTATT | |
| HPV59 | SA9 | 657 | 658 | CTATTTTTTTACAGATGGCT | 1829 |
| | | | | CTATGGCGTTCTAGTGACAA | |
| | | | | CAAGGTGTATCTACCTCCAC | |
| | | | | CTTCGGTAGCTAAG | |
| HPV59 | SA10 | 659 | 660 | CACGCAGGCAGTTCCAGACT | 1830 |
| | | | | TCTTACAGTTGGACATCCAT | |
| | | | | ATTTTAAAGTACCTAAAGGT | |
| | | | | GGTAATGGTAGACAGGATGT | |
| | | | | TCCTAAGGTGTCTGCATATC | |
| | | | | AATACAGAGTATTTAGGGTT | |
| HPV59 | SA3 | 661 | 662 | AGCTAGACGAGCTGAACCAC | 1831 |
| | | | | AGCGTCACAACATTGTGTGT | |
| | | | | GTGTGTTGTAAGTGTAATAA | |
| | | | | TCAACTTCAGCTAGTAGTAG | |
| | | | | AAACCTCGCAAG | |
| HPV59 | SD2 | 663 | 664 | GCAGCAAACCAGTAACCTGC | 1832 |
| | | | | AATGGCCGATTCGGAAGGTA | |
| | | | | CAGATGGGGAAGGGACGGGG | |
| | | | | TGCAATGGATGGTTTTTTGT | |
| | | | | GCAGGCAATAGTAGATAAA | |
| HPV66 | SD3 | 665 | 666 | CATTGGAAACATTGGAAACA | 1833 |
| | | | | TCACAACAGGTAGAATACGA | |
| | | | | AAAGGGAAATGGGTGCGGGA | |
| | | | | GCTCACAAAATGGAGGCTCG | |
| | | | | CAAAA | |
| HPV66 | SD1 | 667 | 668 | AATACCTTTACTTGATCTTA | 1834 |
| | | | | GATTATCATGTGTATACTGC | |
| | | | | AAAAAGGAACTTACAAGTTT | |
| | | | | AGAGCTATATAGGTTTGCAT | |
| | | | | GTATTGAGTTAAAACTAGTA | |
| | | | | TATAGAAACAATTGG | |
| HPV66 | SA6 | 669 | 670 | GGGGTGGATTACAGAGGCAT | 1835 |
| | | | | ATATTATATGCATGATGGCC | |
| | | | | ACAAAACATATTACACAGAC | |
| | | | | TTTGAACAGGAGGCCAAAAA | |
| | | | | ATATGGGTGTACAAACATAT | |
| | | | | GGGAAGTACAT | |
| HPV66 | SD5 | 671 | 672 | GGTGATAAAACTACGCCTGT | 1836 |
| | | | | AATCCATTTAAAAGGTGAAG | |
| | | | | CTAATAGATTAAAGTGTTGT | |
| | | | | AGATACAGATTTCAAAAATA | |
| | | | | TAAAACATTATTTACAGATG | |
| | | | | TA | |
| HPV66 | SA1 | 673 | 674 | GTTATCTGATTTATCAATAA | 1837 |
| | | | | GGTGCTACCGATGTCAATGT | |
| | | | | CCGTTAACACCGGAGGAAAA | |
| | | | | ACAA | |
| HPV66 | SA9 | 675 | 676 | TATTTTTTTAAACGTAGGCG | 1838 |
| | | | | CCGTAAACGTATTCCCTATT | |
| | | | | TTTTTGCAGATGGCGATGTG | |
| | | | | GCGGCCTAGTGACAATAAGG | |
| | | | | TGTACCTACC | |
| HPV66 | SA3 | 677 | 678 | CAACATAAGTGTTACCTAAT | 1839 |
| | | | | TCACGTACCTTGTTGTAAGT | |
| | | | | GTGAGTTGGTGGTGCAGTTG | |
| | | | | GACATTCAGAGTACCAA | |
| HPV66 | SD2 | 679 | 680 | GCGCATCATCTAAATAACTG | 1840 |
| | | | | CAATGGCATCACCTGAAGGT | |
| | | | | ACAGATGGGGAGGGGATGGG | |
| | | | | ATGTTGTGG | |
| HPV68 | SD3 | 681 | 682 | CCGGACAGCGGCTATGGCAA | 1841 |
| | | | | TATGGAAGTGGAAACTAACT | |
| | | | | CGGAGGTAACTGTAGCACCT | |
| | | | | AATATAAATGGGGAGGATGG | |
| | | | | GGAAAATGAAGGGGAA | |
| | | | | AATGGCGACAGT | |
| HPV68 | SD1 | 683 | 684 | GTTACAATAGACTGTGTCTA | 1842 |
| | | | | TTGCAGAAGGCAACTACAAC | |
| | | | | GGACAGAGGTATATGAATTT | |
| | | | | GCCTTTAGTGACCTATGTGT | |
| | | | | AGTGTATAGAGACGGGG | |
| HPV68 | SA4 | 685 | 686 | AATAACATCCAATACTAACC | 1843 |
| | | | | CTGTAGAAGACAATAGGTGG | |
| | | | | CCGTATTTACATAGTAGACT | |
| | | | | AACCGTGTTTAAATTTC | |
| HPV68 | SA1 | 687 | 688 | ACCATAACTAATACAAAGTT | 1844 |
| | | | | ATATAATTTATTGATAAGGT | |
| | | | | GCATGAGTTGCCTGAAACCA | |
| | | | | TTGTGTCCAGCAGAAAAA | |
| HPV68 | SA6 | 689 | 690 | ATGGCAACATAATCCATTGT | 1845 |
| | | | | CCTGACTCTATGTGCAGTAC | |
| | | | | CACTGACGGAAAAGTATCCA | |
| | | | | CTACTGAATCTGTTGCCGAC | |
| HPV68 | SD5 | 691 | 692 | AAGACGGAGCCTTTGTTGTG | 1846 |
| | | | | GTGACACTACACCTATAGTG | |
| | | | | CATTTAAAAGGTGACAAAAA | |
| | | | | TGGATTAAAATGTCTTA | |
| HPV68 | SA9 | 693 | 694 | TCCAATTATTATTTATTACC | 1847 |
| | | | | ATTGTTATTCTTTTTATTAA | |
| | | | | AAAAACGTAAACACCTTCCT | |
| | | | | TATTTTTTTACAGATGGCAT | |
| | | | | TGTGGCGAGCTAGCGACAAC | |
| | | | | ATGGTGTATTTGCCTCCCCC | |
| | | | | C | |
| HPV68 | SA3 | 695 | 696 | CGGGACGAACAACAGCGTCA | 1848 |
| | | | | CAGAATTCAGTGTCTGTGTT | |
| | | | | GTAAGTGTAACAAGGCACTG | |
| | | | | CAACTAGTAGTAGAAGCGTC | |
| | | | | GCGGGAC | |
| HPV68 | SD2 | 697 | 698 | ACCCAGTAATCTGCAATGGC | 1849 |
| | | | | CAATTGTGAAGGTACCGATG | |
| | | | | GGGACGGGACGGGGTGTAAC | |
| | | | | GGATGGTTTTTGTAGAAGC | |
| | | | | AAT | |
| HPV73 | SD3 | 699 | 700 | GGACAGTGGATATGGCAATA | 1850 |
| | | | | CTGAAGTGGAAACTTACGAG | |
| | | | | ACAGAGGTACCGGGACTTGG | |
| | | | | GGCAGGGGTAGGGTGTTTAC | |
| | | | | AAAATG | |
| HPV73 | SD1 | 701 | 702 | ATACATGATATAAACCTGGA | 1851 |
| | | | | CTGTGTGTTTTGCCAACGTG | |
| | | | | GACTGTACAGATCTGAGGTA | |
| | | | | TATGATTTTGCATTTAGTGA | |
| | | | | TTTGTGTATTGTATATAGAA | |
| | | | | AGGATAAACCATATG | |
| HPV73 | SA4 | 703 | 704 | ATCAAATACAAATCCTAAAG | 1852 |
| | | | | CAGATGATACTTGGAAATAT | |
| | | | | TTACATAGTAGAATTAAGGT | |
| | | | | GTTTACTTTTTTAAATCCAT | |
| | | | | T | |
| HPV73 | SA6 | 705 | 706 | ATGGGTGGTCAGGTAATATG | 1853 |
| | | | | TTGTGCTCCTGTATCTAGCG | |
| | | | | CCTGTGAAGTATCCATTCCT | |
| | | | | GAAATTGTTAACCCACTGCA | |
| | | | | C | |
| HPV73 | SD5 | 707 | 708 | CCTGTACCCAGTGTACTACA | 1854 |
| | | | | CATAATGTTGCGCCAATAGT | |
| | | | | GCATTTAAAAGGTGACAAAA | |
| | | | | ACAGCTTAAAATGTTTTAGA | |
| | | | | TATAGATTGCATAAAGGCTA | |
| | | | | TTCACATTTATTTAAAAA | |
| HPV73 | SA1 | 709 | 710 | AAATTTAACTAACAAACAGT | 1855 |
| | | | | TATGTAATATTTTAATAAGG | |
| | | | | TGCGGAAAATGCCAAAAACC | |
| | | | | ATTATGTCCACTGGAAAAGC | |
| | | | | AAAAGCATGTAGATGAAAA | |
| HPV73 | SA9 | 711 | 712 | TATTATTTGTTAAAGCGCAA | 1856 |
| | | | | ACGTAAACGTCTGTCATATT | |
| | | | | CTTTTACAGATGTGGCGACC | |
| | | | | TACTGATGCAAAGGTATACC | |
| | | | | TGCCCCC | |
| HPV73 | SA10 | 713 | 714 | TGGCTGTGGGACACCCATAT | 1857 |
| | | | | TTTCCTATCAAGGATTCTCA | |
| | | | | AAAACGTAAAACCATAGTTC | |
| | | | | CTAAAGTTTCAGGTTTG | |
| HPV73 | SA3 | 715 | 716 | CAGCCATCTAGACAGACAAG | 1858 |
| | | | | CTGAACGAGAGTGTTACAGA | |
| | | | | ATAGTTACTGACTGCACGAA | |
| | | | | GTGTCAGTGCACAGTATGCC | |
| | | | | TTGCCATTGAAAGCAACAAA | |
| | | | | GCTGATTTAAGAGTGA | |
| HPV73 | SD2 | 717 | 718 | GCCCCAACTGTTCCAGAAAC | 1859 |
| | | | | CTATAAAAGAAGATGGCTGA | |
| | | | | TTCAGGTAATTGGGAAGGGA | |
| | | | | GGTGTACGGGATGGTTTAAT | |
| | | | | GTAGAAGCCATTGTAG | |
| HPV82 | SD3 | 719 | 720 | CACAAGTGGAGACTGTGGAA | 1860 |
| | | | | GGACCCTTACAGGTAGATGG | |
| | | | | GCAAAATGACGGGTCACAAC | |
| | | | | ATAGTATGTGTAGTGGCGGG | |
| | | | | GGGAGC | |
| HPV82 | SD1 | 721 | 722 | ATTCAGGTATTGTGTGTATA | 1861 |
| | | | | TTGTAAAAAGGAGTTGTGTA | |
| | | | | GAGCAGATGTGTATAATGTA | |
| | | | | GCATTTACAGAACTTAGGAT | |
| | | | | TGTATATAGG | |
| HPV82 | SA4 | 723 | 724 | TTGTATGCCCACCATTGCTT | 1862 |
| | | | | ATTACCTCAAATATCAATCC | |
| | | | | AAAAGAAGATCCAAATTTAA | |
| | | | | TGTATTTACATAGTAGAGTG | |
| | | | | ACAGTATTTCAATTTTTAAA | |
| | | | | TGCATTTCCATTTGACCCCC | |
| | | | | AT | |
| HPV82 | SA6 | 725 | 726 | TATATGTGTGGCAATGTAAT | 1863 |
| | | | | AACATGTCCTGAATATGTAT | |
| | | | | CTAGTACCTACAGCACCCCG | |
| | | | | TCACCCTCTACTACAACTG | |
| HPV82 | SD5 | 727 | 728 | GGAACTGCAGGCCCAAACAC | 1864 |
| | | | | CGGAGGGCACCTCAGTGCAA | |
| | | | | CTAAAACTGCGTTTATAGTT | |
| | | | | CATTTAAAAGGTGCAACAAA | |
| | | | | TTGTTTAAAATGTTTAAGAT | |
| | | | | ACAGATTTGCAAAACATAGA | |
| | | | | AATTTGT | |
| HPV82 | SA1 | 729 | 730 | AGAGGCCATTACTAACAAAA | 1865 |
| | | | | GTTTATATGAATTATTAATA | |
| | | | | AGGTGTCATAGATGTCAGAG | |
| | | | | ACCACTTGGGCCTGAAGAAA | |
| | | | | AG | |
| HPV82 | SA9 | 731 | 732 | CATATTTGTTACGCAAACGC | 1866 |
| | | | | CGTAAACGTATACCCTATTT | |
| | | | | TTTTGCAGATGGCTTTGTGG | |
| | | | | CGTACTAATGACAGCAAAGT | |
| | | | | GTATTTACCACCTGCACC | |
| HPV82 | SA3 | 733 | 734 | CAGCCAGCCAGACAAGCTGG | 1867 |
| | | | | ACAGGATACGTGTTACAGAA | |
| | | | | TTAAAGTGCACTGTTGCAGG | |
| | | | | TGTTCGAGTGTTGTACAGCT | |
| | | | | CGCAGTGGAAAGCAGTG | |
| | | | | GAGACAGCC | |
| HPV82 | SD2 | 735 | 736 | CATCGGCAATGGACAGTGAA | 1868 |
| | | | | GGTACAGAGGATGAGGGGGC | |
| | | | | GGGGTGTACCGGGTGG | |

TABLE 2C

| | | | For- | | Re- |
|---|---|---|---|---|---|
| | | | ward | | verse |
| | | Forward | pri- | Reverse | pri- |
| | | primer | mer | primer | mer |
| | | nucleic | SEQ | nucleic | SEQ |
| HPV | Region | acid | ID | acid | ID |
| type | name | sequence | NO | sequence | NO |
| HPV16 | ctr11 | AACGTGTTGC | 737 | CATTCCCCAT | 738 |
| | | GATTGGTGTA | | GAACATGCTA | |
| | | TTG | | AACTTTG | |
| HPV16 | ctr12 | CGTGCTTTTT | 739 | GAGGCTGCTG | 740 |
| | | GCTTTGCTTT | | TTATCCACAA | |
| | | GT | | TAGTAAT | |
| HPV16 | ctr13 | CCTGTGTAGG | 741 | TCTATTATCC | 742 |
| | | TGTTGAGGTA | | ACACCTGCAT | |
| | | GGT | | TTGCT | |
| HPV16 | ctr14 | CCAGGCCCAT | 743 | AGGTCAGGAA | 744 |
| | | TTTGTAGCTT | | AACAGGGATT | |
| | | | | TGG | |
| HPV18 | ctr11 | TGGAGTAAAC | 745 | CATTTGTAAC | 746 |
| | | CCAACAATAG | | GCAACAGGGC | |
| | | CAGAAG | | TAAT | |
| HPV18 | ctr12 | CGTATGCATG | 747 | CATGTATATG | 748 |
| | | GGTATTGGTA | | CAATAGTAAC | |
| | | TTTGTG | | ATGGGCAA | |
| HPV18 | ctr13 | GAGGACGTTA | 749 | CCCTGTGATA | 750 |
| | | GGGACAATGT | | AAGGACGCGA | |
| | | GT | | TTT | |
| HPV18 | ctr!4 | CGCCCTAGTG | 751 | GGAGGATTGT | 752 |
| | | AGTAACAACT | | AGGATAAAAT | |
| | | GTATTT | | GGATGCT | |
| HPV31 | ctr11 | GTGAAACACC | 753 | TGCACATGCA | 754 |
| | | AGAATGGATA | | TTACTATCAC | |
| | | GAAAGAC | | TGTCA | |
| HPV31 | ctr12 | GCATTGTGCT | 755 | ACAACGTAAT | 756 |
| | | ATGCTTTTTG | | GGAGAGGTTG | |
| | | CTTTG | | CAATA | |
| HPV31 | ctr13 | GCTTAGTTTG | 757 | ACCACCGGCA | 758 |
| | | GGCCTGTGTT | | TATCTATTAG | |
| | | | | AGTTTTC | |
| HPV31 | ctr!4 | TGTGTGTGTT | 759 | CAACTTTTAC | 760 |
| | | GTGTATGTTG | | TATGGCGTGA | |
| | | TCCTT | | CACCTA | |
| HPV33 | ctr11 | CGGAGCCAAA | 761 | CGTTATCATA | 762 |
| | | CATGTGCATT | | TGCCCACTGT | |
| | | G | | ACCATT | |
| HPV33 | ctr12 | CCATTTCTAC | 763 | GTTGTGTCAT | 764 |
| | | CTATGCTTGG | | ATGCTGTGCA | |
| | | TTGCT | | TGAAA | |
| HPV33 | ctr13 | CATGTGTAGG | 765 | CCTATTATCA | 766 |
| | | CCTTGAAATA | | GCACCCGGTT | |
| | | GGTAGA | | GT | |
| | | G | | | |
| HPV33 | ctr14 | CTTGCCCTAC | 767 | CGGTTAGGCA | 768 |
| | | CCTGCATTG | | TACAAAATGG | |
| | | | | AGGAAAT | |
| HPV35 | ctr11 | GCTATGTATT | 769 | CATTCTGGTG | 770 |
| | | TCAGCTGCAA | | TTTCTCCATC | |
| | | GTATGCT | | AACCT | |
| HPV35 | ctr12 | CGTTCGCTAT | 771 | GCCAAATATT | 772 |
| | | TGCTATCTGT | | GTGCATGAGC | |
| | | GTCATTA | | GTTAATC | |
| HPV35 | Ctr13 | GGTACAGATA | 773 | GACATTCTCC | 774 |
| | | ACAGGGAATG | | TGCTTTTACC | |
| | | CATTTC | | TGGTTA | |
| | | T | | | |
| HPV35 | ctr14 | AACATTCCTA | 775 | TGGGTGGACC | 776 |
| | | CCTCAGCAGA | | ACAAGTATGA | |
| | | ACAC | | AAA | |
| HPV39 | ctr11 | AGGGTTACTG | 777 | CGTATCCCCT | 778 |
| | | TAGGAAAGGG | | GTTACCACAC | |
| | | ATTAAG | | TAATATTG | |
| | | T | | | |
| HPV39 | ctr12 | TTGGTGTGGT | 779 | CTCCAATGGT | 780 |
| | | TTGGTGTGTG | | GTGGTACGTA | |
| | | TATAT | | TAAGAA | |
| HPV39 | ctr13 | CCAGCCATTG | 781 | GCCTATAATG | 782 |
| | | GGTGTTGGTA | | CACAACTGTG | |
| | | | | TCTGTT | |
| HPV39 | ctr14 | CATTTTGTGG | 783 | CCTGGACAGG | 784 |
| | | CGACCGAAGT | | ATGATGAGTA | |
| | | | | ATAAGG | |
| HPV45 | ctr11 | GCAACGTTAT | 785 | GGTACGTGCA | 786 |
| | | ACGCCCATAT | | ACAATGTGCT | |
| | | CCAAT | | TAA | |
| HPV45 | ctr12 | TGCTTTTGCT | 787 | CATCACAGGT | 788 |
| | | TGGTTGTTGG | | ATGTTACACT | |
| | | T | | GTACTGT | |
| HPV45 | ctr13 | GGCATGTGTA | 789 | ACATCCTGCG | 790 |
| | | GGTATGGAAA | | TAATAACAGC | |
| | | TTGGT | | TGTAG | |
| HPV45 | ctr14 | ATTTCGGTTG | 791 | CAGTTGTGCA | 792 |
| | | CCTGTGGCTT | | AGCCATTGTT | |
| | | ATA | | TTAGT | |
| HPV51 | ctr11 | GATGGAGGCA | 793 | GTGTTTGGTG | 794 |
| | | ACTGGAGAGA | | GGCCATATAT | |
| | | AATT | | GACTAT | |
| HPV51 | ctr12 | AAGCCAATAT | 795 | AACACGTATT | 796 |
| | | GTGCTGCTAA | | GGGACAGCAG | |
| | | TTGTA | | TAG | |
| HPV51 | ctr13 | ACACCCCTCC | 797 | TGTACGCCAA | 798 |
| | | ACAGGCTAA | | CCTGCAACAA | |
| HPV51 | ctr14 | GGGTATTACA | 799 | GCTGCAGCTG | 800 |
| | | TTATCCCCGT | | TAACAAAATG | |
| | | AGGTCA | | GAA | |
| | | A | | | |
| HPV52 | ctr11 | CACCATCAGT | 801 | CTGTGACATT | 802 |
| | | TGCAGAAGGA | | AGTTTGGACA | |
| | | TTAAAA | | CTGTT | |
| | | G | | | |
| HPV52 | ctr12 | CAACACAAGC | 803 | CCTGCGCATA | 804 |
| | | CAATATTGCT | | CACCGATATA | |
| | | GCTA | | GAT | |
| HPV52 | ctr13 | GGACTATATG | 805 | GATGCAGGGC | 806 |
| | | TTTTGGGAGG | | GTTTTAGTTT | |
| | | TGGATT | | GG | |
| | | T | | | |
| HPV52 | ctr14 | TCGGTTGGTC | 807 | TTTAGGCGGG | 808 |
| | | TTGGCACAA | | ACAACAAGTG | |
| | | | | T | |
| HPV56 | ctr11 | CAGATGATAG | 809 | GCTGTTGTGC | 810 |
| | | CCAAATTGCG | | CCTTTTATAA | |
| | | TTTCA | | TGTCTAC | |
| HPV56 | ctr12 | TGCTACGCAT | 811 | GGATGTGGCT | 812 |
| | | ATATATTGCA | | ATAACAAACC | |
| | | ACCATT | | AAAACAAT | |
| | | GA | | | |
| HPV56 | Ctr13 | TGTACTCCCG | 813 | GTGTCTATCA | 814 |
| | | CTATGGGTGA | | TGTCCCCATC | |
| | | A | | CTCTA | |
| HPV56 | ctr14 | AATTCGGTTG | 815 | GGGTGCGGTA | 816 |
| | | CATGGCCTAG | | CTGTACATAA | |
| | | T | | TTCAAG | |
| HPV58 | ctr11 | CAATGGGACA | 817 | GGGCCACACA | 818 |
| | | ATGGATACAA | | GTAACATACA | |
| | | AGTAG | | ACT | |
| | | GT | | | |
| HPV58 | ctr12 | TCTATATATG | 819 | CATGTGCAGA | 820 |
| | | CTTGGTTGCT | | ACCAGTATAC | |
| | | GGTGTTG | | AGTTAGT | |
| HPV58 | ctr13 | CGTTTGGTCT | 821 | GCTGTGCGGG | 822 |
| | | GGGCATGTGT | | ATATCTGTTA | |
| | | A | | CTG | |
| HPV58 | ctr14 | TCTATGAGTA | 823 | GGAGGTAAAG | 824 |
| | | AGGTGCTGTC | | TAAAATGGAG | |
| | | CCTAAA | | GCAGTA | |
| | | T | | | |
| HPV59 | ctr11 | GTGCATGTTA | 825 | TCAAACACGC | 826 |
| | | ATTGAACCAC | | TATCATCAAC | |
| | | CCAAA | | TCCAT | |
| HPV59 | ctr12 | GTTGCAATGT | 827 | CATGGGCATA | 828 |
| | | CCCGCTTCTG | | TAGTAGTAAC | |
| | | | | AGTGGAA | |
| HPV59 | ctr13 | GCTGTGTACC | 829 | CTGTGTCTAC | 830 |
| | | TGCCATTGGA | | CATATCACCA | |
| | | | | TCTTCA | |
| HPV59 | ctr14 | GGTTGCACCC | 831 | GCAAAACTGG | 832 |
| | | AATGAGTAAG | | ACATTCAGGA | |
| | | GTA | | CAAAA | |
| HPV66 | ctr11 | AGACATAGAT | 833 | ATCACCCCCT | 834 |
| | | AGCAATGCAC | | TCATCTACTT | |
| | | AAGCA | | TACTACA | |
| HPV66 | ctr12 | GTTTGTCTGT | 835 | GCATGGCAAT | 836 |
| | | GTGTGTGCCA | | ATATACACAG | |
| | | TT | | TGTAGGT | |
| HPV66 | Ctr13 | GTAGGCCGAG | 837 | GTGCACATCC | 838 |
| | | GTCAACCTTT | | CACAATACAT | |
| | | A | | AACTG | |
| HPV66 | ctr14 | GGTTAGGTGG | 839 | CAAAAGGCTA | 840 |
| | | TGTTCCTTAC | | GGCAACCGAA | |
| | | TGTTTA | | TT | |
| HPV68 | ctr11 | CGACACGCCG | 841 | CGCTGCAGCA | 842 |
| | | GAATGGATAA | | TTACTATTAC | |
| | | | | AATCTG | |
| HPV68 | ctr12 | GGTGTGGTTT | 843 | GGTATACAGC | 844 |
| | | TGTGTATGCA | | AAACACCTCA | |
| | | TGT | | AATGGT | |
| HPV68 | Ctr13 | GCCTGTGTTG | 845 | TGCAACATTG | 846 |
| | | GTGTTGAAAT | | TCCCTACTGT | |
| | | AGGTA | | CTTTAG | |
| HPV68 | ctr14 | CCCTGTGACT | 847 | CCACACGGTA | 848 |
| | | AACATATGTC | | TAGTTTGCAA | |
| | | CTTGT | | CCAT | |
| HPV73 | ctr11 | GAACGCATGT | 849 | GCTGCACTAA | 850 |
| | | TAATTGAACC | | CGTTTGTCTT | |
| | | TCCAA | | TTAATCC | |
| HPV73 | ctr12 | TCGCTTGCAG | 851 | CATGGTAATG | 852 |
| | | TGTCTGTGTA | | TACAAGTGCC | |
| | | TATTT | | ATAGGA | |
| HPV73 | Ctr13 | TGTATTTTAG | 853 | CTCCAAAGCC | 854 |
| | | GTTGTAGGCC | | AACATCTATC | |
| | | TCCCTTA | | ATATCAC | |
| HPV73 | ctr14 | GTCGCCATTT | 855 | AGGAAACAAA | 856 |
| | | TACATGCATT | | CCCTGCCAAG | |
| | | AAGGT | | TT | |
| HPV82 | Ctr11 | CGTAGTACAG | 857 | CCCATTGTAC | 858 |
| | | CCGTTGCATT | | CATTTGCGAT | |
| | | G | | AGTT | |
| HPV82 | ctr12 | GCTGCTAAGT | 859 | CTGCTGCAAA | 860 |
| | | GTATATAGTT | | CACATATTGG | |
| | | ACTCGC | | GATT | |
| | | A | | | |
| HPV82 | Ctr13 | GGATGTGTTG | 861 | TCCTGTTGGT | 862 |
| | | GTGTTGAAGT | | CGTTGCCATT | |
| | | AGGTA | | | |
| HPV82 | ctr14 | CCTGTAGGTT | 863 | AAATCGGTCG | 864 |
| | | AAGGGTGGTG | | CCACAAAATG | |
| | | TT | | G | |

TABLE 2Cbis

| | | For- | | | |
|---|---|---|---|---|---|
| | | ward | | | |
| | | pri- | | | Ampli- |
| | | mer | Reverse | Amplicon | con |
| | | SEQ | primer | nucleic | SEQ |
| HPV | Region | ID | SEQ ID | acid | ID |
| type | name | NO | NO | sequence | NO |
| HPV16 | ctr11 | 737 | 738 | CTGCATTTGGACTTA | 1869 |
| | | | | CACCCAGTATAGCTG | |
| | | | | ACAGTATAAAAACAC | |
| | | | | TATTACAACAATATT | |
| | | | | GTTTATATTTACACA | |
| | | | | TT | |
| HPV16 | ctr12 | 739 | 740 | GTGCTTTTGTGTGTC | 1870 |
| | | | | TGCCTATTAATACGT | |
| | | | | CCGCTGCTTTTGTCT | |
| | | | | GTGTCTACATACACA | |
| | | | | TCATTAATAATATTG | |
| | | | | GT | |
| HPV16 | ctr13 | 741 | 742 | CGTGGTCAGCCATTA | 1871 |
| | | | | GGTGTGGGCATTAGT | |
| | | | | GGCCATCCTTTATTA | |
| | | | | AATAAATTGGATGAC | |
| | | | | ACAGAAAATGCTAGT | |
| | | | | GCTTATGC | |
| HPV16 | ctr14 | 743 | 744 | CAACCGAATTCGGTT | 1872 |
| | | | | GCATGCTTTTTGGCA | |
| | | | | CAAAATGTGTTTTTT | |
| | | | | TAAATAGTTCTATGT | |
| | | | | CAGCAACTATGGTTT | |
| | | | | AAACTTGTACGTTTC | |
| | | | | CTGCTTGCCATGCGT | |
| | | | | G | |
| HPV18 | ctr11 | 745 | 746 | GATTTAAAACACTAA | 1873 |
| | | | | TACAGCCATTTATAT | |
| | | | | TATATGCCCATATTC | |
| | | | | AATGTCTAGACTGTA | |
| | | | | AATGGGGAGTATTAA | |
| | | | | T | |
| HPV18 | ctr12 | 747 | 748 | TATATTGTGGTAATA | 1874 |
| | | | | ACGTCCCCTGCCACA | |
| | | | | GCATTCACAGTATAT | |
| | | | | GTATTTTGTTTTTTA | |
| HPV18 | ctr13 | 749 | 750 | CTGTAGATTATAAGC | 1875 |
| | | | | AGACACAGTTATGTA | |
| | | | | TTTTGGGCTGTGCCC | |
| | | | | CTGCTATTGGGGAAC | |
| | | | | ACTGGGCTAAAGGCA | |
| | | | | CTGCTTGT | |
| HPV18 | ctr14 | 751 | 752 | GTGTTTGTGGTATGG | 1876 |
| | | | | GTGTTGCTTGTTGGG | |
| | | | | CTATATATTGTCCTG | |
| | | | | TATTTCAAGTTATAA | |
| | | | | AACTGCACACCTTAC | |
| HPV31 | ctr11 | 753 | 754 | AAACAGTATTACAGC | 1877 |
| | | | | ATAGTTTTAATGACA | |
| | | | | CAACATTTGATTTGT | |
| | | | | CCCAAATGGTACAAT | |
| | | | | GGGCATATGACAATG | |
| | | | | ATGTTATGGATGATA | |
| | | | | GTGAAATTGCCTATA | |
| | | | | AATATGCACAATTAG | |
| | | | | C | |
| HPV31 | ctr12 | 755 | 756 | CTTTTGTGTGCTACT | 1878 |
| | | | | ATTTGTGTGTCTTGT | |
| | | | | CATACGTCCACTTGT | |
| | | | | GCTGTCTGTGTCGGT | |
| | | | | ATATGCAACACTACT | |
| | | | | ATTATTAATTGTGAT | |
| | | | | TTTATGGGT | |
| HPV31 | ctr13 | 757 | 758 | GGTTTAGAGGTAGGT | 1879 |
| | | | | CGCGGGCAGCCATTA | |
| | | | | GGTGTAGGTATTAGT | |
| | | | | GGTCATCCATTATTA | |
| | | | | AATAAATTTGATGAC | |
| | | | | ACT | |
| HPV31 | ctr14 | 759 | 760 | ATATACACCCTATTA | 1880 |
| | | | | GTAACATACTATTAC | |
| | | | | TATTTTATAAACTAT | |
| | | | | TGTTCCTACTTGTTC | |
| | | | | CTACTTGTTCCTGCT | |
| | | | | CCTCCCAATAGTCAT | |
| | | | | GTACTTATTTCTGCC | |
| | | | | TATAATT | |
| HPV33 | ctr11 | 761 | 762 | ATATACACCCTATTA | 1881 |
| | | | | GTAACATACTATTAC | |
| | | | | TATTTTATAAACTAT | |
| | | | | TGTTCCTACTTGTTC | |
| | | | | CTACTTGTTCCTGCT | |
| | | | | CCTCCCAATAGTCAT | |
| | | | | GTACTTATTTCTGCC | |
| | | | | TATAATT | |
| HPV33 | ctr12 | 763 | 764 | GGTGTTGGTATTGCTG | 1882 |
| | | | | CTTTGGGTGTTTGTG | |
| | | | | GGATCTCCTTTAAAA | |
| | | | | ATTTTTTTTTGCTAT | |
| | | | | TTGTTGTTTTTATAT | |
| | | | | TTACCAATGATGTGT | |
| | | | | ATTAAT | |
| HPV33 | Ctr13 | 765 | 766 | GGCAGCCATTAGGCG | 1883 |
| | | | | TTGGCATAAGTGGTC | |
| | | | | ATCCTTTATTAAACA | |
| | | | | AATTTGATGACACTG | |
| | | | | AAACCGGTAACAAGT | |
| | | | | ATCCTGG | |
| HPV33 | ctr14 | 767 | 768 | CAATGTACCTACCTT | 1884 |
| | | | | TATTTCCCTATATTT | |
| | | | | GTAGTACCTACATGT | |
| | | | | TTAGTATTGCTTTAC | |
| | | | | CTTTTGACATACTAG | |
| | | | | TGTCCATATTGTACA | |
| HPV35 | ctr11 | 769 | 770 | AATACAACCACCAAA | 1885 |
| | | | | ATTACGTAGTACCCC | |
| | | | | AGCTGCGTTATATTG | |
| | | | | GTTTAAAACAGCAAT | |
| | | | | GTCAAATATTAGTG | |
| HPV35 | ctr12 | 771 | 772 | TACTCAGCATTAATA | 1886 |
| | | | | TTACTGGTTTTAATA | |
| | | | | CTGTGGGTTACTGTA | |
| | | | | GCAACACCACTACGT | |
| | | | | TGCTTTTGTTGTTTT | |
| | | | | CTTTGCTTTTTGTAT | |
| | | | | ATACCTATGGGAAT | |
| HPV35 | Ctr13 | 773 | 774 | ATGGATTATAAACAA | 1887 |
| | | | | ACACAATTGTGTTTA | |
| | | | | ATAGGTTGTAGGCCT | |
| | | | | CCTATAGGTGAACAT | |
| | | | | TGGGGAAAAGGCACA | |
| | | | | CCTTGTAATGC | |
| HPV35 | ctr14 | 775 | 776 | TTAATCCTTGTGTTC | 1888 |
| | | | | CTGATATATATTGTT | |
| | | | | TGCCAACTTTATATT | |
| | | | | GGC | |
| | | | | TTTTGCCAATCTTTA | |
| | | | | AACTTGATTCATCTT | |
| | | | | GCAGTATTAGTCAT | |
| HPV39 | ctr11 | 777 | 778 | ACATTGTTACATGTT | 1889 |
| | | | | CCAGAAAGTTGTATG | |
| | | | | CTTCTGGAGCCTCCT | |
| | | | | AAACTGCGCAGCCCT | |
| | | | | GTAGCAGCACTATAT | |
| | | | | TGGTATCGCACAGGT | |
| | | | | ATATC | |
| HPV39 | ctr12 | 779 | 780 | ATATATGTTGCAATG | 1890 |
| | | | | TCCCGCTTTTGCCGT | |
| | | | | CTGTGCATGTGTGTG | |
| | | | | CGTATGTGTGGATAA | |
| | | | | TTGTGTTTGTGTTTA | |
| HPV39 | ctr13 | 781 | 782 | TTAGTGGACACCCAT | 1891 |
| | | | | TATATAATAGACAGG | |
| | | | | ATGATACTGAAAACT | |
| | | | | CACCATTTTCATCAA | |
| | | | | CCACCAATAAGGACA | |
| | | | | GTAGGGATAATGTGT | |
| | | | | CTGTGGATTATA | |
| HPV39 | ctr14 | 783 | 784 | TCAGCAAAAACATGT | 1892 |
| | | | | CTTTACCTTAGGTTC | |
| | | | | ACCCTGCATAGTTGG | |
| | | | | CACTGGTAACAGTTT | |
| | | | | TACTGGCGCG | |
| HPV45 | ctr11 | 785 | 786 | GTTTAGATTGTAAAT | 1893 |
| | | | | GGGGAGTATTAATAT | |
| | | | | TAGCTTTATTAAGAT | |
| | | | | ATAAATGTGGCAAAA | |
| | | | | ATAGACTAACTGTTG | |
| | | | | CAAAAGGC | |
| HPV45 | ctr12 | 787 | 788 | GTTTCTTTTTATAGT | 1894 |
| | | | | TGTTATTACATCCCC | |
| | | | | ATTAACAGCATTTGC | |
| | | | | TGTATACATTTGTTG | |
| | | | | CTATTTACTACCTAT | |
| | | | | GTTTGTATTACATAT | |
| | | | | GCATGCTTTACACAC | |
| | | | | CATACAATAATTACT | |
| | | | | ATAATGT | |
| HPV45 | ctr13 | 789 | 790 | CGTGGGCAGCCTTTA | 1895 |
| | | | | GGTATTGGCCTAAGT | |
| | | | | GGCCATCCATTTTAT | |
| | | | | AATAAATTGGATGAT | |
| | | | | ACAGAAAGTGCTCAT | |
| | | | | GCAG | |
| HPV45 | ctr14 | 791 | 792 | TGTGACCTTTTAAAC | 1896 |
| | | | | ATAATACCTAAACTG | |
| | | | | GCACATTTACAACCC | |
| | | | | CTACATAGTTTAACC | |
| | | | | TACTGGCGCGCCTTC | |
| | | | | TTGGCGTACATGTGG | |
| | | | | CACACCTGGTATTAG | |
| | | | | TCATTTTCCTGTCCA | |
| | | | | GGTGT | |
| HPV51 | ctr11 | 793 | 794 | GCTAAATTTTTAAGA | 1897 |
| | | | | TATCAAGGTGTAAAC | |
| | | | | TTTATGTCCTTTATT | |
| | | | | CAAATGTTTAAACAG | |
| | | | | TTTTTAAAAGGAACA | |
| | | | | CCAAAACACAATTGC | |
| HPV51 | ctr12 | 795 | 796 | TAGACATATTGTAAC | 1898 |
| | | | | CATTGCAGTGTTTAT | |
| | | | | TATTTTGCTATTTGT | |
| | | | | GCTTTGCTTGTGTGT | |
| | | | | GTGTCTTGTGTTGTG | |
| | | | | TTGTTTGTTGCCG | |
| HPV51 | Ctr13 | 797 | 798 | GCCAGATCCTTTGGC | 1899 |
| | | | | CAAATATAAATTTTG | |
| | | | | GGATGTTGATTTAAA | |
| | | | | GGAACGATTTTCTTT | |
| | | | | AGATTTAGACCAATT | |
| | | | | TGCATTGGGTCGCAA | |
| | | | | GTTT | |
| HPV51 | ctr14 | 799 | 800 | GGGTGGTGTTTCGGT | 1900 |
| | | | | GGCGTCCCTATTGCC | |
| | | | | CTACCCATTTTTTGC | |
| | | | | AG | |
| | | | | CACAACAGTTTATAT | |
| | | | | TTGTGCTATTTAGTT | |
| | | | | ATACTTTGTAGC | |
| HPV52 | ctr11 | 801 | 802 | TATTAATACAGCCCT | 1901 |
| | | | | ATAGCATATATGCCC | |
| | | | | ATTTGCAATGTTTAA | |
| | | | | CATGTGACAGAGGCG | |
| | | | | TGCTTATACTGCTGC | |
| | | | | TAATTAGGTTTAAAT | |
| | | | | GTGGAAAAAACAGAT | |
| | | | | T | |
| HPV52 | ctr12 | 803 | 804 | TTGTGTATATATAAC | 1902 |
| | | | | AATGTTAGGATTATT | |
| | | | | TGTATTTTGTTTTAT | |
| | | | | TTTGCTTATGGTGTT | |
| | | | | TTGTGCAGTGCTTAG | |
| | | | | GCCGCTCTTGCT | |
| HPV52 | Ctr13 | 805 | 806 | AAAAGAAAAGTTTTC | 1903 |
| | | | | TGCAGATTTAGATCA | |
| | | | | GTTTCCTTTAGGTAG | |
| | | | | GAAGTTTTTGTTACA | |
| | | | | GGCAGGGCTACAGGC | |
| | | | | TAGGC | |
| HPV52 | ctr14 | 807 | 808 | CTTTGGTTGTCCTTG | 1904 |
| | | | | GCACAGTAACAACTA | |
| | | | | TTTTTATATAAGTTT | |
| | | | | CAGCAAACTGCTTAA | |
| | | | | TCCTTTGGTTTCCTG | |
| | | | | CAGTCCACTGGTCT | |
| HPV56 | ctr11 | 809 | 810 | ATATGCACAATTAGC | 1905 |
| | | | | AGATGTAGACAGCAA | |
| | | | | TGCACAAGCCTTTTT | |
| | | | | AAAAAGCAATATGCA | |
| | | | | GGCAAAATATGTAAA | |
| | | | | GGATTGTGGAATAAT | |
| | | | | GT | |
| HPV56 | ctr12 | 811 | 812 | TTTTTGTGTTATTGG | 1906 |
| | | | | TGTGTTTGCGCTTTG | |
| | | | | CTTTTGTGTTTGTTT | |
| | | | | GCTTGTGTGTCATGT | |
| | | | | TGTCCCGCTTTTGCT | |
| | | | | ATCTGCCTCTGTGTT | |
| | | | | TTCCAGTTGTATATT | |
| | | | | ATTAATAAT | |
| HPV56 | ctr13 | 813 | 814 | CATTGGACTAAAGGT | 1907 |
| | | | | GCTGTGTGTAAGTCC | |
| | | | | ACACAAGTTACCACA | |
| | | | | GGGGACTGCCCGCCT | |
| | | | | CTTGCATTAATTAAT | |
| | | | | ACACCTA | |
| HPV56 | ctr14 | 815 | 816 | GCCATTATTTAAACT | 1908 |
| | | | | AAAAGGAATTCGGTT | |
| | | | | GCATGGCCTAGTGCC | |
| | | | | ATTATTTAAACCAAA | |
| | | | | AGGCCCTTTTCAGCA | |
| | | | | GAACAGTTAATCCTT | |
| | | | | TGGCATATTGCCGTT | |
| | | | | TCCTGTGTTTTATA | |
| HPV58 | ctr11 | 817 | 818 | GTGAAAAAACAAATG | 1909 |
| | | | | ATGGAGGTAATTGGA | |
| | | | | GACCAATAGTACAAT | |
| | | | | TTTTAAGATATCAAA | |
| | | | | ATATTGAATTTACAG | |
| | | | | CATTTTTAGTTGCAT | |
| | | | | TTAAACAGTTTTTAC | |
| | | | | AAGGTGTACCAAAAA | |
| | | | | AA | |
| HPV58 | ctr12 | 819 | 820 | GTGTTGCTGCTTTGG | 1910 |
| | | | | GTGTCTGTGGGGTCG | |
| | | | | GCTCTACGAATTTTT | |
| | | | | TTCTGTTACTTAATA | |
| | | | | TTTTTATATATACCA | |
| | | | | ATGATGTGTATTAAT | |
| | | | | TTTCATGCACAATAC | |
| | | | | TTAACCCAACAAG | |
| HPV58 | Ctr13 | 821 | 822 | GGCCTTGAAATAGGT | 1911 |
| | | | | AGGGGACAGCCATTG | |
| | | | | GGTGTTGGCGTAAGT | |
| | | | | GGTCATCCTTATTTA | |
| | | | | AATAAATTTGATGAC | |
| | | | | ACTGAAAC | |
| HPV58 | ctr14 | 823 | 824 | TGCCCTACCCTGCCC | 1912 |
| | | | | TGCCTATTATGCATA | |
| | | | | CCTATGTAATAGTAT | |
| | | | | TTGTATGATATGTAT | |
| | | | | TTTATAGTTTTTAAC | |
| | | | | AG | |
| HPV59 | ctr11 | 825 | 826 | TTGCGTAGTGGTGTT | 1913 |
| | | | | GCAGCACTATATTGG | |
| | | | | TACAGAACAGGAATG | |
| | | | | TCCAATATTAGTGAA | |
| | | | | GTTATAGGGGAAACG | |
| | | | | CCCGAATGGATACAA | |
| | | | | AGACTAACAATTATA | |
| | | | | CAAC | |
| HPV59 | ctr12 | 827 | 828 | CAATCTGTCTATATG | 1914 |
| | | | | TGTGCATATACATGG | |
| | | | | TTACTAGTATTTGTG | |
| | | | | TATATTGTGGTTATC | |
| | | | | ACCTCCTCATATGAG | |
| | | | | TGTTTTTTACTATAT | |
| | | | | ATATTGTTTTTTATA | |
| | | | | A | |
| HPV59 | ctrB | 829 | 830 | GAACACTGGACAAAG | 1915 |
| | | | | GGCACTGCTTGTAAG | |
| | | | | CCTACTACTGTGGTT | |
| | | | | CAGGGCGATTGTCCT | |
| | | | | CCACTAGAATTAATA | |
| | | | | AATACACCAAT | |
| HPV59 | ctr14 | 831 | 832 | CTGTCCCTTTATTGT | 1916 |
| | | | | TTCTTTGTCCTTATT | |
| | | | | ACACATTATTACACA | |
| | | | | TTGCCCTACTTACAT | |
| | | | | AGGTGTGTTTGTTCC | |
| | | | | TTCA | |
| HPV66 | ctr11 | 833 | 834 | TTTTTAAAAAGTAATA | 1917 |
| | | | | TGCAAGCAAAATATG | |
| | | | | TAAAGGATTGTGGAA | |
| | | | | TAATGTGTAGACATT | |
| | | | | ACAAAAGGGCACAGC | |
| | | | | AACAGCAAATGAATA | |
| | | | | TGTGCCAGTGGATAA | |
| | | | | AGCATATA | |
| HPV66 | ctr12 | 835 | 836 | TTGTCCCGCTTTTGC | 1918 |
| | | | | TATCTGCATCTTTAT | |
| | | | | TTACAAGTTGTCTTA | |
| | | | | TACTAATTATTTTAT | |
| | | | | TTTGGTTTGTTGTGG | |
| | | | | CTACATCATTTTTTG | |
| | | | | ATACTTTTATACTGT | |
| | | | | TTTTACTATTTTTTT | |
| | | | | ATAT | |
| HPV66 | Ctr13 | 837 | 838 | GGTGCTGGGTTAAGT | 1919 |
| | | | | GGTCATCCATTATTT | |
| | | | | AATAGGCTGGATGAC | |
| | | | | ACTGAGGTCTCTAAT | |
| | | | | TTAGCAGGTAATAAT | |
| | | | | GTTATAGAAGATAGC | |
| | | | | CGGGACAATATATCT | |
| | | | | GTTGATTGTAAACAA | |
| | | | | ACC | |
| HPV66 | ctr14 | 839 | 840 | ATGTTATATTAAATA | 1920 |
| | | | | GGTTGTTTGTATGCA | |
| | | | | CTATAGTAACACACC | |
| | | | | AAACTCCATTTTAGT | |
| | | | | GCTGTACGCCATTTT | |
| | | | | ATGCATGCAACCG | |
| HPV68 | ctr11 | 841 | 842 | AAAGATTAACCATAA | 1921 |
| | | | | TACAACATGGAATAG | |
| | | | | ATGATAGTGTATTTG | |
| | | | | ATTTATCAGACATGG | |
| | | | | TACAATGGGCATTTG | |
| | | | | ATAATGAGTTAACAG | |
| | | | | ATGACAGTGATATAG | |
| | | | | CATTTCAATATGCTA | |
| | | | | TGTTAG | |
| HPV68 | ctr12 | 843 | 844 | ATATATGTTGCACTG | 1922 |
| | | | | TCCCGCTTTTGCAGT | |
| | | | | CTGTGCATGTGTGTG | |
| | | | | TGTATGTGTGGATAT | |
| | | | | TTGTGTTTGTGTTTA | |
| | | | | TATTAGTTAGAACTA | |
| | | | | C | |
| HPV68 | Ctr13 | 845 | 846 | GGGGGCAGCCATTGG | 1923 |
| | | | | GCGTTGGCCTTAGTG | |
| | | | | GGCATCCACTATATA | |
| | | | | ATAGGCTGGATGATA | |
| | | | | CTGAAAATTCCCCGT | |
| | | | | TTTCCTCTAATAAAA | |
| | | | | CTC | |
| HPV68 | ctr14 | 847 | 848 | TTTACATATAATAGG | 1924 |
| | | | | ACTGCAACATTTCAT | |
| | | | | ACATAATTTGTAGCC | |
| | | | | CTACCCTAAGGTGTG | |
| | | | | TTACATTATATGCAA | |
| | | | | TATATTT | |
| HPV73 | ctr11 | 849 | 850 | GACTACGAAGTACAC | 1925 |
| | | | | CATGTGCATTATATT | |
| | | | | GGTATAGAACTAGTT | |
| | | | | TATCAAATATTAGTG | |
| | | | | AAATAGTAGGAGACA | |
| | | | | CACCTGAGT | |
| HPV73 | ctr12 | 851 | 852 | ACCCATGGTTATTGG | 1926 |
| | | | | TATTGATTATAATAA | |
| | | | | CCTTTATACATGTAT | |
| | | | | CA | |
| HPV73 | Ctr13 | 853 | 854 | GGGGAACATTGGGGT | 1927 |
| | | | | CCAGGCACGCCATGT | |
| | | | | ACTTCACAAACTGTT | |
| | | | | AATACTGGTGATTGT | |
| | | | | CCCCCACTGGAATTA | |
| | | | | AAGAACACCCCTATA | |
| | | | | CAGGATG | |
| HPV73 | ctr14 | 855 | 856 | AAAAAGGGCAACCGA | 1928 |
| | | | | TTTCGGTTGCACAGT | |
| | | | | AAAACATGTTTTAAT | |
| | | | | GTGTTTTGCTGTTGT | |
| | | | | AGCAAAATAGTTGTA | |
| | | | | CTGTTTTTGGCTTCC | |
| | | | | TGCAGGC | |
| HPV82 | ctr11 | 857 | 858 | TACTTTTATAGAACA | 1929 |
| | | | | GGAATATCAAACATT | |
| | | | | AGTAGCACATATGGC | |
| | | | | GAAACACCAGAATGG | |
| | | | | ATTACAAGACAAACA | |
| | | | | CAACTACAGCACAGT | |
| | | | | TTTGATGATAGCACG | |
| | | | | TTTG | |
| HPV82 | ctr12 | 859 | 860 | ACCATTGCGGTGTTT | 1930 |
| | | | | TTGGTGTGTTTATTT | |
| | | | | GTGCTTTGCGTGTGT | |
| | | | | GTGTGTCTTGTGTTG | |
| | | | | TGTTGTTTGTTGCCG | |
| | | | | CTATTGC | |
| HPV82 | ctr13 | 861 | 862 | GGGGTCAGCCGTTAG | 1931 |
| | | | | GTGTTGGCCTTAGTG | |
| | | | | GTCATCCTTTATTTA | |
| | | | | ATAAGTATGATGATA | |
| | | | | CTGAAAACTCTAGGT | |
| | | | | TTGCC | |
| HPV82 | ctr14 | 863 | 864 | TAGGTGGCGTCCCTA | 1932 |
| | | | | TTGCCCTACCCATAT | |
| | | | | TTGTGGCTTGCAGCA | |
| | | | | CACTTGTATATATAT | |
| | | | | GTTCTTGCTGTATTG | |
| | | | | CATGTACCACAGGAT | |
| | | | | TCCATTTTGTTTTTT | |
| | | | | CCTGCAG | |

TABLE 2D

| | | | Forward | | Reverse |
|---|---|---|---|---|---|
| | | Forward primer | primer | Reverse primer | primer |
| HPV | | nucleic acid | SEQ ID | nucleic acid | SEQ ID |
| type | Region name | sequence | NO | sequence | NO |
| HPV16 | bkpt1- | AGTACAGACCTACGTGACCATATAGA | 865 | CTGAGAAGCCCTGCCCTTC | 866 |
| | MYC_001_exon1 | C | | | |
| HPV16 | bkpt1- | AGTACAGACCTACGTGACCATATAGA | 867 | AAATACGGCTGCACCGAGT | 868 |
| | MYC_001_exon2 | C | | | |
| HPV16 | bkpt1- | AGTACAGACCTACGTGACCATATAGA | 869 | GGTGATCCAGACTCTGACCT | 870 |
| | MYC_001_exon3 | C | | TTTG | |
| HPV16 | bkpt1- | AGTACAGACCTACGTGACCATATAGA | 871 | ATCATGATGGCTGTATGTGC | 872 |
| | PVT1_002_exon3 | C | | CA | |
| HPV16 | bkpt1- | AGTACAGACCTACGTGACCATATAGA | 873 | CATGGTTCCACCAGCGTTAT | 874 |
| | PVT1_004_exon1 | C | | T | |
| HPV16 | bkpt1- | AGTACAGACCTACGTGACCATATAGA | 875 | TCTTTGCTCGCAGCTCGT | 876 |
| | PVT1_005_exon1 | C | | | |
| HPV16 | bkpt2- | GCTCACACAAAGGACGGATTAAC | 877 | CTGAGAAGCCCTGCCCTTC | 878 |
| | MYC_001_exon1 | | | | |
| HPV16 | bkpt2- | GCTCACACAAAGGACGGATTAAC | 879 | AAATACGGCTGCACCGAGT | 880 |
| | MYC_001_exon2 | | | | |
| HPV16 | bkpt2- | GCTCACACAAAGGACGGATTAAC | 881 | GGTGATCCAGACTCTGACCT | 882 |
| | MYC_001_exon3 | | | TTTG | |
| HPV16 | bkpt2- | GCTCACACAAAGGACGGATTAAC | 883 | ATCATGATGGCTGTATGTGC | 884 |
| | PVT1_002_exon3 | | | CA | |
| HPV16 | bkpt2- | GCTCACACAAAGGACGGATTAAC | 885 | CATGGTTCCACCAGCGTTAT | 886 |
| | PVT1_004_exon1 | | | T | |
| HPV16 | bkpt2- | GCTCACACAAAGGACGGATTAAC | 887 | TCTTTGCTCGCAGCTCGT | 888 |
| | PVT1_005_exon1 | | | | |
| HPV16 | SD2- | GGAATTGTGTGCCCCATCTGT | 889 | CTGAGAAGCCCTGCCCTTC | 890 |
| | MYC_001_exon1 | | | | |
| HPV16 | SD2- | GGAATTGTGTGCCCCATCTGT | 891 | AAATACGGCTGCACCGAGT | 892 |
| | MYC_001_exon2 | | | | |
| HPV16 | SD2- | GGAATTGTGTGCCCCATCTGT | 893 | GGTGATCCAGACTCTGACCT | 894 |
| | MYC_001_exon3 | | | TTTG | |
| HPV16 | SD2- | GGAATTGTGTGCCCCATCTGT | 895 | ATCATGATGGCTGTATGTGC | 896 |
| | PVT1_002_exon3 | | | CA | |
| HPV16 | SD2- | GGAATTGTGTGCCCCATCTGT | 897 | CATGGTTCCACCAGCGTTAT | 898 |
| | PVT1_004_exon1 | | | T | |
| HPV16 | SD2- | GGAATTGTGTGCCCCATCTGT | 899 | TCTTTGCTCGCAGCTCGT | 900 |
| | PVT1_005_exon1 | | | | |
| HPV18 | bkpt1- | AATGACAGTAAAGACATAGACAGCC | 901 | CTGAGAAGCCCTGCCCTTC | 902 |
| | MYC_001_exon1 | AAA | | | |
| HPV18 | bkpt1- | AATGACAGTAAAGACATAGACAGCC | 903 | AAATACGGCTGCACCGAGT | 904 |
| | MYC_001_exon2 | AAA | | | |
| HPV18 | bkpt1- | AATGACAGTAAAGACATAGACAGCC | 905 | GGTGATCCAGACTCTGACCT | 906 |
| | MYC_001_exon3 | AAA | | TTTG | |
| HPV18 | bkpt1- | AATGACAGTAAAGACATAGACAGCC | 907 | ATCATGATGGCTGTATGTGC | 908 |
| | PVT1_002_exon3 | AAA | | CA | |
| HPV18 | bkpt1- | AATGACAGTAAAGACATAGACAGCC | 909 | CATGGTTCCACCAGCGTTAT | 910 |
| | PVT1_004_exon1 | AAA | | T | |
| HPV18 | bkpt1- | AATGACAGTAAAGACATAGACAGCC | 911 | TCTTTGCTCGCAGCTCGT | 912 |
| | PVT1_005_exon1 | AAA | | | |
| HPV18 | bkpt2- | CAGCTACACCTACAGGCAACAA | 913 | CTGAGAAGCCCTGCCCTTC | 914 |
| | MYC_001_exon1 | | | | |
| HPV18 | bkpt2- | CAGCTACACCTACAGGCAACAA | 915 | AAATACGGCTGCACCGAGT | 916 |
| | MYC_001_exon2 | | | | |
| HPV18 | bkpt2- | CAGCTACACCTACAGGCAACAA | 917 | GGTGATCCAGACTCTGACCT | 918 |
| | MYC_001_exon3 | | | TTTG | |
| HPV18 | bkpt2- | CAGCTACACCTACAGGCAACAA | 919 | ATCATGATGGCTGTATGTGC | 920 |
| | PVT1_002_exon3 | | | CA | |
| HPV18 | bkpt2- | CAGCTACACCTACAGGCAACAA | 921 | CATGGTTCCACCAGCGTTAT | 922 |
| | PVT1_004_exon1 | | | T | |
| HPV18 | bkpt2- | CAGCTACACCTACAGGCAACAA | 923 | TCTTTGCTCGCAGCTCGT | 924 |
| | PVT1_005_exon1 | | | | |
| HPV18 | SD2- | TGCATCCCAGCAGTAAGCAA | 925 | CTGAGAAGCCCTGCCCTTC | 926 |
| | MYC_001_exon1 | | | | |
| HPV18 | SD2- | TGCATCCCAGCAGTAAGCAA | 927 | AAATACGGCTGCACCGAGT | 928 |
| | MYC_001_exon2 | | | | |
| HPV18 | SD2- | TGCATCCCAGCAGTAAGCAA | 929 | GGTGATCCAGACTCTGACCT | 930 |
| | MYC_001_exon3 | | | TTTG | |
| HPV18 | SD2- | TGCATCCCAGCAGTAAGCAA | 931 | ATCATGATGGCTGTATGTGC | 932 |
| | PVT1_002_exon3 | | | CA | |
| HPV18 | SD2- | TGCATCCCAGCAGTAAGCAA | 933 | CATGGTTCCACCAGCGTTAT | 934 |
| | PVT1_004_exon1 | | | T | |
| HPV18 | SD2- | TGCATCCCAGCAGTAAGCAA | 935 | TCTTTGCTCGCAGCTCGT | 936 |
| | PVT1_005_exon1 | | | | |
| HPV31 | bkpt1- | CAACGTTTAAATGTGTGTCAGGACAA | 937 | CTGAGAAGCCCTGCCCTTC | 938 |
| | MYC_001_exon1 | A | | | |
| HPV31 | bkpt1- | CAACGTTTAAATGTGTGTCAGGACAA | 939 | AAATACGGCTGCACCGAGT | 940 |
| | MYC_001_exon2 | A | | | |
| HPV31 | bkpt1- | CAACGTTTAAATGTGTGTCAGGACAA | 941 | GGTGATCCAGACTCTGACCT | 942 |
| | MYC_001_exon3 | A | | TTTG | |
| HPV31 | bkpt1- | CAACGTTTAAATGTGTGTCAGGACAA | 943 | ATCATGATGGCTGTATGTGC | 944 |
| | PVT1_002_exon3 | A | | CA | |
| HPV31 | bkpt1- | CAACGTTTAAATGTGTGTCAGGACAA | 945 | CATGGTTCCACCAGCGTTAT | 946 |
| | PVT1_004_exon1 | A | | T | |
| HPV31 | bkpt1- | CAACGTTTAAATGTGTGTCAGGACAA | 947 | TCTTTGCTCGCAGCTCGT | 948 |
| | PVT1_005_exon1 | A | | | |
| HPV31 | bkpt2- | CAGCTGCATGCACAAACCA | 949 | CTGAGAAGCCCTGCCCTTC | 950 |
| | MYC_001_exon1 | | | | |
| HPV31 | bkpt2- | CAGCTGCATGCACAAACCA | 951 | AAATACGGCTGCACCGAGT | 952 |
| | MYC_001_exon2 | | | | |
| HPV31 | bkpt2- | CAGCTGCATGCACAAACCA | 953 | GGTGATCCAGACTCTGACCT | 954 |
| | MYC_001_exon3 | | | TTTG | |
| HPV31 | bkpt2- | CAGCTGCATGCACAAACCA | 955 | ATCATGATGGCTGTATGTGC | 956 |
| | PVT1_002_exon3 | | | CA | |
| HPV31 | bkpt2- | CAGCTGCATGCACAAACCA | 957 | CATGGTTCCACCAGCGTTAT | 958 |
| | PVT1_004_exon1 | | | T | |
| HPV31 | bkpt2- | CAGCTGCATGCACAAACCA | 959 | TCTTTGCTCGCAGCTCGT | 960 |
| | PVT1_005_exon1 | | | | |
| HPV31 | SD2- | AATCGTGTGCCCCAACTGT | 961 | CTGAGAAGCCCTGCCCTTC | 962 |
| | MYC_001_exon1 | | | | |
| HPV31 | SD2- | AATCGTGTGCCCCAACTGT | 963 | AAATACGGCTGCACCGAGT | 964 |
| | MYC_001_exon2 | | | | |
| HPV31 | SD2- | AATCGTGTGCCCCAACTGT | 965 | GGTGATCCAGACTCTGACCT | 966 |
| | MYC_001_exon3 | | | TTTG | |
| HPV31 | SD2- | AATCGTGTGCCCCAACTGT | 967 | ATCATGATGGCTGTATGTGC | 968 |
| | PVT1_002_exon3 | | | CA | |
| HPV31 | SD2- | AATCGTGTGCCCCAACTGT | 969 | CATGGTTCCACCAGCGTTAT | 970 |
| | PVT1_004_exon1 | | | T | |
| HPV31 | SD2- | AATCGTGTGCCCCAACTGT | 971 | TCTTTGCTCGCAGCTCGT | 972 |
| | PVT1_005_exon1 | | | | |
| HPV33 | bkpt1- | GTGCAGGAGAAAATACTAGATCTTTA | 973 | CTGAGAAGCCCTGCCCTTC | 974 |
| | MYC_001_exon1 | CGA | | | |
| HPV33 | bkpt1- | GTGCAGGAGAAAATACTAGATCTTTA | 975 | AAATACGGCTGCACCGAGT | 976 |
| | MYC_001_exon2 | CGA | | | |
| HPV33 | bkpt1- | GTGCAGGAGAAAATACTAGATCTTTA | 977 | GGTGATCCAGACTCTGACCT | 978 |
| | MYC_001_exon3 | CGA | | TTTG | |
| HPV33 | bkpt1- | GTGCAGGAGAAAATACTAGATCTTTA | 979 | ATCATGATGGCTGTATGTGC | 980 |
| | PVT1_002_exon3 | CGA | | CA | |
| HPV33 | bkpt1- | GTGCAGGAGAAAATACTAGATCTTTA | 981 | CATGGTTCCACCAGCGTTAT | 982 |
| | PVT1_004_exon1 | CGA | | T | |
| HPV33 | bkpt1- | GTGCAGGAGAAAATACTAGATCTTTA | 983 | TCTTTGCTCGCAGCTCGT | 984 |
| | PVT1_005_exon1 | CGA | | | |
| HPV33 | bkpt2- | ACGTACTGCAACTAACTGCACAA | 985 | CTGAGAAGCCCTGCCCTTC | 986 |
| | MYC_001_exon1 | | | | |
| HPV33 | bkpt2- | ACGTACTGCAACTAACTGCACAA | 987 | AAATACGGCTGCACCGAGT | 988 |
| | MYC_001_exon2 | | | | |
| HPV33 | bkpt2- | ACGTACTGCAACTAACTGCACAA | 989 | GGTGATCCAGACTCTGACCT | 990 |
| | MYC_001_exon3 | | | TTTG | |
| HPV33 | bkpt2- | ACGTACTGCAACTAACTGCACAA | 991 | ATCATGATGGCTGTATGTGC | 992 |
| | PVT1_002_exon3 | | | CA | |
| HPV33 | bkpt2- | ACGTACTGCAACTAACTGCACAA | 993 | CATGGTTCCACCAGCGTTAT | 994 |
| | PVT1_004_exon1 | | | T | |
| HPV33 | bkpt2- | ACGTACTGCAACTAACTGCACAA | 995 | TCTTTGCTCGCAGCTCGT | 996 |
| | PVT1_005_exon1 | | | | |
| HPV33 | SD2- | GTGCCCTACCTGTGCACAA | 997 | CTGAGAAGCCCTGCCCTTC | 998 |
| | MYC_001_exon1 | | | | |
| HPV33 | SD2- | GTGCCCTACCTGTGCACAA | 999 | AAATACGGCTGCACCGAGT | 1000 |
| | MYC_001_exon2 | | | | |
| HPV33 | SD2- | GTGCCCTACCTGTGCACAA | 1001 | GGTGATCCAGACTCTGACCT | 1002 |
| | MYC_001_exon3 | | | TTTG | |
| HPV33 | SD2- | GTGCCCTACCTGTGCACAA | 1003 | ATCATGATGGCTGTATGTGC | 1004 |
| | PVT1_002_exon3 | | | CA | |
| HPV33 | SD2- | GTGCCCTACCTGTGCACAA | 1005 | CATGGTTCCACCAGCGTTAT | 1006 |
| | PVT1_004_exon1 | | | T | |
| HPV33 | SD2- | GTGCCCTACCTGTGCACAA | 1007 | TCTTTGCTCGCAGCTCGT | 1008 |
| | PVT1_005_exon1 | | | | |
| HPV35 | bkpt1- | ATTACGAGACTGATAGCACATGTTTG | 1009 | CTGAGAAGCCCTGCCCTTC | 1010 |
| | MYC_001_exon1 | T | | | |
| HPV35 | bkpt1- | ATTACGAGACTGATAGCACATGTTTG | 1011 | AAATACGGCTGCACCGAGT | 1012 |
| | MYC_001_exon2 | T | | | |
| HPV35 | bkpt1- | ATTACGAGACTGATAGCACATGTTTG | 1013 | GGTGATCCAGACTCTGACCT | 1014 |
| | MYC_001_exon3 | T | | TTTG | |
| HPV35 | bkpt1- | ATTACGAGACTGATAGCACATGTTTG | 1015 | ATCATGATGGCTGTATGTGC | 1016 |
| | PVT1_002_exon3 | T | | CA | |
| HPV35 | bkpt1- | ATTACGAGACTGATAGCACATGTTTG | 1017 | CATGGTTCCACCAGCGTTAT | 1018 |
| | PVT1_004_exon1 | T | | T | |
| HPV35 | bkpt1- | ATTACGAGACTGATAGCACATGTTTG | 1019 | TCTTTGCTCGCAGCTCGT | 1020 |
| | PVT1_005_exon1 | T | | | |
| HPV35 | bkpt2- | TCTACATCTGACTGCACAAACAAAGA | 1021 | CTGAGAAGCCCTGCCCTTC | 1022 |
| | MYC_001_exon1 | | | | |
| HPV35 | bkpt2- | TCTACATCTGACTGCACAAACAAAGA | 1023 | AAATACGGCTGCACCGAGT | 1024 |
| | MYC_001_exon2 | | | | |
| HPV35 | bkpt2- | TCTACATCTGACTGCACAAACAAAGA | 1025 | GGTGATCCAGACTCTGACCT | 1026 |
| | MYC_001_exon3 | | | TTTG | |
| HPV35 | bkpt2- | TCTACATCTGACTGCACAAACAAAGA | 1027 | ATCATGATGGCTGTATGTGC | 1028 |
| | PVT1_002_exon3 | | | CA | |
| HPV35 | bkpt2- | TCTACATCTGACTGCACAAACAAAGA | 1029 | CATGGTTCCACCAGCGTTAT | 1030 |
| | PVT1_004_exon1 | | | T | |
| HPV35 | bkpt2- | TCTACATCTGACTGCACAAACAAAGA | 1031 | TCTTTGCTCGCAGCTCGT | 1032 |
| | PVT1_005_exon1 | | | | |
| HPV35 | SD2- | CGGCTGTTCACAGAGAGCATAAT | 1033 | CTGAGAAGCCCTGCCCTTC | 1034 |
| | MYC_001_exon1 | | | | |
| HPV35 | SD2- | CGGCTGTTCACAGAGAGCATAAT | 1035 | AAATACGGCTGCACCGAGT | 1036 |
| | MYC_001_exon2 | | | | |
| HPV35 | SD2- | CGGCTGTTCACAGAGAGCATAAT | 1037 | GGTGATCCAGACTCTGACCT | 1038 |
| | MYC_001_exon3 | | | TTTG | |
| HPV35 | SD2- | CGGCTGTTCACAGAGAGCATAAT | 1039 | ATCATGATGGCTGTATGTGC | 1040 |
| | PVT1_002_exon3 | | | CA | |
| HPV35 | SD2- | CGGCTGTTCACAGAGAGCATAAT | 1041 | CATGGTTCCACCAGCGTTAT | 1042 |
| | PVT1_004_exon1 | | | T | |
| HPV35 | SD2- | CGGCTGTTCACAGAGAGCATAAT | 1043 | TCTTTGCTCGCAGCTCGT | 1044 |
| | PVT1_005_exon1 | | | | |
| HPV39 | bkpt1- | ACAACGTTTAAATGTGTTACAGGACA | 1045 | CTGAGAAGCCCTGCCCTTC | 1046 |
| | MYC_001_exon1 | | | | |
| HPV39 | bkpt1- | ACAACGTTTAAATGTGTTACAGGACA | 1047 | AAATACGGCTGCACCGAGT | 1048 |
| | MYC_001_exon2 | | | | |
| HPV39 | bkpt1- | ACAACGTTTAAATGTGTTACAGGACA | 1049 | GGTGATCCAGACTCTGACCT | 1050 |
| | MYC_001_exon3 | | | TTTG | |
| HPV39 | bkpt1- | ACAACGTTTAAATGTGTTACAGGACA | 1051 | ATCATGATGGCTGTATGTGC | 1052 |
| | PVT1_002_exon3 | | | CA | |
| HPV39 | bkpt1- | ACAACGTTTAAATGTGTTACAGGACA | 1053 | CATGGTTCCACCAGCGTTAT | 1054 |
| | PVT1_004_exon1 | | | T | |
| HPV39 | bkpt1- | ACAACGTTTAAATGTGTTACAGGACA | 1055 | TCTTTGCTCGCAGCTCGT | 1056 |
| | PVT1_005_exon1 | | | | |
| HPV39 | bkpt2- | CACAGTAACAGTACAGGCCACA | 1057 | CTGAGAAGCCCTGCCCTTC | 1058 |
| | MYC_001_exon1 | | | | |
| HPV39 | bkpt2- | CACAGTAACAGTACAGGCCACA | 1059 | AAATACGGCTGCACCGAGT | 1060 |
| | MYC_001_exon2 | | | | |
| HPV39 | bkpt2- | CACAGTAACAGTACAGGCCACA | 1061 | GGTGATCCAGACTCTGACCT | 1062 |
| | MYC_001_exon3 | | | TTTG | |
| HPV39 | bkpt2- | CACAGTAACAGTACAGGCCACA | 1063 | ATCATGATGGCTGTATGTGC | 1064 |
| | PVT1_002_exon3 | | | CA | |
| HPV39 | bkpt2- | CACAGTAACAGTACAGGCCACA | 1065 | CATGGTTCCACCAGCGTTAT | 1066 |
| | PVT1_004_exon1 | | | T | |
| HPV39 | bkpt2- | CACAGTAACAGTACAGGCCACA | 1067 | TCTTTGCTCGCAGCTCGT | 1068 |
| | PVT1_005_exon1 | | | | |
| HPV39 | SD2- | CGTGGTGTGCAACTGCAA | 1069 | CTGAGAAGCCCTGCCCTTC | 1070 |
| | MYC_001_exon1 | | | | |
| HPV39 | SD2- | CGTGGTGTGCAACTGCAA | 1071 | AAATACGGCTGCACCGAGT | 1072 |
| | MYC_001_exon2 | | | | |
| HPV39 | SD2- | CGTGGTGTGCAACTGCAA | 1073 | GGTGATCCAGACTCTGACCT | 1074 |
| | MYC_001_exon3 | | | TTTG | |
| HPV39 | SD2- | CGTGGTGTGCAACTGCAA | 1075 | ATCATGATGGCTGTATGTGC | 1076 |
| | PVT1_002_exon3 | | | CA | |
| HPV39 | SD2- | CGTGGTGTGCAACTGCAA | 1077 | CATGGTTCCACCAGCGTTAT | 1078 |
| | PVT1_004_exon1 | | | T | |
| HPV39 | SD2- | CGTGGTGTGCAACTGCAA | 1079 | TCTTTGCTCGCAGCTCGT | 1080 |
| | PVT1_005_exon1 | | | | |
| HPV45 | bkpt1- | CGTTACAGGACAAAATACTAGACCAC | 1081 | CTGAGAAGCCCTGCCCTTC | 1082 |
| | MYC_001_exon1 | TA | | | |
| HPV45 | bkpt1- | CGTTACAGGACAAAATACTAGACCAC | 1083 | AAATACGGCTGCACCGAGT | 1084 |
| | MYC_001_exon2 | TA | | | |
| HPV45 | bkpt1- | CGTTACAGGACAAAATACTAGACCAC | 1085 | GGTGATCCAGACTCTGACCT | 1086 |
| | MYC_001_exon3 | TA | | TTTG | |
| HPV45 | bkpt1- | CGTTACAGGACAAAATACTAGACCAC | 1087 | ATCATGATGGCTGTATGTGC | 1088 |
| | PVT1_002_exon3 | TA | | CA | |
| HPV45 | bkpt1- | CGTTACAGGACAAAATACTAGACCAC | 1089 | CATGGTTCCACCAGCGTTAT | 1090 |
| | PVT1_004_exon1 | TA | | T | |
| HPV45 | bkpt1- | CGTTACAGGACAAAATACTAGACCAC | 1091 | TCTTTGCTCGCAGCTCGT | 1092 |
| | PVT1_005_exon1 | TA | | | |
| HPV45 | bkpt2- | TCCTGTGTTCAAGTACAAGTAACAAC | 1093 | CTGAGAAGCCCTGCCCTTC | 1094 |
| | MYC_001_exon1 | AA | | | |
| HPV45 | bkpt2- | TCCTGTGTTCAAGTACAAGTAACAAC | 1095 | AAATACGGCTGCACCGAGT | 1096 |
| | MYC_001_exon2 | AA | | | |
| HPV45 | bkpt2- | TCCTGTGTTCAAGTACAAGTAACAAC | 1097 | GGTGATCCAGACTCTGACCT | 1098 |
| | MYC_001_exon3 | AA | | TTTG | |
| HPV45 | bkpt2- | TCCTGTGTTCAAGTACAAGTAACAAC | 1099 | ATCATGATGGCTGTATGTGC | 1100 |
| | PVT1_002_exon3 | AA | | CA | |
| HPV45 | bkpt2- | TCCTGTGTTCAAGTACAAGTAACAAC | 1101 | CATGGTTCCACCAGCGTTAT | 1102 |
| | PVT1_004_exon1 | AA | | T | |
| HPV45 | bkpt2- | TCCTGTGTTCAAGTACAAGTAACAAC | 1103 | TCTTTGCTCGCAGCTCGT | 1104 |
| | PVT1_005_exon1 | AA | | | |
| HPV45 | SD2- | AGCACCTTGTCCTTTGTGTGT | 1105 | CTGAGAAGCCCTGCCCTTC | 1106 |
| | MYC_001_exon1 | | | | |
| HPV45 | SD2- | AGCACCTTGTCCTTTGTGTGT | 1107 | AAATACGGCTGCACCGAGT | 1108 |
| | MYC_001_exon2 | | | | |
| HPV45 | SD2- | AGCACCTTGTCCTTTGTGTGT | 1109 | GGTGATCCAGACTCTGACCT | 1110 |
| | MYC_001_exon3 | | | TTTG | |
| HPV45 | SD2- | AGCACCTTGTCCTTTGTGTGT | 1111 | ATCATGATGGCTGTATGTGC | 1112 |
| | PVT1_002_exon3 | | | CA | |
| HPV45 | SD2- | AGCACCTTGTCCTTTGTGTGT | 1113 | CATGGTTCCACCAGCGTTAT | 1114 |
| | PVT1_004_exon1 | | | T | |
| HPV45 | SD2- | AGCACCTTGTCCTTTGTGTGT | 1115 | TCTTTGCTCGCAGCTCGT | 1116 |
| | PVT1_005_exon1 | | | | |
| HPV51 | bkpt1- | GTGCCAGGAGAAAATACTAGACTGTT | 1117 | CTGAGAAGCCCTGCCCTTC | 1118 |
| | MYC_001_exon1 | AT | | | |
| HPV51 | bkpt1- | GTGCCAGGAGAAAATACTAGACTGTT | 1119 | AAATACGGCTGCACCGAGT | 1120 |
| | MYC_001_exon2 | AT | | | |
| HPV51 | bkpt1- | GTGCCAGGAGAAAATACTAGACTGTT | 1121 | GGTGATCCAGACTCTGACCT | 1122 |
| | MYC_001_exon3 | AT | | TTTG | |
| HPV51 | bkpt1- | GTGCCAGGAGAAAATACTAGACTGTT | 1123 | ATCATGATGGCTGTATGTGC | 1124 |
| | PVT1_002_exon3 | AT | | CA | |
| HPV51 | bkpt1- | GTGCCAGGAGAAAATACTAGACTGTT | 1125 | CATGGTTCCACCAGCGTTAT | 1126 |
| | PVT1_004_exon1 | AT | | T | |
| HPV51 | bkpt1- | GTGCCAGGAGAAAATACTAGACTGTT | 1127 | TCTTTGCTCGCAGCTCGT | 1128 |
| | PVT1_005_exon1 | AT | | | |
| HPV51 | bkpt2- | CTAACACTGGAGGGCACCAAA | 1129 | CTGAGAAGCCCTGCCCTTC | 1130 |
| | MYC_001_exon1 | | | | |
| HPV51 | bkpt2- | CTAACACTGGAGGGCACCAAA | 1131 | AAATACGGCTGCACCGAGT | 1132 |
| | MYC_001_exon2 | | | | |
| HPV51 | bkpt2- | CTAACACTGGAGGGCACCAAA | 1133 | GGTGATCCAGACTCTGACCT | 1134 |
| | MYC_001_exon3 | | | TTTG | |
| HPV51 | bkpt2- | CTAACACTGGAGGGCACCAAA | 1135 | ATCATGATGGCTGTATGTGC | 1136 |
| | PVT1_002_exon3 | | | CA | |
| HPV51 | bkpt2- | CTAACACTGGAGGGCACCAAA | 1137 | CATGGTTCCACCAGCGTTAT | 1138 |
| | PVT1_004_exon1 | | | T | |
| HPV51 | bkpt2- | CTAACACTGGAGGGCACCAAA | 1139 | TCTTTGCTCGCAGCTCGT | 1140 |
| | PVT1_005_exon1 | | | | |
| HPV51 | SD2- | GGGCGAACTAAGCCTGGTTT | 1141 | CTGAGAAGCCCTGCCCTTC | 1142 |
| | MYC_001_exon1 | | | | |
| HPV51 | SD2- | GGGCGAACTAAGCCTGGTTT | 1143 | AAATACGGCTGCACCGAGT | 1144 |
| | MYC_001_exon2 | | | | |
| HPV51 | SD2- | GGGCGAACTAAGCCTGGTTT | 1145 | GGTGATCCAGACTCTGACCT | 1146 |
| | MYC_001_exon3 | | | TTTG | |
| HPV51 | SD2- | GGGCGAACTAAGCCTGGTTT | 1147 | ATCATGATGGCTGTATGTGC | 1148 |
| | PVT1_002_exon3 | | | CA | |
| HPV51 | SD2- | GGGCGAACTAAGCCTGGTTT | 1149 | CATGGTTCCACCAGCGTTAT | 1150 |
| | PVT1_004_exon1 | | | T | |
| HPV51 | SD2- | GGGCGAACTAAGCCTGGTTT | 1151 | TCTTTGCTCGCAGCTCGT | 1152 |
| | PVT1_005_exon1 | | | | |
| HPV52 | bkpt1- | GCTGATAGTAATGACCTAAACGCACA | 1153 | CTGAGAAGCCCTGCCCTTC | 1154 |
| | MYC_001_exon1 | AA | | | |
| HPV52 | bkpt1- | GCTGATAGTAATGACCTAAACGCACA | 1155 | AAATACGGCTGCACCGAGT | 1156 |
| | MYC_001_exon2 | AA | | | |
| HPV52 | bkpt1- | GCTGATAGTAATGACCTAAACGCACA | 1157 | GGTGATCCAGACTCTGACCT | 1158 |
| | MYC_001_exon3 | AA | | TTTG | |
| HPV52 | bkpt1- | GCTGATAGTAATGACCTAAACGCACA | 1159 | ATCATGATGGCTGTATGTGC | 1160 |
| | PVT1_002_exon3 | AA | | CA | |
| HPV52 | bkpt1- | GCTGATAGTAATGACCTAAACGCACA | 1161 | CATGGTTCCACCAGCGTTAT | 1162 |
| | PVT1_004_exon1 | AA | | T | |
| HPV52 | bkpt1- | GCTGATAGTAATGACCTAAACGCACA | 1163 | TCTTTGCTCGCAGCTCGT | 1164 |
| | PVT1_005_exon1 | AA | | | |
| HPV52 | bkpt2- | TCACTGCAACTGAGTGCACAA | 1165 | CTGAGAAGCCCTGCCCTTC | 1166 |
| | MYC_001_exon1 | | | | |
| HPV52 | bkpt2- | TCACTGCAACTGAGTGCACAA | 1167 | AAATACGGCTGCACCGAGT | 1168 |
| | MYC_001_exon2 | | | | |
| HPV52 | bkpt2- | TCACTGCAACTGAGTGCACAA | 1169 | GGTGATCCAGACTCTGACCT | 1170 |
| | MYC_001_exon3 | | | TTTG | |
| HPV52 | bkpt2- | TCACTGCAACTGAGTGCACAA | 1171 | ATCATGATGGCTGTATGTGC | 1172 |
| | PVT1_002_exon3 | | | CA | |
| HPV52 | bkpt2- | TCACTGCAACTGAGTGCACAA | 1173 | CATGGTTCCACCAGCGTTAT | 1174 |
| | PVT1_004_exon1 | | | T | |
| HPV52 | bkpt2- | TCACTGCAACTGAGTGCACAA | 1175 | TCTTTGCTCGCAGCTCGT | 1176 |
| | PVT1_005_exon1 | | | | |
| HPV52 | SD2- | GCTGTTGGGCACATTACAAGTT | 1177 | CTGAGAAGCCCTGCCCTTC | 1178 |
| | MYC_001_exon1 | | | | |
| HPV52 | SD2- | GCTGTTGGGCACATTACAAGTT | 1179 | AAATACGGCTGCACCGAGT | 1180 |
| | MYC_001_exon2 | | | | |
| HPV52 | SD2- | GCTGTTGGGCACATTACAAGTT | 1181 | GGTGATCCAGACTCTGACCT | 1182 |
| | MYC_001_exon3 | | | TTTG | |
| HPV52 | SD2- | GCTGTTGGGCACATTACAAGTT | 1183 | ATCATGATGGCTGTATGTGC | 1184 |
| | PVT1_002_exon3 | | | CA | |
| HPV52 | SD2- | GCTGTTGGGCACATTACAAGTT | 1185 | CATGGTTCCACCAGCGTTAT | 1186 |
| | PVT1_004_exon1 | | | T | |
| HPV52 | SD2- | GCTGTTGGGCACATTACAAGTT | 1187 | TCTTTGCTCGCAGCTCGT | 1188 |
| | PVT1_005_exon1 | | | | |
| HPV56 | bkpt1- | GTGCCAGAACAAAATACTAGACTGTT | 1189 | CTGAGAAGCCCTGCCCTTC | 1190 |
| | MYC_001_exon1 | T | | | |
| HPV56 | bkpt1- | GTGCCAGAACAAAATACTAGACTGTT | 1191 | AAATACGGCTGCACCGAGT | 1192 |
| | MYC_001_exon2 | T | | | |
| HPV56 | bkpt1- | GTGCCAGAACAAAATACTAGACTGTT | 1193 | GGTGATCCAGACTCTGACCT | 1194 |
| | MYC_001_exon3 | T | | TTTG | |
| HPV56 | bkpt1- | GTGCCAGAACAAAATACTAGACTGTT | 1195 | ATCATGATGGCTGTATGTGC | 1196 |
| | PVT1_002_exon3 | T | | CA | |
| HPV56 | bkpt1- | GTGCCAGAACAAAATACTAGACTGTT | 1197 | CATGGTTCCACCAGCGTTAT | 1198 |
| | PVT1_004_exon1 | T | | T | |
| HPV56 | bkpt1- | GTGCCAGAACAAAATACTAGACTGTT | 1199 | TCTTTGCTCGCAGCTCGT | 1200 |
| | PVT1_005_exon1 | T | | | |
| HPV56 | bkpt2- | ACAACAACCACCCTGGTGATAAG | 1201 | CTGAGAAGCCCTGCCCTTC | 1202 |
| | MYC_001_exon1 | | | | |
| HPV56 | bkpt2- | ACAACAACCACCCTGGTGATAAG | 1203 | AAATACGGCTGCACCGAGT | 1204 |
| | MYC_001_exon2 | | | | |
| HPV56 | bkpt2- | ACAACAACCACCCTGGTGATAAG | 1205 | GGTGATCCAGACTCTGACCT | 1206 |
| | MYC_001_exon3 | | | TTTG | |
| HPV56 | bkpt2- | ACAACAACCACCCTGGTGATAAG | 1207 | ATCATGATGGCTGTATGTGC | 1208 |
| | PVT1_002_exon3 | | | CA | |
| HPV56 | bkpt2- | ACAACAACCACCCTGGTGATAAG | 1209 | CATGGTTCCACCAGCGTTAT | 1210 |
| | PVT1_004_exon1 | | | T | |
| HPV56 | bkpt2- | ACAACAACCACCCTGGTGATAAG | 1211 | TCTTTGCTCGCAGCTCGT | 1212 |
| | PVT1_005_exon1 | | | | |
| HPV56 | SD2- | GTTAACAGTAACGTGCCCACTCT | 1213 | CTGAGAAGCCCTGCCCTTC | 1214 |
| | MYC_001_exon1 | | | | |
| HPV56 | SD2- | GTTAACAGTAACGTGCCCACTCT | 1215 | AAATACGGCTGCACCGAGT | 1216 |
| | MYC_001_exon2 | | | | |
| HPV56 | SD2- | GTTAACAGTAACGTGCCCACTCT | 1217 | GGTGATCCAGACTCTGACCT | 1218 |
| | MYC_001_exon3 | | | TTTG | |
| HPV56 | SD2- | GTTAACAGTAACGTGCCCACTCT | 1219 | ATCATGATGGCTGTATGTGC | 1220 |
| | PVT1_002_exon3 | | | CA | |
| HPV56 | SD2- | GTTAACAGTAACGTGCCCACTCT | 1221 | CATGGTTCCACCAGCGTTAT | 1222 |
| | PVT1_004_exon1 | | | T | |
| HPV56 | SD2- | GTTAACAGTAACGTGCCCACTCT | 1223 | TCTTTGCTCGCAGCTCGT | 1224 |
| | PVT1_005_exon1 | | | | |
| HPV58 | bkpt1- | GCAGGACAAAATCCTAGACATATAC | 1225 | CTGAGAAGCCCTGCCCTTC | 1226 |
| | MYC_001_exon1 | GAA | | | |
| HPV58 | bkpt1- | GCAGGACAAAATCCTAGACATATAC | 1227 | AAATACGGCTGCACCGAGT | 1228 |
| | MYC_001_exon2 | GAA | | | |
| HPV58 | bkpt1- | GCAGGACAAAATCCTAGACATATAC | 1229 | GGTGATCCAGACTCTGACCT | 1230 |
| | MYC_001_exon3 | GAA | | TTTG | |
| HPV58 | bkpt1- | GCAGGACAAAATCCTAGACATATAC | 1231 | ATCATGATGGCTGTATGTGC | 1232 |
| | PVT1_002_exon3 | GAA | | CA | |
| HPV58 | bkpt1- | GCAGGACAAAATCCTAGACATATAC | 1233 | CATGGTTCCACCAGCGTTAT | 1234 |
| | PVT1_004_exon1 | GAA | | T | |
| HPV58 | bkpt1- | GCAGGACAAAATCCTAGACATATAC | 1235 | TCTTTGCTCGCAGCTCGT | 1236 |
| | PVT1_005_exon1 | GAA | | | |
| HPV58 | bkpt2- | GAGGAGGACTACACAGTACAACTAA | 1237 | CTGAGAAGCCCTGCCCTTC | 1238 |
| | MYC_001_exon1 | CT | | | |
| HPV58 | bkpt2- | GAGGAGGACTACACAGTACAACTAA | 1239 | AAATACGGCTGCACCGAGT | 1240 |
| | MYC_001_exon2 | CT | | | |
| HPV58 | bkpt2- | GAGGAGGACTACACAGTACAACTAA | 1241 | GGTGATCCAGACTCTGACCT | 1242 |
| | MYC_001_exon3 | CT | | TTTG | |
| HPV58 | bkpt2- | GAGGAGGACTACACAGTACAACTAA | 1243 | ATCATGATGGCTGTATGTGC | 1244 |
| | PVT1_002_exon3 | CT | | CA | |
| HPV58 | bkpt2- | GAGGAGGACTACACAGTACAACTAA | 1245 | CATGGTTCCACCAGCGTTAT | 1246 |
| | PVT1_004_exon1 | CT | | T | |
| HPV58 | bkpt2- | GAGGAGGACTACACAGTACAACTAA | 1247 | TCTTTGCTCGCAGCTCGT | 1248 |
| | PVT1_005_exon1 | CT | | | |
| HPV58 | SD2- | TGCTTATGGGCACATGTACCATT | 1249 | CTGAGAAGCCCTGCCCTTC | 1250 |
| | MYC_001_exon1 | | | | |
| HPV58 | SD2- | TGCTTATGGGCACATGTACCATT | 1251 | AAATACGGCTGCACCGAGT | 1252 |
| | MYC_001_exon2 | | | | |
| HPV58 | SD2- | TGCTTATGGGCACATGTACCATT | 1253 | GGTGATCCAGACTCTGACCT | 1254 |
| | MYC_001_exon3 | | | TTTG | |
| HPV58 | SD2- | TGCTTATGGGCACATGTACCATT | 1255 | ATCATGATGGCTGTATGTGC | 1256 |
| | PVT1_002_exon3 | | | CA | |
| HPV58 | SD2- | TGCTTATGGGCACATGTACCATT | 1257 | CATGGTTCCACCAGCGTTAT | 1258 |
| | PVT1_004_exon1 | | | T | |
| HPV58 | SD2- | TGCTTATGGGCACATGTACCATT | 1259 | TCTTTGCTCGCAGCTCGT | 1260 |
| | PVT1_005_exon1 | | | | |
| HPV59 | bkpt1- | GCGTTTAAGTGTGTTACAGGATCAAA | 1261 | CTGAGAAGCCCTGCCCTTC | 1262 |
| | MYC_001_exon1 | T | | | |
| HPV59 | bkpt1- | GCGTTTAAGTGTGTTACAGGATCAAA | 1263 | AAATACGGCTGCACCGAGT | 1264 |
| | MYC_001_exon2 | T | | | |
| HPV59 | bkpt1- | GCGTTTAAGTGTGTTACAGGATCAAA | 1265 | GGTGATCCAGACTCTGACCT | 1266 |
| | MYC_001_exon3 | T | | TTTG | |
| HPV59 | bkpt1- | GCGTTTAAGTGTGTTACAGGATCAAA | 1267 | ATCATGATGGCTGTATGTGC | 1268 |
| | PVT1_002_exon3 | T | | CA | |
| HPV59 | bkpt1- | GCGTTTAAGTGTGTTACAGGATCAAA | 1269 | CATGGTTCCACCAGCGTTAT | 1270 |
| | PVT1_004_exon1 | T | | T | |
| HPV59 | bkpt1- | GCGTTTAAGTGTGTTACAGGATCAAA | 1271 | TCTTTGCTCGCAGCTCGT | 1272 |
| | PVT1_005_exon1 | T | | | |
| HPV59 | bkpt2- | TCCGTTTGCATCCAGGCAA | 1273 | CTGAGAAGCCCTGCCCTTC | 1274 |
| | MYC_001_exon1 | | | | |
| HPV59 | bkpt2- | TCCGTTTGCATCCAGGCAA | 1275 | AAATACGGCTGCACCGAGT | 1276 |
| | MYC_001_exon2 | | | | |
| HPV59 | bkpt2- | TCCGTTTGCATCCAGGCAA | 1277 | GGTGATCCAGACTCTGACCT | 1278 |
| | MYC_001_exon3 | | | TTTG | |
| HPV59 | bkpt2- | TCCGTTTGCATCCAGGCAA | 1279 | ATCATGATGGCTGTATGTGC | 1280 |
| | PVT1_002_exon3 | | | CA | |
| HPV59 | bkpt2- | TCCGTTTGCATCCAGGCAA | 1281 | CATGGTTCCACCAGCGTTAT | 1282 |
| | PVT1_004_exon1 | | | T | |
| HPV59 | bkpt2- | TCCGTTTGCATCCAGGCAA | 1283 | TCTTTGCTCGCAGCTCGT | 1284 |
| | PVT1_005_exon1 | | | | |
| HPV59 | SD2- | ACTATCCTTTGTGTGTCCTTTGTGT | 1285 | CTGAGAAGCCCTGCCCTTC | 1286 |
| | MYC_001_exon1 | | | | |
| HPV59 | SD2- | ACTATCCTTTGTGTGTCCTTTGTGT | 1287 | AAATACGGCTGCACCGAGT | 1288 |
| | MYC_001_exon2 | | | | |
| HPV59 | SD2- | ACTATCCTTTGTGTGTCCTTTGTGT | 1289 | GGTGATCCAGACTCTGACCT | 1290 |
| | MYC_001_exon3 | | | TTTG | |
| HPV59 | SD2- | ACTATCCTTTGTGTGTCCTTTGTGT | 1291 | ATCATGATGGCTGTATGTGC | 1292 |
| | PVT1_002_exon3 | | | CA | |
| HPV59 | SD2- | ACTATCCTTTGTGTGTCCTTTGTGT | 1293 | CATGGTTCCACCAGCGTTAT | 1294 |
| | PVT1_004_exon1 | | | T | |
| HPV59 | SD2- | ACTATCCTTTGTGTGTCCTTTGTGT | 1295 | TCTTTGCTCGCAGCTCGT | 1296 |
| | PVT1_005_exon1 | | | | |
| HPV66 | bkpt1- | CGTGCCAGAACAAAATACTAGACTGT | 1297 | CTGAGAAGCCCTGCCCTTC | 1298 |
| | MYC_001_exon1 | | | | |
| HPV66 | bkpt1- | CGTGCCAGAACAAAATACTAGACTGT | 1299 | AAATACGGCTGCACCGAGT | 1300 |
| | MYC_001_exon2 | | | | |
| HPV66 | bkpt1- | CGTGCCAGAACAAAATACTAGACTGT | 1301 | GGTGATCCAGACTCTGACCT | 1302 |
| | MYC_001_exon3 | | | TTTG | |
| HPV66 | bkpt1- | CGTGCCAGAACAAAATACTAGACTGT | 1303 | ATCATGATGGCTGTATGTGC | 1304 |
| | PVT1_002_exon3 | | | CA | |
| HPV66 | bkpt1- | CGTGCCAGAACAAAATACTAGACTGT | 1305 | CATGGTTCCACCAGCGTTAT | 1306 |
| | PVT1_004_exon1 | | | T | |
| HPV66 | bkpt1- | CGTGCCAGAACAAAATACTAGACTGT | 1307 | TCTTTGCTCGCAGCTCGT | 1308 |
| | PVT1_005_exon1 | | | | |
| HPV66 | bkpt2- | GTATCAACACACAAAGCCACTGT | 1309 | CTGAGAAGCCCTGCCCTTC | 1310 |
| | MYC_001_exon1 | | | | |
| HPV66 | bkpt2- | GTATCAACACACAAAGCCACTGT | 1311 | AAATACGGCTGCACCGAGT | 1312 |
| | MYC_001_exon2 | | | | |
| HPV66 | bkpt2- | GTATCAACACACAAAGCCACTGT | 1313 | GGTGATCCAGACTCTGACCT | 1314 |
| | MYC_001_exon3 | | | TTTG | |
| HPV66 | bkpt2- | GTATCAACACACAAAGCCACTGT | 1315 | ATCATGATGGCTGTATGTGC | 1316 |
| | PVT1_002_exon3 | | | CA | |
| HPV66 | bkpt2- | GTATCAACACACAAAGCCACTGT | 1317 | CATGGTTCCACCAGCGTTAT | 1318 |
| | PVT1_004_exon1 | | | T | |
| HPV66 | bkpt2- | GTATCAACACACAAAGCCACTGT | 1319 | TCTTTGCTCGCAGCTCGT | 1320 |
| | PVT1_005_exon1 | | | | |
| HPV66 | SD2- | GTTAACAGTAACGTGCCCACTCT | 1321 | CTGAGAAGCCCTGCCCTTC | 1322 |
| | MYC_001_exon1 | | | | |
| HPV66 | SD2- | GTTAACAGTAACGTGCCCACTCT | 1323 | AAATACGGCTGCACCGAGT | 1324 |
| | MYC_001_exon2 | | | | |
| HPV66 | SD2- | GTTAACAGTAACGTGCCCACTCT | 1325 | GGTGATCCAGACTCTGACCT | 1326 |
| | MYC_001_exon3 | | | TTTG | |
| HPV66 | SD2- | GTTAACAGTAACGTGCCCACTCT | 1327 | ATCATGATGGCTGTATGTGC | 1328 |
| | PVT1_002_exon3 | | | CA | |
| HPV66 | SD2- | GTTAACAGTAACGTGCCCACTCT | 1329 | CATGGTTCCACCAGCGTTAT | 1330 |
| | PVT1_004_exon1 | | | T | |
| HPV66 | SD2- | GTTAACAGTAACGTGCCCACTCT | 1331 | TCTTTGCTCGCAGCTCGT | 1332 |
| | PVT1_005_exon1 | | | | |
| HPV68 | bkpt1- | ACAGGACAGTAAATGTATACAGGAC | 1333 | CTGAGAAGCCCTGCCCTTC | 1334 |
| | MYC_001_exon1 | CAT | | | |
| HPV68 | bkpt1- | ACAGGACAGTAAATGTATACAGGAC | 1335 | AAATACGGCTGCACCGAGT | 1336 |
| | MYC_001_exon2 | CAT | | | |
| HPV68 | bkpt1- | ACAGGACAGTAAATGTATACAGGAC | 1337 | GGTGATCCAGACTCTGACCT | 1338 |
| | MYC_001_exon3 | CAT | | TTTG | |
| HPV68 | bkpt1- | ACAGGACAGTAAATGTATACAGGAC | 1339 | ATCATGATGGCTGTATGTGC | 1340 |
| | PVT1_002_exon3 | CAT | | CA | |
| HPV68 | bkpt1- | ACAGGACAGTAAATGTATACAGGAC | 1341 | CATGGTTCCACCAGCGTTAT | 1342 |
| | PVT1_004_exon1 | CAT | | T | |
| HPV68 | bkpt1- | ACAGGACAGTAAATGTATACAGGAC | 1343 | TCTTTGCTCGCAGCTCGT | 1344 |
| | PVT1_005_exon1 | CAT | | | |
| HPV68 | bkpt2- | AGTAGAAGTGCAGGCCAAAACAA | 1345 | CTGAGAAGCCCTGCCCTTC | 1346 |
| | MYC_001_exon1 | | | | |
| HPV68 | bkpt2- | AGTAGAAGTGCAGGCCAAAACAA | 1347 | AAATACGGCTGCACCGAGT | 1348 |
| | MYC_001_exon2 | | | | |
| HPV68 | bkpt2- | AGTAGAAGTGCAGGCCAAAACAA | 1349 | GGTGATCCAGACTCTGACCT | 1350 |
| | MYC_001_exon3 | | | TTTG | |
| HPV68 | bkpt2- | AGTAGAAGTGCAGGCCAAAACAA | 1351 | ATCATGATGGCTGTATGTGC | 1352 |
| | PVT1_002_exon3 | | | CA | |
| HPV68 | bkpt2- | AGTAGAAGTGCAGGCCAAAACAA | 1353 | CATGGTTCCACCAGCGTTAT | 1354 |
| | PVT1_004_exon1 | | | T | |
| HPV68 | bkpt2- | AGTAGAAGTGCAGGCCAAAACAA | 1355 | TCTTTGCTCGCAGCTCGT | 1356 |
| | PVT1_005_exon1 | | | | |
| HPV68 | SD2- | TCCGTGGTGTGCAACTGAA | 1357 | CTGAGAAGCCCTGCCCTTC | 1358 |
| | MYC_001_exon1 | | | | |
| HPV68 | SD2- | TCCGTGGTGTGCAACTGAA | 1359 | AAATACGGCTGCACCGAGT | 1360 |
| | MYC_001_exon2 | | | | |
| HPV68 | SD2- | TCCGTGGTGTGCAACTGAA | 1361 | GGTGATCCAGACTCTGACCT | 1362 |
| | MYC_001_exon3 | | | TTTG | |
| HPV68 | SD2- | TCCGTGGTGTGCAACTGAA | 1363 | ATCATGATGGCTGTATGTGC | 1364 |
| | PVT1_002_exon3 | | | CA | |
| HPV68 | SD2- | TCCGTGGTGTGCAACTGAA | 1365 | CATGGTTCCACCAGCGTTAT | 1366 |
| | PVT1_004_exon1 | | | T | |
| HPV68 | SD2- | TCCGTGGTGTGCAACTGAA | 1367 | TCTTTGCTCGCAGCTCGT | 1368 |
| | PVT1_005_exon1 | | | | |
| HPV73 | bkpt1- | GTATGAACGTGACAGTGTACACCTAA | 1369 | CTGAGAAGCCCTGCCCTTC | 1370 |
| | MYC_001_exon1 | | | | |
| HPV73 | bkpt1- | GTATGAACGTGACAGTGTACACCTAA | 1371 | AAATACGGCTGCACCGAGT | 1372 |
| | MYC_001_exon2 | | | | |
| HPV73 | bkpt1- | GTATGAACGTGACAGTGTACACCTAA | 1373 | GGTGATCCAGACTCTGACCT | 1374 |
| | MYC_001_exon3 | | | TTTG | |
| HPV73 | bkpt1- | GTATGAACGTGACAGTGTACACCTAA | 1375 | ATCATGATGGCTGTATGTGC | 1376 |
| | PVT1_002_exon3 | | | CA | |
| HPV73 | bkpt1- | GTATGAACGTGACAGTGTACACCTAA | 1377 | CATGGTTCCACCAGCGTTAT | 1378 |
| | PVT1_004_exon1 | | | T | |
| HPV73 | bkpt1- | GTATGAACGTGACAGTGTACACCTAA | 1379 | TCTTTGCTCGCAGCTCGT | 1380 |
| | PVT1_005_exon1 | | | | |
| HPV73 | bkpt2- | ACCTACATCCCACCACAGAGT | 1381 | CTGAGAAGCCCTGCCCTTC | 1382 |
| | MYC_001_exon1 | | | | |
| HPV73 | bkpt2- | ACCTACATCCCACCACAGAGT | 1383 | AAATACGGCTGCACCGAGT | 1384 |
| | MYC_001_exon2 | | | | |
| HPV73 | bkpt2- | ACCTACATCCCACCACAGAGT | 1385 | GGTGATCCAGACTCTGACCT | 1386 |
| | MYC_001_exon3 | | | TTTG | |
| HPV73 | bkpt2- | ACCTACATCCCACCACAGAGT | 1387 | ATCATGATGGCTGTATGTGC | 1388 |
| | PVT1_002_exon3 | | | CA | |
| HPV73 | bkpt2- | ACCTACATCCCACCACAGAGT | 1389 | CATGGTTCCACCAGCGTTAT | 1390 |
| | PVT1_004_exon1 | | | T | |
| HPV73 | bkpt2- | ACCTACATCCCACCACAGAGT | 1391 | TCTTTGCTCGCAGCTCGT | 1392 |
| | PVT1_005_exon1 | | | | |
| HPV73 | SD2- | TGCTTATGGGTACACTAGGTATTGTG | 1393 | CTGAGAAGCCCTGCCCTTC | 1394 |
| | MYC_001_exon1 | T | | | |
| HPV73 | SD2- | TGCTTATGGGTACACTAGGTATTGTG | 1395 | AAATACGGCTGCACCGAGT | 1396 |
| | MYC_001_exon2 | T | | | |
| HPV73 | SD2- | TGCTTATGGGTACACTAGGTATTGTG | 1397 | GGTGATCCAGACTCTGACCT | 1398 |
| | MYC_001_exon3 | T | | TTTG | |
| HPV73 | SD2- | TGCTTATGGGTACACTAGGTATTGTG | 1399 | ATCATGATGGCTGTATGTGC | 1400 |
| | PVT1_002_exon3 | T | | CA | |
| HPV73 | SD2- | TGCTTATGGGTACACTAGGTATTGTG | 1401 | CATGGTTCCACCAGCGTTAT | 1402 |
| | PVT1_004_exon1 | T | | T | |
| HPV73 | SD2- | TGCTTATGGGTACACTAGGTATTGTG | 1403 | TCTTTGCTCGCAGCTCGT | 1404 |
| | PVT1_005_exon1 | T | | | |
| HPV82 | bkpt1- | GTGCCAGGAGAAAATACTAGACTGTT | 1405 | CTGAGAAGCCCTGCCCTTC | 1406 |
| | MYC_001_exon1 | AT | | | |
| HPV82 | bkpt1- | GTGCCAGGAGAAAATACTAGACTGTT | 1407 | AAATACGGCTGCACCGAGT | 1408 |
| | MYC_001_exon2 | AT | | | |
| HPV82 | bkpt1- | GTGCCAGGAGAAAATACTAGACTGTT | 1409 | GGTGATCCAGACTCTGACCT | 1410 |
| | MYC_001_exon3 | AT | | TTTG | |
| HPV82 | bkpt1- | GTGCCAGGAGAAAATACTAGACTGTT | 1411 | ATCATGATGGCTGTATGTGC | 1412 |
| | PVT1_002_exon3 | AT | | CA | |
| HPV82 | bkpt1- | GTGCCAGGAGAAAATACTAGACTGTT | 1413 | CATGGTTCCACCAGCGTTAT | 1414 |
| | PVT1_004_exon1 | AT | | T | |
| HPV82 | bkpt1- | GTGCCAGGAGAAAATACTAGACTGTT | 1415 | TCTTTGCTCGCAGCTCGT | 1416 |
| | PVT1_005_exon1 | AT | | | |
| HPV82 | bkpt2- | TGCGACCACCAAATACACTGT | 1417 | CTGAGAAGCCCTGCCCTTC | 1418 |
| | MYC_001_exon1 | | | | |
| HPV82 | bkpt2- | TGCGACCACCAAATACACTGT | 1419 | AAATACGGCTGCACCGAGT | 1420 |
| | MYC_001_exon2 | | | | |
| HPV82 | bkpt2- | TGCGACCACCAAATACACTGT | 1421 | GGTGATCCAGACTCTGACCT | 1422 |
| | MYC_001_exon3 | | | TTTG | |
| HPV82 | bkpt2- | TGCGACCACCAAATACACTGT | 1423 | ATCATGATGGCTGTATGTGC | 1424 |
| | PVT1_002_exon3 | | | CA | |
| HPV82 | bkpt2- | TGCGACCACCAAATACACTGT | 1425 | CATGGTTCCACCAGCGTTAT | 1426 |
| | PVT1_004_exon1 | | | T | |
| HPV82 | bkpt2- | TGCGACCACCAAATACACTGT | 1427 | TCTTTGCTCGCAGCTCGT | 1428 |
| | PVT1_005_exon1 | | | | |
| HPV82 | SD2- | CGTGGTGTGCGACCAACTAA | 1429 | CTGAGAAGCCCTGCCCTTC | 1430 |
| | MYC_001_exon1 | | | | |
| HPV82 | SD2- | CGTGGTGTGCGACCAACTAA | 1431 | AAATACGGCTGCACCGAGT | 1432 |
| | MYC_001_exon2 | | | | |
| HPV82 | SD2- | CGTGGTGTGCGACCAACTAA | 1433 | GGTGATCCAGACTCTGACCT | 1434 |
| | MYC_001_exon3 | | | TTTG | |
| HPV82 | SD2- | CGTGGTGTGCGACCAACTAA | 1435 | ATCATGATGGCTGTATGTGC | 1436 |
| | PVT1_002_exon3 | | | CA | |
| HPV82 | SD2- | CGTGGTGTGCGACCAACTAA | 1437 | CATGGTTCCACCAGCGTTAT | 1438 |
| | PVT1_004_exon1 | | | T | |
| HPV82 | SD2- | CGTGGTGTGCGACCAACTAA | 1439 | TCTTTGCTCGCAGCTCGT | 1440 |
| | PVT1_005_exon1 | | | | |

TABLE 2Dbis

| | | Forward | Reverse | | |
|---|---|---|---|---|---|
| | | primer | primer | | Amplicon |
| HPV | | SEQ | SEQ | Amplicon | SEQ |
| type | Region name | ID NO | ID NO | nucleic acid sequence | ID NO |
| HPV16 | bkpt1- | 865 | 866 | TATTGGAAACACATGCGCCTAGCTGCTCGCGGCCG | 1933 |
| | MYC_001_exon1 | | | CCACCGCCGGGCCCCGGCCGTCCCTGGCTCCCCTC | |
| | | | | CTGCCTCGA | |
| HPV16 | bkpt1- | 867 | 868 | TATTGGAAACACATGCGCCTAGCAGCCTCCCGCG | 1934 |
| | MYC_001_exon2 | | | ACGATGCCCCTCAACGTTAGCTTCACCAACAGGA | |
| | | | | ACTATGACCTCGACTACG | |
| HPV16 | bkpt1- | 869 | 870 | TATTGGAAACACATGCGCCTAGAGGAGGAACAAG | 1935 |
| | MYC_001_exon3 | | | AAGATGAGGAAGAAATCGATGTTGTTTCTGTGGA | |
| | | | | AAAGAGGCAGGCTCCTGG | |
| HPV16 | bkpt1- | 871 | 872 | TATTGGAAACACATGCGCCTAGCTGACCATACTCC | 1936 |
| | PVT1_002_exon3 | | | CTGGAGCCTTCTCCCGAGGTGCGCGGGTGACCT | |
| HPV16 | bkpt1- | 873 | 874 | TATTGGAAACACATGCGCCTAGTCTGAGCCTGATG | 1937 |
| | PVT1_004_exon1 | | | GATTTACAGTGATCTTCAGTGGTCTGGGG | |
| HPV16 | bkpt1- | 875 | 876 | TATTGGAAACACATGCGCCTAGCTCCGGGCAGAG | 1938 |
| | PVT1_005_exon1 | | | CGCGTGTGGCGGCCGAGCACATGGGCCCGCGGGC | |
| | | | | CGGGCGGGCTCGGGGCGGCCGGGACGAGGAGGG | |
| | | | | GCGACG | |
| HPV16 | bkpt2- | 877 | 878 | TGTAATAGTAACACTACACCCATAGCTGCTCGCGG | 1939 |
| | MYC_001_exon1 | | | CCGCCACCGCCGGGCCCCGGCCGTCCCTGGCTCCC | |
| | | | | CTCCTGCCTCGA | |
| HPV16 | bkpt2- | 879 | 880 | TGTAATAGTAACACTACACCCATAGCAGCCTCCCG | 1940 |
| | MYC_001_exon2 | | | CGACGATGCCCCTCAACGTTAGCTTCACCAACAGG | |
| | | | | AACTATGACCTCGACTACG | |
| HPV16 | bkpt2- | 881 | 882 | TGTAATAGTAACACTACACCCATAGAGGAGGAAC | 1941 |
| | MYC_001_exon3 | | | AAGAAGATGAGGAAGAAATCGATGTTGTTTCTGT | |
| | | | | GGAAAAGAGGCAGGCTCCTGG | |
| HPV16 | bkpt2- | 883 | 884 | TGTAATAGTAACACTACACCCATAGCTGACCATAC | 1942 |
| | PVT1_002_exon3 | | | TCCCTGGAGCCTTCTCCCGAGGTGCGCGGGTGACC | |
| | | | | T | |
| HPV16 | bkpt2- | 885 | 886 | TGTAATAGTAACACTACACCCATAGTCTGAGCCTG | 1943 |
| | PVT1_004_exon1 | | | ATGGATTTACAGTGATCTTCAGTGGTCTGGGG | |
| HPV16 | bkpt2- | 887 | 888 | TGTAATAGTAACACTACACCCATAGCTCCGGGCA | 1944 |
| | PVT1_005_exon1 | | | GAGCGCGTGTGGCGGCCGAGCACATGGGCCCGCG | |
| | | | | GGCCGGGCGGGCTCGGGGCGGCCGGGACGAGGA | |
| | | | | GGGGCGACG | |
| HPV16 | SD2- | 889 | 890 | TCTCAGAAACCATAATCTACCATGGCTGATCCTGC | 1945 |
| | MYC_001_exon1 | | | AGCTGCTCGCGGCCGCCACCGCCGGGCCCCGGCC | |
| | | | | GTCCCTGGCTCCCCTCCTGCCTCGA | |
| HPV16 | SD2- | 891 | 892 | TCTCAGAAACCATAATCTACCATGGCTGATCCTGC | 1946 |
| | MYC_001_exon2 | | | AGCAGCCTCCCGCGACGATGCCCCTCAACGTTAGC | |
| | | | | TTCACCAACAGGAACTATGACCTCGACTACG | |
| HPV16 | SD2- | 893 | 894 | TCTCAGAAACCATAATCTACCATGGCTGATCCTGC | 1947 |
| | MYC_001_exon3 | | | AGAGGAGGAACAAGAAGATGAGGAAGAAATCGA | |
| | | | | TGTTGTTTCTGTGGAAAAGAGGCAGGCTCCTGG | |
| HPV16 | SD2- | 895 | 896 | TCTCAGAAACCATAATCTACCATGGCTGATCCTGC | 1948 |
| | PVT1_002_exon3 | | | AGCTGACCATACTCCCTGGAGCCTTCTCCCGAGGT | |
| | | | | GCGCGGGTGACCT | |
| HPV16 | SD2- | 897 | 898 | TCTCAGAAACCATAATCTACCATGGCTGATCCTGC | 1949 |
| | PVT1_004_exon1 | | | AGTCTGAGCCTGATGGATTTACAGTGATCTTCAGT | |
| | | | | GGTCTGGGG | |
| HPV16 | SD2- | 899 | 900 | TCTCAGAAACCATAATCTACCATGGCTGATCCTGC | 1950 |
| | PVT1_005_exon1 | | | AGCTCCGGGCAGAGCGCGTGTGGCGGCCGAGCAC | |
| | | | | ATGGGCCCGCGGGCCGGGCGGGCTCGGGGCGGCC | |
| | | | | GGGACGAGGAGGGGCGACG | |
| HPV18 | bkpt1- | 901 | 902 | TACAGTATTGGCAACTAATACGTTGGGCTGCTCGC | 1951 |
| | MYC_001_exon1 | | | GGCCGCCACCGCCGGGCCCCGGCCGTCCCTGGCTC | |
| | | | | CCCTCCTGCCTCGA | |
| HPV18 | bkpt1- | 903 | 904 | TACAGTATTGGCAACTAATACGTTGGGCAGCCTCC | 1952 |
| | MYC_001_exon2 | | | CGCGACGATGCCCCTCAACGTTAGCTTCACCAACA | |
| | | | | GGAACTATGACCTCGACTACG | |
| HPV18 | bkpt1- | 905 | 906 | TACAGTATTGGCAACTAATACGTTGGGAGGAGGA | 1953 |
| | MYC_001_exon3 | | | ACAAGAAGATGAGGAAGAAATCGATGTTGTTTCT | |
| | | | | GTGGAAAAGAGGCAGGCTCCTGG | |
| HPV18 | bkpt1- | 907 | 908 | TACAGTATTGGCAACTAATACGTTGGGCTGACCAT | 1954 |
| | PVT1_002_exon3 | | | ACTCCCTGGAGCCTTCTCCCGAGGTGCGCGGGTGA | |
| | | | | CCT | |
| HPV18 | bkpt1- | 909 | 910 | TACAGTATTGGCAACTAATACGTTGGGTCTGAGCC | 1955 |
| | PVT1_004_exon1 | | | TGATGGATTTACAGTGATCTTCAGTGGTCTGGGG | |
| HPV18 | bkpt1- | 911 | 912 | TACAGTATTGGCAACTAATACGTTGGGCTCCGGGC | 1956 |
| | PVT1_005_exon1 | | | AGAGCGCGTGTGGCGGCCGAGCACATGGGCCCGC | |
| | | | | GGGCCGGGCGGGCTCGGGGCGGCCGGGACGAGG | |
| | | | | AGGGGCGACG | |
| HPV18 | bkpt2- | 913 | 914 | CAAAAGACGGAAACTCTGTAGTGGTAACACTACG | 1957 |
| | MYC_001_exon1 | | | CCTATAACTGCTCGCGGCCGCCACCGCCGGGCCCC | |
| | | | | GGCCGTCCCTGGCTCCCCTCCTGCCTCGA | |
| HPV18 | bkpt2- | 915 | 916 | CAAAAGACGGAAACTCTGTAGTGGTAACACTACG | 1958 |
| | MYC_001_exon2 | | | CCTATAACAGCCTCCCGCGACGATGCCCCTCAACG | |
| | | | | TTAGCTTCACCAACAGGAACTATGACCTCGACTAC | |
| | | | | G | |
| HPV18 | bkpt2- | 917 | 918 | CAAAAGACGGAAACTCTGTAGTGGTAACACTACG | 1959 |
| | MYC_001_exon3 | | | CCTATAAAGGAGGAACAAGAAGATGAGGAAGAA | |
| | | | | ATCGATGTTGTTTCTGTGGAAAAGAGGCAGGCTCC | |
| | | | | TGG | |
| HPV18 | bkpt2- | 919 | 920 | CAAAAGACGGAAACTCTGTAGTGGTAACACTACG | 1960 |
| | PVT1_002_exon3 | | | CCTATAACTGACCATACTCCCTGGAGCCTTCTCCC | |
| | | | | GAGGTGCGCGGGTGACCT | |
| HPV18 | bkpt2- | 921 | 922 | CAAAAGACGGAAACTCTGTAGTGGTAACACTACG | 1961 |
| | PVT1_004_exon1 | | | CCTATAATCTGAGCCTGATGGATTTACAGTGATCT | |
| | | | | TCAGTGGTCTGGGG | |
| HPV18 | bkpt2- | 923 | 924 | CAAAAGACGGAAACTCTGTAGTGGTAACACTACG | 1962 |
| | PVT1_005_exon1 | | | CCTATAACTCCGGGCAGAGCGCGTGTGGCGGCCG | |
| | | | | AGCACATGGGCCCGCGGGCCGGGCGGGCTCGGGG | |
| | | | | CGGCCGGGACGAGGAGGGGCGACG | |
| HPV18 | SD2- | 925 | 926 | CAATGGCTGATCCAGAAGCTGCTCGCGGCCGCCA | 1963 |
| | MYC_001_exon1 | | | CCGCCGGGCCCCGGCCGTCCCTGGCTCCCCTCCTG | |
| | | | | CCTCGA | |
| HPV18 | SD2- | 927 | 928 | CAATGGCTGATCCAGAAGCAGCCTCCCGCGACGA | 1964 |
| | MYC_001_exon2 | | | TGCCCCTCAACGTTAGCTTCACCAACAGGAACTAT | |
| | | | | GACCTCGACTACG | |
| HPV18 | SD2- | 929 | 930 | CAATGGCTGATCCAGAAGAGGAGGAACAAGAAG | 1965 |
| | MYC_001_exon3 | | | ATGAGGAAGAAATCGATGTTGTTTCTGTGGAAAA | |
| | | | | GAGGCAGGCTCCTGG | |
| HPV18 | SD2- | 931 | 932 | CAATGGCTGATCCAGAAGCTGACCATACTCCCTGG | 1966 |
| | PVT1_002_exon3 | | | AGCCTTCTCCCGAGGTGCGCGGGTGACCT | |
| HPV18 | SD2- | 933 | 934 | CAATGGCTGATCCAGAAGTCTGAGCCTGATGGATT | 1967 |
| | PVT1_004_exon1 | | | TACAGTGATCTTCAGTGGTCTGGGG | |
| HPV18 | SD2- | 935 | 936 | CAATGGCTGATCCAGAAGCTCCGGGCAGAGCGCG | 1968 |
| | PVT1_005_exon1 | | | TGTGGCGGCCGAGCACATGGGCCCGCGGGCCGGG | |
| | | | | CGGGCTCGGGGCGGCCGGGACGAGGAGGGGCGA | |
| | | | | CG | |
| HPV31 | bkpt1- | 937 | 938 | ATATTAGAACATTATGAAAATGATAGTAAACGAC | 1969 |
| | MYC_001_exon1 | | | TTTGTGATCATATAGACTATTGGAAACATATTCGA | |
| | | | | CTTGCTGCTCGCGGCCGCCACCGCCGGGCCCCGGC | |
| | | | | CGTCCCTGGCTCCCCTCCTGCCTCGA | |
| HPV31 | bkpt1- | 939 | 940 | ATATTAGAACATTATGAAAATGATAGTAAACGAC | 1970 |
| | MYC_001_exon2 | | | TTTGTGATCATATAGACTATTGGAAACATATTCGA | |
| | | | | CTTGCAGCCTCCCGCGACGATGCCCCTCAACGTTA | |
| | | | | GCTTCACCAACAGGAACTATGACCTCGACTACG | |
| HPV31 | bkpt1- | 941 | 942 | ATATTAGAACATTATGAAAATGATAGTAAACGAC | 1971 |
| | MYC_001_exon3 | | | TTTGTGATCATATAGACTATTGGAAACATATTCGA | |
| | | | | CTTGAGGAGGAACAAGAAGATGAGGAAGAAATC | |
| | | | | GATGTTGTTTCTGTGGAAAAGAGGCAGGCTCCTGG | |
| HPV31 | bkpt1- | 943 | 944 | ATATTAGAACATTATGAAAATGATAGTAAACGAC | 1972 |
| | PVT1_002_exon3 | | | TTTGTGATCATATAGACTATTGGAAACATATTCGA | |
| | | | | CTTGCTGACCATACTCCCTGGAGCCTTCTCCCGAG | |
| | | | | GTGCGCGGGTGACCT | |
| HPV31 | bkpt1- | 945 | 946 | ATATTAGAACATTATGAAAATGATAGTAAACGAC | 1973 |
| | PVT1_004_exon1 | | | TTTGTGATCATATAGACTATTGGAAACATATTCGA | |
| | | | | CTTGTCTGAGCCTGATGGATTTACAGTGATCTTCA | |
| | | | | GTGGTCTGGGG | |
| HPV31 | bkpt1- | 947 | 948 | ATATTAGAACATTATGAAAATGATAGTAAACGAC | 1974 |
| | PVT1_005_exon1 | | | TTTGTGATCATATAGACTATTGGAAACATATTCGA | |
| | | | | CTTGCTCCGGGCAGAGCGCGTGTGGCGGCCGAGC | |
| | | | | ACATGGGCCCGCGGGCCGGGCGGGCTCGGGGCGG | |
| | | | | CCGGGACGAGGAGGGGCGACG | |
| HPV31 | bkpt2- | 949 | 950 | AACAAGGGCTGTCAGTTGTCCTGCAACTACACCTA | 1975 |
| | MYC_001_exon1 | | | TAACTGCTCGCGGCCGCCACCGCCGGGCCCCGGC | |
| | | | | CGTCCCTGGCTCCCCTCCTGCCTCGA | |
| HPV31 | bkpt2- | 951 | 952 | AACAAGGGCTGTCAGTTGTCCTGCAACTACACCTA | 1976 |
| | MYC_001_exon2 | | | TAACAGCCTCCCGCGACGATGCCCCTCAACGTTAG | |
| | | | | CTTCACCAACAGGAACTATGACCTCGACTACG | |
| HPV31 | bkpt2- | 953 | 954 | AACAAGGGCTGTCAGTTGTCCTGCAACTACACCTA | 1977 |
| | MYC_001_exon3 | | | TAAAGGAGGAACAAGAAGATGAGGAAGAAATCG | |
| | | | | ATGTTGTTTCTGTGGAAAAGAGGCAGGCTCCTGG | |
| HPV31 | bkpt2- | 955 | 956 | AACAAGGGCTGTCAGTTGTCCTGCAACTACACCTA | 1978 |
| | PVT1_002_exon3 | | | TAACTGACCATACTCCCTGGAGCCTTCTCCCGAGG | |
| | | | | TGCGCGGGTGACCT | |
| HPV31 | bkpt2- | 957 | 958 | AACAAGGGCTGTCAGTTGTCCTGCAACTACACCTA | 1979 |
| | PVT1_004_exon1 | | | TAATCTGAGCCTGATGGATTTACAGTGATCTTCAG | |
| | | | | TGGTCTGGGG | |
| HPV31 | bkpt2- | 959 | 960 | AACAAGGGCTGTCAGTTGTCCTGCAACTACACCTA | 1980 |
| | PVT1_005_exon1 | | | TAACTCCGGGCAGAGCGCGTGTGGCGGCCGAGCA | |
| | | | | CATGGGCCCGCGGGCCGGGCGGGCTCGGGGCGGC | |
| | | | | CGGGACGAGGAGGGGCGACG | |
| HPV31 | SD2- | 961 | 962 | TCTACTAGACTGTAACTACAATGGCTGATCCAGCA | 1981 |
| | MYC_001_exon1 | | | GCTGCTCGCGGCCGCCACCGCCGGGCCCCGGCCG | |
| | | | | TCCCTGGCTCCCCTCCTGCCTCGA | |
| HPV31 | SD2- | 963 | 964 | TCTACTAGACTGTAACTACAATGGCTGATCCAGCA | 1982 |
| | MYC_001_exon2 | | | GCAGCCTCCCGCGACGATGCCCCTCAACGTTAGCT | |
| | | | | TCACCAACAGGAACTATGACCTCGACTACG | |
| HPV31 | SD2- | 965 | 966 | TCTACTAGACTGTAACTACAATGGCTGATCCAGCA | 1983 |
| | MYC_001_exon3 | | | GAGGAGGAACAAGAAGATGAGGAAGAAATCGAT | |
| | | | | GTTGTTTCTGTGGAAAAGAGGCAGGCTCCTGG | |
| HPV31 | SD2- | 967 | 968 | TCTACTAGACTGTAACTACAATGGCTGATCCAGCA | 1984 |
| | PVT1_002_exon3 | | | GCTGACCATACTCCCTGGAGCCTTCTCCCGAGGTG | |
| | | | | CGCGGGTGACCT | |
| HPV31 | SD2- | 969 | 970 | TCTACTAGACTGTAACTACAATGGCTGATCCAGCA | 1985 |
| | PVT1_004_exon1 | | | GTCTGAGCCTGATGGATTTACAGTGATCTTCAGTG | |
| | | | | GTCTGGGG | |
| HPV31 | SD2- | 971 | 972 | TCTACTAGACTGTAACTACAATGGCTGATCCAGCA | 1986 |
| | PVT1_005_exon1 | | | GCTCCGGGCAGAGCGCGTGTGGCGGCCGAGCACA | |
| | | | | TGGGCCCGCGGGCCGGGCGGGCTCGGGGCGGCCG | |
| | | | | GGACGAGGAGGGGCGACG | |
| HPV33 | bkpt1- | 973 | 974 | AGCTGATAAAACTGATTTACCATCACAAATTGAAC | 1987 |
| | MYC_001_exon1 | | | ATTGGAAACTGATACGCATGGCTGCTCGCGGCCG | |
| | | | | CCACCGCCGGGCCCCGGCCGTCCCTGGCTCCCCTC | |
| | | | | CTGCCTCGA | |
| HPV33 | bkpt1- | 975 | 976 | AGCTGATAAAACTGATTTACCATCACAAATTGAAC | 1988 |
| | MYC_001_exon2 | | | ATTGGAAACTGATACGCATGGCAGCCTCCCGCGA | |
| | | | | CGATGCCCCTCAACGTTAGCTTCACCAACAGGAAC | |
| | | | | TATGACCTCGACTACG | |
| HPV33 | bkpt1- | 977 | 978 | AGCTGATAAAACTGATTTACCATCACAAATTGAAC | 1989 |
| | MYC_001_exon3 | | | ATTGGAAACTGATACGCATGGAGGAGGAACAAGA | |
| | | | | AGATGAGGAAGAAATCGATGTTGTTTCTGTGGAA | |
| | | | | AAGAGGCAGGCTCCTGG | |
| HPV33 | bkpt1- | 979 | 980 | AGCTGATAAAACTGATTTACCATCACAAATTGAAC | 1990 |
| | PVT1_002_exon3 | | | ATTGGAAACTGATACGCATGGCTGACCATACTCCC | |
| | | | | TGGAGCCTTCTCCCGAGGTGCGCGGGTGACCT | |
| HPV33 | bkpt1- | 981 | 982 | AGCTGATAAAACTGATTTACCATCACAAATTGAAC | 1991 |
| | PVT1_004_exon1 | | | ATTGGAAACTGATACGCATGGTCTGAGCCTGATG | |
| | | | | GATTTACAGTGATCTTCAGTGGTCTGGGG | |
| HPV33 | bkpt1- | 983 | 984 | AGCTGATAAAACTGATTTACCATCACAAATTGAAC | 1992 |
| | PVT1_005_exon1 | | | ATTGGAAACTGATACGCATGGCTCCGGGCAGAGC | |
| | | | | GCGTGTGGCGGCCGAGCACATGGGCCCGCGGGCC | |
| | | | | GGGCGGGCTCGGGGCGGCCGGGACGAGGAGGGG | |
| | | | | CGACG | |
| HPV33 | bkpt2- | 985 | 986 | ACAAGCAGCGGACTGTGTGTAGTTCTAACGTTGCA | 1993 |
| | MYC_001_exon1 | | | CCTATAGCTGCTCGCGGCCGCCACCGCCGGGCCCC | |
| | | | | GGCCGTCCCTGGCTCCCCTCCTGCCTCGA | |
| HPV33 | bkpt2- | 987 | 988 | ACAAGCAGCGGACTGTGTGTAGTTCTAACGTTGCA | 1994 |
| | MYC_001_exon2 | | | CCTATAGCAGCCTCCCGCGACGATGCCCCTCAACG | |
| | | | | TTAGCTTCACCAACAGGAACTATGACCTCGACTAC | |
| | | | | G | |
| HPV33 | bkpt2- | 989 | 990 | ACAAGCAGCGGACTGTGTGTAGTTCTAACGTTGCA | 1995 |
| | MYC_001_exon3 | | | CCTATAGAGGAGGAACAAGAAGATGAGGAAGAA | |
| | | | | ATCGATGTTGTTTCTGTGGAAAAGAGGCAGGCTCC | |
| | | | | TGG | |
| HPV33 | bkpt2- | 991 | 992 | ACAAGCAGCGGACTGTGTGTAGTTCTAACGTTGCA | 1996 |
| | PVT1_002_exon3 | | | CCTATAGCTGACCATACTCCCTGGAGCCTTCTCCC | |
| | | | | GAGGTGCGCGGGTGACCT | |
| HPV33 | bkpt2- | 993 | 994 | ACAAGCAGCGGACTGTGTGTAGTTCTAACGTTGCA | 1997 |
| | PVT1_004_exon1 | | | CCTATAGTCTGAGCCTGATGGATTTACAGTGATCT | |
| | | | | TCAGTGGTCTGGGG | |
| HPV33 | bkpt2- | 995 | 996 | ACAAGCAGCGGACTGTGTGTAGTTCTAACGTTGCA | 1998 |
| | PVT1_005_exon1 | | | CCTATAGCTCCGGGCAGAGCGCGTGTGGCGGCCG | |
| | | | | AGCACATGGGCCCGCGGGCCGGGCGGGCTCGGGG | |
| | | | | CGGCCGGGACGAGGAGGGGCGACG | |
| HPV33 | SD2- | 997 | 998 | CAATAAACATCATCTACAATGGCCGATCCTGAAG | 1999 |
| | MYC_001_exon1 | | | CTGCTCGCGGCCGCCACCGCCGGGCCCCGGCCGTC | |
| | | | | CCTGGCTCCCCTCCTGCCTCGA | |
| HPV33 | SD2- | 999 | 1000 | CAATAAACATCATCTACAATGGCCGATCCTGAAG | 2000 |
| | MYC_001_exon2 | | | CAGCCTCCCGCGACGATGCCCCTCAACGTTAGCTT | |
| | | | | CACCAACAGGAACTATGACCTCGACTACG | |
| HPV33 | SD2- | 1001 | 1002 | CAATAAACATCATCTACAATGGCCGATCCTGAAG | 2001 |
| | MYC_001_exon3 | | | AGGAGGAACAAGAAGATGAGGAAGAAATCGATG | |
| | | | | TTGTTTCTGTGGAAAAGAGGCAGGCTCCTGG | |
| HPV33 | SD2- | 1003 | 1004 | CAATAAACATCATCTACAATGGCCGATCCTGAAG | 2002 |
| | PVT1_002_exon3 | | | CTGACCATACTCCCTGGAGCCTTCTCCCGAGGTGC | |
| | | | | GCGGGTGACCT | |
| HPV33 | SD2- | 1005 | 1006 | CAATAAACATCATCTACAATGGCCGATCCTGAAGT | 2003 |
| | PVT1_004_exon1 | | | CTGAGCCTGATGGATTTACAGTGATCTTCAGTGGT | |
| | | | | CTGGGG | |
| HPV33 | SD2- | 1007 | 1008 | CAATAAACATCATCTACAATGGCCGATCCTGAAG | 2004 |
| | PVT1_005_exon1 | | | CTCCGGGCAGAGCGCGTGTGGCGGCCGAGCACAT | |
| | | | | GGGCCCGCGGGCCGGGCGGGCTCGGGGCGGCCGG | |
| | | | | GACGAGGAGGGGCGACG | |
| HPV35 | bkpt1- | 1009 | 1010 | CTGATCACATACAGTATTGGAAACTGATTCGTCTT | 2005 |
| | MYC_001_exon1 | | | GCTGCTCGCGGCCGCCACCGCCGGGCCCCGGCCG | |
| | | | | TCCCTGGCTCCCCTCCTGCCTCGA | |
| HPV35 | bkpt1- | 1011 | 1012 | CTGATCACATACAGTATTGGAAACTGATTCGTCTT | 2006 |
| | MYC_001_exon2 | | | GCAGCCTCCCGCGACGATGCCCCTCAACGTTAGCT | |
| | | | | TCACCAACAGGAACTATGACCTCGACTACG | |
| HPV35 | bkpt1- | 1013 | 1014 | CTGATCACATACAGTATTGGAAACTGATTCGTCTT | 2007 |
| | MYC_001_exon3 | | | GAGGAGGAACAAGAAGATGAGGAAGAAATCGAT | |
| | | | | GTTGTTTCTGTGGAAAAGAGGCAGGCTCCTGG | |
| HPV35 | bkpt1- | 1015 | 1016 | CTGATCACATACAGTATTGGAAACTGATTCGTCTT | 2008 |
| | PVT1_002_exon3 | | | GCTGACCATACTCCCTGGAGCCTTCTCCCGAGGTG | |
| | | | | CGCGGGTGACCT | |
| HPV35 | bkpt1- | 1017 | 1018 | CTGATCACATACAGTATTGGAAACTGATTCGTCTT | 2009 |
| | PVT1_004_exon1 | | | GTCTGAGCCTGATGGATTTACAGTGATCTTCAGTG | |
| | | | | GTCTGGGG | |
| HPV35 | bkpt1- | 1019 | 1020 | CTGATCACATACAGTATTGGAAACTGATTCGTCTT | 2010 |
| | PVT1_005_exon1 | | | GCTCCGGGCAGAGCGCGTGTGGCGGCCGAGCACA | |
| | | | | TGGGCCCGCGGGCCGGGCGGGCTCGGGGCGGCCG | |
| | | | | GGACGAGGAGGGGCGACG | |
| HPV35 | bkpt2- | 1021 | 1022 | CCGGTGTGGTAGTTGTAGTACAACTACACCTATAG | 2011 |
| | MYC_001_exon1 | | | CTGCTCGCGGCCGCCACCGCCGGGCCCCGGCCGTC | |
| | | | | CCTGGCTCCCCTCCTGCCTCGA | |
| HPV35 | bkpt2- | 1023 | 1024 | CCGGTGTGGTAGTTGTAGTACAACTACACCTATAG | 2012 |
| | MYC_001_exon2 | | | CAGCCTCCCGCGACGATGCCCCTCAACGTTAGCTT | |
| | | | | CACCAACAGGAACTATGACCTCGACTACG | |
| HPV35 | bkpt2- | 1025 | 1026 | CCGGTGTGGTAGTTGTAGTACAACTACACCTATAG | 2013 |
| | MYC_001_exon3 | | | AGGAGGAACAAGAAGATGAGGAAGAAATCGATG | |
| | | | | TTGTTTCTGTGGAAAAGAGGCAGGCTCCTGG | |
| HPV35 | bkpt2- | 1027 | 1028 | CCGGTGTGGTAGTTGTAGTACAACTACACCTATAG | 2014 |
| | PVT1_002_exon3 | | | CTGACCATACTCCCTGGAGCCTTCTCCCGAGGTGC | |
| | | | | GCGGGTGACCT | |
| HPV35 | bkpt2- | 1029 | 1030 | CCGGTGTGGTAGTTGTAGTACAACTACACCTATAG | 2015 |
| | PVT1_004_exon1 | | | TCTGAGCCTGATGGATTTACAGTGATCTTCAGTGG | |
| | | | | TCTGGGG | |
| HPV35 | bkpt2- | 1031 | 1032 | CCGGTGTGGTAGTTGTAGTACAACTACACCTATAG | 2016 |
| | PVT1_005_exon1 | | | CTCCGGGCAGAGCGCGTGTGGCGGCCGAGCACAT | |
| | | | | GGGCCCGCGGGCCGGGCGGGCTCGGGGCGGCCGG | |
| | | | | GACGAGGAGGGGCGACG | |
| HPV35 | SD2- | 1033 | 1034 | CTACAATGGCTGATCCTGCAGCTGCTCGCGGCCGC | 2017 |
| | MYC_001_exon1 | | | CACCGCCGGGCCCCGGCCGTCCCTGGCTCCCCTCC | |
| | | | | TGCCTCGA | |
| HPV35 | SD2- | 1035 | 1036 | CTACAATGGCTGATCCTGCAGCAGCCTCCCGCGAC | 2018 |
| | MYC_001_exon2 | | | GATGCCCCTCAACGTTAGCTTCACCAACAGGAACT | |
| | | | | ATGACCTCGACTACG | |
| HPV35 | SD2- | 1037 | 1038 | CTACAATGGCTGATCCTGCAGAGGAGGAACAAGA | 2019 |
| | MYC_001_exon3 | | | AGATGAGGAAGAAATCGATGTTGTTTCTGTGGAA | |
| | | | | AAGAGGCAGGCTCCTGG | |
| HPV35 | SD2- | 1039 | 1040 | CTACAATGGCTGATCCTGCAGCTGACCATACTCCC | 2020 |
| | PVT1_002_exon3 | | | TGGAGCCTTCTCCCGAGGTGCGCGGGTGACCT | |
| HPV35 | SD2- | 1041 | 1042 | CTACAATGGCTGATCCTGCAGTCTGAGCCTGATGG | 2021 |
| | PVT1_004_exon1 | | | ATTTACAGTGATCTTCAGTGGTCTGGGG | |
| HPV35 | SD2- | 1043 | 1044 | CTACAATGGCTGATCCTGCAGCTCCGGGCAGAGC | 2022 |
| | PVT1_005_exon1 | | | GCGTGTGGCGGCCGAGCACATGGGCCCGCGGGCC | |
| | | | | GGGCGGGCTCGGGGCGGCCGGGACGAGGAGGGG | |
| | | | | CGACG | |
| HPV39 | bkpt1- | 1045 | 1046 | AAATACTAGAATACTATGAACAAGACAGTAAATC | 2023 |
| | MYC_001_exon1 | | | AATATATGATCAAATTAATTATTGGAAATGTGTGC | |
| | | | | GAATGGCTGCTCGCGGCCGCCACCGCCGGGCCCC | |
| | | | | GGCCGTCCCTGGCTCCCCTCCTGCCTCGA | |
| HPV39 | bkpt1- | 1047 | 1048 | AAATACTAGAATACTATGAACAAGACAGTAAATC | 2024 |
| | MYC_001_exon2 | | | AATATATGATCAAATTAATTATTGGAAATGTGTGC | |
| | | | | GAATGGCAGCCTCCCGCGACGATGCCCCTCAACG | |
| | | | | TTAGCTTCACCAACAGGAACTATGACCTCGACTAC | |
| | | | | G | |
| HPV39 | bkpt1- | 1049 | 1050 | AAATACTAGAATACTATGAACAAGACAGTAAATC | 2025 |
| | MYC_001_exon3 | | | AATATATGATCAAATTAATTATTGGAAATGTGTGC | |
| | | | | GAATGGAGGAGGAACAAGAAGATGAGGAAGAAA | |
| | | | | TCGATGTTGTTTCTGTGGAAAAGAGGCAGGCTCCT | |
| | | | | GG | |
| HPV39 | bkpt1- | 1051 | 1052 | AAATACTAGAATACTATGAACAAGACAGTAAATC | 2026 |
| | PVT1_002_exon3 | | | AATATATGATCAAATTAATTATTGGAAATGTGTGC | |
| | | | | GAATGGCTGACCATACTCCCTGGAGCCTTCTCCCG | |
| | | | | AGGTGCGCGGGTGACCT | |
| HPV39 | bkpt1- | 1053 | 1054 | AAATACTAGAATACTATGAACAAGACAGTAAATC | 2027 |
| | PVT1_004_exon1 | | | AATATATGATCAAATTAATTATTGGAAATGTGTGC | |
| | | | | GAATGGTCTGAGCCTGATGGATTTACAGTGATCTT | |
| | | | | CAGTGGTCTGGGG | |
| HPV39 | bkpt1- | 1055 | 1056 | AAATACTAGAATACTATGAACAAGACAGTAAATC | 2028 |
| | PVT1_005_exon1 | | | AATATATGATCAAATTAATTATTGGAAATGTGTGC | |
| | | | | GAATGGCTCCGGGCAGAGCGCGTGTGGCGGCCGA | |
| | | | | GCACATGGGCCCGCGGGCCGGGCGGGCTCGGGGC | |
| | | | | GGCCGGGACGAGGAGGGGCGACG | |
| HPV39 | bkpt2- | 1057 | 1058 | ACACAAGACGGTACCTCAGTTGTGGTAACACTAC | 2029 |
| | MYC_001_exon1 | | | GCCTATAACTGCTCGCGGCCGCCACCGCCGGGCCC | |
| | | | | CGGCCGTCCCTGGCTCCCCTCCTGCCTCGA | |
| HPV39 | bkpt2- | 1059 | 1060 | ACACAAGACGGTACCTCAGTTGTGGTAACACTAC | 2030 |
| | MYC_001_exon2 | | | GCCTATAACAGCCTCCCGCGACGATGCCCCTCAAC | |
| | | | | GTTAGCTTCACCAACAGGAACTATGACCTCGACTA | |
| | | | | CG | |
| HPV39 | bkpt2- | 1061 | 1062 | ACACAAGACGGTACCTCAGTTGTGGTAACACTAC | 2031 |
| | MYC_001_exon3 | | | GCCTATAAAGGAGGAACAAGAAGATGAGGAAGA | |
| | | | | AATCGATGTTGTTTCTGTGGAAAAGAGGCAGGCTC | |
| | | | | CTGG | |
| HPV39 | bkpt2- | 1063 | 1064 | ACACAAGACGGTACCTCAGTTGTGGTAACACTAC | 2032 |
| | PVT1_002_exon3 | | | GCCTATAACTGACCATACTCCCTGGAGCCTTCTCC | |
| | | | | CGAGGTGCGCGGGTGACCT | |
| HPV39 | bkpt2- | 1065 | 1066 | ACACAAGACGGTACCTCAGTTGTGGTAACACTAC | 2033 |
| | PVT1_004_exon1 | | | GCCTATAATCTGAGCCTGATGGATTTACAGTGATC | |
| | | | | TTCAGTGGTCTGGGG | |
| HPV39 | bkpt2- | 1067 | 1068 | ACACAAGACGGTACCTCAGTTGTGGTAACACTAC | 2034 |
| | PVT1_005_exon1 | | | GCCTATAACTCCGGGCAGAGCGCGTGTGGCGGCC | |
| | | | | GAGCACATGGGCCCGCGGGCCGGGCGGGCTCGGG | |
| | | | | GCGGCCGGGACGAGGAGGGGCGACG | |
| HPV39 | SD2- | 1069 | 1070 | ACCAGTAACCTGCTATGGCCAATCGTGAAGCTGCT | 2035 |
| | MYC_001_exon1 | | | CGCGGCCGCCACCGCCGGGCCCCGGCCGTCCCTG | |
| | | | | GCTCCCCTCCTGCCTCGA | |
| HPV39 | SD2- | 1071 | 1072 | ACCAGTAACCTGCTATGGCCAATCGTGAAGCAGC | 2036 |
| | MYC_001_exon2 | | | CTCCCGCGACGATGCCCCTCAACGTTAGCTTCACC | |
| | | | | AACAGGAACTATGACCTCGACTACG | |
| HPV39 | SD2- | 1073 | 1074 | ACCAGTAACCTGCTATGGCCAATCGTGAAGAGGA | 2037 |
| | MYC_001_exon3 | | | GGAACAAGAAGATGAGGAAGAAATCGATGTTGTT | |
| | | | | TCTGTGGAAAAGAGGCAGGCTCCTGG | |
| HPV39 | SD2- | 1075 | 1076 | ACCAGTAACCTGCTATGGCCAATCGTGAAGCTGA | 2038 |
| | PVT1_002_exon3 | | | CCATACTCCCTGGAGCCTTCTCCCGAGGTGCGCGG | |
| | | | | GTGACCT | |
| HPV39 | SD2- | 1077 | 1078 | ACCAGTAACCTGCTATGGCCAATCGTGAAGTCTGA | 2039 |
| | PVT1_004_exon1 | | | GCCTGATGGATTTACAGTGATCTTCAGTGGTCTGG | |
| | | | | GG | |
| HPV39 | SD2- | 1079 | 1080 | ACCAGTAACCTGCTATGGCCAATCGTGAAGCTCCG | 2040 |
| | PVT1_005_exon1 | | | GGCAGAGCGCGTGTGGCGGCCGAGCACATGGGCC | |
| | | | | CGCGGGCCGGGCGGGCTCGGGGCGGCCGGGACGA | |
| | | | | GGAGGGGCGACG | |
| HPV45 | bkpt1- | 1081 | 1082 | TGAAAATGACAGTAAAGACATAAACAGCCAAATA | 2041 |
| | MYC_001_exon1 | | | AGTTATTGGCAACTTATACGTTTGGCTGCTCGCGG | |
| | | | | CCGCCACCGCCGGGCCCCGGCCGTCCCTGGCTCCC | |
| | | | | CTCCTGCCTCGA | |
| HPV45 | bkpt1- | 1083 | 1084 | TGAAAATGACAGTAAAGACATAAACAGCCAAATA | 2042 |
| | MYC_001_exon2 | | | AGTTATTGGCAACTTATACGTTTGGCAGCCTCCCG | |
| | | | | CGACGATGCCCCTCAACGTTAGCTTCACCAACAGG | |
| | | | | AACTATGACCTCGACTACG | |
| HPV45 | bkpt1- | 1085 | 1086 | TGAAAATGACAGTAAAGACATAAACAGCCAAATA | 2043 |
| | MYC_001_exon3 | | | AGTTATTGGCAACTTATACGTTTGGAGGAGGAAC | |
| | | | | AAGAAGATGAGGAAGAAATCGATGTTGTTTCTGT | |
| | | | | GGAAAAGAGGCAGGCTCCTGG | |
| HPV45 | bkpt1- | 1087 | 1088 | TGAAAATGACAGTAAAGACATAAACAGCCAAATA | 2044 |
| | PVT1_002_exon3 | | | AGTTATTGGCAACTTATACGTTTGGCTGACCATAC | |
| | | | | TCCCTGGAGCCTTCTCCCGAGGTGCGCGGGTGACC | |
| | | | | T | |
| HPV45 | bkpt1- | 1089 | 1090 | TGAAAATGACAGTAAAGACATAAACAGCCAAATA | 2045 |
| | PVT1_004_exon1 | | | AGTTATTGGCAACTTATACGTTTGGTCTGAGCCTG | |
| | | | | ATGGATTTACAGTGATCTTCAGTGGTCTGGGG | |
| HPV45 | bkpt1- | 1091 | 1092 | TGAAAATGACAGTAAAGACATAAACAGCCAAATA | 2046 |
| | PVT1_005_exon1 | | | AGTTATTGGCAACTTATACGTTTGGCTCCGGGCAG | |
| | | | | AGCGCGTGTGGCGGCCGAGCACATGGGCCCGCGG | |
| | | | | GCCGGGCGGGCTCGGGGCGGCCGGGACGAGGAG | |
| | | | | GGGCGACG | |
| HPV45 | bkpt2- | 1093 | 1094 | AAGAAGGAAAGTGTGTAGTGGTAACACTACGCCT | 2047 |
| | MYC_001_exon1 | | | ATAACTGCTCGCGGCCGCCACCGCCGGGCCCCGG | |
| | | | | CCGTCCCTGGCTCCCCTCCTGCCTCGA | |
| HPV45 | bkpt2- | 1095 | 1096 | AAGAAGGAAAGTGTGTAGTGGTAACACTACGCCT | 2048 |
| | MYC_001_exon2 | | | ATAACAGCCTCCCGCGACGATGCCCCTCAACGTTA | |
| | | | | GCTTCACCAACAGGAACTATGACCTCGACTACG | |
| HPV45 | bkpt2- | 1097 | 1098 | AAGAAGGAAAGTGTGTAGTGGTAACACTACGCCT | 2049 |
| | MYC_001_exon3 | | | ATAAAGGAGGAACAAGAAGATGAGGAAGAAATC | |
| | | | | GATGTTGTTTCTGTGGAAAAGAGGCAGGCTCCTGG | |
| HPV45 | bkpt2- | 1099 | 1100 | AAGAAGGAAAGTGTGTAGTGGTAACACTACGCCT | 2050 |
| | PVT1_002_exon3 | | | ATAACTGACCATACTCCCTGGAGCCTTCTCCCGAG | |
| | | | | GTGCGCGGGTGACCT | |
| HPV45 | bkpt2- | 1101 | 1102 | AAGAAGGAAAGTGTGTAGTGGTAACACTACGCCT | 2051 |
| | PVT1_004_exon1 | | | ATAATCTGAGCCTGATGGATTTACAGTGATCTTCA | |
| | | | | GTGGTCTGGGG | |
| HPV45 | bkpt2- | 1103 | 1104 | AAGAAGGAAAGTGTGTAGTGGTAACACTACGCCT | 2052 |
| | PVT1_005_exon1 | | | ATAACTCCGGGCAGAGCGCGTGTGGCGGCCGAGC | |
| | | | | ACATGGGCCCGCGGGCCGGGCGGGCTCGGGGCGG | |
| | | | | CCGGGACGAGGAGGGGCGACG | |
| HPV45 | SD2- | 1105 | 1106 | CCGTGGTGTGCAACTAACCAATAATCTACAATGGC | 2053 |
| | MYC_001_exon1 | | | GGATCCAGAAGCTGCTCGCGGCCGCCACCGCCGG | |
| | | | | GCCCCGGCCGTCCCTGGCTCCCCTCCTGCCTCGA | |
| HPV45 | SD2- | 1107 | 1108 | CCGTGGTGTGCAACTAACCAATAATCTACAATGGC | 2054 |
| | MYC_001_exon2 | | | GGATCCAGAAGCAGCCTCCCGCGACGATGCCCCT | |
| | | | | CAACGTTAGCTTCACCAACAGGAACTATGACCTCG | |
| | | | | ACTACG | |
| HPV45 | SD2- | 1109 | 1110 | CCGTGGTGTGCAACTAACCAATAATCTACAATGGC | 2055 |
| | MYC_001_exon3 | | | GGATCCAGAAGAGGAGGAACAAGAAGATGAGGA | |
| | | | | AGAAATCGATGTTGTTTCTGTGGAAAAGAGGCAG | |
| | | | | GCTCCTGG | |
| HPV45 | SD2- | 1111 | 1112 | CCGTGGTGTGCAACTAACCAATAATCTACAATGGC | 2056 |
| | PVT1_002_exon3 | | | GGATCCAGAAGCTGACCATACTCCCTGGAGCCTTC | |
| | | | | TCCCGAGGTGCGCGGGTGACCT | |
| HPV45 | SD2- | 1113 | 1114 | CCGTGGTGTGCAACTAACCAATAATCTACAATGGC | 2057 |
| | PVT1_004_exon1 | | | GGATCCAGAAGTCTGAGCCTGATGGATTTACAGT | |
| | | | | GATCTTCAGTGGTCTGGGG | |
| HPV45 | SD2- | 1115 | 1116 | CCGTGGTGTGCAACTAACCAATAATCTACAATGGC | 2058 |
| | PVT1_005_exon1 | | | GGATCCAGAAGCTCCGGGCAGAGCGCGTGTGGCG | |
| | | | | GCCGAGCACATGGGCCCGCGGGCCGGGCGGGCTC | |
| | | | | GGGGCGGCCGGGACGAGGAGGGGCGACG | |
| HPV51 | bkpt1- | 1117 | 1118 | GAACTGGACAGTGATAAATTAGTAGATCAAATTA | 2059 |
| | MYC_001_exon1 | | | ACTATTGGACATTGTTACGATATGCTGCTCGCGGC | |
| | | | | CGCCACCGCCGGGCCCCGGCCGTCCCTGGCTCCCC | |
| | | | | TCCTGCCTCGA | |
| HPV51 | bkpt1- | 1119 | 1120 | GAACTGGACAGTGATAAATTAGTAGATCAAATTA | 2060 |
| | MYC_001_exon2 | | | ACTATTGGACATTGTTACGATATGCAGCCTCCCGC | |
| | | | | GACGATGCCCCTCAACGTTAGCTTCACCAACAGG | |
| | | | | AACTATGACCTCGACTACG | |
| HPV51 | bkpt1- | 1121 | 1122 | GAACTGGACAGTGATAAATTAGTAGATCAAATTA | 2061 |
| | MYC_001_exon3 | | | ACTATTGGACATTGTTACGATATGAGGAGGAACA | |
| | | | | AGAAGATGAGGAAGAAATCGATGTTGTTTCTGTG | |
| | | | | GAAAAGAGGCAGGCTCCTGG | |
| HPV51 | bkpt1- | 1123 | 1124 | GAACTGGACAGTGATAAATTAGTAGATCAAATTA | 2062 |
| | PVT1_002_exon3 | | | ACTATTGGACATTGTTACGATATGCTGACCATACT | |
| | | | | CCCTGGAGCCTTCTCCCGAGGTGCGCGGGTGACCT | |
| HPV51 | bkpt1- | 1125 | 1126 | GAACTGGACAGTGATAAATTAGTAGATCAAATTA | 2063 |
| | PVT1_004_exon1 | | | ACTATTGGACATTGTTACGATATGTCTGAGCCTGA | |
| | | | | TGGATTTACAGTGATCTTCAGTGGTCTGGGG | |
| HPV51 | bkpt1- | 1127 | 1128 | GAACTGGACAGTGATAAATTAGTAGATCAAATTA | 2064 |
| | PVT1_005_exon1 | | | ACTATTGGACATTGTTACGATATGCTCCGGGCAGA | |
| | | | | GCGCGTGTGGCGGCCGAGCACATGGGCCCGCGGG | |
| | | | | CCGGGCGGGCTCGGGGCGGCCGGGACGAGGAGG | |
| | | | | GGCGACG | |
| HPV51 | bkpt2- | 1129 | 1130 | GTGCAACTCAGACTGCGTTTATAGCTGCTCGCGGC | 2065 |
| | MYC_001_exon1 | | | CGCCACCGCCGGGCCCCGGCCGTCCCTGGCTCCCC | |
| | | | | TCCTGCCTCGA | |
| HPV51 | bkpt2- | 1131 | 1132 | GTGCAACTCAGACTGCGTTTATAGCAGCCTCCCGC | 2066 |
| | MYC_001_exon2 | | | GACGATGCCCCTCAACGTTAGCTTCACCAACAGG | |
| | | | | AACTATGACCTCGACTACG | |
| HPV51 | bkpt2- | 1133 | 1134 | GTGCAACTCAGACTGCGTTTATAGAGGAGGAACA | 2067 |
| | MYC_001_exon3 | | | AGAAGATGAGGAAGAAATCGATGTTGTTTCTGTG | |
| | | | | GAAAAGAGGCAGGCTCCTGG | |
| HPV51 | bkpt2- | 1135 | 1136 | GTGCAACTCAGACTGCGTTTATAGCTGACCATACT | 2068 |
| | PVT1_002_exon3 | | | CCCTGGAGCCTTCTCCCGAGGTGCGCGGGTGACCT | |
| HPV51 | bkpt2- | 1137 | 1138 | GTGCAACTCAGACTGCGTTTATAGTCTGAGCCTGA | 2069 |
| | PVT1_004_exon1 | | | TGGATTTACAGTGATCTTCAGTGGTCTGGGG | |
| HPV51 | bkpt2- | 1139 | 1140 | GTGCAACTCAGACTGCGTTTATAGCTCCGGGCAGA | 2070 |
| | PVT1_005_exon1 | | | GCGCGTGTGGCGGCCGAGCACATGGGCCCGCGGG | |
| | | | | CCGGGCGGGCTCGGGGCGGCCGGGACGAGGAGG | |
| | | | | GGCGACG | |
| HPV51 | SD2- | 1141 | 1142 | GCCCGTGTTGTGCGAACAACTAGCAACGGCGATG | 2071 |
| | MYC_001_exon1 | | | GACTGTGAAGCTGCTCGCGGCCGCCACCGCCGGG | |
| | | | | CCCCGGCCGTCCCTGGCTCCCCTCCTGCCTCGA | |
| HPV51 | SD2- | 1143 | 1144 | GCCCGTGTTGTGCGAACAACTAGCAACGGCGATG | 2072 |
| | MYC_001_exon2 | | | GACTGTGAAGCAGCCTCCCGCGACGATGCCCCTC | |
| | | | | AACGTTAGCTTCACCAACAGGAACTATGACCTCG | |
| | | | | ACTACG | |
| HPV51 | SD2- | 1145 | 1146 | GCCCGTGTTGTGCGAACAACTAGCAACGGCGATG | 2073 |
| | MYC_001_exon3 | | | GACTGTGAAGAGGAGGAACAAGAAGATGAGGAA | |
| | | | | GAAATCGATGTTGTTTCTGTGGAAAAGAGGCAGG | |
| | | | | CTCCTGG | |
| HPV51 | SD2- | 1147 | 1148 | GCCCGTGTTGTGCGAACAACTAGCAACGGCGATG | 2074 |
| | PVT1_002_exon3 | | | GACTGTGAAGCTGACCATACTCCCTGGAGCCTTCT | |
| | | | | CCCGAGGTGCGCGGGTGACCT | |
| HPV51 | SD2- | 1149 | 1150 | GCCCGTGTTGTGCGAACAACTAGCAACGGCGATG | 2075 |
| | PVT1_004_exon1 | | | GACTGTGAAGTCTGAGCCTGATGGATTTACAGTGA | |
| | | | | TCTTCAGTGGTCTGGGG | |
| HPV51 | SD2- | 1151 | 1152 | GCCCGTGTTGTGCGAACAACTAGCAACGGCGATG | 2076 |
| | PVT1_005_exon1 | | | GACTGTGAAGCTCCGGGCAGAGCGCGTGTGGCGG | |
| | | | | CCGAGCACATGGGCCCGCGGGCCGGGCGGGCTCG | |
| | | | | GGGCGGCCGGGACGAGGAGGGGCGACG | |
| HPV52 | bkpt1- | 1153 | 1154 | TTGAACATTGGAAATTGACTCGAATGGCTGCTCGC | 2077 |
| | MYC_001_exon1 | | | GGCCGCCACCGCCGGGCCCCGGCCGTCCCTGGCTC | |
| | | | | CCCTCCTGCCTCGA | |
| HPV52 | bkpt1- | 1155 | 1156 | TTGAACATTGGAAATTGACTCGAATGGCAGCCTCC | 2078 |
| | MYC_001_exon2 | | | CGCGACGATGCCCCTCAACGTTAGCTTCACCAACA | |
| | | | | GGAACTATGACCTCGACTACG | |
| HPV52 | bkpt1- | 1157 | 1158 | TTGAACATTGGAAATTGACTCGAATGGAGGAGGA | 2079 |
| | MYC_001_exon3 | | | ACAAGAAGATGAGGAAGAAATCGATGTTGTTTCT | |
| | | | | GTGGAAAAGAGGCAGGCTCCTGG | |
| HPV52 | bkpt1- | 1159 | 1160 | TTGAACATTGGAAATTGACTCGAATGGCTGACCAT | 2080 |
| | PVT1_002_exon3 | | | ACTCCCTGGAGCCTTCTCCCGAGGTGCGCGGGTGA | |
| | | | | CCT | |
| HPV52 | bkpt1- | 1161 | 1162 | TTGAACATTGGAAATTGACTCGAATGGTCTGAGCC | 2081 |
| | PVT1_004_exon1 | | | TGATGGATTTACAGTGATCTTCAGTGGTCTGGGG | |
| HPV52 | bkpt1- | 1163 | 1164 | TTGAACATTGGAAATTGACTCGAATGGCTCCGGGC | 2082 |
| | PVT1_005_exon1 | | | AGAGCGCGTGTGGCGGCCGAGCACATGGGCCCGC | |
| | | | | GGGCCGGGCGGGCTCGGGGCGGCCGGGACGAGG | |
| | | | | AGGGGCGACG | |
| HPV52 | bkpt2- | 1165 | 1166 | ACAAAGGACGGGTTGCACATACAACTTGTACTGC | 2083 |
| | MYC_001_exon1 | | | ACCTATAACTGCTCGCGGCCGCCACCGCCGGGCCC | |
| | | | | CGGCCGTCCCTGGCTCCCCTCCTGCCTCGA | |
| HPV52 | bkpt2- | 1167 | 1168 | ACAAAGGACGGGTTGCACATACAACTTGTACTGC | 2084 |
| | MYC_001_exon2 | | | ACCTATAACAGCCTCCCGCGACGATGCCCCTCAAC | |
| | | | | GTTAGCTTCACCAACAGGAACTATGACCTCGACTA | |
| | | | | CG | |
| HPV52 | bkpt2- | 1169 | 1170 | ACAAAGGACGGGTTGCACATACAACTTGTACTGC | 2085 |
| | MYC_001_exon3 | | | ACCTATAAAGGAGGAACAAGAAGATGAGGAAGA | |
| | | | | AATCGATGTTGTTTCTGTGGAAAAGAGGCAGGCTC | |
| | | | | CTGG | |
| HPV52 | bkpt2- | 1171 | 1172 | ACAAAGGACGGGTTGCACATACAACTTGTACTGC | 2086 |
| | PVT1_002_exon3 | | | ACCTATAACTGACCATACTCCCTGGAGCCTTCTCC | |
| | | | | CGAGGTGCGCGGGTGACCT | |
| HPV52 | bkpt2- | 1173 | 1174 | ACAAAGGACGGGTTGCACATACAACTTGTACTGC | 2087 |
| | PVT1_004_exon1 | | | ACCTATAATCTGAGCCTGATGGATTTACAGTGATC | |
| | | | | TTCAGTGGTCTGGGG | |
| HPV52 | bkpt2- | 1175 | 1176 | ACAAAGGACGGGTTGCACATACAACTTGTACTGC | 2088 |
| | PVT1_005_exon1 | | | ACCTATAACTCCGGGCAGAGCGCGTGTGGCGGCC | |
| | | | | GAGCACATGGGCCCGCGGGCCGGGCGGGCTCGGG | |
| | | | | GCGGCCGGGACGAGGAGGGGCGACG | |
| HPV52 | SD2- | 1177 | 1178 | GTGTGCCCCGGCTGTGCACGGCTATAAACAACCCT | 2089 |
| | MYC_001_exon1 | | | GCAATGGAGGACCCTGAAGCTGCTCGCGGCCGCC | |
| | | | | ACCGCCGGGCCCCGGCCGTCCCTGGCTCCCCTCCT | |
| | | | | GCCTCGA | |
| HPV52 | SD2- | 1179 | 1180 | GTGTGCCCCGGCTGTGCACGGCTATAAACAACCCT | 2090 |
| | MYC_001_exon2 | | | GCAATGGAGGACCCTGAAGCAGCCTCCCGCGACG | |
| | | | | ATGCCCCTCAACGTTAGCTTCACCAACAGGAACTA | |
| | | | | TGACCTCGACTACG | |
| HPV52 | SD2- | 1181 | 1182 | GTGTGCCCCGGCTGTGCACGGCTATAAACAACCCT | 2091 |
| | MYC_001_exon3 | | | GCAATGGAGGACCCTGAAGAGGAGGAACAAGAA | |
| | | | | GATGAGGAAGAAATCGATGTTGTTTCTGTGGAAA | |
| | | | | AGAGGCAGGCTCCTGG | |
| HPV52 | SD2- | 1183 | 1184 | GTGTGCCCCGGCTGTGCACGGCTATAAACAACCCT | 2092 |
| | PVT1_002_exon3 | | | GCAATGGAGGACCCTGAAGCTGACCATACTCCCT | |
| | | | | GGAGCCTTCTCCCGAGGTGCGCGGGTGACCT | |
| HPV52 | SD2- | 1185 | 1186 | GTGTGCCCCGGCTGTGCACGGCTATAAACAACCCT | 2093 |
| | PVT1_004_exon1 | | | GCAATGGAGGACCCTGAAGTCTGAGCCTGATGGA | |
| | | | | TTTACAGTGATCTTCAGTGGTCTGGGG | |
| HPV52 | SD2- | 1187 | 1188 | GTGTGCCCCGGCTGTGCACGGCTATAAACAACCCT | 2094 |
| | PVT1_005_exon1 | | | GCAATGGAGGACCCTGAAGCTCCGGGCAGAGCGC | |
| | | | | GTGTGGCGGCCGAGCACATGGGCCCGCGGGCCGG | |
| | | | | GCGGGCTCGGGGCGGCCGGGACGAGGAGGGGCG | |
| | | | | ACG | |
| HPV56 | bkpt1- | 1189 | 1190 | TGAAAAAAGATAGTAGATGTATTGCAGATCATAT | 2095 |
| | MYC_001_exon1 | | | AGAATATTGGAAAGCTGTGCGACATGCTGCTCGC | |
| | | | | GGCCGCCACCGCCGGGCCCCGGCCGTCCCTGGCTC | |
| | | | | CCCTCCTGCCTCGA | |
| HPV56 | bkpt1- | 1191 | 1192 | TGAAAAAAGATAGTAGATGTATTGCAGATCATAT | 2096 |
| | MYC_001_exon2 | | | AGAATATTGGAAAGCTGTGCGACATGCAGCCTCC | |
| | | | | CGCGACGATGCCCCTCAACGTTAGCTTCACCAACA | |
| | | | | GGAACTATGACCTCGACTACG | |
| HPV56 | bkpt1- | 1193 | 1194 | TGAAAAAAGATAGTAGATGTATTGCAGATCATAT | 2097 |
| | MYC_001_exon3 | | | AGAATATTGGAAAGCTGTGCGACATGAGGAGGAA | |
| | | | | CAAGAAGATGAGGAAGAAATCGATGTTGTTTCTG | |
| | | | | TGGAAAAGAGGCAGGCTCCTGG | |
| HPV56 | bkpt1- | 1195 | 1196 | TGAAAAAAGATAGTAGATGTATTGCAGATCATAT | 2098 |
| | PVT1_002_exon3 | | | AGAATATTGGAAAGCTGTGCGACATGCTGACCAT | |
| | | | | ACTCCCTGGAGCCTTCTCCCGAGGTGCGCGGGTGA | |
| | | | | CCT | |
| HPV56 | bkpt1- | 1197 | 1198 | TGAAAAAAGATAGTAGATGTATTGCAGATCATAT | 2099 |
| | PVT1_004_exon1 | | | AGAATATTGGAAAGCTGTGCGACATGTCTGAGCC | |
| | | | | TGATGGATTTACAGTGATCTTCAGTGGTCTGGGG | |
| HPV56 | bkpt1- | 1199 | 1200 | TGAAAAAAGATAGTAGATGTATTGCAGATCATAT | 2100 |
| | PVT1_005_exon1 | | | AGAATATTGGAAAGCTGTGCGACATGCTCCGGGC | |
| | | | | AGAGCGCGTGTGGCGGCCGAGCACATGGGCCCGC | |
| | | | | GGGCCGGGCGGGCTCGGGGCGGCCGGGACGAGG | |
| | | | | AGGGGCGACG | |
| HPV56 | bkpt2- | 1201 | 1202 | ACTACGCCTGTAGCTGCTCGCGGCCGCCACCGCCG | 2101 |
| | MYC_001_exon1 | | | GGCCCCGGCCGTCCCTGGCTCCCCTCCTGCCTCGA | |
| HPV56 | bkpt2- | 1203 | 1204 | ACTACGCCTGTAGCAGCCTCCCGCGACGATGCCCC | 2102 |
| | MYC_001_exon2 | | | TCAACGTTAGCTTCACCAACAGGAACTATGACCTC | |
| | | | | GACTACG | |
| HPV56 | bkpt2- | 1205 | 1206 | ACTACGCCTGTAGAGGAGGAACAAGAAGATGAGG | 2103 |
| | MYC_001_exon3 | | | AAGAAATCGATGTTGTTTCTGTGGAAAAGAGGCA | |
| | | | | GGCTCCTGG | |
| HPV56 | bkpt2- | 1207 | 1208 | ACTACGCCTGTAGCTGACCATACTCCCTGGAGCCT | 2104 |
| | PVT1_002_exon3 | | | TCTCCCGAGGTGCGCGGGTGACCT | |
| HPV56 | bkpt2- | 1209 | 1210 | ACTACGCCTGTAGTCTGAGCCTGATGGATTTACAG | 2105 |
| | PVT1_004_exon1 | | | TGATCTTCAGTGGTCTGGGG | |
| HPV56 | bkpt2- | 1211 | 1212 | ACTACGCCTGTAGCTCCGGGCAGAGCGCGTGTGG | 2106 |
| | PVT1_005_exon1 | | | CGGCCGAGCACATGGGCCCGCGGGCCGGGCGGGC | |
| | | | | TCGGGGCGGCCGGGACGAGGAGGGGCGACG | |
| HPV56 | SD2- | 1213 | 1214 | GCGCATCAAGTAACTAACTGCAATGGCGTCACCT | 2107 |
| | MYC_001_exon1 | | | GAAGCTGCTCGCGGCCGCCACCGCCGGGCCCCGG | |
| | | | | CCGTCCCTGGCTCCCCTCCTGCCTCGA | |
| HPV56 | SD2- | 1215 | 1216 | GCGCATCAAGTAACTAACTGCAATGGCGTCACCT | 2108 |
| | MYC_001_exon2 | | | GAAGCAGCCTCCCGCGACGATGCCCCTCAACGTT | |
| | | | | AGCTTCACCAACAGGAACTATGACCTCGACTACG | |
| HPV56 | SD2- | 1217 | 1218 | GCGCATCAAGTAACTAACTGCAATGGCGTCACCT | 2109 |
| | MYC_001_exon3 | | | GAAGAGGAGGAACAAGAAGATGAGGAAGAAATC | |
| | | | | GATGTTGTTTCTGTGGAAAAGAGGCAGGCTCCTGG | |
| HPV56 | SD2- | 1219 | 1220 | GCGCATCAAGTAACTAACTGCAATGGCGTCACCT | 2110 |
| | PVT1_002_exon3 | | | GAAGCTGACCATACTCCCTGGAGCCTTCTCCCGAG | |
| | | | | GTGCGCGGGTGACCT | |
| HPV56 | SD2- | 1221 | 1222 | GCGCATCAAGTAACTAACTGCAATGGCGTCACCT | 2111 |
| | PVT1_004_exon1 | | | GAAGTCTGAGCCTGATGGATTTACAGTGATCTTCA | |
| | | | | GTGGTCTGGGG | |
| HPV56 | SD2- | 1223 | 1224 | GCGCATCAAGTAACTAACTGCAATGGCGTCACCT | 2112 |
| | PVT1_005_exon1 | | | GAAGCTCCGGGCAGAGCGCGTGTGGCGGCCGAGC | |
| | | | | ACATGGGCCCGCGGGCCGGGCGGGCTCGGGGCGG | |
| | | | | CCGGGACGAGGAGGGGCGACG | |
| HPV58 | bkpt1- | 1225 | 1226 | GCTGATAAAAATGATTTAACATCACAAATTGAAC | 2113 |
| | MYC_001_exon1 | | | ATTGGAAACTAATACGCATGGCTGCTCGCGGCCG | |
| | | | | CCACCGCCGGGCCCCGGCCGTCCCTGGCTCCCCTC | |
| | | | | CTGCCTCGA | |
| HPV58 | bkpt1- | 1227 | 1228 | GCTGATAAAAATGATTTAACATCACAAATTGAAC | 2114 |
| | MYC_001_exon2 | | | ATTGGAAACTAATACGCATGGCAGCCTCCCGCGA | |
| | | | | CGATGCCCCTCAACGTTAGCTTCACCAACAGGAAC | |
| | | | | TATGACCTCGACTACG | |
| HPV58 | bkpt1- | 1229 | 1230 | GCTGATAAAAATGATTTAACATCACAAATTGAAC | 2115 |
| | MYC_001_exon3 | | | ATTGGAAACTAATACGCATGGAGGAGGAACAAGA | |
| | | | | AGATGAGGAAGAAATCGATGTTGTTTCTGTGGAA | |
| | | | | AAGAGGCAGGCTCCTGG | |
| HPV58 | bkpt1- | 1231 | 1232 | GCTGATAAAAATGATTTAACATCACAAATTGAAC | 2116 |
| | PVT1_002_exon3 | | | ATTGGAAACTAATACGCATGGCTGACCATACTCCC | |
| | | | | TGGAGCCTTCTCCCGAGGTGCGCGGGTGACCT | |
| HPV58 | bkpt1- | 1233 | 1234 | GCTGATAAAAATGATTTAACATCACAAATTGAAC | 2117 |
| | PVT1_004_exon1 | | | ATTGGAAACTAATACGCATGGTCTGAGCCTGATG | |
| | | | | GATTTACAGTGATCTTCAGTGGTCTGGGG | |
| HPV58 | bkpt1- | 1235 | 1236 | GCTGATAAAAATGATTTAACATCACAAATTGAAC | 2118 |
| | PVT1_005_exon1 | | | ATTGGAAACTAATACGCATGGCTCCGGGCAGAGC | |
| | | | | GCGTGTGGCGGCCGAGCACATGGGCCCGCGGGCC | |
| | | | | GGGCGGGCTCGGGGCGGCCGGGACGAGGAGGGG | |
| | | | | CGACG | |
| HPV58 | bkpt2- | 1237 | 1238 | GTACATACAAAGGGCGGAACGTGTGTAGTTCTAA | 2119 |
| | MYC_001_exon1 | | | AGTTTCACCTATCGCTGCTCGCGGCCGCCACCGCC | |
| | | | | GGGCCCCGGCCGTCCCTGGCTCCCCTCCTGCCTCG | |
| | | | | A | |
| HPV58 | bkpt2- | 1239 | 1240 | GTACATACAAAGGGCGGAACGTGTGTAGTTCTAA | 2120 |
| | MYC_001_exon2 | | | AGTTTCACCTATCGCAGCCTCCCGCGACGATGCCC | |
| | | | | CTCAACGTTAGCTTCACCAACAGGAACTATGACCT | |
| | | | | CGACTACG | |
| HPV58 | bkpt2- | 1241 | 1242 | GTACATACAAAGGGCGGAACGTGTGTAGTTCTAA | 2121 |
| | MYC_001_exon3 | | | AGTTTCACCTATCGAGGAGGAACAAGAAGATGAG | |
| | | | | GAAGAAATCGATGTTGTTTCTGTGGAAAAGAGGC | |
| | | | | AGGCTCCTGG | |
| HPV58 | bkpt2- | 1243 | 1244 | GTACATACAAAGGGCGGAACGTGTGTAGTTCTAA | 2122 |
| | PVT1_002_exon3 | | | AGTTTCACCTATCGCTGACCATACTCCCTGGAGCC | |
| | | | | TTCTCCCGAGGTGCGCGGGTGACCT | |
| HPV58 | bkpt2- | 1245 | 1246 | GTACATACAAAGGGCGGAACGTGTGTAGTTCTAA | 2123 |
| | PVT1_004_exon1 | | | AGTTTCACCTATCGTCTGAGCCTGATGGATTTACA | |
| | | | | GTGATCTTCAGTGGTCTGGGG | |
| HPV58 | bkpt2- | 1247 | 1248 | GTACATACAAAGGGCGGAACGTGTGTAGTTCTAA | 2124 |
| | PVT1_005_exon1 | | | AGTTTCACCTATCGCTCCGGGCAGAGCGCGTGTGG | |
| | | | | CGGCCGAGCACATGGGCCCGCGGGCCGGGCGGGC | |
| | | | | TCGGGGCGGCCGGGACGAGGAGGGGCGACG | |
| HPV58 | SD2- | 1249 | 1250 | GTGTGCCCTAGCTGTGCACAGCAATAAACACCATC | 2125 |
| | MYC_001_exon1 | | | TGCAATGGATGACCCTGAAGCTGCTCGCGGCCGC | |
| | | | | CACCGCCGGGCCCCGGCCGTCCCTGGCTCCCCTCC | |
| | | | | TGCCTCGA | |
| HPV58 | SD2- | 1251 | 1252 | GTGTGCCCTAGCTGTGCACAGCAATAAACACCATC | 2126 |
| | MYC_001_exon2 | | | TGCAATGGATGACCCTGAAGCAGCCTCCCGCGAC | |
| | | | | GATGCCCCTCAACGTTAGCTTCACCAACAGGAACT | |
| | | | | ATGACCTCGACTACG | |
| HPV58 | SD2- | 1253 | 1254 | GTGTGCCCTAGCTGTGCACAGCAATAAACACCATC | 2127 |
| | MYC_001_exon3 | | | TGCAATGGATGACCCTGAAGAGGAGGAACAAGAA | |
| | | | | GATGAGGAAGAAATCGATGTTGTTTCTGTGGAAA | |
| | | | | AGAGGCAGGCTCCTGG | |
| HPV58 | SD2- | 1255 | 1256 | GTGTGCCCTAGCTGTGCACAGCAATAAACACCATC | 2128 |
| | PVT1_002_exon3 | | | TGCAATGGATGACCCTGAAGCTGACCATACTCCCT | |
| | | | | GGAGCCTTCTCCCGAGGTGCGCGGGTGACCT | |
| HPV58 | SD2- | 1257 | 1258 | GTGTGCCCTAGCTGTGCACAGCAATAAACACCATC | 2129 |
| | PVT1_004_exon1 | | | TGCAATGGATGACCCTGAAGTCTGAGCCTGATGG | |
| | | | | ATTTACAGTGATCTTCAGTGGTCTGGGG | |
| HPV58 | SD2- | 1259 | 1260 | GTGTGCCCTAGCTGTGCACAGCAATAAACACCATC | 2130 |
| | PVT1_005_exon1 | | | TGCAATGGATGACCCTGAAGCTCCGGGCAGAGCG | |
| | | | | CGTGTGGCGGCCGAGCACATGGGCCCGCGGGCCG | |
| | | | | GGCGGGCTCGGGGCGGCCGGGACGAGGAGGGGC | |
| | | | | GACG | |
| HPV59 | bkpt1- | 1261 | 1262 | ATTAGAACATTATGAAAACGATAGTAAAGACATT | 2131 |
| | MYC_001_exon1 | | | AATGAACACATAAACTATTGGAAACTGGTGCGTA | |
| | | | | TGGCTGCTCGCGGCCGCCACCGCCGGGCCCCGGC | |
| | | | | CGTCCCTGGCTCCCCTCCTGCCTCGA | |
| HPV59 | bkpt1- | 1263 | 1264 | ATTAGAACATTATGAAAACGATAGTAAAGACATT | 2132 |
| | MYC_001_exon2 | | | AATGAACACATAAACTATTGGAAACTGGTGCGTA | |
| | | | | TGGCAGCCTCCCGCGACGATGCCCCTCAACGTTAG | |
| | | | | CTTCACCAACAGGAACTATGACCTCGACTACG | |
| HPV59 | bkpt1- | 1265 | 1266 | ATTAGAACATTATGAAAACGATAGTAAAGACATT | 2133 |
| | MYC_001_exon3 | | | AATGAACACATAAACTATTGGAAACTGGTGCGTA | |
| | | | | TGGAGGAGGAACAAGAAGATGAGGAAGAAATCG | |
| | | | | ATGTTGTTTCTGTGGAAAAGAGGCAGGCTCCTGG | |
| HPV59 | bkpt1- | 1267 | 1268 | ATTAGAACATTATGAAAACGATAGTAAAGACATT | 2134 |
| | PVT1_002_exon3 | | | AATGAACACATAAACTATTGGAAACTGGTGCGTA | |
| | | | | TGGCTGACCATACTCCCTGGAGCCTTCTCCCGAGG | |
| | | | | TGCGCGGGTGACCT | |
| HPV59 | bkpt1- | 1269 | 1270 | ATTAGAACATTATGAAAACGATAGTAAAGACATT | 2135 |
| | PVT1_004_exon1 | | | AATGAACACATAAACTATTGGAAACTGGTGCGTA | |
| | | | | TGGTCTGAGCCTGATGGATTTACAGTGATCTTCAG | |
| | | | | TGGTCTGGGG | |
| HPV59 | bkpt1- | 1271 | 1272 | ATTAGAACATTATGAAAACGATAGTAAAGACATT | 2136 |
| | PVT1_005_exon1 | | | AATGAACACATAAACTATTGGAAACTGGTGCGTA | |
| | | | | TGGCTCCGGGCAGAGCGCGTGTGGCGGCCGAGCA | |
| | | | | CATGGGCCCGCGGGCCGGGCGGGCTCGGGGCGGC | |
| | | | | CGGGACGAGGAGGGGCGACG | |
| HPV59 | bkpt2- | 1273 | 1274 | CAACCCGCGACGGCACATCCCTTGCAGTAACACT | 2137 |
| | MYC_001_exon1 | | | ACGCCTATAACTGCTCGCGGCCGCCACCGCCGGG | |
| | | | | CCCCGGCCGTCCCTGGCTCCCCTCCTGCCTCGA | |
| HPV59 | bkpt2- | 1275 | 1276 | CAACCCGCGACGGCACATCCCTTGCAGTAACACT | 2138 |
| | MYC_001_exon2 | | | ACGCCTATAACAGCCTCCCGCGACGATGCCCCTCA | |
| | | | | ACGTTAGCTTCACCAACAGGAACTATGACCTCGAC | |
| | | | | TACG | |
| HPV59 | bkpt2- | 1277 | 1278 | CAACCCGCGACGGCACATCCCTTGCAGTAACACT | 2139 |
| | MYC_001_exon3 | | | ACGCCTATAAAGGAGGAACAAGAAGATGAGGAA | |
| | | | | GAAATCGATGTTGTTTCTGTGGAAAAGAGGCAGG | |
| | | | | CTCCTGG | |
| HPV59 | bkpt2- | 1279 | 1280 | CAACCCGCGACGGCACATCCCTTGCAGTAACACT | 2140 |
| | PVT1_002_exon3 | | | ACGCCTATAACTGACCATACTCCCTGGAGCCTTCT | |
| | | | | CCCGAGGTGCGCGGGTGACCT | |
| HPV59 | bkpt2- | 1281 | 1282 | CAACCCGCGACGGCACATCCCTTGCAGTAACACT | 2141 |
| | PVT1_004_exon1 | | | ACGCCTATAATCTGAGCCTGATGGATTTACAGTGA | |
| | | | | TCTTCAGTGGTCTGGGG | |
| HPV59 | bkpt2- | 1283 | 1284 | CAACCCGCGACGGCACATCCCTTGCAGTAACACT | 2142 |
| | PVT1_005_exon1 | | | ACGCCTATAACTCCGGGCAGAGCGCGTGTGGCGG | |
| | | | | CCGAGCACATGGGCCCGCGGGCCGGGCGGGCTCG | |
| | | | | GGGCGGCCGGGACGAGGAGGGGCGACG | |
| HPV59 | SD2- | 1285 | 1286 | GCAGCAAACCAGTAACCTGCAATGGCCGATTCGG | 2143 |
| | MYC_001_exon1 | | | AAGCTGCTCGCGGCCGCCACCGCCGGGCCCCGGC | |
| | | | | CGTCCCTGGCTCCCCTCCTGCCTCGA | |
| HPV59 | SD2- | 1287 | 1288 | GCAGCAAACCAGTAACCTGCAATGGCCGATTCGG | 2144 |
| | MYC_001_exon2 | | | AAGCAGCCTCCCGCGACGATGCCCCTCAACGTTA | |
| | | | | GCTTCACCAACAGGAACTATGACCTCGACTACG | |
| HPV59 | SD2- | 1289 | 1290 | GCAGCAAACCAGTAACCTGCAATGGCCGATTCGG | 2145 |
| | MYC_001_exon3 | | | AAGAGGAGGAACAAGAAGATGAGGAAGAAATCG | |
| | | | | ATGTTGTTTCTGTGGAAAAGAGGCAGGCTCCTGG | |
| HPV59 | SD2- | 1291 | 1292 | GCAGCAAACCAGTAACCTGCAATGGCCGATTCGG | 2146 |
| | PVT1_002_exon3 | | | AAGCTGACCATACTCCCTGGAGCCTTCTCCCGAGG | |
| | | | | TGCGCGGGTGACCT | |
| HPV59 | SD2- | 1293 | 1294 | GCAGCAAACCAGTAACCTGCAATGGCCGATTCGG | 2147 |
| | PVT1_004_exon1 | | | AAGTCTGAGCCTGATGGATTTACAGTGATCTTCAG | |
| | | | | TGGTCTGGGG | |
| HPV59 | SD2- | 1295 | 1296 | GCAGCAAACCAGTAACCTGCAATGGCCGATTCGG | 2148 |
| | PVT1_005_exon1 | | | AAGCTCCGGGCAGAGCGCGTGTGGCGGCCGAGCA | |
| | | | | CATGGGCCCGCGGGCCGGGCGGGCTCGGGGCGGC | |
| | | | | CGGGACGAGGAGGGGCGACG | |
| HPV66 | bkpt1- | 1297 | 1298 | TATGAAAAAGATAGTAAATGCATTATAGATCACA | 2149 |
| | MYC_001_exon1 | | | TAGACTATTGGAAAGCTGTACGACATGCTGCTCGC | |
| | | | | GGCCGCCACCGCCGGGCCCCGGCCGTCCCTGGCTC | |
| | | | | CCCTCCTGCCTCGA | |
| HPV66 | bkpt1- | 1299 | 1300 | TATGAAAAAGATAGTAAATGCATTATAGATCACA | 2150 |
| | MYC_001_exon2 | | | TAGACTATTGGAAAGCTGTACGACATGCAGCCTCC | |
| | | | | CGCGACGATGCCCCTCAACGTTAGCTTCACCAACA | |
| | | | | GGAACTATGACCTCGACTACG | |
| HPV66 | bkpt1- | 1301 | 1302 | TATGAAAAAGATAGTAAATGCATTATAGATCACA | 2151 |
| | MYC_001_exon3 | | | TAGACTATTGGAAAGCTGTACGACATGAGGAGGA | |
| | | | | ACAAGAAGATGAGGAAGAAATCGATGTTGTTTCT | |
| | | | | GTGGAAAAGAGGCAGGCTCCTGG | |
| HPV66 | bkpt1- | 1303 | 1304 | TATGAAAAAGATAGTAAATGCATTATAGATCACA | 2152 |
| | PVT1_002_exon3 | | | TAGACTATTGGAAAGCTGTACGACATGCTGACCAT | |
| | | | | ACTCCCTGGAGCCTTCTCCCGAGGTGCGCGGGTGA | |
| | | | | CCT | |
| HPV66 | bkpt1- | 1305 | 1306 | TATGAAAAAGATAGTAAATGCATTATAGATCACA | 2153 |
| | PVT1_004_exon1 | | | TAGACTATTGGAAAGCTGTACGACATGTCTGAGCC | |
| | | | | TGATGGATTTACAGTGATCTTCAGTGGTCTGGGG | |
| HPV66 | bkpt1- | 1307 | 1308 | TATGAAAAAGATAGTAAATGCATTATAGATCACA | 2154 |
| | PVT1_005_exon1 | | | TAGACTATTGGAAAGCTGTACGACATGCTCCGGG | |
| | | | | CAGAGCGCGTGTGGCGGCCGAGCACATGGGCCCG | |
| | | | | CGGGCCGGGCGGGCTCGGGGCGGCCGGGACGAGG | |
| | | | | AGGGGCGACG | |
| HPV66 | bkpt2- | 1309 | 1310 | GGTGATAAAACTACGCCTGTAACTGCTCGCGGCC | 2155 |
| | MYC_001_exon1 | | | GCCACCGCCGGGCCCCGGCCGTCCCTGGCTCCCCT | |
| | | | | CCTGCCTCGA | |
| HPV66 | bkpt2- | 1311 | 1312 | GGTGATAAAACTACGCCTGTAACAGCCTCCCGCG | 2156 |
| | MYC_001_exon2 | | | ACGATGCCCCTCAACGTTAGCTTCACCAACAGGA | |
| | | | | ACTATGACCTCGACTACG | |
| HPV66 | bkpt2- | 1313 | 1314 | GGTGATAAAACTACGCCTGTAAAGGAGGAACAAG | 2157 |
| | MYC_001_exon3 | | | AAGATGAGGAAGAAATCGATGTTGTTTCTGTGGA | |
| | | | | AAAGAGGCAGGCTCCTGG | |
| HPV66 | bkpt2- | 1315 | 1316 | GGTGATAAAACTACGCCTGTAACTGACCATACTCC | 2158 |
| | PVT1_002_exon3 | | | CTGGAGCCTTCTCCCGAGGTGCGCGGGTGACCT | |
| HPV66 | bkpt2- | 1317 | 1318 | GGTGATAAAACTACGCCTGTAATCTGAGCCTGATG | 2159 |
| | PVT1_004_exon1 | | | GATTTACAGTGATCTTCAGTGGTCTGGGG | |
| HPV66 | bkpt2- | 1319 | 1320 | GGTGATAAAACTACGCCTGTAACTCCGGGCAGAG | 2160 |
| | PVT1_005_exon1 | | | CGCGTGTGGCGGCCGAGCACATGGGCCCGCGGGC | |
| | | | | CGGGCGGGCTCGGGGCGGCCGGGACGAGGAGGG | |
| | | | | GCGACG | |
| HPV66 | SD2- | 1321 | 1322 | GCGCATCATCTAAATAACTGCAATGGCATCACCTG | 2161 |
| | MYC_001_exon1 | | | AAGCTGCTCGCGGCCGCCACCGCCGGGCCCCGGC | |
| | | | | CGTCCCTGGCTCCCCTCCTGCCTCGA | |
| HPV66 | SD2- | 1323 | 1324 | GCGCATCATCTAAATAACTGCAATGGCATCACCTG | 2162 |
| | MYC_001_exon2 | | | AAGCAGCCTCCCGCGACGATGCCCCTCAACGTTA | |
| | | | | GCTTCACCAACAGGAACTATGACCTCGACTACG | |
| HPV66 | SD2- | 1325 | 1326 | GCGCATCATCTAAATAACTGCAATGGCATCACCTG | 2163 |
| | MYC_001_exon3 | | | AAGAGGAGGAACAAGAAGATGAGGAAGAAATCG | |
| | | | | ATGTTGTTTCTGTGGAAAAGAGGCAGGCTCCTGG | |
| HPV66 | SD2- | 1327 | 1328 | GCGCATCATCTAAATAACTGCAATGGCATCACCTG | 2164 |
| | PVT1_002_exon3 | | | AAGCTGACCATACTCCCTGGAGCCTTCTCCCGAGG | |
| | | | | TGCGCGGGTGACCT | |
| HPV66 | SD2- | 1329 | 1330 | GCGCATCATCTAAATAACTGCAATGGCATCACCTG | 2165 |
| | PVT1_004_exon1 | | | AAGTCTGAGCCTGATGGATTTACAGTGATCTTCAG | |
| | | | | TGGTCTGGGG | |
| HPV66 | SD2- | 1331 | 1332 | GCGCATCATCTAAATAACTGCAATGGCATCACCTG | 2166 |
| | PVT1_005_exon1 | | | AAGCTCCGGGCAGAGCGCGTGTGGCGGCCGAGCA | |
| | | | | CATGGGCCCGCGGGCCGGGCGGGCTCGGGGCGGC | |
| | | | | CGGGACGAGGAGGGGCGACG | |
| HPV68 | bkpt1- | 1333 | 1334 | ATTAACTATTGGAATTGTGTGCGACTGGCTGCTCG | 2167 |
| | MYC_001_exon1 | | | CGGCCGCCACCGCCGGGCCCCGGCCGTCCCTGGCT | |
| | | | | CCCCTCCTGCCTCGA | |
| HPV68 | bkpt1- | 1335 | 1336 | ATTAACTATTGGAATTGTGTGCGACTGGCAGCCTC | 2168 |
| | MYC_001_exon2 | | | CCGCGACGATGCCCCTCAACGTTAGCTTCACCAAC | |
| | | | | AGGAACTATGACCTCGACTACG | |
| HPV68 | bkpt1- | 1337 | 1338 | ATTAACTATTGGAATTGTGTGCGACTGGAGGAGG | 2169 |
| | MYC_001_exon3 | | | AACAAGAAGATGAGGAAGAAATCGATGTTGTTTC | |
| | | | | TGTGGAAAAGAGGCAGGCTCCTGG | |
| HPV68 | bkpt1- | 1339 | 1340 | ATTAACTATTGGAATTGTGTGCGACTGGCTGACCA | 2170 |
| | PVT1_002_exon3 | | | TACTCCCTGGAGCCTTCTCCCGAGGTGCGCGGGTG | |
| | | | | ACCT | |
| HPV68 | bkpt1- | 1341 | 1342 | ATTAACTATTGGAATTGTGTGCGACTGGTCTGAGC | 2171 |
| | PVT1_004_exon1 | | | CTGATGGATTTACAGTGATCTTCAGTGGTCTGGGG | |
| HPV68 | bkpt1- | 1343 | 1344 | ATTAACTATTGGAATTGTGTGCGACTGGCTCCGGG | 2172 |
| | PVT1_005_exon1 | | | CAGAGCGCGTGTGGCGGCCGAGCACATGGGCCCG | |
| | | | | CGGGCCGGGCGGGCTCGGGGCGGCCGGGACGAGG | |
| | | | | AGGGGCGACG | |
| HPV68 | bkpt2- | 1345 | 1346 | AAGACGGAGCCTTTGTTGTGGTGACACTACACCTA | 2173 |
| | MYC_001_exon1 | | | TAGCTGCTCGCGGCCGCCACCGCCGGGCCCCGGC | |
| | | | | CGTCCCTGGCTCCCCTCCTGCCTCGA | |
| HPV68 | bkpt2- | 1347 | 1348 | AAGACGGAGCCTTTGTTGTGGTGACACTACACCTA | 2174 |
| | MYC_001_exon2 | | | TAGCAGCCTCCCGCGACGATGCCCCTCAACGTTAG | |
| | | | | CTTCACCAACAGGAACTATGACCTCGACTACG | |
| HPV68 | bkpt2- | 1349 | 1350 | AAGACGGAGCCTTTGTTGTGGTGACACTACACCTA | 2175 |
| | MYC_001_exon3 | | | TAGAGGAGGAACAAGAAGATGAGGAAGAAATCG | |
| | | | | ATGTTGTTTCTGTGGAAAAGAGGCAGGCTCCTGG | |
| HPV68 | bkpt2- | 1351 | 1352 | AAGACGGAGCCTTTGTTGTGGTGACACTACACCTA | 2176 |
| | PVT1_002_exon3 | | | TAGCTGACCATACTCCCTGGAGCCTTCTCCCGAGG | |
| | | | | TGCGCGGGTGACCT | |
| HPV68 | bkpt2- | 1353 | 1354 | AAGACGGAGCCTTTGTTGTGGTGACACTACACCTA | 2177 |
| | PVT1_004_exon1 | | | TAGTCTGAGCCTGATGGATTTACAGTGATCTTCAG | |
| | | | | TGGTCTGGGG | |
| HPV68 | bkpt2- | 1355 | 1356 | AAGACGGAGCCTTTGTTGTGGTGACACTACACCTA | 2178 |
| | PVT1_005_exon1 | | | TAGCTCCGGGCAGAGCGCGTGTGGCGGCCGAGCA | |
| | | | | CATGGGCCCGCGGGCCGGGCGGGCTCGGGGCGGC | |
| | | | | CGGGACGAGGAGGGGCGACG | |
| HPV68 | SD2- | 1357 | 1358 | ACCCAGTAATCTGCAATGGCCAATTGTGAAGCTGC | 2179 |
| | MYC_001_exon1 | | | TCGCGGCCGCCACCGCCGGGCCCCGGCCGTCCCTG | |
| | | | | GCTCCCCTCCTGCCTCGA | |
| HPV68 | SD2- | 1359 | 1360 | ACCCAGTAATCTGCAATGGCCAATTGTGAAGCAG | 2180 |
| | MYC_001_exon2 | | | CCTCCCGCGACGATGCCCCTCAACGTTAGCTTCAC | |
| | | | | CAACAGGAACTATGACCTCGACTACG | |
| HPV68 | SD2- | 1361 | 1362 | ACCCAGTAATCTGCAATGGCCAATTGTGAAGAGG | 2181 |
| | MYC_001_exon3 | | | AGGAACAAGAAGATGAGGAAGAAATCGATGTTGT | |
| | | | | TTCTGTGGAAAAGAGGCAGGCTCCTGG | |
| HPV68 | SD2- | 1363 | 1364 | ACCCAGTAATCTGCAATGGCCAATTGTGAAGCTG | 2182 |
| | PVT1_002_exon3 | | | ACCATACTCCCTGGAGCCTTCTCCCGAGGTGCGCG | |
| | | | | GGTGACCT | |
| HPV68 | SD2- | 1365 | 1366 | ACCCAGTAATCTGCAATGGCCAATTGTGAAGTCTG | 2183 |
| | PVT1_004_exon1 | | | AGCCTGATGGATTTACAGTGATCTTCAGTGGTCTG | |
| | | | | GGG | |
| HPV68 | SD2- | 1367 | 1368 | ACCCAGTAATCTGCAATGGCCAATTGTGAAGCTCC | 2184 |
| | PVT1_005_exon1 | | | GGGCAGAGCGCGTGTGGCGGCCGAGCACATGGGC | |
| | | | | CCGCGGGCCGGGCGGGCTCGGGGCGGCCGGGACG | |
| | | | | AGGAGGGGCGACG | |
| HPV73 | bkpt1- | 1369 | 1370 | GTGATCATATTGATCATTGGAAACACGTGCGACAT | 2185 |
| | MYC_001_exon1 | | | GCTGCTCGCGGCCGCCACCGCCGGGCCCCGGCCG | |
| | | | | TCCCTGGCTCCCCTCCTGCCTCGA | |
| HPV73 | bkpt1- | 1371 | 1372 | GTGATCATATTGATCATTGGAAACACGTGCGACAT | 2186 |
| | MYC_001_exon2 | | | GCAGCCTCCCGCGACGATGCCCCTCAACGTTAGCT | |
| | | | | TCACCAACAGGAACTATGACCTCGACTACG | |
| HPV73 | bkpt1- | 1373 | 1374 | GTGATCATATTGATCATTGGAAACACGTGCGACAT | 2187 |
| | MYC_001_exon3 | | | GAGGAGGAACAAGAAGATGAGGAAGAAATCGAT | |
| | | | | GTTGTTTCTGTGGAAAAGAGGCAGGCTCCTGG | |
| HPV73 | bkpt1- | 1375 | 1376 | GTGATCATATTGATCATTGGAAACACGTGCGACAT | 2188 |
| | PVT1_002_exon3 | | | GCTGACCATACTCCCTGGAGCCTTCTCCCGAGGTG | |
| | | | | CGCGGGTGACCT | |
| HPV73 | bkpt1- | 1377 | 1378 | GTGATCATATTGATCATTGGAAACACGTGCGACAT | 2189 |
| | PVT1_004_exon1 | | | GTCTGAGCCTGATGGATTTACAGTGATCTTCAGTG | |
| | | | | GTCTGGGG | |
| HPV73 | bkpt1- | 1379 | 1380 | GTGATCATATTGATCATTGGAAACACGTGCGACAT | 2190 |
| | PVT1_005_exon1 | | | GCTCCGGGCAGAGCGCGTGTGGCGGCCGAGCACA | |
| | | | | TGGGCCCGCGGGCCGGGCGGGCTCGGGGCGGCCG | |
| | | | | GGACGAGGAGGGGCGACG | |
| HPV73 | bkpt2- | 1381 | 1382 | CCTGTACCCAGTGTACTACACATAATGTTGCGCCA | 2191 |
| | MYC_001_exon1 | | | ATAGCTGCTCGCGGCCGCCACCGCCGGGCCCCGG | |
| | | | | CCGTCCCTGGCTCCCCTCCTGCCTCGA | |
| HPV73 | bkpt2- | 1383 | 1384 | CCTGTACCCAGTGTACTACACATAATGTTGCGCCA | 2192 |
| | MYC_001_exon2 | | | ATAGCAGCCTCCCGCGACGATGCCCCTCAACGTTA | |
| | | | | GCTTCACCAACAGGAACTATGACCTCGACTACG | |
| HPV73 | bkpt2- | 1385 | 1386 | CCTGTACCCAGTGTACTACACATAATGTTGCGCCA | 2193 |
| | MYC_001_exon3 | | | ATAGAGGAGGAACAAGAAGATGAGGAAGAAATC | |
| | | | | GATGTTGTTTCTGTGGAAAAGAGGCAGGCTCCTGG | |
| HPV73 | bkpt2- | 1387 | 1388 | CCTGTACCCAGTGTACTACACATAATGTTGCGCCA | 2194 |
| | PVT1_002_exon3 | | | ATAGCTGACCATACTCCCTGGAGCCTTCTCCCGAG | |
| | | | | GTGCGCGGGTGACCT | |
| HPV73 | bkpt2- | 1389 | 1390 | CCTGTACCCAGTGTACTACACATAATGTTGCGCCA | 2195 |
| | PVT1_004_exon1 | | | ATAGTCTGAGCCTGATGGATTTACAGTGATCTTCA | |
| | | | | GTGGTCTGGGG | |
| HPV73 | bkpt2- | 1391 | 1392 | CCTGTACCCAGTGTACTACACATAATGTTGCGCCA | 2196 |
| | PVT1_005_exon1 | | | ATAGCTCCGGGCAGAGCGCGTGTGGCGGCCGAGC | |
| | | | | ACATGGGCCCGCGGGCCGGGCGGGCTCGGGGCGG | |
| | | | | CCGGGACGAGGAGGGGCGACG | |
| HPV73 | SD2- | 1393 | 1394 | GCCCCAACTGTTCCAGAAACCTATAAAAGAAGAT | 2197 |
| | MYC_001_exon1 | | | GGCTGATTCAGCTGCTCGCGGCCGCCACCGCCGG | |
| | | | | GCCCCGGCCGTCCCTGGCTCCCCTCCTGCCTCGA | |
| HPV73 | SD2- | 1395 | 1396 | GCCCCAACTGTTCCAGAAACCTATAAAAGAAGAT | 2198 |
| | MYC_001_exon2 | | | GGCTGATTCAGCAGCCTCCCGCGACGATGCCCCTC | |
| | | | | AACGTTAGCTTCACCAACAGGAACTATGACCTCG | |
| | | | | ACTACG | |
| HPV73 | SD2- | 1397 | 1398 | GCCCCAACTGTTCCAGAAACCTATAAAAGAAGAT | 2199 |
| | MYC_001_exon3 | | | GGCTGATTCAGAGGAGGAACAAGAAGATGAGGA | |
| | | | | AGAAATCGATGTTGTTTCTGTGGAAAAGAGGCAG | |
| | | | | GCTCCTGG | |
| HPV73 | SD2- | 1399 | 1400 | GCCCCAACTGTTCCAGAAACCTATAAAAGAAGAT | 2200 |
| | PVT1_002_exon3 | | | GGCTGATTCAGCTGACCATACTCCCTGGAGCCTTC | |
| | | | | TCCCGAGGTGCGCGGGTGACCT | |
| HPV73 | SD2- | 1401 | 1402 | GCCCCAACTGTTCCAGAAACCTATAAAAGAAGAT | 2201 |
| | PVT1_004_exon1 | | | GGCTGATTCAGTCTGAGCCTGATGGATTTACAGTG | |
| | | | | ATCTTCAGTGGTCTGGGG | |
| HPV73 | SD2- | 1403 | 1404 | GCCCCAACTGTTCCAGAAACCTATAAAAGAAGAT | 2202 |
| | PVT1_005_exon1 | | | GGCTGATTCAGCTCCGGGCAGAGCGCGTGTGGCG | |
| | | | | GCCGAGCACATGGGCCCGCGGGCCGGGCGGGCTC | |
| | | | | GGGGCGGCCGGGACGAGGAGGGGCGACG | |
| HPV82 | bkpt1- | 1405 | 1406 | GAACTGGACAGTGATAAATTAGTAGATCAAATTA | 2203 |
| | MYC_001_exon1 | | | ATTATTGGACGTTGGTACGATATGCTGCTCGCGGC | |
| | | | | CGCCACCGCCGGGCCCCGGCCGTCCCTGGCTCCCC | |
| | | | | TCCTGCCTCGA | |
| HPV82 | bkpt1- | 1407 | 1408 | GAACTGGACAGTGATAAATTAGTAGATCAAATTA | 2204 |
| | MYC_001_exon2 | | | ATTATTGGACGTTGGTACGATATGCAGCCTCCCGC | |
| | | | | GACGATGCCCCTCAACGTTAGCTTCACCAACAGG | |
| | | | | AACTATGACCTCGACTACG | |
| HPV82 | bkpt1- | 1409 | 1410 | GAACTGGACAGTGATAAATTAGTAGATCAAATTA | 2205 |
| | MYC_001_exon3 | | | ATTATTGGACGTTGGTACGATATGAGGAGGAACA | |
| | | | | AGAAGATGAGGAAGAAATCGATGTTGTTTCTGTG | |
| | | | | GAAAAGAGGCAGGCTCCTGG | |
| HPV82 | bkpt1- | 1411 | 1412 | GAACTGGACAGTGATAAATTAGTAGATCAAATTA | 2206 |
| | PVT1_002_exon3 | | | ATTATTGGACGTTGGTACGATATGCTGACCATACT | |
| | | | | CCCTGGAGCCTTCTCCCGAGGTGCGCGGGTGACCT | |
| HPV82 | bkpt1- | 1413 | 1414 | GAACTGGACAGTGATAAATTAGTAGATCAAATTA | 2207 |
| | PVT1_004_exon1 | | | ATTATTGGACGTTGGTACGATATGTCTGAGCCTGA | |
| | | | | TGGATTTACAGTGATCTTCAGTGGTCTGGGG | |
| HPV82 | bkpt1- | 1415 | 1416 | GAACTGGACAGTGATAAATTAGTAGATCAAATTA | 2208 |
| | PVT1_005_exon1 | | | ATTATTGGACGTTGGTACGATATGCTCCGGGCAGA | |
| | | | | GCGCGTGTGGCGGCCGAGCACATGGGCCCGCGGG | |
| | | | | CCGGGCGGGCTCGGGGCGGCCGGGACGAGGAGG | |
| | | | | GGCGACG | |
| HPV82 | bkpt2- | 1417 | 1418 | GGAACTGCAGGCCCAAACACCGGAGGGCACCTCA | 2209 |
| | MYC_001_exon1 | | | GTGCAACTAAAACTGCGTTTATAGCTGCTCGCGGC | |
| | | | | CGCCACCGCCGGGCCCCGGCCGTCCCTGGCTCCCC | |
| | | | | TCCTGCCTCGA | |
| HPV82 | bkpt2- | 1419 | 1420 | GGAACTGCAGGCCCAAACACCGGAGGGCACCTCA | 2210 |
| | MYC_001_exon2 | | | GTGCAACTAAAACTGCGTTTATAGCAGCCTCCCGC | |
| | | | | GACGATGCCCCTCAACGTTAGCTTCACCAACAGG | |
| | | | | AACTATGACCTCGACTACG | |
| HPV82 | bkpt2- | 1421 | 1422 | GGAACTGCAGGCCCAAACACCGGAGGGCACCTCA | 2211 |
| | MYC_001_exon3 | | | GTGCAACTAAAACTGCGTTTATAGAGGAGGAACA | |
| | | | | AGAAGATGAGGAAGAAATCGATGTTGTTTCTGTG | |
| | | | | GAAAAGAGGCAGGCTCCTGG | |
| HPV82 | bkpt2- | 1423 | 1424 | GGAACTGCAGGCCCAAACACCGGAGGGCACCTCA | 2212 |
| | PVT1_002_exon3 | | | GTGCAACTAAAACTGCGTTTATAGCTGACCATACT | |
| | | | | CCCTGGAGCCTTCTCCCGAGGTGCGCGGGTGACCT | |
| HPV82 | bkpt2- | 1425 | 1426 | GGAACTGCAGGCCCAAACACCGGAGGGCACCTCA | 2213 |
| | PVT1_004_exon1 | | | GTGCAACTAAAACTGCGTTTATAGTCTGAGCCTGA | |
| | | | | TGGATTTACAGTGATCTTCAGTGGTCTGGGG | |
| HPV82 | bkpt2- | 1427 | 1428 | GGAACTGCAGGCCCAAACACCGGAGGGCACCTCA | 2214 |
| | PVT1_005_exon1 | | | GTGCAACTAAAACTGCGTTTATAGCTCCGGGCAG | |
| | | | | AGCGCGTGTGGCGGCCGAGCACATGGGCCCGCGG | |
| | | | | GCCGGGCGGGCTCGGGGCGGCCGGGACGAGGAG | |
| | | | | GGGCGACG | |
| HPV82 | SD2- | 1429 | 1430 | CATCGGCAATGGACAGTGAAGCTGCTCGCGGCCG | 2215 |
| | MYC_001_exon1 | | | CCACCGCCGGGCCCCGGCCGTCCCTGGCTCCCCTC | |
| | | | | CTGCCTCGA | |
| HPV82 | SD2- | 1431 | 1432 | CATCGGCAATGGACAGTGAAGCAGCCTCCCGCGA | 2216 |
| | MYC_001_exon2 | | | CGATGCCCCTCAACGTTAGCTTCACCAACAGGAAC | |
| | | | | TATGACCTCGACTACG | |
| HPV82 | SD2- | 1433 | 1434 | CATCGGCAATGGACAGTGAAGAGGAGGAACAAG | 2217 |
| | MYC_001_exon3 | | | AAGATGAGGAAGAAATCGATGTTGTTTCTGTGGA | |
| | | | | AAAGAGGCAGGCTCCTGG | |
| HPV82 | SD2- | 1435 | 1436 | CATCGGCAATGGACAGTGAAGCTGACCATACTCC | 2218 |
| | PVT1_002_exon3 | | | CTGGAGCCTTCTCCCGAGGTGCGCGGGTGACCT | |
| HPV82 | SD2- | 1437 | 1438 | CATCGGCAATGGACAGTGAAGTCTGAGCCTGATG | 2219 |
| | PVT1_004_exon1 | | | GATTTACAGTGATCTTCAGTGGTCTGGGG | |
| HPV82 | SD2- | 1439 | 1440 | CATCGGCAATGGACAGTGAAGCTCCGGGCAGAGC | 2220 |
| | PVT1_005_exon1 | | | GCGTGTGGCGGCCGAGCACATGGGCCCGCGGGCC | |
| | | | | GGGCGGGCTCGGGGCGGCCGGGACGAGGAGGGG | |
| | | | | CGACG | |

TABLE 2E

| Human | | Forward | | Reverse |
|---|---|---|---|---|
| gene | Forward primer | primer | Reverse primer | primer |
| name | Nucleic acid sequence | SEQ ID NO | Nucleic acid sequence | SEQ ID NO |
| ACTB | CCAGGTCATCACCATTGGCAAT | 1441 | CGTACAGGTCTTTGCGGATGT | 1442 |
| AKT1 | CCATGAGCGACGTGGCTATT | 1443 | CTCACGTTGGTCCACATCCT | 1444 |
| B2M | CTGTGCTCGCGCTACTCT | 1445 | CAACTTCAATGTCGGATGGATGAAAC | 1446 |
| BCL2 | GTGGATGACTGAGTACCTGAACC | 1447 | GGCCAAACTGAGCAGAGTCTT | 1448 |
| BRAF | CGGGACTCGAGTGATGATTGG | 1449 | CTGAGGTGTAGGTGCTGTCA | 1450 |
| CDH1 | CTCCTGAAAAGAGAGTGGAAGTGT | 1451 | CCGGATTAATCTCCAGCCAGTT | 1452 |
| CDKN2A | AACGCACCGAATAGTTACGGT | 1453 | ACGGGTCGGGTGAGAGT | 1454 |
| CDKN2B | CGGATCCCAACGGAGTCAA | 1455 | ACCGGTCGGGTGAGAGT | 1456 |
| ERBB2 | TCTTCCAGAACCTGCAAGTAATCC | 1457 | GGTGGGTGTTATGGTGGATGA | 1458 |
| FOS | AGGAGAATCCGAAGGGAAAGGAATA | 1459 | TCCTTCAGCAGGTTGGCAAT | 1460 |
| GAPDH | AGTCCACTGGCGTCTTCAC | 1461 | TGATCTTGAGGCTGTTGTCATACTTC | 1462 |
| GUSB | GCGAGTATGGAGCAGAAACGA | 1463 | AATTCCAAATGAGCTCTCCAACCA | 1464 |
| HRAS | CGGAATATAAGCTGGTGGTGGT | 1465 | GCACGTCTCCCCATCAATGA | 1466 |
| KRAS | GTGCAATGAGGGACCAGTACA | 1467 | CTACTAGGACCATAGGTACATCTTCAGA | 1468 |
| KRT10 | GATGAGCTGACCCTGACCAA | 1469 | GGCAGCATTCATTTCCACATTCAC | 1470 |
| KRT14 | AGGAGCTGGCCTACCTGAA | 1471 | CTTCTCATACTGGTCACGCATCT | 1472 |
| KRT17 | AACACTGAGCTGGAGGTGAAG | 1473 | CTGTAGCAGGATGTTGGCATTG | 1474 |
| MET | TGTGTGCATTCCCTATCAAATATGTCAA | 1475 | GCGCTTCACAGCCTGATGA | 1476 |
| MKI67 | CGTCGTGTCTCAAGATCTAGCTT | 1477 | TGAGTCATCTGCGGTACTGTCT | 1478 |
| MYC | GCTTCTCTGAAAGGCTCTCCTT | 1479 | AAATACGGCTGCACCGAGT | 1480 |
| NOTCH1 | CCGACGCACAAGGTGTCTT | 1481 | GTCGGCGTGTGAGTTGATGA | 1482 |
| PCNA | GACGGAGTGAAATTTTCTGCAAGT | 1483 | GAAGTTCAGGTACCTCAGTGCAAA | 1484 |
| PTEN | AGCGTGCAGATAATGACAAGGAA | 1485 | GATTTGACGGCTCCTCTACTGT | 1486 |
| RB1 | CGGTCTTCATGCAGAGACTGA | 1487 | GTGAAATATAGATGTTCCCTCCAGGAAT | 1488 |
| RPLP0 | GACGGATTACACCTTCCCACTT | 1489 | GACTCTTCCTTGGCTTCAACCTTA | 1490 |
| STAT1 | CGATGGGCTCAGCTTTCAGA | 1491 | ACAAAACCTCGTCCACGGAAT | 1492 |
| TERT | TCCTGCGTTTGGTGGATGAT | 1493 | CCTCGTCTTCTACAGGGAAGTTCA | 1494 |
| TOP2A | TGGGTGGTCCTGCAAAATCC | 1495 | ACATATTGATTTGGAGCCAGTTCTTCA | 1496 |
| TP53 | CTGGCCCCTGTCATCTTCTG | 1497 | CTTGGCCAGTTGGCAAAACAT | 1498 |
| WNT1 | CTGGAACTGTCCCACTGCT | 1499 | CAGGATTCGATGGAACCTTCTGA | 1500 |

TABLE 2Ebis

| | Forward | Reverse | | |
|---|---|---|---|---|
| Human | primer | primer | | Amplicon |
| gene | SEQ ID | SEQ ID | Amplicon | SEQ ID |
| name | NO | NO | nucleic acid sequence | NO |
| ACTB | 1441 | 1442 | GAGCGGTTCCGCTGCCCTGAGGCACTCTTCCAGCCTTCCTTCCTGGGCATGGA | 2221 |
| | | | GTCCTGTGGCATCCACGAAACTACCTTCAACTCCATCATGAAGTGTGACGTG | |
| | | | G | |
| AKT1 | 1443 | 1444 | GTGAAGGAGGGTTGGCTGCACAAACGAGGGGAGTACATCAAGACCTGGCGG | 2222 |
| | | | CCACGCTACTTCCTCCTCAAGAATGATGGCACCTTCATTGGCTACAAGGAGC | |
| | | | GGCCGC | |
| B2M | 1445 | 1446 | CTCTTTCTGGCCTGGAGGCTATCCAGCGTACTCCAAAGATTCAGGTTTACTCA | 2223 |
| | | | CGTCATCCAGCAGAGAATGGAAAGTCAAATTTCCTGAATTGCTATGTGTCTG | |
| | | | G | |
| BCL2 | 1447 | 1448 | GGCACCTGCACACCTGGATCCAGGATAACGGAGGCTGGGATGCCTTTGTGGA | 2224 |
| | | | ACTGTACGGCCCCAGCATGCGGCCTCTGTTTGATTTCTCCTGGCTGTCTCTG | |
| BRAF | 1449 | 1450 | GAGATTCCTGATGGGCAGATTACAGTGGGACAAAGAATTGGATCTGGATCAT | 2225 |
| | | | TTGGAACAGTCTACAAGGGAAAGTGGCATGGTGATGTGGCAGTGAAAATGTT | |
| | | | GAATG | |
| CDH1 | 1451 | 1452 | CCGAGGACTTTGGCGTGGGCCAGGAAATCACATCCTACACTGCCCAGGAGCC | 2226 |
| | | | AGACACATTTATGGAACAGAAAATAACATATCGGATTTGGAGAGACACTGCC | |
| CDKN2A | 1453 | 1454 | CGGAGGCCGATCCAGGTCATGATGATGGGCAGCGCCCGAGTGGCGGAGCTG | 2227 |
| | | | CTGCTGCTCCACGGCGCGGAGCCCAACTGCGCCGACCCCGCC | |
| CDKN2B | 1455 | 1456 | CCGTTTCGGGAGGCGCGCGATCCAGGTCATGATGATGGGCAGCGCCCGCGTG | 2228 |
| | | | GCGGAGCTGCTGCTGCTCCACGGCGCGGAGCCCAACTGCGCAGACCCTGCC | |
| ERBB2 | 1457 | 1458 | GGGGACGAATTCTGCACAATGGCGCCTACTCGCTGACCCTGCAAGGGCTGGG | 2229 |
| | | | CATCAGCTGGCTGGGGCTGCGCTCACTGAGGGAACTGGGCAGTGGACTGGCC | |
| | | | C | |
| FOS | 1459 | 1460 | AGATGGCTGCAGCCAAATGCCGCAACCGGAGGAGGGAGCTGACTGATACAC | 2230 |
| | | | TCCAAGCGGAGACAGACCAACTAGAAGATGAGAAGTCTGCTTTGCAGACCG | |
| | | | AG | |
| GAPDH | 1461 | 1462 | CACCATGGAGAAGGCTGGGGCTCATTTGCAGGGGGGAGCCAAAAGGGTCAT | 2231 |
| | | | CATCTCTGCCCCCTCTGCTGATGCCCCCATGTTCGTCATGGGTGTGAACCATG | |
| | | | A | |
| GUSB | 1463 | 1464 | TTGCAGGGTTTCACCAGGATCCACCTCTGATGTTCACTGAAGAGTACCAGAA | 2232 |
| | | | AAGTCTGCTAGAGCAGTACCATCTGGGTCTGGATCAAAAACGCAGAAAATAC | |
| | | | G | |
| HRAS | 1465 | 1466 | GGGCGCCGGCGGTGTGGGCAAGAGTGCGCTGACCATCCAGCTGATCCAGAA | 2233 |
| | | | CCATTTTGTGGACGAATACGACCCCACTATAGAGGATTCCTACCGGAAGCAG | |
| | | | GTGG | |
| KRAS | 1467 | 1468 | TGAGGACTGGGGAGGGCTTTCTTTGTGTATTTGCCATAAATAATACTAAATCA | 2234 |
| | | | TTTGAAGATATTCACCATTATAGAGAACAAATTAAAAGAGTTAAGGAC | |
| KRT10 | 1469 | 1470 | GGCTGACCTGGAGATGCAAATTGAGAGCCTGACTGAAGAGCTGGCCTATCTG | 2235 |
| | | | AAGAAGAACCACGAGGAGGAAATGAAAGACCTTCGAAATGTGTCCACTGGT | |
| | | | GAT | |
| KRT14 | 1471 | 1472 | GAAGAACCACGAGGAGGAGATGAACGCCCTGCGAGGCCAGGTGGGTGGTGA | 2236 |
| | | | GATCAATGTGGAGATGGACGCTGCCCCAGGCGTGGACCTGAGCCGCATCCTC | |
| | | | AACG | |
| KRT17 | 1473 | 1474 | ATCCGTGACTGGTACCAGAGGCAGGCCCCGGGGCCCGCCCGTGACTACAGCC | 2237 |
| | | | AGTACTACAGGACAATTGAGGAGCTGCAGAACAAGATCCTCACAGCCACCGT | |
| | | | GGA | |
| MET | 1475 | 1476 | CGACTTCTTCAACAAGATCGTCAACAAAAACAATGTGAGATGTCTCCAGCAT | 2238 |
| | | | TTTTACGGACCCAATCATGAGCACTGCTTTAATAGGACACTTCTGAGAAAT | |
| MKI67 | 1477 | 1478 | CTCTTCTGACCCTGATGAGAAAGCTCAAGATTCCAAGGCCTATTCAAAAATC | 2239 |
| | | | ACTGAAGGAAAAGTTTCAGGAAATCCTCAGGTACATATCAAGAATGTCAAAG | |
| | | | A | |
| MYC | 1479 | 1480 | GCAGCTGCTTAGACGCTGGATTTTTTTCGGGTAGTGGAAAACCAGCAGCCTC | 2240 |
| | | | CCGCGACGATGCCCCTCAACGTTAGCTTCACCAACAGGAACTATGACCTCGA | |
| | | | CTACG | |
| NOTCH1 | 1481 | 1482 | CCAGATCCTGATCCGGAACCGAGCCACAGACCTGGATGCCCGCATGCATGAT | 2241 |
| | | | GGCACGACGCCACTGATCCTGGCTGCCCGCCTGGCCGTGGAGGGCATGCTGG | |
| | | | AGGACC | |
| PCNA | 1483 | 1484 | GGAGAACTTGGAAATGGAAACATTAAATTGTCACAGACAAGTAATGTCGATA | 2242 |
| | | | AAGAGGAGGAAGCTGTTACCATAGAGATGAATGAACCAGTTCAACTAACT | |
| PTEN | 1485 | 1486 | TATCTAGTACTTACTTTAACAAAAAATGATCTTGACAAAGCAAATAAAGACA | 2243 |
| | | | AAGCCAACCGATACTTTTCTCCAAATTTTAAGGTGAAGCTGTACTTCACAAA | |
| | | | A | |
| RB1 | 1487 | 1488 | AAACAAATATTTTGCAGTATGCTTCCACCAGGCCCCCTACCTTGTCACCAATA | 2244 |
| | | | CCTCACATTCCTCGAAGCCCTTACAAGTTTCCTAGTTCACCCTTACGG | |
| RPLP0 | 1489 | 1490 | GCTGAAAAGGTCAAGGCCTTCTTGGCTGATCCATCTGCCTTTGTGGCTGCTGC | 2245 |
| | | | CCCTGTGGCTGCTGCCACCACAGCTGCTCCTGCTGCTGCTGCAGCCCCAGC | |
| STAT1 | 1491 | 1492 | AGTGCTGAGTTGGCAGTTTTCTTCTGTCACCAAAAGAGGTCTCAATGTGGACC | 2246 |
| | | | AGCTGAACATGTTGGGAGAGAAGCTTCTTGGTCCTAACGCCAGCCCCGATGG | |
| | | | TCTC | |
| TERT | 1493 | 1494 | TTCTTGTTGGTGACACCTCACCTCACCCACGCGAAAACCTTCCTCAGGACCCT | 2247 |
| | | | GGTCCGAGGTGTCCCTGAGTATGGCTGCGTGGTGAACTTGCGGAAGACAGTG | |
| | | | G | |
| TOP2A | 1495 | 1496 | CCAACTTTGATGTGCGTGAAATTGTAAATAACATCAGGCGTTTGATGGATGG | 2248 |
| | | | AGAAGAACCTTTGCCAATGCTTCCAAGTTACAAGAACTTCAAGGGTACTAT | |
| TP53 | 1497 | 1498 | TCCCTTCCCAGAAAACCTACCAGGGCAGCTACGGTTTCCGTCTGGGCTTCTTG | 2249 |
| | | | CATTCTGGGACAGCCAAGTCTGTGACTTGCACGTACTCCCCTGCCCTCAACAA | |
| | | | G | |
| WNT1 | 1499 | 1500 | CCAGGGCCCCACCTCTTCGGCAAGATCGTCAACCGAGGCTGTCGAGAAACGG | 2250 |
| | | | CGTTTATCTTCGCTATCACCTCCGCCGGGGTCACCCATTCGGTGGCGCGCTCC | |
| | | | TGC | |

Study Participants

Study participants were women aged from 25 to 65 years old referred for colposcopy consultation in French hospitals. The patients were referred for colposcopy in the context of a LSIL or a HSIL result at their cytology test performed in accordance with French recommendations regarding the cervical cancer screening program. Patients provided written informed consent according to French legislation.

Specimen Collection

Genital samples were collected just before performing colposcopy using a cervical sampling device, immersed and rinsed in a vial filled with 20mL of PreservCyt Solution (Hologic, USA), and sent at room temperature to the HPV National Reference Center (CNR) at Institut Pasteur, Paris, France. From July 2014 to April 2015, 84 patients were enrolled in the study, coming from 3 different French centers: CHU Angers (n=66); CHU Kremlin-Bicêtre (n=10); CHU Tours (n=6). Samples were removed of the study because of technical reasons (sample leakage, n=1) or legal issues (n=7) or because they were used for initial technical tests (RNA conservation, RNA extraction and amplification, n=4). The remaining 72 samples (HSIL=37; LSIL=35) were processed.

Data Collection

The following bio-clinical data were collected: date and results of the cytology test, age at the time of the cytology test, date and results of all available histological results posterior to colposcopy. As colposcopy was performed in the context of routine healthcare, biopsies were not performed in case of normal colposcopy.

HPV DNA Detection Using the PapilloCheck Test Kit (HPV DNA)

Upon reception at CNR, 16 mL of cytological sample were transferred into a 50 mL Falcon tube and centrifuged at 4,500 g for 10 minutes. The supernatant was removed and the pellet washed with 1 mL of PBS. Sample was then centrifuged again at 5000 g for 10 minutes and the supernatant removed. The pellet was frozen at −80° C. before DNA extraction. Following DNA extraction (Macherey Nagel, Germany), HPV detection was done using the PapilloCheck Test Kit (Greiner Bio-One GmbH, Germany) according to manufacturer instructions.

RNA Extraction and Characterization

In parallel to the HPV DNA procedure, 3×1 mL aliquots of cytological specimen were centrifuged at 14,000 rpm for 7 minutes, the supernatant was removed and the pellet was washed with 1 mL of PBS. Sample was then centrifuged again at 14,000 rpm for 7 minutes and the supernatant removed. The pellet was frozen at −80° C. before RNA extraction. RNA extractions were done using the PicoPure RNA Isolation kit (Thermo Fisher Scientific,), including on-column DNAse treatment, with a final elution volume of 30 μl. Total RNA was quantified on a Nanodrop (Life Technologies) and RNA integrity was evaluated on a Bioanalyzer RNA 6000 pico chip (Agilent) using the RIN (RNA Integrity Number), a quality score ranging from 1 (strongly degraded RNA) to 10 (intact RNA). For each sample, RT-qPCR targeting mRNA from on housekeeping genes ACTB (forward primer: CATCGAGCACGGCATCGTCA (SEQ ID NO: 2258); reverse primer: TAGCACAGCCTGGATAGCAAC (SEQ ID NO: 2259); amplicon size=210 bp) and GAPDH (forward primer: GAAGGTGAAGGTCGGAGTC (SEQ ID NO: 2260); reverse primer: GAAGATGGTGATGGGATTTC (SEQ ID NO: 2261); amplicon size=226 bp) were done in a SYBR Green format with 45 cycles of amplification. RT-negative (RT-) PCR were also run to evaluate the presence of residual DNA after RNA extraction.

Amplification and Sequencing

Starting from RNA, cDNA were generated using the SuperScript III (n=17 samples) or Superscript IV (n=55 samples) (Thermo Fisher Scientific) with random hexamers and a final RNAse H treatment. Libraries were prepared using the Ion AmpliSeq Library Kit 2.0 and AmpliSeq custom panel WG_WG00141, with 21 cycles of amplification before adapter's ligation. Each sample was barcoded individually. Only positive libraries were sequenced. In total, 55 clinical samples plus 1 cellular model (SiHa) were sequenced on 4 Ion Proton runs.

Sequencing Data Processing

Reads were aligned to the reference sequences of the amplicons using STAR23 v2.5.3a in local alignment mode (parameter -alignEndsType EndToEnd), by only reporting uniquely mapped reads (-outFilterMultimapNmax 1) and turning off splicing alignment (-alignIntronMax 1). The expression of each amplicon was evaluated by the number of sequencing reads uniquely mapping to their respective sequence (read counts). For reference sequences containing a splice junction, only reads mapping at the junction site and encompassing at least 10 bases before and 10 bases after the junction were kept.

HSIL Prediction Model

Selection of Amplicons

Read counts were normalized by the size of the library (each read count was divided by a ratio of the library size for a given sample to that of the average library size across samples) and the 215 amplicons capturing splice junctions (sp) of the 16 high-risk or putative high risk HPV were selected. These amplicons have been annotated with generic names with respect to the type of transcripts they capture, which are shared across HPV species (e.g. “SD1-SA1”, see FIG. 2). Amplicons capturing homolog generic splice junctions conserved across the 16 HPV species were summed up, leading to the definition of 18 variables used as predictors in the model. 33 out of the 55 clinical samples have been selected as presenting enough coverage of these specific amplicons (20 mono-infected and 13 multi-infected samples). The remaining 22 samples of the dataset were not used in the logistic regression analysis because they had missing or too low expression signal at splice junctions for the prediction, reflecting for example HPV-negative samples.

Logistic Regression Model

Calling high grade cytology Y as taking the value 1 for high grade (HSIL) and 0 for low grade (LSIL), and a set of amplicons x, a logistic regression model was used to predict the probability that a given observation belongs to the “1” class versus the probability that it belongs to the “0” class. Logistic regression models the log odds of the event (here the grade of the cytology) as a function of the predictor variables (here the amplicon expression estimated by its read count). Formally, the logistic regression model assumes that the log odds is a linear function of the predictors:

$$( π ) = ln ( \frac{π}{1 - π} ) = {β}_{0} + {β}^{t} x$$

where indicates the probability of the event (being of high-grade), βi are the regression coefficients, and xi the explanatory variables, in our case the log 2 number of reads mapping to the amplicons.

Solving for π, this gives:

$$π = \frac{1}{1 + {e}^{- ( {β}_{0} + β x )}}$$

Implementation of the Logistic Regression Model

To limit overfitting, the inventors used L2-norm (ridge) regularization, which allows shrinking the magnitudes of the regression coefficients such that they will better fit future data. The inventors estimated the logistic model using the R (http://www.r-project.org/) package glmnet (Friedman J, Hastie T, Tibshirani R. Regularization Paths for Generalized Linear Models via Coordinate Descent. J Stat Softw, 2010, 33:1-22). Leave-one-out (LOO) cross-validation was used to pick the regularization parameter λ, the one that gives minimum mean cross-validated misclassification error was used. Using λ as the regularization parameter, the model output consisted in an estimate of a coefficient value β for each variable in the logistic regression model. This model was then used to predict the grade of the multi-infected observations, by treating each HPV species separately.

Training Set and Test Set

The model was built upon the clinical outcome LSIL or HSIL obtained from the cytological analysis, and estimated on a training set consisting of 20 mono-infected samples (5 LSIL and 15 HSIL) in order to avoid a confusion bias. It is indeed anticipated that, in the case of multi-infected samples, several HPV could contribute differently to the progression of the lesion or to a mix of several grades within the same sample, because they are engaged in different stages of their cycle. The performance of the model was then evaluated on a test set consisting of 13 multi-infected samples. In this case, the set of amplicons of each HPV species was used separately to classify the multi-infected samples, to get one prediction per HPV, as done for the mono-infected samples. For example if a sample had expression of amplicons from both HPV16 and HPV32, two predictions were given: one using only sequencing reads mapping to HPV16, and one using only sequencing reads mapping to HPV32. Like this it became possible to interpret the results finely from a virological point of view, as the inventors could discriminate which HPV was responsible of the lesion.

Results:

Evaluation of Transport Medium for RNA Conservation

The stability of total RNA from cervical cells at room temperature was evaluated in four solutions: PreservCyt (Hologic), the most widely used solution for gynecological specimen collection; NovaPrep HQ+ Solution (Novaprep), a competitor product used for cells and DNA recovery but never evaluated for RNA conservation; RNA Protect Cell Reagent (Qiagen), a popular solution for RNA stability; and NucliSens Lysis Buffer (BioMérieux), a lysis buffer part of the NucliSens automated acid nucleic procedure which has been described as a RNA stabilizer. The amount of spiked HPV16-positive cervical squamous cell carcinoma cells (SiHa) was calibrated to be representative of a cervical smear. After 48 h at room temperature, RT-qPCR measurement of cellular and viral transcripts showed no or little RNA loss in PreservCyt, only limited RNA degradation (<1 log) in RNA Protect and NucliSens Lysis Buffer, and a marked RNA loss in NovaPrep HQ+ Solution (>2 log). After 7 days and up to 21 days, only the PreservCyt solution provided RNA quality with a limited RNA degradation pattern as indicated by the detection of 18S and 28S rRNA. The inventors therefore decided to use the PreservCyt solution to collect the gynecological specimen of the study.

HPV RNA-Seq AmpliSeq Custom Panel

Transcriptomic maps known for HPV1620 and HPV1821 were used to predict unknown but likely splice donor and splice acceptor sites for HPV31, 33, 35, 39, 45, 51, 52, 56, 58, 59, 66, 68, 73, and 82 (FIGS. 1 and 2). The resulting reconstructed transcripts, as well as HPV genomic sequences, were used as a template for the design of an Ion AmpliSeq panel targeting 16 high-risk or putative high-risk HPV and named HPV RNA-Seq. Putative breakpoints in HPV genomes, and 30 human cellular genes used as internal controls, were also added to the design. In total, 750 sequences are targeted by a single mix made of 525 unique primers (Table 1 and Tableau 2A-2E).

Samples, RNA & Sequencing

72 gynecological samples (HSIL=37; LSIL=35) coming from 3 different French centers (Angers, Kremlin-Bicêtre and Tours) and collected in PreservCyt solution were processed with RNA extraction using a method designed to recover total RNA from as little as a single cell (PicoPure RNA Isolation kit, Thermo Fisher Scientific, USA). In most of the cases total RNA was measurable using a Nanodrop (70/72 positive, average on positive RNA eluates=18 ng/μL) and was detectable on a Bioanalyzer pico RNA chip with a pattern indicating RNA degradation (63/72 positive, average RNA Integrity Number on positive=2.2). RT-qPCR performed for all samples on ACTB mRNA (amplicon size=210 bp) and GAPDH mRNA (amplicon size=226 bp) indicated that RNA quality was compatible with amplification of 200-250 bp size fragments (ACTB mRNA average Ct=27.8; GAPDH mRNA average Ct=30.1). Samples that failed passing this initial RT-PCR quality control were not sequenced. qPCR performed after omitting the reverse transcription step (RT-) were also run and showed in general no or little traces of residual genomic DNA (ACTB DNA average Ct=38.4; GAPDH DNA average Ct=35.6). Note, the presence of residual cellular DNA or HPV DNA in RNA preparation is not a major concern since the AmpliSeq assay can differentiate between HPV transcripts and genomic sequences. AmpliSeq libraries were initiated from total RNA and were positive after 21 cycles of amplification for 55 samples (i.e. detectable on a Bioanalyzer HS DNA chip). Attempts to add one or two amplification cycles did not bring any significant improvement to the results (data not shown). In total, 55 patients (HSIL=27; LSIL=28), plus SiHa HPV16-positive cells as a control, had been sequenced on Ion Proton. The sequencing reads were aligned to the target sequences and read counts were generated. An average of 2.4 million usable reads per sample was reached (min=0.02M; max=8.3M), among which an average of 2.1 million reads mapped to the human sequences (hg) used as internal controls (min=0.01M; max=8.06M). The detection of highly expressed human sequences in all samples, even though inter-sample variations were observed, contributed to validate the sequencing procedure, which is important especially for the interpretation of HPV-negative samples. Rare non-zero values were also observed for some of the numerous HPV-human fusion sequences (fus) that were hypothesized but were all false positives, identified as such because only half of the reference sequences were covered by reads.

HPV RNA-Seq Used for HPV Detection and Genotyping

The first application of HPV RNA-Seq is to detect the presence in a given sample of any of the 16 high-risk or putative high-risk HPV targeted by the panel. The number of reads mapping to HPV-specific amplicons (i.e. the sum of categories “sp”, “unsp” and “gen”) was used to detect the presence of a given HPV genotype. To help determining a threshold for detection, we took as a reference a HPV DNA test validated for clinical use (PapilloCheck, Greiner Bio-One GmbH). The best sensitivity and specificity values between the two tests were obtained for threshold of 100-200 reads (FIG. 3). For example, a threshold value of 150 reads resulted in a Sensitivity (Se(HPV-DNA)) of 97.3%, a Specificity (Sp(HPV-DNA)) of 83.3%, leading to a Positive Predictive Value (PPV(HPV-DNA)) of 92.3% and a Negative Predictive Value (NPV(HPV-DNA)) of 93.8% for detecting high-risk HPV in this population composed of around 50% of HSIL and 50% of LSIL (Table 3). Table 3 shows the performances of HPV RNA-Seq for HPV detection vs HPV DNA (PapilloCheck) at threshold value of 150 reads. Sensitivity (Se), Specificity (Sp), Positive Predictive Value (PPV) and Negative Predictive Value (NPV) are given. HPV+ means that at least one HPV genotype is identified in a patient.

TABLE 3

| | | HPV DNA | | | |
|---|---|---|---|---|---|
| | | HPV+ | HPV− | Se(HPV- | 97.3% |
| | | | | DNA) | |
| HPV RNA- | HPV+ | 36 | 3 | Sp(HPV- | 83.3% |
| Seq | | | | DNA) | |
| | HPV− | 1 | 15 | PPV(HPV- | 92.3% |
| | | | | DNA) | |
| | | | | NPV(HPV- | 93.8% |
| | | | | DNA) | |

A more detailed view of the genotypes identified by both techniques is given in FIG. 4. The number of mono-infected, multi-infected, or HPV-negative samples identified by the two tests is summarized in Table 4. Note that, because the HPV DNA test can detect the 16 high-risk or putative high-risk HPV captured by HPV RNA-Seq (HPV16, 18, 31, 33, 35, 39, 45, 51, 52, 56, 58, 59, 66, 68, 73, and 82) plus 8 additional low-risk HPV (HPV6, 11, 40, 42, 43, 44/45, 53 and 70), the comparison was based only on the 16 HPV common to both tests.

TABLE 4

| | HPV RNA- | |
|---|---|---|
| | Seq | HPV DNA |
| Mono-infected samples | 26 | 27 |
| Multi-infected samples | 13 | 10 |
| HPV-negative samples | 16 | 18 |

Using a threshold value of 150 reads, HPV RNA-Seq detected two more positive patients than the HPV DNA test (n=39 vs n=37, Table 3). HPV RNA-Seq identified the presence of more than one HPV for three more patients than the HPV DNA test (n=13 vs n=10 multi-infected samples, Table 4). Globally, HPV16 was found at a slightly weaker occurrence by HPV RNA-Seq (n=18 vs n=19) in favor of other genotypes such as HPV31, 33, 45, 52, 56, 58 or 66 which were less commonly found by the HPV DNA test (HPV31 n=5 vs n=4; HPV33 n=3 vs n=1; HPV45 n=3 vs n=2; HPV52 n=5 vs n=3; HPV56 n=4 vs n=2; HPV58 n=5 vs n=4; HPV66 n=2 vs n=1, FIG. 4). Apart from HPV16, only HPV51 was less frequently found by HPV RNA-Seq than by HPV DNA (n=2 vs n=3). The cellular model (SiHa) gave only HPV16 signal in both tests, as expected.

HPV RNA-Seq Used as a Marker of High-Grade Cytology

The inventors conducted an exploratory analysis on 20 of the mono-infected samples in which they showed that HPV RNA splice junctions could be used to predict high-grade cytology. They focused the analysis on amplicons capturing splice junctions (category “sp”) to be sure to detect HPV transcripts. However, the number of mono-infected samples (n=20) used as training set was small, in particular the number of samples of LSIL (n=5). LOO cross-validation was used to pick the lambda giving the minimum cross-validated error using ridge regularization. Lambda=0.08 gave a mean cross-validated error of 15%. The inventors also computed a 20% prediction error using nested cross-validation. This error rate can be seen as an indicator of how the model could fit future datasets. The inventors used the corresponding parameter to fit a regularized logistic regression model, assigning a coefficient to each amplicon (Table 5) and a probability of being of high-grade to each sample (Table 6). In table 5, the first and fourth columns give the id of the splice junction captured by the amplicon, the second column gives the coefficient assigned by the logistic regression, the third column indicate whether the splice junction comes from a “late” or “early” transcript.

TABLE 5

| junction | Coefficient | name_transcript_category | name_transcript_contents |
|---|---|---|---|
| (Intercept) | 0.468298365 | | |
| SD2_SA10 | −0.693322203 | Late | L1 |
| SD3_SA4 | 0.545728771 | Early | (E1) E4 E5 |
| SD1_SA4 | 0.387642812 | Early | (E6) E2 E5 |
| SD2_SA4 | −0.262522618 | Early | (E7) E2 E5 |
| SD1_SA2 | 0.146954179 | Early | E6 E7 |
| SD2_SA5 | 0.12050536 | Early | (E7) E2 E5 |
| SD1_SA6 | 0.107204358 | Early | (E6) E4 E5 |
| SD5_SA10 | 0.096088118 | Late | L1 |
| SD3_SA6 | 0.093052957 | Early | (E1) E4 E5 |
| SD1_SA5 | 0.092877361 | Early | (E6) E2 E5 |
| SD2_SA6 | −0.088655106 | Early | (E7) E4 E5 |
| SD1_SA1 | 0.07669912 | Early | E6 E7 |
| SD1_SA3 | 0.069688722 | Early | E6 E7 |
| SD2_SA8 | 0.061867993 | Early | (E7) E4 E5 |
| SD3_SA5 | 0.051702326 | Early | (E1) E4 E5 |
| SD2_SA9 | −0.040972141 | Late | L1 |
| SD5_SA9 | −0.026083777 | Late | L1 |
| SD3_SA8 | 0 | Early | (E1) E4 E5 |

TABLE 6

| sample | prediction_score | prediction_class | prediction_class | prediction_accuracy |
|---|---|---|---|---|
| IonXpress_039_115 | 0.115 | −1 | LSIL | TRUE |
| IonXpress_033_730 | 0.204 | −1 | LSIL | TRUE |
| IonXpress_038_114 | 0.259 | −1 | LSIL | TRUE |
| 1492 | 0.425 | −1 | LSIL | TRUE |
| IonXpress_019_2613 | 0.562 | 1 | LSIL | FALSE |
| IonXpress_027_598 | 0.653 | 1 | HSIL | TRUE |
| 729 | 0.716 | 1 | HSIL | TRUE |
| 567 | 0.718 | 1 | HSIL | TRUE |
| IonXpress_018_2439 | 0.902 | 1 | HSIL | TRUE |
| 610 | 0.904 | 1 | HSIL | TRUE |
| 1066 | 0.911 | 1 | HSIL | TRUE |
| IonXpress_034_758 | 0.919 | 1 | HSIL | TRUE |
| 1122 | 0.934 | 1 | HSIL | TRUE |
| 25 | 0.944 | 1 | HSIL | TRUE |
| IonXpress_037_1267 | 0.947 | 1 | HSIL | TRUE |
| IonXpress_024_26 | 0.965 | 1 | HSIL | TRUE |
| IonXpress_025_538 | 0.97 | 1 | HSIL | TRUE |
| 752 | 0.976 | 1 | HSIL | TRUE |
| IonXpress_021_443 | 0.984 | 1 | HSIL | TRUE |
| 2612 | 0.993 | 1 | HSIL | TRUE |

Table 6 shows the classification results of the (ridge) logistic regression. The first column gives the sample id, the second column gives the probability estimate that the sample is HSIL, the third and fourth columns gives the corresponding prediction, the fifth column contains TRUE if the prediction is consistent with the grade evaluated by cytology.

The grade of the 20 mono-infected samples was classified correctly, except for one observation (Table 5). It is interesting to note that this unique misclassified sample (IonXpress_019_2613), which was classified LSIL by the cytological analysis, was further found as containing a mixture of LSIL and HSIL lesions after histological examination performed more than one year after the sampling done for HPV RNA-Seq/cytology sampling.

The estimated model was then used to classify the 13 multi-infected samples, with each HPV species present within one sample being classified individually for its implication in HSIL development. If at least one HPV species gave a HSIL prediction, the sample was considered to be HSIL. We calculated performances for HSIL prediction for all samples, considering as not being of high-grade both the six samples without sufficient coverage of the splice junctions and the 16 HPV-negative samples not exceeding the threshold of HPV detection. The calculated performances for HSIL prediction in comparison to cytology for the 55 patients (mono-infected, multi-infected and HPV-negative) were Se(cyto)=66.7%, Sp(cyto)=85.7%, PPV(cyto)=81.8% and NPV(cyto)=72.7% (Table 7A). The performances were also calculated for the subset of 39 samples having at least one HPV identified by HPV RNA-Seq, giving in this case Se(cyto/HR+)=94.7%, Sp(cyto/HR+)=80.0%, PPV(cyto/HR+)=81.8% and NPV(cyto/HR+)=94.1% (Table 7B). In table 7, Sensitivity (Se), Specificity (Sp), Positive Predictive Value (PPV) and Negative Predictive Value (NPV) are given. “Not HSIL” means that either no HPV was detected in the sample by HPV RNA-Seq or that none of the HPV genotypes detected were given HSIL prediction.

TABLE 7

| A | | Cytology | | | |
|---|---|---|---|---|---|
| | | HSIL | LSIL | Se(cyto) | 66.7% |
| HPV RNA- | HSIL | 18 | 4 | Sp(cyto) | 85.7% |
| Seq | | | | | |
| | Not | 9 | 24 | PPV(cyto) | 81.8% |
| | HSIL | | | | |
| | | | | NPV(cyto) | 72.7% |
| B | | Cytology | | | |
| | | HSIL | LSIL | Se(cyto/HR+) | 94.7% |
| HPV RNA- | HSIL | 18 | 4 | Sp(cyto/HR+) | 80.0% |
| Seq HR+ | | | | | |
| | Not | 1 | 16 | PPV(cyto/HR+) | 81.8% |
| | HSIL | | | | |
| | | | | NPV(cyto/HR+) | 94.1% |

Note that the ratio HSIL to LSIL remained similar between these two populations (around 1:1), making the comparison of the PPV and the NPV possible. Finally a summary of the results for HPV detection and genotyping (HPV RNA-Seq vs HPV DNA) and high-grade cytology prediction (HPV RNA-Seq vs cytology), including posterior histological data of cervix biopsies when available, is presented in Table 8.

TABLE 8

| | | HPV RNA-Seq | | | | | | | |
|---|---|---|---|---|---|---|---|---|---|
| | | | Marker of HSIL | | | | | | |
| | | Genotyping | Per HPV | | | | | | |
| | | Per patient | Not enough coverage | Not | | Per patient | | | Time (days) |
| Sample name | HPV DNA | Detection | on splice junctions | HSIL | HSIL | Prediction | Cytology | Histology | cyto-histo |
| D-15-0041_1066_BC13 | 16 | 16 | | | 16 | HSIL | HSIL | HSIL | 55 |
| D-15-0041_1122_BC14 | 16 | 16 | | | 16 | HSIL | HSIL | HSIL | 130 |
| D-15-0041_1124_BC5 | 16, 39 | 16, 39 | 39 | 16 | | Not HSIL | LSIL | HSIL | [70-434] |
| D-15-0041_1490_BC6 | 16, 39 | 16, 35, 39 | | 39 | 16, 35 | HSIL | LSIL | HSIL | 67 |
| D-15-0041_1492_BC7 | 16 | 16 | | 16 | | Not HSIL | LSIL | LSIL | 81 |
| D-15-0041_151_BC15 | 16, (53) | 16 | | | 16 | HSIL | LSIL | HSIL | 130 |
| D-15-0041_152_BC16 | 16, (42) | 16, 52, 82 | 16, 52, 82 | | | Not HSIL | LSIL | LSIL | 41 |
| D-15-0041_2209_BC11 | 16, (42), 52 | 16, 39, 52 | 39 | 16, 52 | | Not HSIL | LSIL | HSIL | n.d. |
| D-15-0041_250_BC12 | 16, 39, (42) | 16, 39 | | 16, 39 | | Not HSIL | LSIL | LSIL | 55 |
| D-15-0041_25_BC4 | 16 | 16 | | | 16 | HSIL | HSIL | HSIL | 75 |
| D-15-0041_2612_BC8 | 16 | 16 | | | 16 | HSIL | HSIL | n.d. | n.d. |
| D-15-0041_567_BC9 | 16 | 16 | | | 16 | HSIL | HSIL | HSIL | n.d. |
| D-15-0041_610_BC2 | 16 | 16 | | | 16 | HSIL | HSIL | HSIL | 113 |
| D-15-0041_729_BC3 | 16 | 16 | | | 16 | HSIL | HSIL | HSIL | 59 |
| D-15-0041_752_BC10 | 16 | 16 | | | 16 | HSIL | HSIL | HSIL | 444 |
| IonXpress_017_2437 | (43), 51 | 51 | | 51 | | Not HSIL | LSIL | LSIL | 195 |
| IonXpress_017_251 | neg | neg | | | | Not HSIL | HSIL | LSIL | 85 |
| IonXpress_018_2439 | 58 | 58 | | | 58 | HSIL | HSIL | LSIL | 164 |
| IonXpress_018_440 | neg | neg | | | | Not HSIL | LSIL | LSIL | 38 |
| IonXpress_019_2613 | 16 | 16 | | | 16 | HSIL | LSIL | HSIL | [416-780] |
| IonXpress_020_3137 | (53) | 56 | 56 | | | Not HSIL | HSIL | HSIL | 350 |
| IonXpress_021_10 | 56, (44/55) | 56 | | 56 | | Not HSIL | LSIL | n.d. | 130 |
| IonXpress_021_443 | 58 | 33, 58 | 33 | | 58 | HSIL | HSIL | LSIL | 99 |
| IonXpress_022_23 | neg | neg | | | | Not HSIL | HSIL | HSIL | n.d. |
| IonXpress_022_444 | 16, 33 | 16, 33 | | 33 | 16 | HSIL | HSIL | HSIL | 69 |
| IonXpress_023_24 | (6), (11), (53) | neg | | | | Not HSIL | HSIL | HSIL | [0-13] |
| IonXpress_023_536 | neg | neg | | | | Not HSIL | LSIL | LSIL | 101 |
| IonXpress_024_26 | 45 | 45 | | | 45 | HSIL | HSIL | HSIL | 106 |
| IonXpress_024_537 | neg | neg | | | | Not HSIL | LSIL | LSIL | 71 |
| IonXpress_025_457 | neg | neg | | | | Not HSIL | LSIL | LSIL | 278 |
| IonXpress_025_538 | 35 | 31, 35 | 31 | | 35 | HSIL | HSIL | HSIL | 191 |
| IonXpress_026_539 | neg | neg | | | | Not HSIL | LSIL | n.d. | n.d. |
| IonXpress_026_565 | 16 | neg | | | | Not HSIL | HSIL | HSIL | 65 |
| IonXpress_027_598 | 31 | 31 | | | 31 | HSIL | HSIL | HSIL | 52 |
| IonXpress_028_609 | 35, 52 | 52 | | | 52 | HSIL | LSIL | HSIL | 83 |
| IonXpress_029_611 | neg | neg | | | | Not HSIL | HSIL | n.d. | n.d. |
| IonXpress_030_612 | neg | neg | | | | Not HSIL | LSIL | LSIL | 113 |
| IonXpress_031_613 | 35, 39, (44/55) | 35, 39 | | 35, 39 | | Not HSIL | LSIL | LSIL | 83 |
| IonXpress_032_728 | neg | neg | | | | Not HSIL | HSIL | HSIL | 59 |
| IonXpress_033_730 | 31 | 31 | | 31 | | Not HSIL | LSIL | HSIL | [211-575] |
| IonXpress_034_758 | 58 | 58 | | | 58 | HSIL | HSIL | HSIL | 43 |
| IonXpress_035_1150 | 16, 39, 52 | 16, 39, 52 | | 52 | 16, 39 | HSIL | HSIL | HSIL | 125 |
| IonXpress_036_1151 | (11), 31 | 31 | | | 31 | HSIL | HSIL | HSIL | 125 |
| IonXpress_036_98 | (42) | neg | | | | Not HSIL | LSIL | n.d. | 20 |
| IonXpress_037_100 | neg | neg | | | | Not HSIL | LSIL | LSIL | 57 |
| IonXpress_037_1267 | 45 | 45 | | | 45 | HSIL | HSIL | LSIL | 71 |
| IonXpress_038_114 | 31 | 31 | | 31 | | Not HSIL | LSIL | HSIL | 154 |
| IonXpress_038_1597 | neg | neg | | | | Not HSIL | HSIL | HSIL | 85 |
| IonXpress_039_115 | 56 | 56 | | 56 | | Not HSIL | LSIL | LSIL | 34 |
| IonXpress_039_1598 | neg | neg | | | | Not HSIL | HSIL | LSIL | 115 |
| IonXpress_041_1650 | 66, (70) | 56, 66 | 56, 66 | | | Not HSIL | LSIL | LSIL | 115 |
| IonXpress_043_1871 | 51, 58, 68, 73 | 33, 51, 58, 68 | 33 | 51, 58, 68 | | Not HSIL | LSIL | LSIL | 101 |
| IonXpress_044_2064 | 39, 51 | 45 | 45 | | | Not HSIL | LSIL | HSIL | 129 |
| IonXpress_045_2065 | neg | 52, 58 | 52, 58 | | | Not HSIL | LSIL | LSIL | 160 |
| IonXpress_046_2066 | (6) | 66 | 66 | | | Not HSIL | LSIL | HSIL | 99 |

HPV RNA-Seq Used as a Triage Test

The performances of HPV RNA-Seq as a triage test were evaluated using histology as gold standard. Results from histological examination were, however, not available for all patients. The time interval separating HPV RNA-Seq/cytology tests from histological analysis, varying between 0 and 780 days, was another limitation in this study. To try to overcome these drawbacks, we compared the performances of HPV RNA-Seq vs histology to the performances of cytology vs histology, considering either all available samples or only samples for which histology was done less than 3 months after HPV RNA-Seq/cytology or only samples for which histology was done less than 6 months after HPV RNA-Seq/cytology. In addition and for each category, we made the distinction between the performances obtained when HPV RNA-Seq HPV-positive and HPV-negative patients were grouped together or when only HPV-positive patients were considered. Calculation of the PPV as a function of HSIL prevalence in the population was also done.

### REFERENCES

Throughout this application, various references describe the state of the art to which this invention pertains. The disclosures of these references are hereby incorporated by reference into the present application.

1-15. (canceled)

16. An in vitro method for detecting HSIL in a biological sample comprising the steps of:
(a) extraction of RNA from the biological sample,
(b) reverse transcription of the RNA so as to generate cDNA,
(c) amplification of the cDNA generated at step (b) with a composition of primers so as to produce amplicon(s),
(d) quantifying the expression level of each amplicon produced at step (c),
(e) determining if the biological sample comprises HSIL based on the expression level of the amplicons quantified at step (d),
wherein the composition of primers comprises a first set of pairs of primers, called splice junctions set of primers, which comprises:
at least 2 pairs of primers of a first subset of HPV16 specific pairs of primers able to produce the amplicons having the nucleic acid sequence selected from the group consisting of SEQ ID NO: 1501 to SEQ ID NO: 1514; and
at least 2 pairs of primers of a second subset of HPV18 specific pairs of primers able to produce the amplicons having the nucleic acid sequence selected from the group consisting of SEQ ID NO: 1515 to SEQ ID NO: 1532; and
at least 2 pairs of primers of a third subset of HPV31 specific pairs of primers able to produce the amplicons having the nucleic acid sequence selected from the group consisting of SEQ ID NO: 1533 to SEQ ID NO: 1546; and
at least 2 pairs of primers of a fourth subset of HPV33 specific pairs of primers able to produce the amplicons having the nucleic acid sequence selected from the group consisting of SEQ ID NO:1547 to SEQ ID NO:1559; and
at least 2 pairs of primers of a fifth subset of HPV35 specific pairs of primers able to produce the amplicons having the nucleic acid sequence selected from the group consisting of SEQ ID NO: 1560 to SEQ ID NO: 1573; and
at least 2 pairs of primers of a sixth subset of HPV39 specific pairs of primers able to produce the amplicons having the nucleic acid sequence selected from the group consisting of SEQ ID NO: 1574 to SEQ ID NO: 1583; and
at least 2 pairs of primers of a seventh subset of HPV45 specific pairs of primers able to produce the amplicons having the nucleic acid sequence selected from the group consisting of SEQ ID NO: 1584 to SEQ ID NO: 1597; and
at least 2 pairs of primers of a eighth subset HPV51 specific of pairs of primers able to produce the amplicons having the nucleic acid sequence selected from the group consisting of SEQ ID NO: 1598 to SEQ ID NO: 1607; and
at least 2 pairs of primers of a ninth subset of HPV52 specific pairs of primers able to produce the amplicons having the nucleic acid sequence selected from the group consisting of SEQ ID NO: 1608 to SEQ ID NO: 1623; and
at least 2 pairs of primers of a tenth subset EIPV56 specific of pairs of primers able to produce the amplicons having the nucleic acid sequence selected from the group consisting of SEQ ID NO: 1624 to SEQ ID NO: 1639; and
at least 2 pairs of primers of an eleventh subset HPV58 specific of pairs of primers able to produce the amplicons having the nucleic acid sequence selected from the group consisting of SEQ ID NO:1640 to SEQ ID NO:1652; and
at least 2 pairs of primers of a twelfth subset of HPV59 specific pairs of primers able to produce the amplicons having the nucleic acid sequence selected from the group consisting of SEQ ID NO: 1653 to SEQ ID NO: 1666; and
at least 2 pairs of primers of a thirteenth subset of HPV66 specific pairs of primers able to produce the amplicons having the nucleic acid sequence selected from the group consisting of SEQ ID NO: 1667 to SEQ ID NO: 1681, and optionally:
at least 2 pairs of primers of a fourteenth subset of HPV68 specific pairs of primers able to produce the amplicons having the nucleic acid sequence selected from the group consisting of SEQ ID NO: 1682 to SEQ ID NO: 1691; and/or
at least 2 pairs of primers of a fifteenth subset of HPV73 specific pairs of primers able to produce the amplicons having the nucleic acid sequence selected from the group consisting of SEQ ID NO: 1692 to SEQ ID NO: 1704; and/or
at least 2 pairs of primers of a sixteenth subset of HPV82 specific pairs of primers able to produce the amplicons having the nucleic acid sequence selected from the group consisting of SEQ ID NO: 1705 to SEQ ID NO: 1715.

17. The in vitro method for detecting HSIL according to claim 16, wherein the splice junctions set of primers comprises at least 10 pairs of primers of the first to the thirteenth subsets of pairs of primers and optionally at least 10 pairs of primers of the fourteenth and/or the fifteenth and/or the sixteenth subsets of pairs of primers.

18. The in vitro method for detecting HSIL according to claim 16, wherein the composition of primers comprises an additional set of primers selected from the group consisting of:
a second set of primers, called unsplice junctions set of primers wherein the primers target high risk and optionally putative high risk HPV genomic regions spanning either splice donor or splice acceptor sites in the absence of any splice event,
a third set of primers, called genomic set of primers wherein the primers target high risk and optionally putative high risk HPV genomic regions away from any splice donor or splice acceptor sites, and
a fourth set of primers, called fusion set of primers wherein the primers target high risk and optionally putative high risk HPV fusion transcripts.

19. The in vitro method for detecting HSIL according to claim 16, wherein the step of determining if the biological sample comprises HSIL comprises:
for each type of HPV selected from the group consisting of HPV16, HPV18, HPV31, HPV33, HPV35, HPV39, HPV45, HPV51, HPV52, HPV56, HPV58, HPV59, and HPV66 and optionally HPV68 and/or HPV73 and/or HPV82, a step calculating a probability p_{HPVj} that the biological sample comprises an HSIL of HPVⱼ type wherein ⱼ=16, 18, 31, 33, 35, 39, 45, 51, 52, 56, 58, 59, 66, 68, 73 and/or 82 using the following formula:
$$logit ( {p}_{H P V j} ) = {β}_{0} + {\sum }_{i = 1}^{25} ( {β}_{i} {X}_{i j} )$$
with:
β₀ is the intercept,
βᵢ is a coefficient corresponding to a given splice junction, called splice junction i,
Xᵢⱼ is a variable depending on the quantified level of expression of the amplicon corresponding to the splice junction i for the HPVⱼ,
wherein if one p_{HPVj} is higher than 0.5, it is indicative of the presence of an HPVⱼ HSIL in the biological sample.

20. The in vitro method for detecting HSIL according to claim 19, wherein:
the amplicons corresponding to the splice junction i=1 are respectively SEQ ID NO: 1501 for HPV16, SEQ ID NO: 1515 for HPV18, SEQ ID NO: 1533 for HPV31, SEQ ID NO: 1547 for HPV33, SEQ ID NO: 1560 for HPV35, SEQ ID NO: 1574 for HPV39, SEQ ID NO: 1584 for HPV45, SEQ ID NO: 1598 for HPV51, SEQ ID NO: 1608 for HPV52, SEQ ID NO: 1624 for HPV56, SEQ ID NO: 1640 for HPV58, SEQ ID NO: 1653 for HPV59, SEQ ID NO: 1667 for HPV66, SEQ ID NO: 1682 for HPV68, SEQ ID NO: 1692 for HPV73 and SEQ ID NO: 1705 for HPV82,
the amplicons corresponding to the splice junction i=2 are respectively SEQ ID NO: 1502 for HPV16, SEQ ID NO: 1516 for HPV18, SEQ ID NO: 1534 for HPV31, SEQ ID NO: 1548 for HPV33, SEQ ID NO: 1561 for HPV35, absent for HPV39, SEQ ID NO: 1585 for HPV45, absent for HPV51, SEQ ID NO: 1609 for HPV52, SEQ ID NO: 1625 for HPV56, SEQ ID NO: 1641 for HPV58, SEQ ID NO: 1654 for HPV59, SEQ ID NO: 1668 for HPV66, absent for HPV68, absent for HPV73 and absent for HPV82,
the amplicons corresponding to the splice junction i=3 are respectively SEQ ID NO: 1503 for HPV16, SEQ ID NO: 1517 for HPV18, SEQ ID NO: 1535 for HPV31, SEQ ID NO: 1549 for HPV33, SEQ ID NO: 1562 for HPV35, absent for HPV39, absent for HPV45, SEQ ID NO: 1599 for HPV51, SEQ ID NO: 1610 for HPV52, SEQ ID NO: 1626 for HPV56, SEQ ID NO: 1642 for HPV58, absent for HPV59, SEQ ID NO: 1669 for HPV66, SEQ ID NO: 1683 for HPV68, SEQ ID NO: 1693 for HPV73 and SEQ ID NO: 1706 for HPV82,
the amplicons corresponding to the splice junction i=4 are respectively SEQ ID NO: 1504 for HPV16, SEQ ID NO: 1518 for HPV18, SEQ ID NO: 1536 for HPV31, SEQ ID NO: 1550 for HPV33, SEQ ID NO: 1563 for HPV35, SEQ ID NO: 1576 for HPV39, SEQ ID NO: 1587 for HPV45, SEQ ID NO: 1600 for HPV51, SEQ ID NO: 1611for HPV52, SEQ ID NO: 1627 for HPV56, SEQ ID NO: 1643 for HPV58, SEQ ID NO: 1656 for HPV59, SEQ ID NO: 1671 for HPV66, SEQ ID NO: 1684 for HPV68, SEQ ID NO: 1694 for HPV7 and SEQ ID NO: 1707 for HPV82,
the amplicons corresponding to the splice junction i=5 are respectively SEQ ID NO: 1505 for HPV16, SEQ ID NO: 1519 for HPV18, SEQ ID NO: 1537 for HPV31, SEQ ID NO: 1551 for HPV33, SEQ ID NO: 1564 for HPV35, absent for HPV39, SEQ ID NO: 1588 for HPV45, absent for HPV51, SEQ ID NO: 1612 for HPV52, SEQ ID NO: 1628 for HPV56, SEQ ID NO: 1644 for HPV58, SEQ ID NO: 1657 for HPV59, SEQ ID NO: 1672 for HPV66, absent for HPV68, absent for HPV73 and absent for HPV82,
the amplicons corresponding to the splice junction i=6 are respectively SEQ ID NO: 1506 for HPV16, SEQ ID NO: 1520 for HPV18, SEQ ID NO: 1538 for HPV31, SEQ ID NO: 1552 for HPV33, SEQ ID NO: 1565 for HPV35, absent for HPV39, absent for HPV45, SEQ ID NO: 1601 for HPV51, SEQ ID NO: 1613 for HPV52, SEQ ID NO: 1629 for HPV56, SEQ ID NO: 1645 for HPV58, absent for HPV59, absent for HPV66, SEQ ID NO: 1686 for HPV68, SEQ ID NO: 1695 for HPV73 and SEQ ID NO: 1708 for HPV82,
the amplicons corresponding to the splice junction i=7 are respectively SEQ ID NO: 1507 for HPV16, SEQ ID NO: 1521 for HPV18, SEQ ID NO: 1539 for HPV31, SEQ ID NO: 1553 for HPV33, SEQ ID NO: 1566 for HPV35, SEQ ID NO: 1578 for HPV39, SEQ ID NO: 1590 for HPV45, SEQ ID NO: 1602 for HPV51, SEQ ID NO: 1614 for HPV52, SEQ ID NO: 1630 for HPV56, SEQ ID NO: 1646 for HPV58, absent for HPV59, SEQ ID NO: 1674 for HPV66, SEQ ID NO: 1685 for HPV68, SEQ ID NO: 1696 for HPV73 and SEQ ID NO: 1709 for HPV82,
the amplicons corresponding to the splice junction i=8 are respectively SEQ ID NO: 1508 for HPV16, absent for HPV18, SEQ ID NO: 1540 for HPV31, SEQ ID NO: 1554 for HPV33, absent for HPV35, absent for HPV39, absent for HPV45, absent for HPV51, SEQ ID NO: 1615 for HPV52, SEQ ID NO: 1631 for HPV56, SEQ ID NO: 1647 for HPV58, absent for HPV59, SEQ ID NO: 1675 for HPV66, absent for HPV68, SEQ ID NO: 1697 for HPV73 and SEQ ID NO: 1710 for HPV82,
the amplicons corresponding to the splice junction i=9 are respectively SEQ ID NO: 1509 for HPV16, SEQ ID NO: 1522 for HPV18, SEQ ID NO: 1541 for HPV31, absent for HPV33, absent for HPV35, SEQ ID NO: 1579 for HPV39, SEQ ID NO: 1591 for HPV45, SEQ ID NO: 1603 for HPV51, SEQ ID NO: 1616 for HPV52, SEQ ID NO: 1632 for HPV56, absent HPV58, SEQ ID NO: 1659 for HPV59, SEQ ID NO: 1676 for HPV66, SEQ ID NO: 1687 for HPV68, SEQ ID NO: 1698 for HPV73 and SEQ ID NO: 1711 for HPV82
the amplicons corresponding to the splice junction i=10 are respectively absent for HPV16, SEQ ID NO: 1523 for HPV18, absent for HPV31, absent for HPV33, absent for HPV35, absent for HPV39, absent for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=11 are respectively SEQ ID NO: 1510 for HPV16, SEQ ID NO: 1524 for HPV18, SEQ ID NO: 1542 for HPV31, SEQ ID NO: 1555 for HPV33, SEQ ID NO: 1567 for HPV35, SEQ ID NO: 1580 for HPV39, SEQ ID NO: 1592 for HPV45, SEQ ID NO: 1604 for HPV51, SEQ ID NO: 1617 for HPV52, SEQ ID NO: 1633 for HPV56, SEQ ID NO: 1648 for HPV58, SEQ ID NO: 1660 for HPV59, SEQ ID NO: 1677 for HPV66, SEQ ID NO: 1688 for HPV68, SEQ ID NO: 1699 for HPV73 and SEQ ID NO: 1712 for HPV82,
the amplicons corresponding to the splice junction i=12 are respectively absent for HPV16, SEQ ID NO: 1525 for HPV18, absent for HPV31, absent for HPV33, SEQ ID NO: 1568 or HPV35, absent for HPV39, absent for HPV45, absent for HPV51, SEQ ID NO: 1618 for HPV52, SEQ ID NO: 1634 for HPV56, absent for HPV58, SEQ ID NO: 1661 for HPV59, absent for HPV66, absent for HPV68, SEQ ID NO: 1700 for HPV73, absent for HPV82,
the amplicons corresponding to the splice junction i=13 are respectively absent for HPV16, SEQ ID NO: 1526 for HPV18, absent for HPV31, absent for HPV33, absent for HPV35, absent for HPV39, SEQ ID NO: 1593 for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=14 are respectively absent for HPV16, SEQ ID NO: 1527 for HPV18, absent for HPV31, absent for HPV33, absent for HPV35, absent for HPV39, absent for HPV45, absent for HPV51, absent for HPV52, absent for HPV56, absent for HPV58, absent for HPV59, absent for HPV66, absent for HPV68, absent for HPV73 and absent for HPV82,
the amplicons corresponding to the splice junction i=15 are respectively SEQ ID NO: 1511 for HPV16, SEQ ID NO: 1528 for HPV18, SEQ ID NO: 1543 for HPV31, SEQ ID NO: 1556 for HPV33, SEQ ID NO: 1569 for HPV35, SEQ ID NO: 1581 for HPV39, SEQ ID NO: 1594 for HPV45, SEQ ID NO: 1605 for HPV51, SEQ ID NO: 1619 for HPV52, SEQ ID NO: 1635 for HPV56, SEQ ID NO: 1649 for HPV58, SEQ ID NO: 1662 for HPV59, SEQ ID NO: 1678 for HPV66, SEQ ID NO: 1689 for HPV68, SEQ ID NO: 1701 for HPV73 and SEQ ID NO: 1713 for HPV82,
the amplicons corresponding to the splice junction i=16 are respectively SEQ ID NO: 1512 for HPV16, SEQ ID NO: 1529 for HPV18, SEQ ID NO: 1544 for HPV31, SEQ ID NO: 1557 for HPV33, SEQ ID NO: 1570 for HPV35, absent for HPV39, SEQ ID NO: 1595 for HPV45, absent for HPV51, SEQ ID NO: 1620 for HPV52, SEQ ID NO: 1636 for HPV56, SEQ ID NO: 1650 for HPV58, SEQ ID NO: 1663 for HPV59, SEQ ID NO: 1679 for HPV66, absent for HPV68, absent for HPV73 and absent for HPV82,
the amplicons corresponding to the splice junction i=17 are respectively SEQ ID NO: 1513 for HPV16, absent for HPV18, SEQ ID NO: 1545 for HPV31, SEQ ID NO: 1558 for HPV33, SEQ ID NO: 1571 for HPV35, absent for HPV39, absent for HPV45, SEQ ID NO: 1606 for HPV51, SEQ ID NO: 1621 for HPV52, SEQ ID NO: 1637 for HPV56, SEQ ID NO: 1651 for HPV58, absent for HPV59, absent for HPV66, SEQ ID NO: 1690 for HPV68, SEQ ID NO: 1702 for HPV73 and SEQ ID NO: 1714 for HPV82,
the amplicons corresponding to the splice junction i=18 are respectively SEQ ID NO:1514 for HPV16, SEQ ID NO: 1531 for HPV18, SEQ ID NO: 1546 for HPV31, SEQ ID NO: 1559 for HPV33, SEQ ID NO: 1572 for HPV35, SEQ ID NO: 1583 for HPV39, SEQ ID NO: 1597 for HPV45, SEQ ID NO: 1607 for HPV51, SEQ ID NO: 1622 for HPV52, SEQ ID NO: 1638 for HPV56, SEQ ID NO: 1652 for HPV58, SEQ ID NO: 1665 for HPV59, SEQ ID NO: 1681 for HPV66, SEQ ID NO: 1691 for HPV68, SEQ ID NO: 1703 for HPV73 and SEQ ID NO: 1715 for HPV82,
the amplicons corresponding to the splice junction i=19 are respectively absent for HPV16, SEQ ID NO: 1532 for HPV18, absent for HPV31, absent for HPV33, SEQ ID NO: 1573 for HPV35, absent for HPV39, absent for HPV45, absent for HPV51, SEQ ID NO: 1623 for HPV52, SEQ ID NO: 1639 for HPV56, absent for HPV58, SEQ ID NO: 1666 for HPV59, absent for HPV66, absent for HPV68, SEQ ID NO: 1704 for HPV73 and absent for HPV82,
the amplicons corresponding to the splice junction i=20 are respectively absent for HPV16, absent for HPV18, absent for HPV31, absent for HPV33, absent for HPV35, SEQ ID NO: 1577 for HPV39, absent for HPV45, absent for HPV51, absent for HPV52, absent for HPV56, absent for HPV58, SEQ ID NO: 1658 for HPV59, absent for HPV66, absent for HPV68, absent for HPV73 and absent for HPV82,
the amplicons corresponding to the splice junction i=21 are respectively absent for HPV16, absent for HPV18, absent for HPV31, absent for HPV33, absent for HPV35, SEQ ID NO: 1582 for HPV39, absent for HPV45, absent for HPV51, absent for HPV52, absent for HPV56, absent for HPV58, SEQ ID NO: 1664 for HPV59, absent for HPV66, absent for HPV68, absent for HPV73 and absent for HPV82,
the amplicons corresponding to the splice junction i=22 are respectively absent for HPV16, absent for HPV18, absent for HPV31, absent for HPV33, absent for HPV35, SEQ ID NO: 1575 for HPV39, absent for HPV45, absent for HPV51, absent for HPV52, absent for HPV56, absent for HPV58, SEQ ID NO: 1655 for HPV59, absent for HPV66, absent for HPV68, absent for HPV73 and absent for HPV82,
the amplicons corresponding to the splice junction i=23 are respectively absent for HPV16, absent for HPV18, absent for HPV31, absent for HPV33, absent for HPV35, absent for HPV39, SEQ ID NO: 1586 for HPV45, absent for HPV51, absent for HPV52, absent for HPV56, absent for HPV58, absent for HPV59, SEQ ID NO: 1670 for HPV66, absent for HPV68, absent for HPV73 and absent for HPV82,
the amplicons corresponding to the splice junction i=24 are respectively absent for HPV16, absent for HPV18, absent for HPV31, absent for HPV33, absent for HPV35, absent for HPV39, SEQ ID NO: 1589 for HPV45, absent for HPV51, absent for HPV52, absent for HPV56, absent for HPV58, absent for HPV59, SEQ ID NO: 1673 for HPV66, absent for HPV68, absent for HPV73 and absent for HPV82,
the amplicons corresponding to the splice junction i=25 are respectively absent for HPV16, SEQ ID NO: 1530 for HPV18, absent for HPV31, absent for HPV33, absent for HPV35, absent for HPV39, SEQ ID NO: 1596 HPV45, absent for HPV51, absent for HPV52, absent for HPV56, absent for HPV58, absent for HPV59, SEQ ID NO: 1680 for HPV66, absent for HPV68, absent for HPV73 and absent for HPV82.

21. A composition of primers according to claim 16.

22. A kit comprising the composition of primers according to claim 16 and optionally reagents for cDNA amplification.

23. Use of the composition of the primers according to claim 16.

24. An in vitro method for HPV typing in a biological sample comprising the steps of:
(a) extraction of RNA from the biological sample,
(b) reverse transcription of the RNA so as to generate cDNA,
(c) amplification of the cDNA generated at step (b) with a composition of primers so as to produce amplicon(s),
(d) quantification of the expression level of each amplicon and
(e) for each HPV type, a step of:
comparing the expression level of all amplicons specific of said HPV type with a reference value, wherein if the expression level of all the amplicons specific of said HPV type is higher than the reference value, it is indicative of the presence of said HPV type in the biological sample,
wherein the composition of primers comprises a first set of pairs of primers, called splice junctions set of primers, which comprises:
at least 2 pairs of primers of a first subset of HPV16 specific pairs of primers able to produce the amplicons having the nucleic acid sequence selected from the group consisting of SEQ ID NO: 1501 to SEQ ID NO: 1514; and
at least 2 pairs of primers of a second subset of HPV18 specific pairs of primers able to produce the amplicons having the nucleic acid sequence selected from the group consisting of SEQ ID NO: 1515 to SEQ ID NO: 1532; and
at least 2 pairs of primers of a third subset of HPV31 specific pairs of primers able to produce the amplicons having the nucleic acid sequence selected from the group consisting of SEQ ID NO: 1533 to SEQ ID NO: 1546; and
at least 2 pairs of primers of a fourth subset of HPV33 specific pairs of primers able to produce the amplicons having the nucleic acid sequence selected from the group consisting of SEQ ID NO:1547 to SEQ ID NO:1559; and
at least 2 pairs of primers of a fifth subset of HPV35 specific pairs of primers able to produce the amplicons having the nucleic acid sequence selected from the group consisting of SEQ ID NO: 1560 to SEQ ID NO: 1573; and
at least 2 pairs of primers of a sixth subset of HPV39 specific pairs of primers able to produce the amplicons having the nucleic acid sequence selected from the group consisting of SEQ ID NO: 1574 to SEQ ID NO: 1583; and
at least 2 pairs of primers of a seventh subset of HPV45 specific pairs of primers able to produce the amplicons having the nucleic acid sequence selected from the group consisting of SEQ ID NO: 1584 to SEQ ID NO: 1597; and
at least 2 pairs of primers of a eighth subset HPV51 specific of pairs of primers able to produce the amplicons having the nucleic acid sequence selected from the group consisting of SEQ ID NO: 1598 to SEQ ID NO: 1607; and
at least 2 pairs of primers of a ninth subset of HPV52 specific pairs of primers able to produce the amplicons having the nucleic acid sequence selected from the group consisting of SEQ ID NO: 1608 to SEQ ID NO: 1623; and
at least 2 pairs of primers of a tenth subset HPV56 specific of pairs of primers able to produce the amplicons having the nucleic acid sequence selected from the group consisting of SEQ ID NO: 1624 to SEQ ID NO: 1639; and
at least 2 pairs of primers of an eleventh subset EIPV58 specific of pairs of primers able to produce the amplicons having the nucleic acid sequence selected from the group consisting of SEQ ID NO:1640 to SEQ ID NO:1652; and
at least 2 pairs of primers of a twelfth subset of HPV59 specific pairs of primers able to produce the amplicons having the nucleic acid sequence selected from the group consisting of SEQ ID NO: 1653 to SEQ ID NO: 1666; and
at least 2 pairs of primers of a thirteenth subset of HPV66 specific pairs of primers able to produce the amplicons having the nucleic acid sequence selected from the group consisting of SEQ ID NO: 1667 to SEQ ID NO: 1681, and optionally:
at least 2 pairs of primers of a fourteenth subset of HPV68 specific pairs of primers able to produce the amplicons having the nucleic acid sequence selected from the group consisting of SEQ ID NO: 1682 to SEQ ID NO: 1691; and/or
at least 2 pairs of primers of a fifteenth subset of HPV73 specific pairs of primers able to produce the amplicons having the nucleic acid sequence selected from the group consisting of SEQ ID NO: 1692 to SEQ ID NO: 1704; and/or
at least 2 pairs of primers of a sixteenth subset of HPV82 specific pairs of primers able to produce the amplicons having the nucleic acid sequence selected from the group consisting of SEQ ID NO: 1705 to SEQ ID NO: 1715.

25. The in vitro method for HPV typing according to claim 24, wherein the splice junctions set of primers comprises at least 10 pairs of primers of the first to the thirteenth subsets of pairs of primers and optionally at least 10 pairs of primers of the fourteenth and/or the fifteenth and/or the sixteenth subsets of pairs of primers.

26. The in vitro method for HPV typing according to claim 24, wherein the composition of primers comprises an additional set of primers selected from the group consisting of:
a second set of primers, called unsplice junctions set of primers wherein the primers target high risk and optionally of putative high risk HPV genomic regions spanning either splice donor or splice acceptor sites in the absence of any splice event,
a third set of primers, called genomic set of primers wherein the primers target high risk and optionally of putative high risk HPV genomic regions away from any splice donor or splice acceptor sites, and
a fourth set of primers, called fusion set of primers wherein the primers target high risk and optionally of putative high risk HPV fusion transcripts.
